(19) United States
(12) Patent Application Publication
(10) Pub. No.: US 2026/0157109 A1
(43) Pub. Date: Jun. 4, 2026

(54) ORGANIC COMPOUNDS, LIGHT-EMITTING ELEMENTS, AND DISPLAY PANELS

(71) Applicant: Shenzhen China Star Optoelectronics Semiconductor Display Technology Co., Ltd., Shenzhen, Guangdong (CN)

(72) Inventors: Yuanzuo ZOU, Shenzhen, Guangdong (CN); Shihao ZHOU, Shenzhen, Guangdong (CN); Ruifeng HE, Shenzhen, Guangdong (CN); Jingyao SONG, Shenzhen, Guangdong (CN)

(73) Assignee: Shenzhen China Star Optoelectronics Semiconductor Display Technology Co., Ltd., Shenzhen, Guangdong (CN)

(21) Appl. No.: 18/695,938

(22) PCT Filed: Feb. 2, 2024
(86) PCT No.: PCT/CN2024/075390

§ 371 Date: Mar. 27, 2024

(30) Foreign Application Priority Data
Jan. 18, 2024 (CN) 202410075231.2

(51) Int. Cl. H10K 85/60; C07B 59/00; C07C 211/61; C07D 209/88; C07D 307/77; C07D 307/91; C07D 333/50; C07D 333/76; H10K 50/15

(52) U.S. Cl. CPC H10K 85/633; C07B 59/001; C07C 211/61; C07D 209/88; C07D 307/77; C07D 307/91; C07D 333/50; C07D 333/76; H10K 85/636; C07C 2603/18; C07C 2603/26; C07C 2603/40; C07C 2603/86; H10K 50/15; H10K 85/622; H10K 85/623; H10K 85/626; H10K 85/6572; H10K 85/6574; H10K 85/6576

(57) ABSTRACT

Embodiments of the present disclosure discloses an organic compound, a light-emitting element, and a display panel. The organic compound has a structure as shown in formula (1):

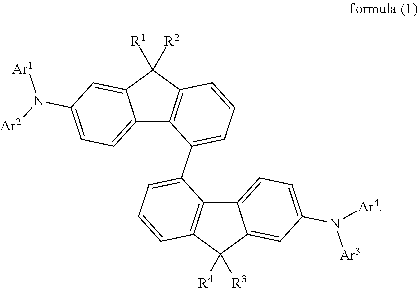

### TECHNICAL FIELD

The present disclosure relates to the field of display, and more particularly, to an organic compound, a light-emitting element, and a display panel.

### BACKGROUND

Currently, an organic electroluminescent element generally has a positive electrode, a negative electrode, and an organic layer disposed between them. Electric energy is converted into light energy by using organic substances in the organic layer, thereby realizing organic electroluminescence. In a case that voltage is applied between the positive electrode and the negative electrode of the organic electroluminescent element, holes are injected into the organic layer at the positive electrode, and electrons are injected into the organic layer at the negative electrode. The injected holes and the electrons meet to form excitons, and light is emitted when the excitons transition back to the ground state, thereby realizing light emission of the organic electroluminescent element. The organic electroluminescent element has characteristics such as self-luminescence, high brightness, high efficiency, low voltage driving, wide viewing angle, high contrast, and high response. Therefore, the organic electroluminescent element has a wide application prospect.

In order to improve the luminous efficiency of the organic electroluminescent element and prolong the service life of the organic electroluminescent element, suitable hole transport materials or the like are used in the organic functional layer of the organic electroluminescent element, so that electrons and holes are recombined in the central region of the light-emitting layer, thereby reducing exciton quenching. Existing hole transport materials still have shortcomings in carrier transport and other properties, as a result, there is still room for improvement in the luminous efficiency and the lifetime of the organic electroluminescent element.

Accordingly, there is an urgent need for an organic compound, a light-emitting element, and a display panel to solve the above-mentioned technical problems.

### Technical Problems

The present disclosure provides an organic compound, a light-emitting element, and a display panel, which can alleviate the technical problem that the luminous efficiency of the organic electroluminescent element and the service life of the organic electroluminescent element are difficult to improve due to the deficiency of existing hole transport materials in carrier transport and other properties.

### Technical Solutions

To solve the above problems, the present disclosure provides the following technical solutions:

The present disclosure provides an organic compound having a structure as shown in formula (1):

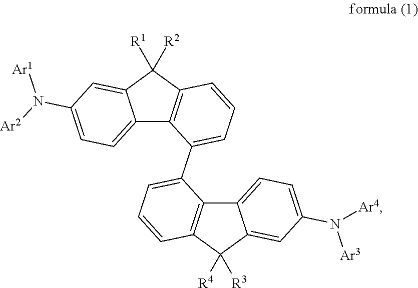

wherein Ar¹, Ar², Ar³ and Ar⁴ are each independently selected from a substituted or unsubstituted aromatic group having 6 to 20 ring atoms and a substituted or unsubstituted heteroaromatic group having 5 to 30 ring atoms; and
R¹, R², R³ and R⁴ are each independently selected from a substituted or unsubstituted methyl, a substituted or unsubstituted ethyl, a substituted or unsubstituted phenyl.

The present disclosure further provides a light-emitting element, which includes:
a pair of electrodes comprising a first electrode and a second electrode;
an organic functional layer disposed between the first electrode and the second electrode;
wherein a material of the organic functional layer comprises an organic compound having a structure as shown in formula (1):

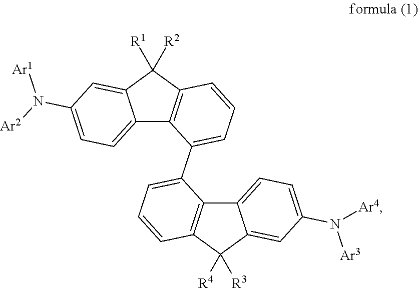

wherein Ar¹, Ar², Ar³ and Ar⁴ are each independently selected from a substituted or unsubstituted aromatic group having 6 to 20 ring atoms and a substituted or unsubstituted heteroaromatic group having 5 to 30 ring atoms; and
R¹, R², R³ and R⁴ are each independently selected from a substituted or unsubstituted methyl, a substituted or unsubstituted ethyl, a substituted or unsubstituted phenyl.

The present disclosure further provides a display panel. The display panel includes a light-emitting element including:
a pair of electrodes comprising a first electrode and a second electrode;
an organic functional layer disposed between the first electrode and the second electrode;
wherein a material of the organic functional layer comprises an organic compound having a structure as shown in formula (1):

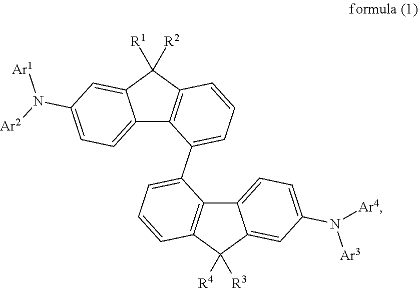

wherein Ar¹, Ar², Ar³ and Ar⁴ are each independently selected from a substituted or unsubstituted aromatic group having 6 to 20 ring atoms and a substituted or unsubstituted heteroaromatic group having 5 to 30 ring atoms; and
R¹, R², R³ and R⁴ are each independently selected from a substituted or unsubstituted methyl, a substituted or unsubstituted ethyl, a substituted or unsubstituted phenyl.

### BRIEF DESCRIPTION OF THE DRAWINGS

FIG. 1 is a schematic structural diagram of a light-emitting element according to embodiments of the present disclosure.

FIG. 2 is an optimized molecular structure and HOMO energy level distribution diagram of an organic compound M95 according to embodiments of the present disclosure.

### EMBODIMENTS OF THE INVENTION

The present disclosure provides an organic compound, a light-emitting element, and a display panel. In order to make the purpose, technical solutions, and effect of the present disclosure clear and definite, the present disclosure will be described in further detail below with reference to the accompanying drawings and embodiments. It is to be understood that the specific embodiments described herein are merely illustrative of the present disclosure and are not intended to limit the present disclosure.

In the present disclosure, aromatic groups, aromatic, aromatic ring systems have the same meaning and are interchangeable.

In the present disclosure, heteroaromatic groups, heteroaromatic, heteroaromatic ring systems have the same meaning and are interchangeable.

In the present disclosure, “substituted” means that the hydrogen atom/hydrogen atoms in a substituent is/are substituted by one or more substituents.

In the present disclosure, when the same substituent occurs multiple times, it may be independently selected from different groups, for example, when the formula contains a plurality of R, then R may be independently selected from different groups.

In the present disclosure, “substituted or unsubstituted” refers to that the defined group may be substituted or may not be substituted. When the defined group is substituted, it should be understood that it is substituted by one or more substituents R, and R is selected but not limited to, deuterium atom, cyano group, isocyano group, nitro group or halogen, alkyl groups having 1 to 20 carbon atoms, heterocyclyl groups having 3 to 20 ring atoms, aromatic groups having 6 to 20 ring atoms, heteroaryl groups having 5 to 20 ring atoms, —NR′R″, silyl group, carbonyl group, alkoxycarbonyl group, aryloxycarbonyl group, carbamoyl group, haloformyl group, formyl group, isocyanate group, thiocyanate group, isothiocyanate group, hydroxyl group, trifluoromethyl group, and the above groups may be further substituted by substituents acceptable in the art. It can be understood that R′ and R″ in —NR′R″ are each independently selected from, but not limited to, H, deuterium atom, cyano group, isocyano group, nitro group or halogen, alkyl groups having 1 to 10 carbon atoms, heterocyclyl groups having 3 to 20 ring atoms, aromatic groups having 6 to 20 ring atoms, heteroaryl groups having 5 to 20 ring atoms. Preferably, R is selected from, but not limited to, deuterium atom, cyano group, isocyano group, nitro group or halogen, alkyl groups having 1 to 10 carbon atoms, heterocyclyl groups having 3 to 10 ring atoms, aromatic groups having 6 to 20 ring atoms, heteroaryl groups having 5 to 20 ring atoms, silyl group, carbonyl group, alkoxycarbonyl group, aryloxycarbonyl group, carbamoyl group, haloformyl group, formyl group, isocyanate group, thiocyanate group, isothiocyanate group, hydroxyl group, trifluoromethyl group, and the above groups may be further substituted by substituents acceptable in the art.

In the present disclosure, “the number of ring atoms” refers to the number of atoms constituting the ring itself in a structural compound (e.g., a monocyclic compound, a fused ring compound, a cross-linked compound, a carbocyclic compound, a heterocyclic compound) in which atoms are bonded to form a ring. In a case that the ring is replaced by a substituent, the atoms contained in the substituent are not included in the ring-forming atoms. The “number of ring atoms” mentioned below has the same meaning unless otherwise specified, for example, the number of ring atoms of benzene ring is 6, the number of ring atoms of naphthalene ring is 10, and the number of ring atoms of thienyl group is 5.

In the present disclosure, “aryl groups or aromatic group” refers to an aromatic hydrocarbon group derived from an aromatic ring compound by removing one hydrogen atom. It may be a monocyclic aryl group, a fused ring aryl group or a polycyclic aryl group, and for polycyclic rings, at least one of them is an aromatic ring system. For example, “substituted or unsubstituted aryl groups having 6 to 40 ring atoms” refers to aryl groups having 6 to 40 ring atoms, preferably substituted or unsubstituted aryl groups having 6 to 30 ring atoms, more preferably substituted or unsubstituted aryl groups having 6 to 18 ring atoms, particularly preferably substituted or unsubstituted aryl groups having 6 to 14 ring atoms, and optionally the above aryl groups may be further substituted. Suitable examples include, but are not limited to, phenyl, biphenyl, terphenyl, naphthyl, anthracenyl, phenanthrenyl, fluoranthenyl, triphenylene, pyrenyl, perylene, tetraphenyl, fluorenyl, perylene, acenaphthylene, and derivatives thereof. It can be understood that a plurality of aryl groups may also be interrupted by short non-aromatic units (for example, <10% of non-H atoms, such as C, N or O atoms), such as acenaphthylene, fluorene, or 9,9-diarylfluorene, triarylamine, diaryl ether systems should also be included in the definition of aryl groups.

In the present disclosure, “heteroaryl group or heteroaromatic group” means that at least one carbon atom is replaced by a non-carbon atom on the basis of an aryl group. The non-carbon atom can be an N atom, an O atom, an S atom, or the like. For example, “substituted or unsubstituted heteroaryl groups having 5 to 40 ring atoms” refers to heteroaryl groups having 5 to 40 ring atoms, preferably substituted or unsubstituted heteroaryl groups having 6 to 30 ring atoms, more preferably substituted or unsubstituted heteroaryl groups having 6 to 18 ring atoms, particularly preferably substituted or unsubstituted heteroaryl groups having 6 to 14 ring atoms, and optionally the heteroaryl groups can be further substituted. Ssuitable examples include, but are not limited to: thienyl, furanyl, pyrrolyl, imidazolyl, diazolyl, triazolyl, imidazolyl, pyridyl, bipyridyl, pyrimidinyl, triazinyl, acridinyl, pyridazinyl, pyrazinyl, quinolinyl, isoquinolinyl, quinazolinyl, quinoxalinyl, phthalazinyl, pyridopyrimidinyl, pyridopyrazinyl, benzothienyl, benzofuranyl, indolyl, pyrroloimidazolyl, pyrrolopyrrolyl, thienopyrrolyl, thienothienyl, furopyrrolyl, furofuranyl, thienofuranyl, benzisoxazolyl, benzisothiazolyl, benzimidazolyl, o-naphthyridinyl, phenanthrenyl, primary pyridyl, quinazolinonyl, dibenzothienyl, dibenzofuranyl, carbazolyl and derivatives thereof.

In the present disclosure, “alkyl group” may represent a linear, branched and/or cyclic alkyl group. The number of carbons in an alkyl group can be 1 to 50, 1 to 30, 1 to 20, 1 to 10, or 1 to 6. A phrase containing the term, for example, “C₁₋₉ alkyl group” refers to an alkyl group containing 1 to 9 carbon atoms, which may independently be a C₁ alkyl group, a C₂ alkyl group, a C₃ alkyl group, a C₄ alkyl group, a C₅ alkyl group, a C₆ alkyl group, a C₇ alkyl group, a Cs alkyl group, or a C₉ alkyl group. Non-limiting examples of alkyl groups include methyl, ethyl, n-propyl, isopropyl, n-butyl, sec-butyl, tert-butyl, isobutyl, 2-ethylbutyl, 3,3-dimethylbutyl, n-pentyl, isopentyl, neopentyl, tert-pentyl, cyclopentyl, 1-methylpentyl, 3-methylpentyl, 2-ethylpentyl, 4-methyl-2-pentyl, n-hexyl, 1-methylhexyl, 2-ethylhexyl, 2-butylhexyl, cyclohexyl, 4-methylcyclohexyl, 4-tert-butylcyclohexyl, n-heptyl, 1-methylheptyl, 2,2-dimethylheptyl, 2-ethylheptyl, 2-butylheptyl, n-octyl, tert-octyl, 2-ethyloctyl, 2-butyloctyl, 2-hexyloctyl, 3,7-dimethyloctyl, cyclooctyl, n-nonyl, n-decyl, adamantyl, 2-ethyldecyl, 2-butyldecyl, 2-hexyldecyl, 2-octyldecyl, n-undecyl, n-dodecyl, 2-ethyldodecyl, 2-butyldodecyl, 2-hexyldodecyl, 2-octyldodecyl, n-tridecyl, n-tetradecyl, n-pentadecyl, n-hexadecyl, 2-ethylhexadecyl, 2-butylhexadecyl, 2-hexylhexadecyl, 2-octylhexadecyl, n-heptadecyl, n-octadecyl, n-nonadecyl, n-eicosyl, 2-ethyleicosyl, 2-butyleicosyl, 2-octyleicosyl, n-heneicosyl, n-docosyl, n-tricosyl, n-tetracosyl, n-pentacosyl, n-hexacosyl, n-heptacosyl, n-octacosyl, n-hexacosyl, n-triacontane, etc.

In the present disclosure, the abbreviations of the substituents shall correspond to n-normal; sec-secondary, i-iso, t-tertiary, o-ortho, m-methyl, p-phenol, Me-methyl, Et-etethyl, Pr-propyl, Bu-butyl, Am-n-pentyl, Hx-hexyl, Cy-cyclohexyl.

In the present disclosure, “amino” refers to a derivative of an amine having the structural characteristics of formula —N(X)₂, wherein each “X” is independently selected from H, substituted or unsubstituted alkyl, substituted or unsubstituted cycloalkyl, substituted or unsubstituted heterocyclyl, and the like. Non-limiting types of amine groups include —NH₂, —N(alkyl)₂, —NH(alkyl), —N(cycloalkyl)₂, —NH(cycloalkyl), —N(heterocyclyl)₂, —NH(heterocyclyl), —N(aryl)₂, —NH(aryl), —N(alkyl)(aryl), —N(alkyl)(heterocyclyl), —N(cycloalkyl)(heterocyclyl), —N(aryl)(heteroaryl), —N(alkyl)(heteroaryl), or the like.

In the present disclosure, unless otherwise defined, hydroxyl refers to —OH, carboxyl refers to —COOH, carbonyl refers to —C(═O)—, amino refers to —NH₂, formyl refers to —C(═O)H, haloformyl refers to —C(═O)Z (where Z represents halogen), carbamoyl refers to —C(═O)NH₂, isocyanate refers to —NCO, and isothiocyanate refers to —NCS.

In the present disclosure, the term “alkoxy” refers to a group having the structure “—O-alkyl”, i.e., an alkyl group as defined above is attached to other groups via an oxygen atom. Suitable examples of phrases including the term include, but are not limited to, methoxy (—O—CH₃ or —OMe), ethoxy (—O—CH₂CH₃ or -OEt), and tert-butoxy (—O—C(CH₃)₃ or -OtBu).

In the present disclosure, “*” indicates a linkage site or a fusion site.

In the present disclosure, in a case that the linkage site is not specified in a group, it means that any linkage site in a group can be used as a linkage site.

In the present disclosure, in a case that the fusion site is not specified in a group, it means that any fusion site in a group can be used as a fusion site, preferably two or more sites in the adjacent position of the group are fusion sites.

In the present disclosure, when the same group contains multiple substituents with identical symbol, each substituent may be the same or different from each other, for example, for

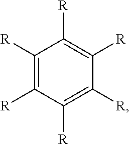

six R on the phenyl ring may be the same or different from each other.

In the present disclosure, a single bond connected to a substituent extends through the corresponding ring, meaning that the substituent may be connected to any position of the ring, for example, R in

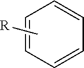

may be connected to any substitutable position of the phenyl ring. For example,

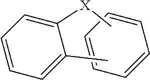

denotes that

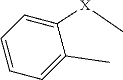

may be formed a ring with an optional substitutable position on the benzene ring

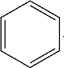

The cyclic alkyl or cycloalkyl groups according to the present disclosure have the same meaning and are interchangeable.

In the present disclosure, “adjacent groups” means that there are no substitutable sites between two substituents.

In the present disclosure, “two adjacent R₁ or R₃ or R₅ form a ring with each other” means that a ring system formed by connecting two adjacent R₁ or R₃ or R₅ to each other, and the ring system may be selected from an aliphatic hydrocarbon ring, an aliphatic heterocyclic ring, an aromatic hydrocarbon ring or an aromatic heterocyclic ring. Preferably,

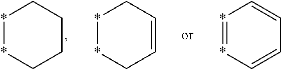

or may be formed.

Currently, due to the deficiencies of existing hole transport materials in performance such as carrier transport and charge balance adjustment, there is still room for improvement in the luminous efficiency and the lifetime of the organic electroluminescent element.

Embodiments of the present disclosure provides an organic compound, which has a structure as shown in formula (1):

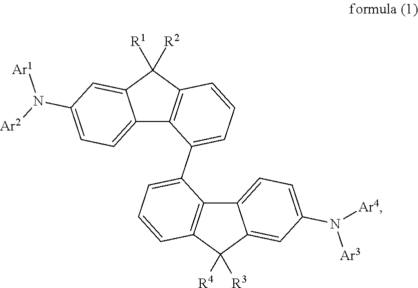

wherein Ar¹, Ar², Ar³ and Ar⁴ are each independently selected from a substituted or unsubstituted aromatic group having 6 to 20 ring atoms and a substituted or unsubstituted heteroaromatic group having 5 to 30 ring atoms; and
R¹, R², R³ and R⁴ are each independently selected from a substituted or unsubstituted methyl, a substituted or unsubstituted ethyl, a substituted or unsubstituted phenyl.

According to the present disclosure, an organic compound having a structure represented by formula (1) is used, in the structure of the organic compound, two fluorenyl groups are connected by a single bond to form an approximately orthogonal highly twisted conformation, and two fluorenyl groups are respectively connected to one aromatic amine group, so that the molecular structure is further twisted, the energy level distribution of the molecule is more uniform, and the hole transport capacity of the organic compound is stronger and the stability is higher, and the transport balance of the holes and the electrons in the light-emitting element are adjusted to be stronger, thereby improving the luminous efficiency of the light-emitting element and prolonging the service life of the light-emitting element.

In some embodiments, Ar¹, Ar², Ar³ and Ar⁴ are each independently selected from a substituted or unsubstituted aromatic group having 6 to 16 ring atoms, a substituted or unsubstituted heteroaromatic group having 5 to 20 ring atoms.

In some embodiments, Ar¹, Ar², Ar³ and Ar⁴ are each independently selected from a substituted or unsubstituted aromatic group having 6 to 16 ring atoms, a substituted or unsubstituted heteroaromatic group having 5 to 16 ring atoms.

In some embodiments, Ar¹, Ar², Ar³ and Ar⁴ are each independently selected from groups as follows:

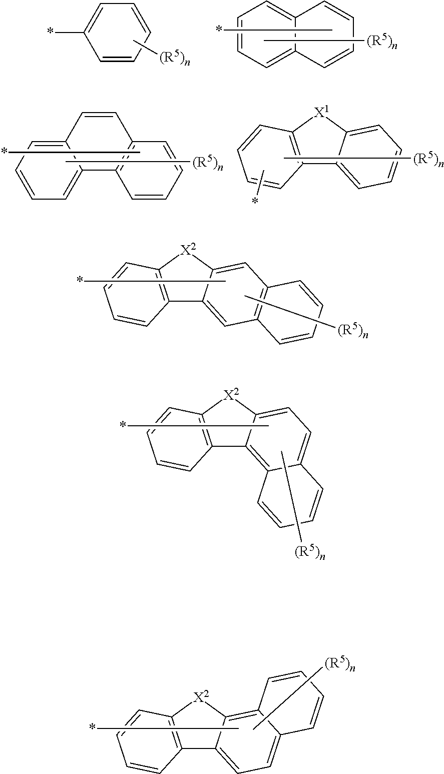

wherein X¹ and X² are each independently selected from O, S, N-Ph, CR⁶R⁷;
R⁵, R⁶ and R⁷ are each independently selected from hydrogen, deuterium, a substituted or unsubstituted linear alkyl group having 1 to 20 carbon atoms, a substituted or unsubstituted branched alkyl group or cyclic alkyl group having 3 to 20 carbon atoms, a substituted or unsubstituted aromatic group having 6 to 10 ring atoms;
N is selected from any integer from 0 to 9; and
* indicates a linkage site.

In some embodiments, X¹ is selected from O, S, N-Ph, CR⁶R⁷, and X² is selected from O, S, CR⁶R⁷.

In some embodiments, R⁵, R⁶ and R⁷ are each independently selected from hydrogen, deuterium, a substituted or unsubstituted linear alkyl having 1 to 10 carbon atoms, a substituted or unsubstituted branched alkyl or cyclic alkyl having 3 to 10 carbon atoms, a substituted or unsubstituted aromatic group having 6 to 10 ring atoms.

In some embodiments, R⁵, R⁶ and R⁷ are each independently selected from hydrogen, deuterium, a substituted or unsubstituted linear alkyl having 1 to 5 carbon atoms, a substituted or unsubstituted branched alkyl having 3 to 5 carbon atoms, a substituted or unsubstituted cyclic alkyl having 6 to 10 carbon atoms, a substituted or unsubstituted aromatic group having 6 to 10 ring atoms.

In some embodiments, each occurrence of R⁵ is independently selected from hydrogen, deuterium, a substituted or unsubstituted methyl, a substituted or unsubstituted branched alkyl or cyclic alkyl having 3 to 10 carbon atoms, a substituted or unsubstituted phenyl, a substituted or unsubstituted naphthyl.

In some embodiments, each occurrence of R⁵ is independently selected from hydrogen, deuterium, a substituted or unsubstituted methyl, a substituted or unsubstituted isopropyl, a substituted or unsubstituted tert-butyl, a substituted or unsubstituted cyclohexyl, a substituted or unsubstituted adamantyl, a substituted or unsubstituted phenyl, a substituted or unsubstituted naphthyl.

In some embodiments, each occurrence of R⁵ is independently selected from hydrogen, deuterium, an unsubstituted methyl, an unsubstituted isopropyl, an unsubstituted tert-butyl, an unsubstituted cyclohexyl, an unsubstituted adamantyl, an unsubstituted phenyl, an unsubstituted naphthyl.

In some embodiments, R⁶ and R⁷ are each independently selected from a substituted or unsubstituted methyl, a substituted or unsubstituted ethyl, a substituted or unsubstituted phenyl.

In some embodiments, R⁶ and R⁷ are each independently selected from an unsubstituted methyl, an unsubstituted ethyl, an unsubstituted phenyl.

In some embodiments, the substituted methyl, substituted ethyl, substituted isopropyl, substituted tert-butyl, substituted cyclohexyl and substituted adamantyl satisfy the following conditions: at least one hydrogen atom in the group is replaced by a deuterium atom.

In some embodiments, the substituted phenyl and substituted naphthyl satisfies the following conditions:
at least one hydrogen atom in the group is replaced by a deuterium atom; and/or,
at least one hydrogen atom in the group is replaced by a methyl.

In some embodiments, in a case that any one of R¹, R², R³ and R⁴ is selected from a substituted methyl, any one of R¹, R², R³ and R⁴ can be selected from

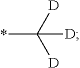

and in a case that any one of R¹, R², R³ and R⁴ is selected from a substituted phenyl, any one of R¹, R², R³ and R⁴ may be selected from:

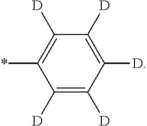

In some embodiments, n is selected from any one of 0 to 9 and n may be selected from any one of 0, 1, 2, 3, 4, 5, 6, 7, 8, 9.

In some embodiments, Ar¹, Ar², Ar³ and Ar⁴ are each independently selected from groups as follows:

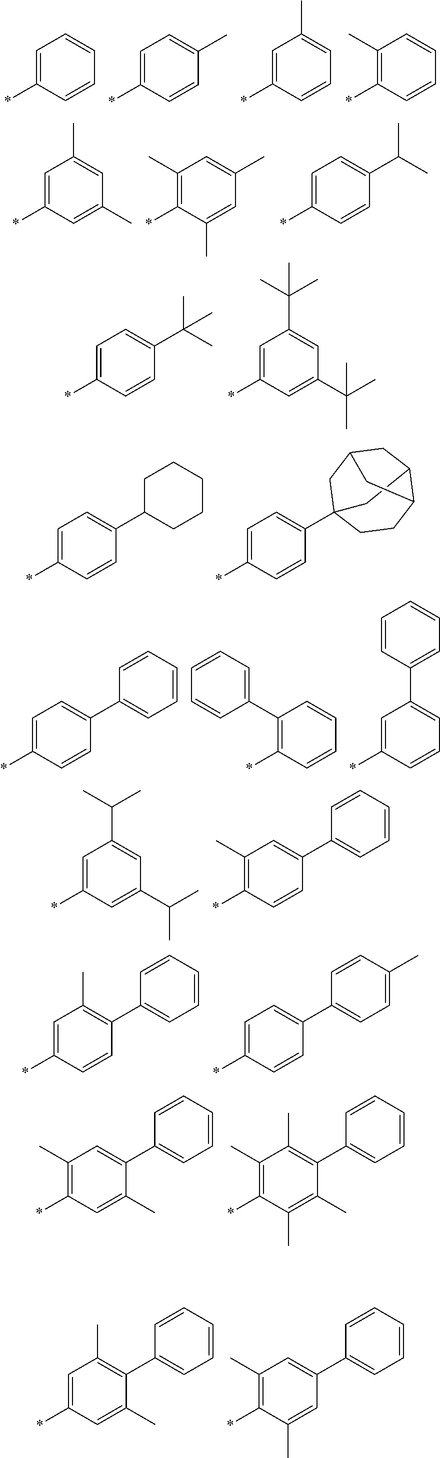

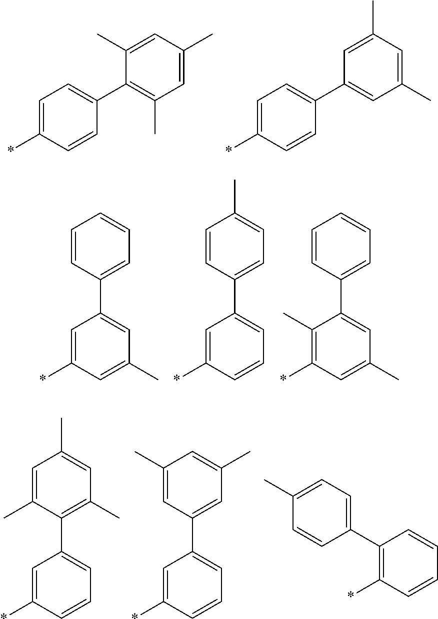

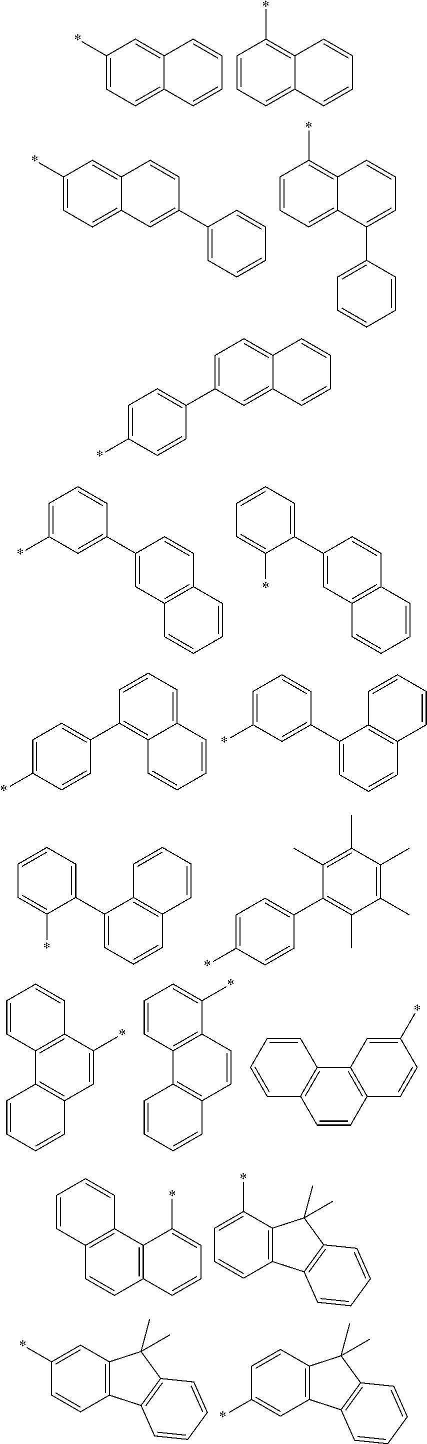

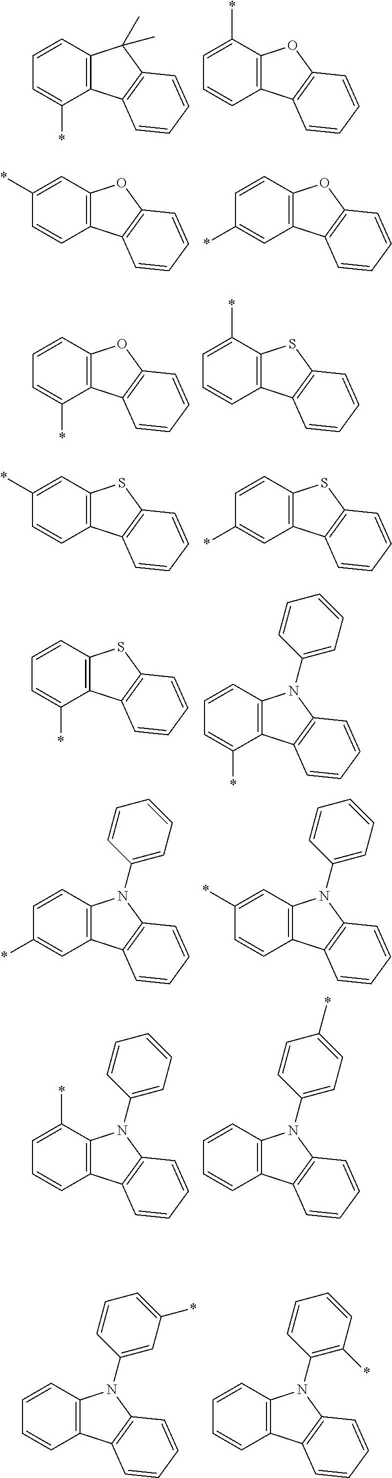

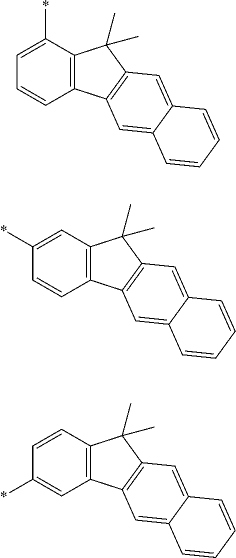 [SMILES: *c1cccc2c1C(C)(C)c1cc3ccccc3cc1-2 ; *c1ccc2c(c1)C(C)(C)c1cc3ccccc3cc1-2 ; *c1ccc2c(c1)-c1cc3ccccc3cc1C2(C)C]

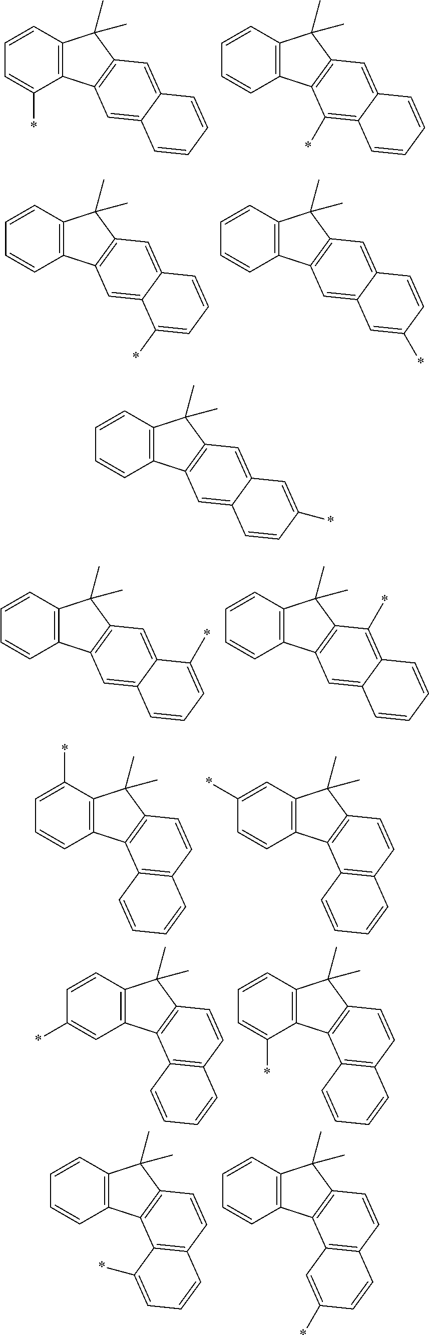

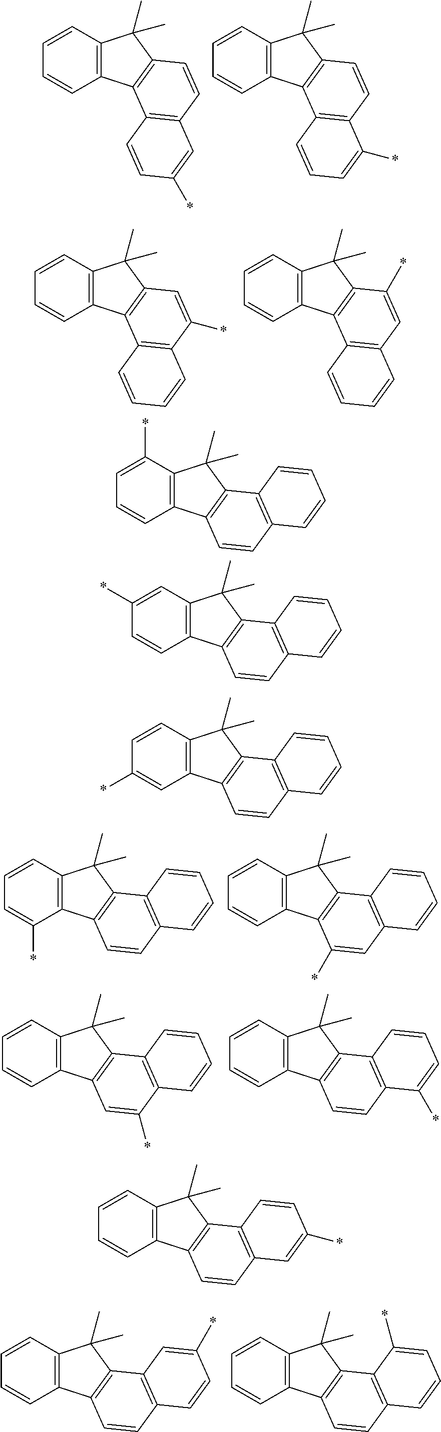

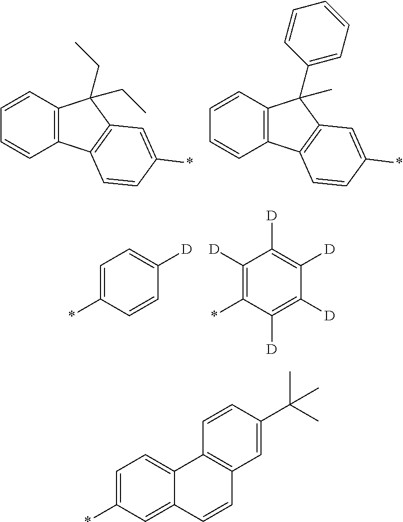

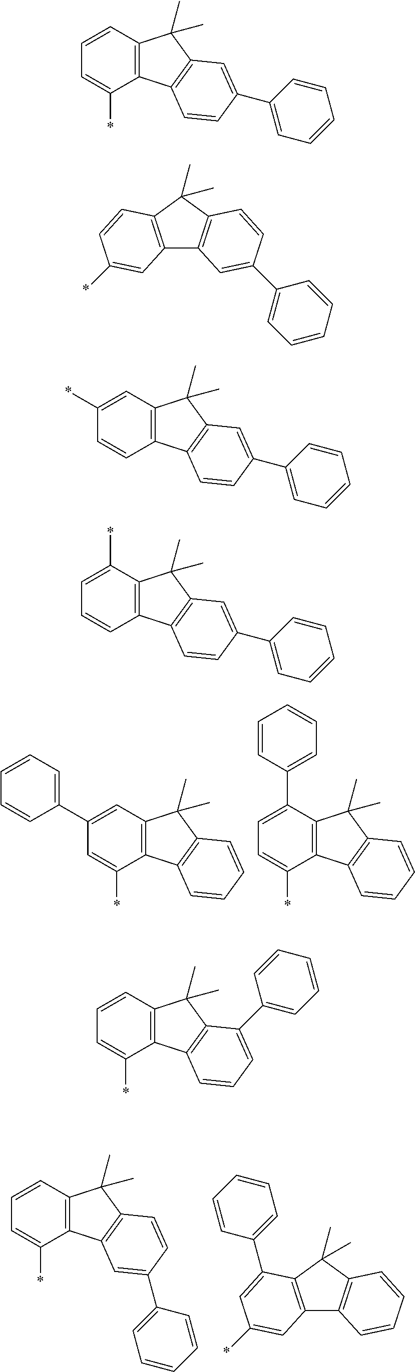

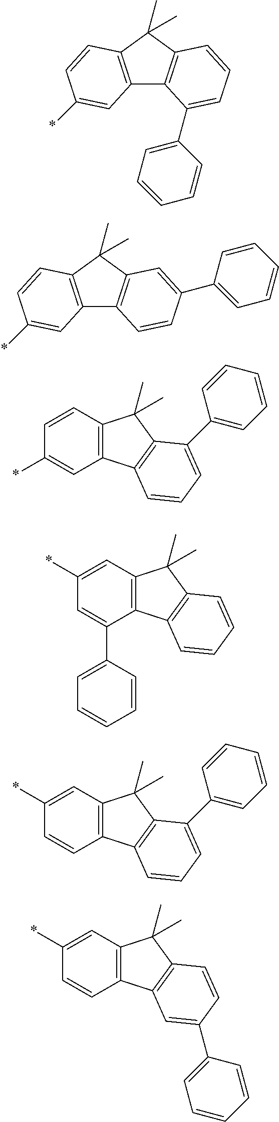

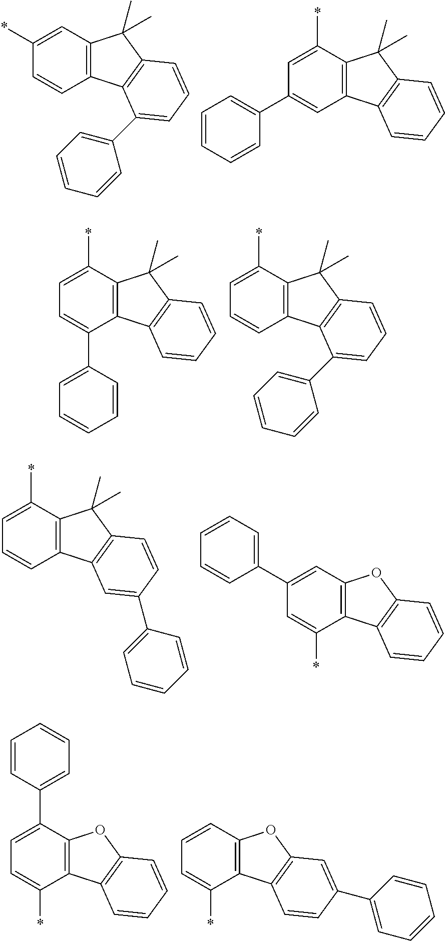

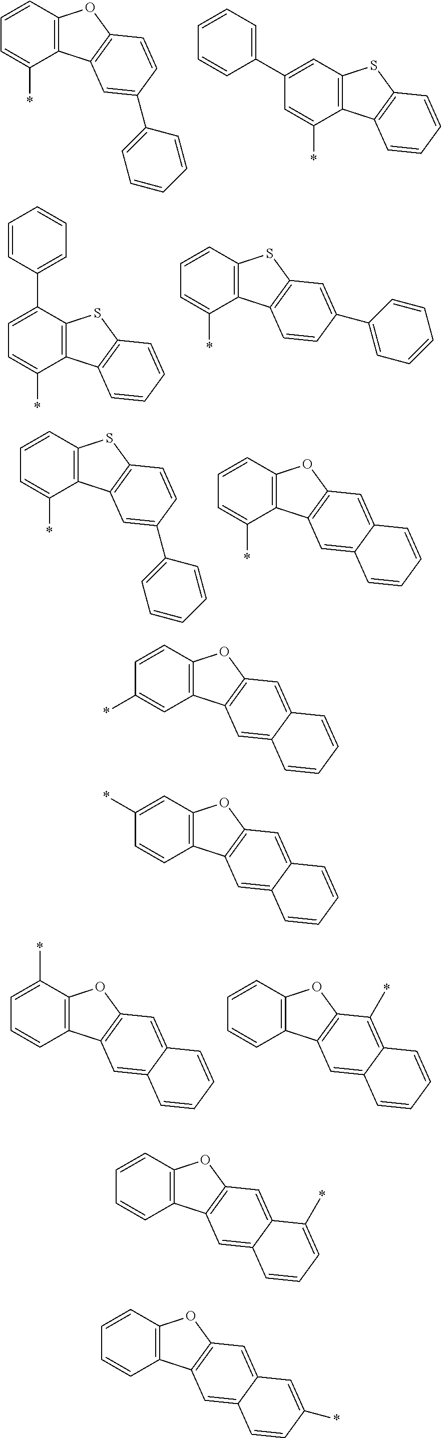

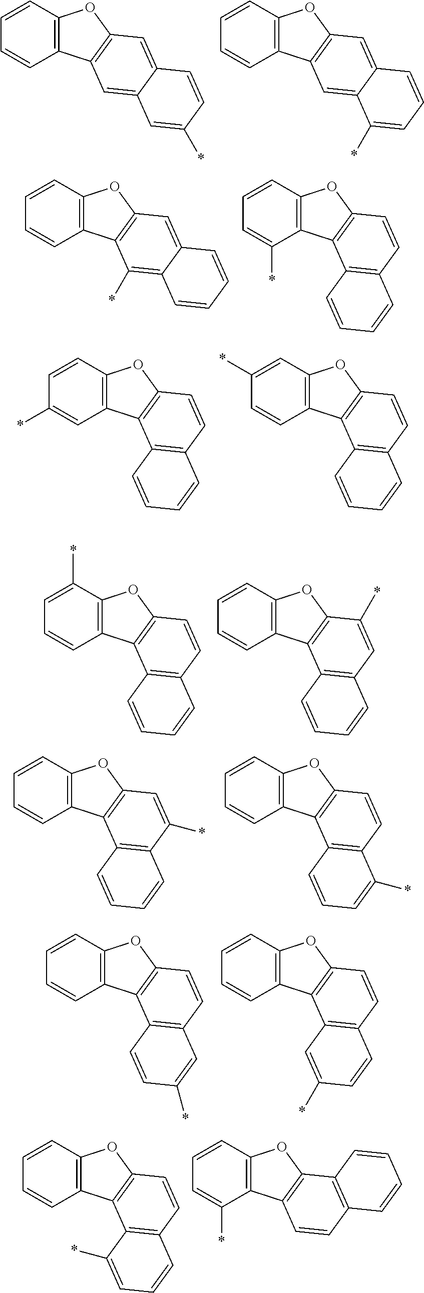

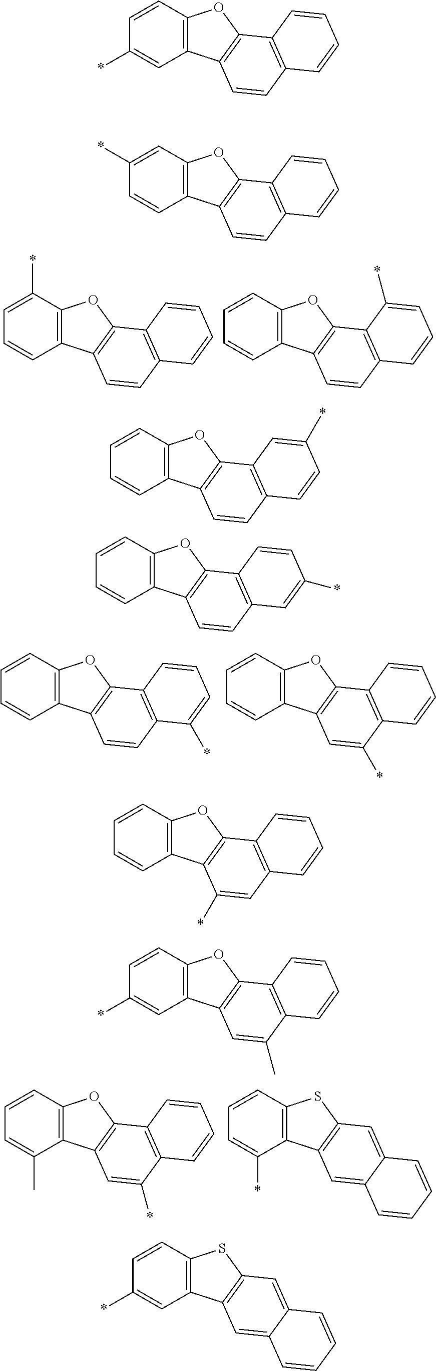

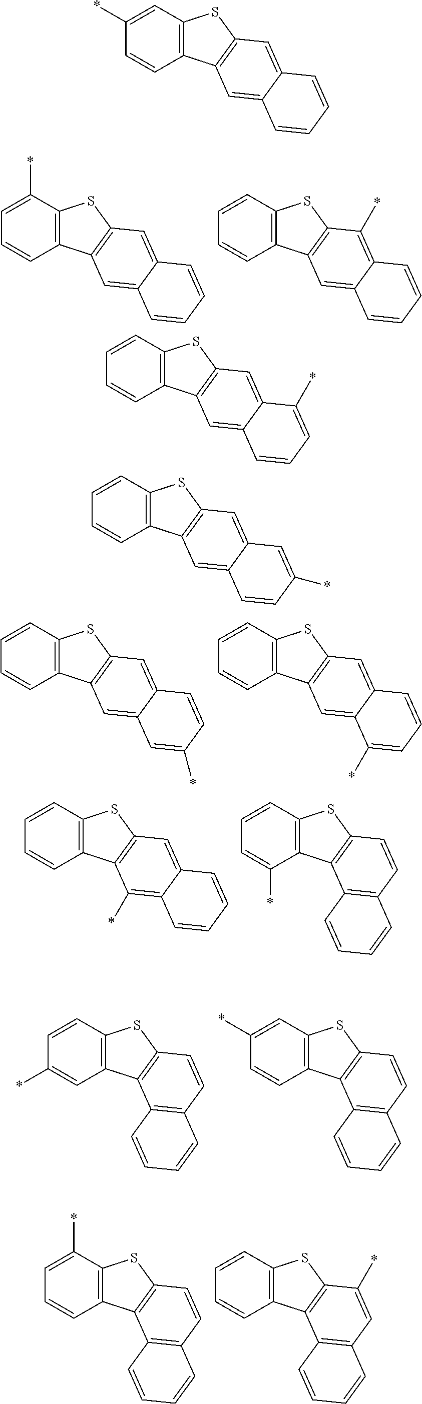

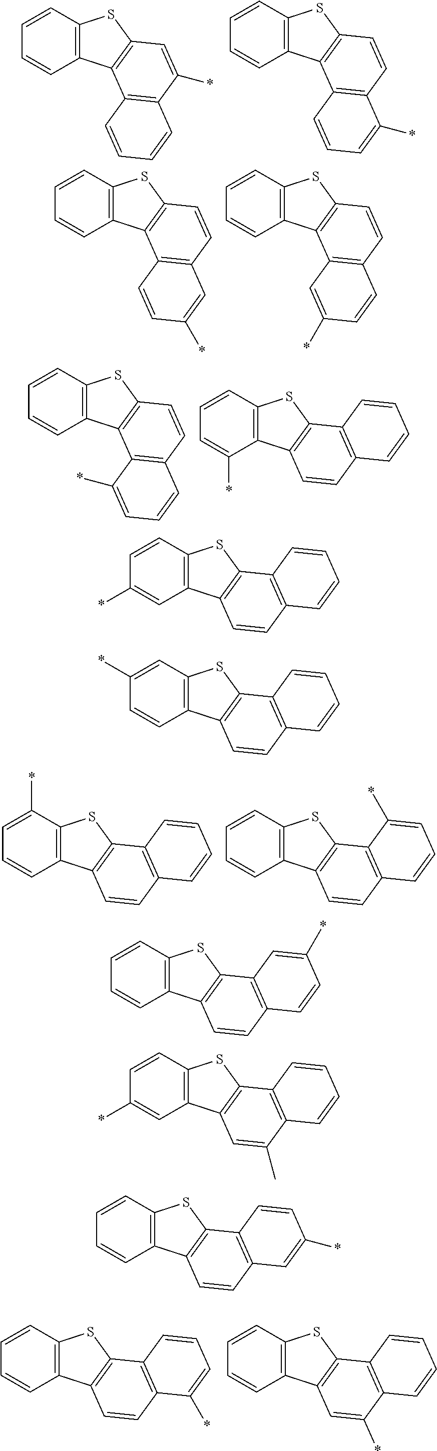

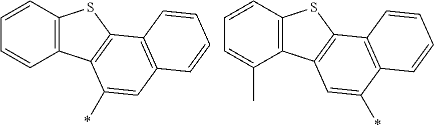

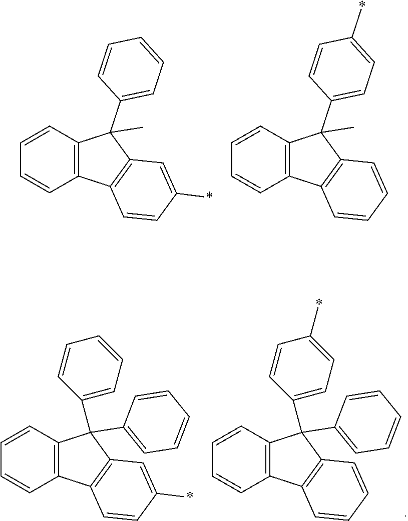

In some embodiments, Ar¹ is centrosymmetric with Ar³, and/or Ar² is centrosymmetric with Ar⁴. In a case that Ar¹ is centrosymmetric with Ar³, Ar¹ and Ar³ have the same group, and in a case that Ar² is centrosymmetric with Ar⁴, Ar² and Ar⁴ have the same group, which is beneficial to simplify the synthesis of the organic compound.

In some embodiments, the organic compound has a centrosymmetric structure, or alternatively, the organic compound has a non-centrosymmetric structure. In a case that the organic compound has a centrosymmetric structure, the structures can completely overlap before and after the organic compound molecules rotate around a point by 180 degrees. The central symmetric structure of the organic compound facilitates simplification of the synthesis of the organic compound.

In some embodiments, the organic compound is selected from the following compounds:

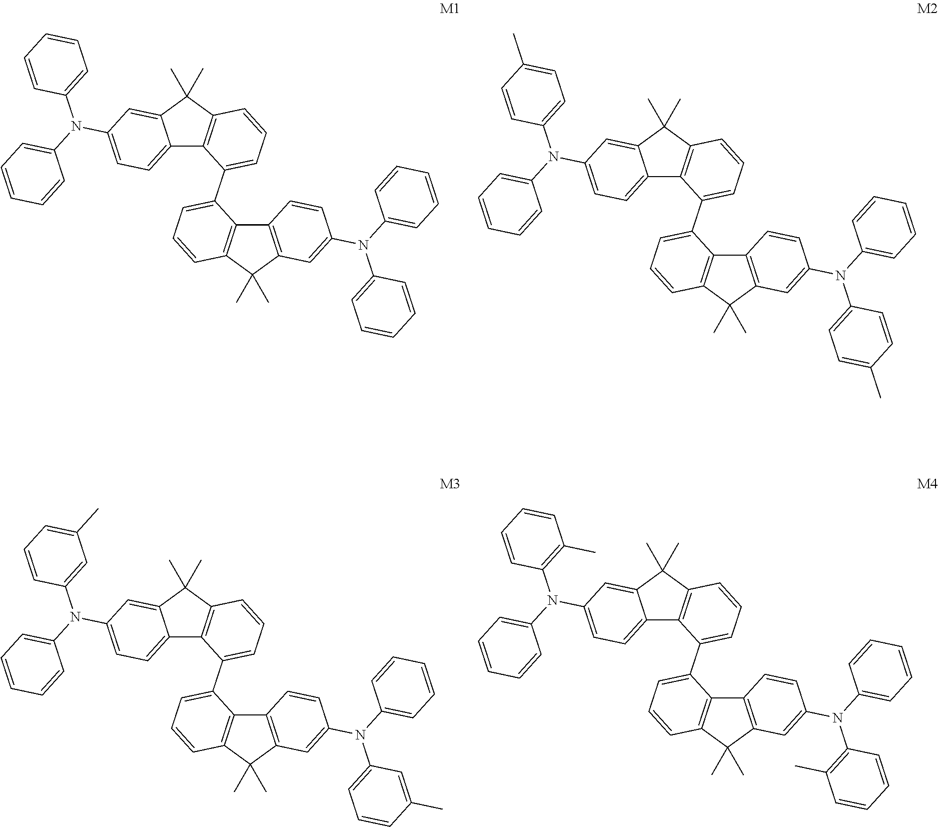

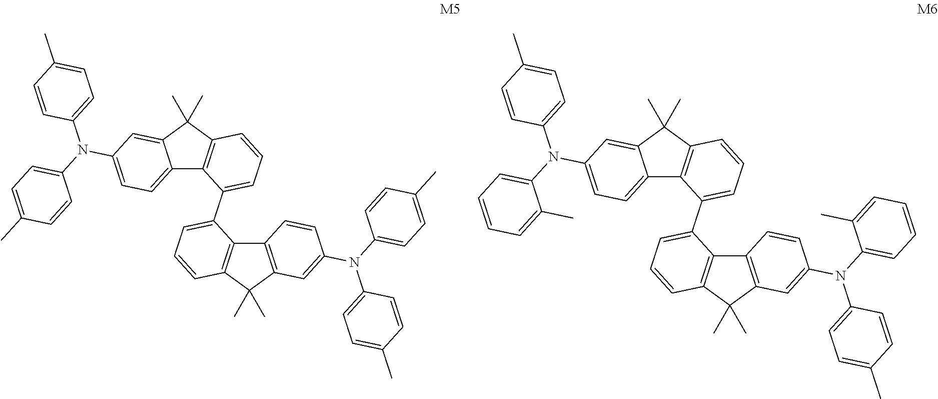

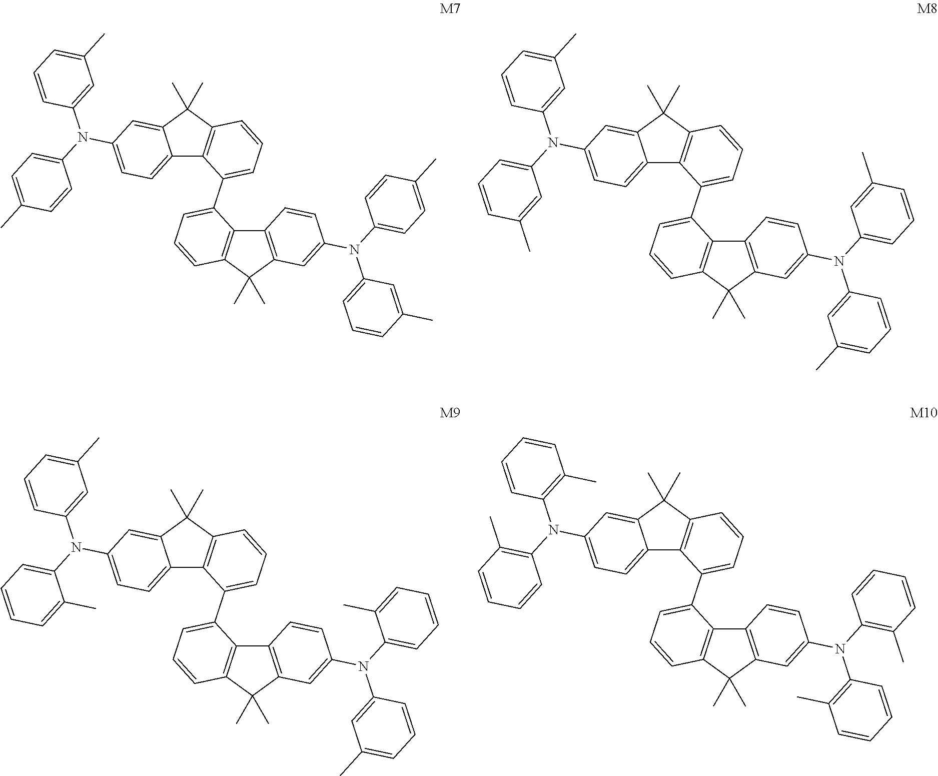

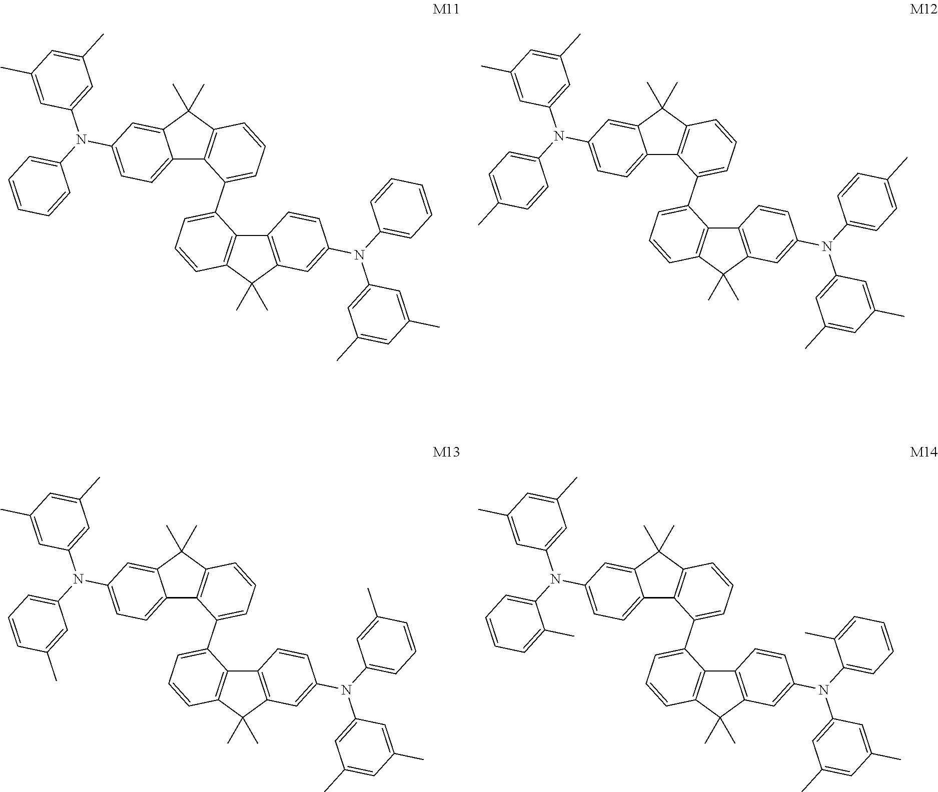

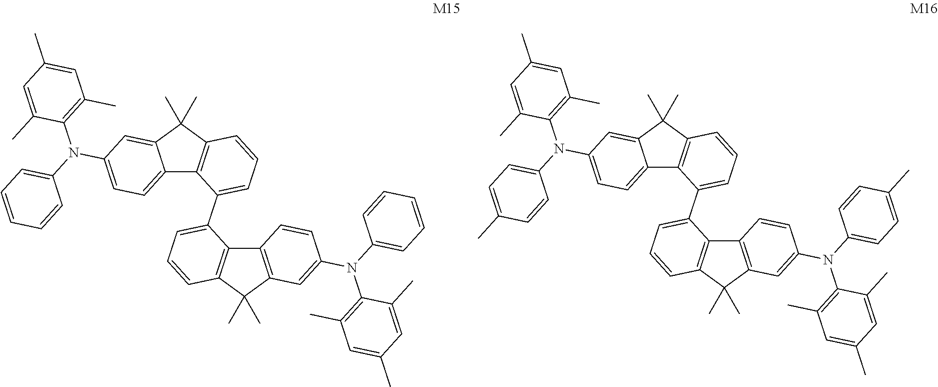

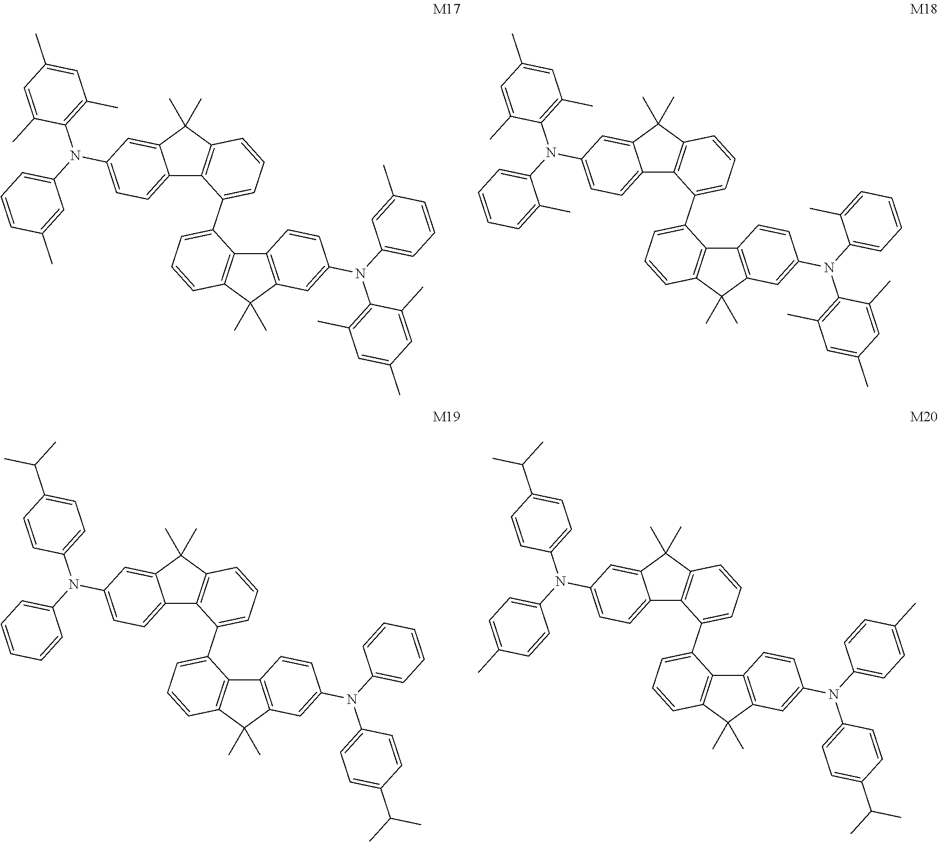

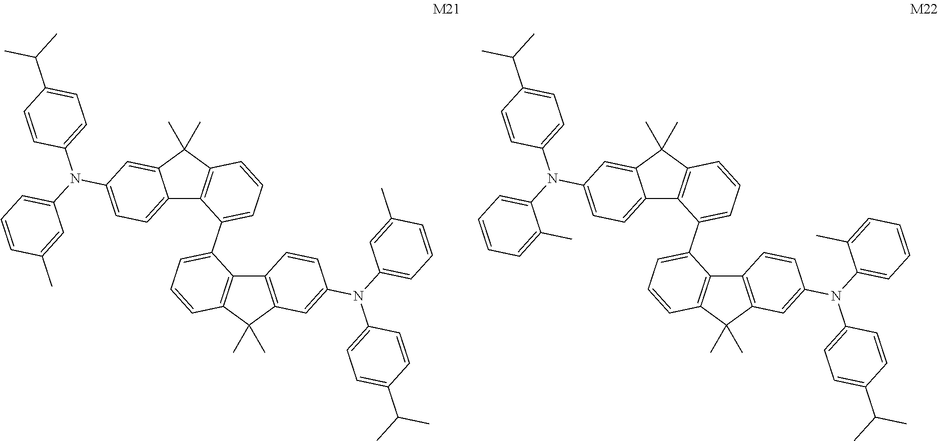 [SMILES: Cc1cccc(N(c2ccc(C(C)C)cc2)c2ccc3c(c2)C(C)(C)c2cccc(-c4cccc5c4-c4ccc(N(c6ccc(C(C)C)cc6)c6cccc(C)c6)cc4C5(C)C)c2-3)c1 ; Cc1ccccc1N(c1ccc(C(C)C)cc1)c1ccc2c(c1)C(C)(C)c1cccc(-c3cccc4c3-c3ccc(N(c5ccc(C(C)C)cc5)c5ccccc5C)cc3C4(C)C)c1-2]

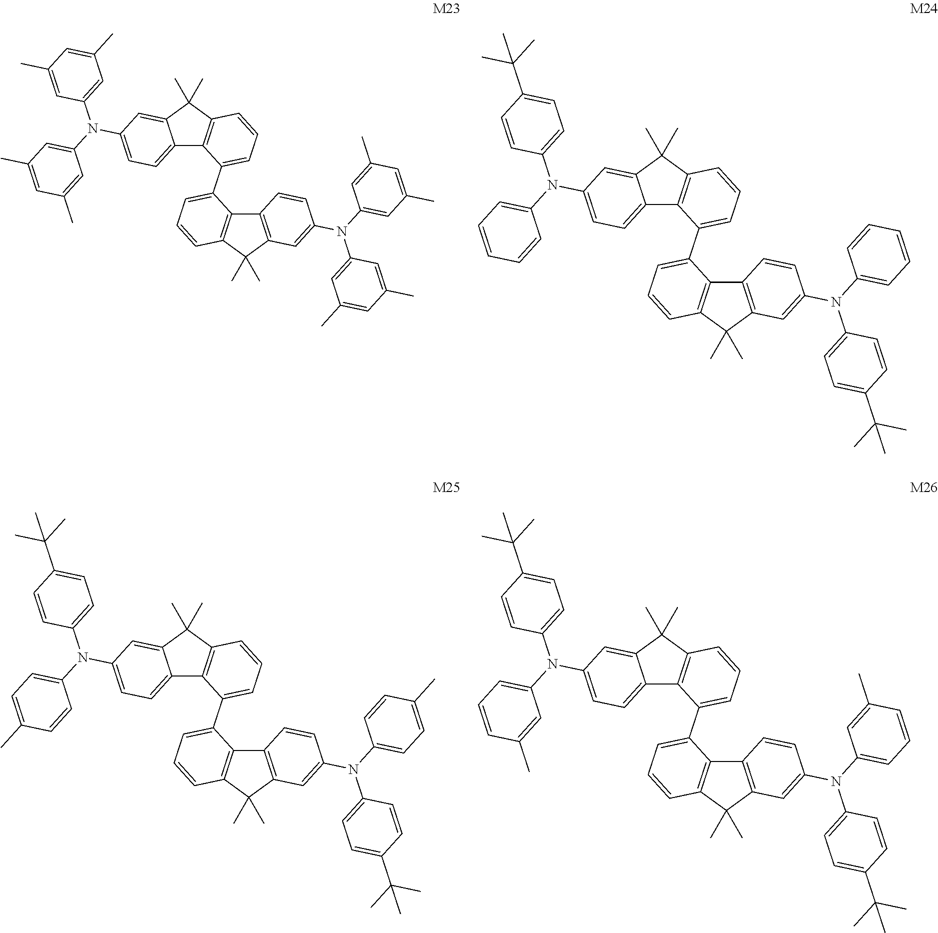

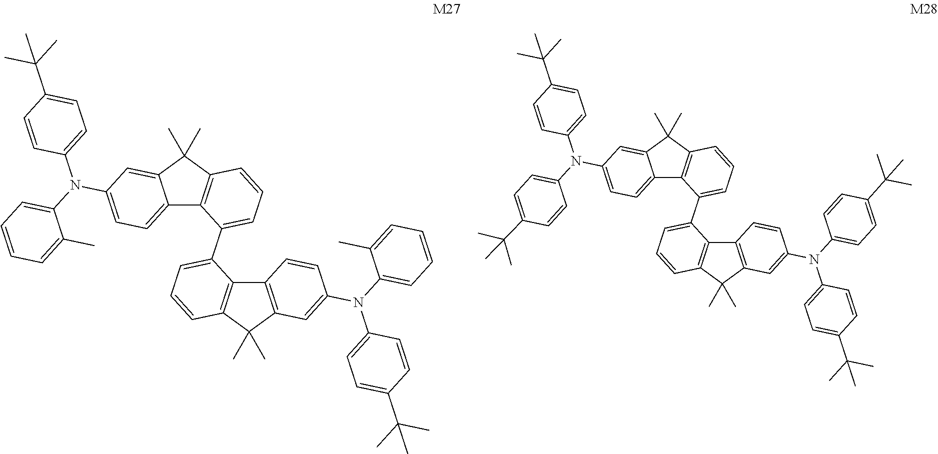 [SMILES: Cc1ccccc1N(c1ccc(C(C)(C)C)cc1)c1ccc2c(c1)C(C)(C)c1cccc(-c3cccc4c3-c3ccc(N(c5ccc(C(C)(C)C)cc5)c5ccccc5C)cc3C4(C)C)c1-2 ; CC(C)(C)c1ccc(N(c2ccc(C(C)(C)C)cc2)c2ccc3c(c2)C(C)(C)c2cccc(-c4cccc5c4-c4ccc(N(c6ccc(C(C)(C)C)cc6)c6ccc(C(C)(C)C)cc6)cc4C5(C)C)c2-3)cc1]

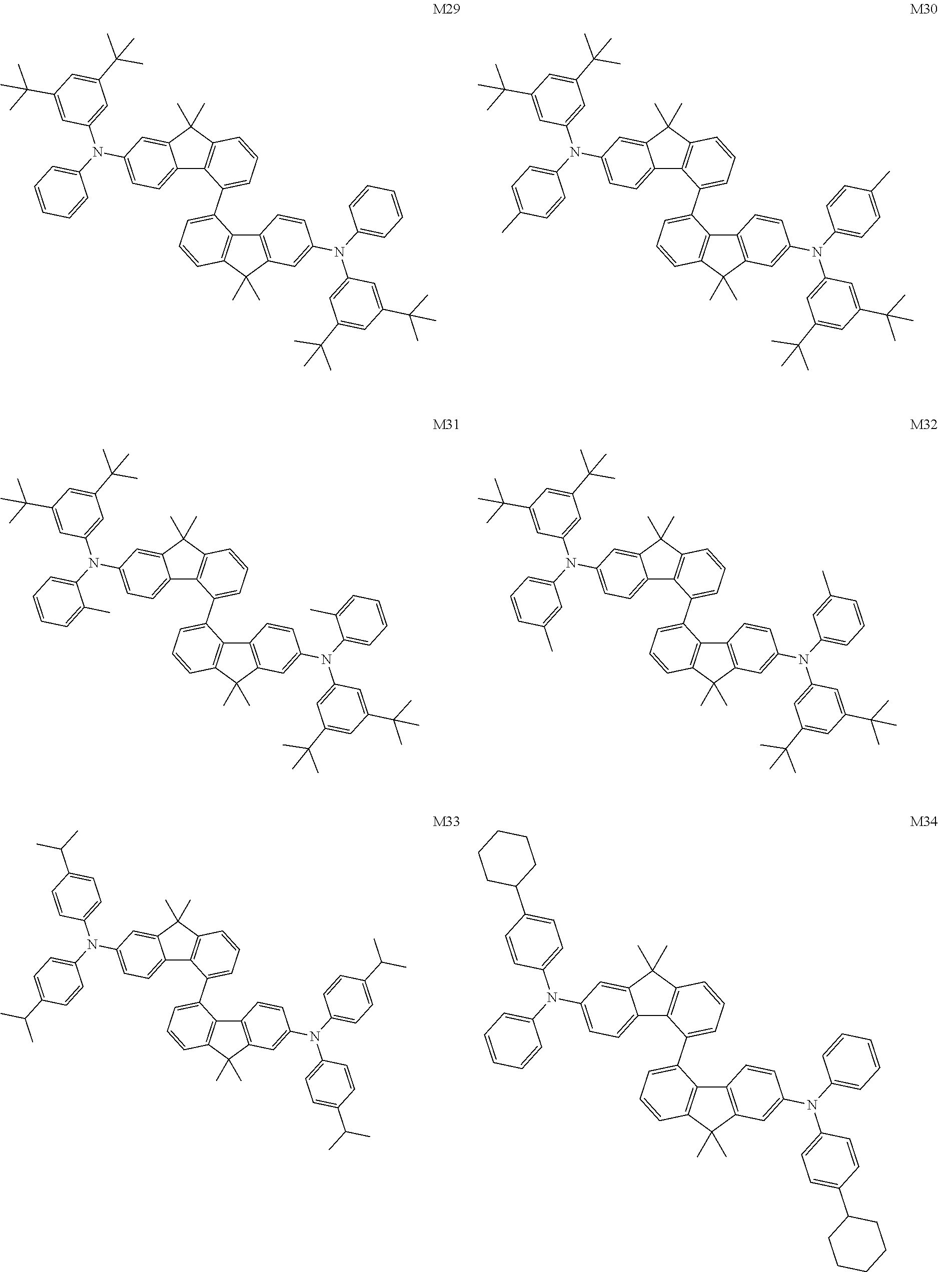

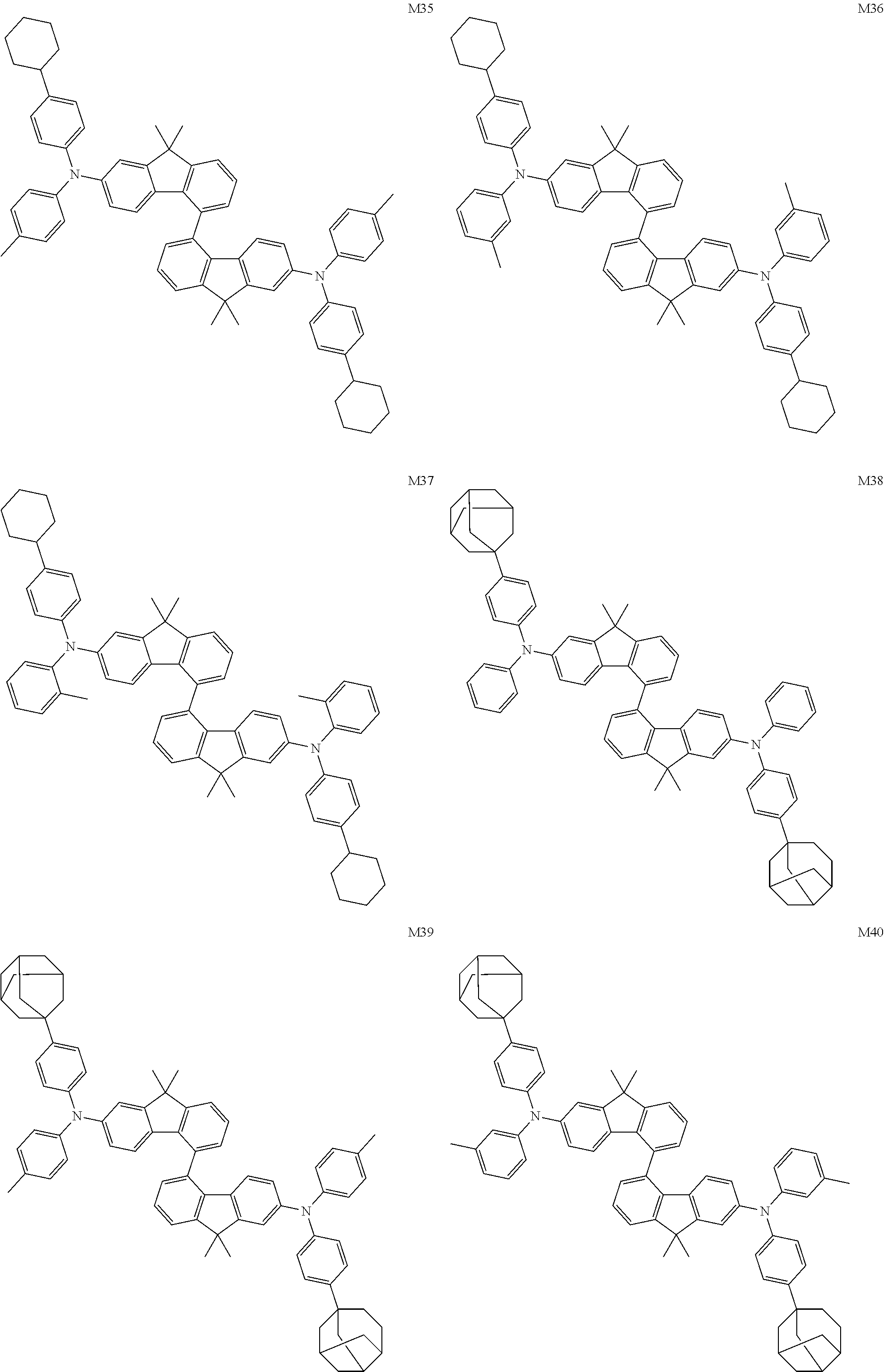

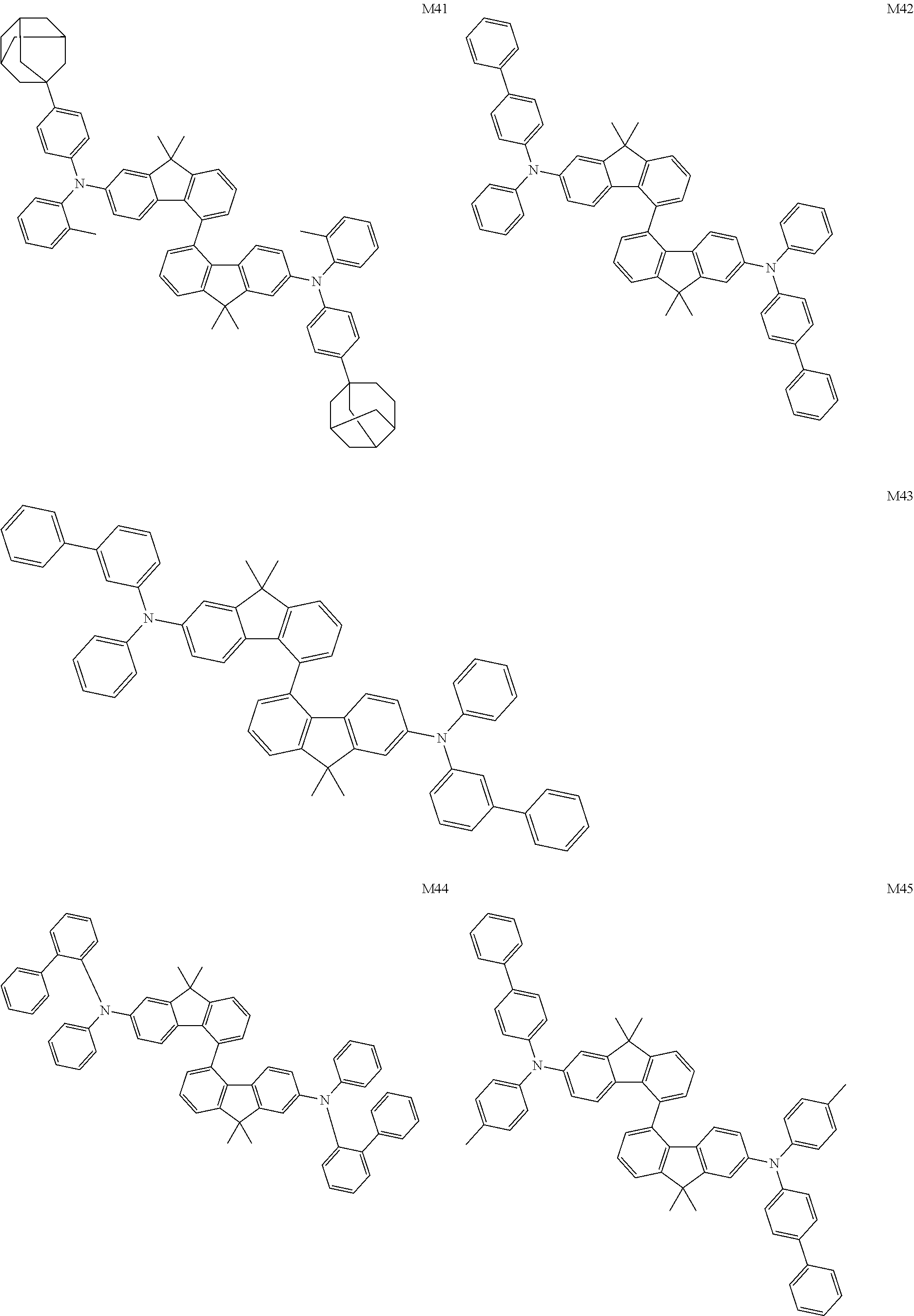

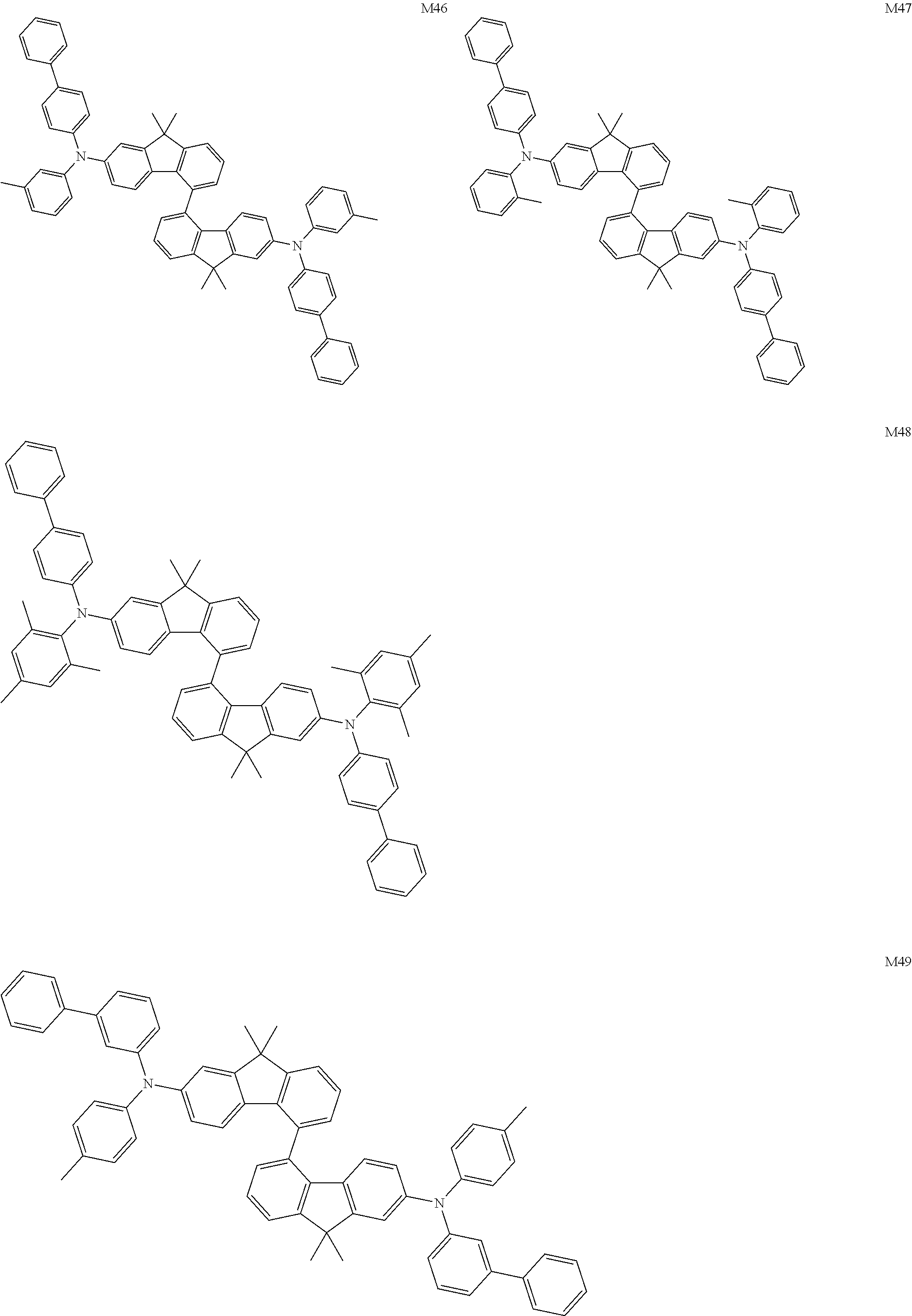

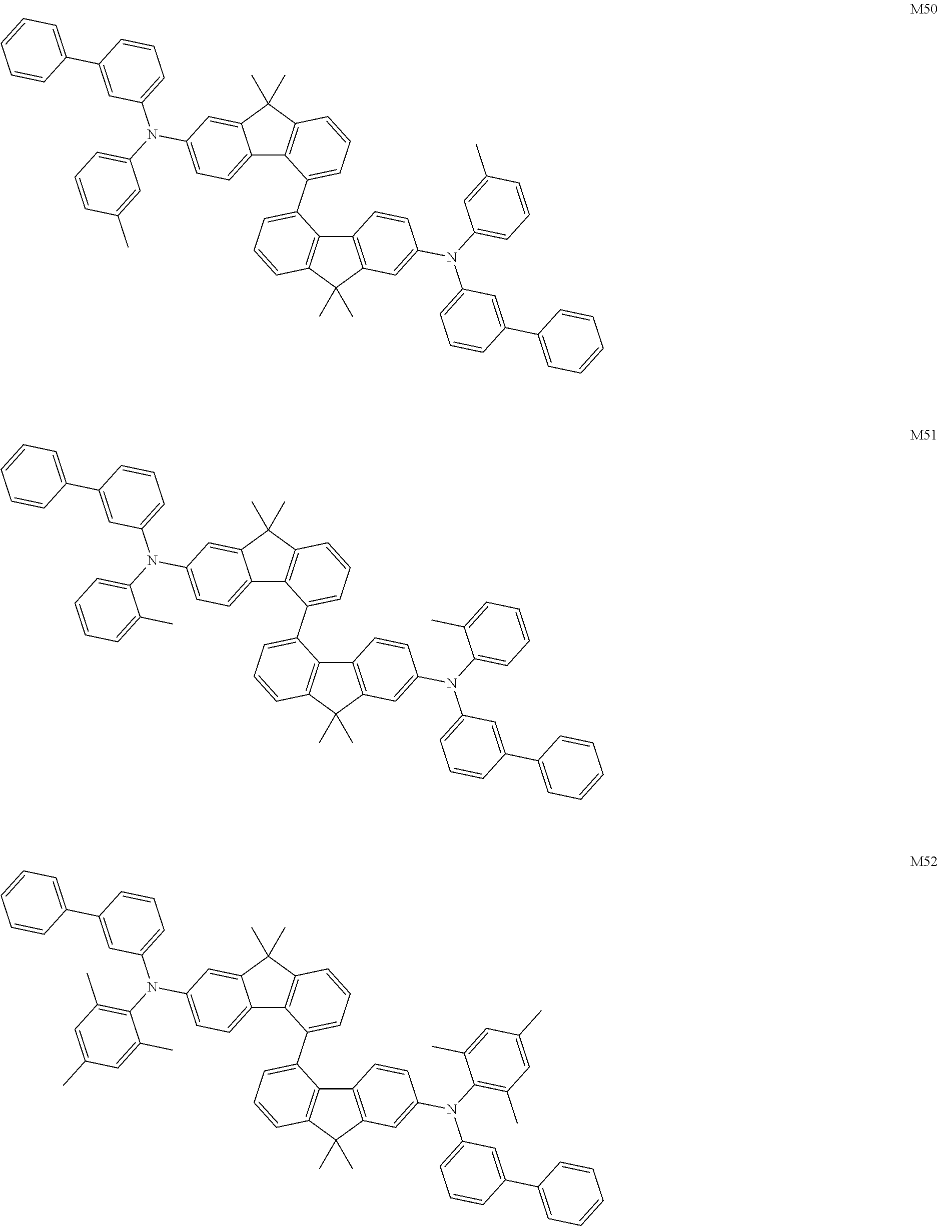 [SMILES: Cc1cccc(N(c2cccc(-c3ccccc3)c2)c2ccc3c(c2)C(C)(C)c2cccc(-c4cccc5c4-c4ccc(N(c6cccc(C)c6)c6cccc(-c7ccccc7)c6)cc4C5(C)C)c2-3)c1 ; Cc1ccccc1N(c1cccc(-c2ccccc2)c1)c1ccc2c(c1)C(C)(C)c1cccc(-c3cccc4c3-c3ccc(N(c5cccc(-c6ccccc6)c5)c5ccccc5C)cc3C4(C)C)c1-2 ; Cc1cc(C)c(N(c2cccc(-c3ccccc3)c2)c2ccc3c(c2)C(C)(C)c2cccc(-c4cccc5c4-c4ccc(N(c6cccc(-c7ccccc7)c6)c6c(C)cc(C)cc6C)cc4C5(C)C)c2-3)c(C)c1]

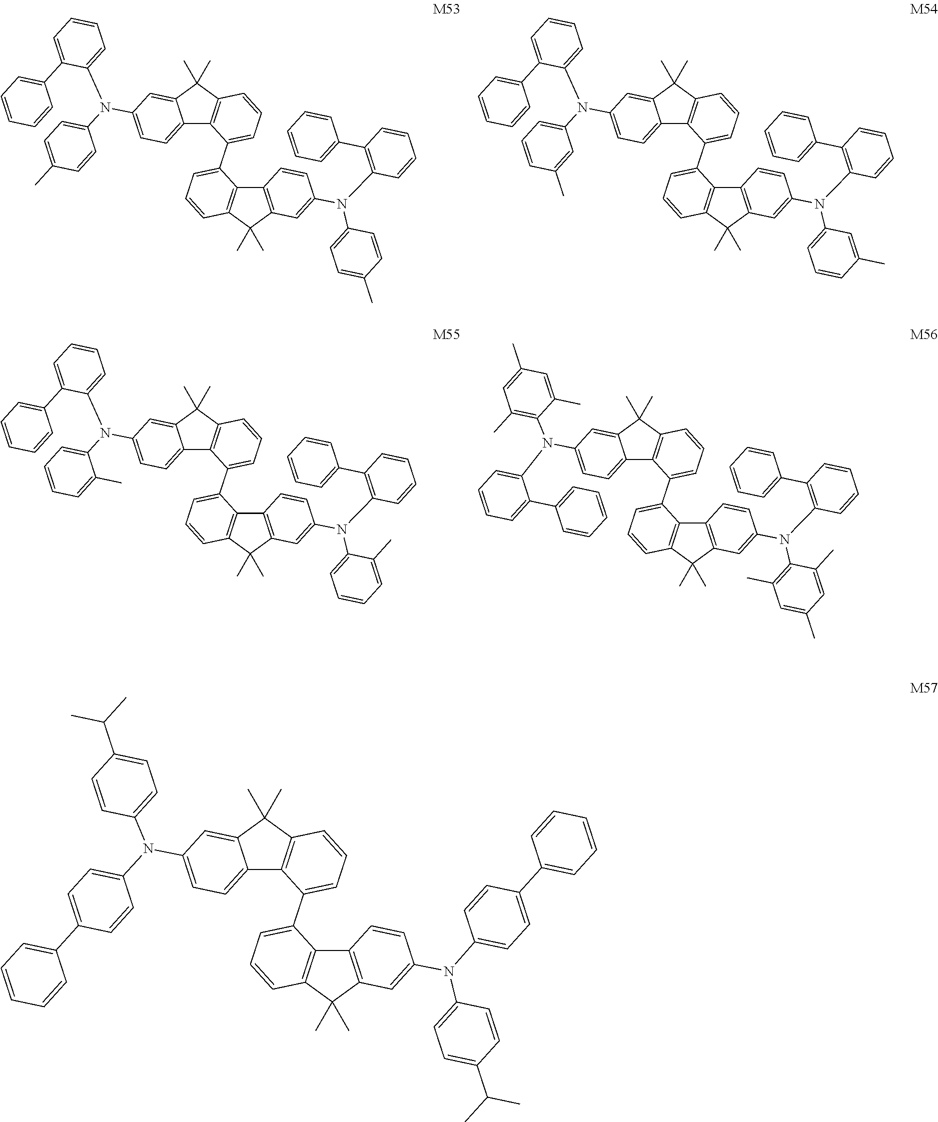

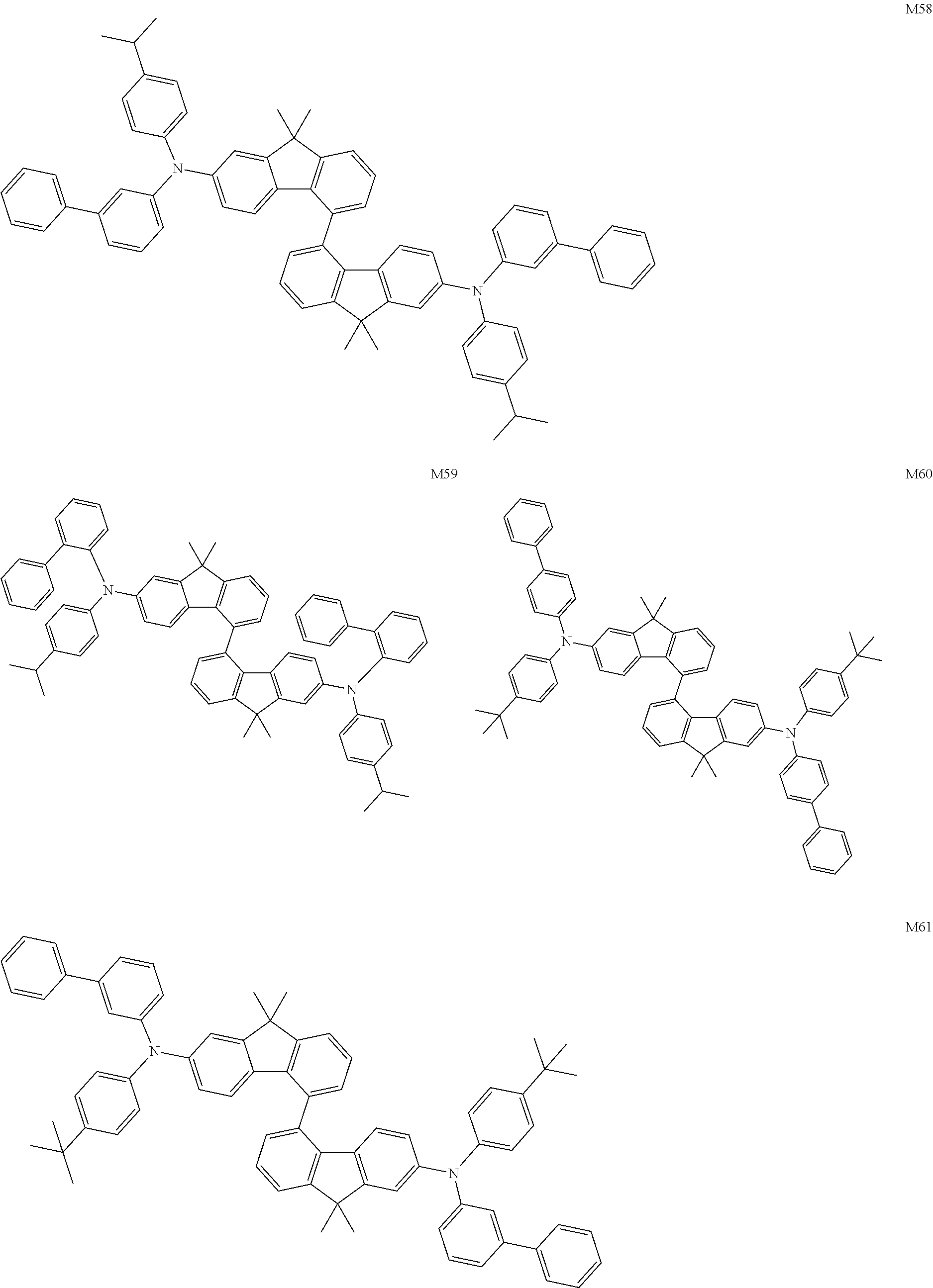

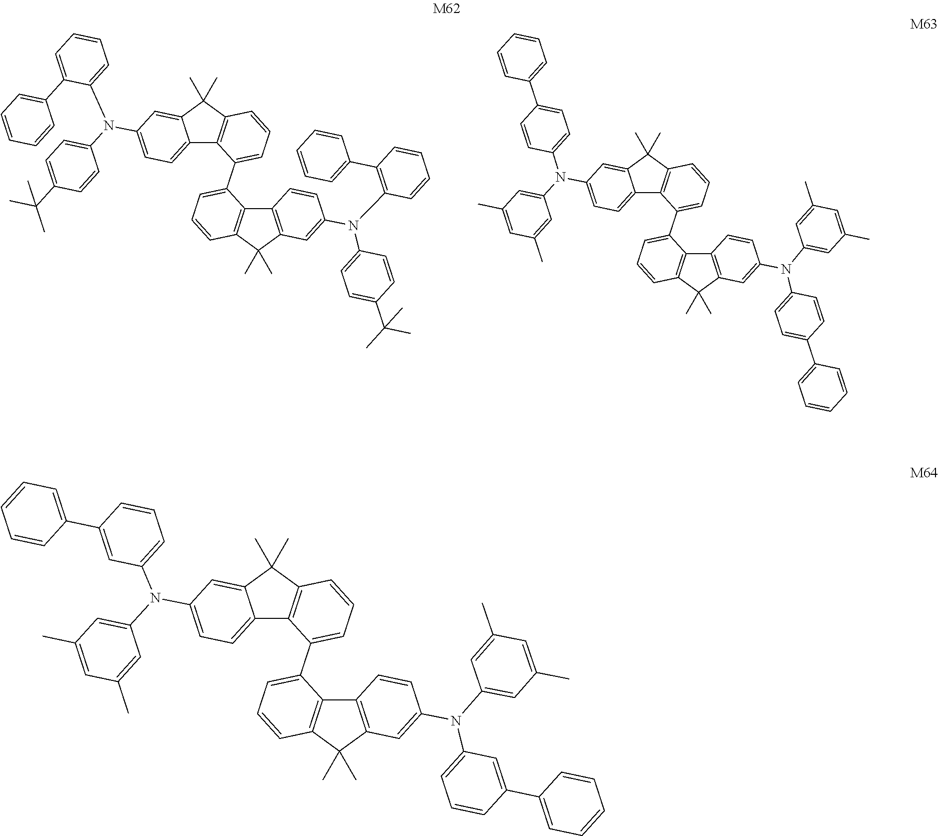

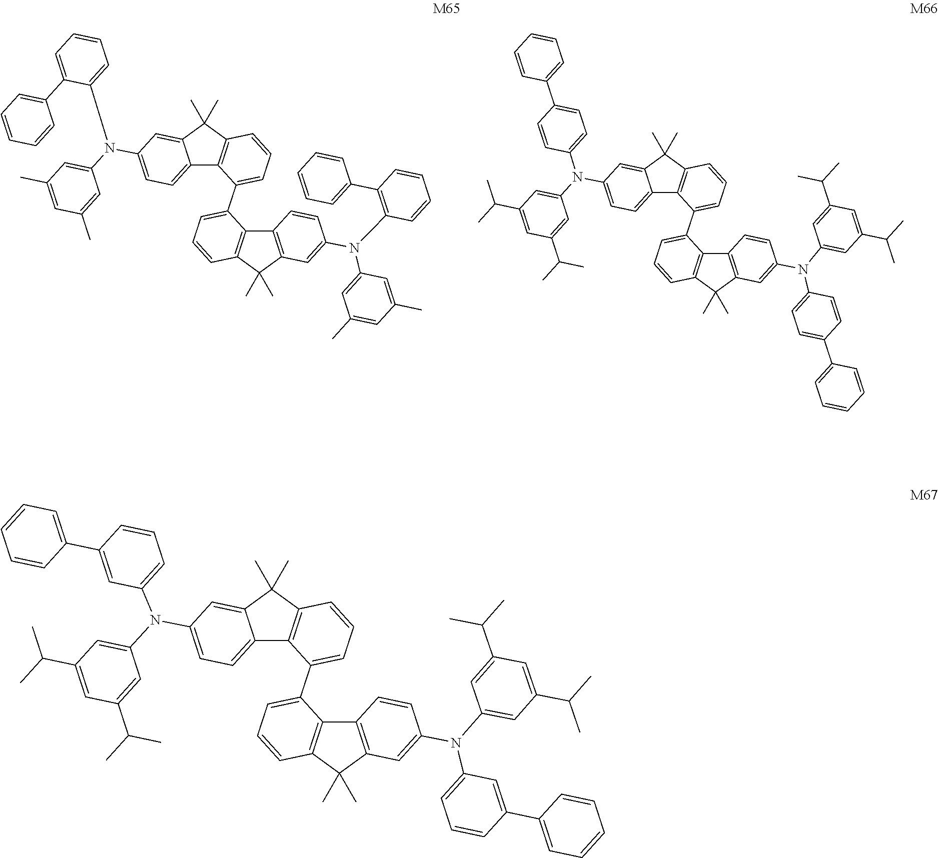 [SMILES: Cc1cc(C)cc(N(c2ccc3c(c2)C(C)(C)c2cccc(-c4cccc5c4-c4ccc(N(c6cc(C)cc(C)c6)c6ccccc6-c6ccccc6)cc4C5(C)C)c2-3)c2ccccc2-c2ccccc2)c1 ; CC(C)c1cc(C(C)C)cc(N(c2ccc(-c3ccccc3)cc2)c2ccc3c(c2)C(C)(C)c2cccc(-c4cccc5c4-c4ccc(N(c6ccc(-c7ccccc7)cc6)c6cc(C(C)C)cc(C(C)C)c6)cc4C5(C)C)c2-3)c1 ; CC(C)c1cc(C(C)C)cc(N(c2cccc(-c3ccccc3)c2)c2ccc3c(c2)C(C)(C)c2cccc(-c4cccc5c4-c4ccc(N(c6cccc(-c7ccccc7)c6)c6cc(C(C)C)cc(C(C)C)c6)cc4C5(C)C)c2-3)c1]

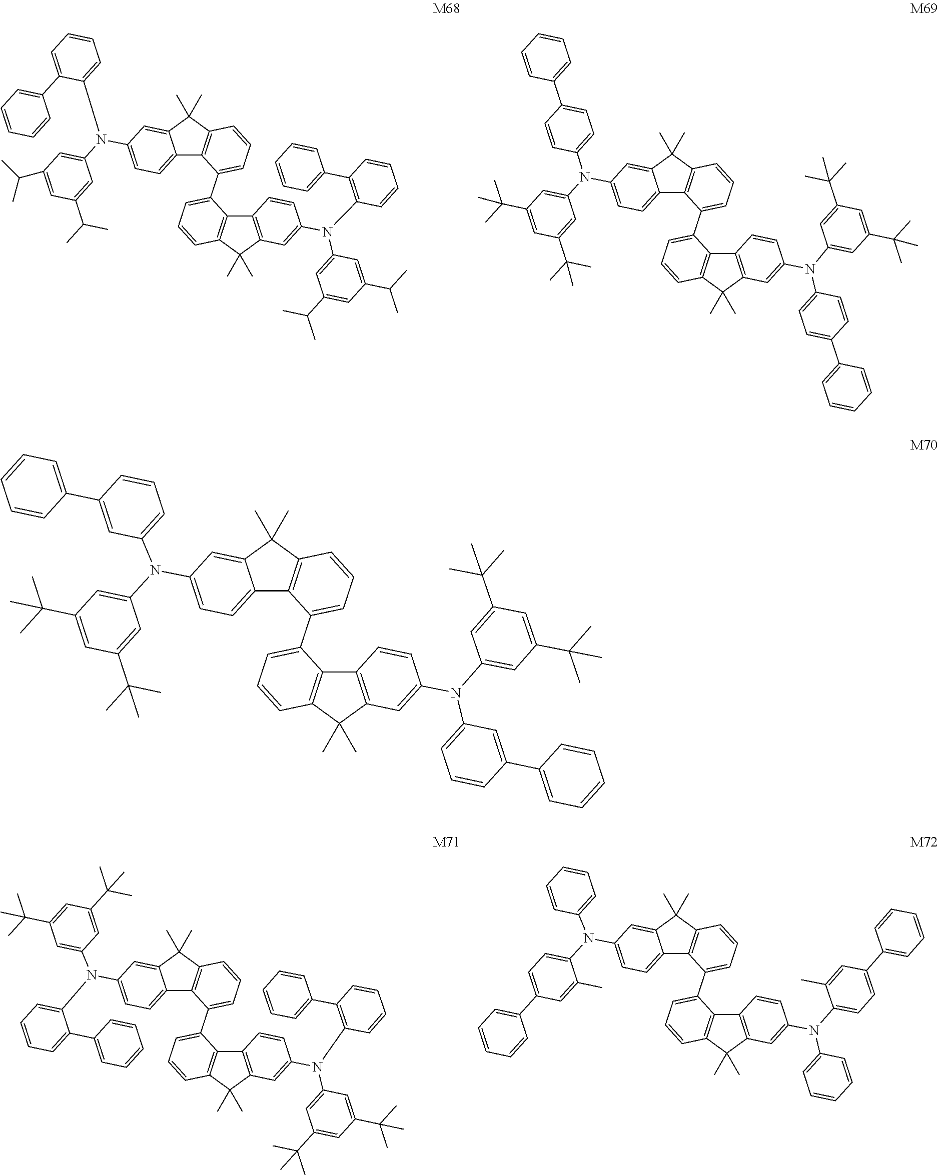

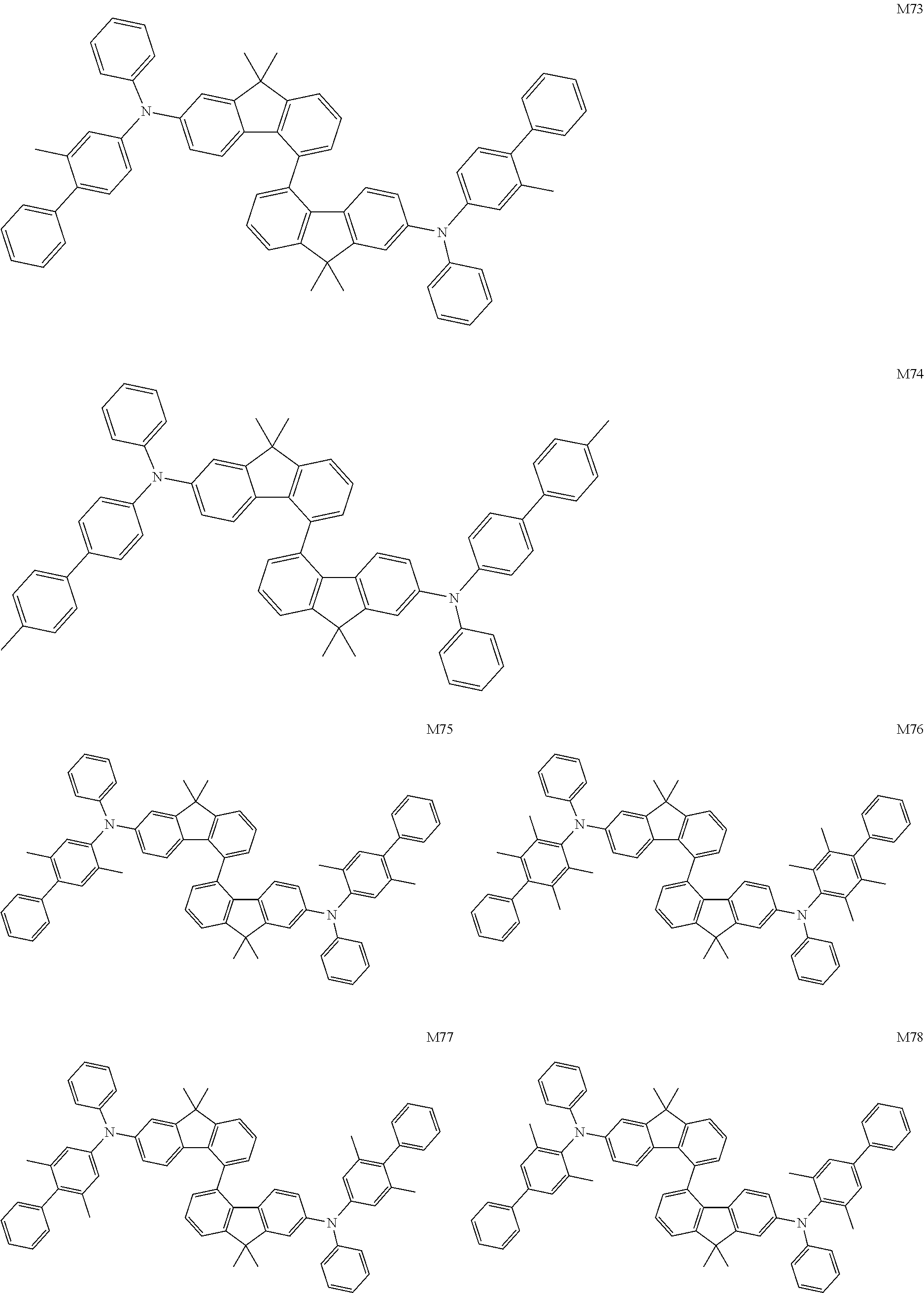

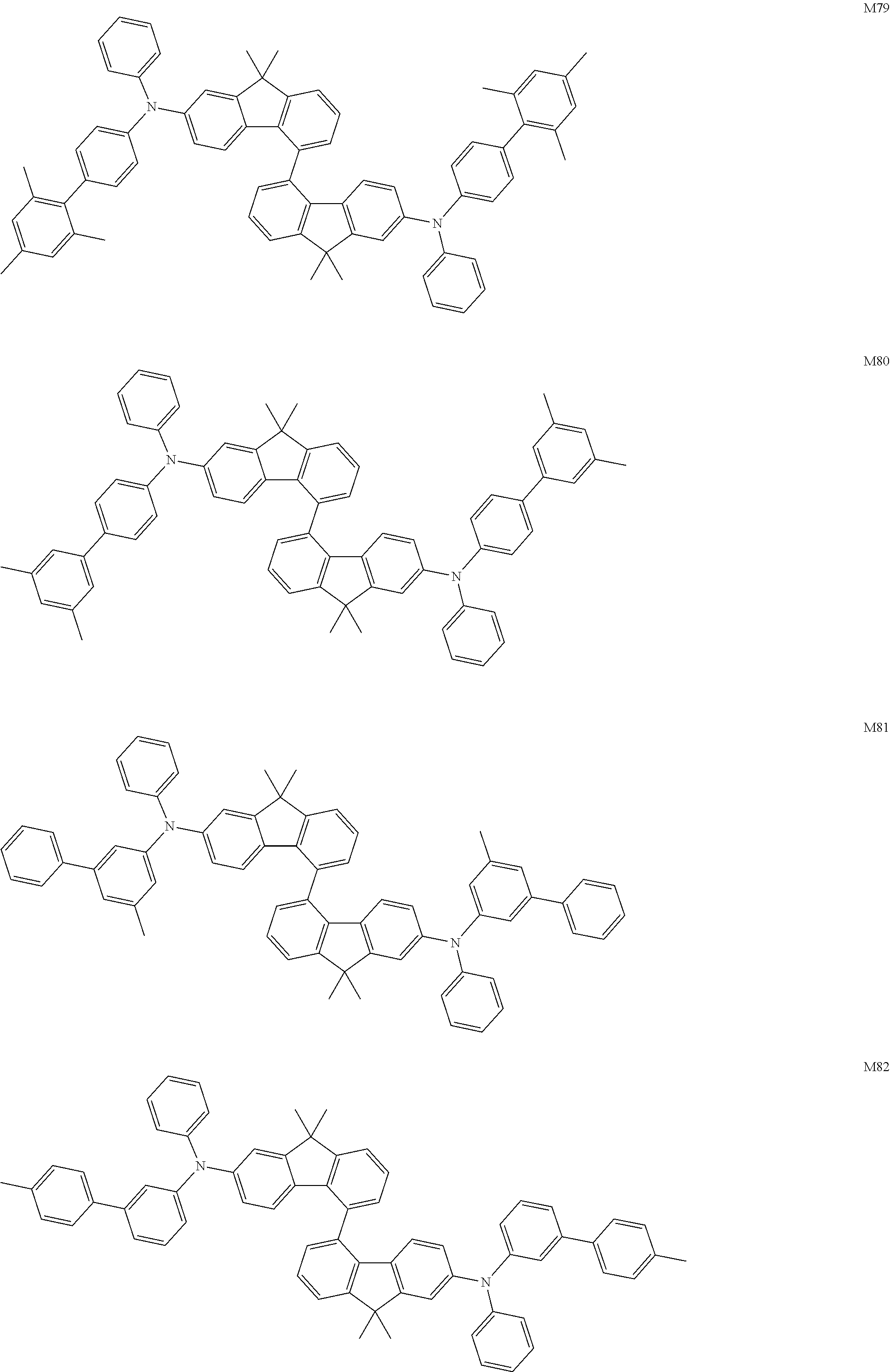

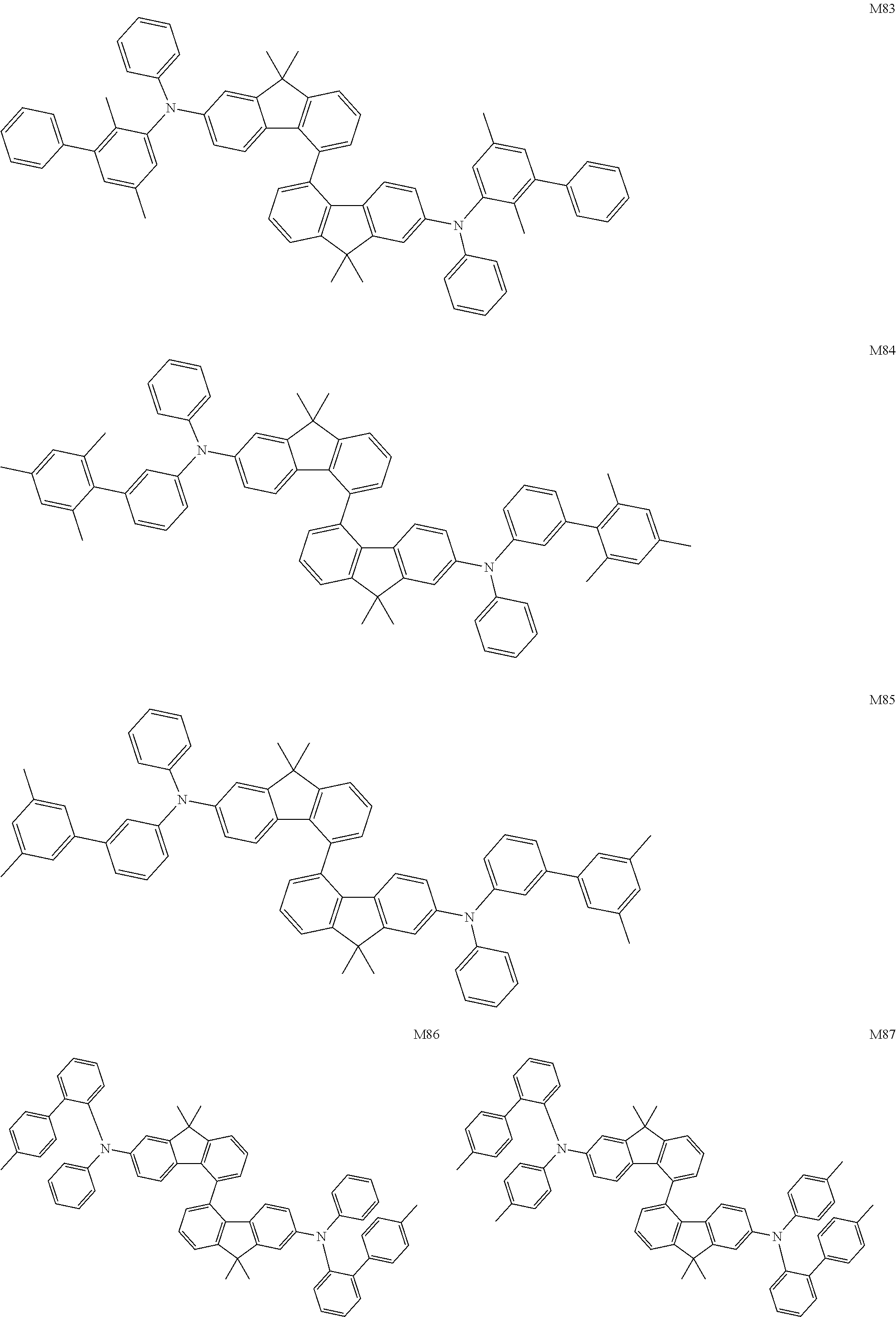

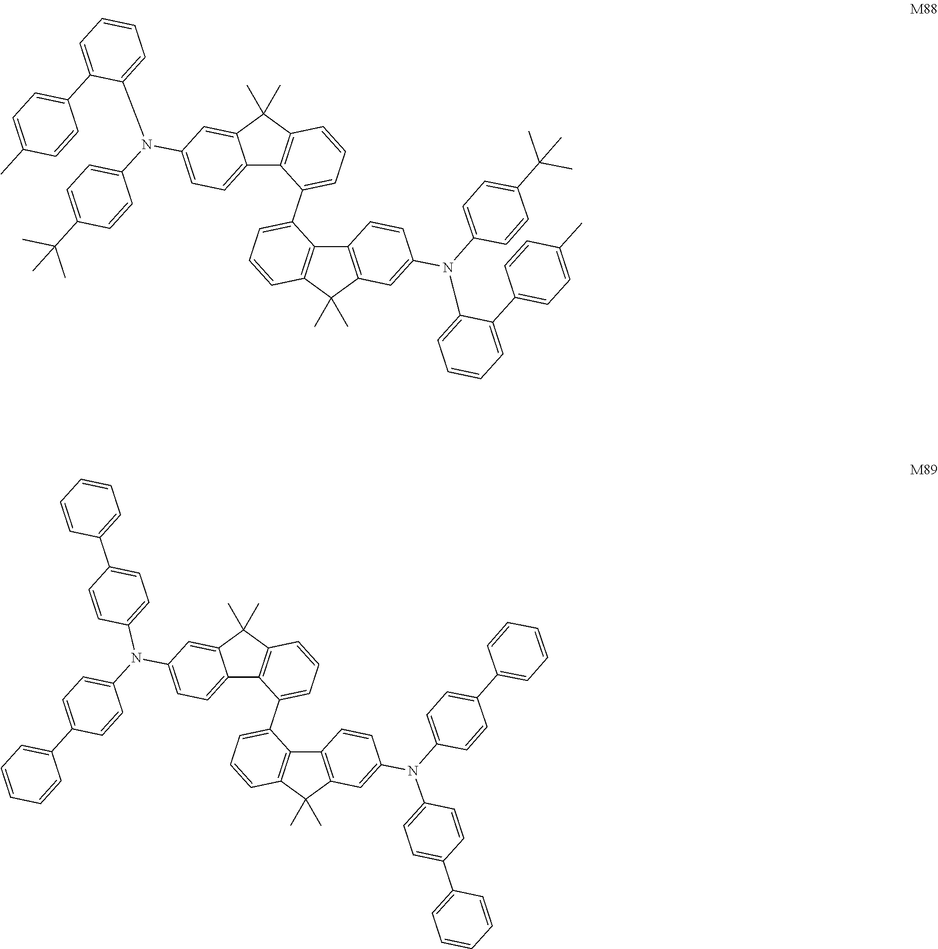

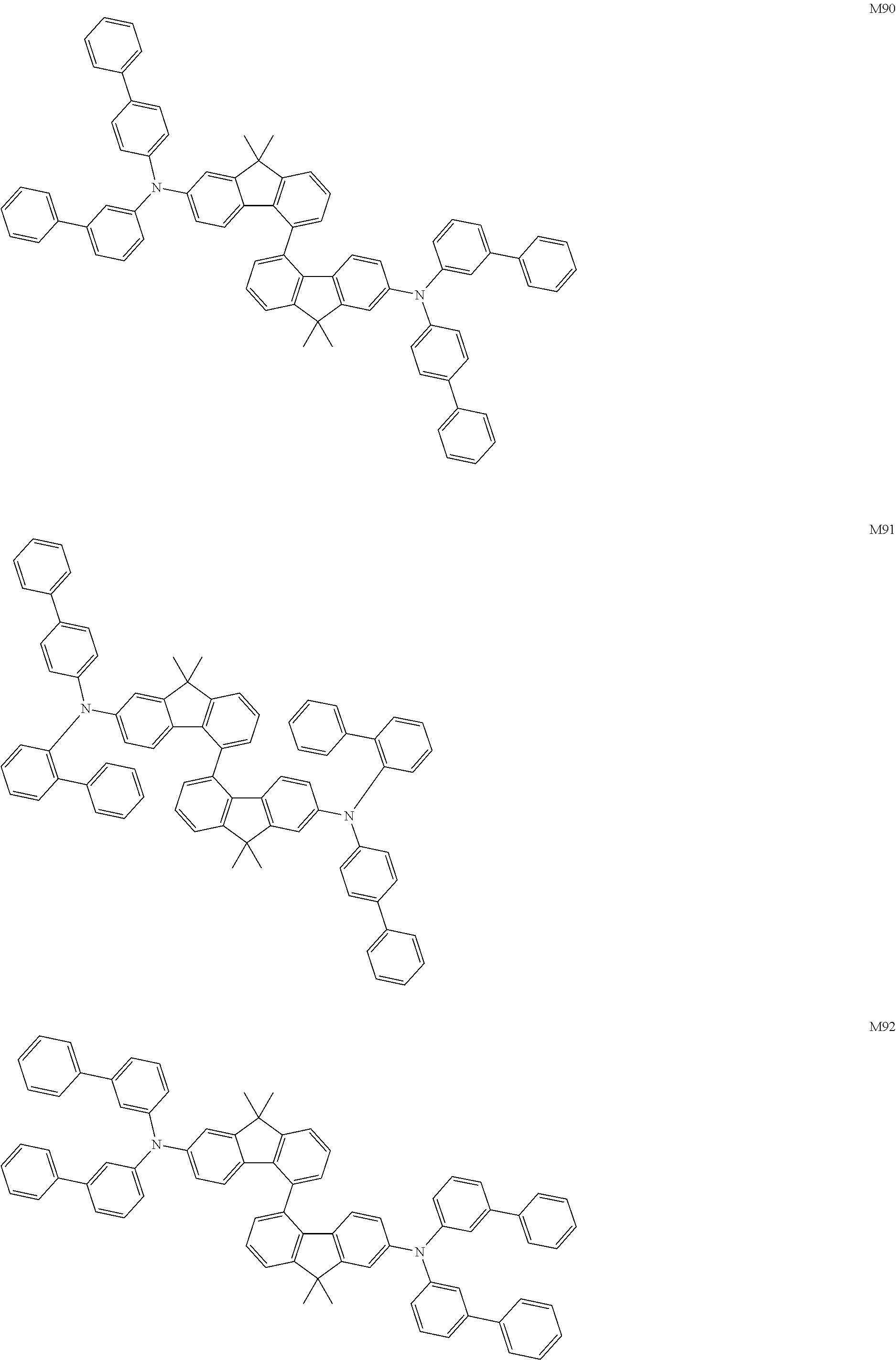 [SMILES: CC1(C)c2cc(N(c3ccc(-c4ccccc4)cc3)c3cccc(-c4ccccc4)c3)ccc2-c2c(-c3cccc4c3-c3ccc(N(c5ccc(-c6ccccc6)cc5)c5cccc(-c6ccccc6)c5)cc3C4(C)C)cccc21 ; CC1(C)c2cc(N(c3ccc(-c4ccccc4)cc3)c3ccccc3-c3ccccc3)ccc2-c2c(-c3cccc4c3-c3ccc(N(c5ccc(-c6ccccc6)cc5)c5ccccc5-c5ccccc5)cc3C4(C)C)cccc21 ; CC1(C)c2cc(N(c3cccc(-c4ccccc4)c3)c3cccc(-c4ccccc4)c3)ccc2-c2c(-c3cccc4c3-c3ccc(N(c5cccc(-c6ccccc6)c5)c5cccc(-c6ccccc6)c5)cc3C4(C)C)cccc21]

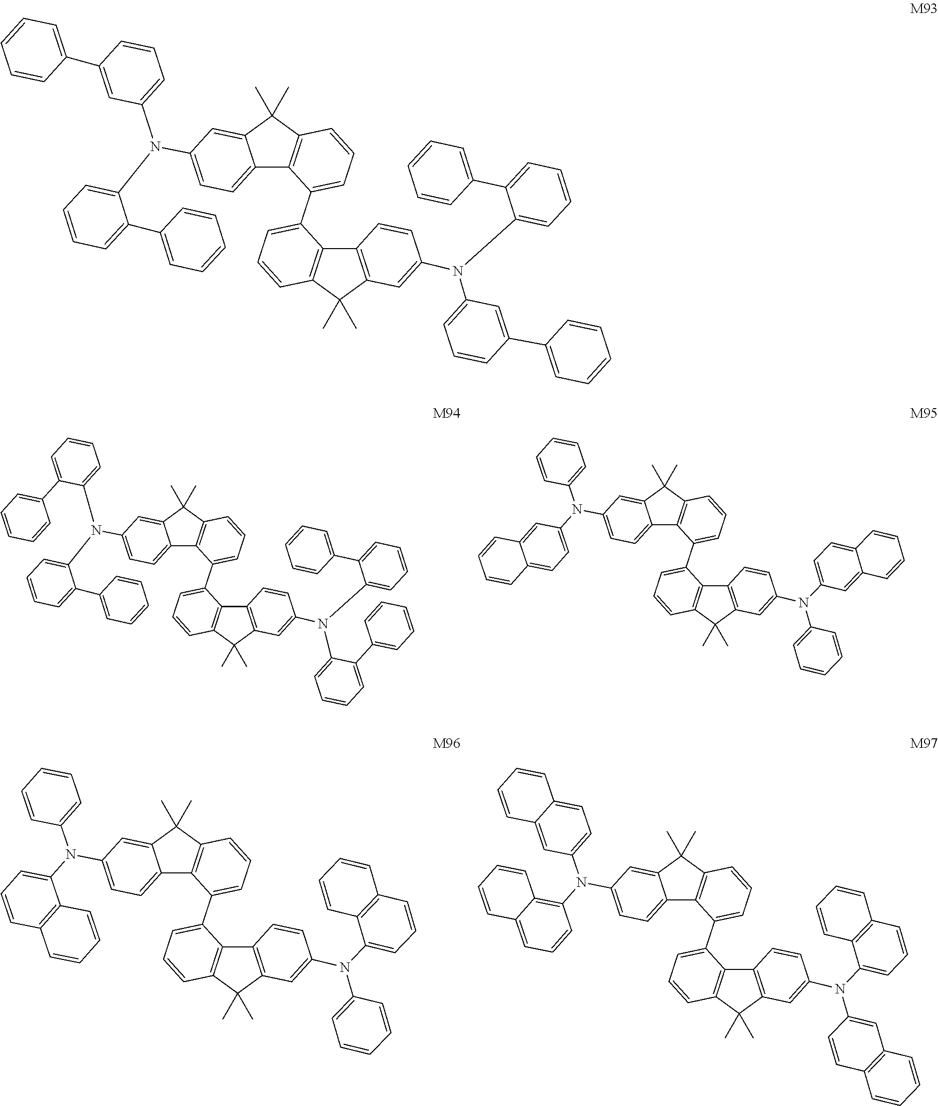

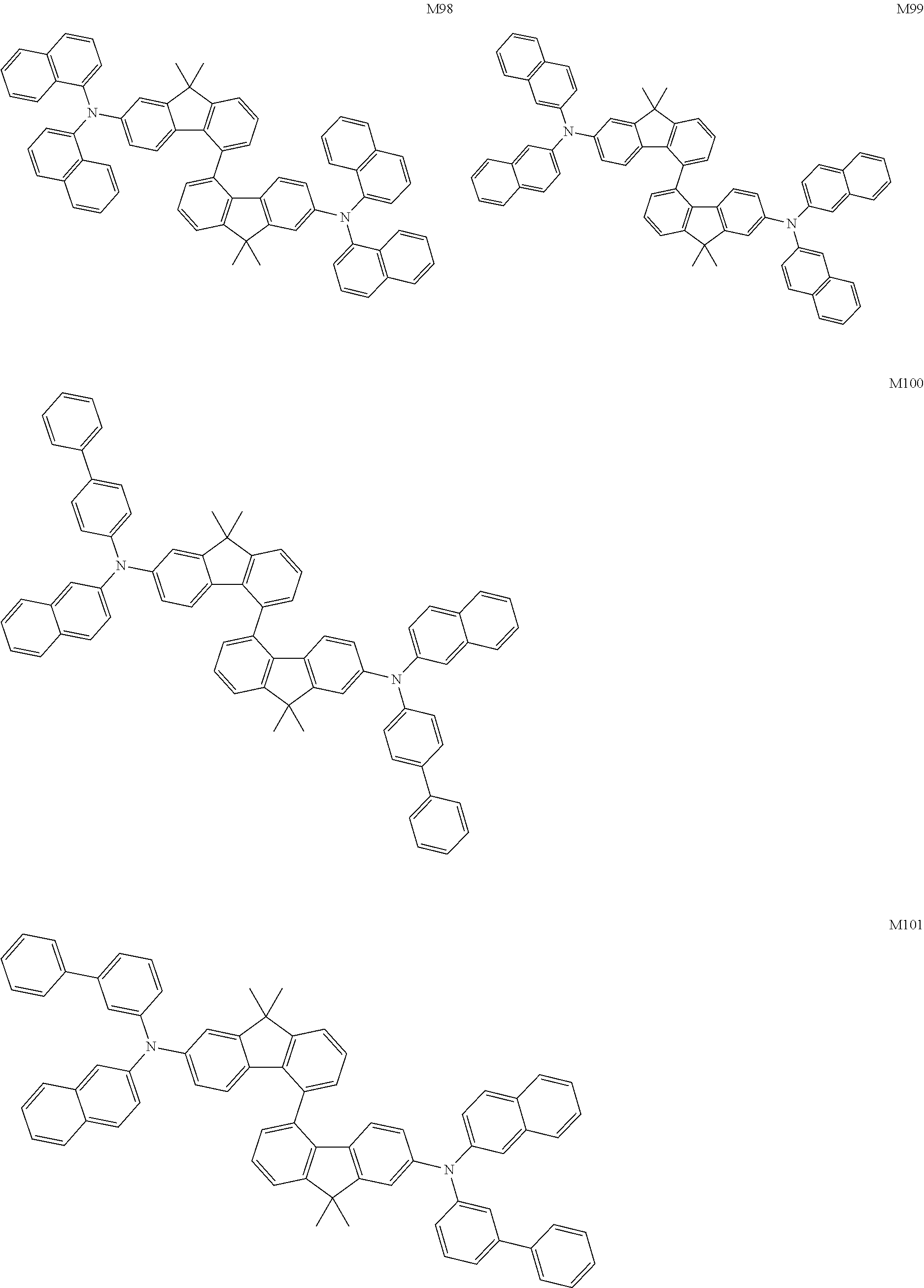 [SMILES: CC1(C)c2cc(N(c3cccc4ccccc34)c3cccc4ccccc34)ccc2-c2c(-c3cccc4c3-c3ccc(N(c5cccc6ccccc56)c5cccc6ccccc56)cc3C4(C)C)cccc21 ; CC1(C)c2cc(N(c3ccc4ccccc4c3)c3ccc4ccccc4c3)ccc2-c2c(-c3cccc4c3-c3ccc(N(c5ccc6ccccc6c5)c5ccc6ccccc6c5)cc3C4(C)C)cccc21 ; CC1(C)c2cc(N(c3ccc(-c4ccccc4)cc3)c3ccc4ccccc4c3)ccc2-c2c(-c3cccc4c3-c3ccc(N(c5ccc(-c6ccccc6)cc5)c5ccc6ccccc6c5)cc3C4(C)C)cccc21 ; CC1(C)c2cc(N(c3cccc(-c4ccccc4)c3)c3ccc4ccccc4c3)ccc2-c2c(-c3cccc4c3-c3ccc(N(c5cccc(-c6ccccc6)c5)c5ccc6ccccc6c5)cc3C4(C)C)cccc21]

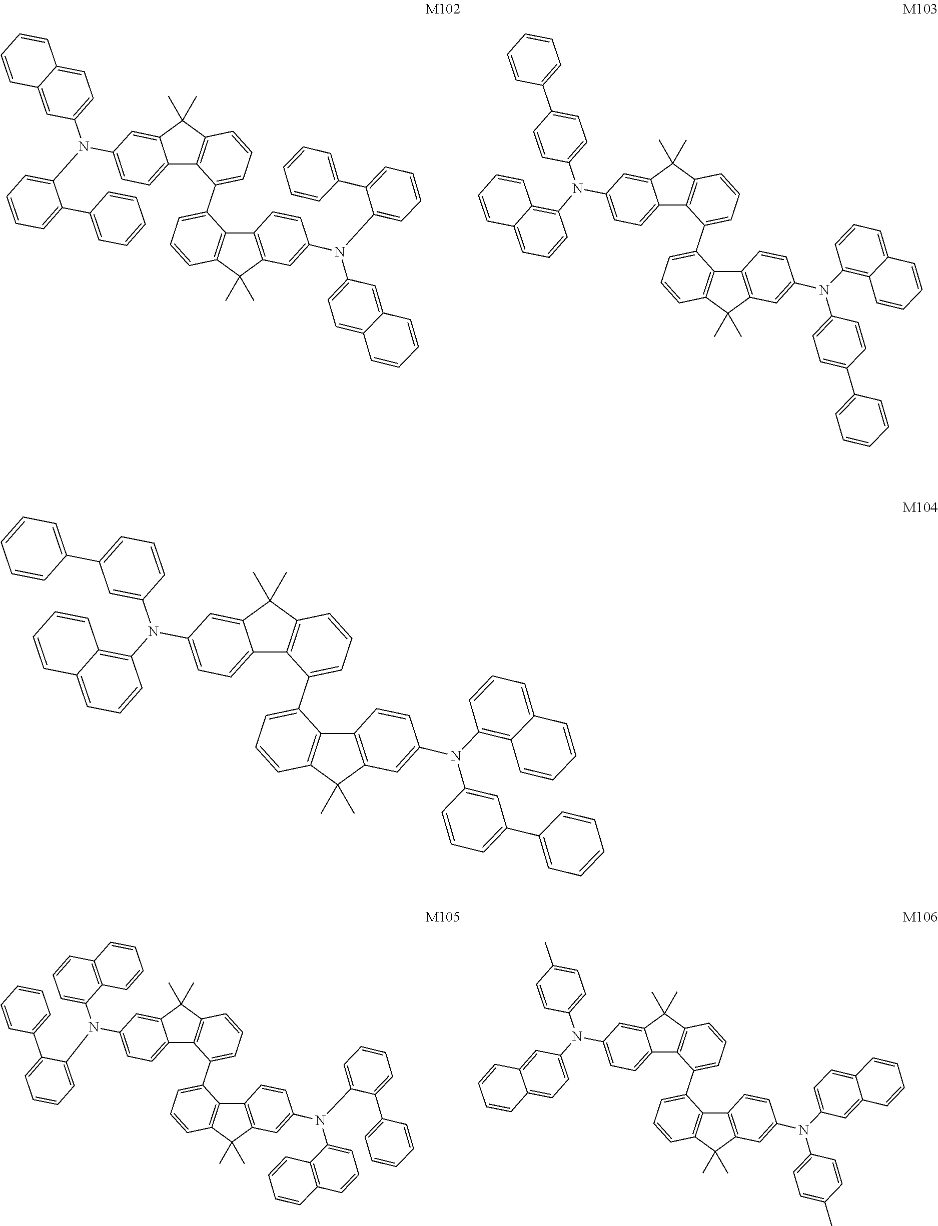

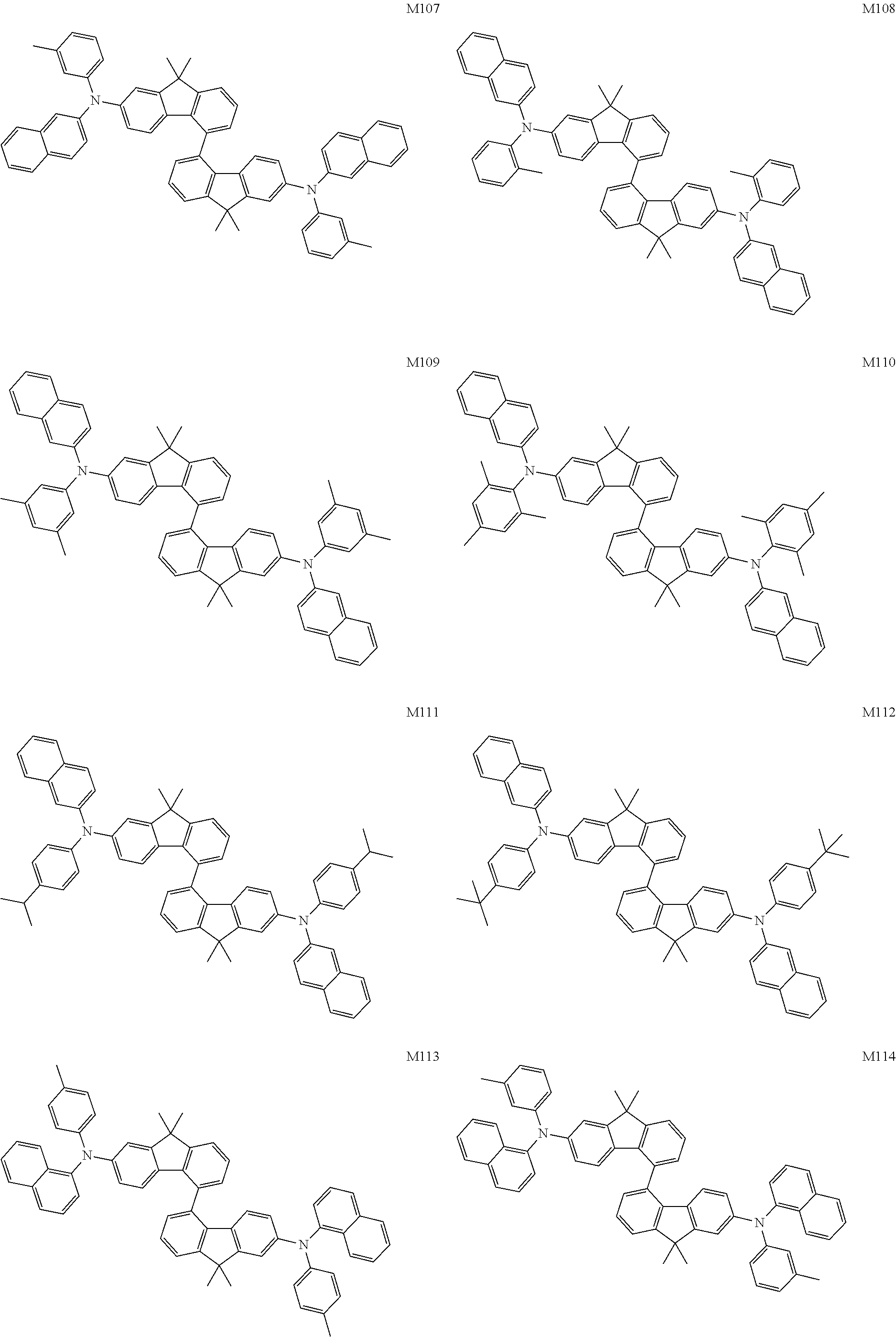

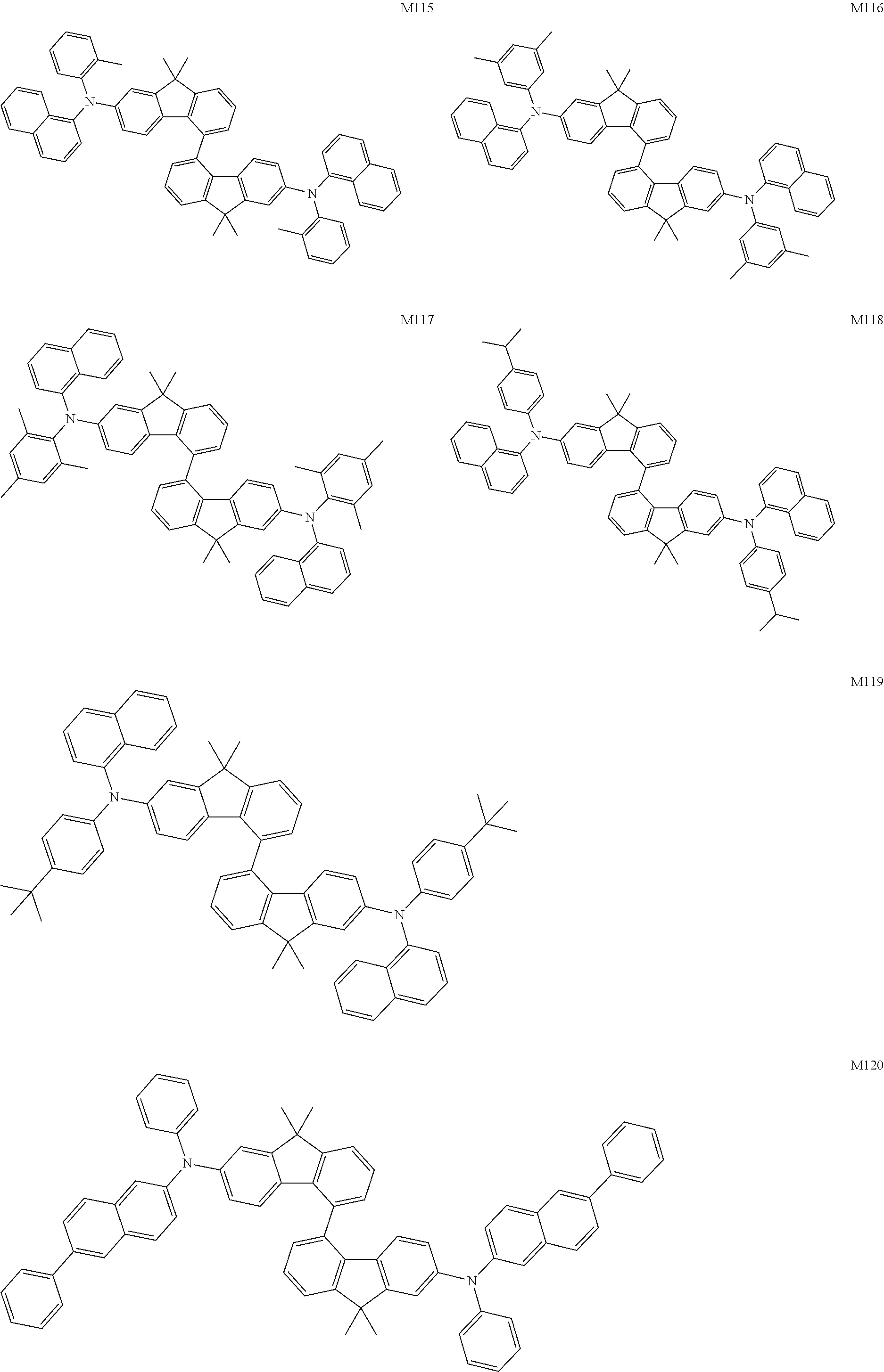

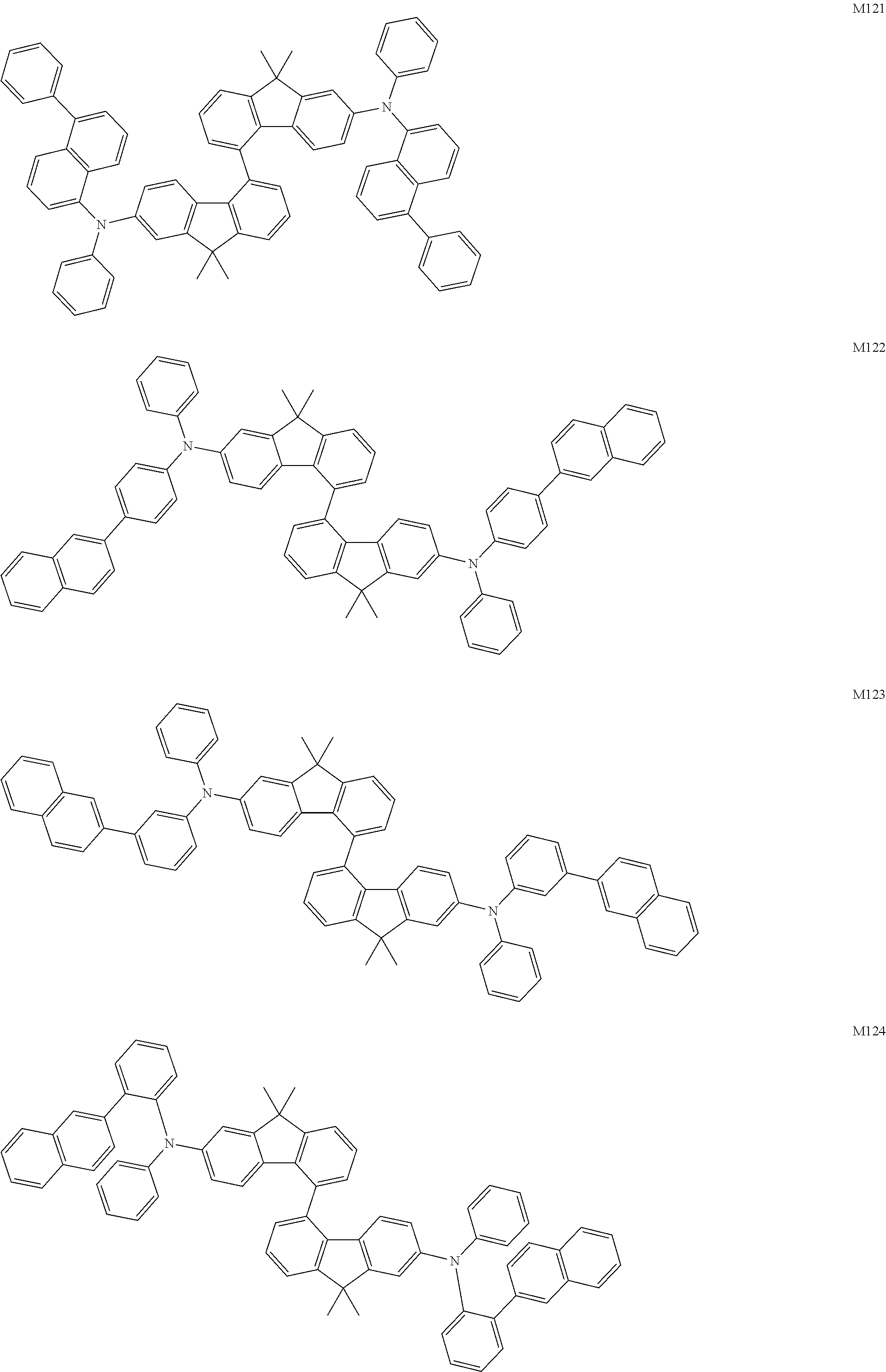

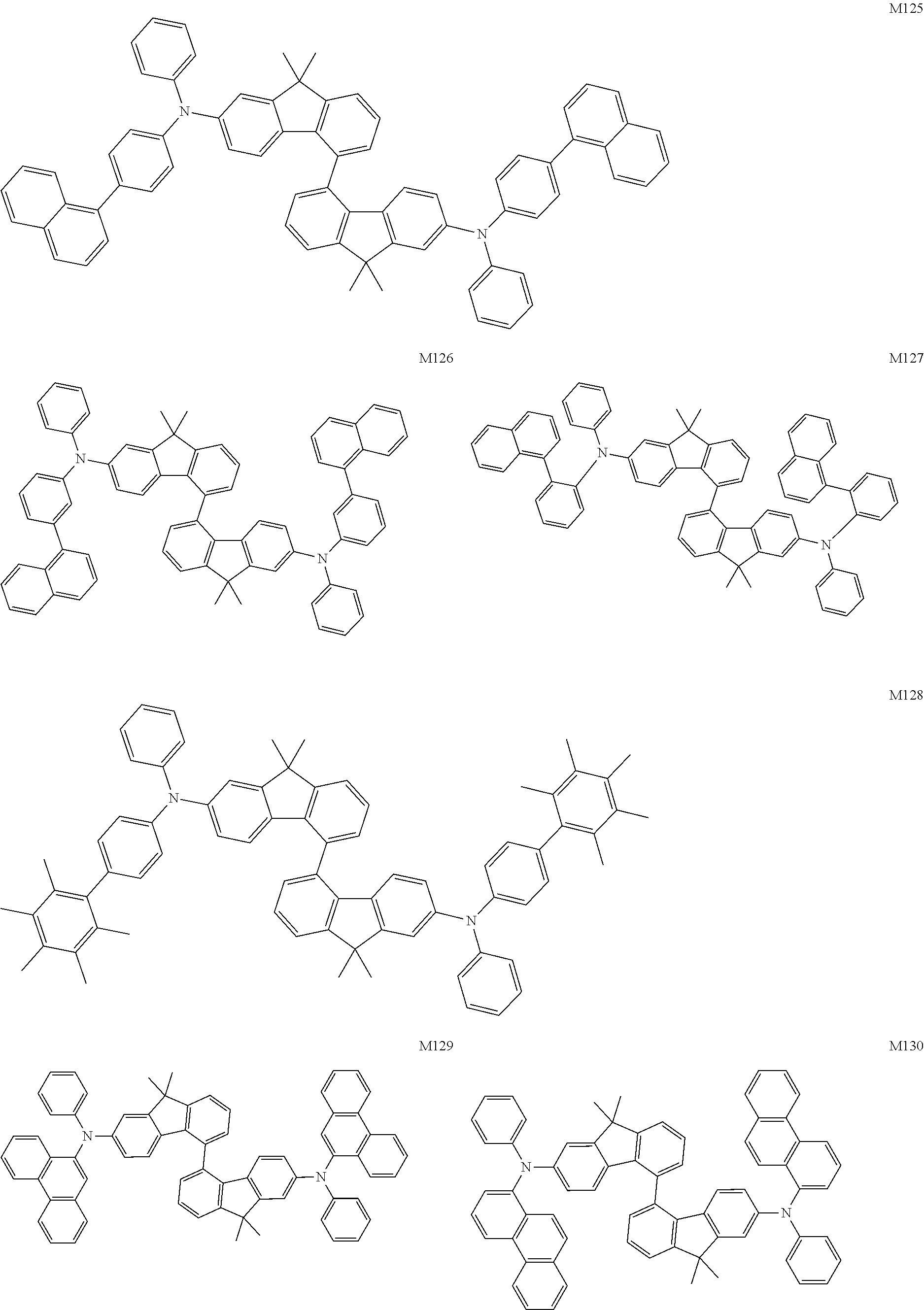

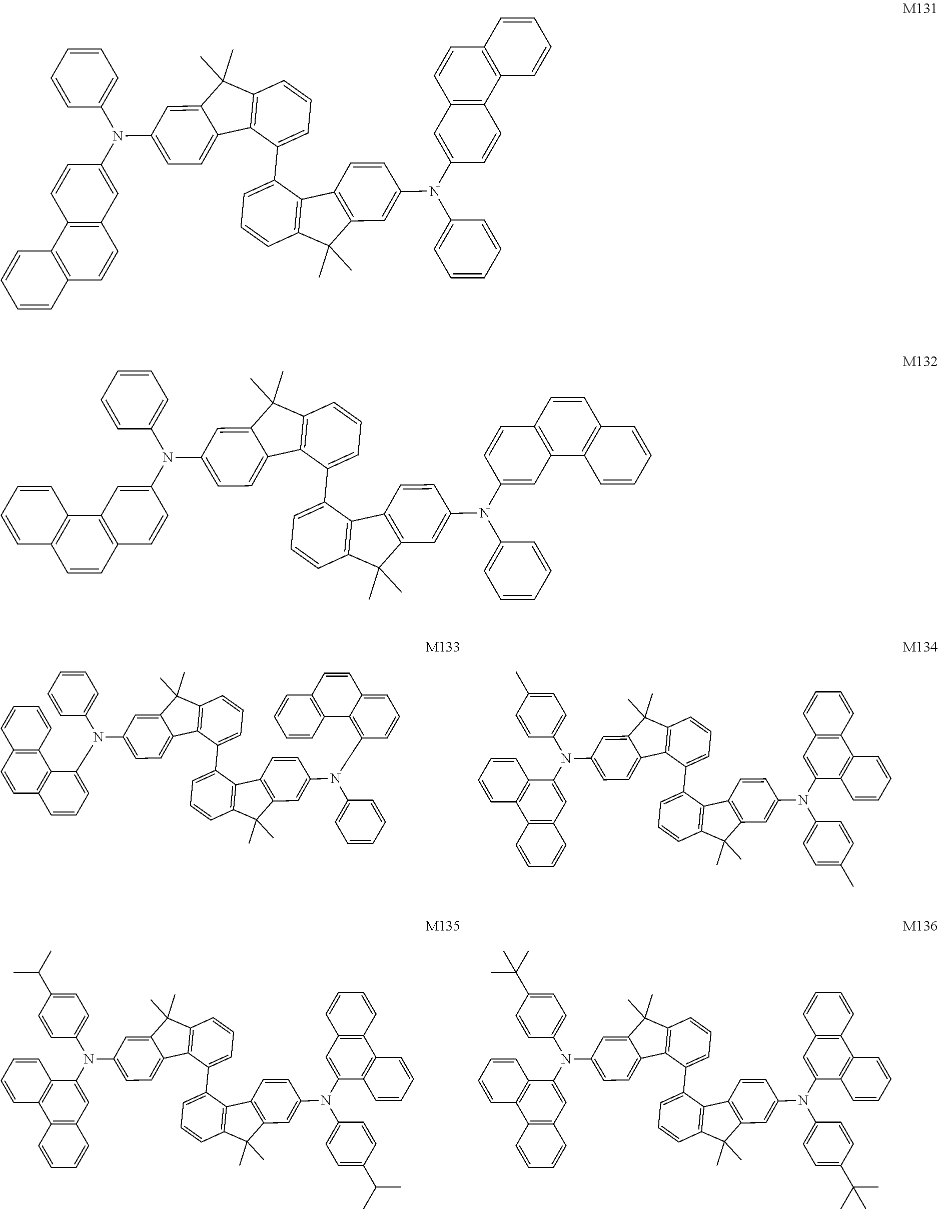

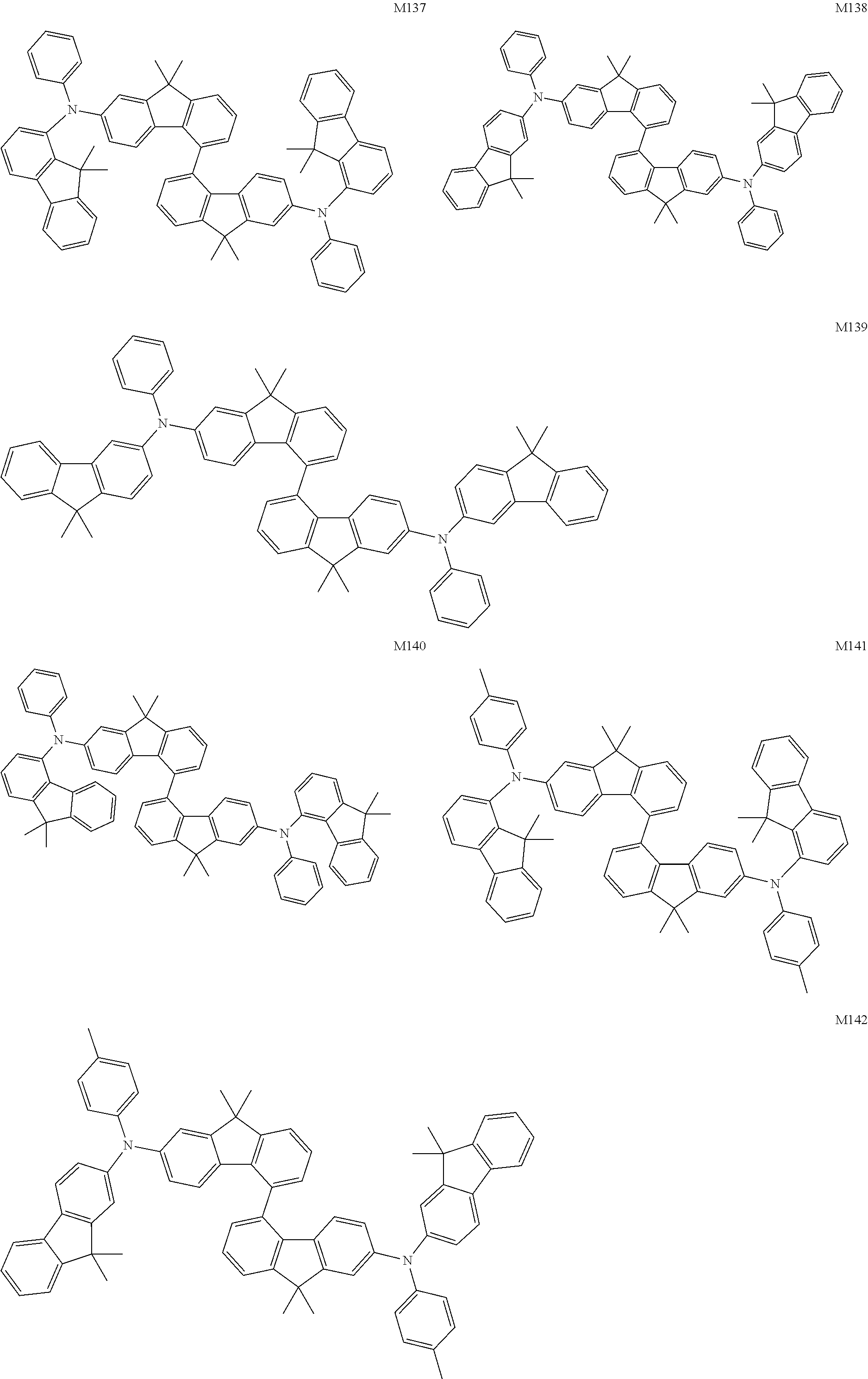

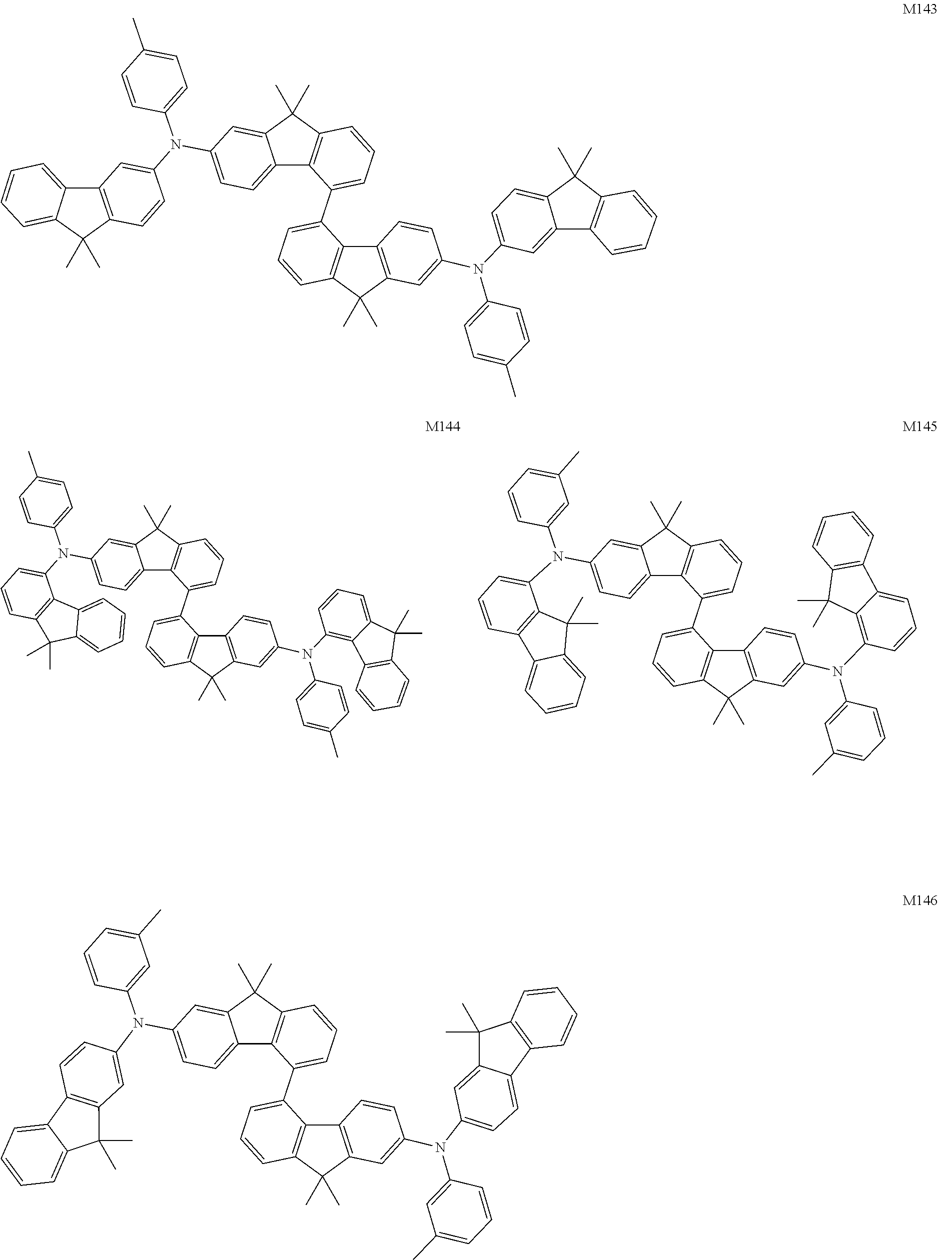

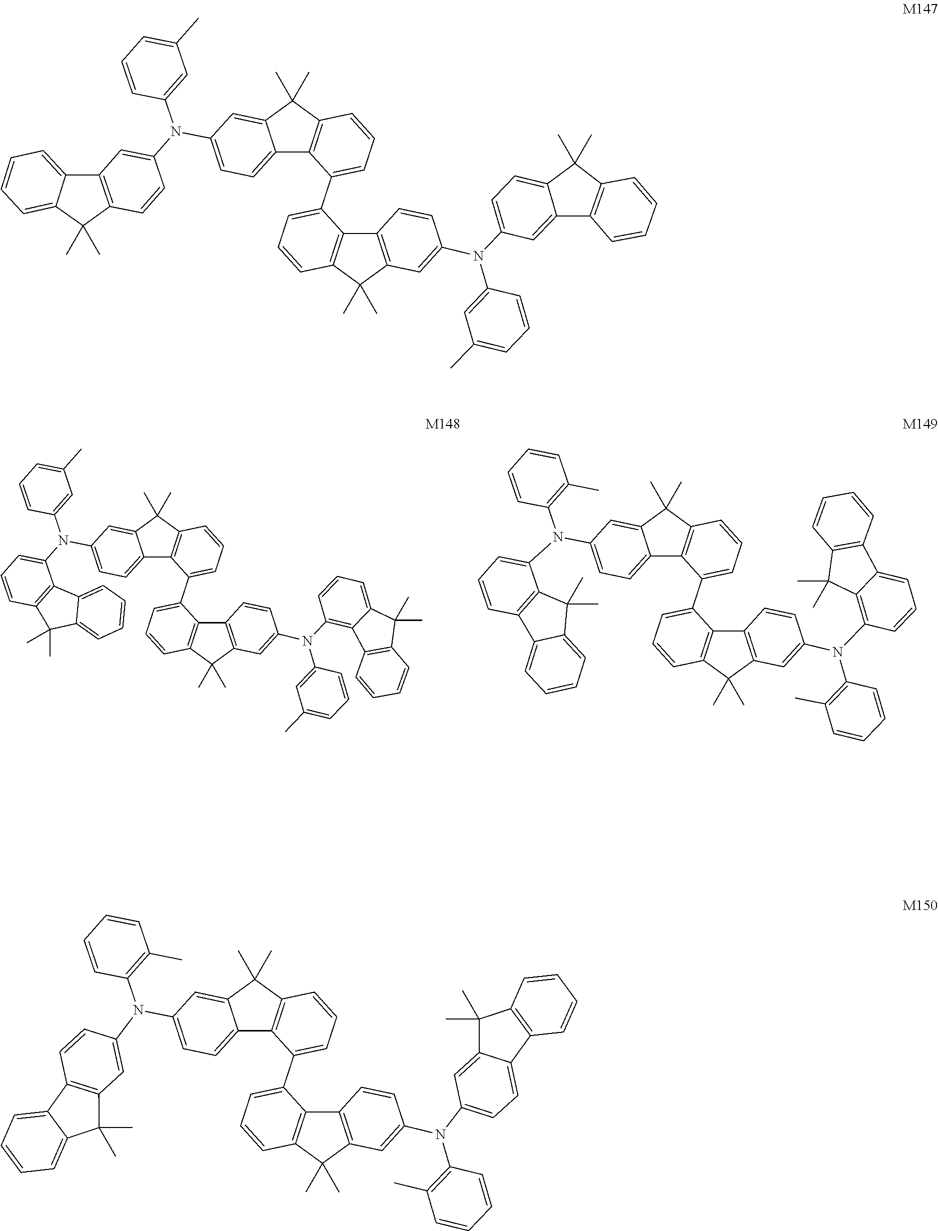

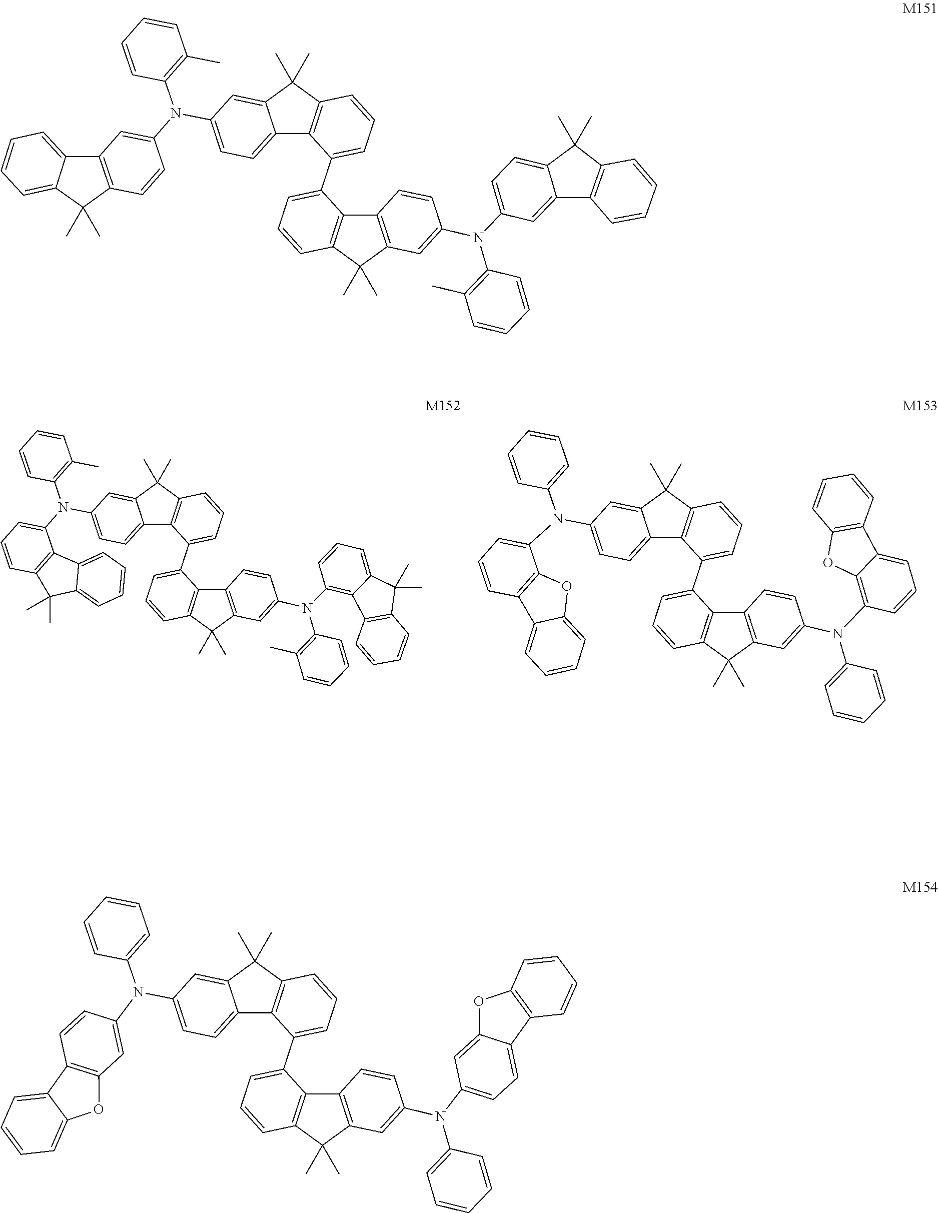

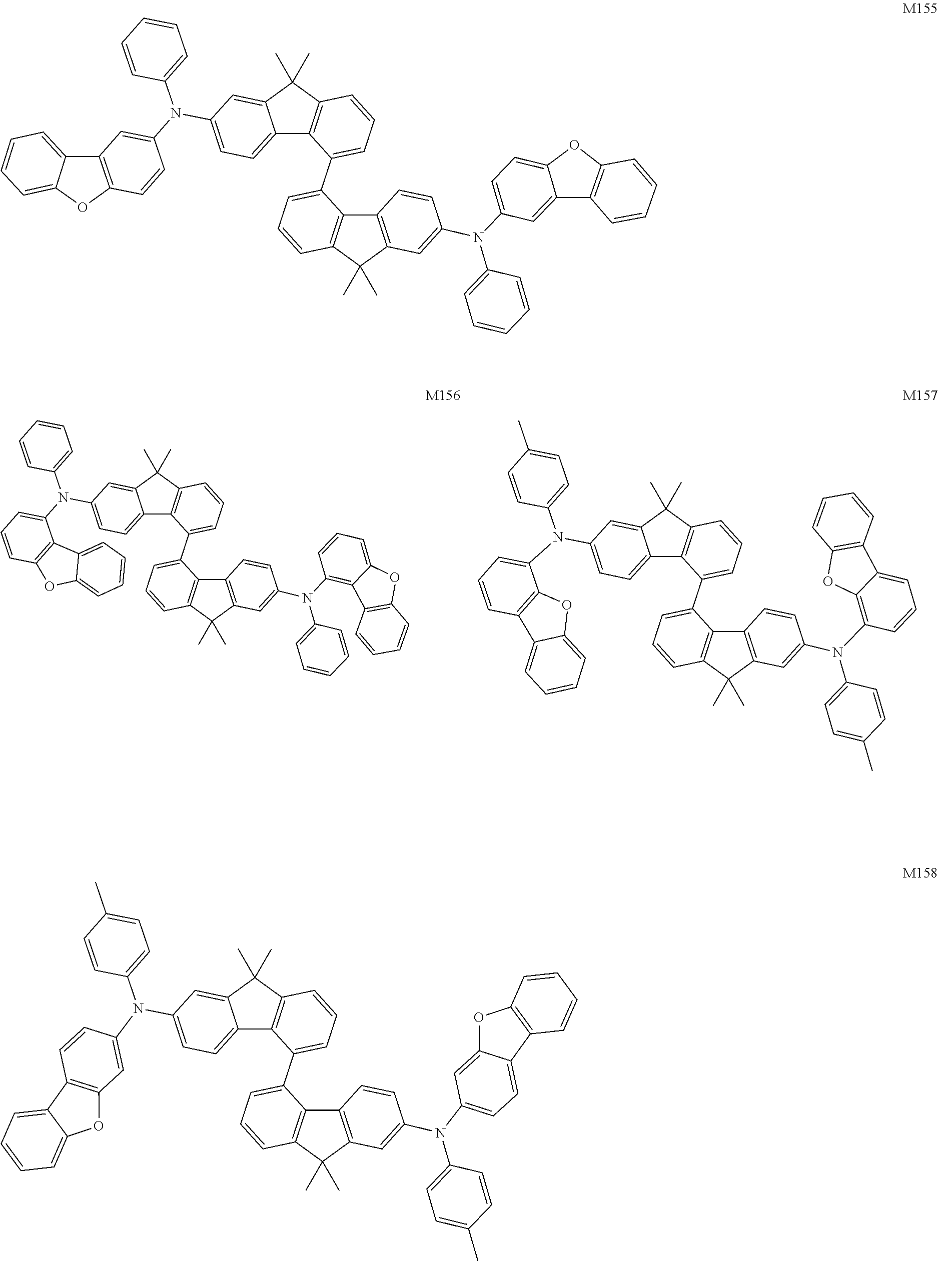 [SMILES: CC1(C)c2cc(N(c3ccccc3)c3ccc4oc5ccccc5c4c3)ccc2-c2c(-c3cccc4c3-c3ccc(N(c5ccccc5)c5ccc6oc7ccccc7c6c5)cc3C4(C)C)cccc21 ; CC1(C)c2cc(N(c3ccccc3)c3cccc4oc5ccccc5c34)ccc2-c2c(-c3cccc4c3-c3ccc(N(c5ccccc5)c5cccc6oc7ccccc7c56)cc3C4(C)C)cccc21 ; Cc1ccc(N(c2ccc3c(c2)C(C)(C)c2cccc(-c4cccc5c4-c4ccc(N(c6ccc(C)cc6)c6cccc7c6oc6ccccc67)cc4C5(C)C)c2-3)c2cccc3c2oc2ccccc23)cc1 ; Cc1ccc(N(c2ccc3c(c2)C(C)(C)c2cccc(-c4cccc5c4-c4ccc(N(c6ccc(C)cc6)c6ccc7c(c6)oc6ccccc67)cc4C5(C)C)c2-3)c2ccc3c(c2)oc2ccccc23)cc1]

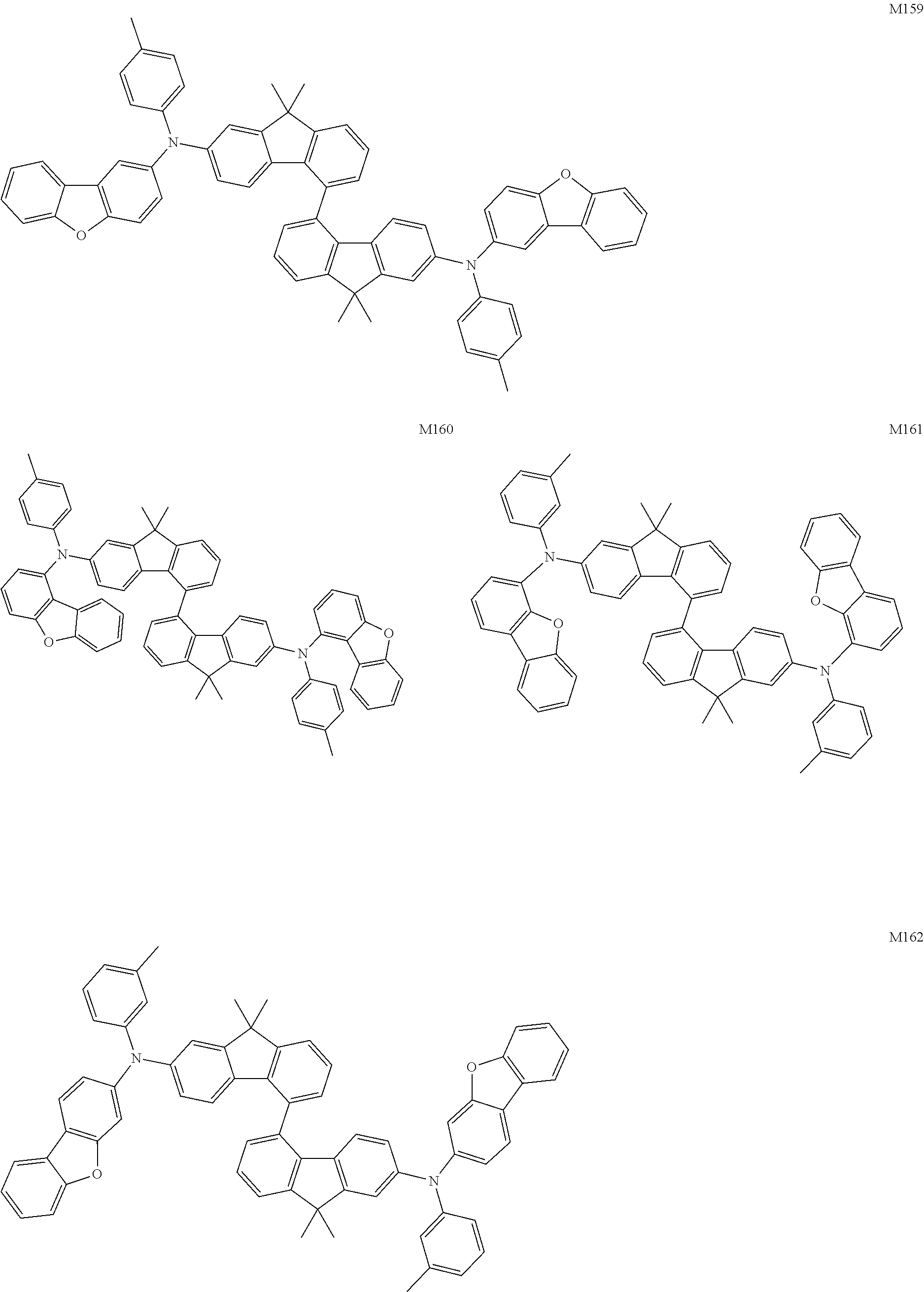

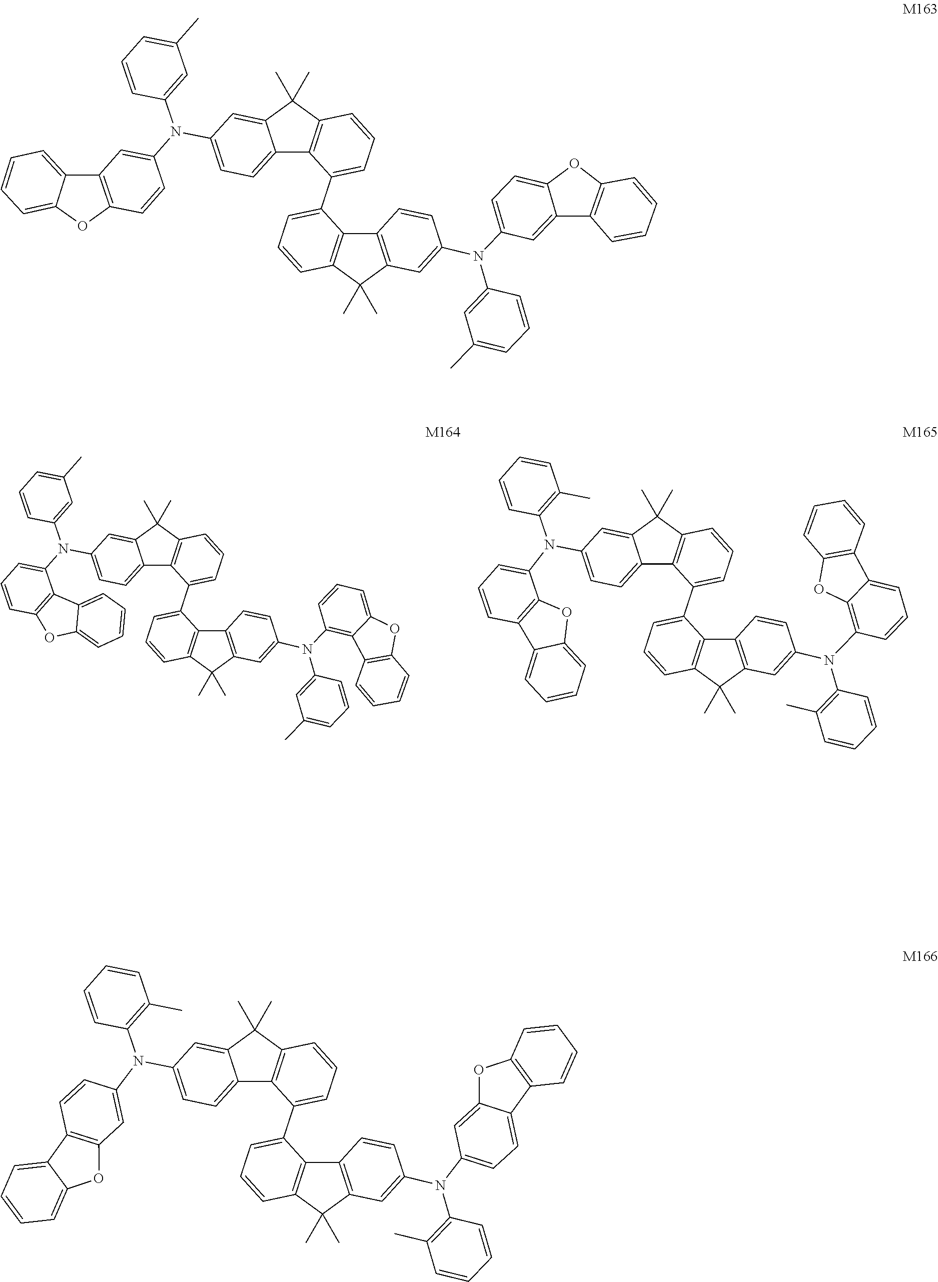 [SMILES: Cc1cccc(N(c2ccc3c(c2)C(C)(C)c2cccc(-c4cccc5c4-c4ccc(N(c6cccc(C)c6)c6ccc7oc8ccccc8c7c6)cc4C5(C)C)c2-3)c2ccc3oc4ccccc4c3c2)c1 ; Cc1cccc(N(c2ccc3c(c2)C(C)(C)c2cccc(-c4cccc5c4-c4ccc(N(c6cccc(C)c6)c6cccc7oc8ccccc8c67)cc4C5(C)C)c2-3)c2cccc3oc4ccccc4c23)c1 ; Cc1ccccc1N(c1ccc2c(c1)C(C)(C)c1cccc(-c3cccc4c3-c3ccc(N(c5ccccc5C)c5cccc6c5oc5ccccc56)cc3C4(C)C)c1-2)c1cccc2c1oc1ccccc12 ; Cc1ccccc1N(c1ccc2c(c1)C(C)(C)c1cccc(-c3cccc4c3-c3ccc(N(c5ccc6c(c5)oc5ccccc56)c5ccccc5C)cc3C4(C)C)c1-2)c1ccc2c(c1)oc1ccccc12]

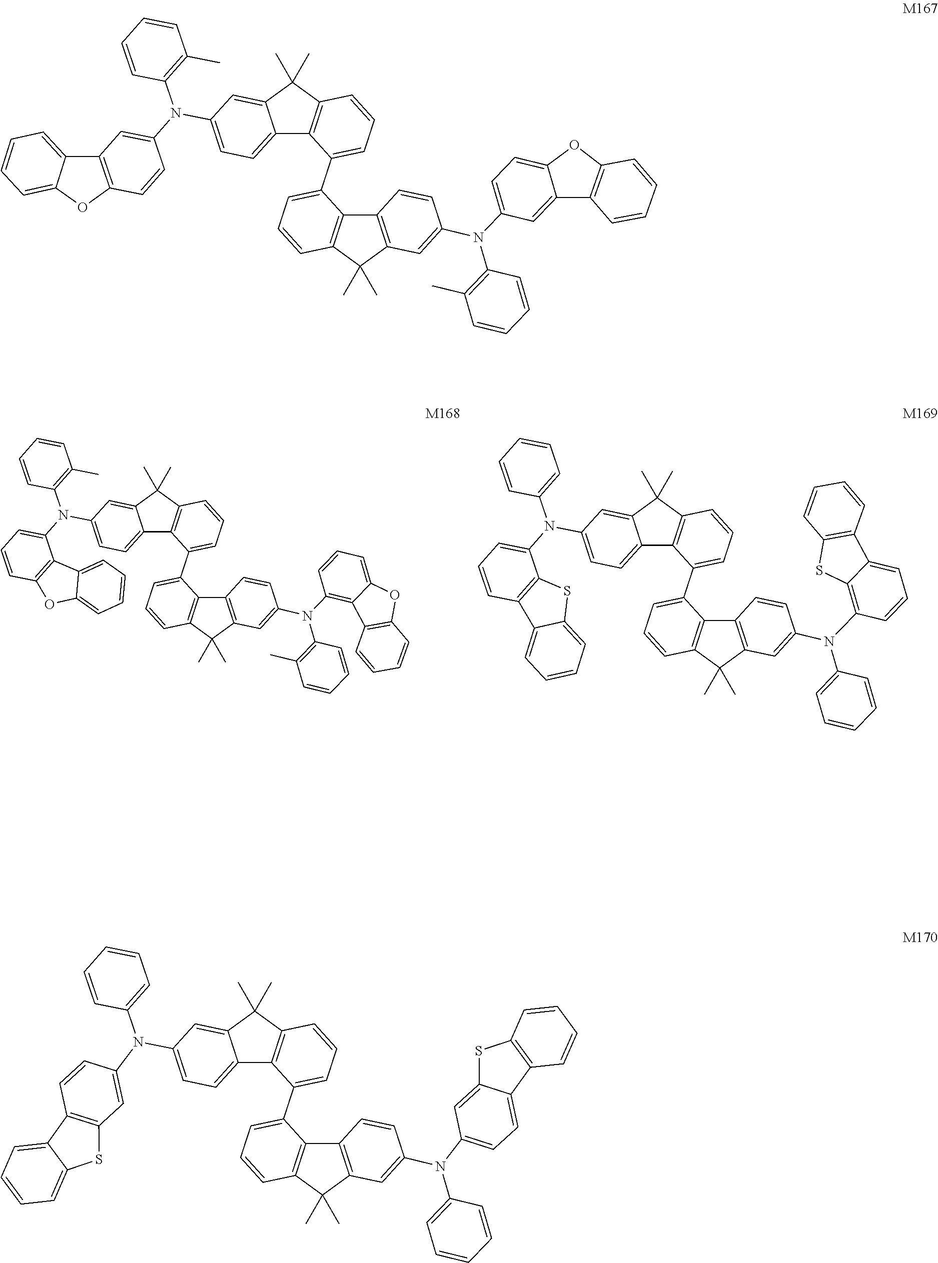 [SMILES: Cc1ccccc1N(c1ccc2c(c1)C(C)(C)c1cccc(-c3cccc4c3-c3ccc(N(c5ccc6oc7ccccc7c6c5)c5ccccc5C)cc3C4(C)C)c1-2)c1ccc2oc3ccccc3c2c1 ; Cc1ccccc1N(c1ccc2c(c1)C(C)(C)c1cccc(-c3cccc4c3-c3ccc(N(c5ccccc5C)c5cccc6oc7ccccc7c56)cc3C4(C)C)c1-2)c1cccc2oc3ccccc3c12 ; CC1(C)c2cc(N(c3ccccc3)c3cccc4c3sc3ccccc34)ccc2-c2c(-c3cccc4c3-c3ccc(N(c5ccccc5)c5cccc6c5sc5ccccc56)cc3C4(C)C)cccc21 ; CC1(C)c2cc(N(c3ccccc3)c3ccc4c(c3)sc3ccccc34)ccc2-c2c(-c3cccc4c3-c3ccc(N(c5ccccc5)c5ccc6c(c5)sc5ccccc56)cc3C4(C)C)cccc21]

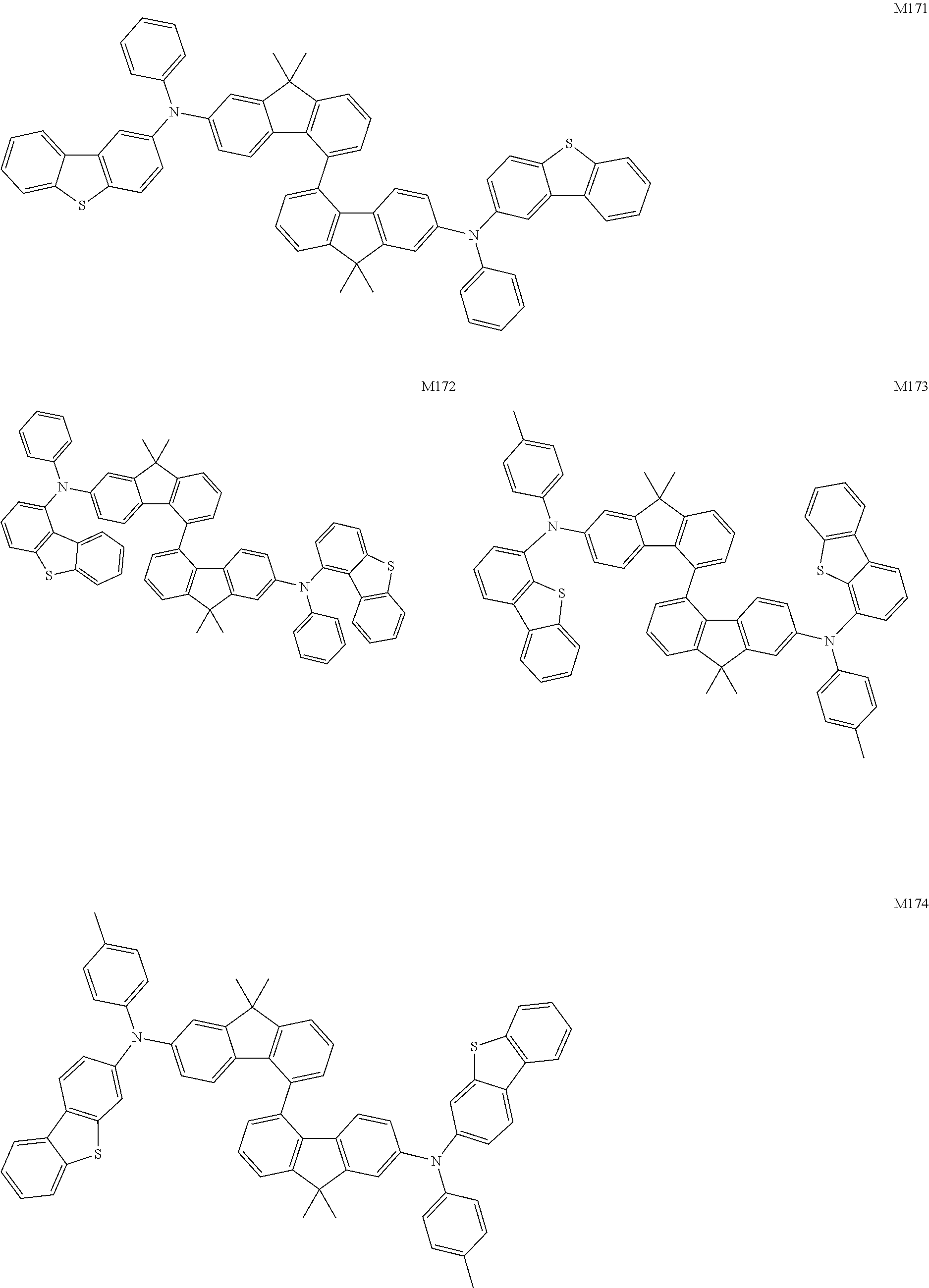 [SMILES: CC1(C)c2cc(N(c3ccccc3)c3ccc4sc5ccccc5c4c3)ccc2-c2c(-c3cccc4c3-c3ccc(N(c5ccccc5)c5ccc6sc7ccccc7c6c5)cc3C4(C)C)cccc21 ; CC1(C)c2cc(N(c3ccccc3)c3cccc4sc5ccccc5c34)ccc2-c2c(-c3cccc4c3-c3ccc(N(c5ccccc5)c5cccc6sc7ccccc7c56)cc3C4(C)C)cccc21 ; Cc1ccc(N(c2ccc3c(c2)C(C)(C)c2cccc(-c4cccc5c4-c4ccc(N(c6ccc(C)cc6)c6cccc7c6sc6ccccc67)cc4C5(C)C)c2-3)c2cccc3c2sc2ccccc23)cc1 ; Cc1ccc(N(c2ccc3c(c2)C(C)(C)c2cccc(-c4cccc5c4-c4ccc(N(c6ccc(C)cc6)c6ccc7c(c6)sc6ccccc67)cc4C5(C)C)c2-3)c2ccc3c(c2)sc2ccccc23)cc1]

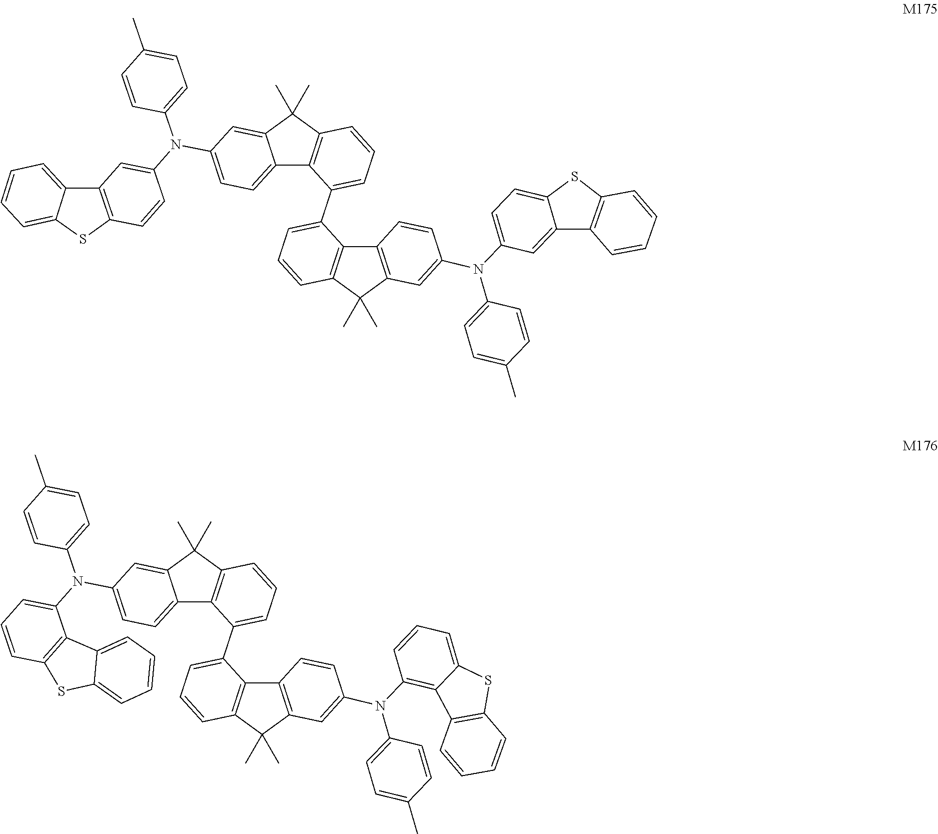 [SMILES: Cc1ccc(N(c2ccc3c(c2)C(C)(C)c2cccc(-c4cccc5c4-c4ccc(N(c6ccc(C)cc6)c6ccc7sc8ccccc8c7c6)cc4C5(C)C)c2-3)c2ccc3sc4ccccc4c3c2)cc1 ; Cc1ccc(N(c2ccc3c(c2)C(C)(C)c2cccc(-c4cccc5c4-c4ccc(N(c6ccc(C)cc6)c6cccc7sc8ccccc8c67)cc4C5(C)C)c2-3)c2cccc3sc4ccccc4c23)cc1]

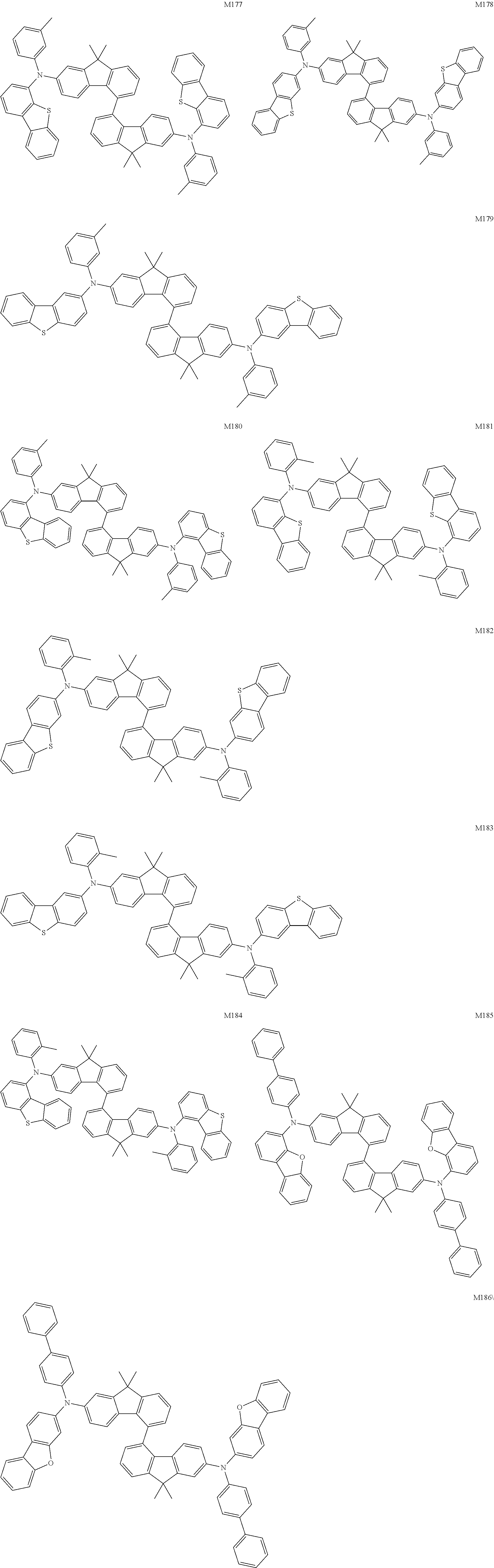

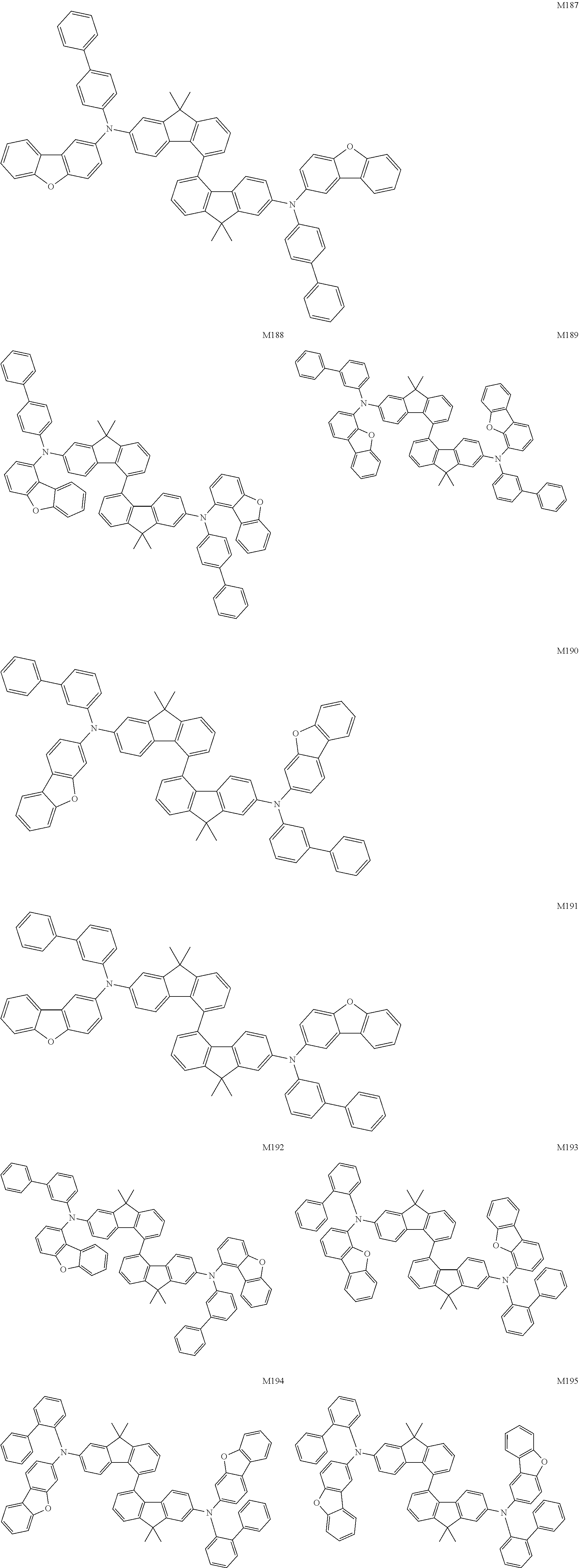

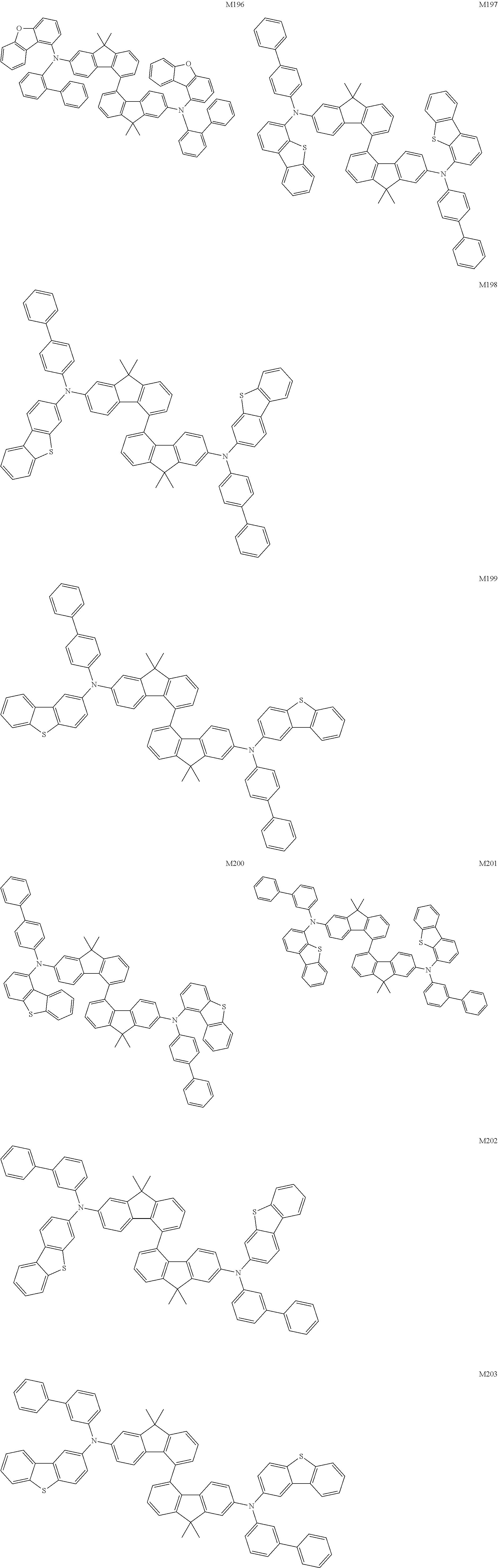

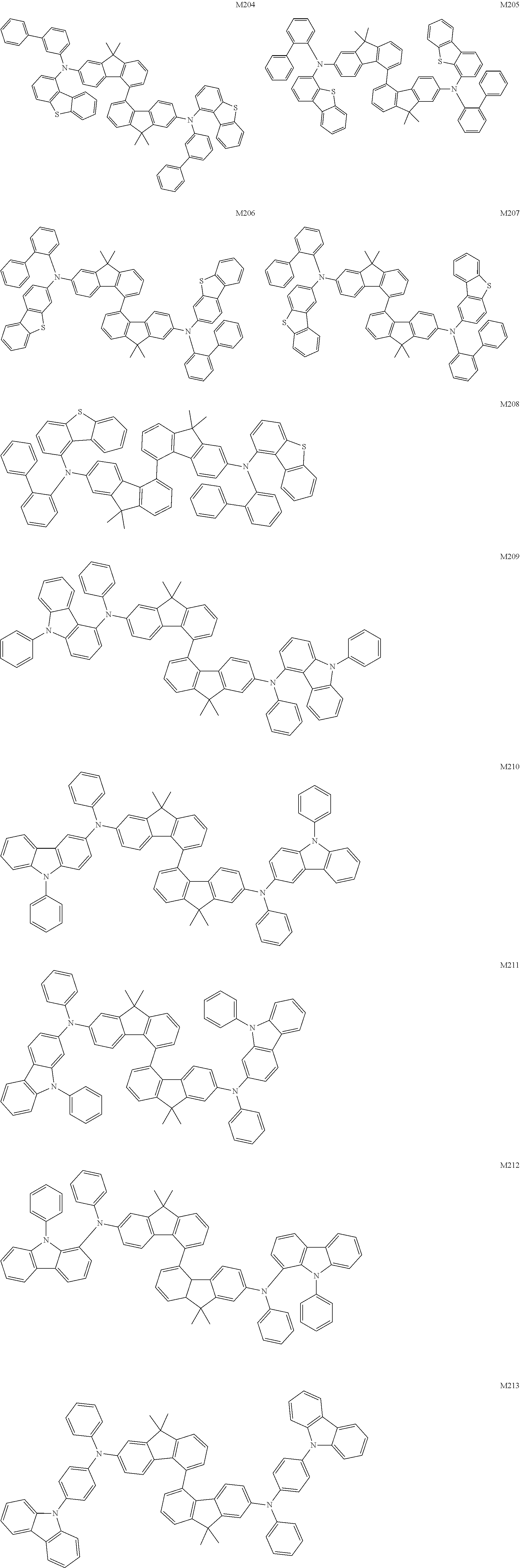

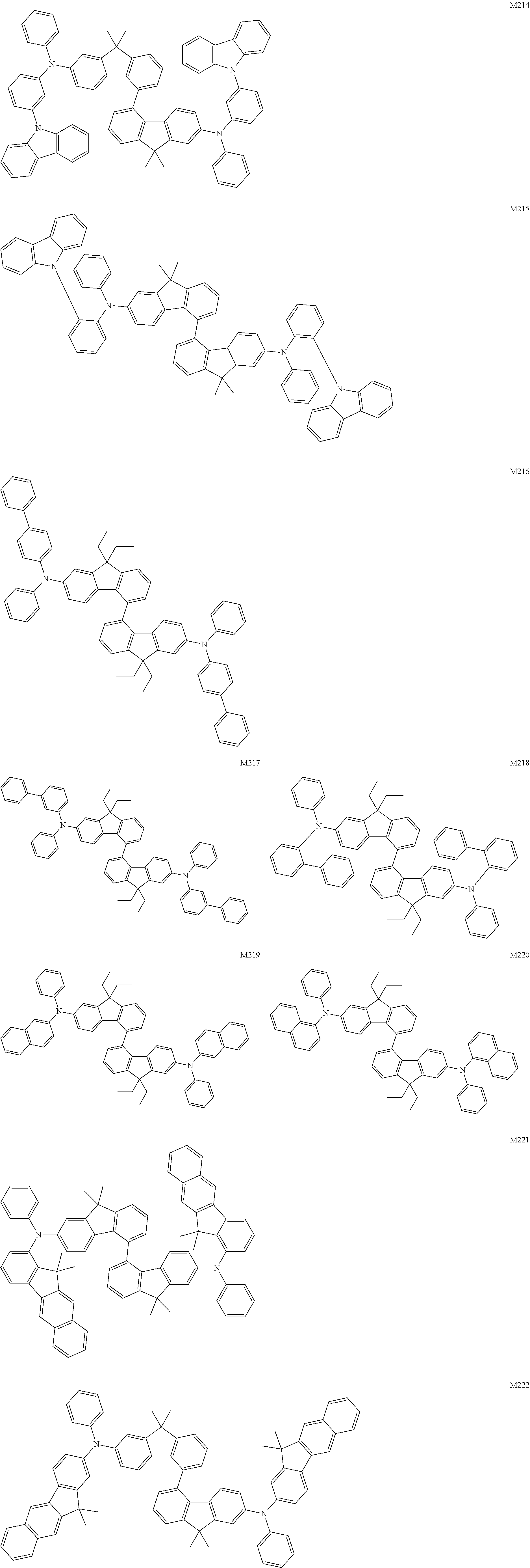

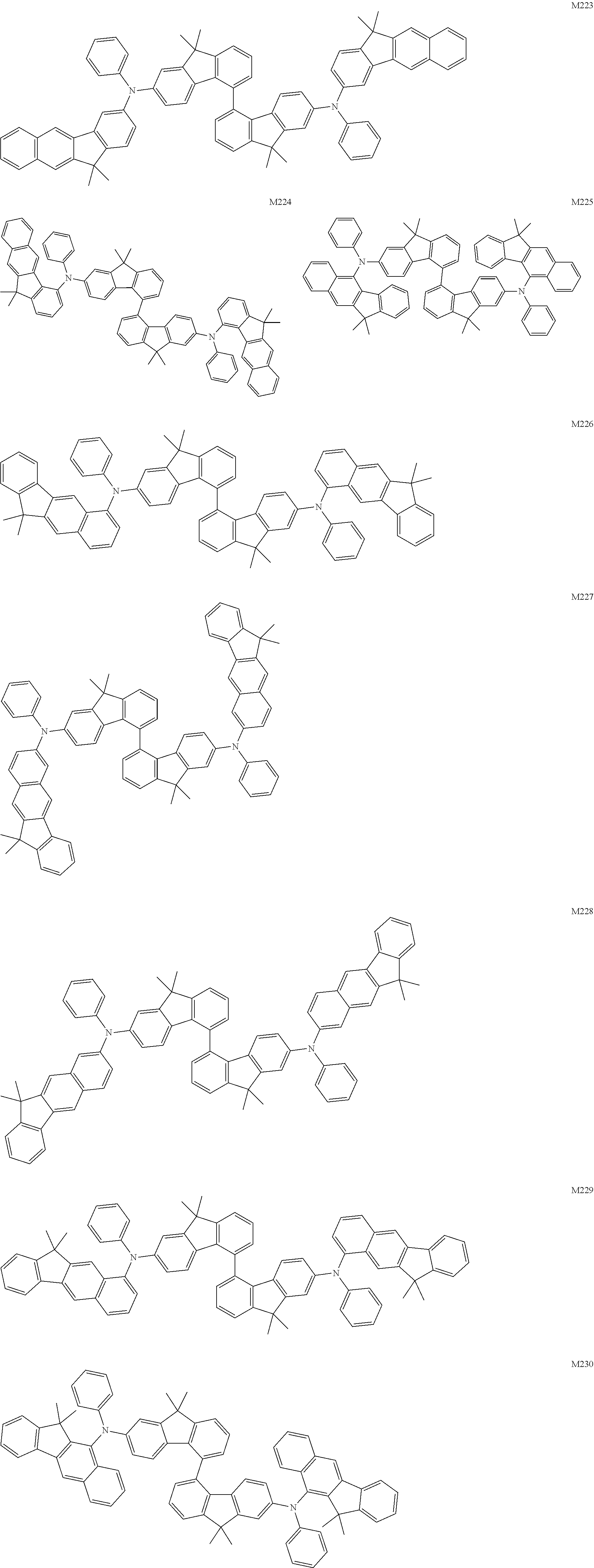

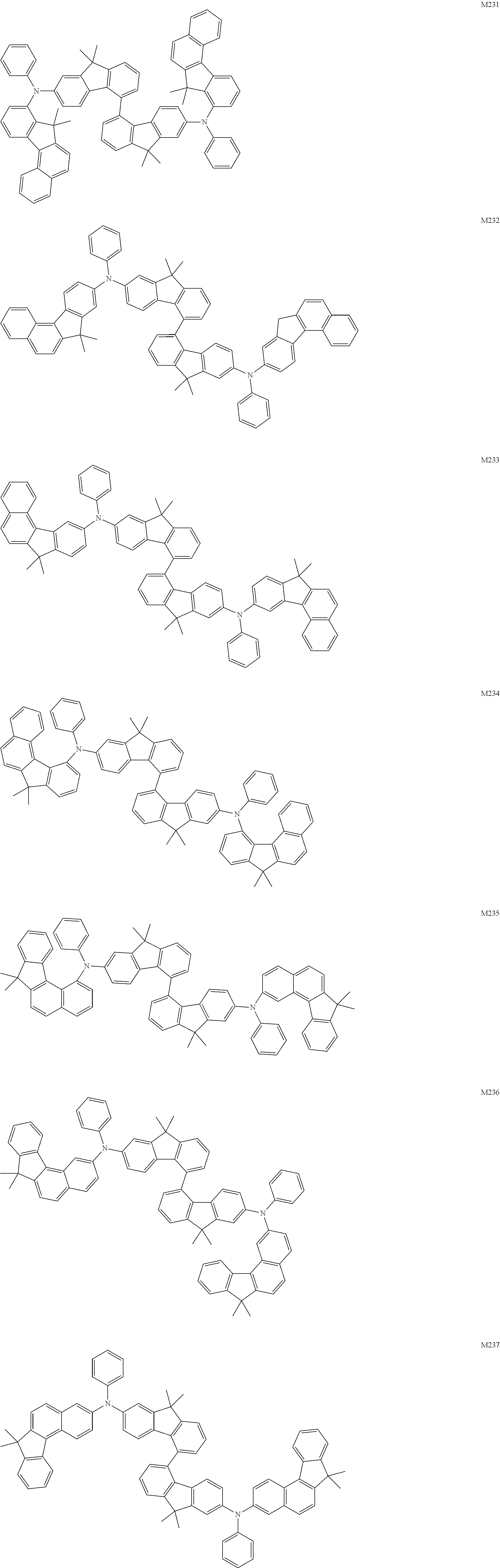

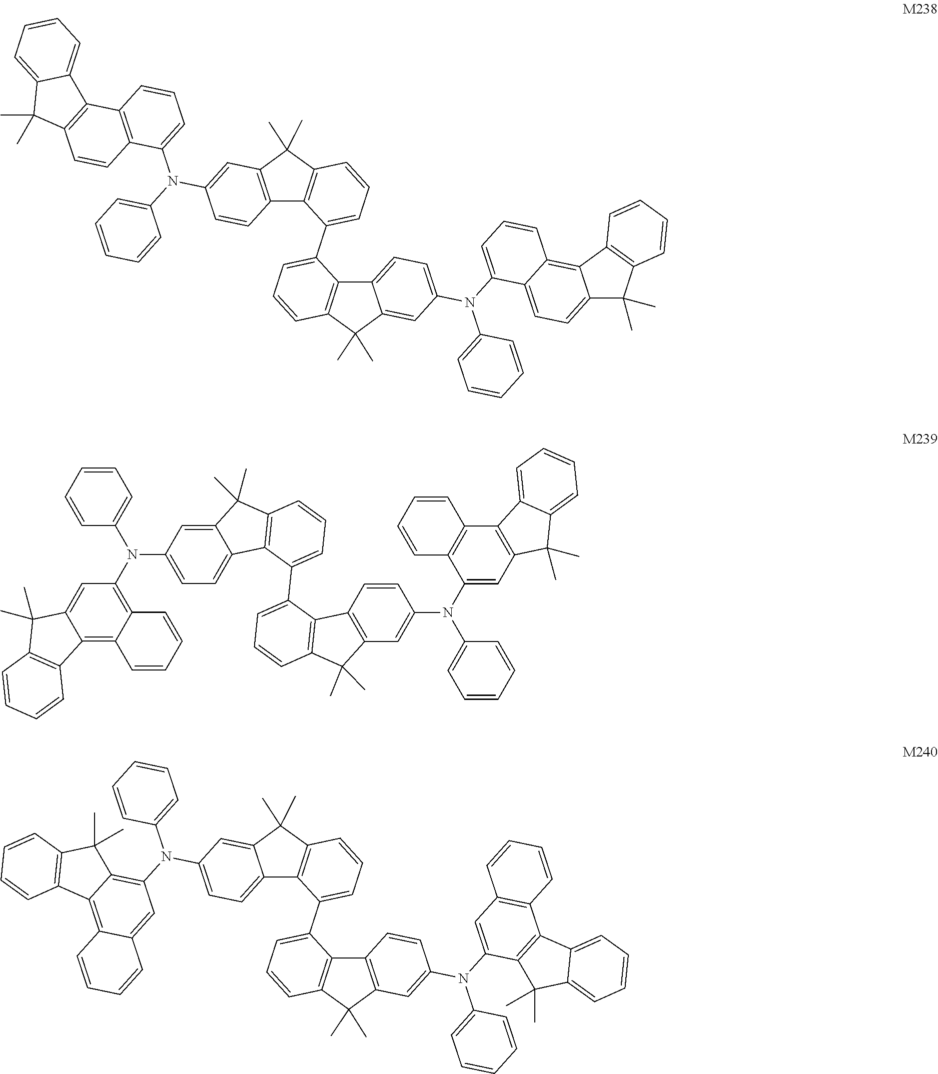

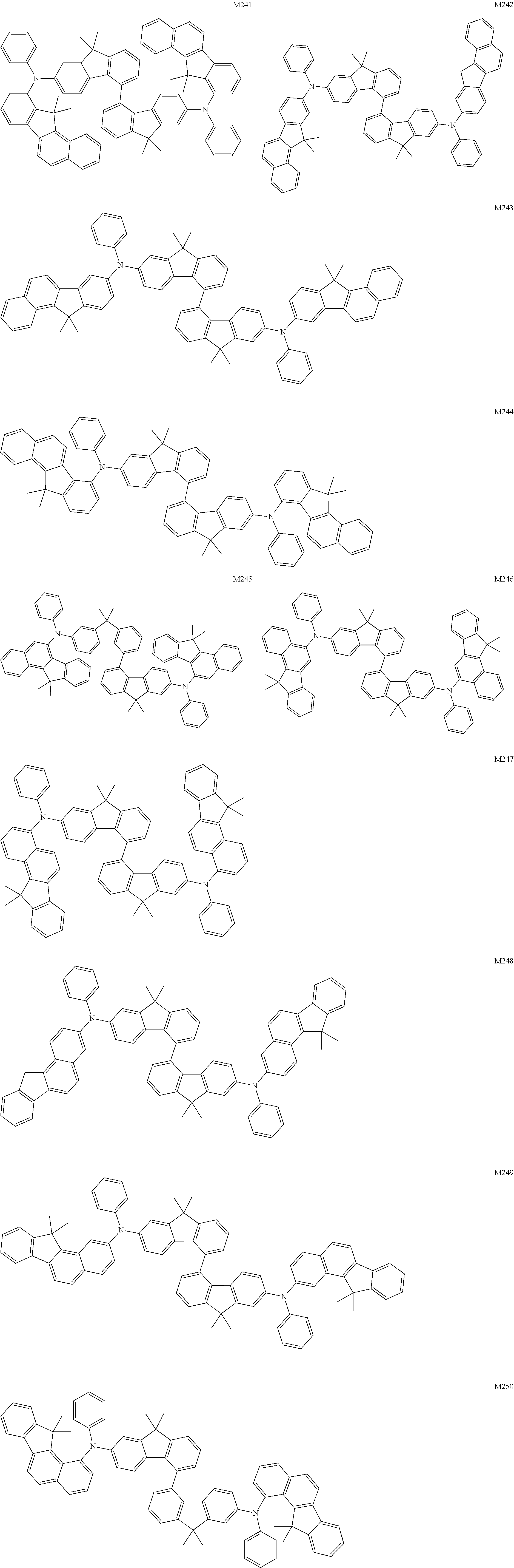

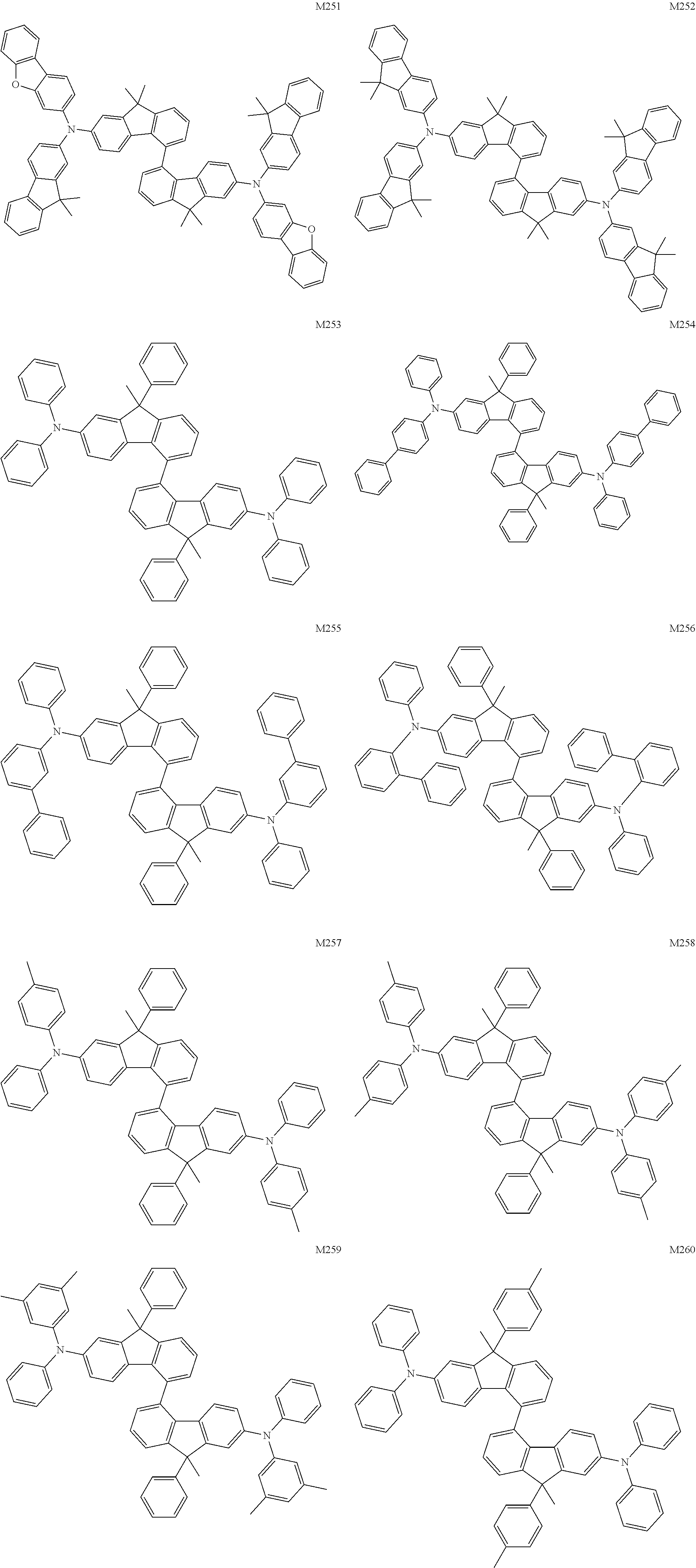

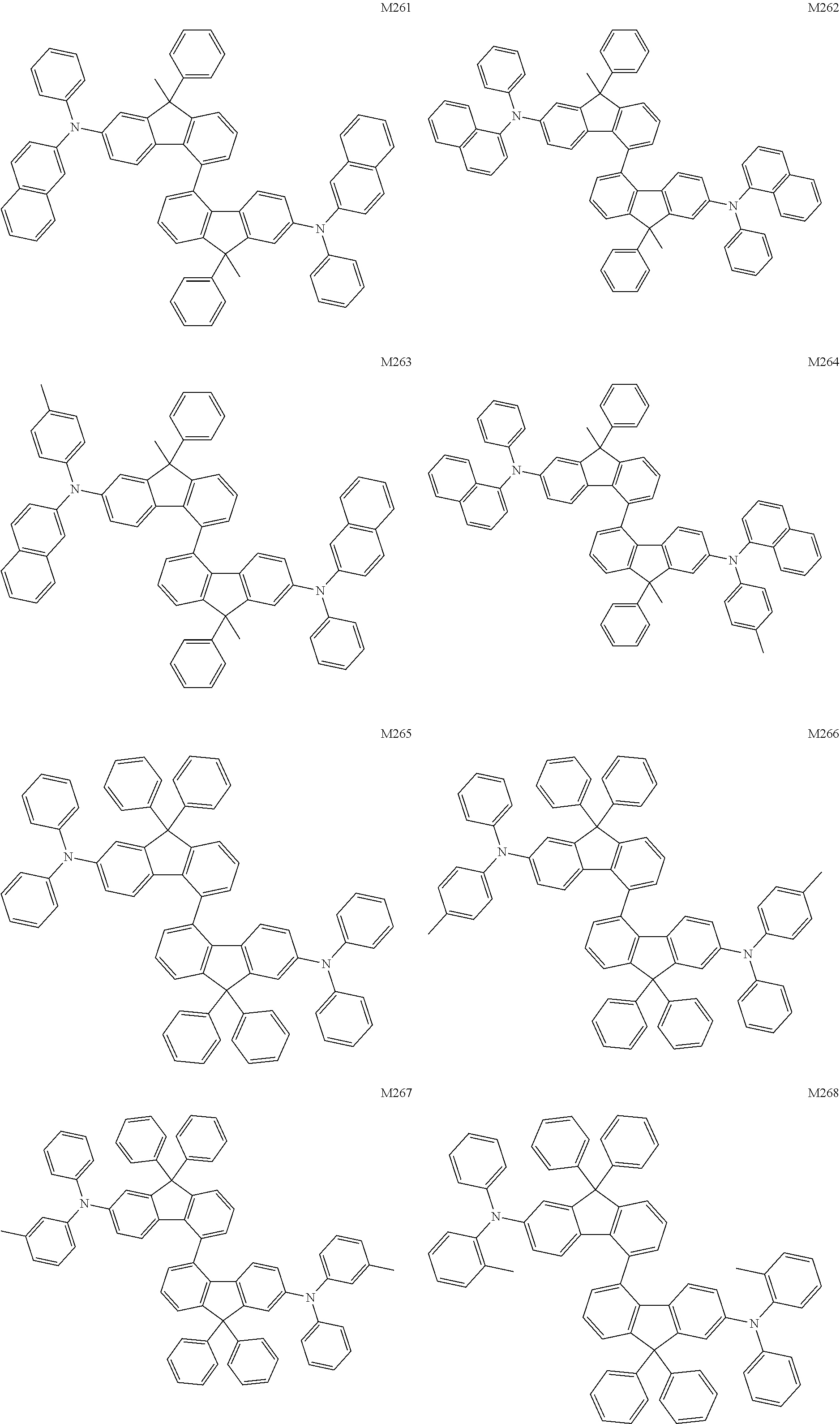

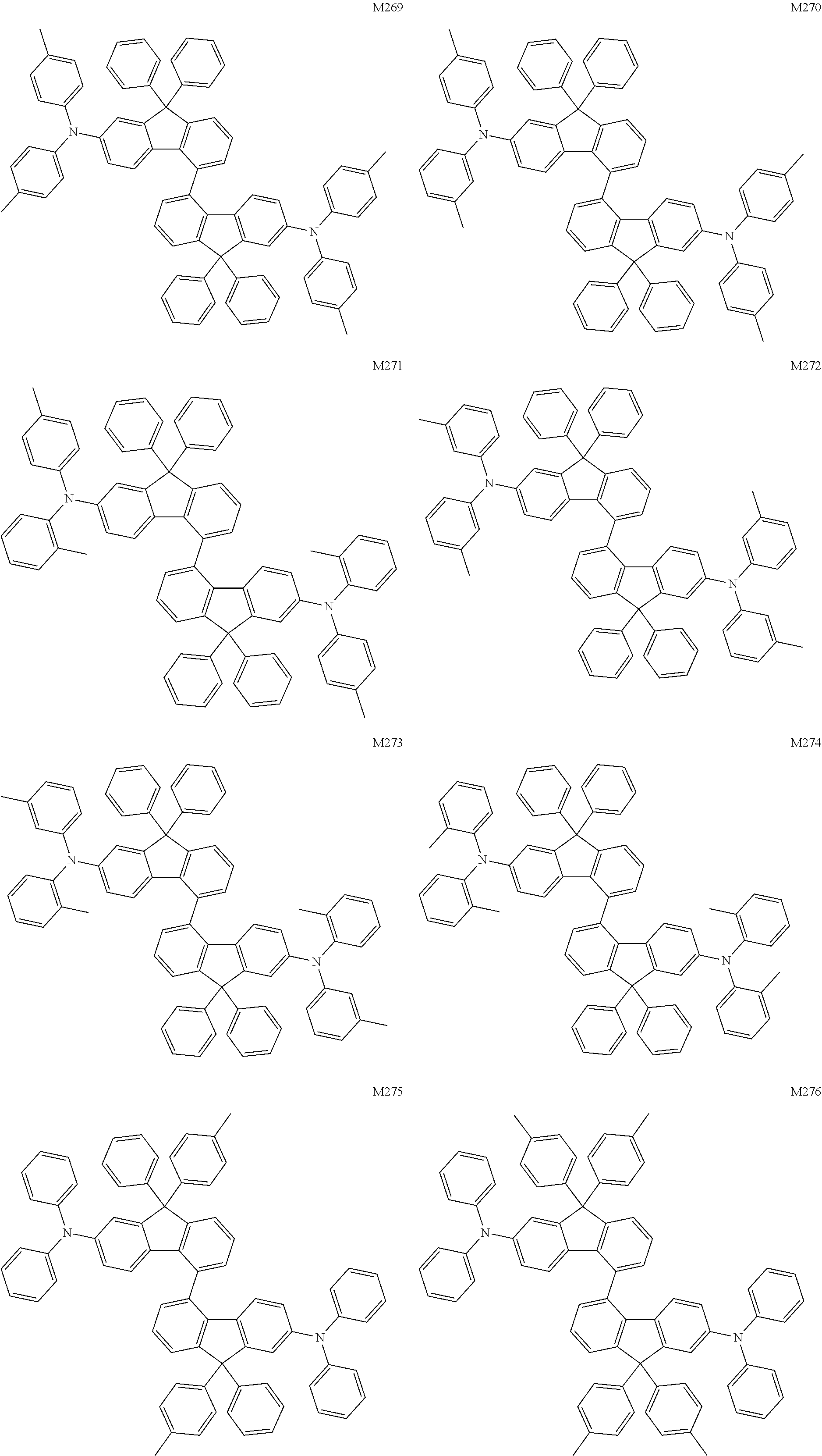

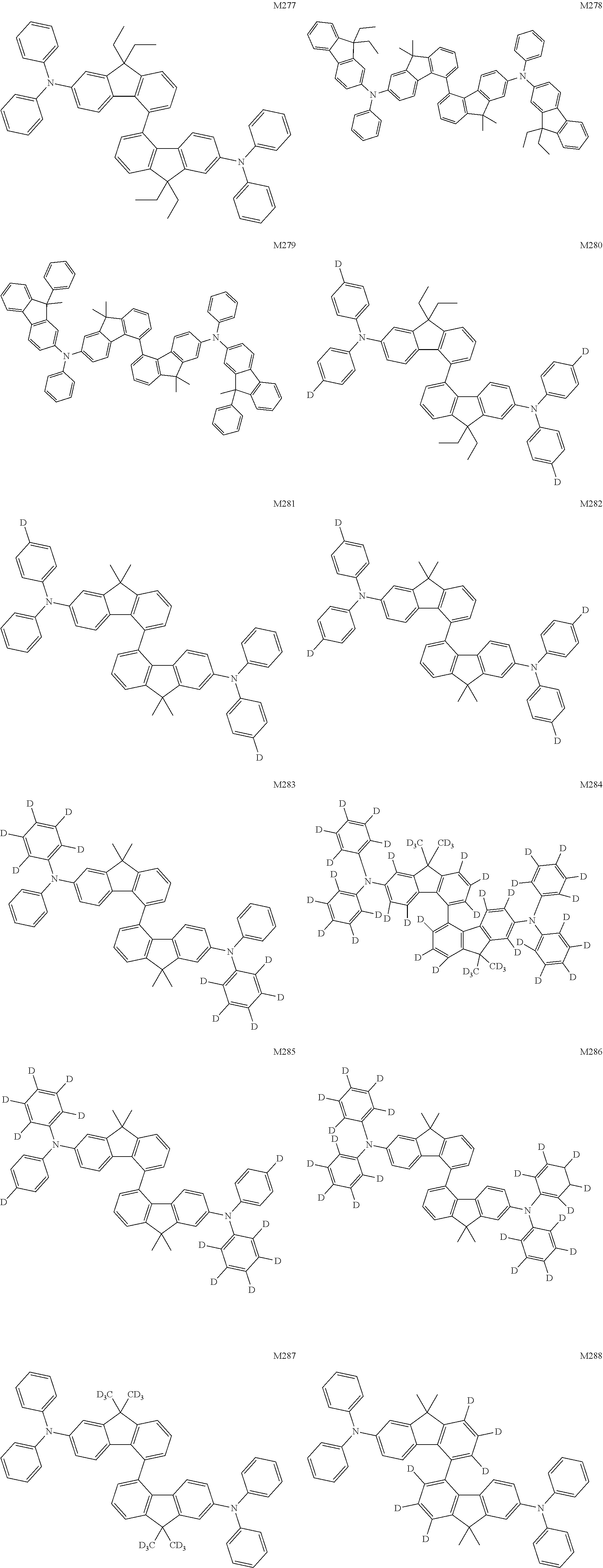

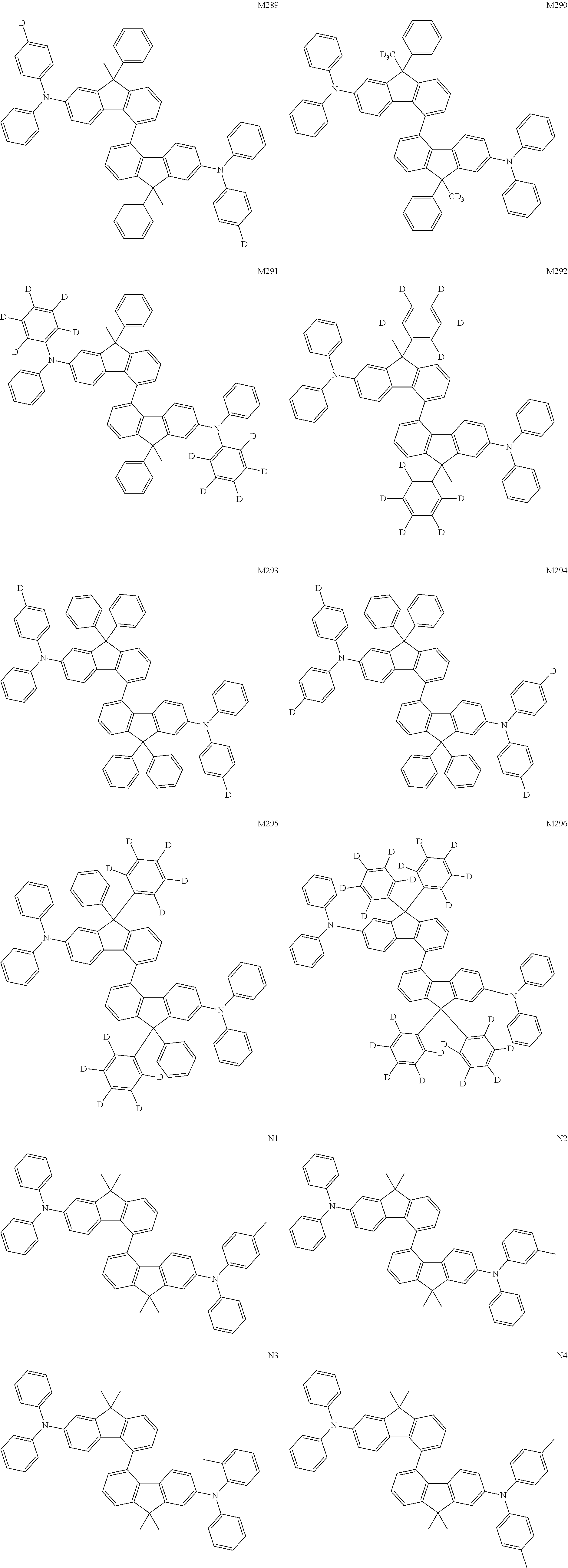

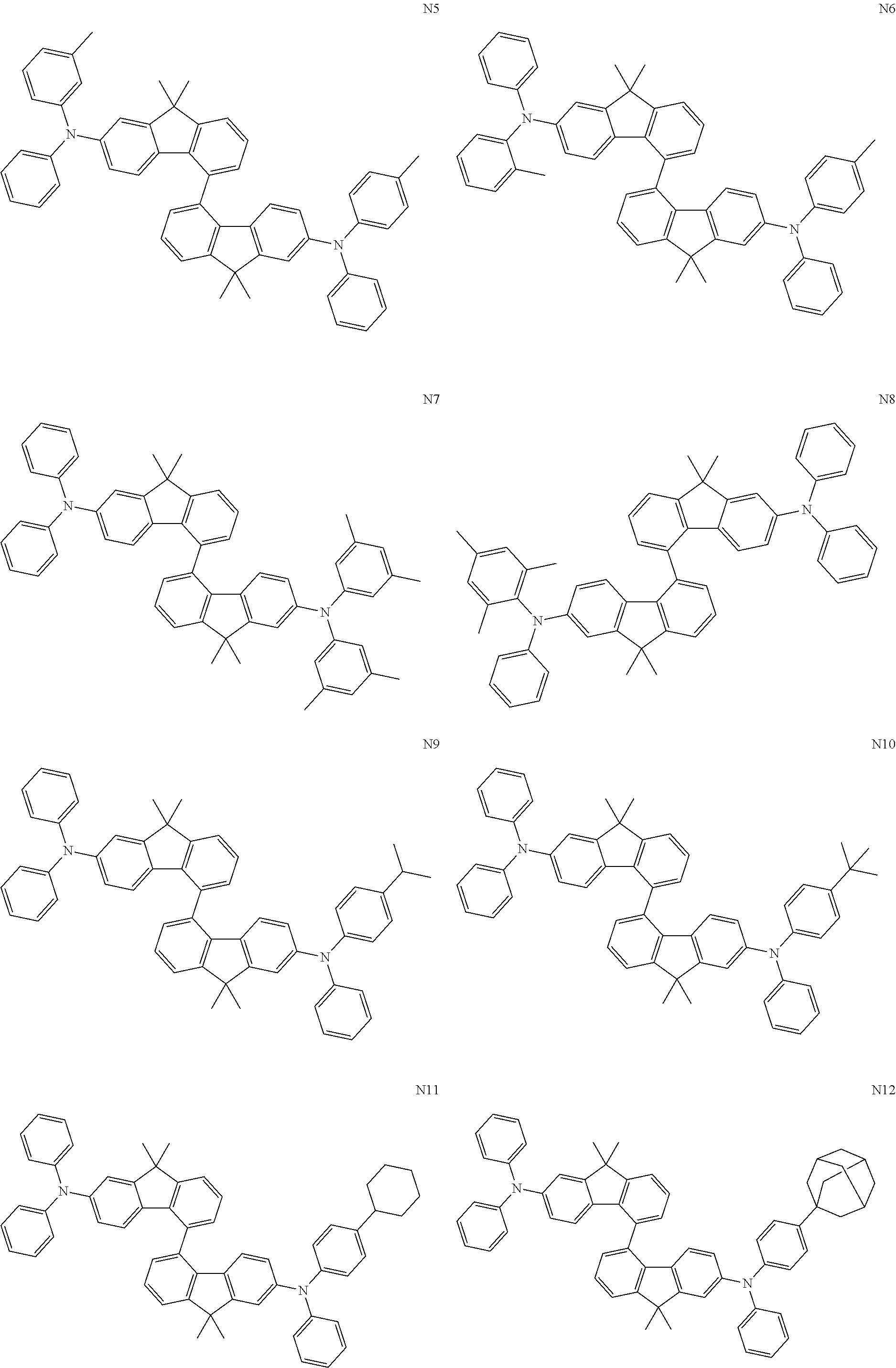

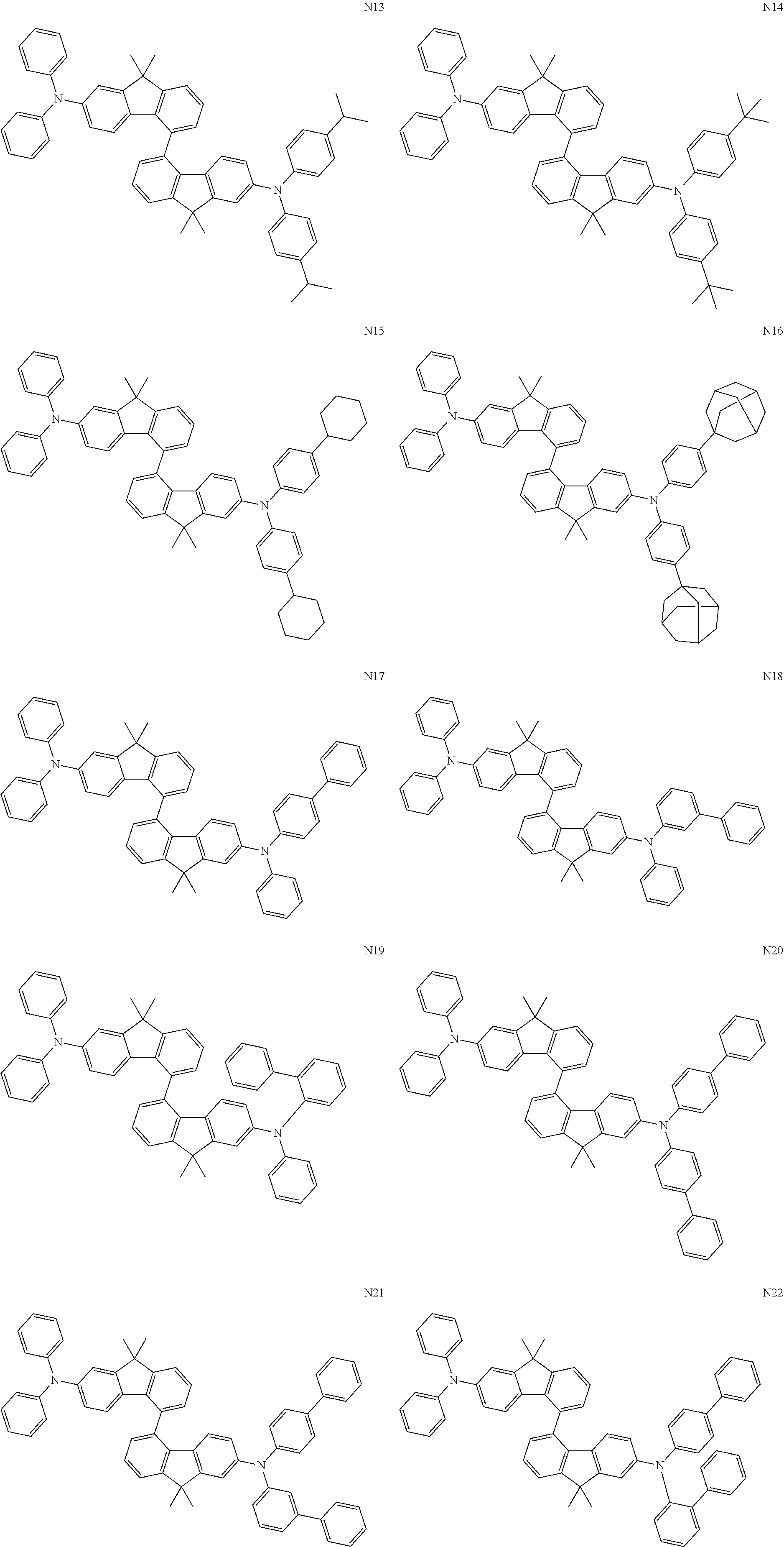

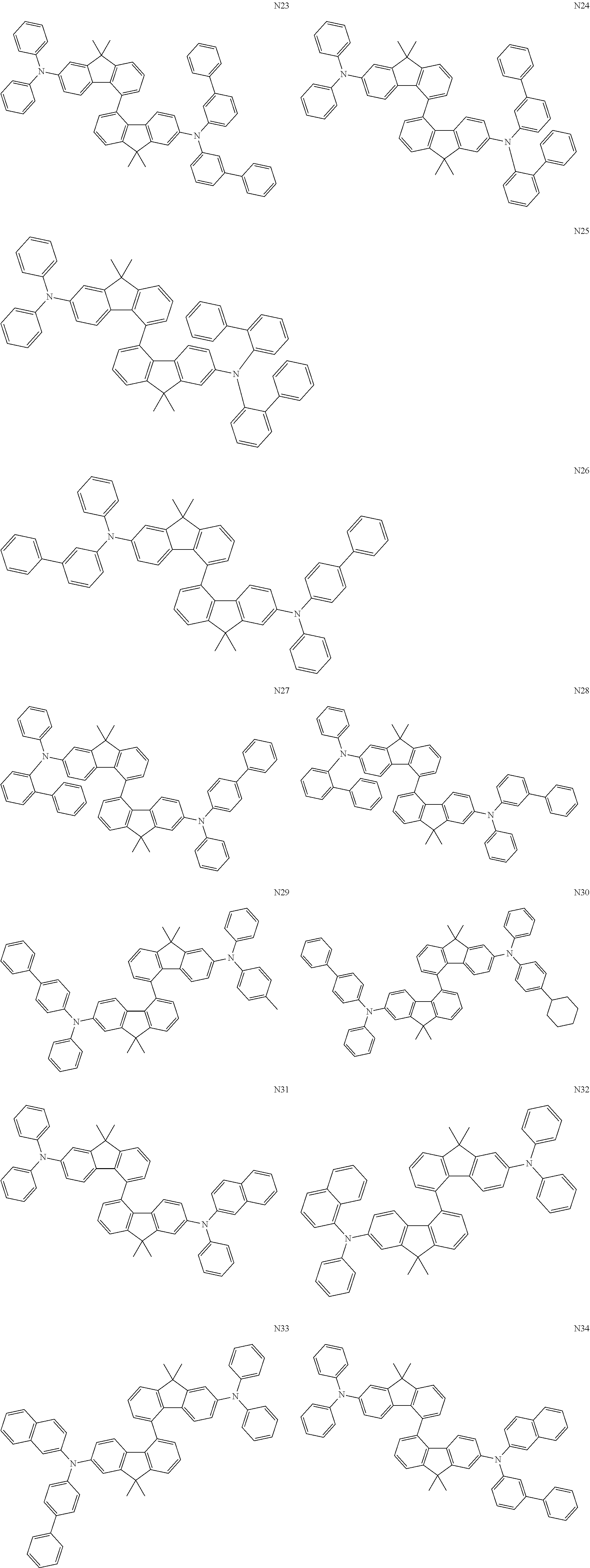

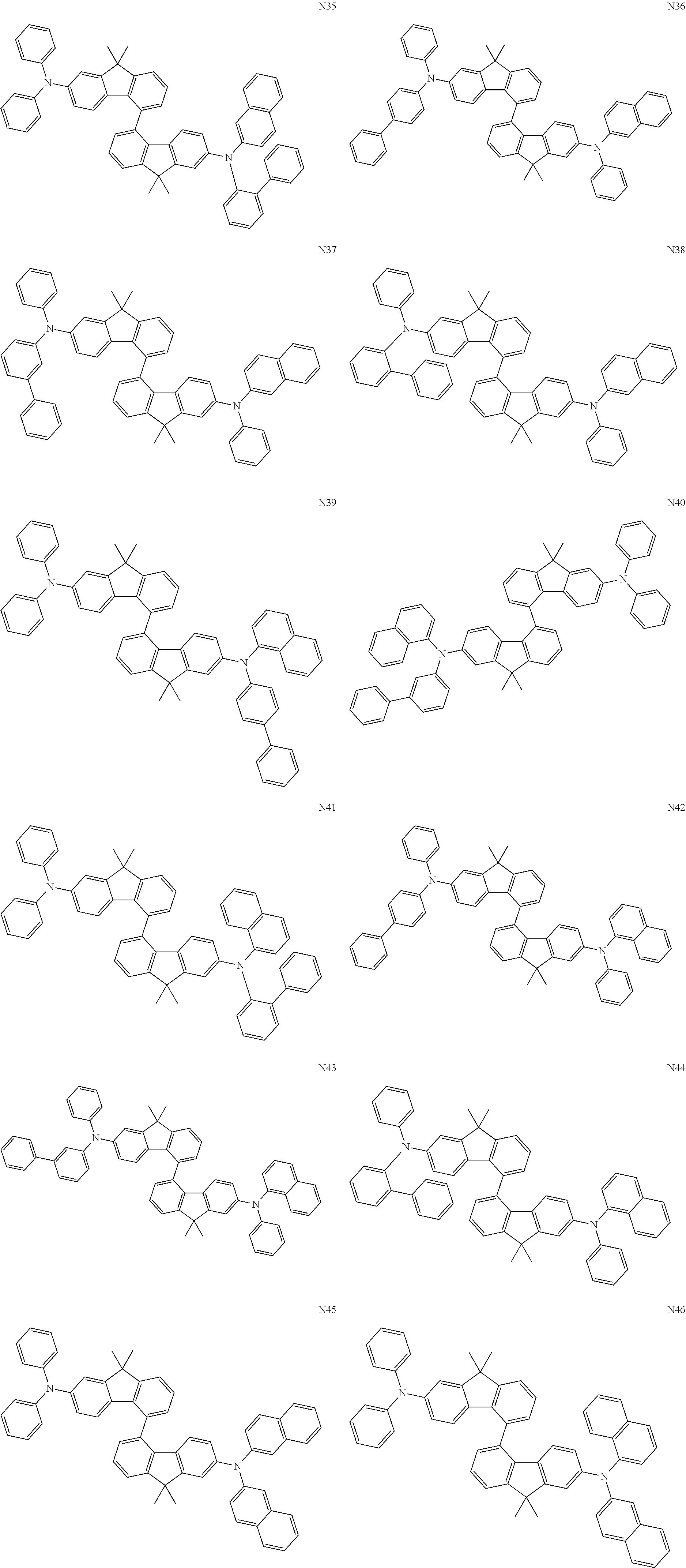

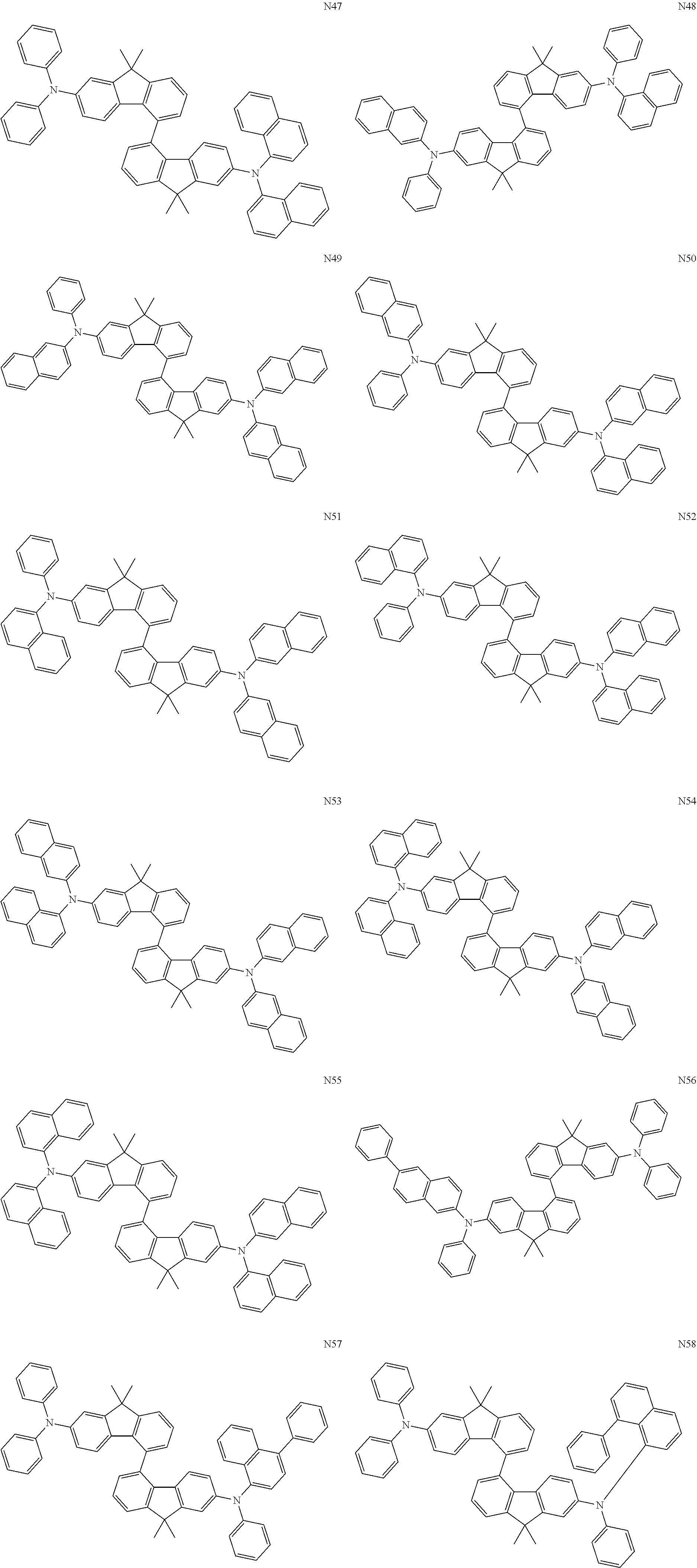

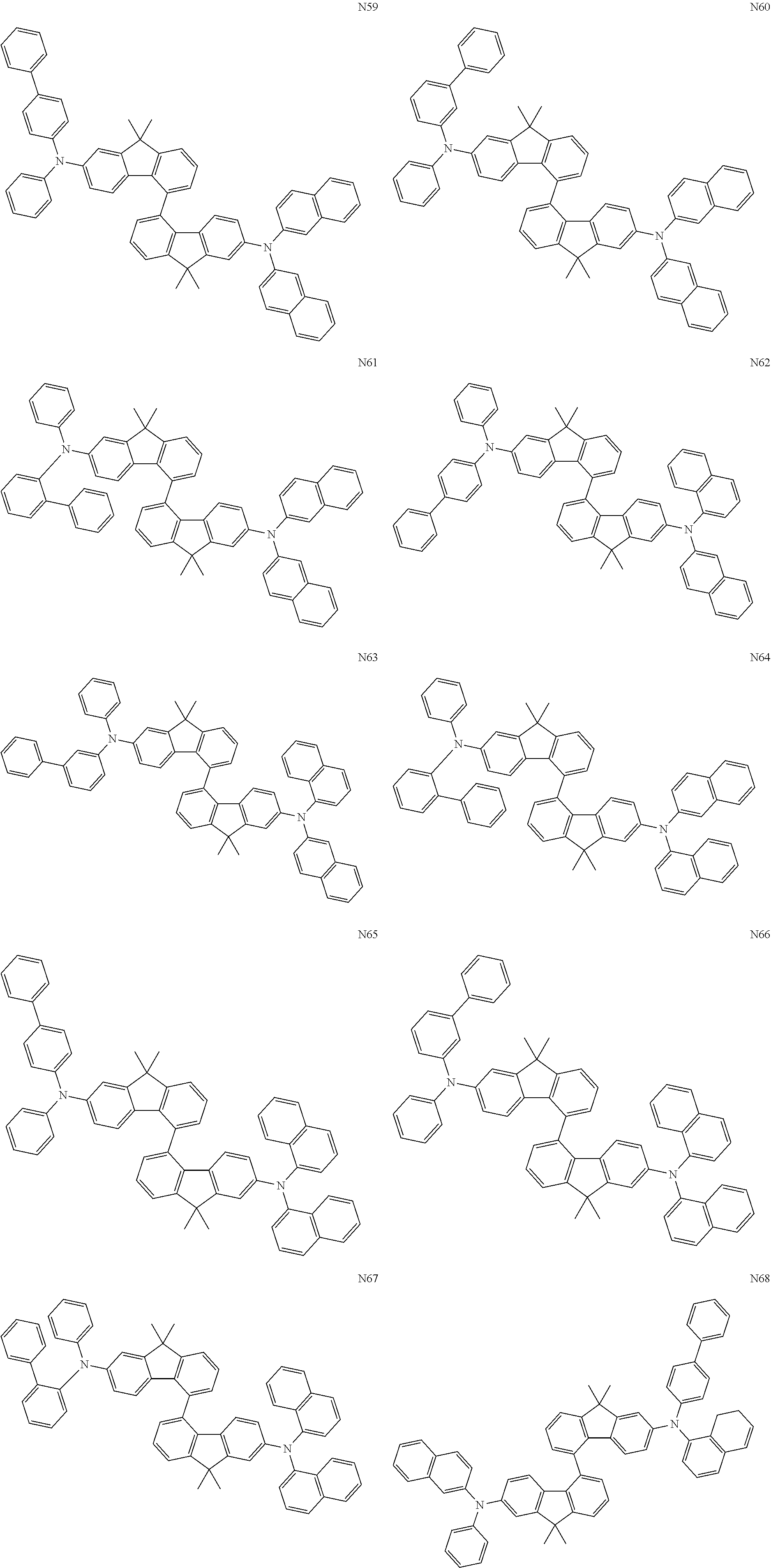

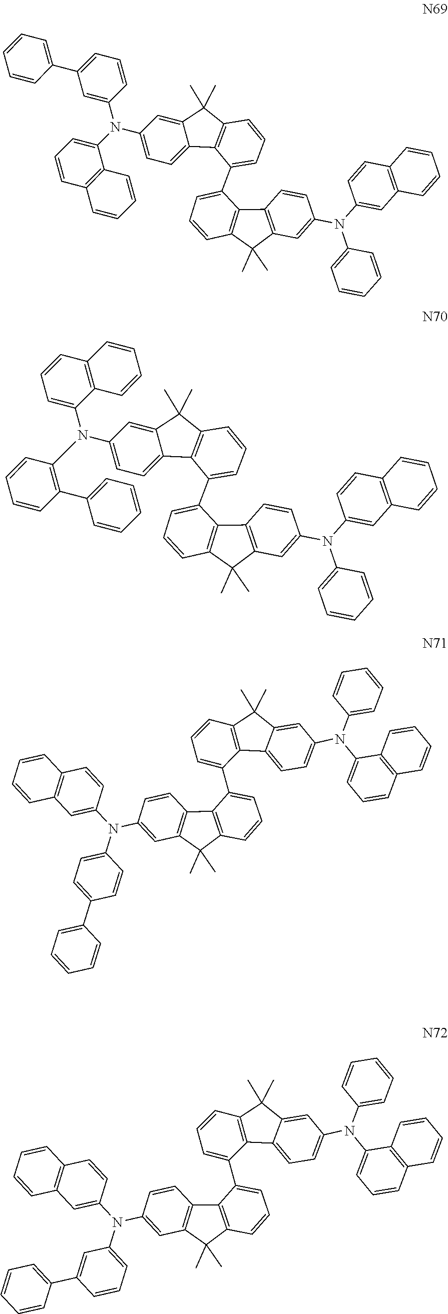 [SMILES: CC1(C)c2cc(N(c3ccccc3)c3ccc4ccccc4c3)ccc2-c2c(-c3cccc4c3-c3ccc(N(c5cccc(-c6ccccc6)c5)c5cccc6ccccc56)cc3C4(C)C)cccc21 ; CC1(C)c2cc(N(c3ccccc3)c3ccc4ccccc4c3)ccc2-c2c(-c3cccc4c3-c3ccc(N(c5ccccc5-c5ccccc5)c5cccc6ccccc56)cc3C4(C)C)cccc21 ; CC1(C)c2cc(N(c3ccc(-c4ccccc4)cc3)c3ccc4ccccc4c3)ccc2-c2c(-c3cccc4c3-c3ccc(N(c5ccccc5)c5cccc6ccccc56)cc3C4(C)C)cccc21 ; CC1(C)c2cc(N(c3cccc(-c4ccccc4)c3)c3ccc4ccccc4c3)ccc2-c2c(-c3cccc4c3-c3ccc(N(c5ccccc5)c5cccc6ccccc56)cc3C4(C)C)cccc21]

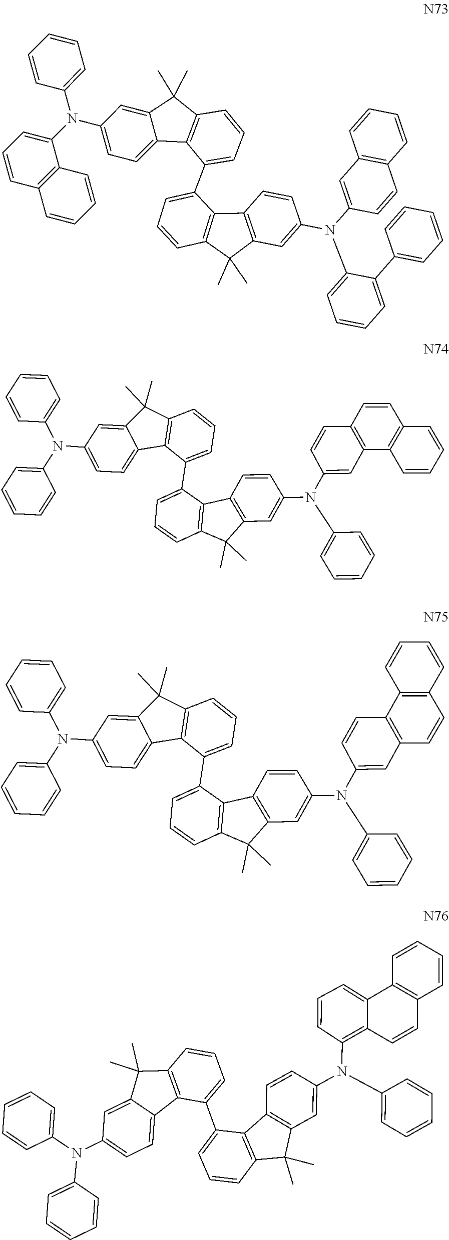 [SMILES: CC1(C)c2cc(N(c3ccc4ccccc4c3)c3ccccc3-c3ccccc3)ccc2-c2c(-c3cccc4c3-c3ccc(N(c5ccccc5)c5cccc6ccccc56)cc3C4(C)C)cccc21 ; CC1(C)c2cc(N(c3ccccc3)c3ccccc3)ccc2-c2c(-c3cccc4c3-c3ccc(N(c5ccccc5)c5ccc6ccc7ccccc7c6c5)cc3C4(C)C)cccc21 ; CC1(C)c2cc(N(c3ccccc3)c3ccccc3)ccc2-c2c(-c3cccc4c3-c3ccc(N(c5ccccc5)c5ccc6c(ccc7ccccc76)c5)cc3C4(C)C)cccc21 ; CC1(C)c2cc(N(c3ccccc3)c3ccccc3)ccc2-c2c(-c3cccc4c3-c3ccc(N(c5ccccc5)c5cccc6c5ccc5ccccc56)cc3C4(C)C)cccc21]

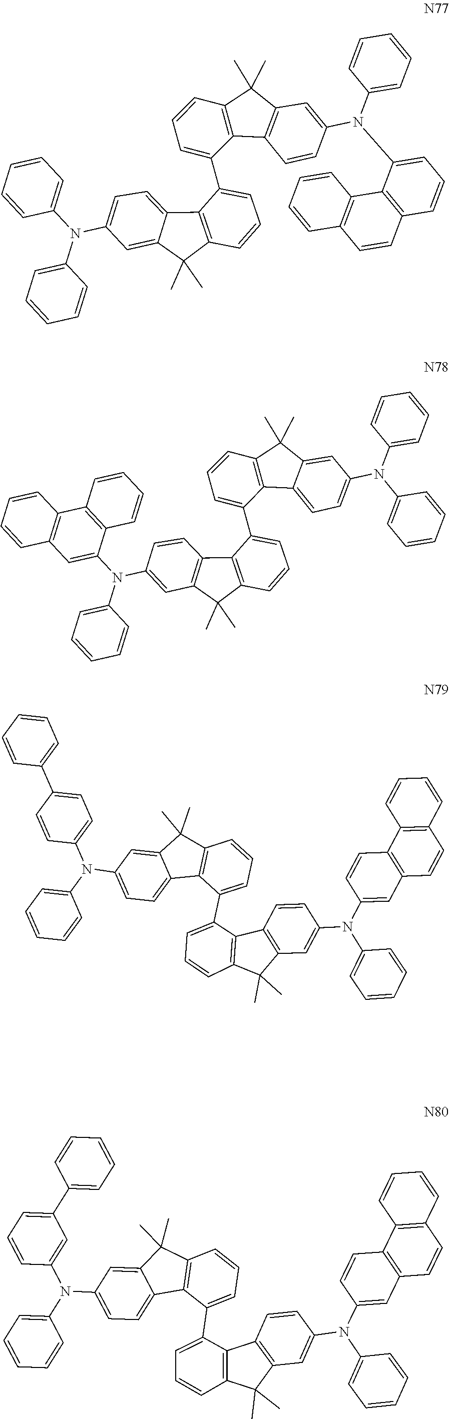 [SMILES: CC1(C)c2cc(N(c3ccccc3)c3ccccc3)ccc2-c2c(-c3cccc4c3-c3ccc(N(c5ccccc5)c5cccc6ccc7ccccc7c56)cc3C4(C)C)cccc21 ; CC1(C)c2cc(N(c3ccccc3)c3ccccc3)ccc2-c2c(-c3cccc4c3-c3ccc(N(c5ccccc5)c5cc6ccccc6c6ccccc56)cc3C4(C)C)cccc21 ; CC1(C)c2cc(N(c3ccccc3)c3ccc(-c4ccccc4)cc3)ccc2-c2c(-c3cccc4c3-c3ccc(N(c5ccccc5)c5ccc6c(ccc7ccccc76)c5)cc3C4(C)C)cccc21 ; CC1(C)c2cc(N(c3ccccc3)c3cccc(-c4ccccc4)c3)ccc2-c2c(-c3cccc4c3-c3ccc(N(c5ccccc5)c5ccc6c(ccc7ccccc76)c5)cc3C4(C)C)cccc21]

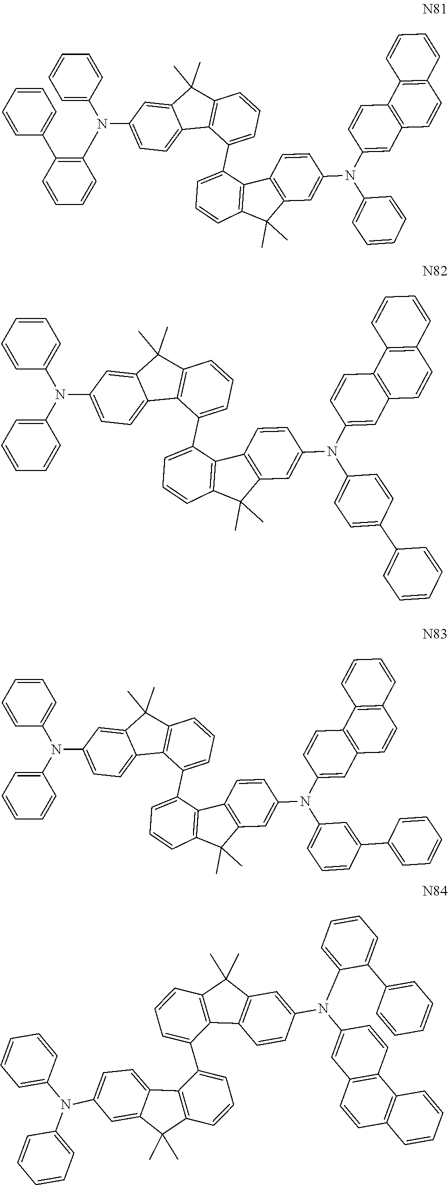 [SMILES: CC1(C)c2cc(N(c3ccccc3)c3ccc4c(ccc5ccccc54)c3)ccc2-c2c(-c3cccc4c3-c3ccc(N(c5ccccc5)c5ccccc5-c5ccccc5)cc3C4(C)C)cccc21 ; CC1(C)c2cc(N(c3ccccc3)c3ccccc3)ccc2-c2c(-c3cccc4c3-c3ccc(N(c5ccc(-c6ccccc6)cc5)c5ccc6c(ccc7ccccc76)c5)cc3C4(C)C)cccc21 ; CC1(C)c2cc(N(c3ccccc3)c3ccccc3)ccc2-c2c(-c3cccc4c3-c3ccc(N(c5cccc(-c6ccccc6)c5)c5ccc6c(ccc7ccccc76)c5)cc3C4(C)C)cccc21 ; CC1(C)c2cc(N(c3ccccc3)c3ccccc3)ccc2-c2c(-c3cccc4c3-c3ccc(N(c5ccc6c(ccc7ccccc76)c5)c5ccccc5-c5ccccc5)cc3C4(C)C)cccc21]

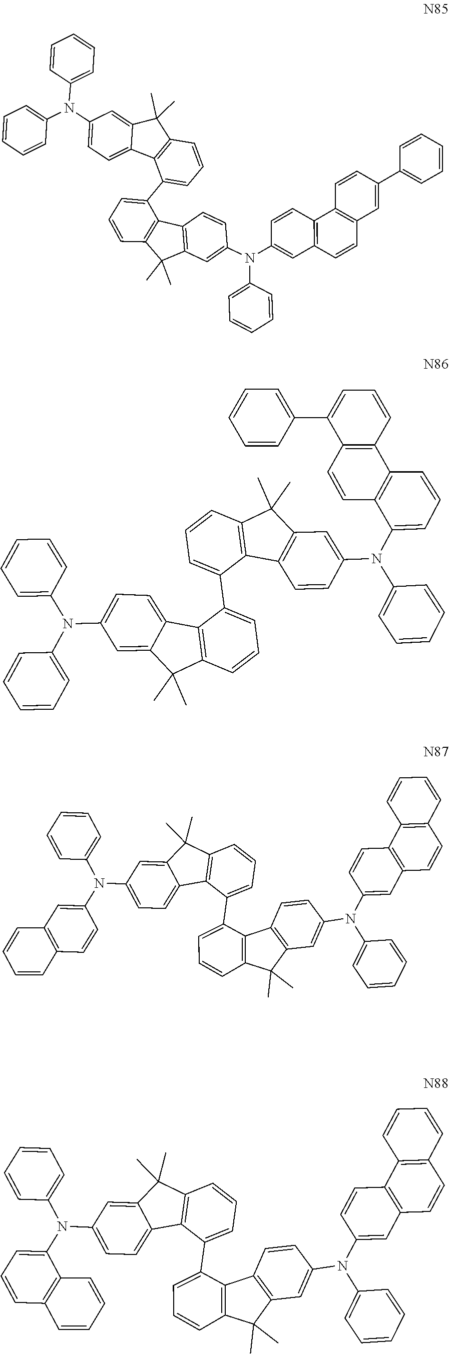 [SMILES: CC1(C)c2cc(N(c3ccccc3)c3ccccc3)ccc2-c2c(-c3cccc4c3-c3ccc(N(c5ccccc5)c5ccc6c(ccc7cc(-c8ccccc8)ccc76)c5)cc3C4(C)C)cccc21 ; CC1(C)c2cc(N(c3ccccc3)c3ccccc3)ccc2-c2c(-c3cccc4c3-c3ccc(N(c5ccccc5)c5cccc6c5ccc5c(-c7ccccc7)cccc56)cc3C4(C)C)cccc21 ; CC1(C)c2cc(N(c3ccccc3)c3ccc4ccccc4c3)ccc2-c2c(-c3cccc4c3-c3ccc(N(c5ccccc5)c5ccc6c(ccc7ccccc76)c5)cc3C4(C)C)cccc21 ; CC1(C)c2cc(N(c3ccccc3)c3ccc4c(ccc5ccccc54)c3)ccc2-c2c(-c3cccc4c3-c3ccc(N(c5ccccc5)c5cccc6ccccc56)cc3C4(C)C)cccc21]

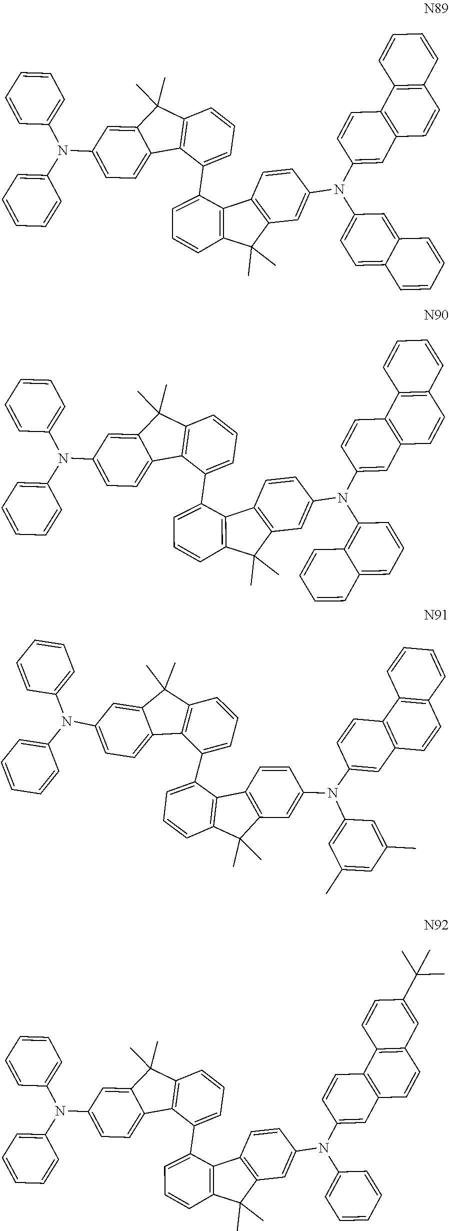 [SMILES: CC1(C)c2cc(N(c3ccccc3)c3ccccc3)ccc2-c2c(-c3cccc4c3-c3ccc(N(c5ccc6ccccc6c5)c5ccc6c(ccc7ccccc76)c5)cc3C4(C)C)cccc21 ; CC1(C)c2cc(N(c3ccccc3)c3ccccc3)ccc2-c2c(-c3cccc4c3-c3ccc(N(c5ccc6c(ccc7ccccc76)c5)c5cccc6ccccc56)cc3C4(C)C)cccc21 ; Cc1cc(C)cc(N(c2ccc3c(c2)C(C)(C)c2cccc(-c4cccc5c4-c4ccc(N(c6ccccc6)c6ccccc6)cc4C5(C)C)c2-3)c2ccc3c(ccc4ccccc43)c2)c1 ; CC(C)(C)c1ccc2c(ccc3cc(N(c4ccccc4)c4ccc5c(c4)C(C)(C)c4cccc(-c6cccc7c6-c6ccc(N(c8ccccc8)c8ccccc8)cc6C7(C)C)c4-5)ccc32)c1]

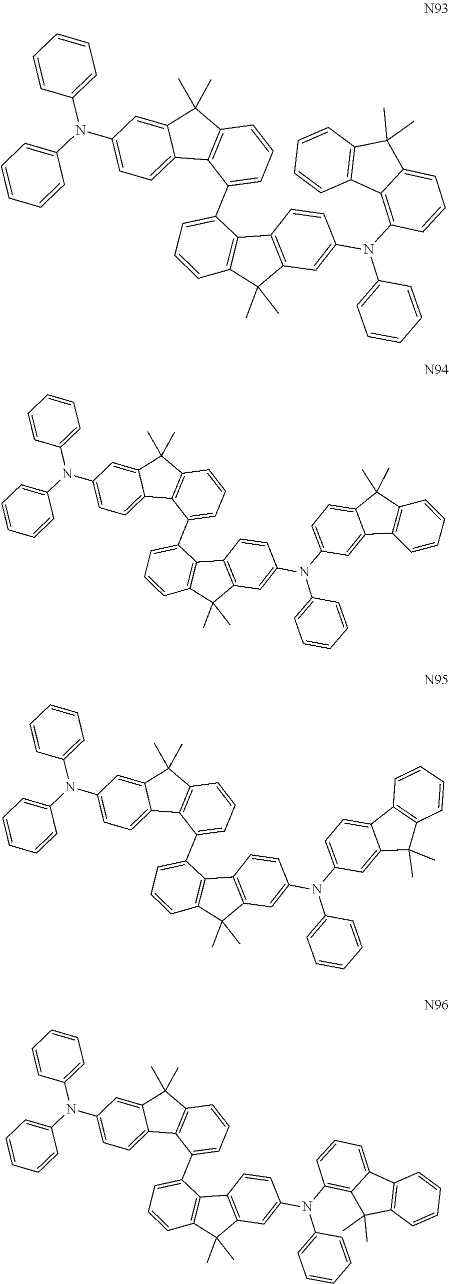 [SMILES: CC1(C)c2cc(N(c3ccccc3)c3ccccc3)ccc2-c2c(-c3cccc4c3-c3ccc(N(c5ccccc5)c5cccc6c5-c5ccccc5C6(C)C)cc3C4(C)C)cccc21 ; CC1(C)c2ccccc2-c2cc(N(c3ccccc3)c3ccc4c(c3)C(C)(C)c3cccc(-c5cccc6c5-c5ccc(N(c7ccccc7)c7ccccc7)cc5C6(C)C)c3-4)ccc21 ; CC1(C)c2ccccc2-c2ccc(N(c3ccccc3)c3ccc4c(c3)C(C)(C)c3cccc(-c5cccc6c5-c5ccc(N(c7ccccc7)c7ccccc7)cc5C6(C)C)c3-4)cc21 ; CC1(C)c2cc(N(c3ccccc3)c3ccccc3)ccc2-c2c(-c3cccc4c3-c3ccc(N(c5ccccc5)c5cccc6c5C(C)(C)c5ccccc5-6)cc3C4(C)C)cccc21]

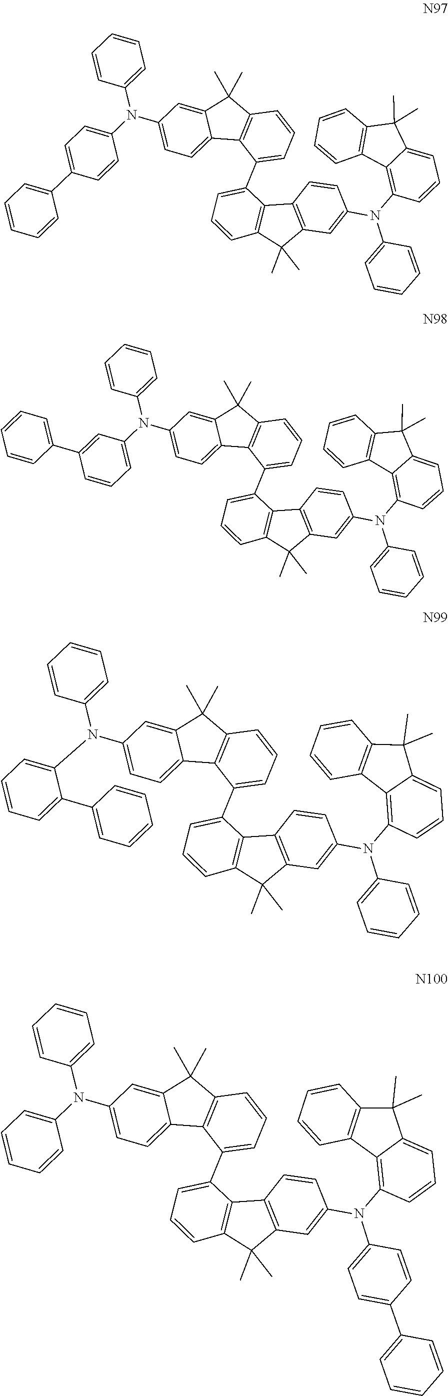 [SMILES: CC1(C)c2cc(N(c3ccccc3)c3ccc(-c4ccccc4)cc3)ccc2-c2c(-c3cccc4c3-c3ccc(N(c5ccccc5)c5cccc6c5-c5ccccc5C6(C)C)cc3C4(C)C)cccc21 ; CC1(C)c2cc(N(c3ccccc3)c3cccc(-c4ccccc4)c3)ccc2-c2c(-c3cccc4c3-c3ccc(N(c5ccccc5)c5cccc6c5-c5ccccc5C6(C)C)cc3C4(C)C)cccc21 ; CC1(C)c2cc(N(c3ccccc3)c3ccccc3-c3ccccc3)ccc2-c2c(-c3cccc4c3-c3ccc(N(c5ccccc5)c5cccc6c5-c5ccccc5C6(C)C)cc3C4(C)C)cccc21 ; CC1(C)c2cc(N(c3ccccc3)c3ccccc3)ccc2-c2c(-c3cccc4c3-c3ccc(N(c5ccc(-c6ccccc6)cc5)c5cccc6c5-c5ccccc5C6(C)C)cc3C4(C)C)cccc21]

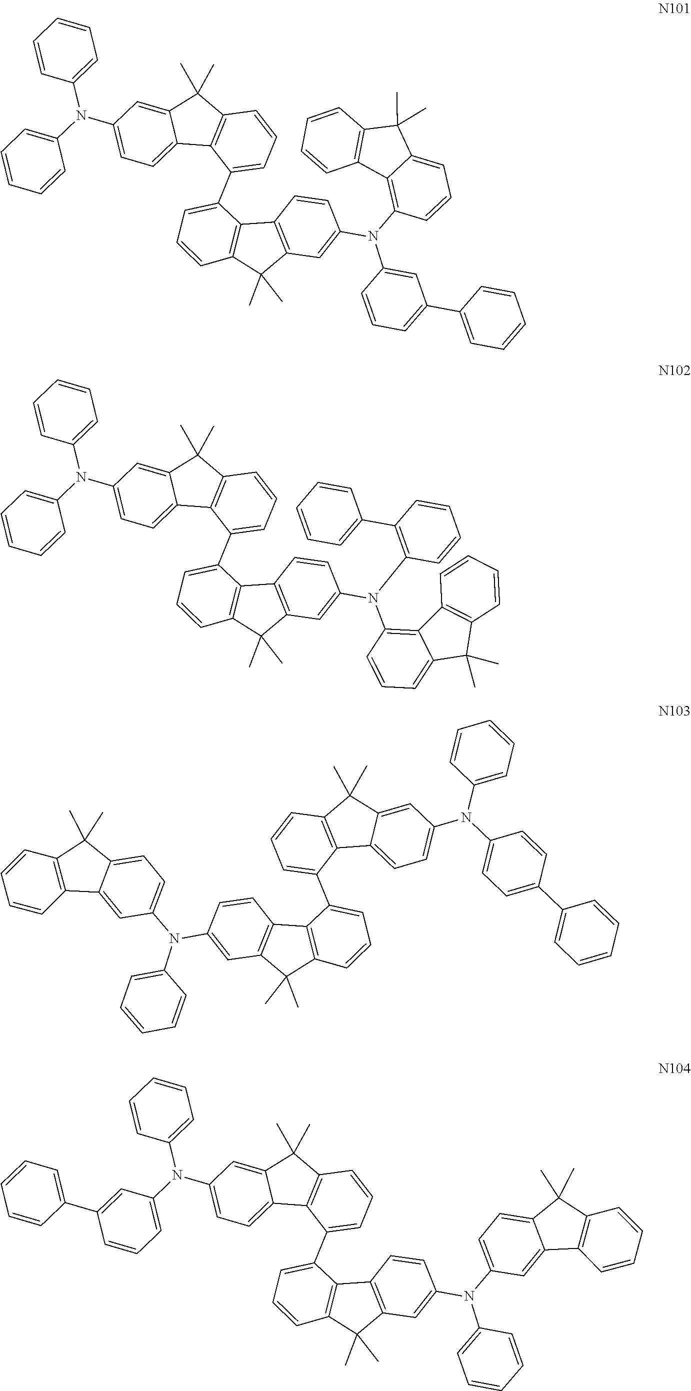

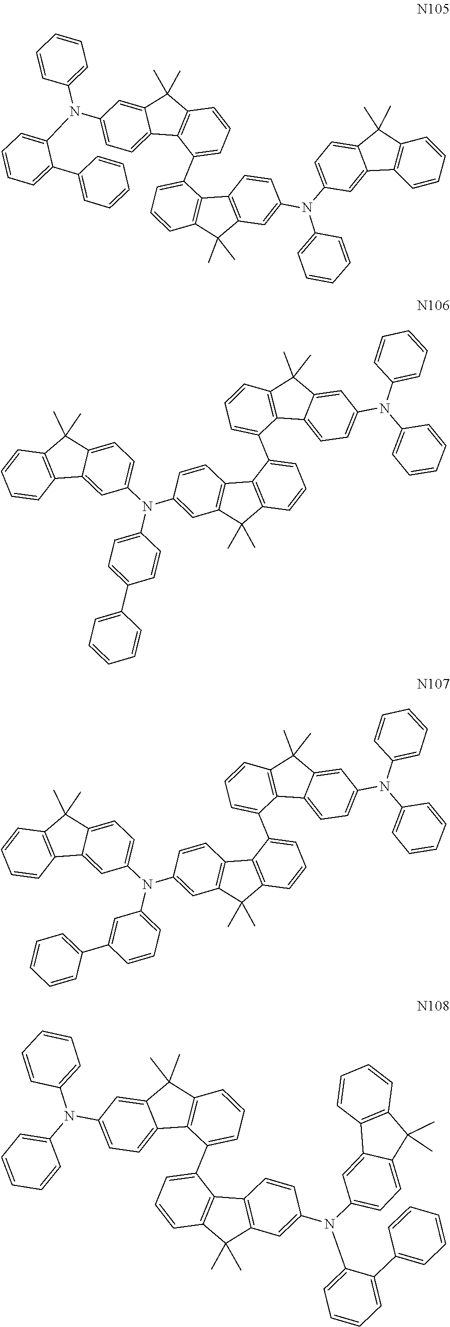

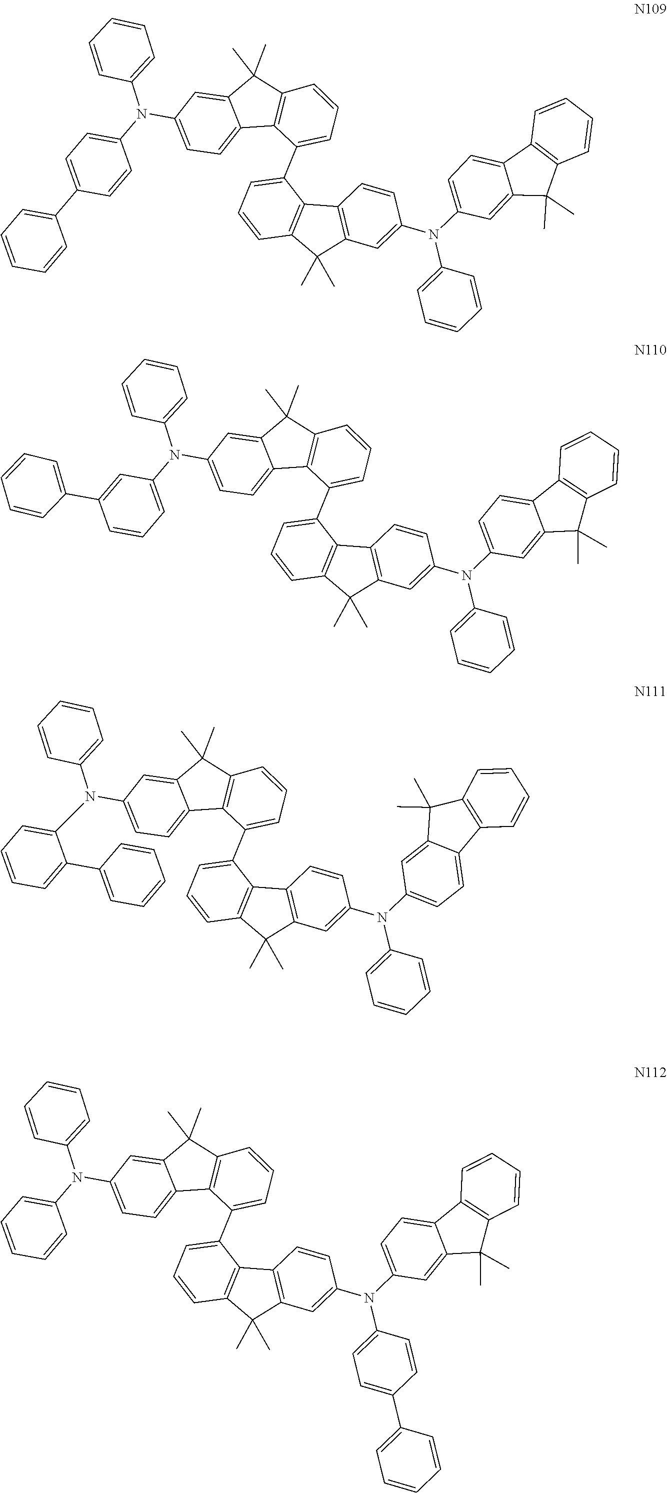

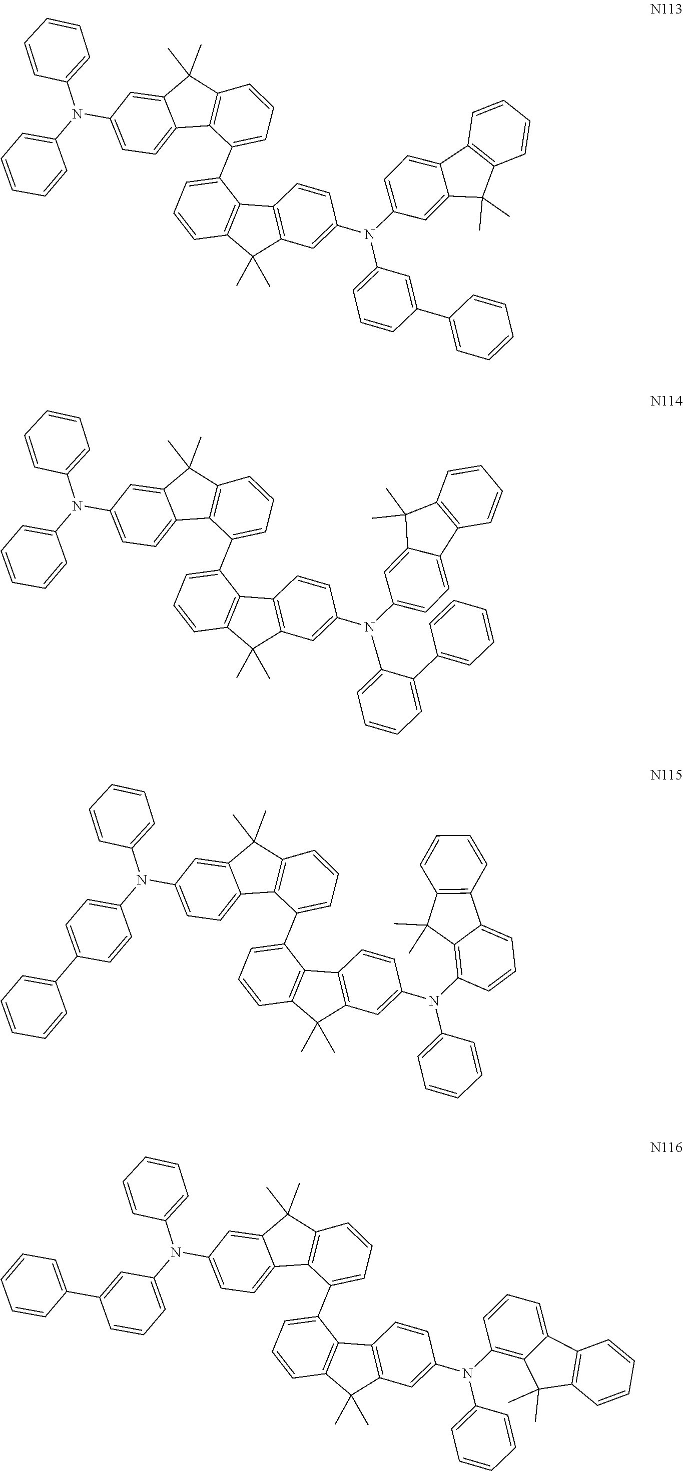 [SMILES: CC1(C)c2ccccc2-c2ccc(N(c3cccc(-c4ccccc4)c3)c3ccc4c(c3)C(C)(C)c3cccc(-c5cccc6c5-c5ccc(N(c7ccccc7)c7ccccc7)cc5C6(C)C)c3-4)cc21 ; CC1(C)c2ccccc2-c2ccc(N(c3ccc4c(c3)C(C)(C)c3cccc(-c5cccc6c5-c5ccc(N(c7ccccc7)c7ccccc7)cc5C6(C)C)c3-4)c3ccccc3-c3ccccc3)cc21 ; CC1(C)c2cc(N(c3ccccc3)c3ccc(-c4ccccc4)cc3)ccc2-c2c(-c3cccc4c3-c3ccc(N(c5ccccc5)c5cccc6c5C(C)(C)c5ccccc5-6)cc3C4(C)C)cccc21 ; CC1(C)c2cc(N(c3ccccc3)c3cccc(-c4ccccc4)c3)ccc2-c2c(-c3cccc4c3-c3ccc(N(c5ccccc5)c5cccc6c5C(C)(C)c5ccccc5-6)cc3C4(C)C)cccc21]

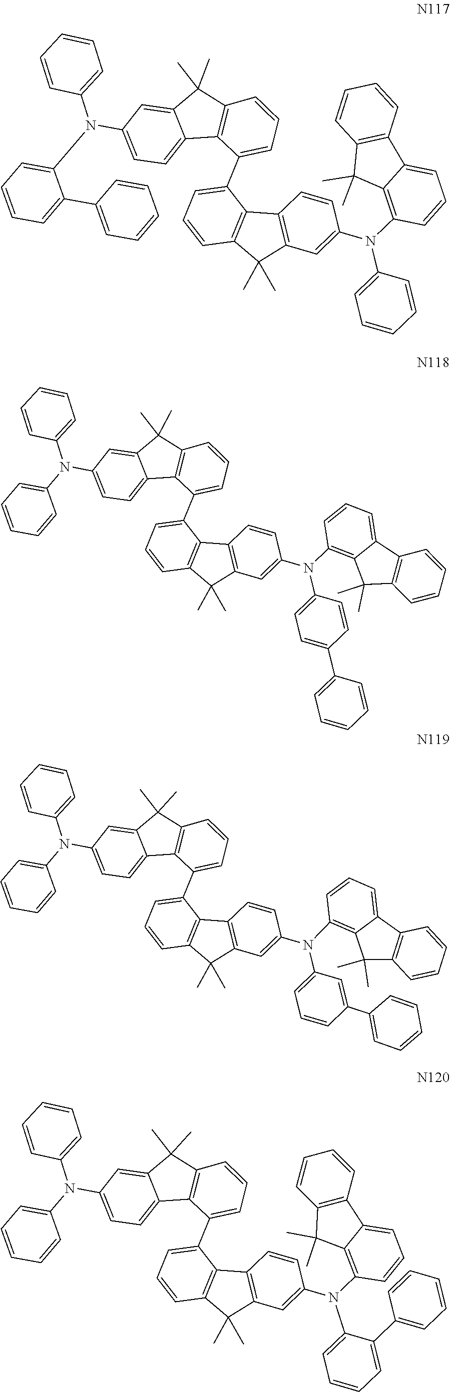 [SMILES: CC1(C)c2cc(N(c3ccccc3)c3ccccc3-c3ccccc3)ccc2-c2c(-c3cccc4c3-c3ccc(N(c5ccccc5)c5cccc6c5C(C)(C)c5ccccc5-6)cc3C4(C)C)cccc21 ; CC1(C)c2cc(N(c3ccccc3)c3ccccc3)ccc2-c2c(-c3cccc4c3-c3ccc(N(c5ccc(-c6ccccc6)cc5)c5cccc6c5C(C)(C)c5ccccc5-6)cc3C4(C)C)cccc21 ; CC1(C)c2cc(N(c3ccccc3)c3ccccc3)ccc2-c2c(-c3cccc4c3-c3ccc(N(c5cccc(-c6ccccc6)c5)c5cccc6c5C(C)(C)c5ccccc5-6)cc3C4(C)C)cccc21 ; CC1(C)c2cc(N(c3ccccc3)c3ccccc3)ccc2-c2c(-c3cccc4c3-c3ccc(N(c5ccccc5-c5ccccc5)c5cccc6c5C(C)(C)c5ccccc5-6)cc3C4(C)C)cccc21]

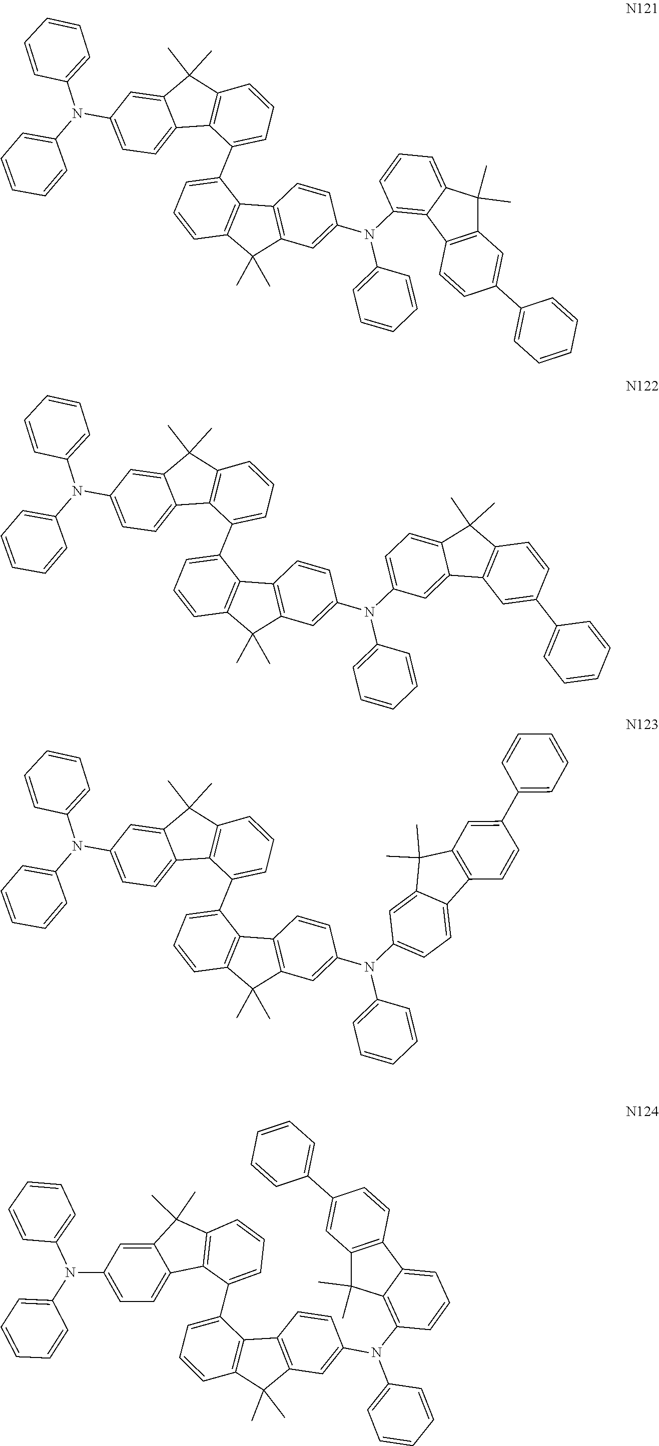 [SMILES: CC1(C)c2cc(N(c3ccccc3)c3ccccc3)ccc2-c2c(-c3cccc4c3-c3ccc(N(c5ccccc5)c5cccc6c5-c5ccc(-c7ccccc7)cc5C6(C)C)cc3C4(C)C)cccc21 ; CC1(C)c2ccc(-c3ccccc3)cc2-c2cc(N(c3ccccc3)c3ccc4c(c3)C(C)(C)c3cccc(-c5cccc6c5-c5ccc(N(c7ccccc7)c7ccccc7)cc5C6(C)C)c3-4)ccc21 ; CC1(C)c2cc(-c3ccccc3)ccc2-c2ccc(N(c3ccccc3)c3ccc4c(c3)C(C)(C)c3cccc(-c5cccc6c5-c5ccc(N(c7ccccc7)c7ccccc7)cc5C6(C)C)c3-4)cc21 ; CC1(C)c2cc(N(c3ccccc3)c3ccccc3)ccc2-c2c(-c3cccc4c3-c3ccc(N(c5ccccc5)c5cccc6c5C(C)(C)c5cc(-c7ccccc7)ccc5-6)cc3C4(C)C)cccc21]

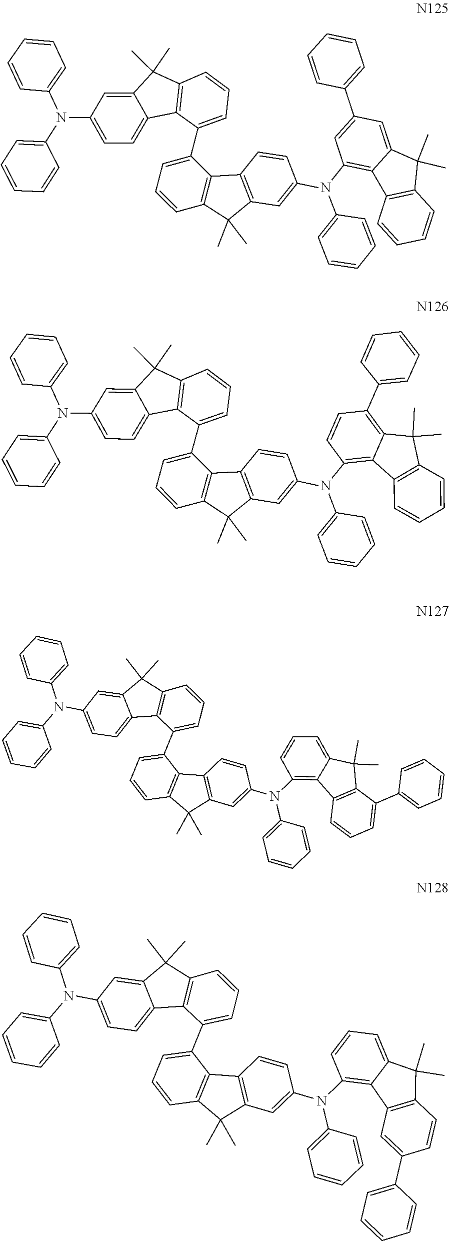 [SMILES: CC1(C)c2cc(N(c3ccccc3)c3ccccc3)ccc2-c2c(-c3cccc4c3-c3ccc(N(c5ccccc5)c5cc(-c6ccccc6)cc6c5-c5ccccc5C6(C)C)cc3C4(C)C)cccc21 ; CC1(C)c2cc(N(c3ccccc3)c3ccccc3)ccc2-c2c(-c3cccc4c3-c3ccc(N(c5ccccc5)c5ccc(-c6ccccc6)c6c5-c5ccccc5C6(C)C)cc3C4(C)C)cccc21 ; CC1(C)c2cc(N(c3ccccc3)c3ccccc3)ccc2-c2c(-c3cccc4c3-c3ccc(N(c5ccccc5)c5cccc6c5-c5cccc(-c7ccccc7)c5C6(C)C)cc3C4(C)C)cccc21 ; CC1(C)c2cc(N(c3ccccc3)c3ccccc3)ccc2-c2c(-c3cccc4c3-c3ccc(N(c5ccccc5)c5cccc6c5-c5cc(-c7ccccc7)ccc5C6(C)C)cc3C4(C)C)cccc21]

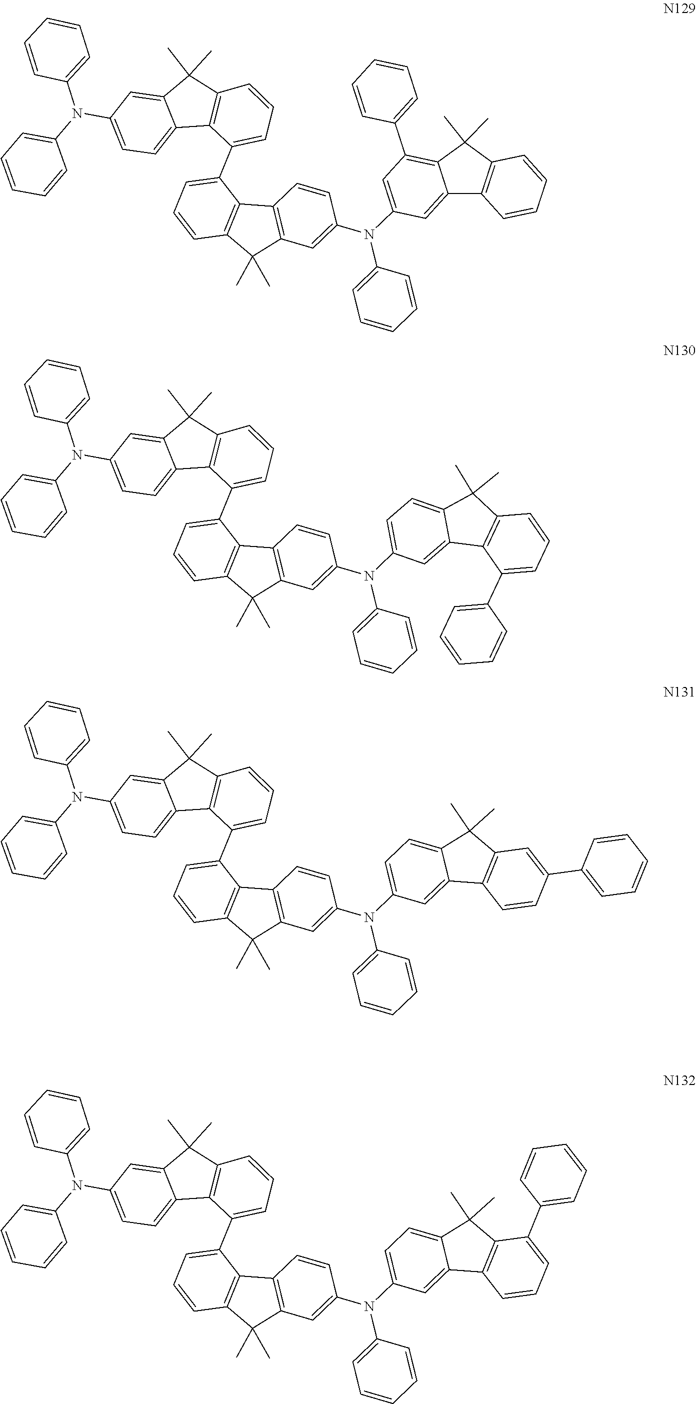

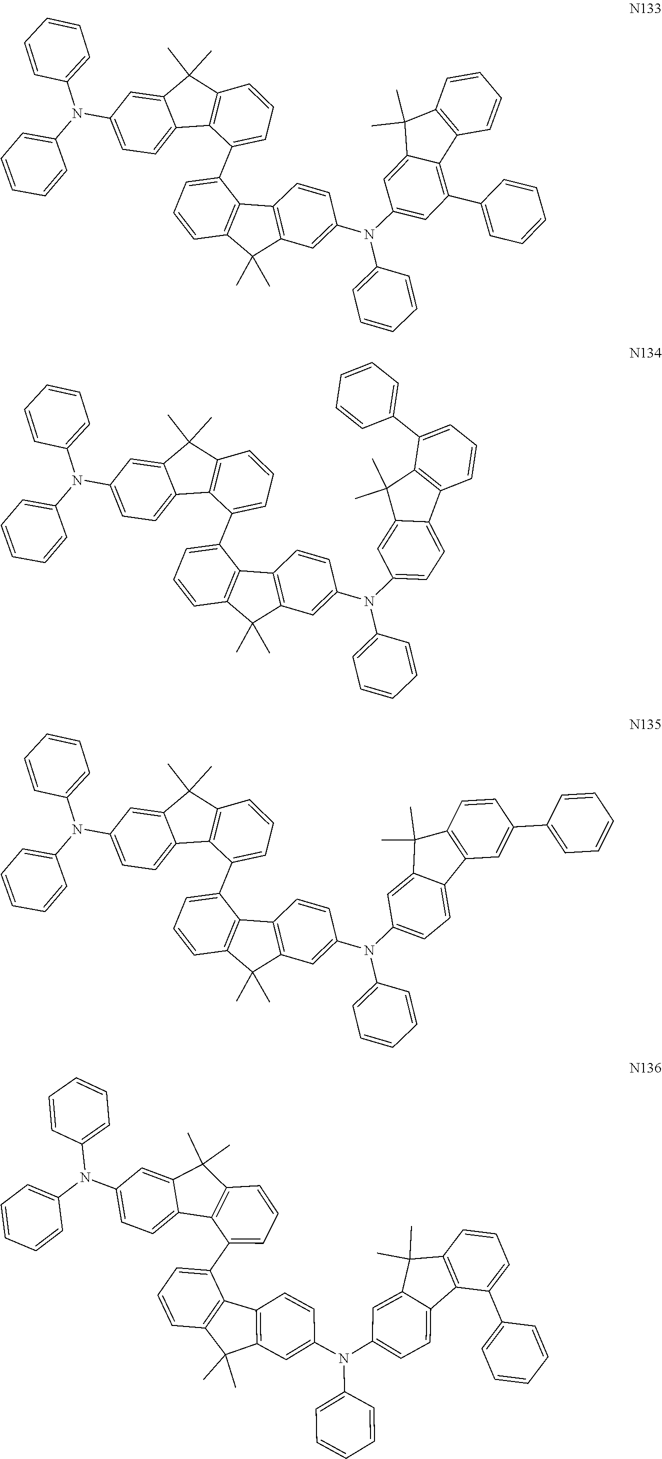

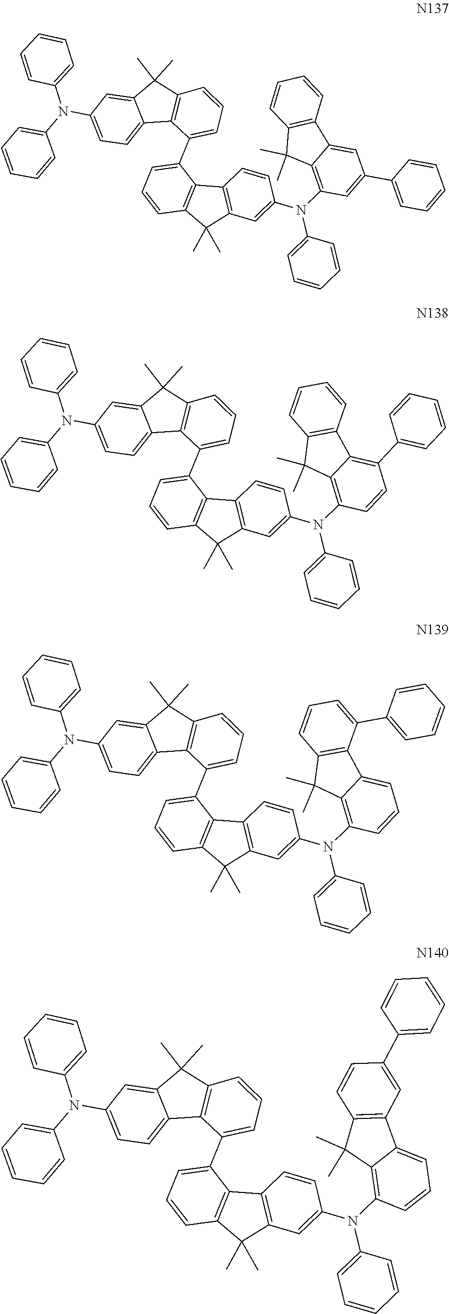 [SMILES: CC1(C)c2cc(N(c3ccccc3)c3ccccc3)ccc2-c2c(-c3cccc4c3-c3ccc(N(c5ccccc5)c5cc(-c6ccccc6)cc6c5C(C)(C)c5ccccc5-6)cc3C4(C)C)cccc21 ; CC1(C)c2cc(N(c3ccccc3)c3ccccc3)ccc2-c2c(-c3cccc4c3-c3ccc(N(c5ccccc5)c5ccc(-c6ccccc6)c6c5C(C)(C)c5ccccc5-6)cc3C4(C)C)cccc21 ; CC1(C)c2cc(N(c3ccccc3)c3ccccc3)ccc2-c2c(-c3cccc4c3-c3ccc(N(c5ccccc5)c5cccc6c5C(C)(C)c5cccc(-c7ccccc7)c5-6)cc3C4(C)C)cccc21 ; CC1(C)c2cc(N(c3ccccc3)c3ccccc3)ccc2-c2c(-c3cccc4c3-c3ccc(N(c5ccccc5)c5cccc6c5C(C)(C)c5ccc(-c7ccccc7)cc5-6)cc3C4(C)C)cccc21]

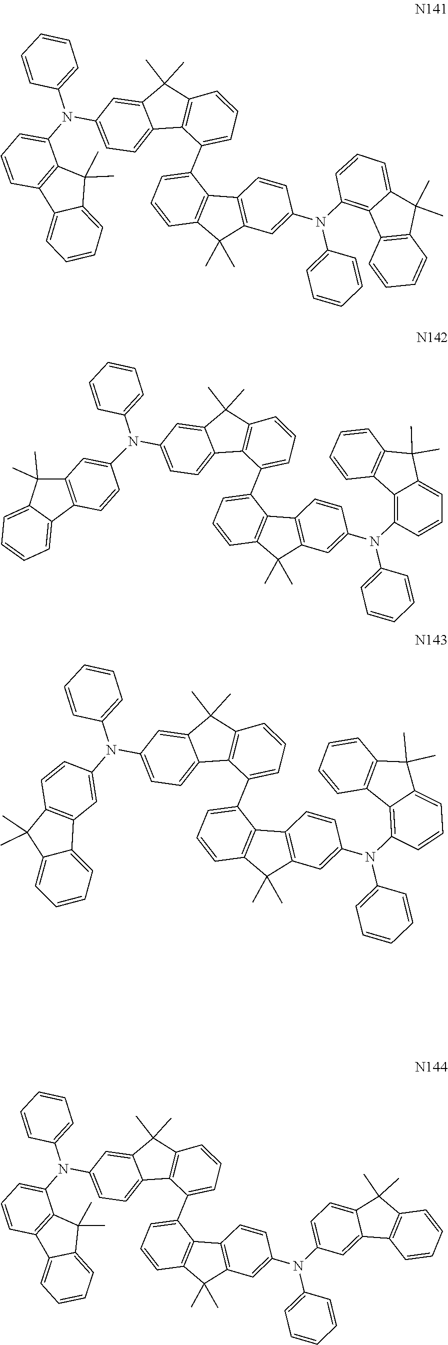

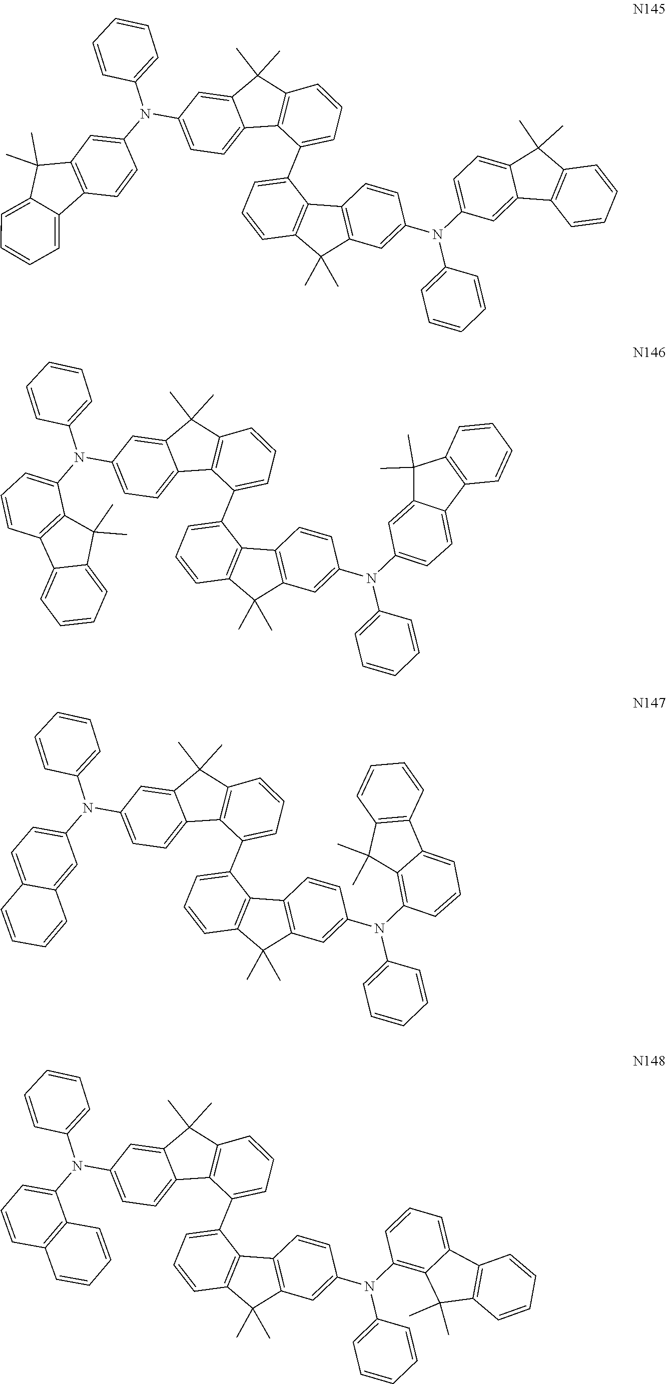

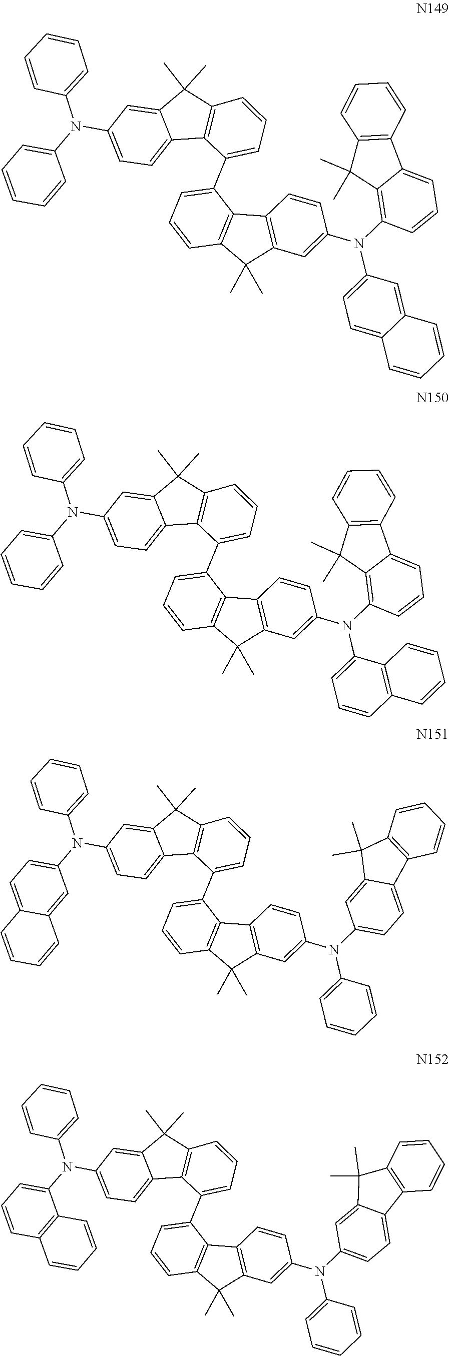 [SMILES: CC1(C)c2cc(N(c3ccccc3)c3ccccc3)ccc2-c2c(-c3cccc4c3-c3ccc(N(c5ccc6ccccc6c5)c5cccc6c5C(C)(C)c5ccccc5-6)cc3C4(C)C)cccc21 ; CC1(C)c2cc(N(c3ccccc3)c3ccccc3)ccc2-c2c(-c3cccc4c3-c3ccc(N(c5cccc6c5C(C)(C)c5ccccc5-6)c5cccc6ccccc56)cc3C4(C)C)cccc21 ; CC1(C)c2ccccc2-c2ccc(N(c3ccccc3)c3ccc4c(c3)C(C)(C)c3cccc(-c5cccc6c5-c5ccc(N(c7ccccc7)c7ccc8ccccc8c7)cc5C6(C)C)c3-4)cc21 ; CC1(C)c2ccccc2-c2ccc(N(c3ccccc3)c3ccc4c(c3)C(C)(C)c3cccc(-c5cccc6c5-c5ccc(N(c7ccccc7)c7cccc8ccccc78)cc5C6(C)C)c3-4)cc21]

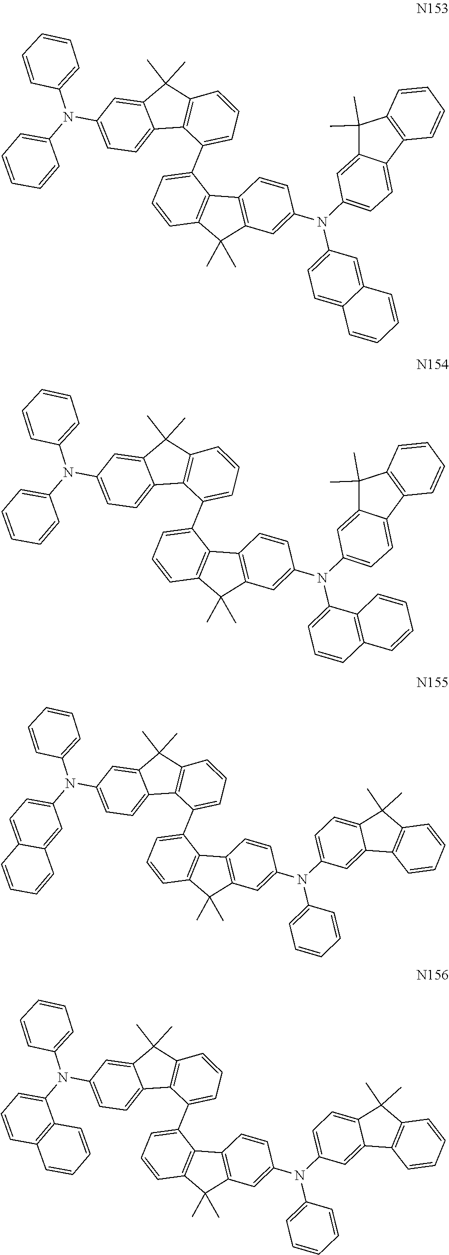 [SMILES: CC1(C)c2ccccc2-c2ccc(N(c3ccc4c(c3)C(C)(C)c3cccc(-c5cccc6c5-c5ccc(N(c7ccccc7)c7ccccc7)cc5C6(C)C)c3-4)c3ccc4ccccc4c3)cc21 ; CC1(C)c2ccccc2-c2ccc(N(c3ccc4c(c3)C(C)(C)c3cccc(-c5cccc6c5-c5ccc(N(c7ccccc7)c7ccccc7)cc5C6(C)C)c3-4)c3cccc4ccccc34)cc21 ; CC1(C)c2ccccc2-c2cc(N(c3ccccc3)c3ccc4c(c3)C(C)(C)c3cccc(-c5cccc6c5-c5ccc(N(c7ccccc7)c7ccc8ccccc8c7)cc5C6(C)C)c3-4)ccc21 ; CC1(C)c2ccccc2-c2cc(N(c3ccccc3)c3ccc4c(c3)C(C)(C)c3cccc(-c5cccc6c5-c5ccc(N(c7ccccc7)c7cccc8ccccc78)cc5C6(C)C)c3-4)ccc21]

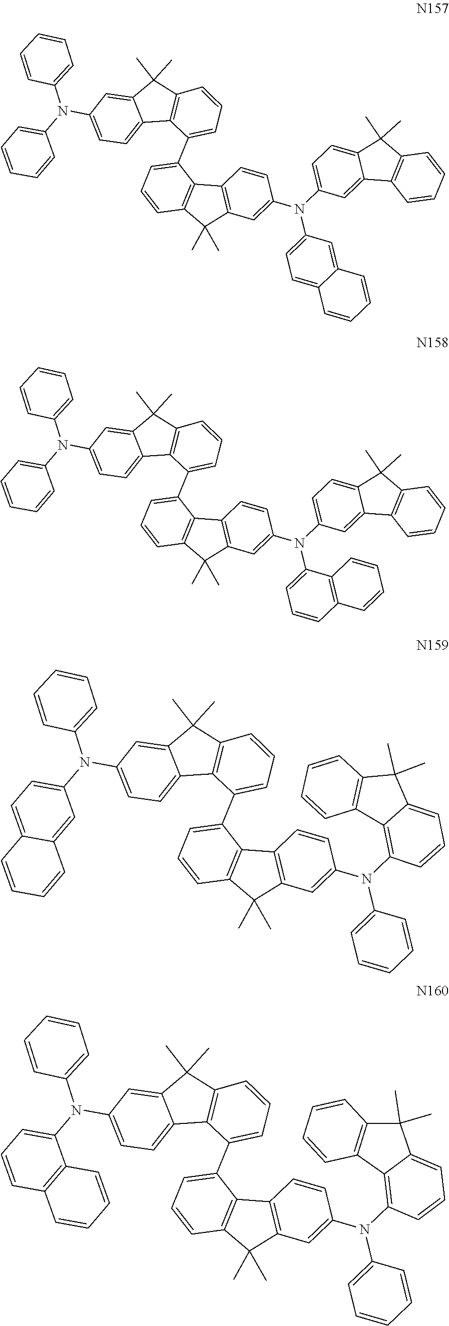 [SMILES: CC1(C)c2ccccc2-c2cc(N(c3ccc4c(c3)C(C)(C)c3cccc(-c5cccc6c5-c5ccc(N(c7ccccc7)c7ccccc7)cc5C6(C)C)c3-4)c3ccc4ccccc4c3)ccc21 ; CC1(C)c2ccccc2-c2cc(N(c3ccc4c(c3)C(C)(C)c3cccc(-c5cccc6c5-c5ccc(N(c7ccccc7)c7ccccc7)cc5C6(C)C)c3-4)c3cccc4ccccc34)ccc21 ; CC1(C)c2cc(N(c3ccccc3)c3ccc4ccccc4c3)ccc2-c2c(-c3cccc4c3-c3ccc(N(c5ccccc5)c5cccc6c5-c5ccccc5C6(C)C)cc3C4(C)C)cccc21 ; CC1(C)c2cc(N(c3ccccc3)c3cccc4c3-c3ccccc3C4(C)C)ccc2-c2c(-c3cccc4c3-c3ccc(N(c5ccccc5)c5cccc6ccccc56)cc3C4(C)C)cccc21]

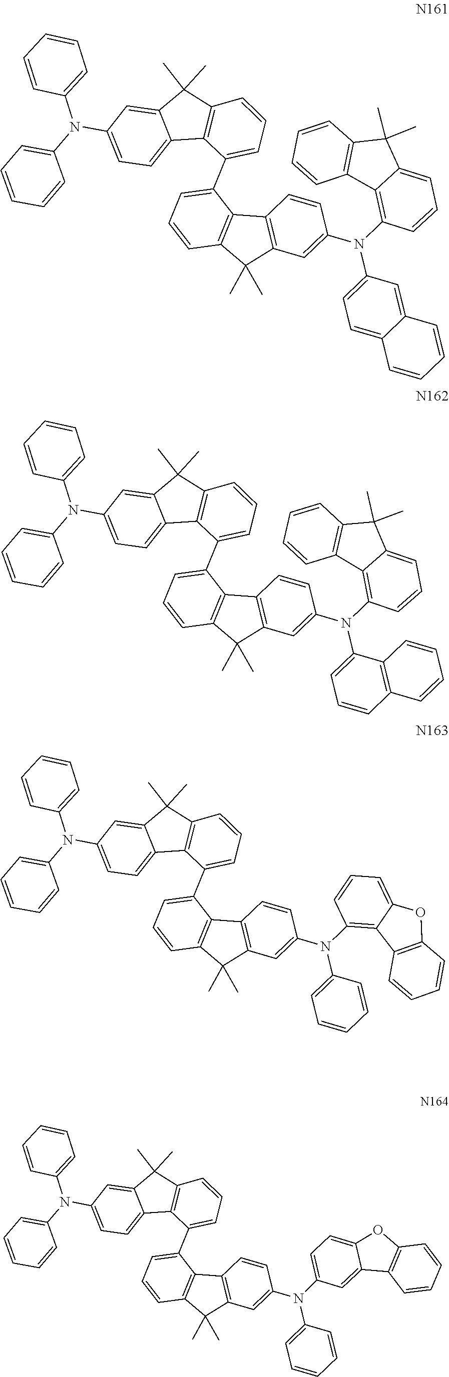 [SMILES: CC1(C)c2cc(N(c3ccccc3)c3ccccc3)ccc2-c2c(-c3cccc4c3-c3ccc(N(c5ccc6ccccc6c5)c5cccc6c5-c5ccccc5C6(C)C)cc3C4(C)C)cccc21 ; CC1(C)c2cc(N(c3ccccc3)c3ccccc3)ccc2-c2c(-c3cccc4c3-c3ccc(N(c5cccc6c5-c5ccccc5C6(C)C)c5cccc6ccccc56)cc3C4(C)C)cccc21 ; CC1(C)c2cc(N(c3ccccc3)c3ccccc3)ccc2-c2c(-c3cccc4c3-c3ccc(N(c5ccccc5)c5cccc6oc7ccccc7c56)cc3C4(C)C)cccc21 ; CC1(C)c2cc(N(c3ccccc3)c3ccccc3)ccc2-c2c(-c3cccc4c3-c3ccc(N(c5ccccc5)c5ccc6oc7ccccc7c6c5)cc3C4(C)C)cccc21]

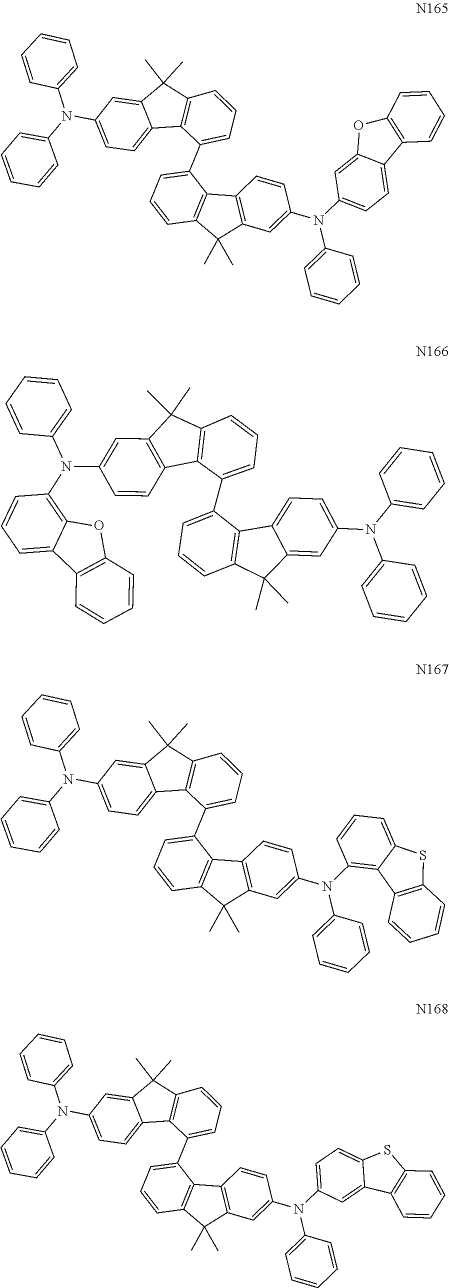 [SMILES: CC1(C)c2cc(N(c3ccccc3)c3ccccc3)ccc2-c2c(-c3cccc4c3-c3ccc(N(c5ccccc5)c5ccc6c(c5)oc5ccccc56)cc3C4(C)C)cccc21 ; CC1(C)c2cc(N(c3ccccc3)c3ccccc3)ccc2-c2c(-c3cccc4c3-c3ccc(N(c5ccccc5)c5cccc6c5oc5ccccc56)cc3C4(C)C)cccc21 ; CC1(C)c2cc(N(c3ccccc3)c3ccccc3)ccc2-c2c(-c3cccc4c3-c3ccc(N(c5ccccc5)c5cccc6sc7ccccc7c56)cc3C4(C)C)cccc21 ; CC1(C)c2cc(N(c3ccccc3)c3ccccc3)ccc2-c2c(-c3cccc4c3-c3ccc(N(c5ccccc5)c5ccc6sc7ccccc7c6c5)cc3C4(C)C)cccc21]

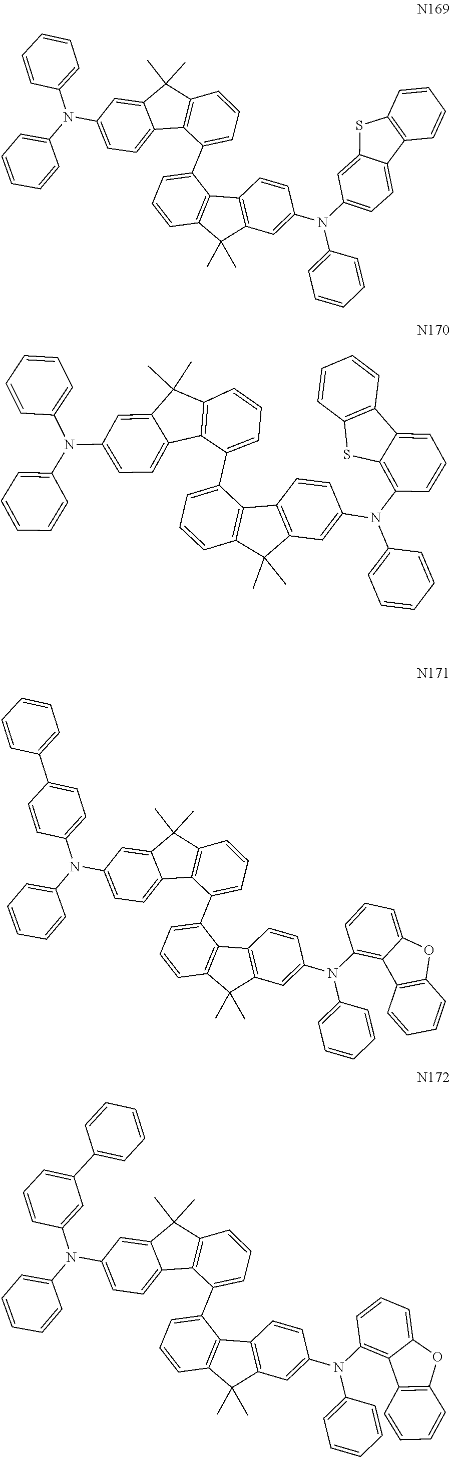 [SMILES: CC1(C)c2cc(N(c3ccccc3)c3ccccc3)ccc2-c2c(-c3cccc4c3-c3ccc(N(c5ccccc5)c5ccc6c(c5)sc5ccccc56)cc3C4(C)C)cccc21 ; CC1(C)c2cc(N(c3ccccc3)c3ccccc3)ccc2-c2c(-c3cccc4c3-c3ccc(N(c5ccccc5)c5cccc6c5sc5ccccc56)cc3C4(C)C)cccc21 ; CC1(C)c2cc(N(c3ccccc3)c3ccc(-c4ccccc4)cc3)ccc2-c2c(-c3cccc4c3-c3ccc(N(c5ccccc5)c5cccc6oc7ccccc7c56)cc3C4(C)C)cccc21 ; CC1(C)c2cc(N(c3ccccc3)c3cccc(-c4ccccc4)c3)ccc2-c2c(-c3cccc4c3-c3ccc(N(c5ccccc5)c5cccc6oc7ccccc7c56)cc3C4(C)C)cccc21]

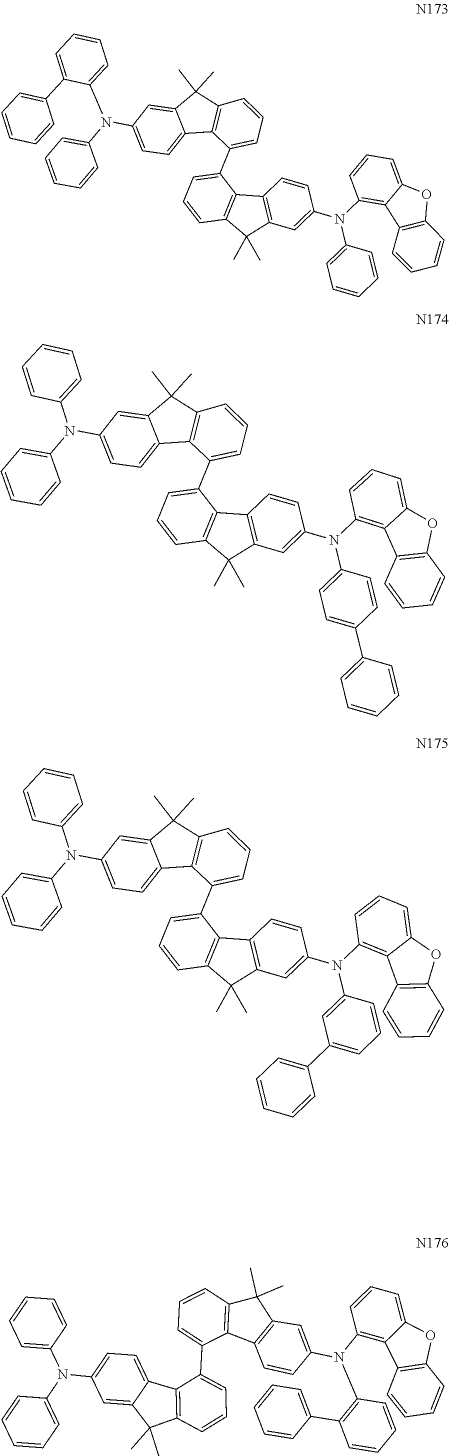 [SMILES: CC1(C)c2cc(N(c3ccccc3)c3ccccc3-c3ccccc3)ccc2-c2c(-c3cccc4c3-c3ccc(N(c5ccccc5)c5cccc6oc7ccccc7c56)cc3C4(C)C)cccc21 ; CC1(C)c2cc(N(c3ccccc3)c3ccccc3)ccc2-c2c(-c3cccc4c3-c3ccc(N(c5ccc(-c6ccccc6)cc5)c5cccc6oc7ccccc7c56)cc3C4(C)C)cccc21 ; CC1(C)c2cc(N(c3ccccc3)c3ccccc3)ccc2-c2c(-c3cccc4c3-c3ccc(N(c5cccc(-c6ccccc6)c5)c5cccc6oc7ccccc7c56)cc3C4(C)C)cccc21 ; CC1(C)c2cc(N(c3ccccc3)c3ccccc3)ccc2-c2c(-c3cccc4c3-c3ccc(N(c5ccccc5-c5ccccc5)c5cccc6oc7ccccc7c56)cc3C4(C)C)cccc21]

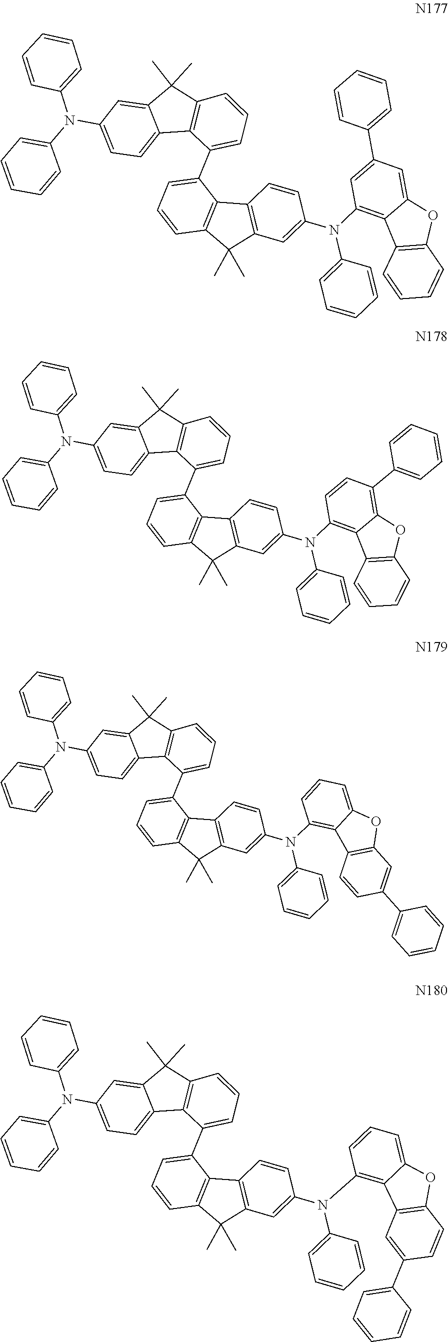 [SMILES: CC1(C)c2cc(N(c3ccccc3)c3ccccc3)ccc2-c2c(-c3cccc4c3-c3ccc(N(c5ccccc5)c5cc(-c6ccccc6)cc6oc7ccccc7c56)cc3C4(C)C)cccc21 ; CC1(C)c2cc(N(c3ccccc3)c3ccccc3)ccc2-c2c(-c3cccc4c3-c3ccc(N(c5ccccc5)c5ccc(-c6ccccc6)c6oc7ccccc7c56)cc3C4(C)C)cccc21 ; CC1(C)c2cc(N(c3ccccc3)c3ccccc3)ccc2-c2c(-c3cccc4c3-c3ccc(N(c5ccccc5)c5cccc6oc7cc(-c8ccccc8)ccc7c56)cc3C4(C)C)cccc21 ; CC1(C)c2cc(N(c3ccccc3)c3ccccc3)ccc2-c2c(-c3cccc4c3-c3ccc(N(c5ccccc5)c5cccc6oc7ccc(-c8ccccc8)cc7c56)cc3C4(C)C)cccc21]

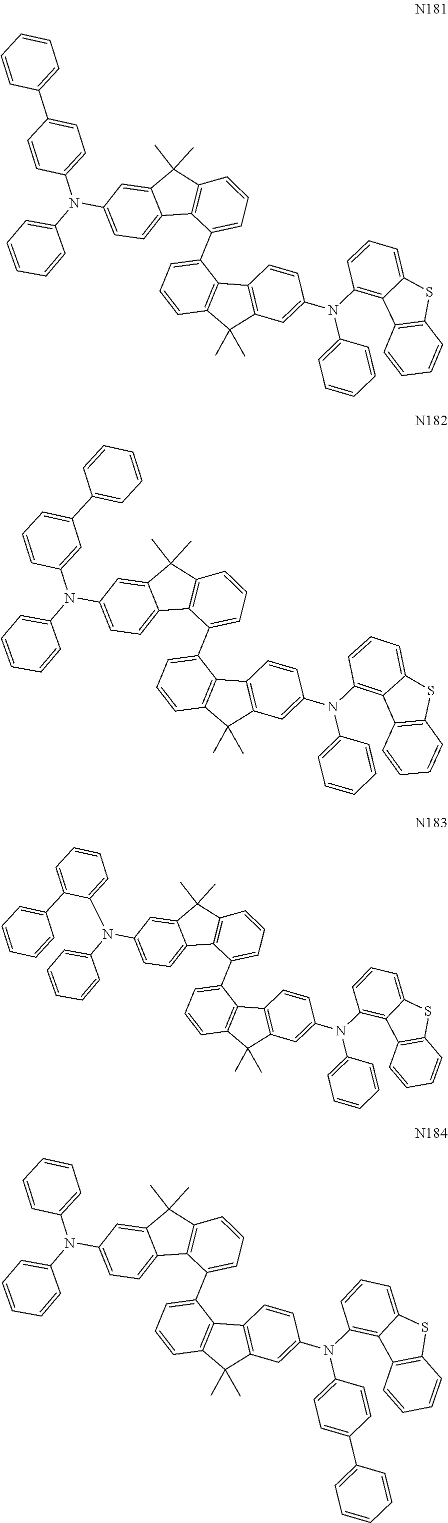 [SMILES: CC1(C)c2cc(N(c3ccccc3)c3ccc(-c4ccccc4)cc3)ccc2-c2c(-c3cccc4c3-c3ccc(N(c5ccccc5)c5cccc6sc7ccccc7c56)cc3C4(C)C)cccc21 ; CC1(C)c2cc(N(c3ccccc3)c3cccc(-c4ccccc4)c3)ccc2-c2c(-c3cccc4c3-c3ccc(N(c5ccccc5)c5cccc6sc7ccccc7c56)cc3C4(C)C)cccc21 ; CC1(C)c2cc(N(c3ccccc3)c3ccccc3-c3ccccc3)ccc2-c2c(-c3cccc4c3-c3ccc(N(c5ccccc5)c5cccc6sc7ccccc7c56)cc3C4(C)C)cccc21 ; CC1(C)c2cc(N(c3ccccc3)c3ccccc3)ccc2-c2c(-c3cccc4c3-c3ccc(N(c5ccc(-c6ccccc6)cc5)c5cccc6sc7ccccc7c56)cc3C4(C)C)cccc21]

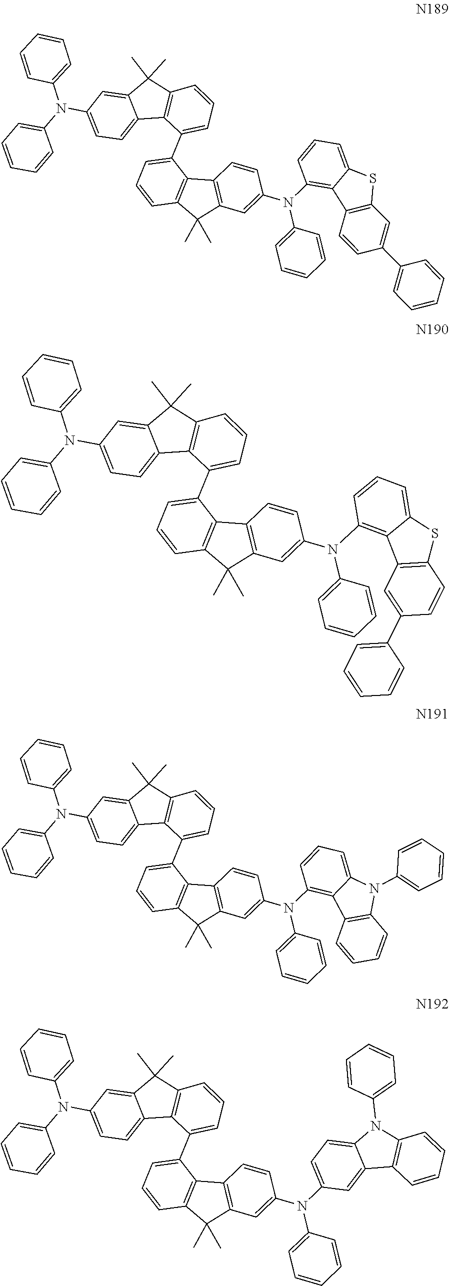 [SMILES: CC1(C)c2cc(N(c3ccccc3)c3ccccc3)ccc2-c2c(-c3cccc4c3-c3ccc(N(c5ccccc5)c5cccc6sc7cc(-c8ccccc8)ccc7c56)cc3C4(C)C)cccc21 ; CC1(C)c2cc(N(c3ccccc3)c3ccccc3)ccc2-c2c(-c3cccc4c3-c3ccc(N(c5ccccc5)c5cccc6sc7ccc(-c8ccccc8)cc7c56)cc3C4(C)C)cccc21 ; CC1(C)c2cc(N(c3ccccc3)c3ccccc3)ccc2-c2c(-c3cccc4c3-c3ccc(N(c5ccccc5)c5cccc6c5c5ccccc5n6-c5ccccc5)cc3C4(C)C)cccc21 ; CC1(C)c2cc(N(c3ccccc3)c3ccccc3)ccc2-c2c(-c3cccc4c3-c3ccc(N(c5ccccc5)c5ccc6c(c5)c5ccccc5n6-c5ccccc5)cc3C4(C)C)cccc21]

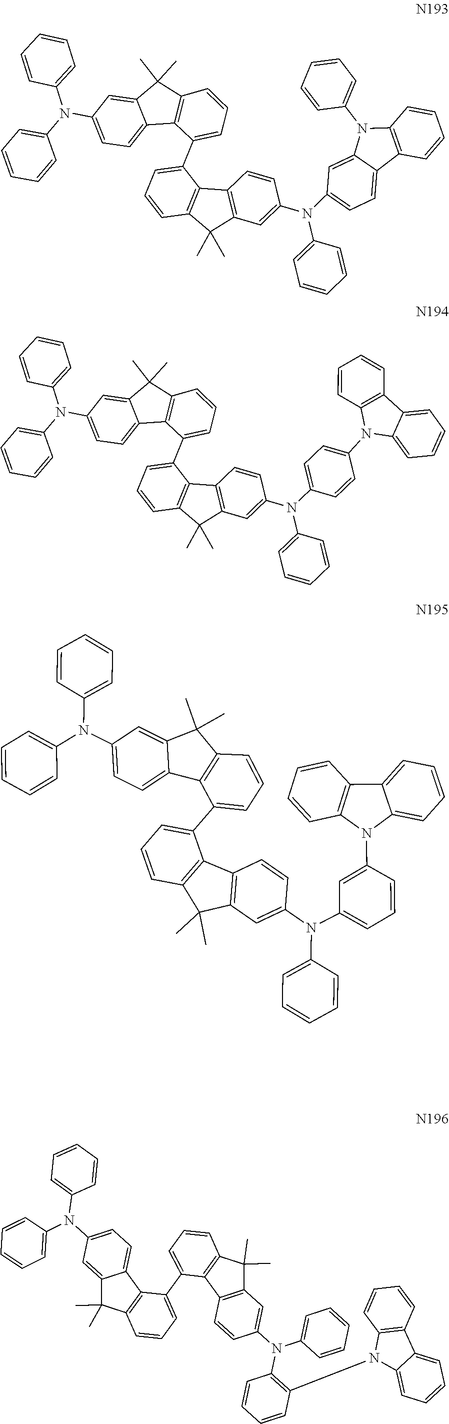 [SMILES: CC1(C)c2cc(N(c3ccccc3)c3ccccc3)ccc2-c2c(-c3cccc4c3-c3ccc(N(c5ccccc5)c5ccc6c7ccccc7n(-c7ccccc7)c6c5)cc3C4(C)C)cccc21 ; CC1(C)c2cc(N(c3ccccc3)c3ccccc3)ccc2-c2c(-c3cccc4c3-c3ccc(N(c5ccccc5)c5ccc(-n6c7ccccc7c7ccccc76)cc5)cc3C4(C)C)cccc21 ; CC1(C)c2cc(N(c3ccccc3)c3ccccc3)ccc2-c2c(-c3cccc4c3-c3ccc(N(c5ccccc5)c5cccc(-n6c7ccccc7c7ccccc76)c5)cc3C4(C)C)cccc21 ; CC1(C)c2cc(N(c3ccccc3)c3ccccc3)ccc2-c2c(-c3cccc4c3-c3ccc(N(c5ccccc5)c5ccccc5-n5c6ccccc6c6ccccc65)cc3C4(C)C)cccc21]

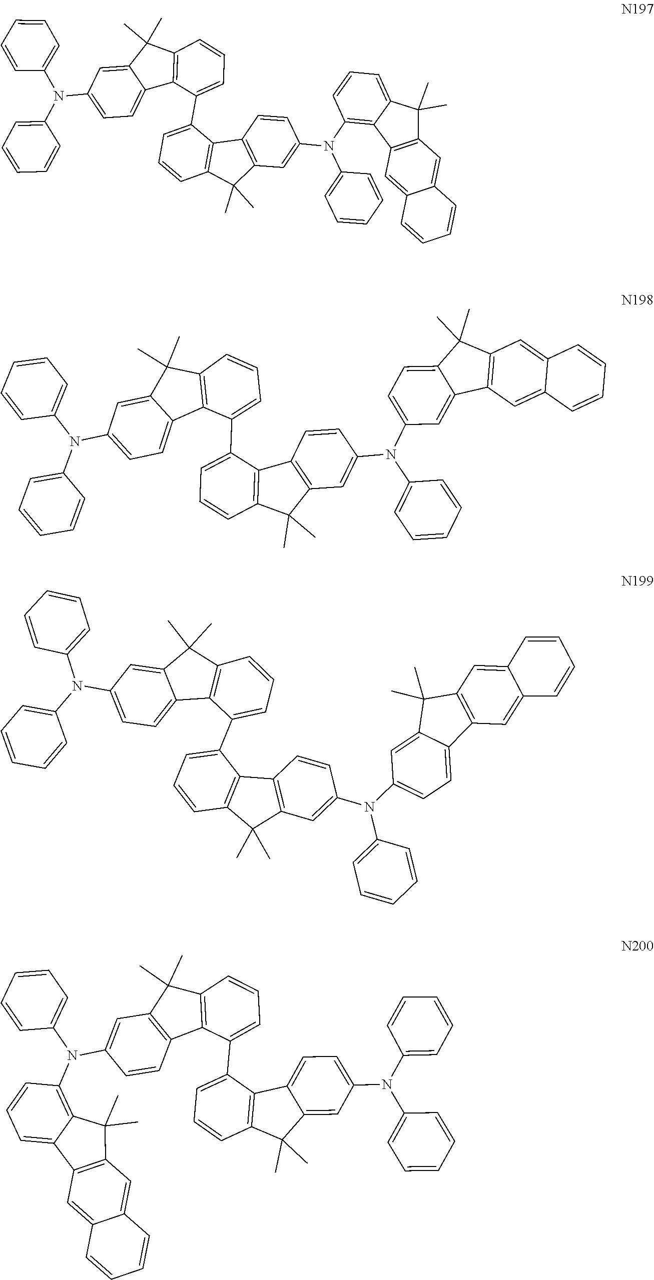 [SMILES: CC1(C)c2cc(N(c3ccccc3)c3ccccc3)ccc2-c2c(-c3cccc4c3-c3ccc(N(c5ccccc5)c5cccc6c5-c5cc7ccccc7cc5C6(C)C)cc3C4(C)C)cccc21 ; CC1(C)c2ccc(N(c3ccccc3)c3ccc4c(c3)C(C)(C)c3cccc(-c5cccc6c5-c5ccc(N(c7ccccc7)c7ccccc7)cc5C6(C)C)c3-4)cc2-c2cc3ccccc3cc21 ; CC1(C)c2cc(N(c3ccccc3)c3ccc4c(c3)C(C)(C)c3cccc(-c5cccc6c5-c5ccc(N(c7ccccc7)c7ccccc7)cc5C6(C)C)c3-4)ccc2-c2cc3ccccc3cc21 ; CC1(C)c2cc(N(c3ccccc3)c3ccccc3)ccc2-c2c(-c3cccc4c3-c3ccc(N(c5ccccc5)c5cccc6c5C(C)(C)c5cc7ccccc7cc5-6)cc3C4(C)C)cccc21]

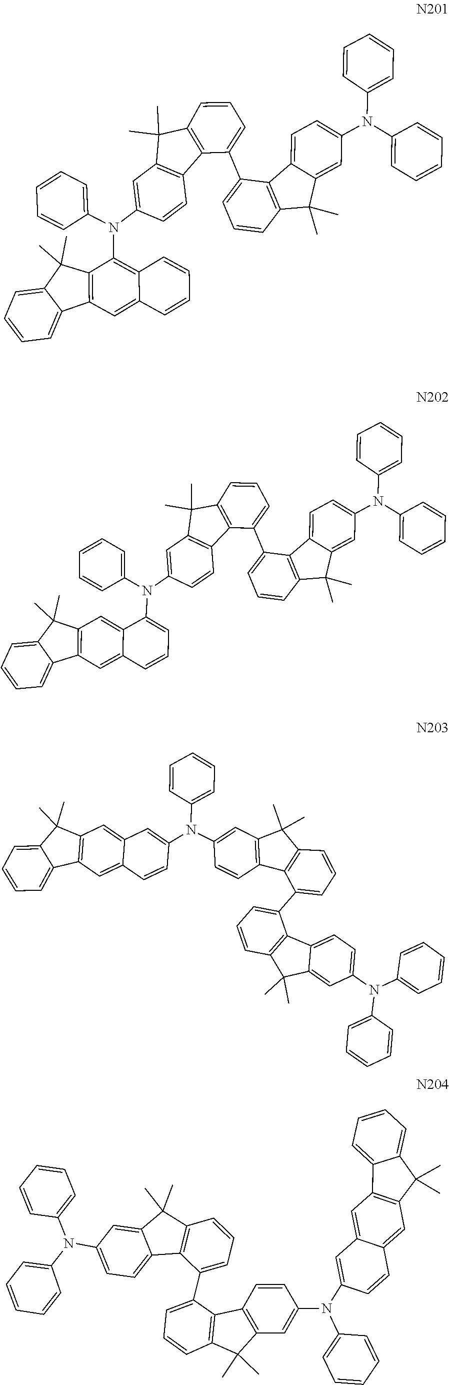 [SMILES: CC1(C)c2cc(N(c3ccccc3)c3ccccc3)ccc2-c2c(-c3cccc4c3-c3ccc(N(c5ccccc5)c5c6c(cc7ccccc57)-c5ccccc5C6(C)C)cc3C4(C)C)cccc21 ; CC1(C)c2ccccc2-c2cc3cccc(N(c4ccccc4)c4ccc5c(c4)C(C)(C)c4cccc(-c6cccc7c6-c6ccc(N(c8ccccc8)c8ccccc8)cc6C7(C)C)c4-5)c3cc21 ; CC1(C)c2ccccc2-c2cc3ccc(N(c4ccccc4)c4ccc5c(c4)C(C)(C)c4cccc(-c6cccc7c6-c6ccc(N(c8ccccc8)c8ccccc8)cc6C7(C)C)c4-5)cc3cc21 ; CC1(C)c2ccccc2-c2cc3cc(N(c4ccccc4)c4ccc5c(c4)C(C)(C)c4cccc(-c6cccc7c6-c6ccc(N(c8ccccc8)c8ccccc8)cc6C7(C)C)c4-5)ccc3cc21]

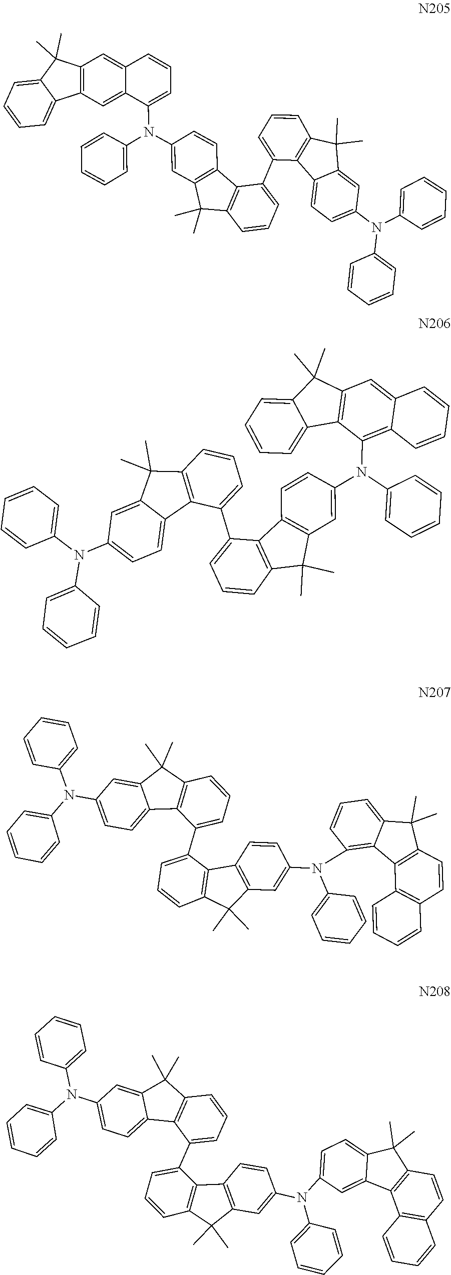 [SMILES: CC1(C)c2ccccc2-c2cc3c(N(c4ccccc4)c4ccc5c(c4)C(C)(C)c4cccc(-c6cccc7c6-c6ccc(N(c8ccccc8)c8ccccc8)cc6C7(C)C)c4-5)cccc3cc21 ; CC1(C)c2cc(N(c3ccccc3)c3ccccc3)ccc2-c2c(-c3cccc4c3-c3ccc(N(c5ccccc5)c5c6c(cc7ccccc57)C(C)(C)c5ccccc5-6)cc3C4(C)C)cccc21 ; CC1(C)c2cc(N(c3ccccc3)c3ccccc3)ccc2-c2c(-c3cccc4c3-c3ccc(N(c5ccccc5)c5cccc6c5-c5c(ccc7ccccc57)C6(C)C)cc3C4(C)C)cccc21 ; CC1(C)c2cc(N(c3ccccc3)c3ccccc3)ccc2-c2c(-c3cccc4c3-c3ccc(N(c5ccccc5)c5ccc6c(c5)-c5c(ccc7ccccc57)C6(C)C)cc3C4(C)C)cccc21]

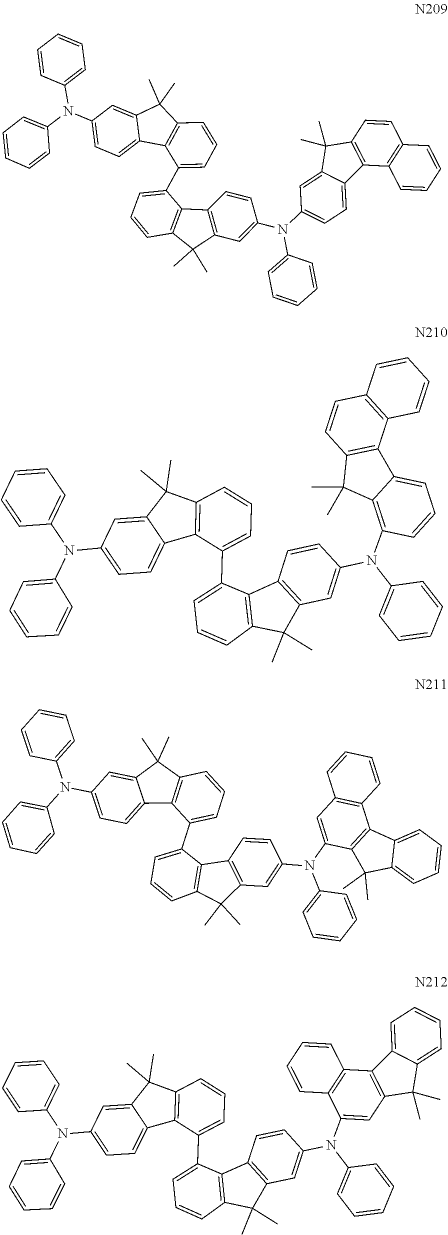 [SMILES: CC1(C)c2cc(N(c3ccccc3)c3ccccc3)ccc2-c2c(-c3cccc4c3-c3ccc(N(c5ccccc5)c5ccc6c(c5)C(C)(C)c5ccc7ccccc7c5-6)cc3C4(C)C)cccc21 ; CC1(C)c2cc(N(c3ccccc3)c3ccccc3)ccc2-c2c(-c3cccc4c3-c3ccc(N(c5ccccc5)c5cccc6c5C(C)(C)c5ccc7ccccc7c5-6)cc3C4(C)C)cccc21 ; CC1(C)c2cc(N(c3ccccc3)c3ccccc3)ccc2-c2c(-c3cccc4c3-c3ccc(N(c5ccccc5)c5cc6ccccc6c6c5C(C)(C)c5ccccc5-6)cc3C4(C)C)cccc21 ; CC1(C)c2cc(N(c3ccccc3)c3ccccc3)ccc2-c2c(-c3cccc4c3-c3ccc(N(c5ccccc5)c5cc6c(c7ccccc57)-c5ccccc5C6(C)C)cc3C4(C)C)cccc21]

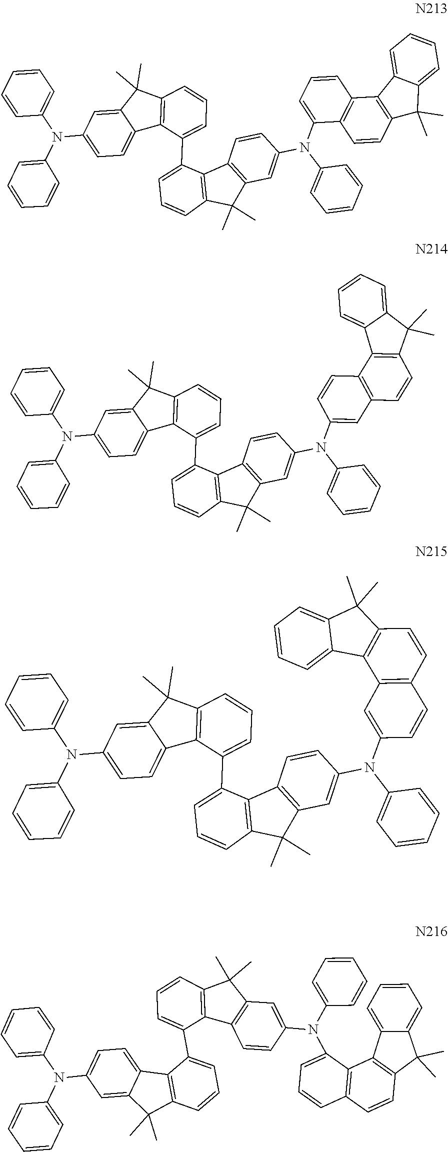 [SMILES: CC1(C)c2cc(N(c3ccccc3)c3ccccc3)ccc2-c2c(-c3cccc4c3-c3ccc(N(c5ccccc5)c5cccc6c7c(ccc56)C(C)(C)c5ccccc5-7)cc3C4(C)C)cccc21 ; CC1(C)c2cc(N(c3ccccc3)c3ccccc3)ccc2-c2c(-c3cccc4c3-c3ccc(N(c5ccccc5)c5ccc6c7c(ccc6c5)C(C)(C)c5ccccc5-7)cc3C4(C)C)cccc21 ; CC1(C)c2cc(N(c3ccccc3)c3ccccc3)ccc2-c2c(-c3cccc4c3-c3ccc(N(c5ccccc5)c5ccc6ccc7c(c6c5)-c5ccccc5C7(C)C)cc3C4(C)C)cccc21 ; CC1(C)c2cc(N(c3ccccc3)c3ccccc3)ccc2-c2c(-c3cccc4c3-c3ccc(N(c5ccccc5)c5cccc6ccc7c(c56)-c5ccccc5C7(C)C)cc3C4(C)C)cccc21]

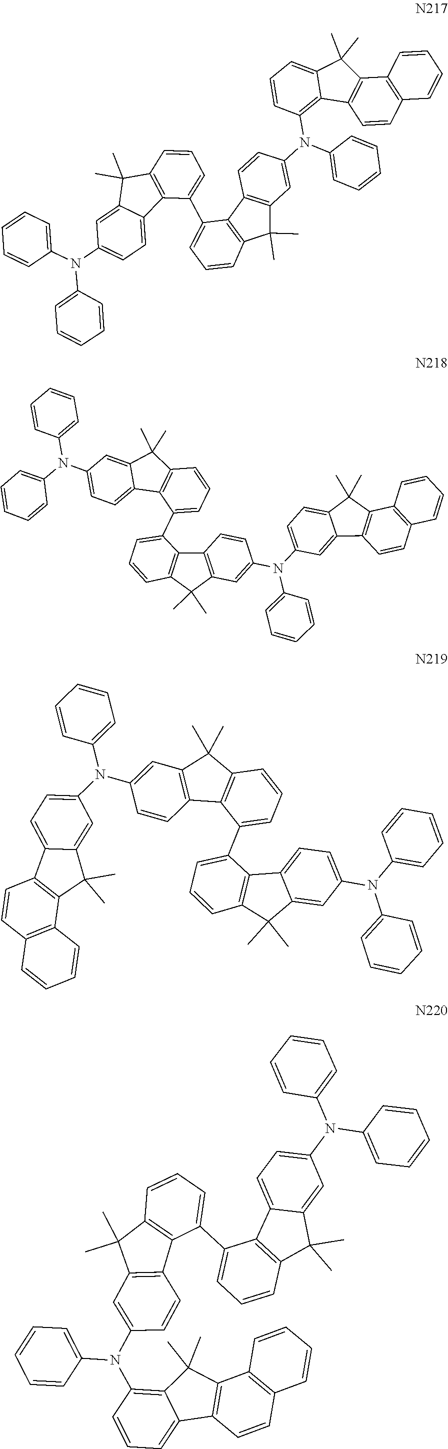 [SMILES: CC1(C)c2cc(N(c3ccccc3)c3ccccc3)ccc2-c2c(-c3cccc4c3-c3ccc(N(c5ccccc5)c5cccc6c5-c5ccc7ccccc7c5C6(C)C)cc3C4(C)C)cccc21 ; CC1(C)c2cc(N(c3ccccc3)c3ccccc3)ccc2-c2c(-c3cccc4c3-c3ccc(N(c5ccccc5)c5ccc6c(c5)-c5ccc7ccccc7c5C6(C)C)cc3C4(C)C)cccc21 ; CC1(C)c2cc(N(c3ccccc3)c3ccccc3)ccc2-c2c(-c3cccc4c3-c3ccc(N(c5ccccc5)c5ccc6c(c5)C(C)(C)c5c-6ccc6ccccc56)cc3C4(C)C)cccc21 ; CC1(C)c2cc(N(c3ccccc3)c3ccccc3)ccc2-c2c(-c3cccc4c3-c3ccc(N(c5ccccc5)c5cccc6c5C(C)(C)c5c-6ccc6ccccc56)cc3C4(C)C)cccc21]

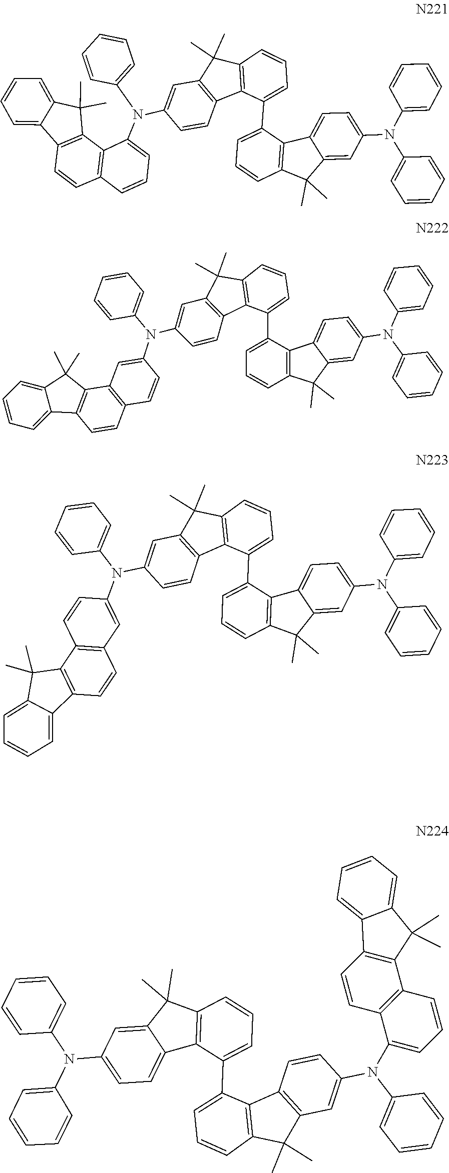 [SMILES: CC1(C)c2cc(N(c3ccccc3)c3ccccc3)ccc2-c2c(-c3cccc4c3-c3ccc(N(c5ccccc5)c5cccc6ccc7c(c56)C(C)(C)c5ccccc5-7)cc3C4(C)C)cccc21 ; CC1(C)c2cc(N(c3ccccc3)c3ccccc3)ccc2-c2c(-c3cccc4c3-c3ccc(N(c5ccccc5)c5ccc6ccc7c(c6c5)C(C)(C)c5ccccc5-7)cc3C4(C)C)cccc21 ; CC1(C)c2cc(N(c3ccccc3)c3ccccc3)ccc2-c2c(-c3cccc4c3-c3ccc(N(c5ccccc5)c5ccc6c7c(ccc6c5)-c5ccccc5C7(C)C)cc3C4(C)C)cccc21 ; CC1(C)c2cc(N(c3ccccc3)c3ccccc3)ccc2-c2c(-c3cccc4c3-c3ccc(N(c5ccccc5)c5cccc6c7c(ccc56)-c5ccccc5C7(C)C)cc3C4(C)C)cccc21]

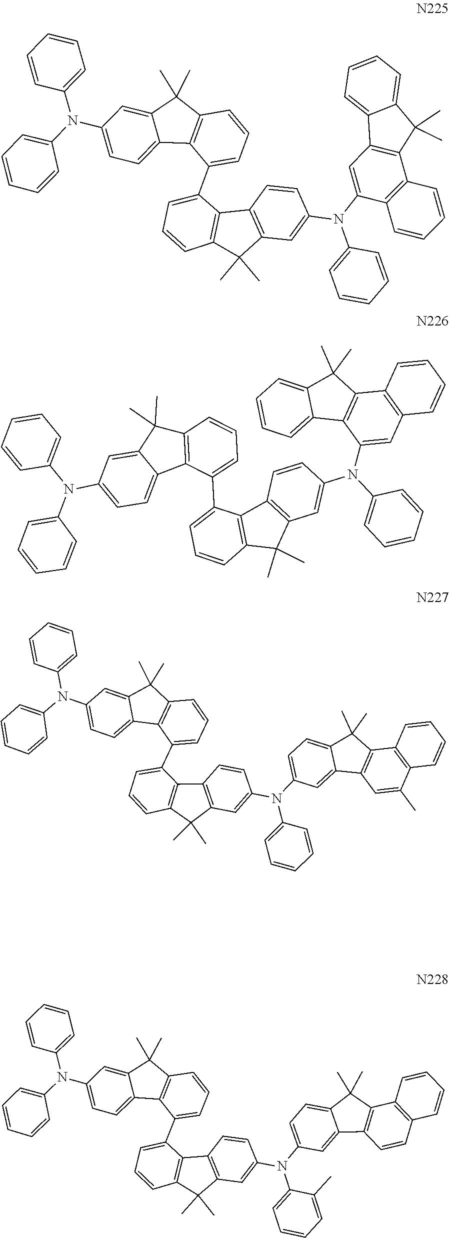 [SMILES: CC1(C)c2cc(N(c3ccccc3)c3ccccc3)ccc2-c2c(-c3cccc4c3-c3ccc(N(c5ccccc5)c5cc6c(c7ccccc57)C(C)(C)c5ccccc5-6)cc3C4(C)C)cccc21 ; CC1(C)c2cc(N(c3ccccc3)c3ccccc3)ccc2-c2c(-c3cccc4c3-c3ccc(N(c5ccccc5)c5cc6ccccc6c6c5-c5ccccc5C6(C)C)cc3C4(C)C)cccc21 ; Cc1cc2c(c3ccccc13)C(C)(C)c1ccc(N(c3ccccc3)c3ccc4c(c3)C(C)(C)c3cccc(-c5cccc6c5-c5ccc(N(c7ccccc7)c7ccccc7)cc5C6(C)C)c3-4)cc1-2 ; Cc1ccccc1N(c1ccc2c(c1)-c1ccc3ccccc3c1C2(C)C)c1ccc2c(c1)C(C)(C)c1cccc(-c3cccc4c3-c3ccc(N(c5ccccc5)c5ccccc5)cc3C4(C)C)c1-2]

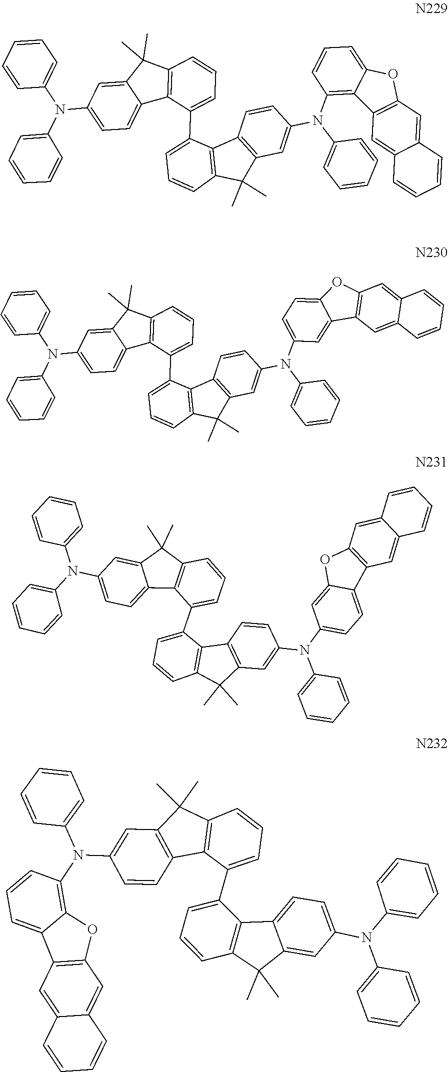 [SMILES: CC1(C)c2cc(N(c3ccccc3)c3ccccc3)ccc2-c2c(-c3cccc4c3-c3ccc(N(c5ccccc5)c5cccc6oc7cc8ccccc8cc7c56)cc3C4(C)C)cccc21 ; CC1(C)c2cc(N(c3ccccc3)c3ccccc3)ccc2-c2c(-c3cccc4c3-c3ccc(N(c5ccccc5)c5ccc6oc7cc8ccccc8cc7c6c5)cc3C4(C)C)cccc21 ; CC1(C)c2cc(N(c3ccccc3)c3ccccc3)ccc2-c2c(-c3cccc4c3-c3ccc(N(c5ccccc5)c5ccc6c(c5)oc5cc7ccccc7cc56)cc3C4(C)C)cccc21 ; CC1(C)c2cc(N(c3ccccc3)c3ccccc3)ccc2-c2c(-c3cccc4c3-c3ccc(N(c5ccccc5)c5cccc6c5oc5cc7ccccc7cc56)cc3C4(C)C)cccc21]

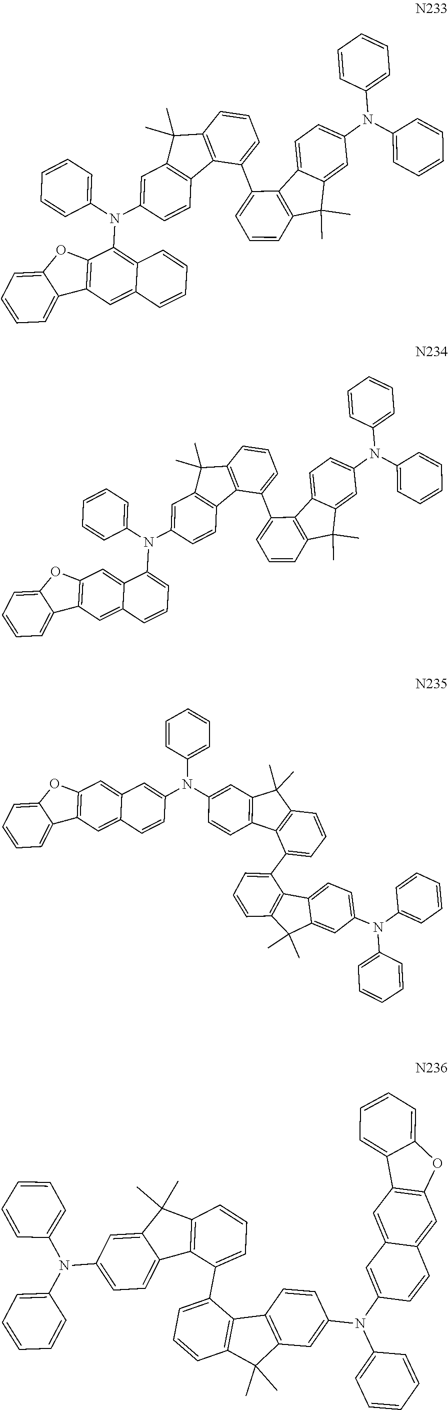 [SMILES: CC1(C)c2cc(N(c3ccccc3)c3ccccc3)ccc2-c2c(-c3cccc4c3-c3ccc(N(c5ccccc5)c5c6ccccc6cc6c5oc5ccccc56)cc3C4(C)C)cccc21 ; CC1(C)c2cc(N(c3ccccc3)c3ccccc3)ccc2-c2c(-c3cccc4c3-c3ccc(N(c5ccccc5)c5cccc6cc7c(cc56)oc5ccccc57)cc3C4(C)C)cccc21 ; CC1(C)c2cc(N(c3ccccc3)c3ccccc3)ccc2-c2c(-c3cccc4c3-c3ccc(N(c5ccccc5)c5ccc6cc7c(cc6c5)oc5ccccc57)cc3C4(C)C)cccc21 ; CC1(C)c2cc(N(c3ccccc3)c3ccccc3)ccc2-c2c(-c3cccc4c3-c3ccc(N(c5ccccc5)c5ccc6cc7oc8ccccc8c7cc6c5)cc3C4(C)C)cccc21]

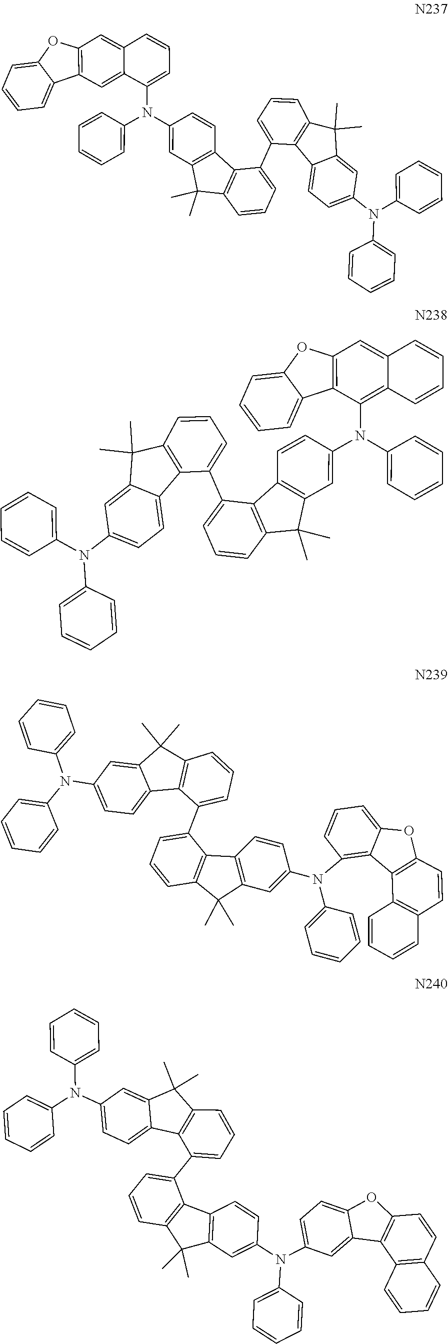 [SMILES: CC1(C)c2cc(N(c3ccccc3)c3ccccc3)ccc2-c2c(-c3cccc4c3-c3ccc(N(c5ccccc5)c5cccc6cc7oc8ccccc8c7cc56)cc3C4(C)C)cccc21 ; CC1(C)c2cc(N(c3ccccc3)c3ccccc3)ccc2-c2c(-c3cccc4c3-c3ccc(N(c5ccccc5)c5c6ccccc6cc6oc7ccccc7c56)cc3C4(C)C)cccc21 ; CC1(C)c2cc(N(c3ccccc3)c3ccccc3)ccc2-c2c(-c3cccc4c3-c3ccc(N(c5ccccc5)c5cccc6oc7ccc8ccccc8c7c56)cc3C4(C)C)cccc21 ; CC1(C)c2cc(N(c3ccccc3)c3ccccc3)ccc2-c2c(-c3cccc4c3-c3ccc(N(c5ccccc5)c5ccc6oc7ccc8ccccc8c7c6c5)cc3C4(C)C)cccc21]

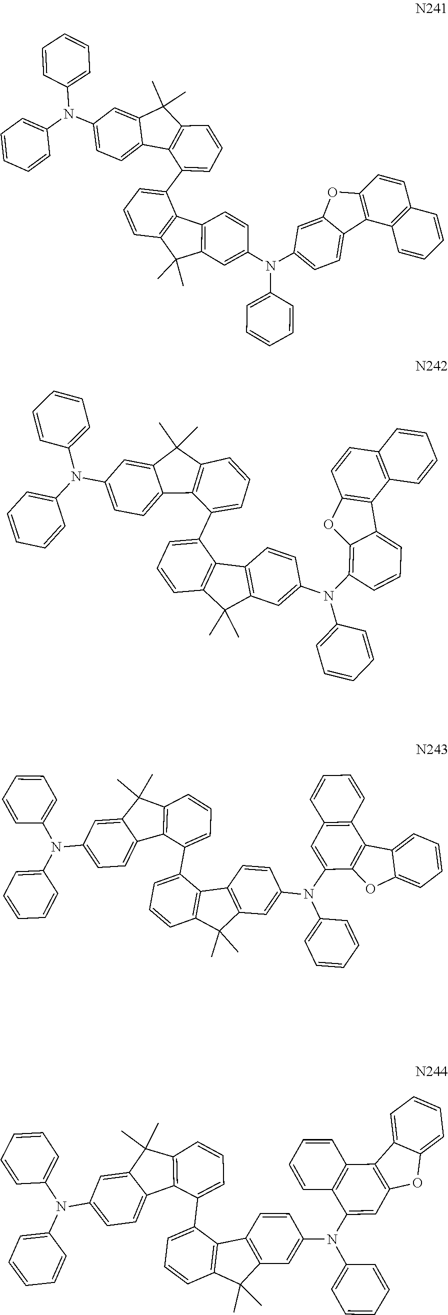 [SMILES: CC1(C)c2cc(N(c3ccccc3)c3ccccc3)ccc2-c2c(-c3cccc4c3-c3ccc(N(c5ccccc5)c5ccc6c(c5)oc5ccc7ccccc7c56)cc3C4(C)C)cccc21 ; CC1(C)c2cc(N(c3ccccc3)c3ccccc3)ccc2-c2c(-c3cccc4c3-c3ccc(N(c5ccccc5)c5cccc6c5oc5ccc7ccccc7c56)cc3C4(C)C)cccc21 ; CC1(C)c2cc(N(c3ccccc3)c3ccccc3)ccc2-c2c(-c3cccc4c3-c3ccc(N(c5ccccc5)c5cc6ccccc6c6c5oc5ccccc56)cc3C4(C)C)cccc21 ; CC1(C)c2cc(N(c3ccccc3)c3ccccc3)ccc2-c2c(-c3cccc4c3-c3ccc(N(c5ccccc5)c5cc6oc7ccccc7c6c6ccccc56)cc3C4(C)C)cccc21]

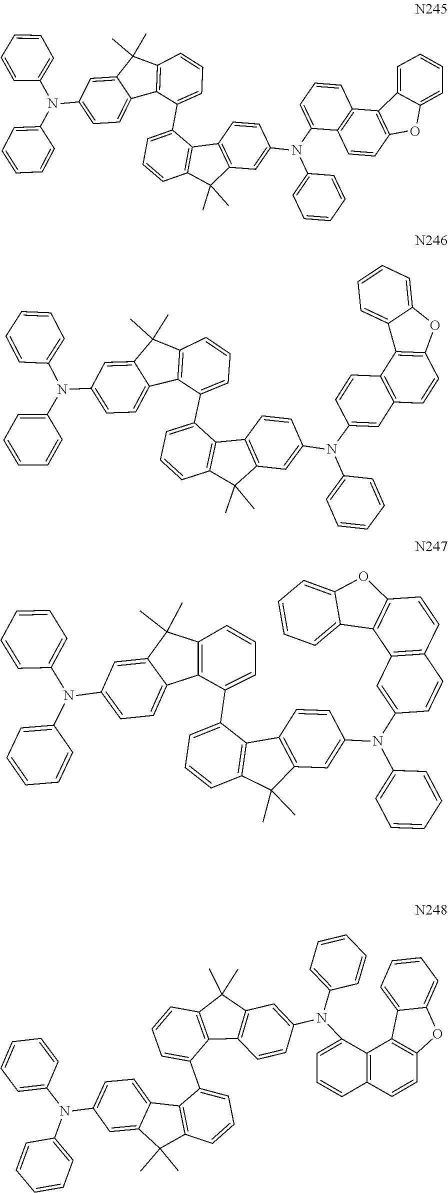 [SMILES: CC1(C)c2cc(N(c3ccccc3)c3ccccc3)ccc2-c2c(-c3cccc4c3-c3ccc(N(c5ccccc5)c5cccc6c5ccc5oc7ccccc7c56)cc3C4(C)C)cccc21 ; CC1(C)c2cc(N(c3ccccc3)c3ccccc3)ccc2-c2c(-c3cccc4c3-c3ccc(N(c5ccccc5)c5ccc6c(ccc7oc8ccccc8c76)c5)cc3C4(C)C)cccc21 ; CC1(C)c2cc(N(c3ccccc3)c3ccccc3)ccc2-c2c(-c3cccc4c3-c3ccc(N(c5ccccc5)c5ccc6ccc7oc8ccccc8c7c6c5)cc3C4(C)C)cccc21 ; CC1(C)c2cc(N(c3ccccc3)c3ccccc3)ccc2-c2c(-c3cccc4c3-c3ccc(N(c5ccccc5)c5cccc6ccc7oc8ccccc8c7c56)cc3C4(C)C)cccc21]

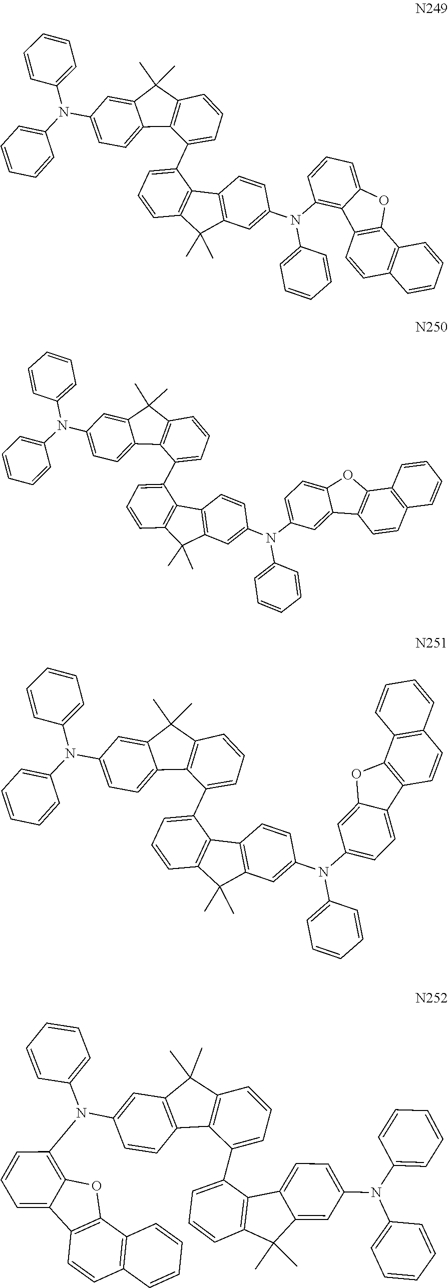 [SMILES: CC1(C)c2cc(N(c3ccccc3)c3ccccc3)ccc2-c2c(-c3cccc4c3-c3ccc(N(c5ccccc5)c5cccc6oc7c8ccccc8ccc7c56)cc3C4(C)C)cccc21 ; CC1(C)c2cc(N(c3ccccc3)c3ccccc3)ccc2-c2c(-c3cccc4c3-c3ccc(N(c5ccccc5)c5ccc6oc7c8ccccc8ccc7c6c5)cc3C4(C)C)cccc21 ; CC1(C)c2cc(N(c3ccccc3)c3ccccc3)ccc2-c2c(-c3cccc4c3-c3ccc(N(c5ccccc5)c5ccc6c(c5)oc5c7ccccc7ccc65)cc3C4(C)C)cccc21 ; CC1(C)c2cc(N(c3ccccc3)c3ccccc3)ccc2-c2c(-c3cccc4c3-c3ccc(N(c5ccccc5)c5cccc6c5oc5c7ccccc7ccc65)cc3C4(C)C)cccc21]

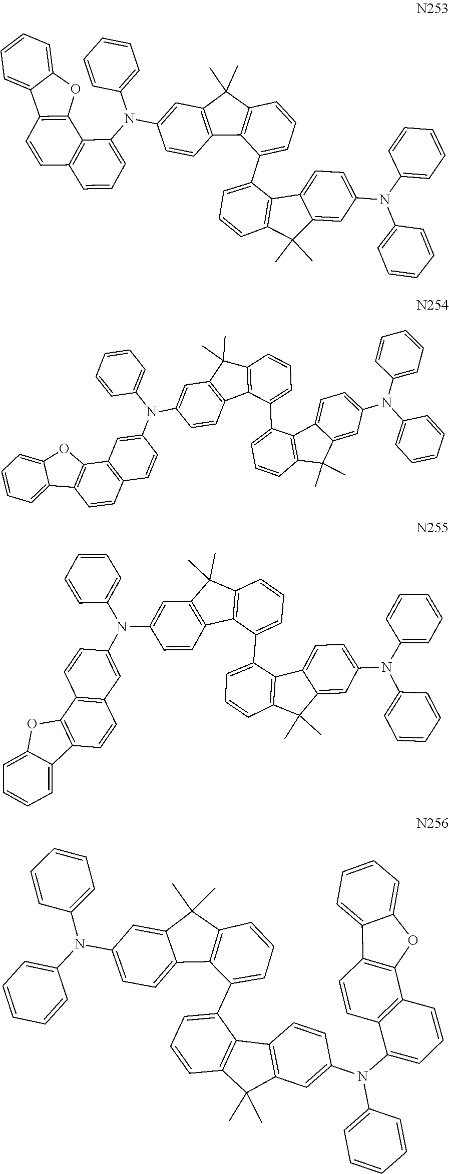 [SMILES: CC1(C)c2cc(N(c3ccccc3)c3ccccc3)ccc2-c2c(-c3cccc4c3-c3ccc(N(c5ccccc5)c5cccc6ccc7c8ccccc8oc7c56)cc3C4(C)C)cccc21 ; CC1(C)c2cc(N(c3ccccc3)c3ccccc3)ccc2-c2c(-c3cccc4c3-c3ccc(N(c5ccccc5)c5ccc6ccc7c8ccccc8oc7c6c5)cc3C4(C)C)cccc21 ; CC1(C)c2cc(N(c3ccccc3)c3ccccc3)ccc2-c2c(-c3cccc4c3-c3ccc(N(c5ccccc5)c5ccc6c(ccc7c8ccccc8oc67)c5)cc3C4(C)C)cccc21 ; CC1(C)c2cc(N(c3ccccc3)c3ccccc3)ccc2-c2c(-c3cccc4c3-c3ccc(N(c5ccccc5)c5cccc6c5ccc5c7ccccc7oc65)cc3C4(C)C)cccc21]

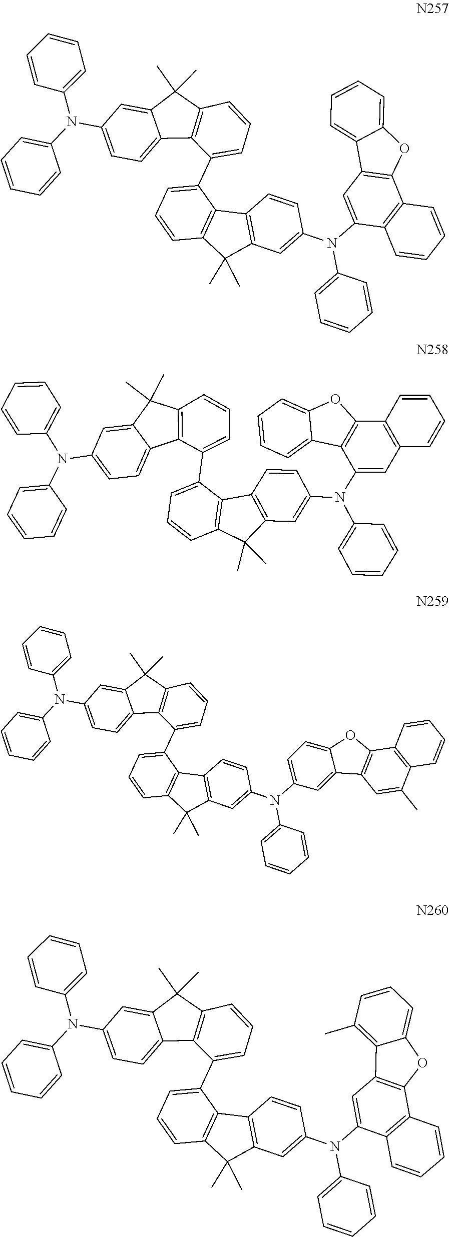 [SMILES: CC1(C)c2cc(N(c3ccccc3)c3ccccc3)ccc2-c2c(-c3cccc4c3-c3ccc(N(c5ccccc5)c5cc6c7ccccc7oc6c6ccccc56)cc3C4(C)C)cccc21 ; CC1(C)c2cc(N(c3ccccc3)c3ccccc3)ccc2-c2c(-c3cccc4c3-c3ccc(N(c5ccccc5)c5cc6ccccc6c6oc7ccccc7c56)cc3C4(C)C)cccc21 ; Cc1cc2c3cc(N(c4ccccc4)c4ccc5c(c4)C(C)(C)c4cccc(-c6cccc7c6-c6ccc(N(c8ccccc8)c8ccccc8)cc6C7(C)C)c4-5)ccc3oc2c2ccccc12 ; Cc1cccc2oc3c4ccccc4c(N(c4ccccc4)c4ccc5c(c4)C(C)(C)c4cccc(-c6cccc7c6-c6ccc(N(c8ccccc8)c8ccccc8)cc6C7(C)C)c4-5)cc3c12]

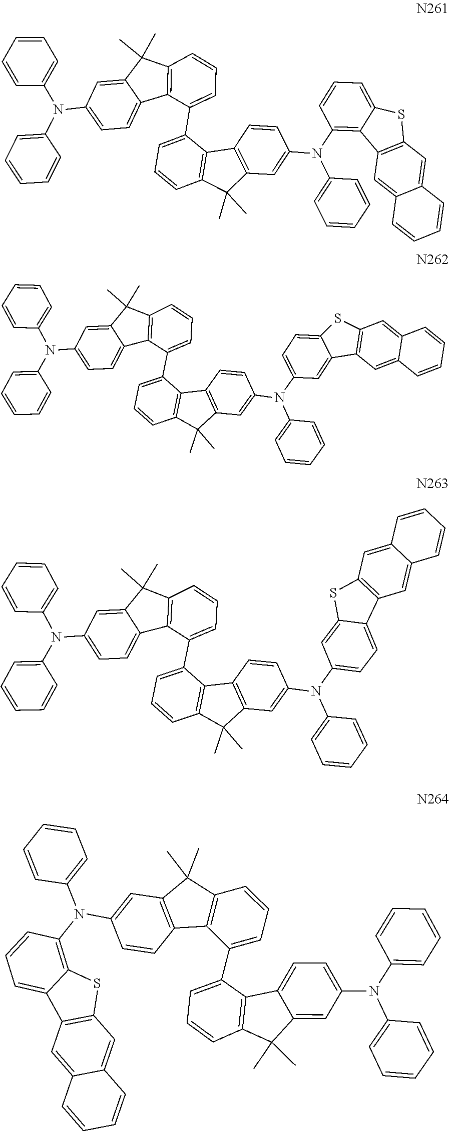 [SMILES: CC1(C)c2cc(N(c3ccccc3)c3ccccc3)ccc2-c2c(-c3cccc4c3-c3ccc(N(c5ccccc5)c5cccc6sc7cc8ccccc8cc7c56)cc3C4(C)C)cccc21 ; CC1(C)c2cc(N(c3ccccc3)c3ccccc3)ccc2-c2c(-c3cccc4c3-c3ccc(N(c5ccccc5)c5ccc6sc7cc8ccccc8cc7c6c5)cc3C4(C)C)cccc21 ; CC1(C)c2cc(N(c3ccccc3)c3ccccc3)ccc2-c2c(-c3cccc4c3-c3ccc(N(c5ccccc5)c5ccc6c(c5)sc5cc7ccccc7cc56)cc3C4(C)C)cccc21 ; CC1(C)c2cc(N(c3ccccc3)c3ccccc3)ccc2-c2c(-c3cccc4c3-c3ccc(N(c5ccccc5)c5cccc6c5sc5cc7ccccc7cc56)cc3C4(C)C)cccc21]

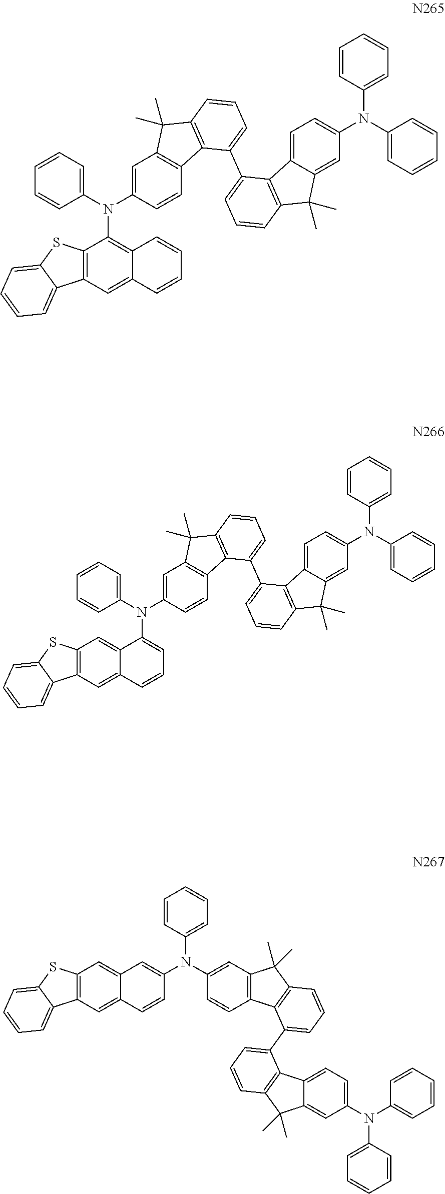 [SMILES: CC1(C)c2cc(N(c3ccccc3)c3ccccc3)ccc2-c2c(-c3cccc4c3-c3ccc(N(c5ccccc5)c5c6ccccc6cc6c5sc5ccccc56)cc3C4(C)C)cccc21 ; CC1(C)c2cc(N(c3ccccc3)c3ccccc3)ccc2-c2c(-c3cccc4c3-c3ccc(N(c5ccccc5)c5cccc6cc7c(cc56)sc5ccccc57)cc3C4(C)C)cccc21 ; CC1(C)c2cc(N(c3ccccc3)c3ccccc3)ccc2-c2c(-c3cccc4c3-c3ccc(N(c5ccccc5)c5ccc6cc7c(cc6c5)sc5ccccc57)cc3C4(C)C)cccc21]

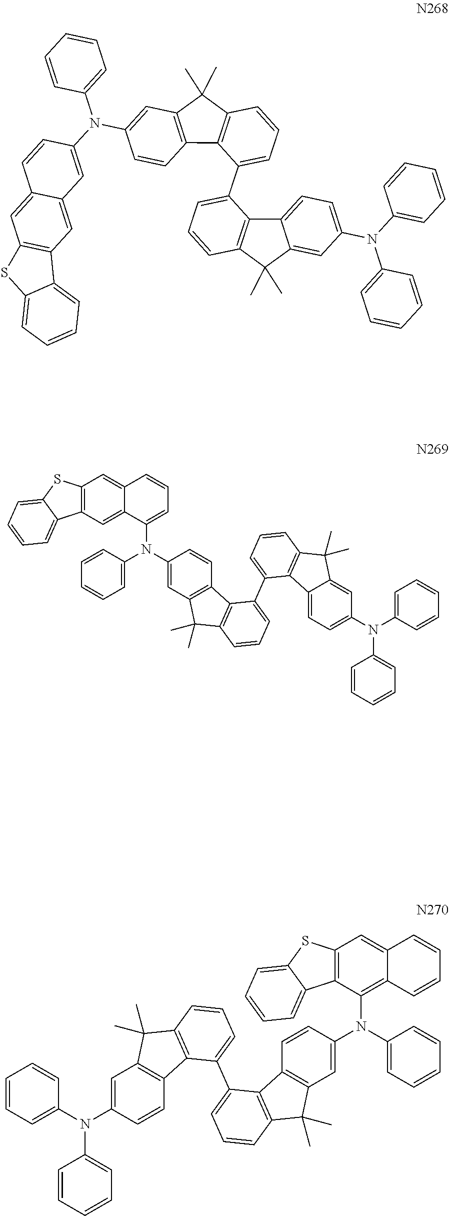 [SMILES: CC1(C)c2cc(N(c3ccccc3)c3ccccc3)ccc2-c2c(-c3cccc4c3-c3ccc(N(c5ccccc5)c5ccc6cc7sc8ccccc8c7cc6c5)cc3C4(C)C)cccc21 ; CC1(C)c2cc(N(c3ccccc3)c3ccccc3)ccc2-c2c(-c3cccc4c3-c3ccc(N(c5ccccc5)c5cccc6cc7sc8ccccc8c7cc56)cc3C4(C)C)cccc21 ; CC1(C)c2cc(N(c3ccccc3)c3ccccc3)ccc2-c2c(-c3cccc4c3-c3ccc(N(c5ccccc5)c5c6ccccc6cc6sc7ccccc7c56)cc3C4(C)C)cccc21]

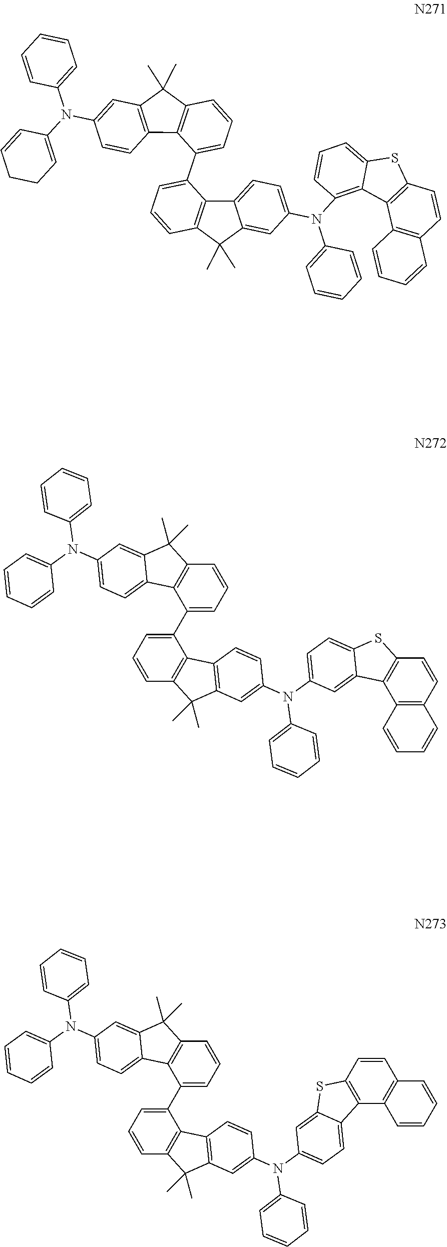 [SMILES: CC1(C)c2cc(N(C3=CCCC=C3)c3ccccc3)ccc2-c2c(-c3cccc4c3-c3ccc(N(c5ccccc5)c5cccc6sc7ccc8ccccc8c7c56)cc3C4(C)C)cccc21 ; CC1(C)c2cc(N(c3ccccc3)c3ccccc3)ccc2-c2c(-c3cccc4c3-c3ccc(N(c5ccccc5)c5ccc6sc7ccc8ccccc8c7c6c5)cc3C4(C)C)cccc21 ; CC1(C)c2cc(N(c3ccccc3)c3ccccc3)ccc2-c2c(-c3cccc4c3-c3ccc(N(c5ccccc5)c5ccc6c(c5)sc5ccc7ccccc7c56)cc3C4(C)C)cccc21]

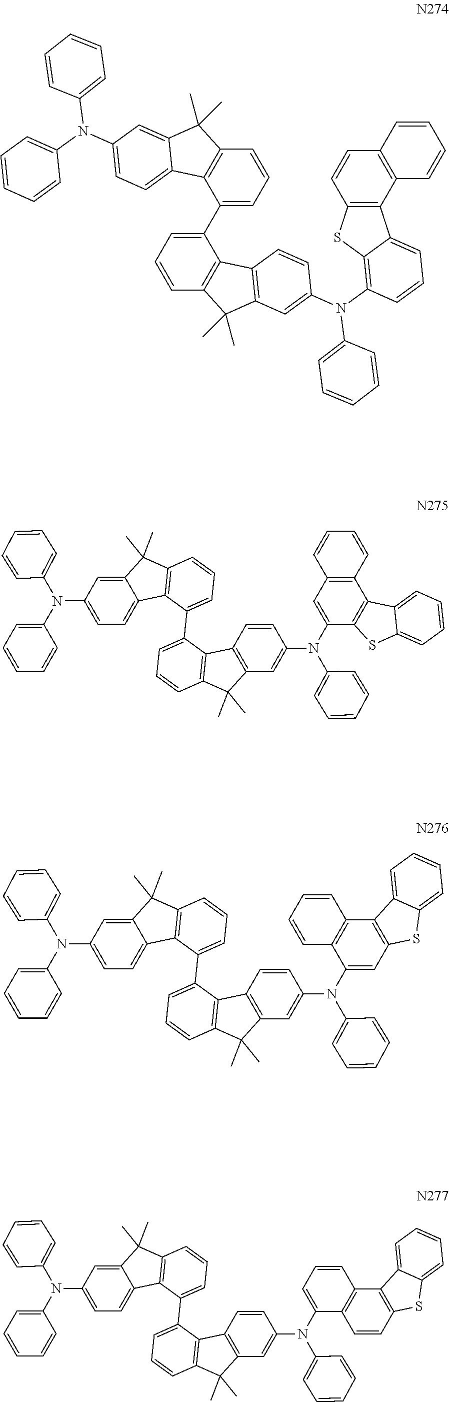 [SMILES: CC1(C)c2cc(N(c3ccccc3)c3ccccc3)ccc2-c2c(-c3cccc4c3-c3ccc(N(c5ccccc5)c5cccc6c5sc5ccc7ccccc7c56)cc3C4(C)C)cccc21 ; CC1(C)c2cc(N(c3ccccc3)c3ccccc3)ccc2-c2c(-c3cccc4c3-c3ccc(N(c5ccccc5)c5cc6ccccc6c6c5sc5ccccc56)cc3C4(C)C)cccc21 ; CC1(C)c2cc(N(c3ccccc3)c3ccccc3)ccc2-c2c(-c3cccc4c3-c3ccc(N(c5ccccc5)c5cc6sc7ccccc7c6c6ccccc56)cc3C4(C)C)cccc21 ; CC1(C)c2cc(N(c3ccccc3)c3ccccc3)ccc2-c2c(-c3cccc4c3-c3ccc(N(c5ccccc5)c5cccc6c5ccc5sc7ccccc7c56)cc3C4(C)C)cccc21]

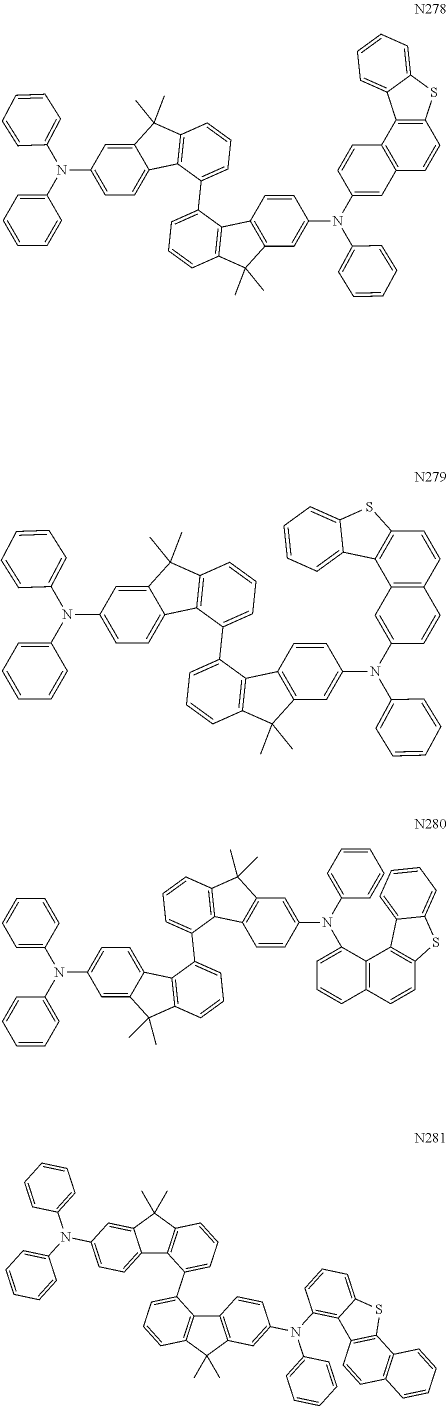 [SMILES: CC1(C)c2cc(N(c3ccccc3)c3ccccc3)ccc2-c2c(-c3cccc4c3-c3ccc(N(c5ccccc5)c5ccc6c(ccc7sc8ccccc8c76)c5)cc3C4(C)C)cccc21 ; CC1(C)c2cc(N(c3ccccc3)c3ccccc3)ccc2-c2c(-c3cccc4c3-c3ccc(N(c5ccccc5)c5ccc6ccc7sc8ccccc8c7c6c5)cc3C4(C)C)cccc21 ; CC1(C)c2cc(N(c3ccccc3)c3ccccc3)ccc2-c2c(-c3cccc4c3-c3ccc(N(c5ccccc5)c5cccc6ccc7sc8ccccc8c7c56)cc3C4(C)C)cccc21 ; CC1(C)c2cc(N(c3ccccc3)c3ccccc3)ccc2-c2c(-c3cccc4c3-c3ccc(N(c5ccccc5)c5cccc6sc7c8ccccc8ccc7c56)cc3C4(C)C)cccc21]

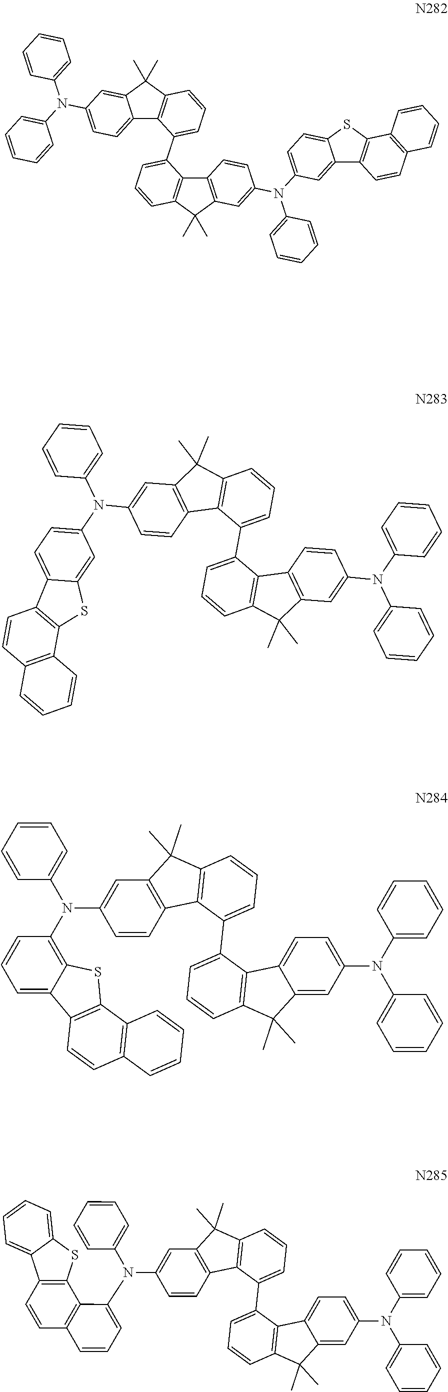 [SMILES: CC1(C)c2cc(N(c3ccccc3)c3ccccc3)ccc2-c2c(-c3cccc4c3-c3ccc(N(c5ccccc5)c5ccc6sc7c8ccccc8ccc7c6c5)cc3C4(C)C)cccc21 ; CC1(C)c2cc(N(c3ccccc3)c3ccccc3)ccc2-c2c(-c3cccc4c3-c3ccc(N(c5ccccc5)c5ccc6c(c5)sc5c7ccccc7ccc65)cc3C4(C)C)cccc21 ; CC1(C)c2cc(N(c3ccccc3)c3ccccc3)ccc2-c2c(-c3cccc4c3-c3ccc(N(c5ccccc5)c5cccc6c5sc5c7ccccc7ccc65)cc3C4(C)C)cccc21 ; CC1(C)c2cc(N(c3ccccc3)c3ccccc3)ccc2-c2c(-c3cccc4c3-c3ccc(N(c5ccccc5)c5cccc6ccc7c8ccccc8sc7c56)cc3C4(C)C)cccc21]

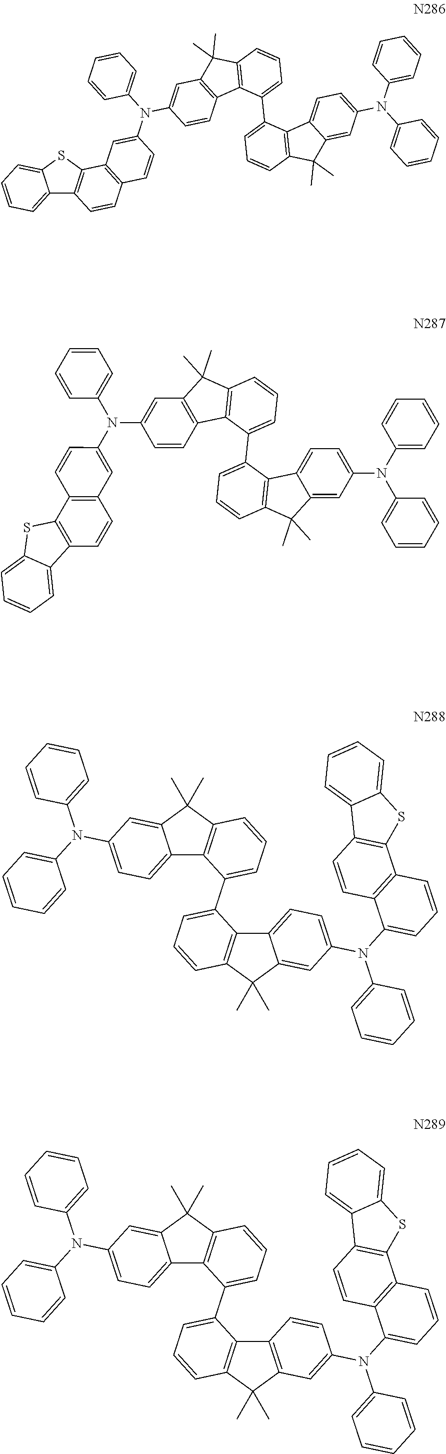 [SMILES: CC1(C)c2cc(N(c3ccccc3)c3ccccc3)ccc2-c2c(-c3cccc4c3-c3ccc(N(c5ccccc5)c5ccc6ccc7c8ccccc8sc7c6c5)cc3C4(C)C)cccc21 ; CC1(C)c2cc(N(c3ccccc3)c3ccccc3)ccc2-c2c(-c3cccc4c3-c3ccc(N(c5ccccc5)c5ccc6c(ccc7c8ccccc8sc67)c5)cc3C4(C)C)cccc21 ; CC1(C)c2cc(N(c3ccccc3)c3ccccc3)ccc2-c2c(-c3cccc4c3-c3ccc(N(c5ccccc5)c5cccc6c5ccc5c7ccccc7sc65)cc3C4(C)C)cccc21 ; CC1(C)c2cc(N(c3ccccc3)c3ccccc3)ccc2-c2c(-c3cccc4c3-c3ccc(N(c5ccccc5)c5cccc6c5ccc5c7ccccc7sc65)cc3C4(C)C)cccc21]

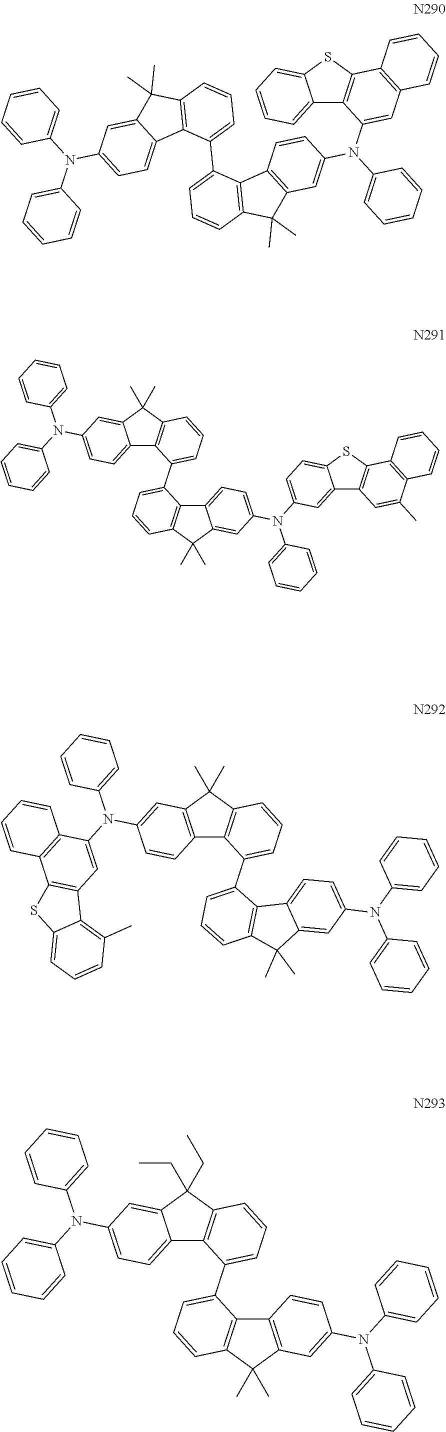 [SMILES: CC1(C)c2cc(N(c3ccccc3)c3ccccc3)ccc2-c2c(-c3cccc4c3-c3ccc(N(c5ccccc5)c5cc6ccccc6c6sc7ccccc7c56)cc3C4(C)C)cccc21 ; Cc1cc2c3cc(N(c4ccccc4)c4ccc5c(c4)C(C)(C)c4cccc(-c6cccc7c6-c6ccc(N(c8ccccc8)c8ccccc8)cc6C7(C)C)c4-5)ccc3sc2c2ccccc12 ; Cc1cccc2sc3c4ccccc4c(N(c4ccccc4)c4ccc5c(c4)C(C)(C)c4cccc(-c6cccc7c6-c6ccc(N(c8ccccc8)c8ccccc8)cc6C7(C)C)c4-5)cc3c12 ; CCC1(CC)c2cc(N(c3ccccc3)c3ccccc3)ccc2-c2c(-c3cccc4c3-c3ccc(N(c5ccccc5)c5ccccc5)cc3C4(C)C)cccc21]

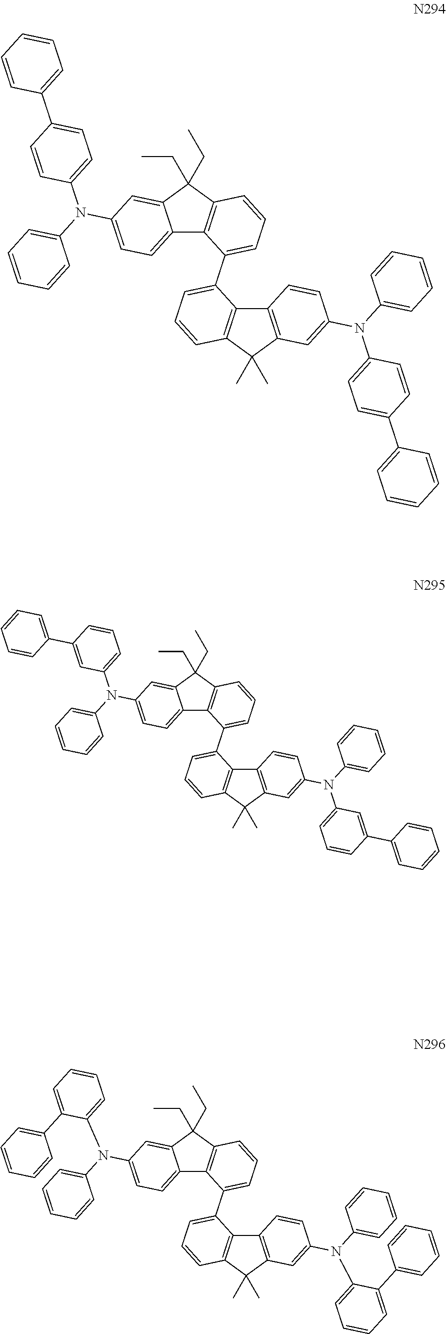 [SMILES: CCC1(CC)c2cc(N(c3ccccc3)c3ccc(-c4ccccc4)cc3)ccc2-c2c(-c3cccc4c3-c3ccc(N(c5ccccc5)c5ccc(-c6ccccc6)cc5)cc3C4(C)C)cccc21 ; CCC1(CC)c2cc(N(c3ccccc3)c3cccc(-c4ccccc4)c3)ccc2-c2c(-c3cccc4c3-c3ccc(N(c5ccccc5)c5cccc(-c6ccccc6)c5)cc3C4(C)C)cccc21 ; CCC1(CC)c2cc(N(c3ccccc3)c3ccccc3-c3ccccc3)ccc2-c2c(-c3cccc4c3-c3ccc(N(c5ccccc5)c5ccccc5-c5ccccc5)cc3C4(C)C)cccc21]

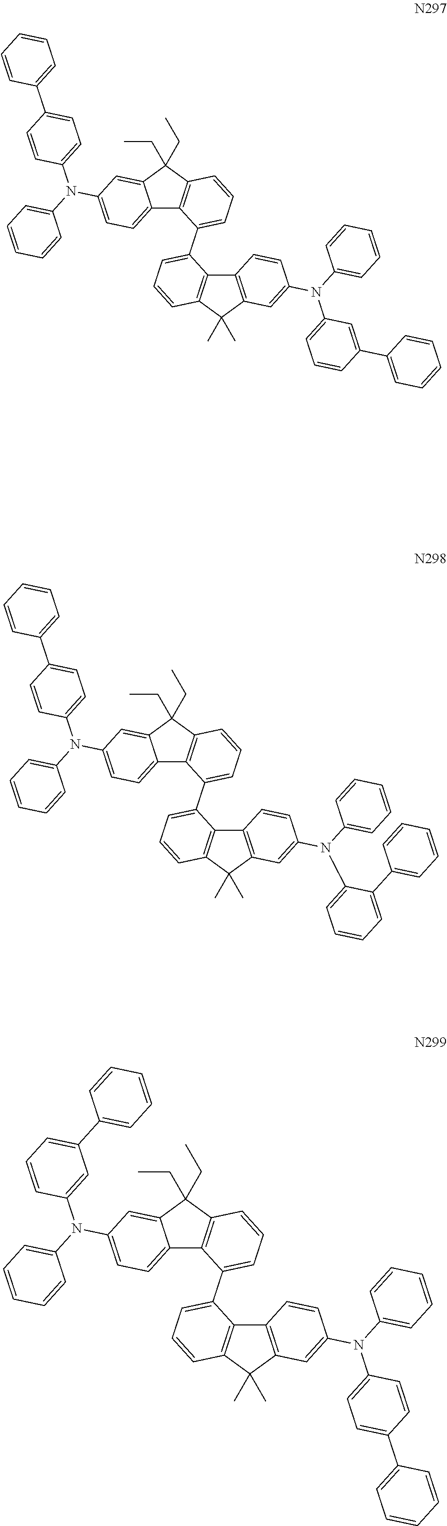 [SMILES: CCC1(CC)c2cc(N(c3ccccc3)c3ccc(-c4ccccc4)cc3)ccc2-c2c(-c3cccc4c3-c3ccc(N(c5ccccc5)c5cccc(-c6ccccc6)c5)cc3C4(C)C)cccc21 ; CCC1(CC)c2cc(N(c3ccccc3)c3ccc(-c4ccccc4)cc3)ccc2-c2c(-c3cccc4c3-c3ccc(N(c5ccccc5)c5ccccc5-c5ccccc5)cc3C4(C)C)cccc21 ; CCC1(CC)c2cc(N(c3ccccc3)c3cccc(-c4ccccc4)c3)ccc2-c2c(-c3cccc4c3-c3ccc(N(c5ccccc5)c5ccc(-c6ccccc6)cc5)cc3C4(C)C)cccc21]

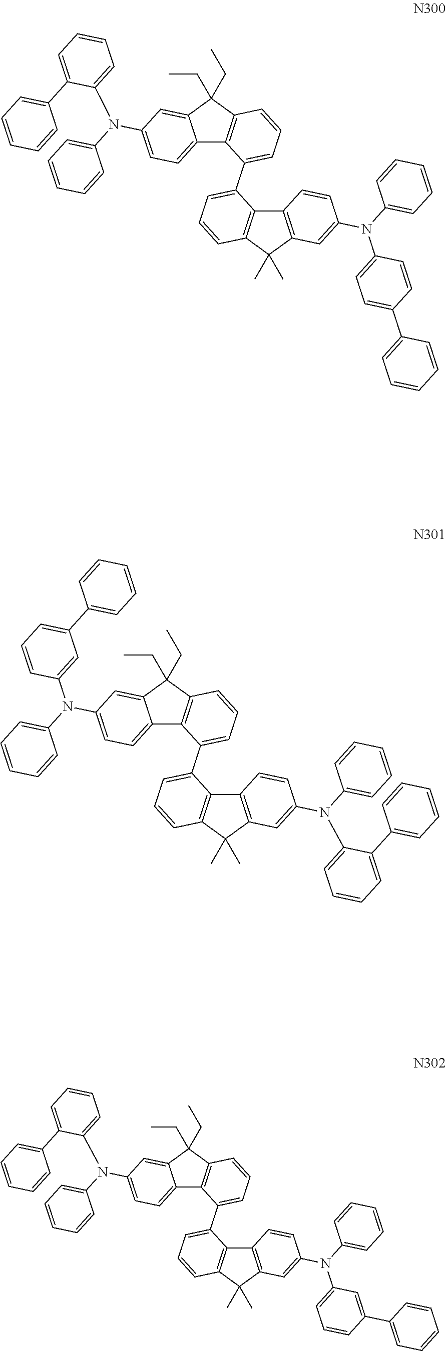 [SMILES: CCC1(CC)c2cc(N(c3ccccc3)c3ccccc3-c3ccccc3)ccc2-c2c(-c3cccc4c3-c3ccc(N(c5ccccc5)c5ccc(-c6ccccc6)cc5)cc3C4(C)C)cccc21 ; CCC1(CC)c2cc(N(c3ccccc3)c3cccc(-c4ccccc4)c3)ccc2-c2c(-c3cccc4c3-c3ccc(N(c5ccccc5)c5ccccc5-c5ccccc5)cc3C4(C)C)cccc21 ; CCC1(CC)c2cc(N(c3ccccc3)c3ccccc3-c3ccccc3)ccc2-c2c(-c3cccc4c3-c3ccc(N(c5ccccc5)c5cccc(-c6ccccc6)c5)cc3C4(C)C)cccc21]

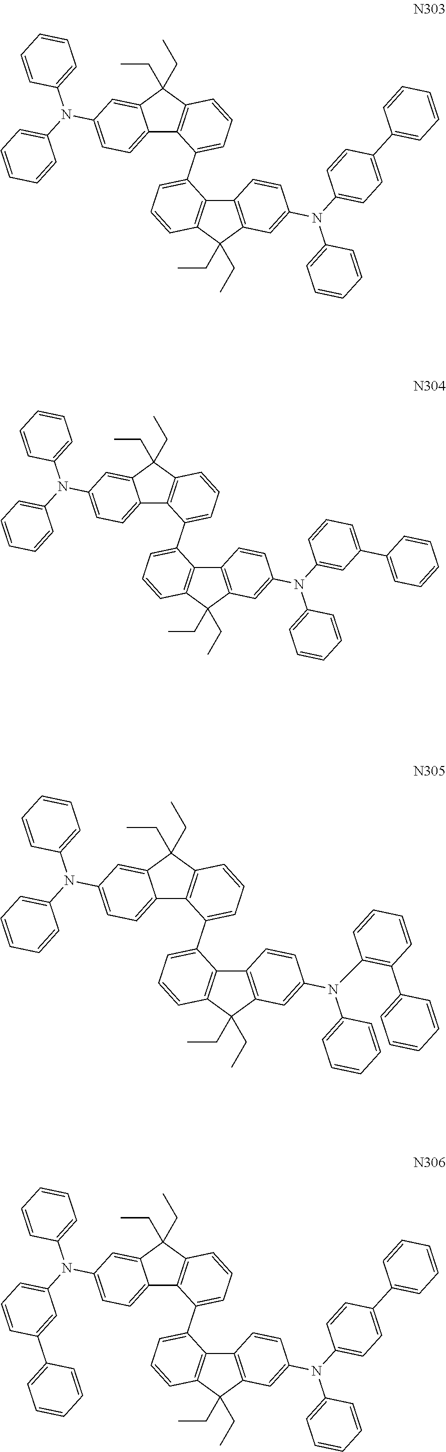 [SMILES: CCC1(CC)c2cc(N(c3ccccc3)c3ccccc3)ccc2-c2c(-c3cccc4c3-c3ccc(N(c5ccccc5)c5ccc(-c6ccccc6)cc5)cc3C4(CC)CC)cccc21 ; CCC1(CC)c2cc(N(c3ccccc3)c3ccccc3)ccc2-c2c(-c3cccc4c3-c3ccc(N(c5ccccc5)c5cccc(-c6ccccc6)c5)cc3C4(CC)CC)cccc21 ; CCC1(CC)c2cc(N(c3ccccc3)c3ccccc3)ccc2-c2c(-c3cccc4c3-c3ccc(N(c5ccccc5)c5ccccc5-c5ccccc5)cc3C4(CC)CC)cccc21 ; CCC1(CC)c2cc(N(c3ccccc3)c3ccc(-c4ccccc4)cc3)ccc2-c2c(-c3cccc4c3-c3ccc(N(c5ccccc5)c5cccc(-c6ccccc6)c5)cc3C4(CC)CC)cccc21]

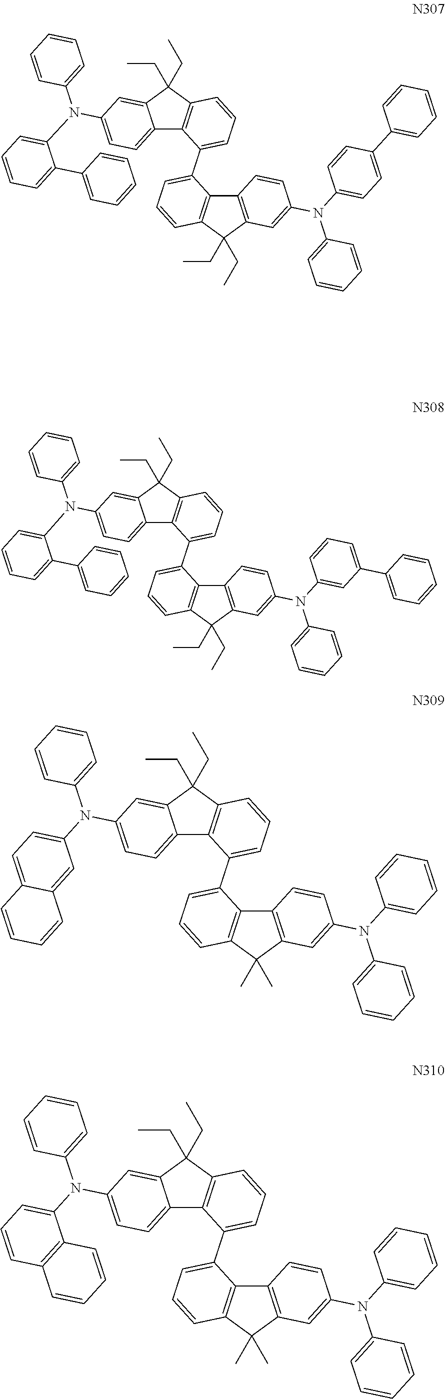 [SMILES: CCC1(CC)c2cc(N(c3ccccc3)c3ccc(-c4ccccc4)cc3)ccc2-c2c(-c3cccc4c3-c3ccc(N(c5ccccc5)c5ccccc5-c5ccccc5)cc3C4(CC)CC)cccc21 ; CCC1(CC)c2cc(N(c3ccccc3)c3cccc(-c4ccccc4)c3)ccc2-c2c(-c3cccc4c3-c3ccc(N(c5ccccc5)c5ccccc5-c5ccccc5)cc3C4(CC)CC)cccc21 ; CCC1(CC)c2cc(N(c3ccccc3)c3ccc4ccccc4c3)ccc2-c2c(-c3cccc4c3-c3ccc(N(c5ccccc5)c5ccccc5)cc3C4(C)C)cccc21 ; CCC1(CC)c2cc(N(c3ccccc3)c3cccc4ccccc34)ccc2-c2c(-c3cccc4c3-c3ccc(N(c5ccccc5)c5ccccc5)cc3C4(C)C)cccc21]

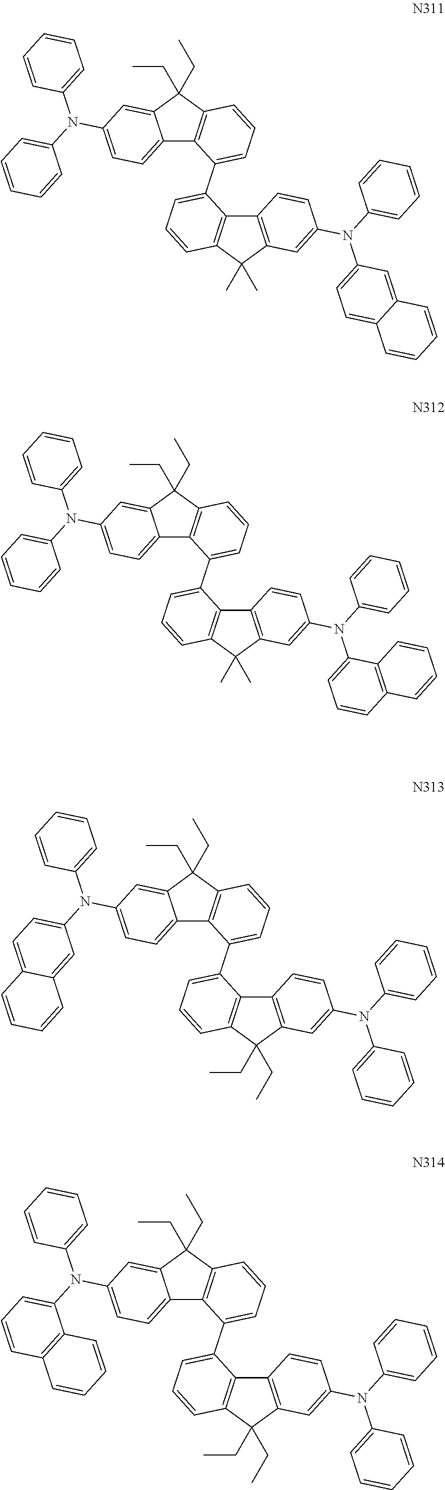 [SMILES: CCC1(CC)c2cc(N(c3ccccc3)c3ccccc3)ccc2-c2c(-c3cccc4c3-c3ccc(N(c5ccccc5)c5ccc6ccccc6c5)cc3C4(C)C)cccc21 ; CCC1(CC)c2cc(N(c3ccccc3)c3ccccc3)ccc2-c2c(-c3cccc4c3-c3ccc(N(c5ccccc5)c5cccc6ccccc56)cc3C4(C)C)cccc21 ; CCC1(CC)c2cc(N(c3ccccc3)c3ccccc3)ccc2-c2c(-c3cccc4c3-c3ccc(N(c5ccccc5)c5ccc6ccccc6c5)cc3C4(CC)CC)cccc21 ; CCC1(CC)c2cc(N(c3ccccc3)c3ccccc3)ccc2-c2c(-c3cccc4c3-c3ccc(N(c5ccccc5)c5cccc6ccccc56)cc3C4(CC)CC)cccc21]

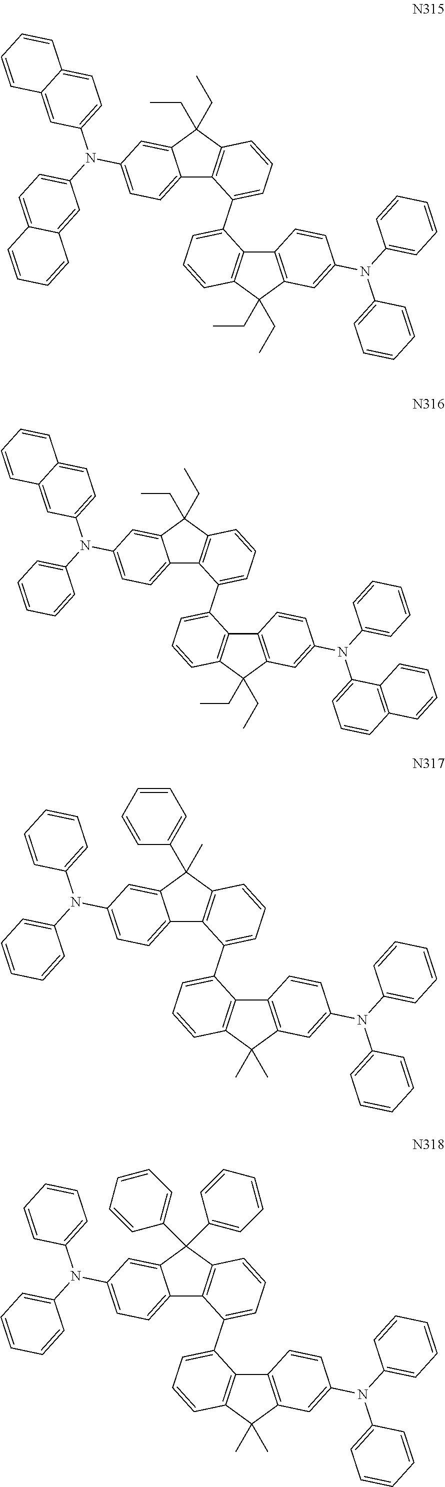 [SMILES: CCC1(CC)c2cc(N(c3ccccc3)c3ccccc3)ccc2-c2c(-c3cccc4c3-c3ccc(N(c5ccc6ccccc6c5)c5ccc6ccccc6c5)cc3C4(CC)CC)cccc21 ; CCC1(CC)c2cc(N(c3ccccc3)c3ccc4ccccc4c3)ccc2-c2c(-c3cccc4c3-c3ccc(N(c5ccccc5)c5cccc6ccccc56)cc3C4(CC)CC)cccc21 ; CC1(C)c2cc(N(c3ccccc3)c3ccccc3)ccc2-c2c(-c3cccc4c3-c3ccc(N(c5ccccc5)c5ccccc5)cc3C4(C)c3ccccc3)cccc21 ; CC1(C)c2cc(N(c3ccccc3)c3ccccc3)ccc2-c2c(-c3cccc4c3-c3ccc(N(c5ccccc5)c5ccccc5)cc3C4(c3ccccc3)c3ccccc3)cccc21]

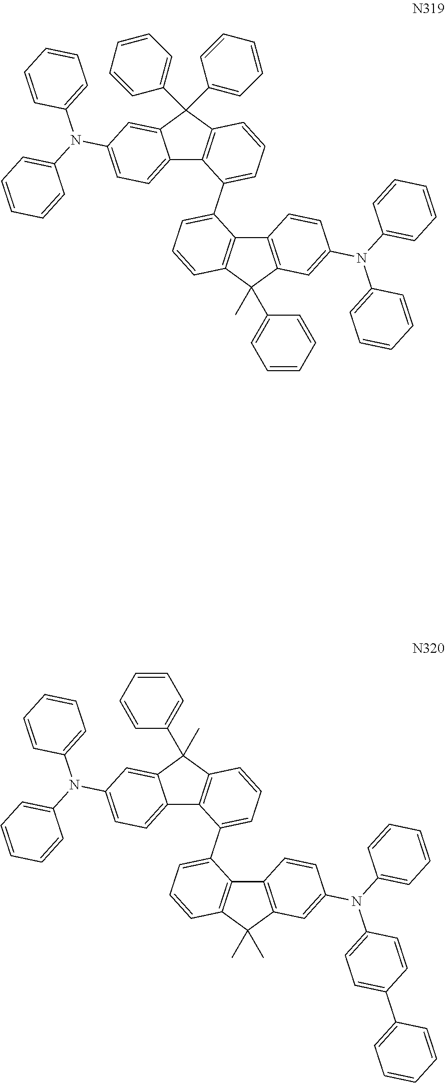 [SMILES: CC1(c2ccccc2)c2cc(N(c3ccccc3)c3ccccc3)ccc2-c2c(-c3cccc4c3-c3ccc(N(c5ccccc5)c5ccccc5)cc3C4(c3ccccc3)c3ccccc3)cccc21 ; CC1(C)c2cc(N(c3ccccc3)c3ccc(-c4ccccc4)cc3)ccc2-c2c(-c3cccc4c3-c3ccc(N(c5ccccc5)c5ccccc5)cc3C4(C)c3ccccc3)cccc21]

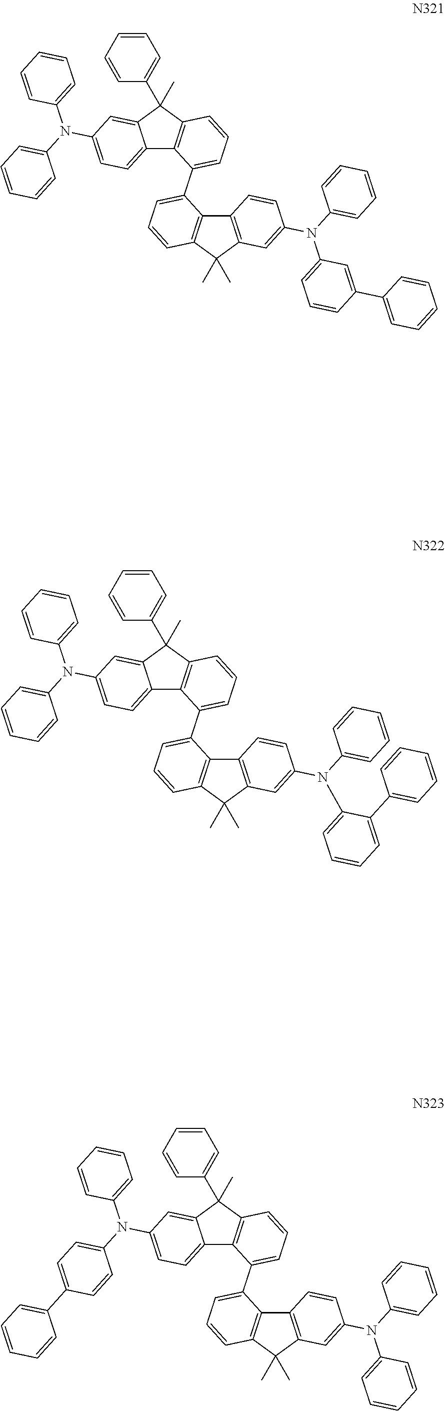 [SMILES: CC1(C)c2cc(N(c3ccccc3)c3cccc(-c4ccccc4)c3)ccc2-c2c(-c3cccc4c3-c3ccc(N(c5ccccc5)c5ccccc5)cc3C4(C)c3ccccc3)cccc21 ; CC1(C)c2cc(N(c3ccccc3)c3ccccc3-c3ccccc3)ccc2-c2c(-c3cccc4c3-c3ccc(N(c5ccccc5)c5ccccc5)cc3C4(C)c3ccccc3)cccc21 ; CC1(C)c2cc(N(c3ccccc3)c3ccccc3)ccc2-c2c(-c3cccc4c3-c3ccc(N(c5ccccc5)c5ccc(-c6ccccc6)cc5)cc3C4(C)c3ccccc3)cccc21]

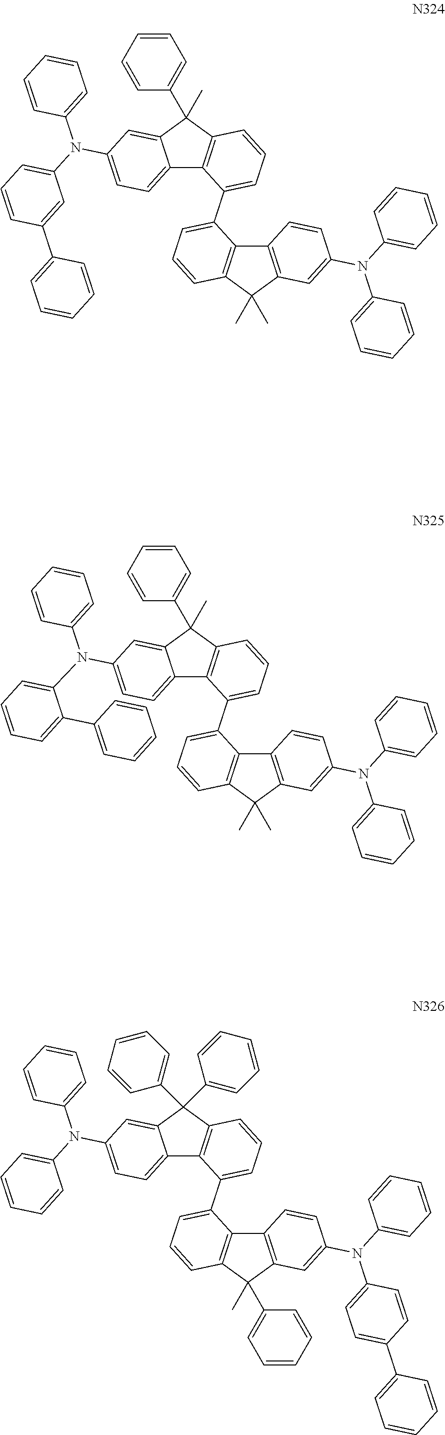 [SMILES: CC1(C)c2cc(N(c3ccccc3)c3ccccc3)ccc2-c2c(-c3cccc4c3-c3ccc(N(c5ccccc5)c5cccc(-c6ccccc6)c5)cc3C4(C)c3ccccc3)cccc21 ; CC1(C)c2cc(N(c3ccccc3)c3ccccc3)ccc2-c2c(-c3cccc4c3-c3ccc(N(c5ccccc5)c5ccccc5-c5ccccc5)cc3C4(C)c3ccccc3)cccc21 ; CC1(c2ccccc2)c2cc(N(c3ccccc3)c3ccc(-c4ccccc4)cc3)ccc2-c2c(-c3cccc4c3-c3ccc(N(c5ccccc5)c5ccccc5)cc3C4(c3ccccc3)c3ccccc3)cccc21]

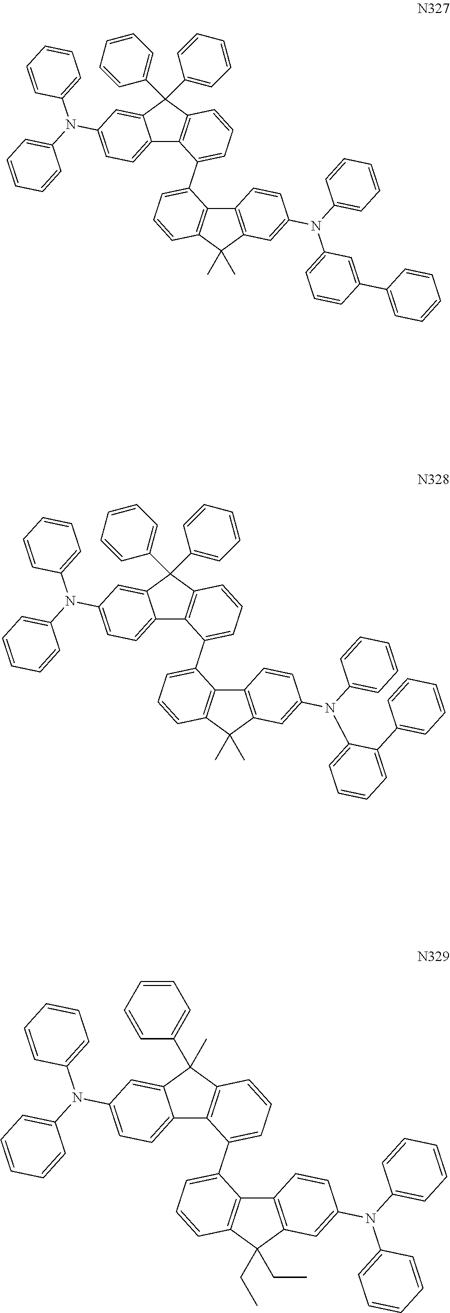 [SMILES: CC1(C)c2cc(N(c3ccccc3)c3cccc(-c4ccccc4)c3)ccc2-c2c(-c3cccc4c3-c3ccc(N(c5ccccc5)c5ccccc5)cc3C4(c3ccccc3)c3ccccc3)cccc21 ; CC1(C)c2cc(N(c3ccccc3)c3ccccc3-c3ccccc3)ccc2-c2c(-c3cccc4c3-c3ccc(N(c5ccccc5)c5ccccc5)cc3C4(c3ccccc3)c3ccccc3)cccc21 ; CCC1(CC)c2cc(N(c3ccccc3)c3ccccc3)ccc2-c2c(-c3cccc4c3-c3ccc(N(c5ccccc5)c5ccccc5)cc3C4(C)c3ccccc3)cccc21]

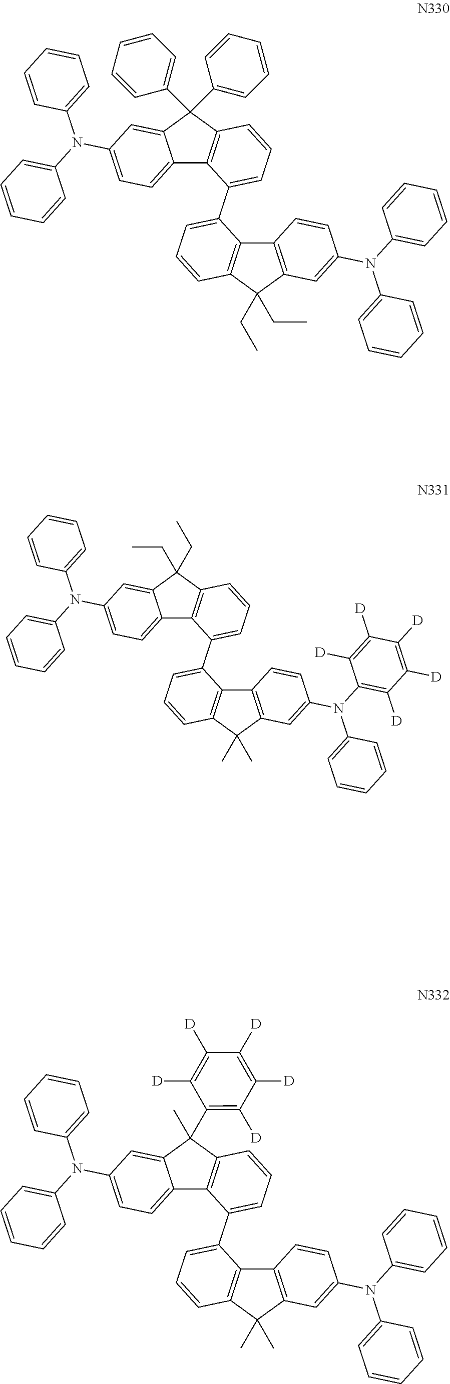 [SMILES: CCC1(CC)c2cc(N(c3ccccc3)c3ccccc3)ccc2-c2c(-c3cccc4c3-c3ccc(N(c5ccccc5)c5ccccc5)cc3C4(c3ccccc3)c3ccccc3)cccc21 ; [2H]c1c([2H])c([2H])c(N(c2ccccc2)c2ccc3c(c2)C(C)(C)c2cccc(-c4cccc5c4-c4ccc(N(c6ccccc6)c6ccccc6)cc4C5(CC)CC)c2-3)c([2H])c1[2H] ; [2H]c1c([2H])c([2H])c(C2(C)c3cc(N(c4ccccc4)c4ccccc4)ccc3-c3c(-c4cccc5c4-c4ccc(N(c6ccccc6)c6ccccc6)cc4C5(C)C)cccc32)c([2H])c1[2H]]

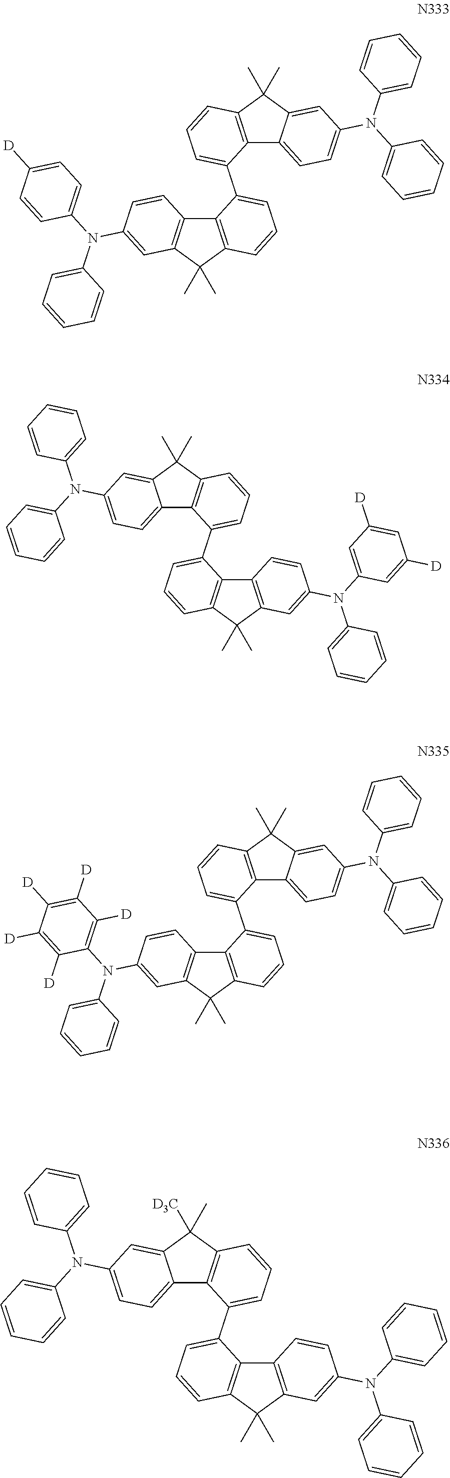 [SMILES: [2H]c1ccc(N(c2ccccc2)c2ccc3c(c2)C(C)(C)c2cccc(-c4cccc5c4-c4ccc(N(c6ccccc6)c6ccccc6)cc4C5(C)C)c2-3)cc1 ; [2H]c1cc([2H])cc(N(c2ccccc2)c2ccc3c(c2)C(C)(C)c2cccc(-c4cccc5c4-c4ccc(N(c6ccccc6)c6ccccc6)cc4C5(C)C)c2-3)c1 ; [2H]c1c([2H])c([2H])c(N(c2ccccc2)c2ccc3c(c2)C(C)(C)c2cccc(-c4cccc5c4-c4ccc(N(c6ccccc6)c6ccccc6)cc4C5(C)C)c2-3)c([2H])c1[2H] ; [2H]C([2H])([2H])C1(C)c2cc(N(c3ccccc3)c3ccccc3)ccc2-c2c(-c3cccc4c3-c3ccc(N(c5ccccc5)c5ccccc5)cc3C4(C)C)cccc21]

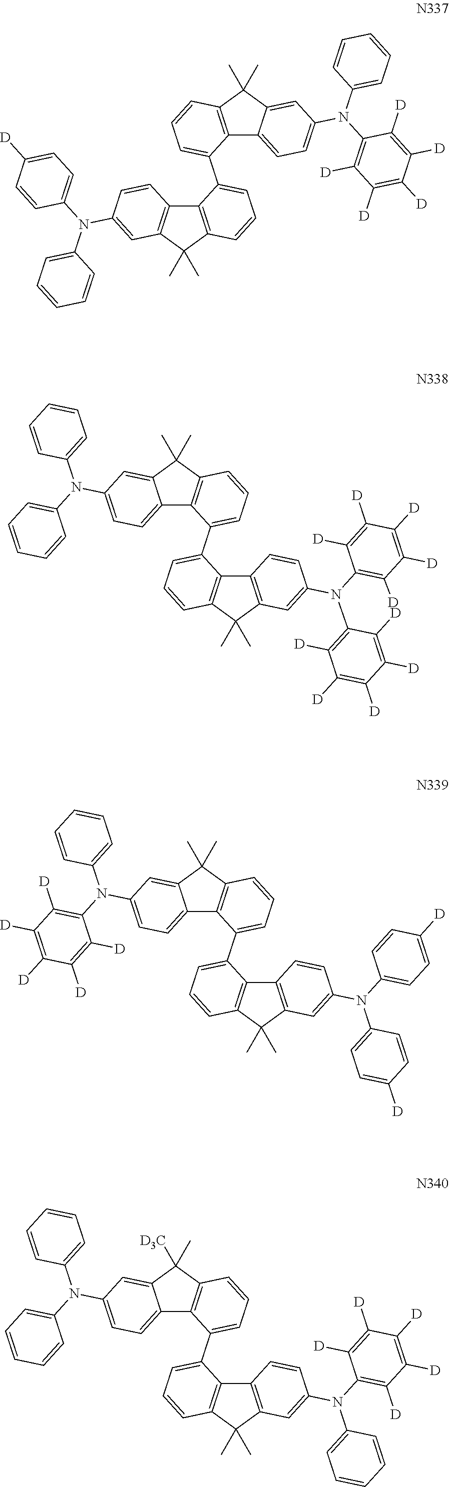

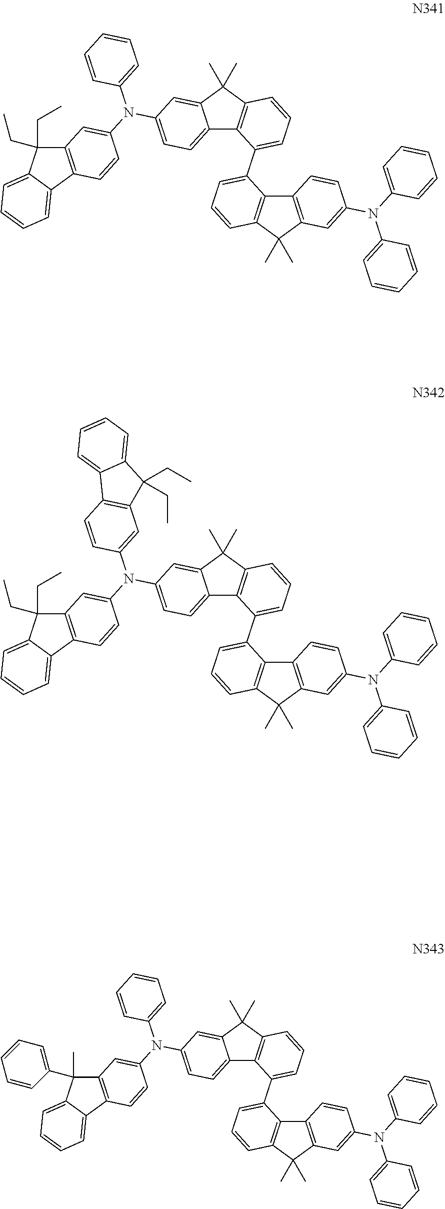 [SMILES: CCC1(CC)c2ccccc2-c2ccc(N(c3ccccc3)c3ccc4c(c3)C(C)(C)c3cccc(-c5cccc6c5-c5ccc(N(c7ccccc7)c7ccccc7)cc5C6(C)C)c3-4)cc21 ; CCC1(CC)c2ccccc2-c2ccc(N(c3ccc4c(c3)C(C)(C)c3cccc(-c5cccc6c5-c5ccc(N(c7ccccc7)c7ccccc7)cc5C6(C)C)c3-4)c3ccc4c(c3)C(CC)(CC)c3ccccc3-4)cc21 ; CC1(C)c2cc(N(c3ccccc3)c3ccccc3)ccc2-c2c(-c3cccc4c3-c3ccc(N(c5ccccc5)c5ccc6c(c5)C(C)(c5ccccc5)c5ccccc5-6)cc3C4(C)C)cccc21]

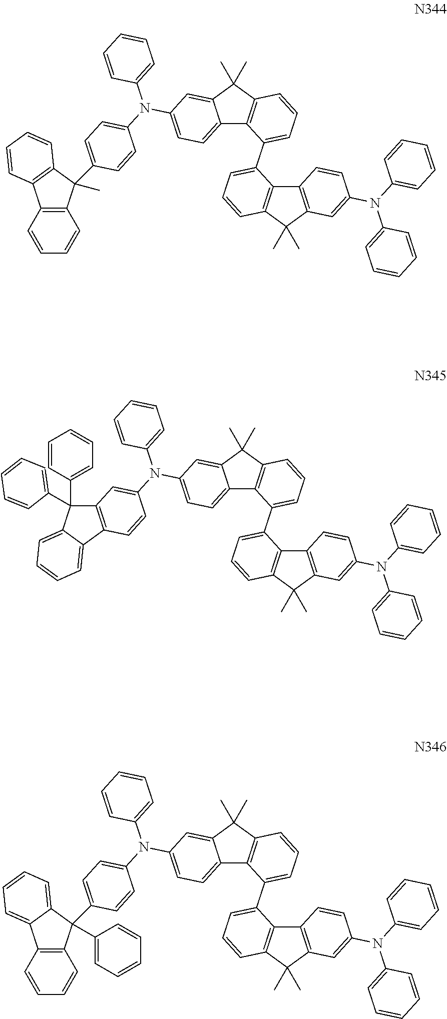 [SMILES: CC1(C)c2cc(N(c3ccccc3)c3ccccc3)ccc2-c2c(-c3cccc4c3-c3ccc(N(c5ccccc5)c5ccc(C6(C)c7ccccc7-c7ccccc76)cc5)cc3C4(C)C)cccc21 ; CC1(C)c2cc(N(c3ccccc3)c3ccccc3)ccc2-c2c(-c3cccc4c3-c3ccc(N(c5ccccc5)c5ccc6c(c5)C(c5ccccc5)(c5ccccc5)c5ccccc5-6)cc3C4(C)C)cccc21 ; CC1(C)c2cc(N(c3ccccc3)c3ccccc3)ccc2-c2c(-c3cccc4c3-c3ccc(N(c5ccccc5)c5ccc(C6(c7ccccc7)c7ccccc7-c7ccccc76)cc5)cc3C4(C)C)cccc21]

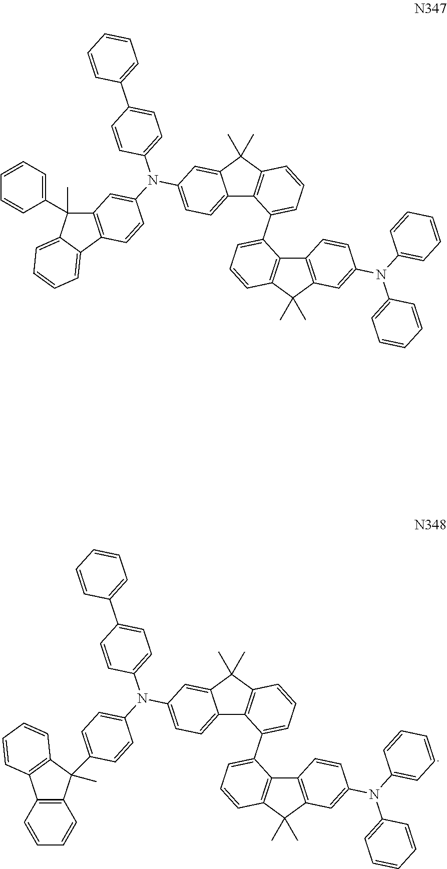 [SMILES: CC1(C)c2cc(N(c3ccccc3)c3ccccc3)ccc2-c2c(-c3cccc4c3-c3ccc(N(c5ccc(-c6ccccc6)cc5)c5ccc6c(c5)C(C)(c5ccccc5)c5ccccc5-6)cc3C4(C)C)cccc21 ; CC1(C)c2cc(N(c3ccccc3)c3ccccc3)ccc2-c2c(-c3cccc4c3-c3ccc(N(c5ccc(-c6ccccc6)cc5)c5ccc(C6(C)c7ccccc7-c7ccccc76)cc5)cc3C4(C)C)cccc21]

In the structure o the organic compound provided in the embodiments o the present disclosure, two fluorenyl groups are connected by a single bond to form an approximately orthogonal highly twisted conformation, and two fluorenyl groups are respectively connected to one aromatic amine group, so that the molecular structure is further twisted, the energy level distribution of the molecule is more uniform, and the hole transport capacity of the organic compound is stronger and the stability is higher, and the transport balance of the holes and the electrons in the light-emitting element are adjusted to be stronger, thereby improving the luminous efficiency of the light-emitting element and prolonging the service life of the light-emitting element.

The present disclosure also provides a mixture. The mixture includes at least one organic compound as described above and an organic functional material. The organic functional material is selected from at least one of a hole transport material, a hole injection material, a hole blocking material, an electron injection material, an electron transport material, a host material or a guest material.

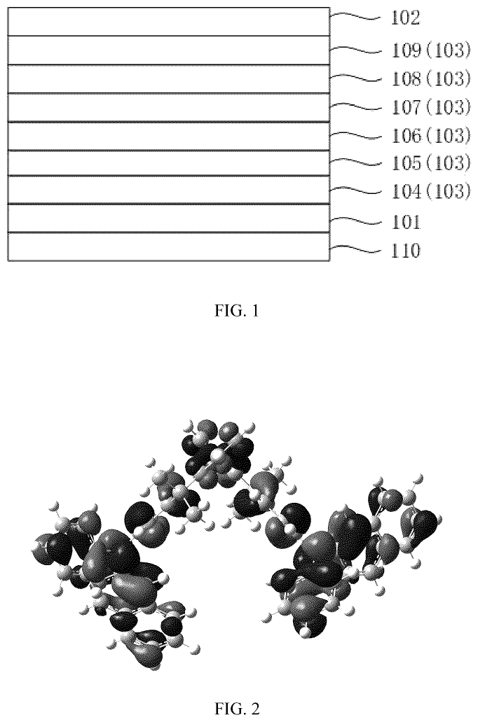

Referring to FIG. 1, the present disclosure further provides a light-emitting element including a pair of electrodes including a first electrode 101 and a second electrode 102; an organic functional layer 103 disposed between the first electrode 101 and the second electrode 102; wherein the material of the organic functional layer 103 includes one or more organic compounds as described above. The first electrode 101 may be an anode and the second electrode 102 may be a cathode.

In some embodiments, the light-emitting element may be used in an organic light-emitting diode, an organic photovoltaic cell, an organic light-emitting cell, an organic field-effect transistor, an organic light-emitting field-effect transistor, an organic laser, an organic spin-electron device, an organic sensor, an organic plasma-emitting diode, and the like. Preferred are organic light-emitting diodes, organic photovoltaic cells, and organic light-emitting cells.

In some embodiments, the light-emitting element may be applied to a variety of electronic devices, such as a display panel, a lighting equipment, a light source, and the like.

In some embodiments, the organic functional layer 103 may be a single layer. In this case, the organic functional layer 103 is a mixture layer including a first compound and a second compound. The first compound is selected from one or more of the organic compounds described above. The second compound is selected from one or more of a hole injection material, a hole transport material, an electron injection material, an electron transport material, a hole blocking material, a light emitting guest material, a light emitting host material, and an organic dye. The various organic functional materials included in the organic functional layer 103 are described in detail in WO2010135519A1, US20090134784A1, and WO2011110277A1, the entire contents of which are hereby incorporated herein by reference.

The light-emitting guest material is selected from a singlet emitter (a fluorescent emitter), a triplet emitter (a phosphorescent emitter), and a TADF material.

The organic compound may be used as hole transport materials.

In some embodiments, the organic functional layer 103 may include multiple layers. In a case that the organic functional layer 103 is multi-layered, the organic functional layer 103 at least includes a light-emitting layer 107. Preferably, the organic functional layer 103 includes a hole injection layer 104, a hole transport layer 105, a light-emitting auxiliary layer 106, a light-emitting layer 107, an electron injection layer 109, an electron transport layer 108, or a hole blocking layer.

In some embodiments, the hole transport layer 105 is disposed between the light-emitting layer 107 and the first electrode 101, the light-emitting auxiliary layer 106 is disposed between the hole transport layer 105 and the light-emitting layer 107, the hole injection layer 104 is disposed between the hole transport layer 105 and the first electrode 101, the electron transport layer 108 is disposed between the light-emitting layer 107 and the second electrode 102, and the electron injection layer 109 is disposed between the electron transport layer 108 and the second electrode 102.

In some embodiments, the light-emitting element may be a blue light-emitting element, a green light-emitting element, or a red light-emitting element. The light-emitting layer 107 may include a host material and a guest material. The guest material is one or more of the organic compounds described above, and the host material includes a fused aromatic derivative or a heteroaromatic compound.

The light-emitting wavelength of the light-emitting element ranges from 300 nm to 1000 nm. Further, the light-emitting wavelength of the light-emitting element ranges from 350 nm to 900 nm. Furthermore, the light-emitting wavelength of the light-emitting element ranges from 400 nm to 800 nm. Furthermore, the light-emitting wavelength of the light-emitting element is within the wavelength range of red light, the wavelength range of green light, or the wavelength range of blue light.

In some embodiments, the host materials include fused aromatic ring derivatives, heterocycle-containing compounds, and the like, such as at least one of anthracene derivatives, pyrene derivatives, naphthalene derivatives, pentacene derivatives, phenanthrene compounds, fluoranthene compounds, carbazole derivatives, dibenzofuran derivatives, ladder-type furan compounds, pyrimidine derivatives, and the like. The host materials may be host materials applied to a red light-emitting element, host materials applied to a green light-emitting element, or host materials applied to a blue light-emitting element. Preferably, the host materials are host materials applied to a blue light-emitting element. In a case that the host materials are host materials applied to a blue light-emitting element, and the host materials are preferably anthracene organic compounds.

In some embodiments, the mass ratio of the host materials to the guest materials is 99:1 to 70:30, such as 90:10, 85:15, 80:20, 75:25, and the like, preferably, it is 99:1 to 90:10, such as 97:3, 96:4, 95:5, 93:7, 92:8, and the like. The guest materials are dispersed in the host materials, and the mass ratio of the host material to the guest material ranges from 99:1 to 70:30, which is beneficial to inhibit crystallization of the light-emitting layer 107 and the concentration quenching of the guest materials due to high concentration, thereby improving the luminous efficiency of the light-emitting element.

In some embodiments, the anode is an electrode for injecting holes, and the anode may inject holes into the organic functional layer 103, for example, the anode injects holes into the hole injection layer, the hole transport layer, or the light-emitting layer. The anode may include at least one of conductive metals, conductive metal oxides, or conductive polymers. Preferably, the absolute value of the difference between the work function of the anode and HOMO (Highest Occupied Molecular Orbital) level or the valence band level of the light-emitting materials in the light-emitting layer or the p-type semiconductor materials serving as the hole injection layer or hole transport layer or light-emitting auxiliary layer is less than 0.5 eV, preferably less than 0.3 eV, and more preferably less than 0.2 eV. The anode materials include, but are not limited to, at least one of Al, Cu, Au, Ag, Mg, Fe, Co, Ni, Mn, Pd, Pt, ITO (Indium Tin Oxide), aluminum-doped zinc oxide (AZO), and the like, or other suitable and known anode materials, which can be readily selected by those skilled in the art. The anode materials may be deposited using any suitable technique, such as a suitable physical vapor deposition method, including radio frequency magnetron sputtering, vacuum thermal evaporation, e-beam, and the like. In some embodiments, the anode is patterned. Patterned ITO conductive substrates are commercially available and can be used to prepare devices according to the present disclosure.

In some embodiments, the cathode is an electrode for injecting electrons, and the cathode may inject electrons into the organic functional layer, for example, the cathode injects electrons into the electron injection layer, the electron transport layer, or the light-emitting layer. The cathode may include at least one of conductive metals or conductive metal oxides. Preferably, absolute value of the difference between the work function of the cathode and LUMO (Lowest Unoccupied Molecular Orbital) level or the valence band level of the light-emitting materials in the light-emitting layer or the p-type semiconductor materials serving as the electron injection layer or electron transport layer or hole blocking layer is less than 0.5 eV, preferably less than 0.3 eV, and more preferably less than 0.2 eV. All materials that can be used as cathodes for organic electronic devices may be used as cathode materials for the devices of the present disclosure. Examples of the cathode materials include, but are not limited to, at least one of Al, Au, Ag, Ca, Ba, Mg, LiF/Al, MgAg alloy, BaF₂/Al, Cu, Fe, Co, Ni, Mn, Pd, Pt, ITO, and the like. The cathode materials may be deposited using any suitable technique, such as a suitable physical vapor deposition method, including radio frequency magnetron sputtering, vacuum thermal evaporation, e-beam, and the like

In some embodiments, the hole injection layer 104 is used to facilitate hole injection from the anode to the light-emitting layer 107. The hole injection layer 104 includes a hole injection material that is a material that can receive holes injected from a positive electrode at a low voltage. Preferably, the highest occupied molecular orbital (HOMO) of the hole injection materials is between the work function of the material at the anode and the HOMO of the functional materials (such as the hole transport material of the hole transport layer) of the film layer away from the anode. The hole injection materials include, but are not limited to, at least one of metalloporphyrin, oligomeric thiophene, arylamine-based organic materials, hexanitrile-hexaazabenzophenanthrene-based organic materials, quinacridone-based organic materials, a perylene-based organic materials, anthraquinones, polyaniline-based and a polythiophene-based conductive polymers, and the like.

In some embodiments, the hole transport layer 105 may be used to transport holes to the light-emitting layer 107. The hole transport layer 105 includes a hole transport material that receives holes transported from the anode or the hole injection layer and transfers holes to the light-emitting layer. The hole transport materials may be selected from organic compounds as described above.

In some embodiments, the electron transport layer 108 is used to transport electrons. The electron transport layer 108 includes an electron transport material that receives electrons injected from a negative electrode and transfers electrons to the light-emitting layer 107. The electron transport materials are materials with high electron mobility known in the art. The electron transport materials may include, but are not limited to, at least one of an Al complex of 8-hydroxyquinoline, complexes including Alq3, organic radical compounds, hydroxyflavone-metal complexes, lithium 8-hydroxyquinoline (LiQ), and benzimidazole-based compounds.

In some embodiments, the electron injection layer 109 is used for injecting electrons. The electron injection layer 109 includes electron injection materials. The electron injection materials are preferably materials that have the ability to transport electrons, which have the effect of injecting electrons from the negative electrode, have an excellent effect of injecting electrons into the light-emitting layer 107 or the light-emitting materials, prevent excitons generated by the light-emitting layer 107 from moving to the hole injection layer, and also having an excellent ability to form a thin film. The electron injection materials include, but are not limited to, at least one of 8-hydroxyquinoline lithium (LiQ), fluorenone, anthraquinone dimethane, phenylbenzoquinone, thiopyran dioxide, oxazole, oxadiazole, triazole, imidazole, perylene tetracarboxylic acid, fluorenylidene methane, anthracenone, and the like and derivatives thereof, metal complex compounds, a nitrogen-containing 5-membered ring derivative, and the like.

In some embodiments, the hole blocking layer is used to block holes from reaching the negative electrode, and can generally formed under the same conditions as the hole injection layer 104. The hole blocking layer includes hole blocking materials, which include, but are not limited to, at least one of oxadiazole derivatives or triazole derivatives, phenanthroline derivatives, BCP, aluminum complexes, and the like.

In some embodiments, the light-emitting element further includes a substrate 110 on which the first electrode 101, the hole injection layer 104, the hole transport layer 105, the light-emitting auxiliary layer 106, the light-emitting layer 107, the electron transport layer 108, the electron injection layer 109, and the second electrode 102 are sequentially stacked. The substrate 110 may be a transparent substrate or an opaque substrate. In a case that the substrate 110 is a transparent substrate, a transparent light-emitting element may be fabricated. The substrate 110 may be a rigid substrate or a flexible substrate having elasticity. The material of the substrate 110 may include, but is not limited to, plastic, polymer, metal, semiconductor wafer, glass, and the like. Preferably, the substrate 110 includes at least one smooth surface for forming the anode on the surface. More preferably, the surface is free of surface defects. Preferably, the material of the substrate 110 is a polymeric film or plastic, including, but not limited to, polyethylene terephthalate (PET material) and polyethylene glycol (2,6-naphthalene) (PEN material). The glass transition temperature of the substrate 110 is greater than or equal to 150° C., preferably greater than or equal to 200° C., more preferably greater than or equal to 250° C., and most preferably greater than or equal to 300° C.

In some embodiments, the light-emitting element may be a solution-type light-emitting element, i.e., at least one of the organic functional layers is prepared by printing (e.g., inkjet printing).

In some embodiments, the material of the organic functional layer, the material of the mixture layer, or the material of the light-emitting layer may be prepared by a composition, and the preparation process may be formed by a printing or coating process. Printing or coating techniques include ink-jet printing, nozzle printing, letterpress printing, screen printing, dip coating, spin coating, doctor blade coating, roller printing, twist roller printing, flexographic lithographic printing, flexographic printing, rotary printing, spraying, brushing or pad printing, slot-type extrusion coating, and the like, wherein gravure printing, jet printing and inkjet are preferable.

The composition may be a solution or suspension, and the composition may include a dispersion and a dispersing agent. The dispersion is one or more of the organic compounds as described above, and the dispersant is used to disperse the dispersion.

In the composition, the mass fraction of the organic compound as described above may be from 0.3% to 30%, preferably from 0.5% to 20%, more preferably from 0.5% to 15%, further preferably from 0.5% to 10%, most preferably from 1% to 5%.

In a case that the composition is used in a printing process, the composition may be an ink, the viscosity and surface tension of the ink are important parameters, and suitable surface tension parameters of the ink are suitable for a specific substrate and a specific printing method. In some embodiments, the surface tension of the ink at the operating temperature or at 25° C. ranges from 19 dyne/cm to 50 dyne/cm; preferably from 22 dyne/cm to 35 dyne/cm; more preferably, from 25 dyne/cm to 33 dyne/cm, which is beneficial to application in the inkjet printing process. In some embodiments, the viscosity of the ink at the operating temperature or 25° C. ranges from 1 cps to 100 cps; preferably from 1 cps to 50 cps; more preferably from 1.5 cps to 20 cps; and most preferably from 4.0 cps to 20 cps, which is beneficial to application in the inkjet printing process.

In some embodiments, the dispersant has a hansen solubility parameter in the following ranges: δd (dispersion force) ranges from 17.0 MPa^{1/2} to 23.2 MPa^{1/2}, preferably from 18.5 MPa^{1/2} to 21.0 MPa^{1/2}; Δp (polarity force) ranges from 0.2 MPa^{1/2} to 12.5 MPa^{1/2}, preferably from 2.0 MPa^{1/2} to 6.0 MPa^{1/2}; Δh (hydrogen bonding force) ranges from 0.9 MPa^{1/2} to 14.2 MPa^{1/2}, preferably from 2.0 MPa^{1/2} to 6.0 MPa^{1/2}.

In some embodiments, the boiling point of the organic solvent is greater than or equal to 150° C.; preferably greater than or equal to 180° C.; more preferably greater than or equal to 200° C.; yet more preferably 250° C.; further preferably greater than or equal to 275° C., and most preferably greater than or equal to 300° C. The boiling point of the organic solvent is at least greater than or equal to 150° C., which is beneficial to prevent nozzle clogging of inkjet print heads, and the higher the boiling point is, the better it is to prevent clogging.

The dispersant may include at least one organic solvent that may be evaporated from the solvent system to form a film including the functional materials. The organic solvent may include at least one first organic solvent, and the first organic solvent may be selected from aromatic or heteroaromatic-based solvents. Specifically, the first organic solvent may be selected from p-diisopropylbenzene, pentylbenzene, tetrahydronaphthalene, cyclohexylbenzene, chloronaphthalene, 1, 4-dimethylnaphthalene, 3-isopropylbiphenyl, p-methylcumene, dipentylbenzene, tripentylbenzene, amyl-toluene, o-diethylbenzene, m-diethylbenzene, p-diethylbenzene, 1,2,3,4-tetramethylbenzene, 1,2,3,5-tetramethylbenzene, 1,2,4,5-tetramethylbenzene, butylbenzene, dodecylbenzene, dihexylbenzene, dibutylbenzene, p-diisopropylbenzene, cyclohexylbenzene, benzylbutylbenzene, dimethylnaphthalene, 3-isopropylbiphenyl, p-methylcumene, 1-methylnaphthalene, 1,2,4-trichlorobenzene, 4,4-difluorodiphenylmethane, 1,2-dimethoxy-4-(1-propenyl)benzene, diphenylmethane, 2-phenylpyridine, 3-phenylpyridine, N-methyldiphenylamine, 4-isopropylbiphenyl, α,α-dichlorodiphenylmethane, 4-(3-phenylpropyl)pyridine, benzyl benzoate, 1,1-bis(3,4-dimethylphenyl)ethane, 2-isopropylnaphthalene, quinoline, isoquinoline, methyl 2-furanoate, ethyl 2-furanoate, and the like.

The first organic solvent may be selected from aromatic ketone-based solvents. Specifically, the first organic solvent may be selected from 1-tetralone, 2-tetralone, 2-(phenylepoxy)tetralone, 6-(methoxy) tetralone, acetophenone, propiophenone, benzophenone, and derivatives thereof, such as 4-methylacetophenone, 3-methylacetophenone, 2-methylacetophenone, 4-methylacetophenone, 3-methylacetophenone, 2-methylacetophenone, and the like.

The first organic solvent may be selected from aromatic ether-based solvents. Specifically, the first organic solvent may be selected from 3-phenoxytoluene, butoxybenzene, p-anisaldehyde dimethyl acetal, tetrahydro-2-phenoxy-2H-pyran, 1,2-dimethoxy-4-(1-propenyl)benzene, 1,4-benzodioxane, 1,3-dipropylbenzene, 2,5-dimethoxytoluene, 4-ethylbenzoethyl ether, 1,3-dipropoxybenzene, 1,2,4-trimethoxybenzene, 4-(1-propenyl)-1,2-dimethoxybenzene, 1,3-dimethoxybenzene, glycidyl phenyl ether, dibenzyl ether, 4-tert-butyl anisole, trans-p-propenyl anisole, 1,2-dimethoxybenzene, 1-methoxynaphthalene, diphenyl ether, 2-phenoxymethyl ether, 2-phenoxytetrahydrofuran, ethyl-2-naphthyl ether, and the like.

The first organic solvent may be selected from aliphatic ketone-based solvents. Specifically, the first organic solvent may be selected from aliphatic ketones, for example, 2-nonanone, 3-nonanone, 5-nonanone, 2-decanone, 2,5-hexanedione, 2,6,8-trimethyl-4-nonanone, fenchone, phorone, isophorone, di-n-amyl ketone, and the like; or aliphatic ethers, such as amyl ether, hexyl ether, dioctyl ether, ethylene glycol dibutyl ether, diethylene glycol diethyl ether, diethylene glycol butyl methyl ether, diethylene glycol dibutyl ether, triethylene glycol dimethyl ether, triethylene glycol ethyl methyl ether, triethylene glycol butyl methyl ether, tripropylene glycol dimethyl ether, tetraethylene glycol dimethyl ether, and the like.

The first organic solvent may be selected from organic ester-based solvents. Specifically, the first solvent may be selected from alkyl octanoate, alkyl sebacate, alkyl stearate, alkyl benzoate, alkyl phenylacetate, alkyl cinnamate, alkyl oxalate, alkyl maleate, alkyl lactone, alkyl oleate, and the like. Octyl octanoate, diethyl sebacate, diallyl phthalate, isononyl isononanoate, and the like are particularly preferred.

The organic solvent may also include a second organic solvent. The second organic solvent may be selected from one or more of methanol, ethanol, 2-methoxyethanol, dichloromethane, trichloromethane, chlorobenzene, o-dichlorobenzene, tetrahydrofuran, anisole, morpholine, toluene, o-xylene, m-xylene, p-xylene, 1, 4-dioxane, acetone, methyl ethyl ketone, 1,2-dichloroethane, 3-phenoxytoluene, 1,1,1-trichloroethane, 1,1,2,2-tetrachloroethane, ethyl acetate, butyl acetate, dimethylformamide, dimethylacetamide, dimethylsulfoxide, tetrahydronaphthalene, decalin, and indene.

In addition to the dispersoid and dispersant, the composition may also include may additionally include one or more components such as surface active compounds, lubricants, wetting agents, dispersing agents, hydrophobic agents, adhesives, and the like, for adjusting viscosity, film-forming performance, for adjusting viscosity, film-forming performance, improving adhesion, and the like.

Exemplary preparation methods of the organic compounds provided by the present disclosure are shown in the following exemplary examples 1 to 39.

### Synthesis of intermediates I-1 to I-8

Synthetic route of intermediate I-1 is as follows:

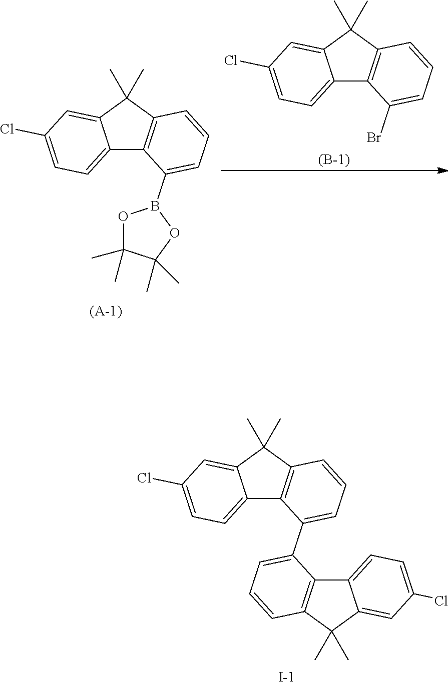

### Synthesis of intermediate I-1

A-1 (71.0 g, 0.2 mol) and B-1 (61.5 g, 0.2 mol) were weighed and added into a clean three-necked flask, and Pd(PPh₃)₄ (2.3 g, 2.0 mmol), potassium carbonate (55.3 g, 0.4 mol), toluene (400 mL), ethanol (100 mL), and deionized water (100 mL) were added therein. Air in the three-necked flask was displaced with nitrogen and the temperature is heated to 80° C. to reflux and react for 12 hours to obtain a mixture. After naturally cooling, the mixture was washed with water for separation, wherein the organic phase was dried and then spun to dryness, and purified through column chromatography to obtain the intermediate I-1 with a yield of 85.9%. Atmospheric solids analysis probe mass spectrum (ASAP-MS) result of the intermediate I-1 is as follows: MS (ASAP)=455[M+H]⁺.

### Synthesis of Intermediates I-2 to I-8

Each intermediate I-X was synthesized in the same way as intermediate I-1, except that substrate A-1 was replaced by each A-a, and substrate B-1 was replaced by each B-b, wherein a, b, and X respectively represent positive integers. The molecular structure, mass spectrum results, and yield of each intermediate I-X synthesized were shown in Table 1.

TABLE 1
Molecular structure, mass spectrometry results and yield of Intermediate I-2 to I-8

| | | | MS | |
|---|---|---|---|---|
| | | | (ASAP) | |
| | | | [M + | Yield |
| A-a | B-b | Intermediate I-X | H]⁺ | (%) |
| 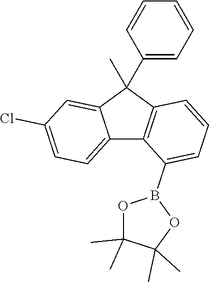 [SMILES: CC1(c2ccccc2)c2cc(Cl)ccc2-c2c(B3OC(C)(C)C(C)(C)O3)cccc21] (A-2) | 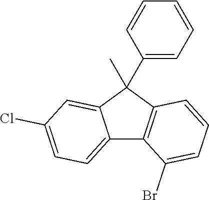 [SMILES: CC1(c2ccccc2)c2cc(Cl)ccc2-c2c(Br)cccc21] (B-2) | 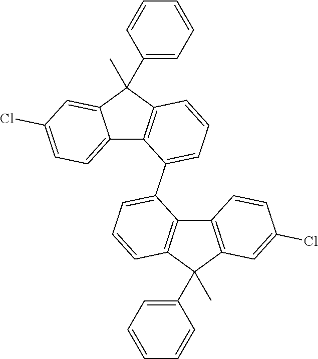 [SMILES: CC1(c2ccccc2)c2cc(Cl)ccc2-c2c(-c3cccc4c3-c3ccc(Cl)cc3C4(C)c3ccccc3)cccc21] I-2 | 580 | 79.5% |
| 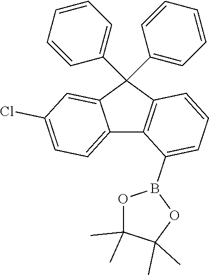 [SMILES: CC1(C)OB(c2cccc3c2-c2ccc(Cl)cc2C3(c2ccccc2)c2ccccc2)OC1(C)C] (A-3) | 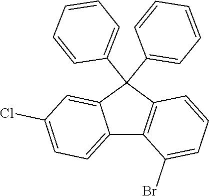 [SMILES: Clc1ccc2c(c1)C(c1ccccc1)(c1ccccc1)c1cccc(Br)c1-2] (B-3) | 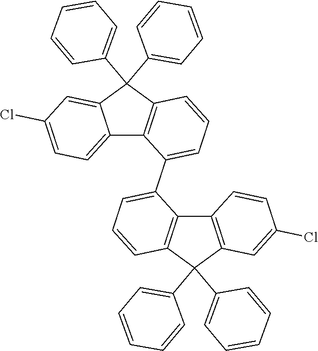 [SMILES: Clc1ccc2c(c1)C(c1ccccc1)(c1ccccc1)c1cccc(-c3cccc4c3-c3ccc(Cl)cc3C4(c3ccccc3)c3ccccc3)c1-2] I-3 | 704 | 76.7% |
| 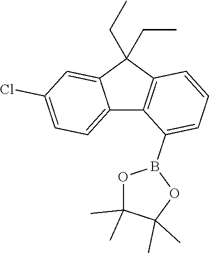 [SMILES: CCC1(CC)c2cc(Cl)ccc2-c2c(B3OC(C)(C)C(C)(C)O3)cccc21] (A-4) | 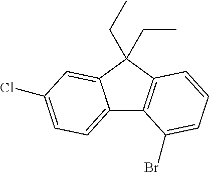 [SMILES: CCC1(CC)c2cc(Cl)ccc2-c2c(Br)cccc21] (B-4) | 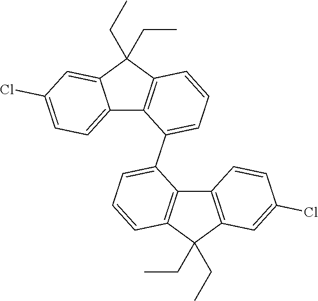 [SMILES: CCC1(CC)c2cc(Cl)ccc2-c2c(-c3cccc4c3-c3ccc(Cl)cc3C4(CC)CC)cccc21] I-4 | 512 | 81.8% |
| 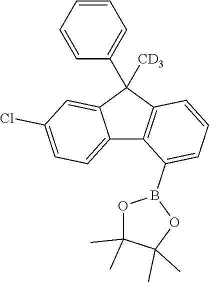 [SMILES: [2H]C([2H])([2H])C1(c2ccccc2)c2cc(Cl)ccc2-c2c(B3OC(C)(C)C(C)(C)O3)cccc21] (A-5) | 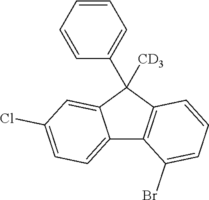 [SMILES: [2H]C([2H])([2H])C1(c2ccccc2)c2cc(Cl)ccc2-c2c(Br)cccc21] (B-5) | 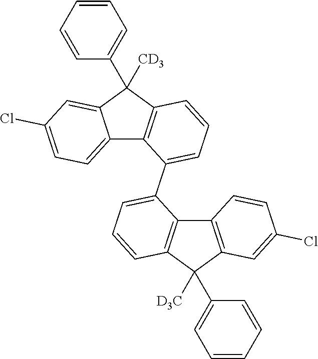 [SMILES: [2H]C([2H])([2H])C1(c2ccccc2)c2cc(Cl)ccc2-c2c(-c3cccc4c3-c3ccc(Cl)cc3C4(c3ccccc3)C([2H])([2H])[2H])cccc21] I-5 | 586 | 76.9% |
| 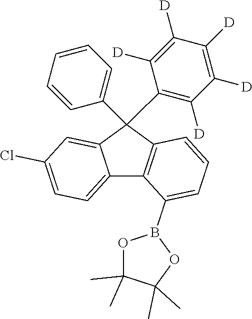 [SMILES: [2H]c1c([2H])c([2H])c(C2(c3ccccc3)c3cc(Cl)ccc3-c3c(B4OC(C)(C)C(C)(C)O4)cccc32)c([2H])c1[2H]] (A-6) | 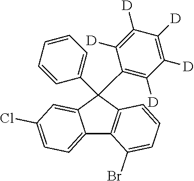 [SMILES: [2H]c1c([2H])c([2H])c(C2(c3ccccc3)c3cc(Cl)ccc3-c3c(Br)cccc32)c([2H])c1[2H]] (B-6) | 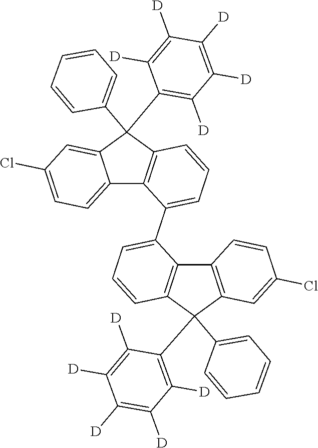 [SMILES: [2H]c1c([2H])c([2H])c(C2(c3ccccc3)c3cc(Cl)ccc3-c3c(-c4cccc5c4-c4ccc(Cl)cc4C5(c4ccccc4)c4c([2H])c([2H])c([2H])c([2H])c4[2H])cccc32)c([2H])c1[2H]] I-6 | 714 | 77.3% |
| 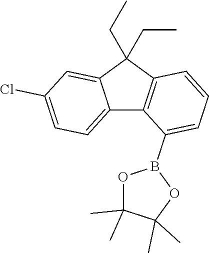 [SMILES: CCC1(CC)c2cc(Cl)ccc2-c2c(B3OC(C)(C)C(C)(C)O3)cccc21] (A-4) | 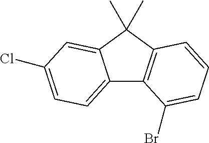 [SMILES: CC1(C)c2cc(Cl)ccc2-c2c(Br)cccc21] (B-1) | 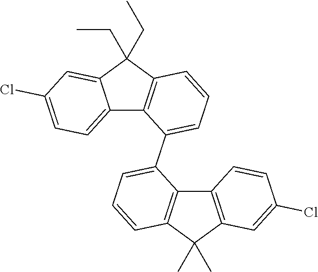 [SMILES: CCC1(CC)c2cc(Cl)ccc2-c2c(-c3cccc4c3-c3ccc(Cl)cc3C4(C)C)cccc21] I-7 | 484 | 72.8% |
| 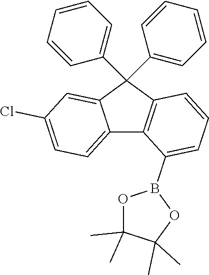 [SMILES: CC1(C)OB(c2cccc3c2-c2ccc(Cl)cc2C3(c2ccccc2)c2ccccc2)OC1(C)C] (A-3) | 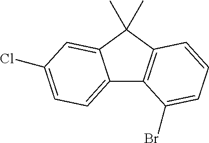 [SMILES: CC1(C)c2cc(Cl)ccc2-c2c(Br)cccc21] (B-1) | 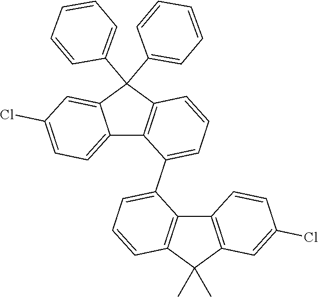 [SMILES: CC1(C)c2cc(Cl)ccc2-c2c(-c3cccc4c3-c3ccc(Cl)cc3C4(c3ccccc3)c3ccccc3)cccc21] 1-8 | 580 | 73.1% |

### Example 1

### Synthesis of Organic Compound M1

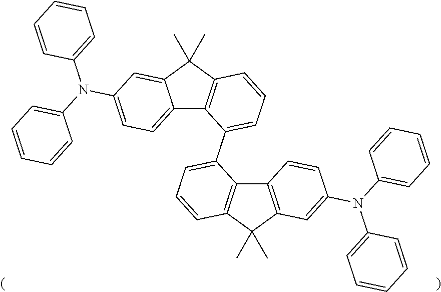 [SMILES: CC1(C)c2cc(N(c3ccccc3)c3ccccc3)ccc2-c2c(-c3cccc4c3-c3ccc(N(c5ccccc5)c5ccccc5)cc3C4(C)C)cccc21]

Synthetic route of organic compound M1 is as follows:

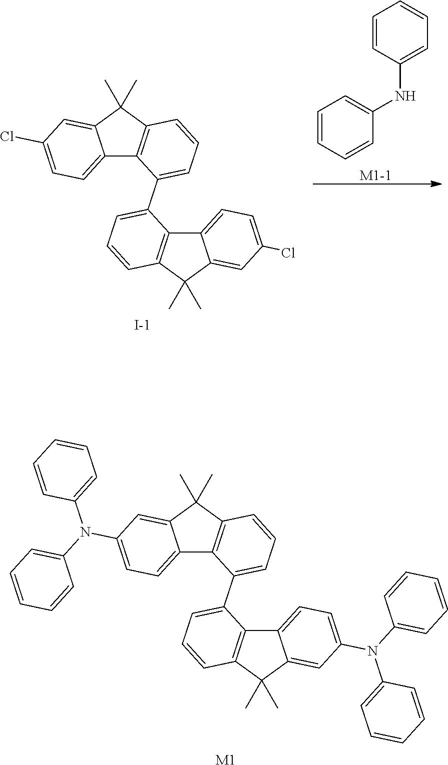

The specific synthetic steps of organic compound M1 are as follows:

I-1 (10.0 g, 0.02 mol) and M1-1 (8.5 g, 0.05 mol) were weighed and added into a clean three-necked flask, and Pd₂(dba)₃ (1%, 0.18 g), X-Phos (1.0 g, 2.0 mmol), sodium tert-butoxide (5.8 g, 0.06 mol), and anhydrous toluene (300 mL) were added therein. Air in the three-necked flask was displaced with nitrogen and the temperature is heated to 120° C. to reflux and react for 12 hours to obtain a mixture. After naturally cooling, the mixture was washed with water for separation, wherein the organic phase was dried and then spun to dryness, and purified through column chromatography to obtain the intermediate M1 with a yield of 79.2%. MS (ASAP)=721[M+H]+.

### Examples 2 to 26

In embodiments 2 to 26, each organic compound MY was synthesized in the same way as M1, except that I-1 was replaced by different intermediates I-X, and M1-1 was replaced by MY-1, wherein Y is a positive integer. The molecular structure, mass spectrum results, and yield of each organic compound MY were shown in Table 2.

TABLE 2
Molecular structure, mass spectrometry results and yield of organic compound MY

| Ex- | | | | MS | |
|---|---|---|---|---|---|
| am- | | | | (ASAP) | Yield |
| ples | I-X | MY-1 | Organic compound MY | [M + H]⁺ | (%) |
| 2 | I-1 | 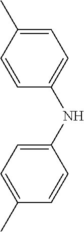 M5-1 | 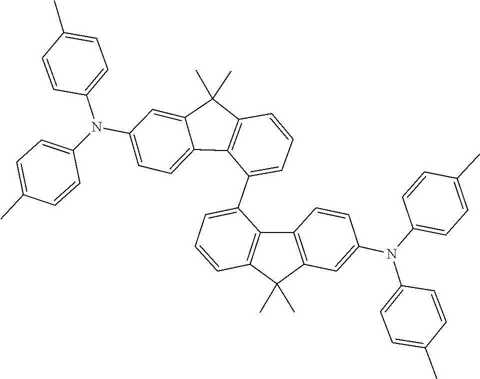 M5 | 777 | 76.1% |
| 3 | I-1 | 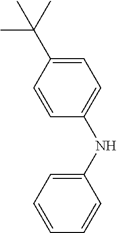 [SMILES: CC(C)(C)c1ccc(Nc2ccccc2)cc1] M24-1 | 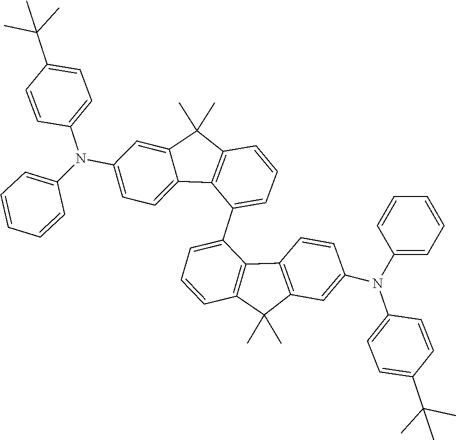 [SMILES: CC(C)(C)c1ccc(N(c2ccccc2)c2ccc3c(c2)C(C)(C)c2cccc(-c4cccc5c4-c4ccc(N(c6ccccc6)c6ccc(C(C)(C)C)cc6)cc4C5(C)C)c2-3)cc1] M24 | 833 | 74.8% |
| 4 | I-1 | 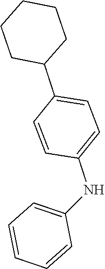 [SMILES: c1ccc(Nc2ccc(C3CCCCC3)cc2)cc1] M34-1 | 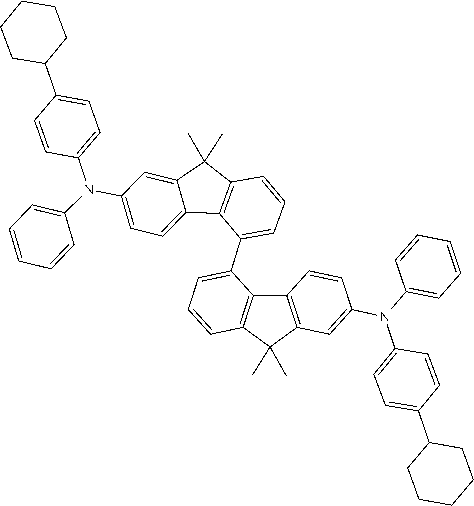 [SMILES: CC1(C)c2cc(N(c3ccccc3)c3ccc(C4CCCCC4)cc3)ccc2-c2c(-c3cccc4c3-c3ccc(N(c5ccccc5)c5ccc(C6CCCCC6)cc5)cc3C4(C)C)cccc21] M34 | 885 | 75.6% |
| 5 | I-1 | 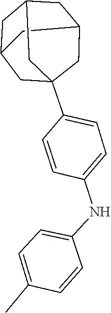 [SMILES: Cc1ccc(Nc2ccc(C34CC5CC(CC(C5)C3)C4)cc2)cc1] M39-1 | 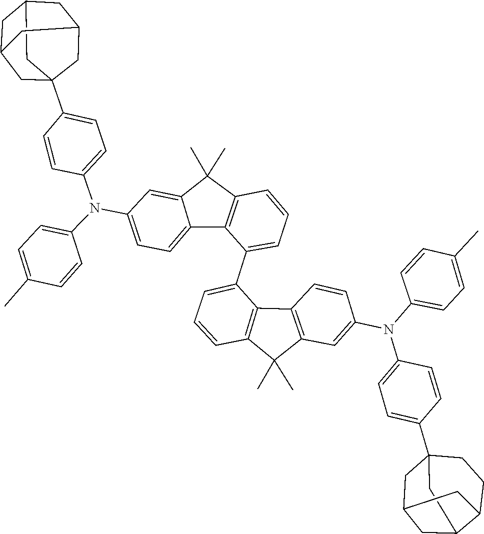 [SMILES: Cc1ccc(N(c2ccc(C34CC5CC(CC(C5)C3)C4)cc2)c2ccc3c(c2)C(C)(C)c2cccc(-c4cccc5c4-c4ccc(N(c6ccc(C)cc6)c6ccc(C78CCC9CC(CC9C7)C8)cc6)cc4C5(C)C)c2-3)cc1] M39 | 1018 | 71.0% |
| 6 | I-1 | 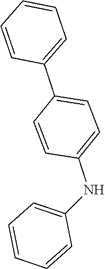 [SMILES: c1ccc(Nc2ccc(-c3ccccc3)cc2)cc1] M42-1 | 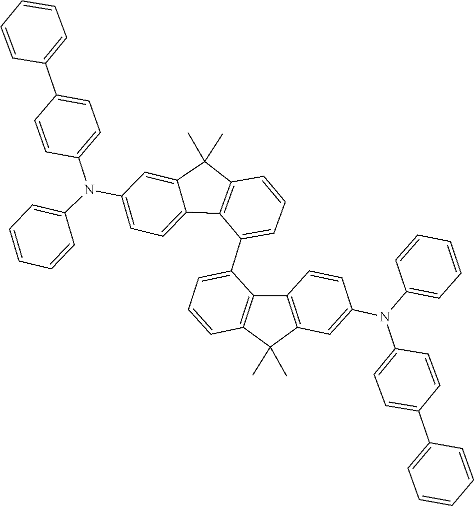 [SMILES: CC1(C)c2cc(N(c3ccccc3)c3ccc(-c4ccccc4)cc3)ccc2-c2c(-c3cccc4c3-c3ccc(N(c5ccccc5)c5ccc(-c6ccccc6)cc5)cc3C4(C)C)cccc21] M42 | 873 | 78.8% |
| 7 | I-1 | 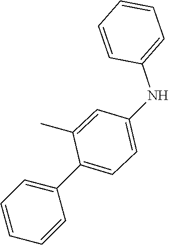 [SMILES: Cc1cc(Nc2ccccc2)ccc1-c1ccccc1] M73-1 | 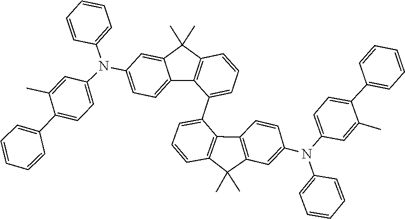 [SMILES: Cc1cc(N(c2ccccc2)c2ccc3c(c2)C(C)(C)c2cccc(-c4cccc5c4-c4ccc(N(c6ccccc6)c6ccc(-c7ccccc7)c(C)c6)cc4C5(C)C)c2-3)ccc1-c1ccccc1] M73 | 901 | 70.9% |
| 8 | I-1 | 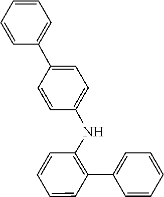 [SMILES: c1ccc(-c2ccc(Nc3ccccc3-c3ccccc3)cc2)cc1] M91-1 | 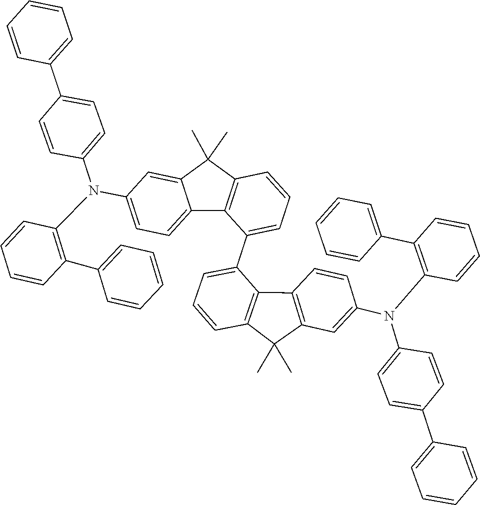 [SMILES: CC1(C)c2cc(N(c3ccc(-c4ccccc4)cc3)c3ccccc3-c3ccccc3)ccc2-c2c(-c3cccc4c3-c3ccc(N(c5ccc(-c6ccccc6)cc5)c5ccccc5-c5ccccc5)cc3C4(C)C)cccc21] M91 | 1025 | 70.1% |
| 9 | I-1 | 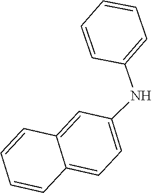 [SMILES: c1ccc(Nc2ccc3ccccc3c2)cc1] M95-1 | 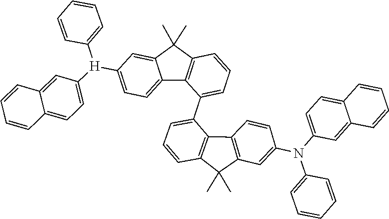 M95 | 821 | 76.8% |
| 10 | I-1 | 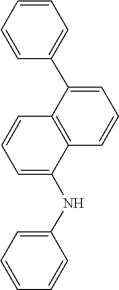 [SMILES: c1ccc(Nc2cccc3c(-c4ccccc4)cccc23)cc1] M121-1 | 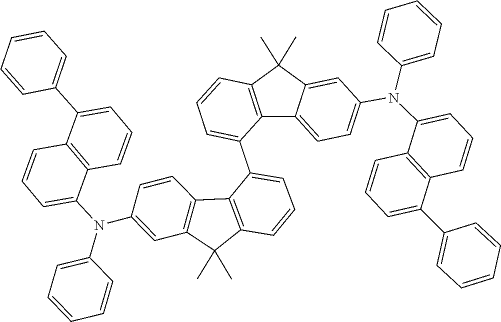 [SMILES: CC1(C)c2cc(N(c3ccccc3)c3cccc4c(-c5ccccc5)cccc34)ccc2-c2c(-c3cccc4c3-c3ccc(N(c5ccccc5)c5cccc6c(-c7ccccc7)cccc56)cc3C4(C)C)cccc21] M121 | 973 | 67.9% |
| 11 | I-1 | 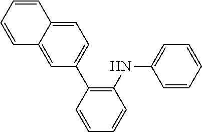 [SMILES: c1ccc(Nc2ccccc2-c2ccc3ccccc3c2)cc1] M124-1 | 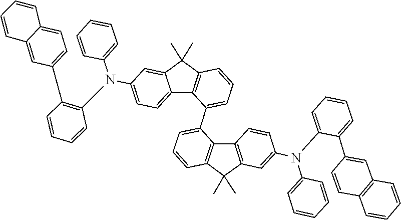 [SMILES: CC1(C)c2cc(N(c3ccccc3)c3ccccc3-c3ccc4ccccc4c3)ccc2-c2c(-c3cccc4c3-c3ccc(N(c5ccccc5)c5ccccc5-c5ccc6ccccc6c5)cc3C4(C)C)cccc21] M124 | 973 | 61.1% |
| 12 | I-1 | 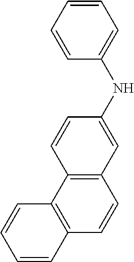 [SMILES: c1ccc(Nc2ccc3c(ccc4ccccc43)c2)cc1] M131-1 | 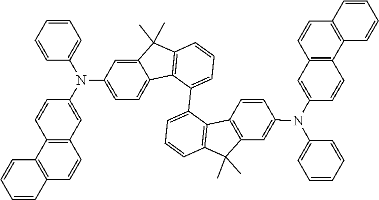 [SMILES: CC1(C)c2cc(N(c3ccccc3)c3ccc4c(ccc5ccccc54)c3)ccc2-c2c(-c3cccc4c3-c3ccc(N(c5ccccc5)c5ccc6c(ccc7ccccc76)c5)cc3C4(C)C)cccc21] M131 | 921 | 73.3% |
| 13 | I-1 | 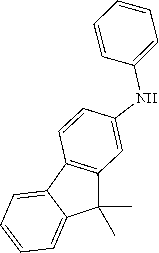 [SMILES: CC1(C)c2ccccc2-c2ccc(Nc3ccccc3)cc21] M138-1 | 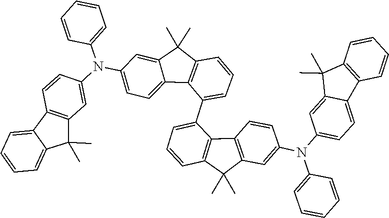 [SMILES: CC1(C)c2ccccc2-c2ccc(N(c3ccccc3)c3ccc4c(c3)C(C)(C)c3cccc(-c5cccc6c5-c5ccc(N(c7ccccc7)c7ccc8c(c7)C(C)(C)c7ccccc7-8)cc5C6(C)C)c3-4)cc21] M138 | 953 | 80.4% |
| 14 | I-1 | 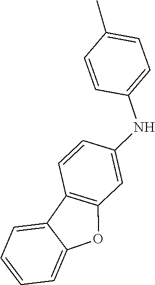 [SMILES: Cc1ccc(Nc2ccc3c(c2)oc2ccccc23)cc1] M158-1 | 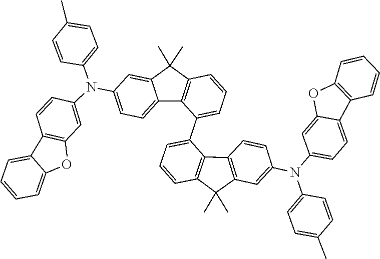 [SMILES: Cc1ccc(N(c2ccc3c(c2)C(C)(C)c2cccc(-c4cccc5c4-c4ccc(N(c6ccc(C)cc6)c6ccc7c(c6)oc6ccccc67)cc4C5(C)C)c2-3)c2ccc3c(c2)oc2ccccc23)cc1] M158 | 929 | 79.1% |
| 15 | I-1 | 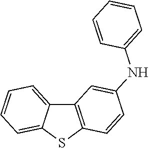 [SMILES: c1ccc(Nc2ccc3sc4ccccc4c3c2)cc1] M171-1 | 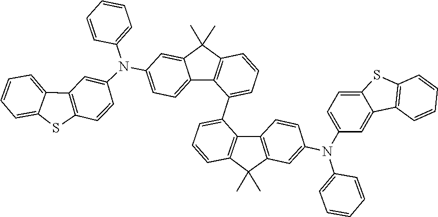 [SMILES: CC1(C)c2cc(N(c3ccccc3)c3ccc4sc5ccccc5c4c3)ccc2-c2c(-c3cccc4c3-c3ccc(N(c5ccccc5)c5ccc6sc7ccccc7c6c5)cc3C4(C)C)cccc21] M171 | 933 | 83.7% |
| 16 | I-1 | 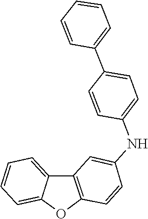 [SMILES: c1ccc(-c2ccc(Nc3ccc4oc5ccccc5c4c3)cc2)cc1] M187-1 | 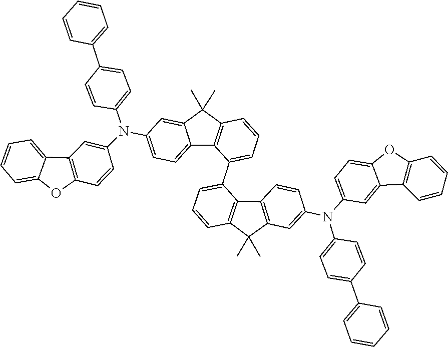 [SMILES: CC1(C)c2cc(N(c3ccc(-c4ccccc4)cc3)c3ccc4oc5ccccc5c4c3)ccc2-c2c(-c3cccc4c3-c3ccc(N(c5ccc(-c6ccccc6)cc5)c5ccc6oc7ccccc7c6c5)cc3C4(C)C)cccc21] M187 | 1053 | 73.8% |
| 17 | I-1 | 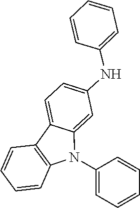 [SMILES: c1ccc(Nc2ccc3c4ccccc4n(-c4ccccc4)c3c2)cc1] M211-1 | 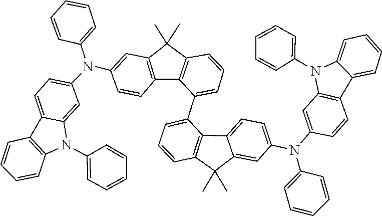 [SMILES: CC1(C)c2cc(N(c3ccccc3)c3ccc4c5ccccc5n(-c5ccccc5)c4c3)ccc2-c2c(-c3cccc4c3-c3ccc(N(c5ccccc5)c5ccc6c7ccccc7n(-c7ccccc7)c6c5)cc3C4(C)C)cccc21] M211 | 1051 | 78.1% |
| 18 | I-1 | 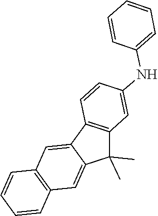 [SMILES: CC1(C)c2cc(Nc3ccccc3)ccc2-c2cc3ccccc3cc21] M222-1 | 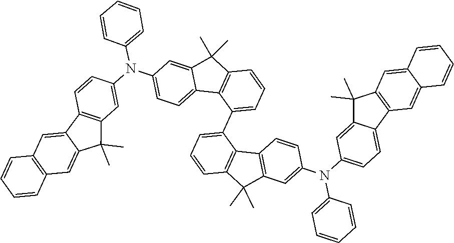 [SMILES: CC1(C)c2cc(N(c3ccccc3)c3ccc4c(c3)C(C)(C)c3cccc(-c5cccc6c5-c5ccc(N(c7ccccc7)c7ccc8c(c7)C(C)(C)c7cc9ccccc9cc7-8)cc5C6(C)C)c3-4)ccc2-c2cc3ccccc3cc21] M222 | 1053 | 77.8% |
| 19 | I-1 | 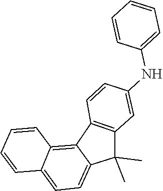 [SMILES: CC1(C)c2cc(Nc3ccccc3)ccc2-c2c1ccc1ccccc21] M232-1 | 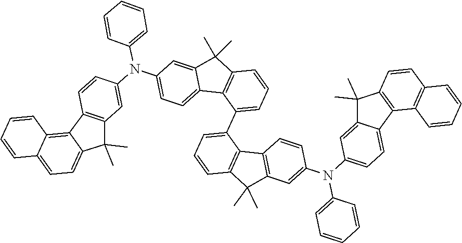 [SMILES: CC1(C)c2cc(N(c3ccccc3)c3ccc4c(c3)C(C)(C)c3ccc5ccccc5c3-4)ccc2-c2c(-c3cccc4c3-c3ccc(N(c5ccccc5)c5ccc6c(c5)C(C)(C)c5ccc7ccccc7c5-6)cc3C4(C)C)cccc21] M232 | 1053 | 73.3% |
| 20 | I-1 | 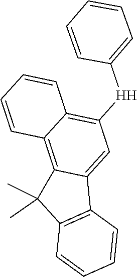 M246-1 | 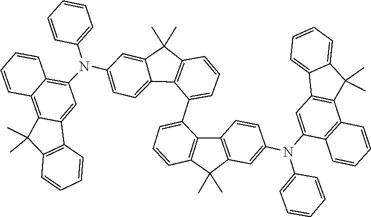 [SMILES: CC1(C)c2cc(N(c3ccccc3)c3cc4c(c5ccccc35)C(C)(C)c3ccccc3-4)ccc2-c2c(-c3cccc4c3-c3ccc(N(c5ccccc5)c5cc6c(c7ccccc57)C(C)(C)c5ccccc5-6)cc3C4(C)C)cccc21] M246 | 1053 | 69.8% |
| 21 | I-2 | 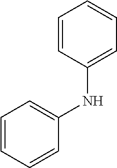 [SMILES: c1ccc(Nc2ccccc2)cc1] M1-1 | 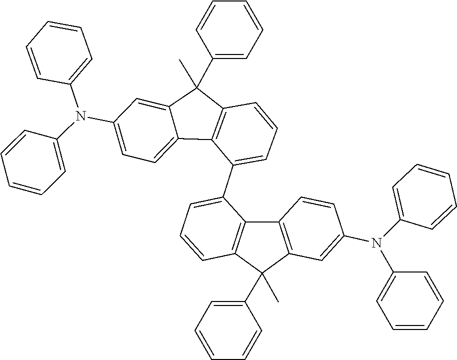 [SMILES: CC1(c2ccccc2)c2cc(N(c3ccccc3)c3ccccc3)ccc2-c2c(-c3cccc4c3-c3ccc(N(c5ccccc5)c5ccccc5)cc3C4(C)c3ccccc3)cccc21] M253 | 845 | 71.1% |
| 22 | I-3 | 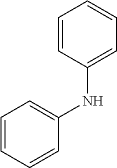 [SMILES: c1ccc(Nc2ccccc2)cc1] M1-1 | 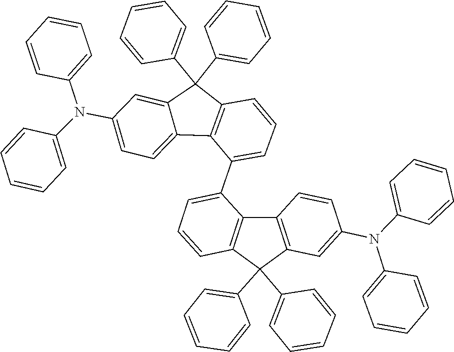 [SMILES: c1ccc(N(c2ccccc2)c2ccc3c(c2)C(c2ccccc2)(c2ccccc2)c2cccc(-c4cccc5c4-c4ccc(N(c6ccccc6)c6ccccc6)cc4C5(c4ccccc4)c4ccccc4)c2-3)cc1] M265 | 969 | 78.0% |
| 23 | I-4 | 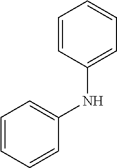 [SMILES: c1ccc(Nc2ccccc2)cc1] M1-1 | 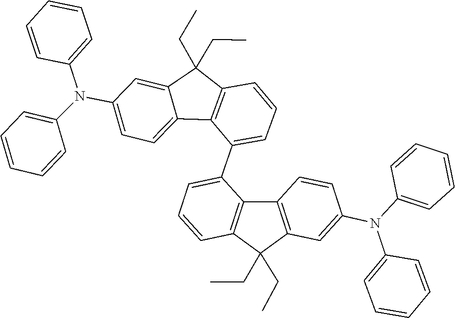 [SMILES: CCC1(CC)c2cc(N(c3ccccc3)c3ccccc3)ccc2-c2c(-c3cccc4c3-c3ccc(N(c5ccccc5)c5ccccc5)cc3C4(CC)CC)cccc21] M277 | 777 | 79.7% |
| 24 | I-1 | 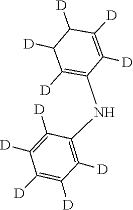 [SMILES: [2H]C1=C([2H])C([2H])C([2H])C([2H])=C1Nc1c([2H])c([2H])c([2H])c([2H])c1[2H]] M286-1 | 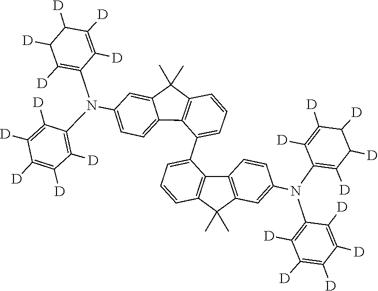 [SMILES: [2H]C1=C([2H])C([2H])C([2H])C([2H])=C1N(c1ccc2c(c1)C(C)(C)c1cccc(-c3cccc4c3-c3ccc(N(C5=C([2H])C([2H])C([2H])C([2H])=C5[2H])c5c([2H])c([2H])c([2H])c([2H])c5[2H])cc3C4(C)C)c1-2)c1c([2H])c([2H])c([2H])c([2H])c1[2H]] M286 | 745 | 67.9% |
| 25 | I-5 | 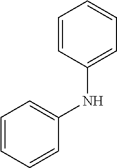 [SMILES: c1ccc(Nc2ccccc2)cc1] M1-1 | 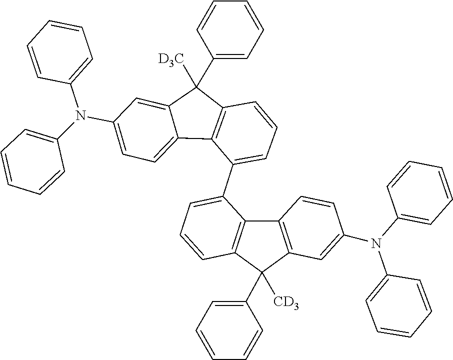 [SMILES: [2H]C([2H])([2H])C1(c2ccccc2)c2cc(N(c3ccccc3)c3ccccc3)ccc2-c2c(-c3cccc4c3-c3ccc(N(c5ccccc5)c5ccccc5)cc3C4(c3ccccc3)C([2H])([2H])[2H])cccc21] M290 | 851 | 81.2% |
| 26 | I-6 | 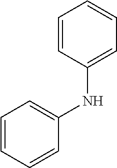 [SMILES: c1ccc(Nc2ccccc2)cc1] M1-1 | 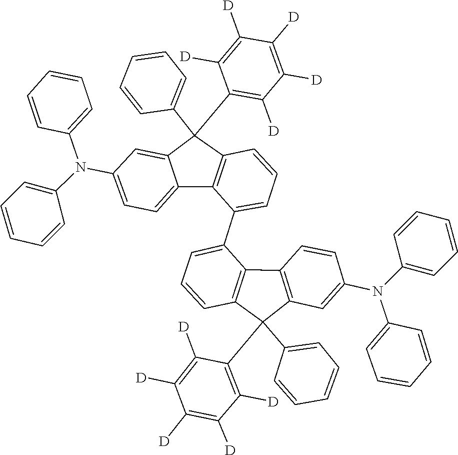 [SMILES: [2H]c1c([2H])c([2H])c(C2(c3ccccc3)c3cc(N(c4ccccc4)c4ccccc4)ccc3-c3c(-c4cccc5c4-c4ccc(N(c6ccccc6)c6ccccc6)cc4C5(c4ccccc4)c4c([2H])c([2H])c([2H])c([2H])c4[2H])cccc32)c([2H])c1[2H]] M295 | 979 | 70.3% |

### Example 27

### Synthesis of organic compound N27

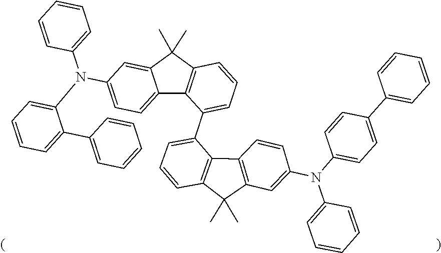 [SMILES: CC1(C)c2cc(N(c3ccccc3)c3ccc(-c4ccccc4)cc3)ccc2-c2c(-c3cccc4c3-c3ccc(N(c5ccccc5)c5ccccc5-c5ccccc5)cc3C4(C)C)cccc21]

Synthetic route of the organic compound N27 is as follows:

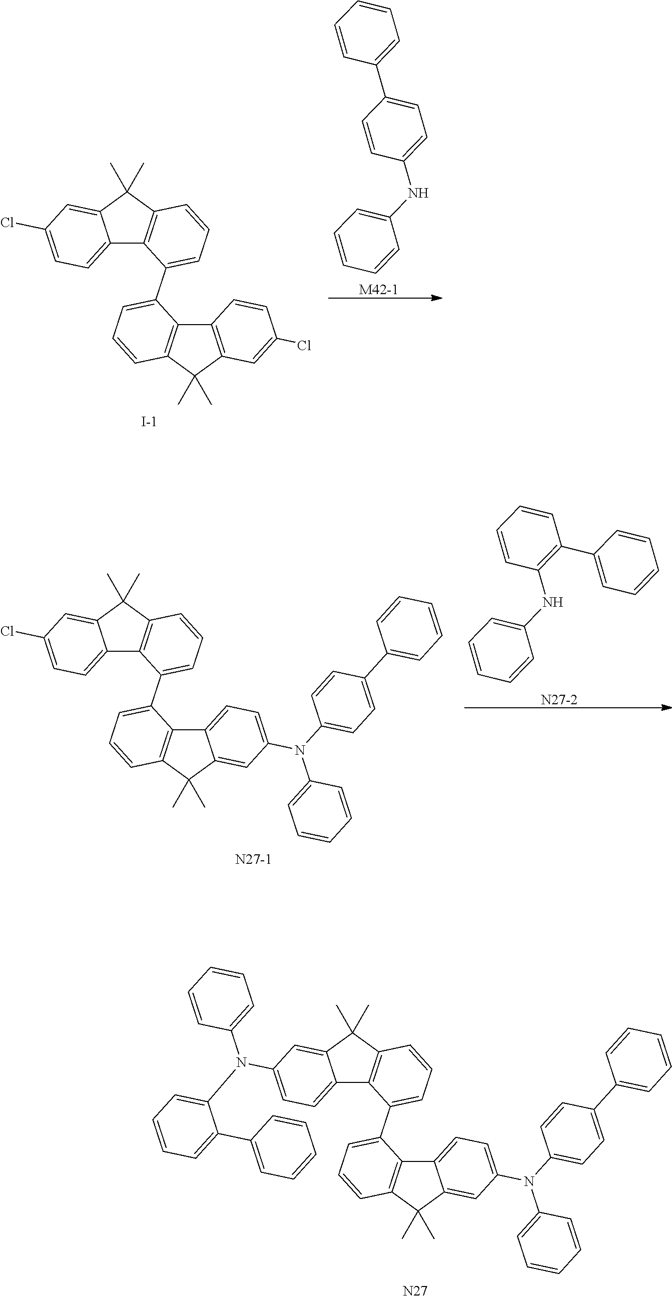

The specific synthetic steps of the organic compound N27 are as follows:

### Synthesis of Intermediate N27-1

I-1 (10.0 g, 0.02 mol) and M42-1 (5.4 g, 0.02 mol) were weighed and added into a clean three-necked flask, and Pd₂(dba)₃ (1%, 0.18 g), X-Phos (1.0 g, 2.0 mmol), sodium tert-butoxide (4.2 g, 0.04 mol), and anhydrous toluene (300 mL) were added therein. Air in the three-necked flask was displaced with nitrogen and the temperature is heated to 90° C. to reflux and react for 12 hours to obtain a mixture. After naturally cooling, the mixture was washed with water for separation, wherein the organic phase was dried and then spun to dryness, and purified through column chromatography to obtain the intermediate N27-1 with a yield of 56.0%. MS (ASAP)=664[M+H]⁺.

### Synthesis of Organic Compound N27

N27-1 (6.6 g, 0.01 mol) and N27-2 (2.5 g, 0.01 mol) were weighed and added into a clean three-necked flask, and Pd₂(dba)₃ (1%, 0.09 g), X-Phos (0.5 g, 1.0 mmol), sodium tert-butoxide (2.0 g, 0.02 mol), and anhydrous toluene (150 mL) were added therein. Air in the three-necked flask was displaced with nitrogen and the temperature is heated to 120° C. to reflux and react for 8 hours to obtain a mixture. After naturally cooling, the mixture was washed with water for separation, wherein the organic phase was dried and then spun to dryness, and purified through column chromatography to obtain the intermediate N27 with a yield of 76.8%. MS (ASAP)=873[M+H]⁺.

### Example 28

### Synthesis of Organic Compound N36

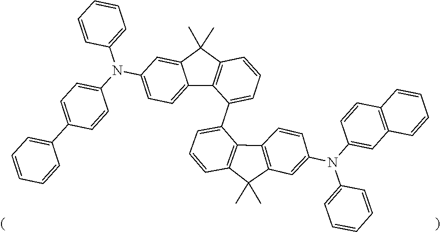 [SMILES: CC1(C)c2cc(N(c3ccccc3)c3ccc(-c4ccccc4)cc3)ccc2-c2c(-c3cccc4c3-c3ccc(N(c5ccccc5)c5ccc6ccccc6c5)cc3C4(C)C)cccc21]

Synthetic route of the organic compound N36 is as follows:

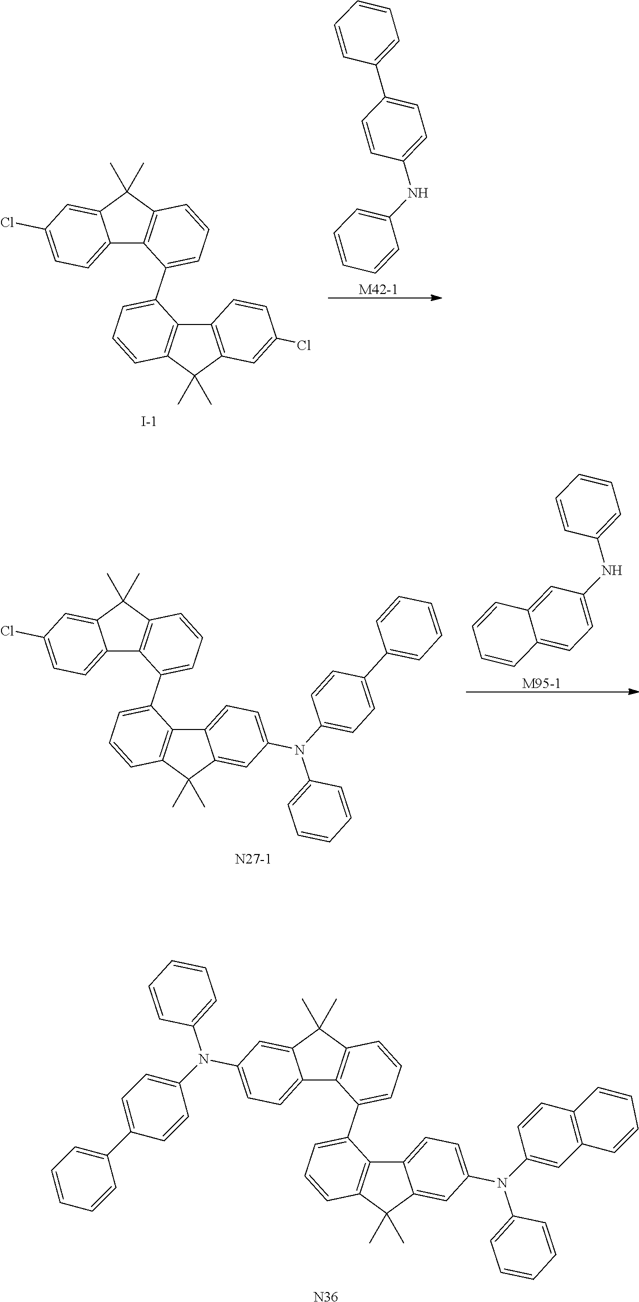

The specific synthetic steps of the organic compound N36 are as follows:

N27-1 (6.6 g, 0.01 mol) and M95-1 (2.2 g, 0.01 mol) were weighed and added into a clean three-necked flask, and Pd₂(dba)₃ (1%, 0.09 g), X-Phos (0.5 g, 1.0 mmol), sodium tert-butoxide (2.0 g, 0.02 mol), and anhydrous toluene (150 mL) were added therein. Air in the three-necked flask was displaced with nitrogen and the temperature is heated to 120° C. to reflux and react for 8 hours to obtain a mixture. After naturally cooling, the mixture was washed with water for separation, wherein the organic phase was dried and then spun to dryness, and purified through column chromatography to obtain the intermediate N36 with a yield of 81.0%. MS (ASAP)=847[M+H]⁺.

### Example 29

### Synthesis of Organic Compound N45

Synthetic route of organic compound N45 is as follows:

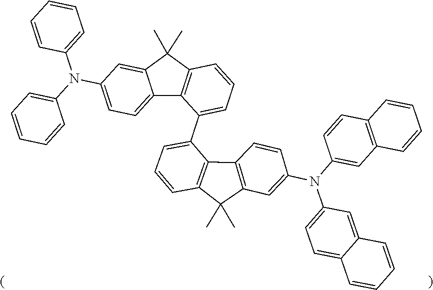 [SMILES: CC1(C)c2cc(N(c3ccccc3)c3ccccc3)ccc2-c2c(-c3cccc4c3-c3ccc(N(c5ccc6ccccc6c5)c5ccc6ccccc6c5)cc3C4(C)C)cccc21]

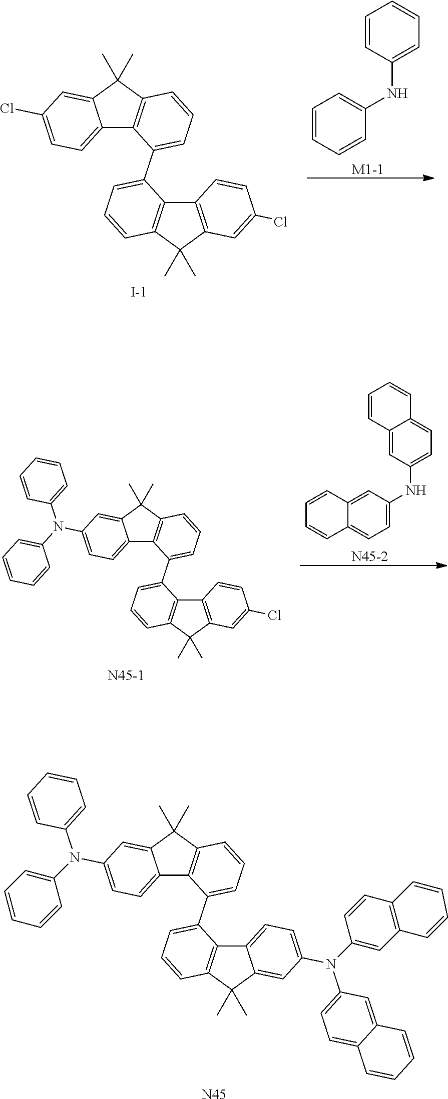

The specific synthetic steps of organic compound N45 are as follows:

### Synthesis of Intermediate N45-1

I-1 (10.0 g, 0.02 mol) and M1-1 (3.7 g, 0.02 mol) were weighed and added into a clean three-necked flask, and Pd₂(dba)₃ (1%, 0.18 g), X-Phos (1.0 g, 2.0 mmol), sodium tert-butoxide (4.2 g, 0.04 mol), and anhydrous toluene (300 mL) were added therein. Air in the three-necked flask was displaced with nitrogen and the temperature is heated to 90° C. to reflux and react for 12 hours to obtain a mixture. After naturally cooling, the mixture was washed with water for separation, wherein the organic phase was dried and then spun to dryness, and purified through column chromatography to obtain the intermediate N27-1 with a yield of 60.2%. MS (ASAP)=588[M+H]⁺.

### Synthesis of Organic Compound N45

N45-1 (6.0 g, 0.01 mol) and N45-2 (2.7 g, 0.01 mol) were weighed and added into a clean three-necked flask, and Pd₂(dba)₃ (1%, 0.09 g), X-Phos (0.5 g, 1.0 mmol), sodium tert-butoxide (2.0 g, 0.02 mol), and anhydrous toluene (150 mL) were added therein. Air in the three-necked flask was displaced with nitrogen and the temperature is heated to 120° C. to reflux and react for 8 hours to obtain a mixture. After naturally cooling, the mixture was washed with water for separation, wherein the organic phase was dried and then spun to dryness, and purified through column chromatography to obtain the intermediate N45 with a yield of 79.5%. MS (ASAP)=821[M+H]⁺.

### Example 30

### Synthesis of Organic Compound N75

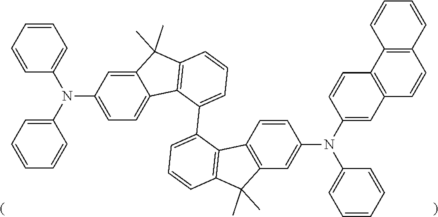 [SMILES: CC1(C)c2cc(N(c3ccccc3)c3ccccc3)ccc2-c2c(-c3cccc4c3-c3ccc(N(c5ccccc5)c5ccc6c(ccc7ccccc76)c5)cc3C4(C)C)cccc21]

Synthetic route of organic compound N75 is as follows:

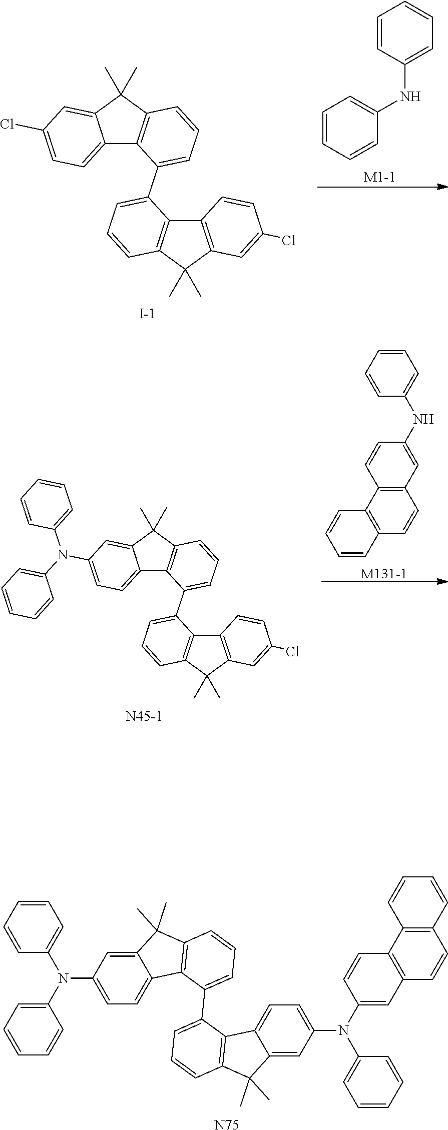

The specific synthetic steps of the organic compound N75 are as follows:

N45-1 (6.0 g, 0.01 mol) and M131-1 (2.7 g, 0.01 mol) were weighed and added into a clean three-necked flask, and Pd₂(dba)₃ (1%, 0.09 g), X-Phos (0.5 g, 1.0 mmol), sodium tert-butoxide (2.0 g, 0.02 mol), and anhydrous toluene (150 mL) were added therein. Air in the three-necked flask was displaced with nitrogen and the temperature is heated to 120° C. to reflux and react for 8 hours to obtain a mixture. After naturally cooling, the mixture was washed with water for separation, wherein the organic phase was dried and then spun to dryness, and purified through column chromatography to obtain the intermediate N75 with a yield of 78.0%. MS (ASAP)=821[M+H]⁺.

### Example 31

### Synthesis of Organic Compound N95

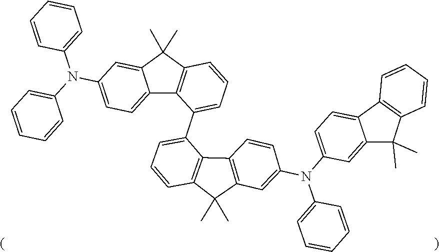 [SMILES: CC1(C)c2ccccc2-c2ccc(N(c3ccccc3)c3ccc4c(c3)C(C)(C)c3cccc(-c5cccc6c5-c5ccc(N(c7ccccc7)c7ccccc7)cc5C6(C)C)c3-4)cc21]

Synthetic route of the organic compound N95 is as follows:

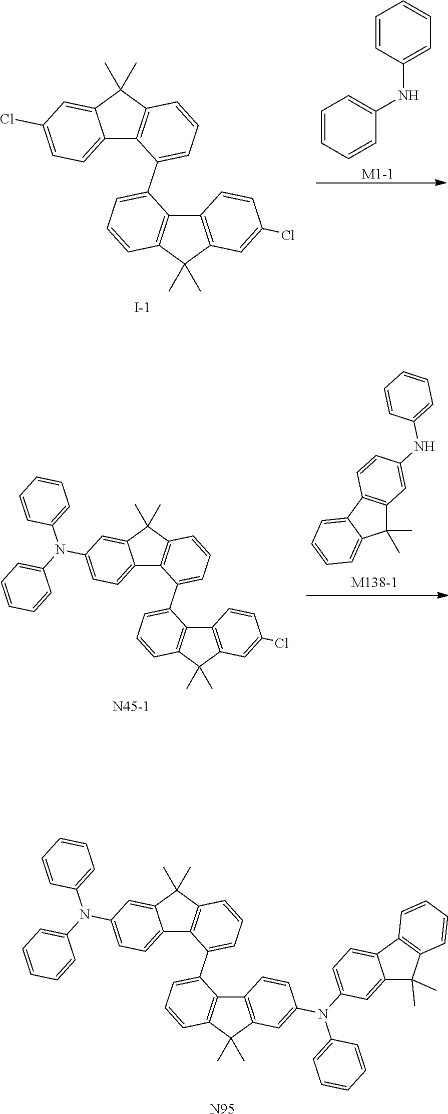

The specific synthetic steps of the organic compound N95 are as follows:

N45-1 (6.0 g, 0.01 mol) and M138-1 (2.9 g, 0.01 mol) were weighed and added into a clean three-necked flask, and Pd₂(dba)₃ (1%, 0.09 g), X-Phos (0.5 g, 1.0 mmol), sodium tert-butoxide (2.0 g, 0.02 mol), and anhydrous toluene (150 mL) were added therein. Air in the three-necked flask was displaced with nitrogen and the temperature is heated to 120° C. to reflux and react for 8 hours to obtain a mixture. After naturally cooling, the mixture was washed with water for separation, wherein the organic phase was dried and then spun to dryness, and purified through column chromatography to obtain the intermediate N95 with a yield of 70.3%. MS (ASAP)=837[M+H]⁺.

### Example 32

### Synthesis of Organic Compound N126

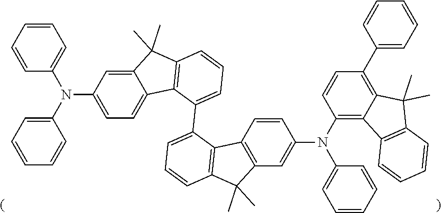 [SMILES: CC1(C)c2cc(N(c3ccccc3)c3ccccc3)ccc2-c2c(-c3cccc4c3-c3ccc(N(c5ccccc5)c5ccc(-c6ccccc6)c6c5-c5ccccc5C6(C)C)cc3C4(C)C)cccc21]

The synthetic route of the organic compound N126 is as follows:

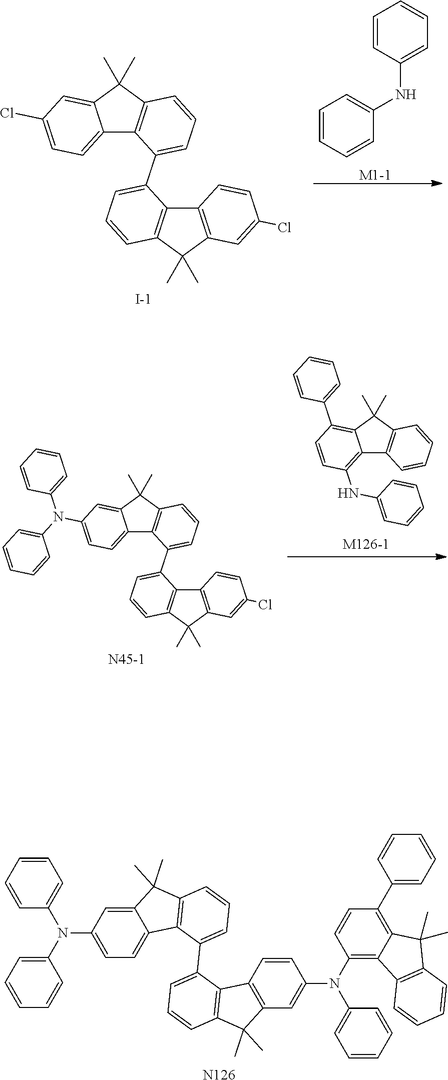

The specific synthetic steps of the organic compound N126 are as follows:

N45-1 (6.0 g, 0.01 mol) and N126-1 (3.6 g, 0.01 mol) were weighed and added into a clean three-necked flask, and Pd₂(dba)₃ (1%, 0.09 g), X-Phos (0.5 g, 1.0 mmol), sodium tert-butoxide (2.0 g, 0.02 mol), and anhydrous toluene (150 mL) were added therein. Air in the three-necked flask was displaced with nitrogen and the temperature is heated to 120° C. to reflux and react for 8 hours to obtain a mixture. After naturally cooling, the mixture was washed with water for separation, wherein the organic phase was dried and then spun to dryness, and purified through column chromatography to obtain the intermediate N126 with a yield of 65.2%. MS (ASAP)=913[M+H]⁺.

### Example 33

### Synthesis of Organic Compound N173

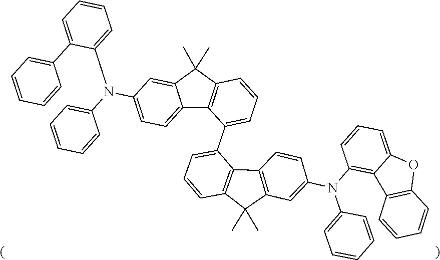 [SMILES: CC1(C)c2cc(N(c3ccccc3)c3ccccc3-c3ccccc3)ccc2-c2c(-c3cccc4c3-c3ccc(N(c5ccccc5)c5cccc6oc7ccccc7c56)cc3C4(C)C)cccc21]

The synthetic route of the organic compound N173 is as follows:

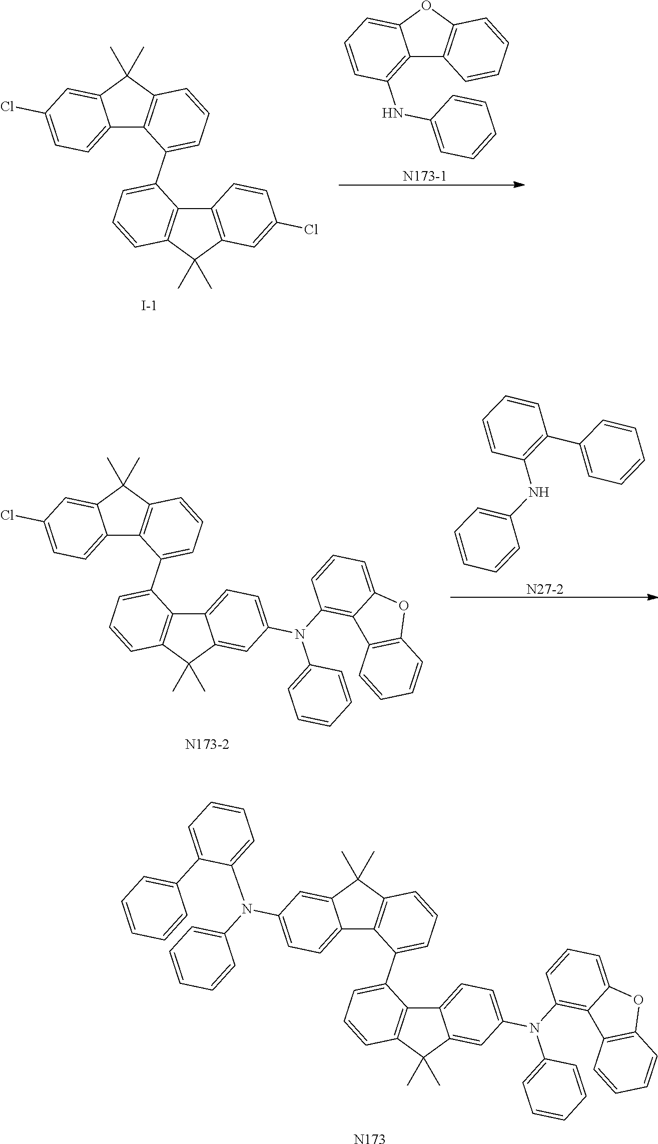

The specific synthetic steps of the organic compound N173 are as follows:

### Synthesis of Intermediate N173-2

I-1 (10.0 g, 0.02 mol) and N173-1 (5.4 g, 0.02 mol) were weighed and added into a clean three-necked flask, and Pd₂(dba)₃ (1%, 0.18 g), X-Phos (1.0 g, 2.0 mmol), sodium tert-butoxide (4.2 g, 0.04 mol), and anhydrous toluene (300 mL) were added therein. Air in the three-necked flask was displaced with nitrogen and the temperature is heated to 90° C. to reflux and react for 12 hours to obtain a mixture. After naturally cooling, the mixture was washed with water for separation, wherein the organic phase was dried and then spun to dryness, and purified through column chromatography to obtain the intermediate N173-2 with a yield of 55.5%. MS (ASAP)=678[M+H]⁺.

### Synthesis of Organic Compound N173

N173-2 (6.8 g, 0.01 mol) and N27-2 (2.5 g, 0.01 mol) were weighed and added into a clean three-necked flask, and Pd₂(dba)₃ (1%, 0.09 g), X-Phos (0.5 g, 1.0 mmol), sodium tert-butoxide (2.0 g, 0.02 mol), and anhydrous toluene (150 mL) were added therein. Air in the three-necked flask was displaced with nitrogen and the temperature is heated to 120° C. to reflux and react for 8 hours to obtain a mixture. After naturally cooling, the mixture was washed with water for separation, wherein the organic phase was dried and then spun to dryness, and purified through column chromatography to obtain the intermediate N173 with a yield of 68.9%. MS (ASAP)=887[M+H]⁺.

### Example 34

### Synthesis of Organic Compound N236

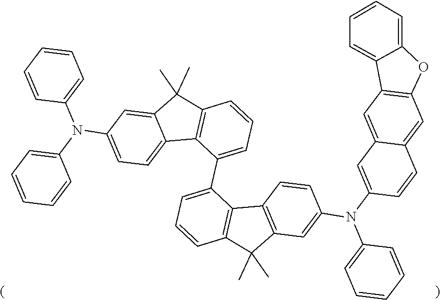 [SMILES: CC1(C)c2cc(N(c3ccccc3)c3ccccc3)ccc2-c2c(-c3cccc4c3-c3ccc(N(c5ccccc5)c5ccc6cc7oc8ccccc8c7cc6c5)cc3C4(C)C)cccc21]

The synthetic route of the organic compound N236 is as follows:

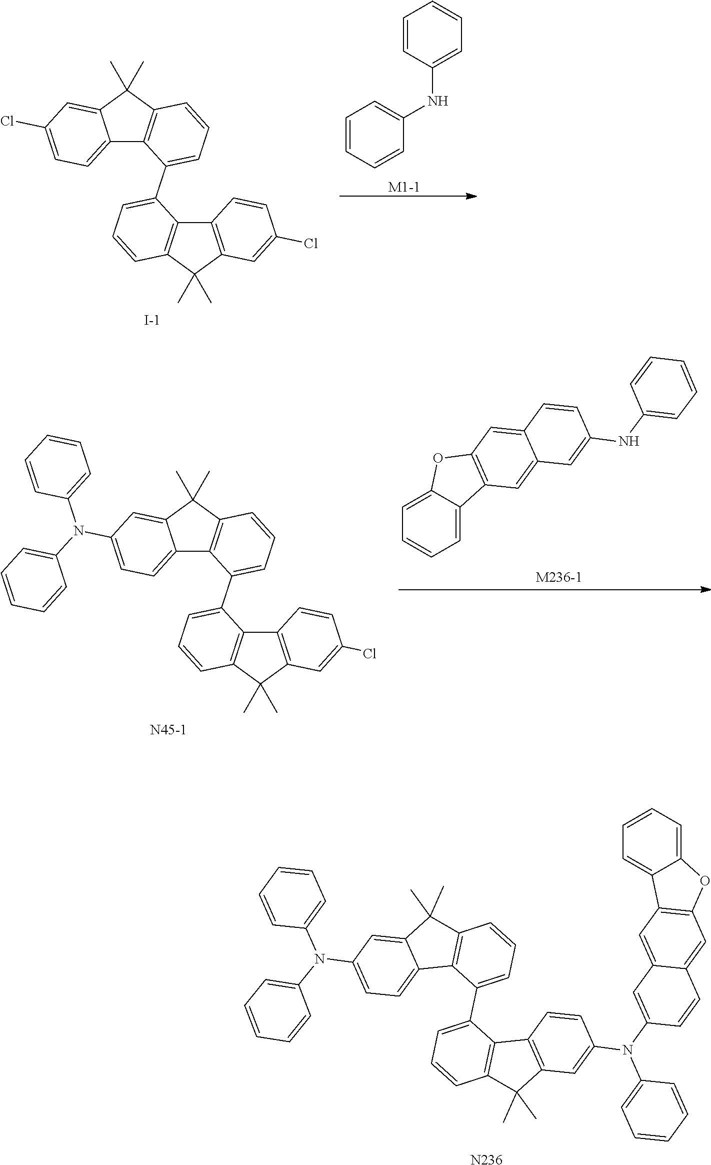

The specific synthetic steps of the organic compound N236 are as follows:

N45-1 (6.0 g, 0.01 mol) and N236-1 (3.1 g, 0.01 mol) were weighed and added into a clean three-necked flask, and Pd₂(dba)₃ (1%, 0.09 g), X-Phos (0.5 g, 1.0 mmol), sodium tert-butoxide (2.0 g, 0.02 mol), and anhydrous toluene (150 mL) were added therein. Air in the three-necked flask was displaced with nitrogen and the temperature is heated to 120° C. to reflux and react for 8 hours to obtain a mixture. After naturally cooling, the mixture was washed with water for separation, wherein the organic phase was dried and then spun to dryness, and purified through column chromatography to obtain the intermediate N236 with a yield of 76.7%. MS (ASAP)=861[M+H]⁺.

### Example 35

### Synthesis of Organic Compound N262

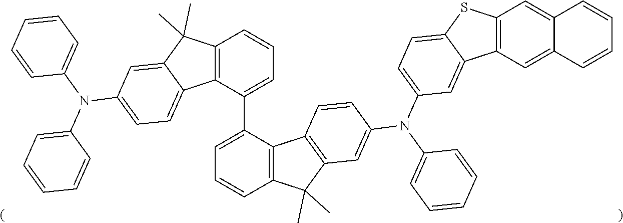 [SMILES: CC1(C)c2cc(N(c3ccccc3)c3ccccc3)ccc2-c2c(-c3cccc4c3-c3ccc(N(c5ccccc5)c5ccc6sc7cc8ccccc8cc7c6c5)cc3C4(C)C)cccc21]

The synthetic route of the organic compound N262 is as follows:

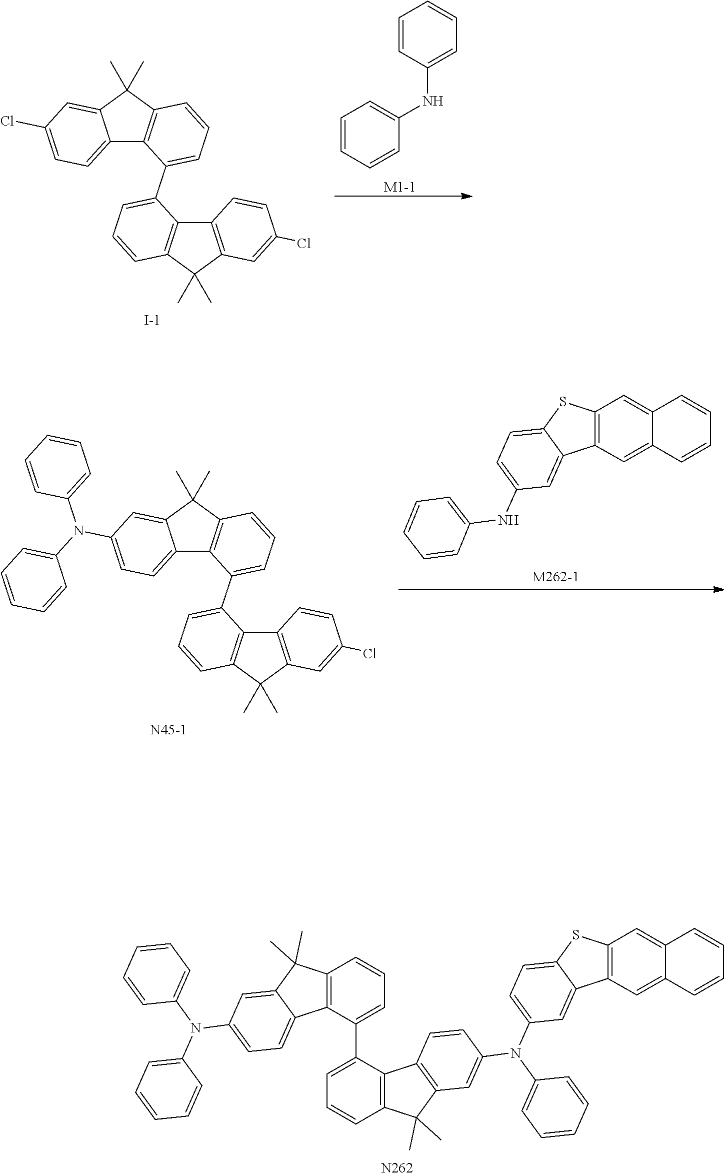

The specific synthetic steps of the organic compound N262 are as follows:

N45-1 (6.0 g, 0.01 mol) and N262-1 (3.3 g, 0.01 mol) were weighed and added into a clean three-necked flask, and Pd₂(dba)₃ (1%, 0.09 g), X-Phos (0.5 g, 1.0 mmol), sodium tert-butoxide (2.0 g, 0.02 mol), and anhydrous toluene (150 mL) were added therein. Air in the three-necked flask was displaced with nitrogen and the temperature is heated to 120° C. to reflux and react for 8 hours to obtain a mixture. After naturally cooling, the mixture was washed with water for separation, wherein the organic phase was dried and then spun to dryness, and purified through column chromatography to obtain the intermediate N262 with a yield of 71.3%. MS (ASAP)=877[M+H]⁺.

### Example 36

### Synthesis of Organic Compound N294

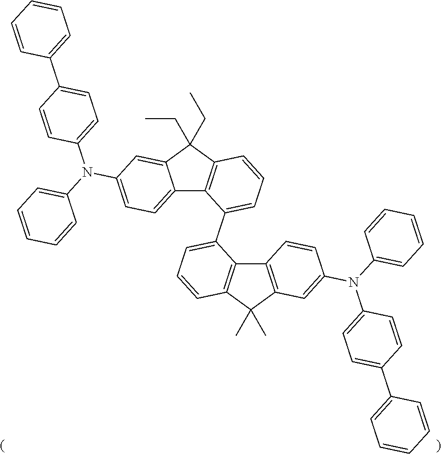 [SMILES: CCC1(CC)c2cc(N(c3ccccc3)c3ccc(-c4ccccc4)cc3)ccc2-c2c(-c3cccc4c3-c3ccc(N(c5ccccc5)c5ccc(-c6ccccc6)cc5)cc3C4(C)C)cccc21]

The synthetic route of the organic compound N294 is as follows:

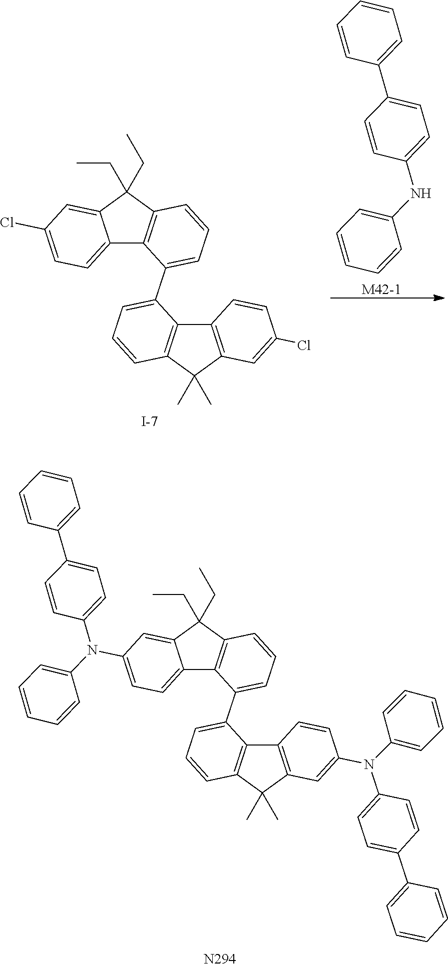

The specific synthetic steps of the organic compound N294 are as follows:

I-7 (9.6 g, 0.02 mol) and M42-1 (12.3 g, 0.05 mol) were weighed and added into a clean three-necked flask, and Pd₂(dba)₃ (1%, 0.18 g), X-Phos (1.0 g, 2.0 mmol), sodium tert-butoxide (5.8 g, 0.06 mol), and anhydrous toluene (300 mL) were added therein. Air in the three-necked flask was displaced with nitrogen and the temperature is heated to 120° C. to reflux and react for 12 hours to obtain a mixture. After naturally cooling, the mixture was washed with water for separation, wherein the organic phase was dried and then spun to dryness, and purified through column chromatography to obtain the intermediate N294 with a yield of 80.8%. MS (ASAP)=901[M+H]⁺.

### Example 37

### Synthesis of Organic Compound N318

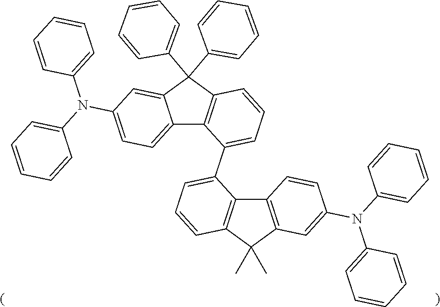 [SMILES: CC1(C)c2cc(N(c3ccccc3)c3ccccc3)ccc2-c2c(-c3cccc4c3-c3ccc(N(c5ccccc5)c5ccccc5)cc3C4(c3ccccc3)c3ccccc3)cccc21]

The synthetic route of the organic compound N318 is as follows:

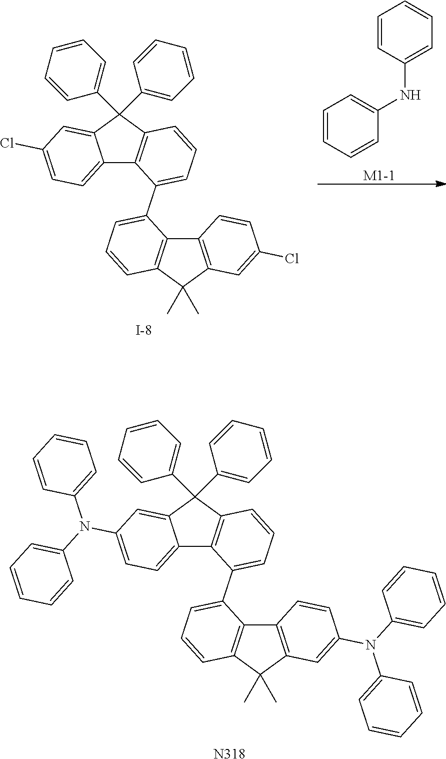

The specific synthetic steps of the organic compound N318 are as follows:

I-8 (11.6 g, 0.02 mol) and M1-1 (8.5 g, 0.05 mol) were weighed and added into a clean three-necked flask, and Pd₂(dba)₃ (1%, 0.18 g), X-Phos (1.0 g, 2.0 mmol), sodium tert-butoxide (5.8 g, 0.06 mol), and anhydrous toluene (300 mL) were added therein. Air in the three-necked flask was displaced with nitrogen and the temperature is heated to 120° C. to reflux and react for 12 hours to obtain a mixture. After naturally cooling, the mixture was washed with water for separation, wherein the organic phase was dried and then spun to dryness, and purified through column chromatography to obtain the intermediate N318 with a yield of 81.6%. MS (ASAP)=845[M+H]⁺.

### Example 38

### Synthesis of Organic Compound N341

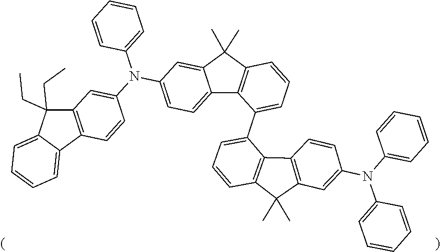 [SMILES: CCC1(CC)c2ccccc2-c2ccc(N(c3ccccc3)c3ccc4c(c3)C(C)(C)c3cccc(-c5cccc6c5-c5ccc(N(c7ccccc7)c7ccccc7)cc5C6(C)C)c3-4)cc21]

The synthetic route of the organic compound N341 is as follows:

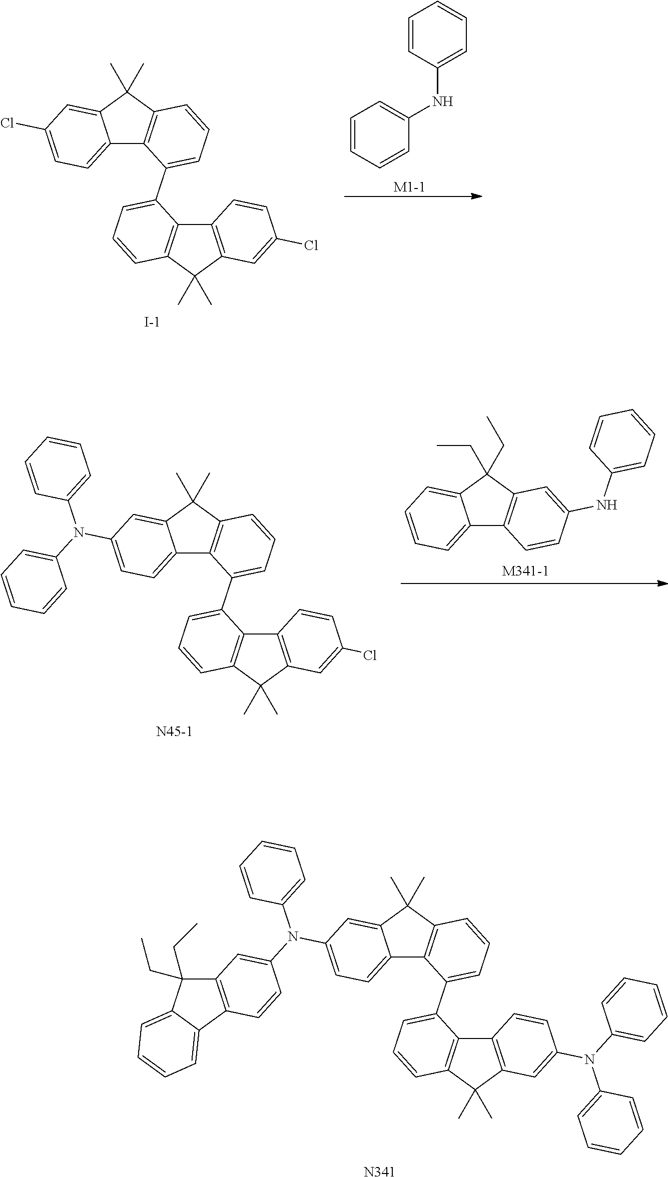

The specific synthetic steps of the organic compound N341 are as follows:

N45-1 (6.0 g, 0.01 mol) and N345-1 (3.1 g, 0.01 mol) were weighed and added into a clean three-necked flask, and Pd₂(dba)₃ (1%, 0.09 g), X-Phos (0.5 g, 1.0 mmol), sodium tert-butoxide (2.0 g, 0.02 mol), and anhydrous toluene (150 mL) were added therein. Air in the three-necked flask was displaced with nitrogen and the temperature is heated to 120° C. to reflux and react for 8 hours to obtain a mixture. After naturally cooling, the mixture was washed with water for separation, wherein the organic phase was dried and then spun to dryness, and purified through column chromatography to obtain the intermediate N341 with a yield of 80.8%. MS (ASAP)=865[M+H]⁺.

### Example 39

### Synthesis of Organic Compound N345

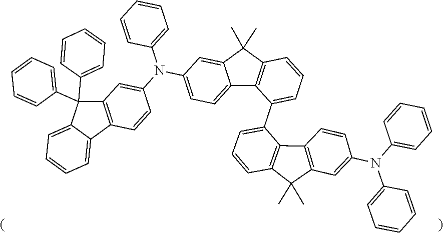 [SMILES: CC1(C)c2cc(N(c3ccccc3)c3ccccc3)ccc2-c2c(-c3cccc4c3-c3ccc(N(c5ccccc5)c5ccc6c(c5)C(c5ccccc5)(c5ccccc5)c5ccccc5-6)cc3C4(C)C)cccc21]

The synthetic route of organic compound N345 is as follows:

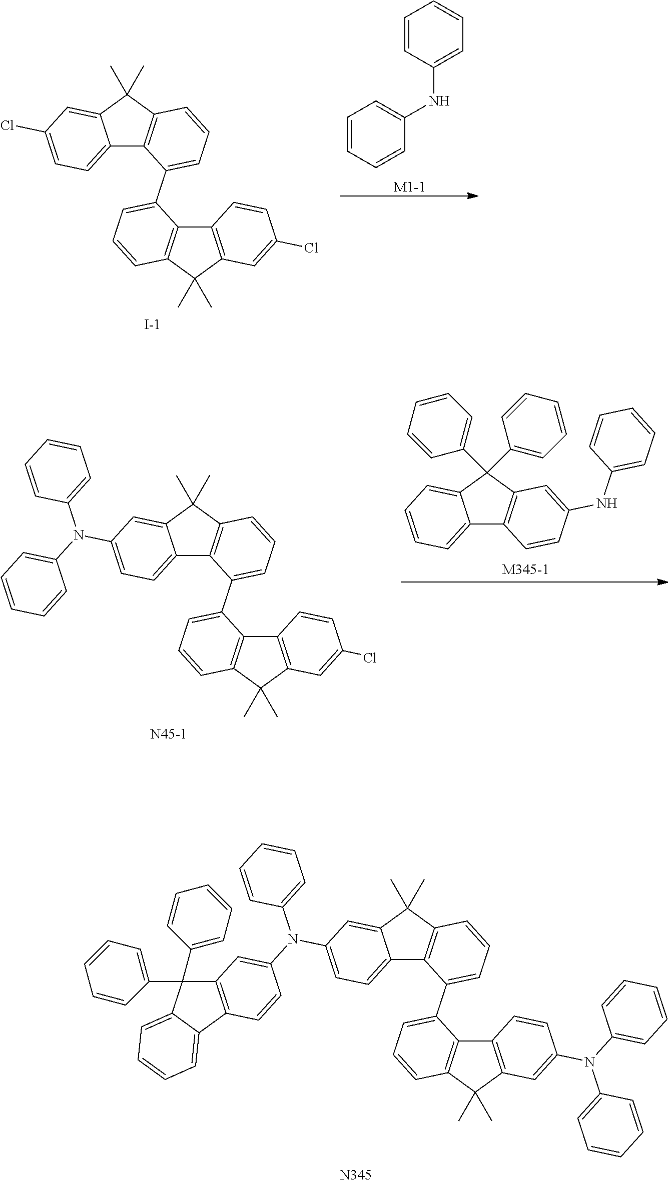

The specific synthetic steps of the organic compound N345 are as follows:

N45-1 (6.0 g, 0.01 mol) and N345-1 (4.1 g, 0.01 mol) were weighed and added into a clean three-necked flask, and Pd₂(dba)₃ (1%, 0.09 g), X-Phos (0.5 g, 1.0 mmol), sodium tert-butoxide (2.0 g, 0.02 mol), and anhydrous toluene (150 mL) were added therein. Air in the three-necked flask was displaced with nitrogen and the temperature is heated to 120° C. to reflux and react for 8 hours to obtain a mixture. After naturally cooling, the mixture was washed with water for separation, wherein the organic phase was dried and then spun to dryness, and purified through column chromatography to obtain the intermediate N345 with a yield of 79.8%. MS (ASAP)=961[M+H]⁺.

### Comparative Example

Comparative compound 1, comparative compound 2, comparative compound 3, and comparative compound 4 are used as comparative examples of the above-mentioned examples 1 to 39. Comparative compound 1, comparative compound 2, comparative compound 3, and comparative compound 4 are recorded as “Ref 1”, “Ref 2”, “Ref 3” and “Ref 4” respectively, and their structural formulas are as follows:

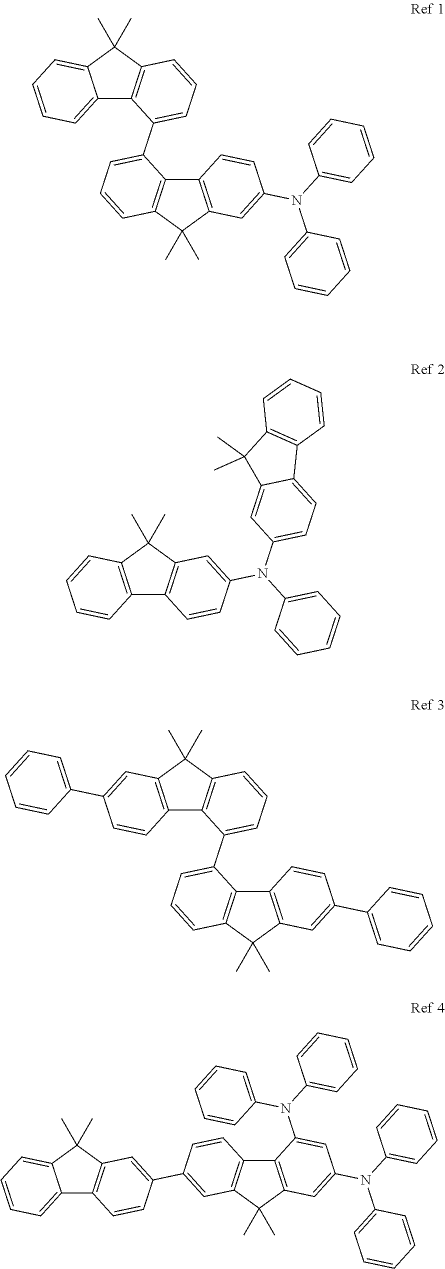

As shown in Table 3, the HOMO level, LUMO level, E_{T1} (first excited triplet state) level, and E_{S1} (first excited singlet state) level of the organic compounds obtained in Examples 1 to 39 and Comparative Compounds 1 to 4 can be obtained by quantum calculation. Specifically, they are calculated by using TD-DFT (time-dependent density functional theory) through Gaussian09W (Gaussian Inc.), specifically, the software for calculating the above-mentioned energy levels is Gaussian 09W (Gaussian Inc.). The specific simulation method can be found in patent document WO2011141110A2. First, an optimized ground state molecular structure is calculated under the B3LYP/6-31G (d) basis set according to the density functional theory, and then, E_{S1} and E_{T1}, HOMO and LUMO energy levels are calculated under the B3PW91/6-31G (d) basis set according to the optimized ground state structure by using the time-dependent density functional theory, and the E_{S1} and E_{T1} are directly used.

$$HOMO ( eV ) = ( ( HOMO ( G ) \times 27.212 ) - 0.9899 ) / 1.1206$$

$$LUMO ( eV ) = ( ( LUMO ( G ) \times 27.212 ) - 2.0041 ) / 1.385$$

where HOMO(G) and LUMO(G) are direct calculation results of Gaussian09W in Hartree.

TABLE 3
Calculation results of HOMO levels, LUMO levels,
E_{S1} levels and E_{T1} Levels of organic compounds and
comparative compounds 1 to comparative compound 4

| Compound | HOMO [eV] | LUMO [eV] | ES1 [eV] | ET1 [eV] |
|---|---|---|---|---|
| M1 | −5.25 | −2.12 | 3.48 | 2.66 |
| M5 | −5.12 | −2.06 | 3.42 | 2.64 |
| M24 | −5.18 | −2.09 | 3.45 | 2.65 |
| M34 | −5.17 | −2.08 | 3.45 | 2.65 |
| M42 | −5.24 | −2.19 | 3.36 | 2.63 |
| M73 | −5.22 | −2.15 | 3.40 | 2.64 |
| M91 | −5.23 | −2.18 | 3.34 | 2.63 |
| M95 | −5.24 | −2.24 | 3.27 | 2.46 |
| M121 | −5.25 | −2.37 | 3.04 | 2.39 |
| M131 | −5.26 | −2.24 | 3.28 | 2.54 |
| M138 | −5.17 | −2.19 | 3.29 | 2.57 |
| M158 | −5.21 | −2.24 | 3.27 | 2.59 |
| M171 | −5.26 | −2.26 | 3.27 | 2.65 |
| M187 | −5.24 | −2.29 | 3.23 | 2.62 |
| M222 | −5.20 | −2.40 | 3.12 | 2.34 |
| M253 | −5.27 | −2.19 | 3.41 | 2.64 |
| M265 | −5.28 | −2.22 | 3.41 | 2.62 |
| M277 | −5.24 | −2.11 | 3.48 | 2.66 |
| N27 | −5.27 | −2.19 | 3.40 | 2.64 |
| N36 | −5.25 | −2.25 | 3.29 | 2.46 |
| N45 | −5.23 | −2.36 | 3.13 | 2.36 |
| N75 | −5.25 | −2.24 | 3.27 | 2.54 |
| N95 | −5.18 | −2.19 | 3.30 | 2.57 |
| N126 | −5.23 | −2.17 | 3.33 | 2.67 |
| Ref 1 | −5.25 | −2.11 | 3.52 | 2.66 |
| Ref 2 | −5.21 | −2.20 | 3.34 | 2.57 |
| Ref 3 | −5.91 | −2.29 | 4.02 | 2.78 |
| Ref 4 | −5.28 | −2.42 | 3.18 | 2.44 |

It can be seen from Table 3 that the HOMO levels of the organic compounds provided in the embodiments of the present disclosure range from −5.1 eV to −5.3 eV, which can meet the energy level barrier requirements of different light-emitting elements, further, the first excited state level is greater than 3.0 eV, thereby avoiding interference with the light color of visible light emitted from the light-emitting layer of the light-emitting element.

In addition, referring to FIG. 2, the optimized molecular structure and HOMO level distribution of the organic compound M95 provided in the present disclosure are shown in the figure. The molecular orbital distribution of the organic compound M95 provided in the present disclosure is uniform, and two connected fluorenyl groups are connected by a single bond to form a nearly orthogonal molecular conformation, which provides stable and good hole transport performance and is beneficial for use as hole transport materials in light-emitting elements, thereby facilitating application as a hole transport material in a light-emitting element.

Exemplary manufacturing steps of the light-emitting elements provided by the present disclosure are shown in the following exemplary example 40.

### Example 40

The light-emitting element according to this example includes an anode layer, a hole injection layer, a hole transport layer, a light-emitting auxiliary layer, an organic light-emitting layer, an electron transport layer, an electron injection layer, and a cathode layer that are sequentially formed on a substrate. The specific manufacturing steps are as follows:
a. Cleaning of the ITO anode: ultrasonic cleaning with deionized water, acetone, and isopropanol for 15 minutes, followed by treating in a plasma cleaner for 5 minutes to improve the work function of the electrode;
b. Forming a hole injection layer by subjecting a hole injection layer material HATCN to evaporation through vacuum evaporation on the ITO anode at an evaporation rate of 1 Å/s, which has a thickness of 30 nm;
c. Forming a hole transport layer by subjecting a hole transport material to evaporation through vacuum evaporation on the hole injection layer at an evaporation rate of 1 Å/s, which has a thickness of 60 nm;
d. Forming a light-emitting auxiliary layer by subjecting a light-emitting auxiliary material Prime to evaporation on the hole transport layer at an evaporation rate of 1 Å/s, which has a thickness of 10 nm;
e. Forming a light-emitting layer by subjecting a light-emitting layer to evaporation on the light-emitting auxiliary layer at an evaporation rate of 1 Å/s, wherein BH is used as a host material, BD is used as a doping material, the mass ratio of BH to BD is 98:2, and the thickness of the light-emitting layer is 25 nm;
f. Forming an electron transport layer by placing electron transport materials ET and Liq in different evaporation crucibles in a vacuum chamber, and co-depositing ET and Liq at a weight ratio of 5:5 in a high vacuum environment (1×10⁻⁶ mbar), which has a thickness of 30 nm;
g. Forming an electron injection layer by subjecting an electron injection material LiQ to vacuum evaporation on the electron transport layer at an evaporation rate of 1 Å/s, which has a thickness of 1 nm;
h. Forming a cathode layer: subjecting Al to vacuum evaporation on the electron injection layer at an evaporation rate of 1 Å/s to obtain an Al cathode, which has a thickness of 100 nm;
i. Encapsulation: The device is encapsulated with UV-curable resin in a nitrogen glove box.

Specifically, in this example, the light-emitting element 1 to the light-emitting element 39 and the comparative element 1 to the comparative element 4 are obtained by the above steps. The hole transport materials used in the light-emitting elements 1 to 39 are organic compounds obtained in examples 1 to 39, respectively, and the hole transport materials used in the comparative elements 1 to 4 are Ref 1, Ref 2, Ref 3 and Ref 4, respectively.

Specifically, HATCN, Prime, BH, BD, ET, Liq, Ref 1, Ref 2, Ref 3 and Ref 4 are commercially available or synthesized from known synthesis methods and existing raw materials, wherein the chemical structures of Ref 1, Ref 2, Ref 3 and Ref 4 are as shown above, and the chemical structures of other compounds are as follows:

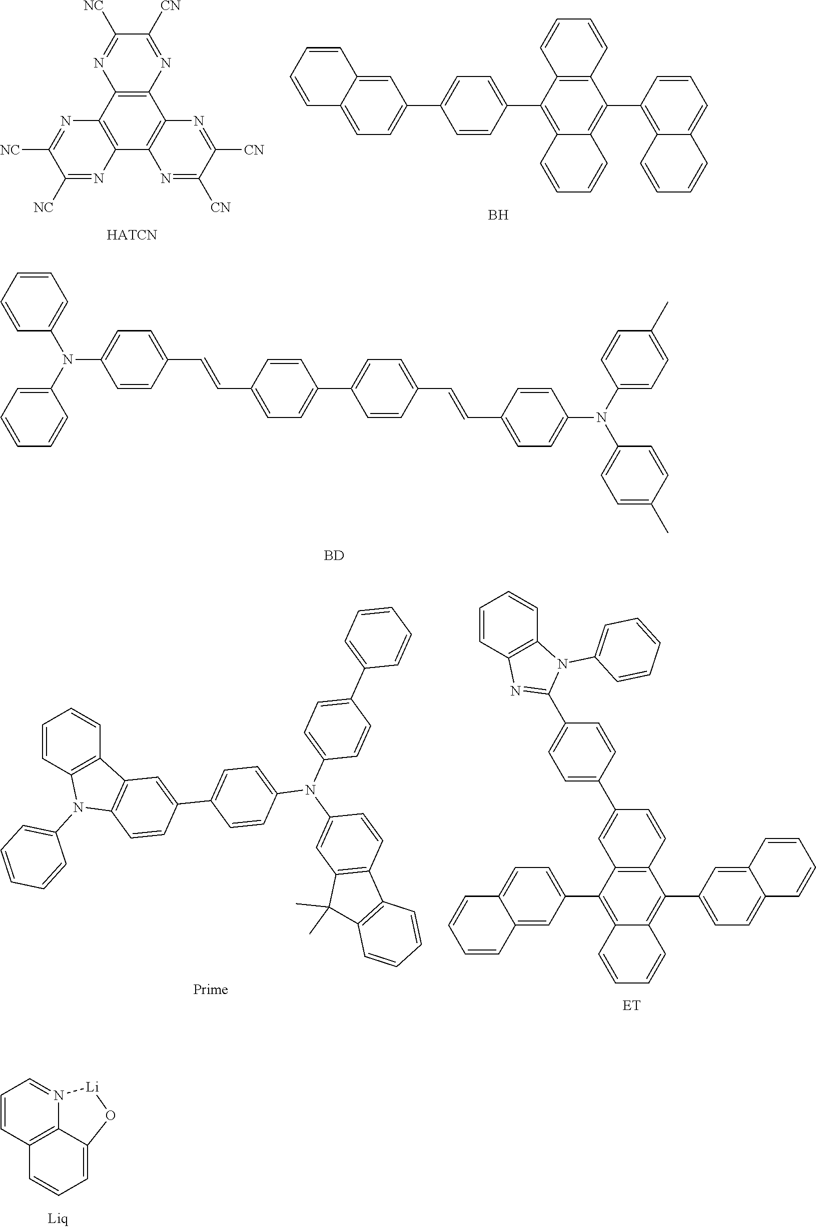

In this example, the current-voltage (J-V) characteristics of the light-emitting element 1 to the light-emitting element 39 and the comparative element 1 to the comparative element 4 are tested. The voltage of each light-emitting element and the comparative element at a current density of 10 mA/cm², the time required (relative lifetime) for the brightness to drop to 90% of initial brightness @1000 nits under constant current, and the relative luminous efficiency are obtained. Specific results are shown in Table 1.

TABLE 4
Performance data of light-emitting element 1 to light-emitting
element 39 and comparative element 1 to comparative element 4

| | Hole trans- | | Relative | |
|---|---|---|---|---|
| | port layer | | luminous | Relative |
| | material | Voltage | efficiency | lifetime |
| Light-emitting element 1 | M1 | 4.6 | 146% | 139% |
| Light-emitting element 2 | M5 | 4.4 | 130% | 130% |
| Light-emitting element 3 | M24 | 4.4 | 128% | 127% |
| Light-emitting element 4 | M34 | 4.4 | 126% | 136% |
| Light-emitting element 5 | M39 | 4.4 | 132% | 141% |
| Light-emitting element 6 | M42 | 4.3 | 168% | 153% |
| Light-emitting element 7 | M73 | 4.4 | 155% | 133% |
| Light-emitting element 8 | M91 | 4.4 | 157% | 138% |
| Light-emitting element 9 | M95 | 4.4 | 165% | 146% |
| Light-emitting element 10 | M121 | 4.4 | 144% | 148% |
| Light-emitting element 11 | M124 | 4.4 | 172% | 144% |
| Light-emitting element 12 | M131 | 4.5 | 158% | 141% |
| Light-emitting element 13 | M138 | 4.4 | 177% | 148% |
| Light-emitting element 14 | M158 | 4.4 | 127% | 120% |
| Light-emitting element 15 | M171 | 4.4 | 120% | 118% |
| Light-emitting element 16 | M187 | 4.4 | 130% | 144% |
| Light-emitting element 17 | M211 | 4.3 | 143% | 150% |
| Light-emitting element 18 | M222 | 4.4 | 160% | 142% |
| Light-emitting element 19 | M232 | 4.3 | 166% | 170% |
| Light-emitting element 20 | M246 | 4.4 | 132% | 131% |
| Light-emitting element 21 | M253 | 4.3 | 145% | 123% |
| Light-emitting element 22 | M265 | 4.5 | 170% | 141% |
| Light-emitting element 23 | M277 | 4.4 | 138% | 122% |
| Light-emitting element 24 | M286 | 4.4 | 148% | 144% |
| Light-emitting element 25 | M290 | 4.3 | 143% | 139% |
| Light-emitting element 26 | M295 | 4.4 | 130% | 146% |
| Light-emitting element 27 | N27 | 4.4 | 162% | 134% |
| Light-emitting element 28 | N36 | 4.5 | 157% | 147% |
| Light-emitting element 29 | N45 | 4.4 | 143% | 136% |
| Light-emitting element 30 | N75 | 4.4 | 160% | 155% |
| Light-emitting element 31 | N95 | 4.4 | 128% | 142% |
| Light-emitting element 32 | N126 | 4.4 | 131% | 128% |
| Light-emitting element 33 | N173 | 4.3 | 137% | 135% |
| Light-emitting element 34 | N236 | 4.4 | 143% | 141% |
| Light-emitting element 35 | N262 | 4.5 | 149% | 132% |
| Light-emitting element 36 | N294 | 4.4 | 128% | 144% |
| Light-emitting element 37 | N318 | 4.3 | 127% | 139% |
| Light-emitting element 38 | N341 | 4.4 | 169% | 134% |
| Light-emitting element 39 | N345 | 4.4 | 156% | 131% |
| Comparative element 1 | Ref 1 | 4.8 | 100% | 100% |
| Comparative element 2 | Ref 2 | 4.5 | 94% | 95% |
| Comparative element 3 | Ref 3 | 5.8 | 30% | 41% |
| Comparative element 4 | Ref 4 | 4.5 | 75% | 77% |

As can be seen from Table 4, in the light-emitting elements 1 to 39 obtained by using the organic compounds obtained in examples 1 to 39 as hole transport materials in the hole transport layer, two fluorenyl groups of the organic compounds are connected by a single bond and are respectively connected to one aromatic amine group, the two fluorenyl groups form an approximately orthogonal highly twisted conformation, the energy level distribution is more uniform, the hole transport capacity is stronger and the stability is higher, and the energy level barrier between the hole injection layer and the light-emitting auxiliary layer can be effectively reduced, which is beneficial to carrier transport, and both the luminous efficiency and the lifetime are significantly improved compared with the comparative elements 1 to 4.

In a case that the organic compounds obtained in examples 1 to 39 are used as hole transport materials, the organic compounds obtained in examples 1 to 39 have a bis-arylamine structure compared with Ref 1 and Ref 2 as hole transport materials, have a larger molecular weight, a higher molecular twist, and a more uniform energy level distribution. In a case that the organic compounds obtained in examples 1 to 39 are used as hole transport materials, the energy levels of the organic compounds obtained in examples 1 to 39 are more matched and have better hole transport properties that of Ref 3 as hole transport materials because of the presence of nitrogen atoms. In a case that the organic compounds obtained in examples 1 to 39 are used as the hole transport materials, two aromatic amine groups are each attached to one fluorenyl group, and the energy level distribution is more uniform than that of Ref 4 as the hole-transporting material.

According to the light-emitting element disclosed in the embodiments of the present disclosure, the organic compounds are used. In the structure of the organic compound, two fluorenyl groups are connected by a single bond to form an approximately orthogonal highly twisted conformation, and two fluorenyl groups are respectively connected to one aromatic amine group, so that the molecular structure is further twisted, the energy level distribution of the molecule is more uniform, and the hole transport capacity of the organic compound is stronger and the stability is higher, and the transport balance of the holes and the electrons in the light-emitting element are adjusted to be stronger, thereby improving the luminous efficiency of the light-emitting element and prolonging the service life of the light-emitting element.

Embodiments of the present disclosure also discloses a display panel including a light-emitting element as described above.

The display panel further includes an array substrate disposed on one side of the light-emitting element, and an encapsulation layer disposed on one side of the light-emitting element away from the array substrate and covering the light-emitting element. The display panel further includes a polarizer layer disposed on one side of the encapsulation layer away from the light-emitting element and a cover plate layer disposed on one side of the polarizer layer away from the light-emitting element. The polarizer layer may be replaced by a color filter layer, and the color filter layer may include a plurality of color resistors and a black matrix disposed on both sides of the color resistors.

According to the display panel disclosed in the embodiments of the present disclosure, organic compounds having structures shown in formula (1) are used in the light-emitting element. In the structure of the organic compounds, two fluorenyl groups are connected by a single bond to form an approximately orthogonal highly twisted conformation, and two fluorenyl groups are respectively connected to one aromatic amine group, so that the molecular structure is further twisted, the energy level distribution of the molecule is more uniform, and the hole transport capacity of the organic compound is stronger and the stability is higher, and the transport balance of the holes and the electrons in the light-emitting element are adjusted to be stronger, thereby improving the luminous efficiency of the light-emitting element and prolonging the service life of the light-emitting element.

Embodiments of the present disclosure discloses an organic compound, a light-emitting element and a display panel. The organic compound has a structure as shown in formula (1):

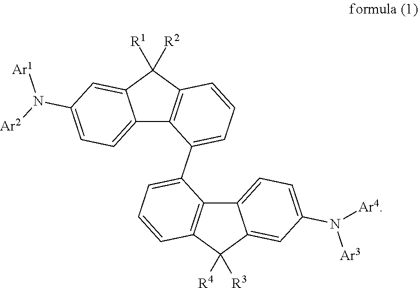

Organic compounds having structures shown in formula (1) are used in the present disclosure. In the structures of the organic compounds, two fluorenyl groups are connected by a single bond to form an approximately orthogonal highly twisted conformation, and two fluorenyl groups are respectively connected to one aromatic amine group, so that the molecular structure is further twisted, the energy level distribution of the molecule is more uniform, and the hole transport capacity of the organic compound is stronger and the stability is higher, and the transport balance of the holes and the electrons in the light-emitting element are adjusted to be stronger, thereby improving the luminous efficiency of the light-emitting element and prolonging the service life of the light-emitting element.

It can be understood by those skilled in the art can make equivalent substitutions or changes according to the technical solutions of the present disclosure and its inventive concept, and all these changes or substitutions should fall within the protection scope of the appended claims of the present disclosure.

What is claimed is:

1. An organic compound, wherein the organic compound has a structure as shown in formula (1): 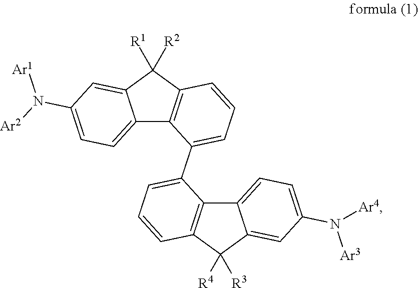
wherein Ar¹, Ar², Ar³ and Ar⁴ are each independently selected from a substituted or unsubstituted aromatic group having 6 to 20 ring atoms and a substituted or unsubstituted heteroaromatic group having 5 to 30 ring atoms; and
R¹, R², R³ and R⁴ are each independently selected from a substituted or unsubstituted methyl, a substituted or unsubstituted ethyl, a substituted or unsubstituted phenyl.

2. The organic compound according to claim 1, wherein Ar¹, Ar², Ar³, Ar⁴ are each independently selected from groups as follows: 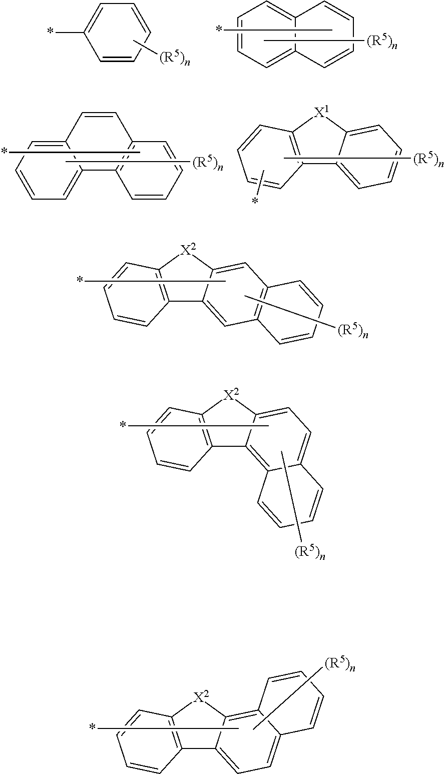
wherein X¹ and X² are each independently selected from O, S, N-Ph, CR⁶R⁷;
R⁵, R⁶ and R⁷ are each independently selected from hydrogen, deuterium, a substituted or unsubstituted linear alkyl group having 1 to 20 carbon atoms, a substituted or unsubstituted branched alkyl group or cyclic alkyl group having 3 to 20 carbon atoms, a substituted or unsubstituted aromatic group having 6 to 10 ring atoms;
N is selected from any integer from 0 to 9; and
* indicates a linkage site.

3. The organic compound according to claim 2, wherein X¹ is selected from O, S, N-Ph, CR⁶R⁷, and X² is selected from O, S, CR⁶R⁷;
each occurrence of R⁵ is independently selected from hydrogen, deuterium, a substituted or unsubstituted methyl, a substituted or unsubstituted branched alkyl or cyclic alkyl having 3 to 10 carbon atoms, a substituted or unsubstituted phenyl, a substituted or unsubstituted naphthyl; and
R⁶ and R⁷ are each independently selected from a substituted or unsubstituted methyl, a substituted or unsubstituted ethyl, a substituted or unsubstituted phenyl.

4. The organic compound according to claim 3, wherein each occurrence of R⁵ is independently selected from hydrogen, deuterium, an unsubstituted methyl, an unsubstituted isopropyl, an unsubstituted tert-butyl, an unsubstituted cyclohexyl, an unsubstituted adamantyl, an unsubstituted phenyl, an unsubstituted naphthyl; and
R⁶ and R⁷ are each independently selected from an unsubstituted methyl, an unsubstituted ethyl, an unsubstituted phenyl.

5. The organic compound according to claim 3, wherein Ar¹, Ar², Ar³ and Ar⁴ are each independently selected from groups as follows:

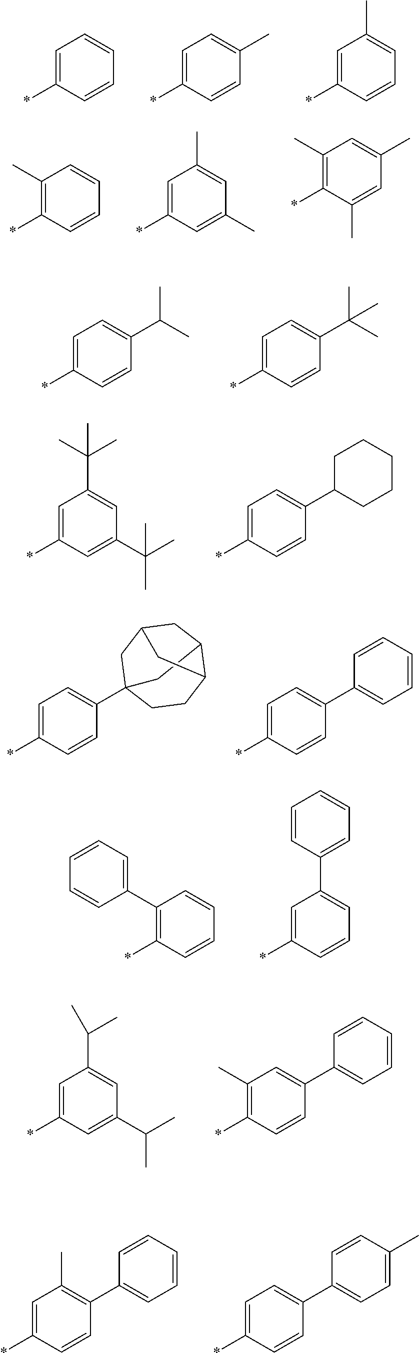

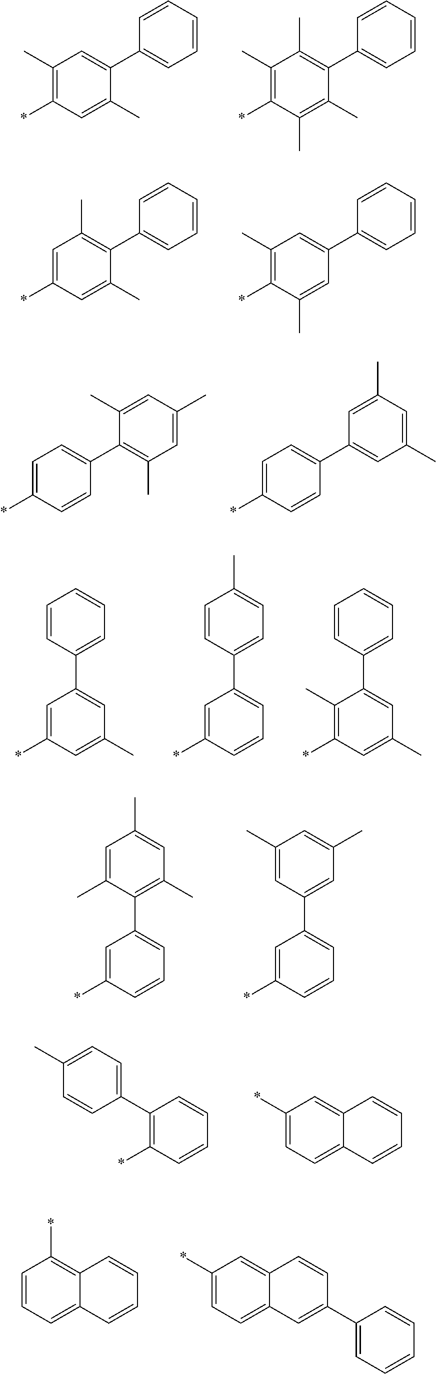

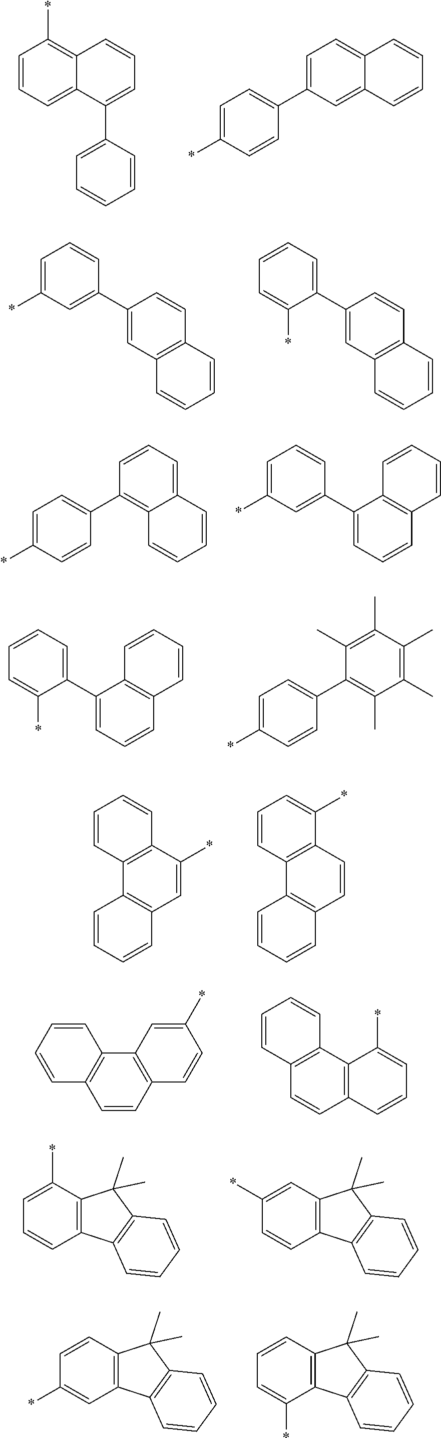

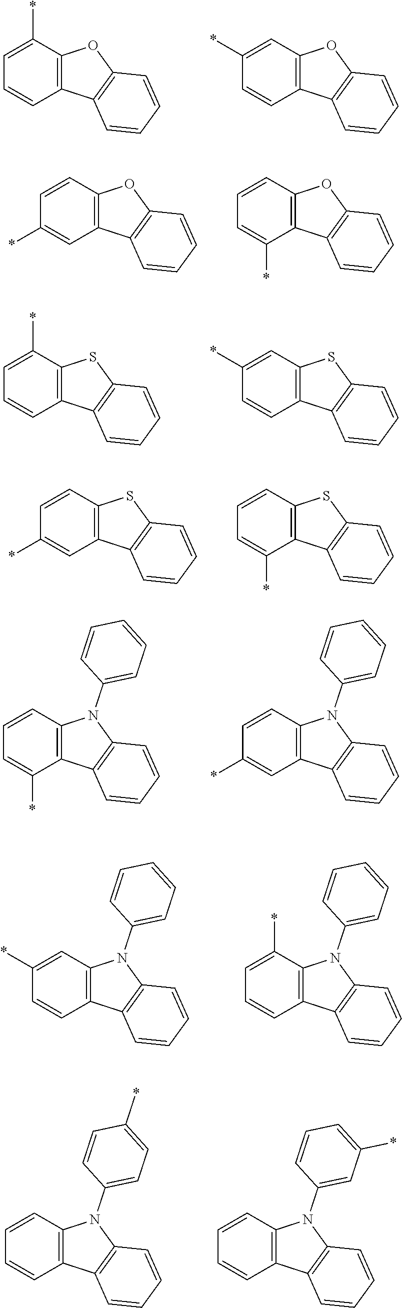

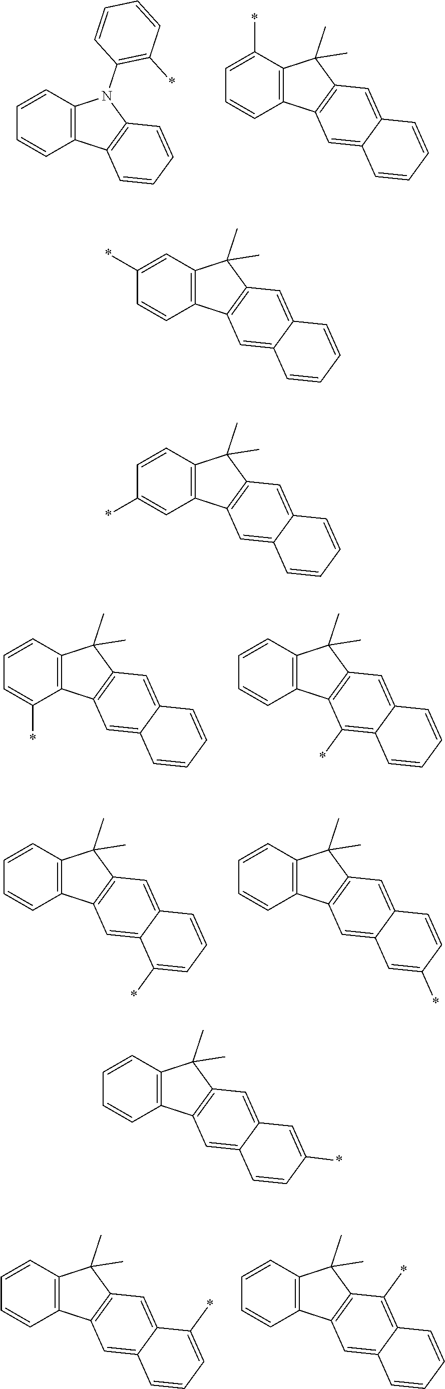

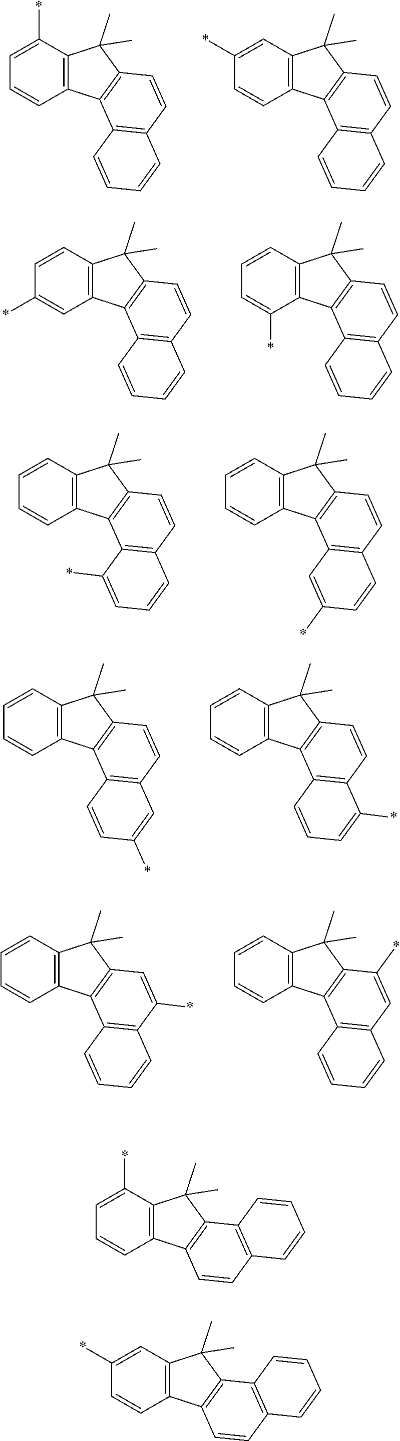

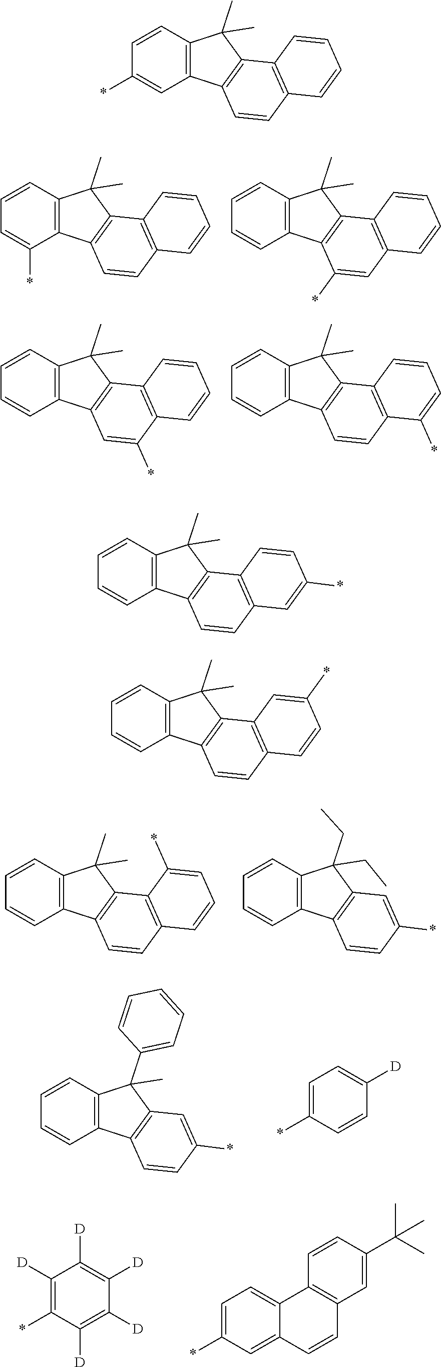

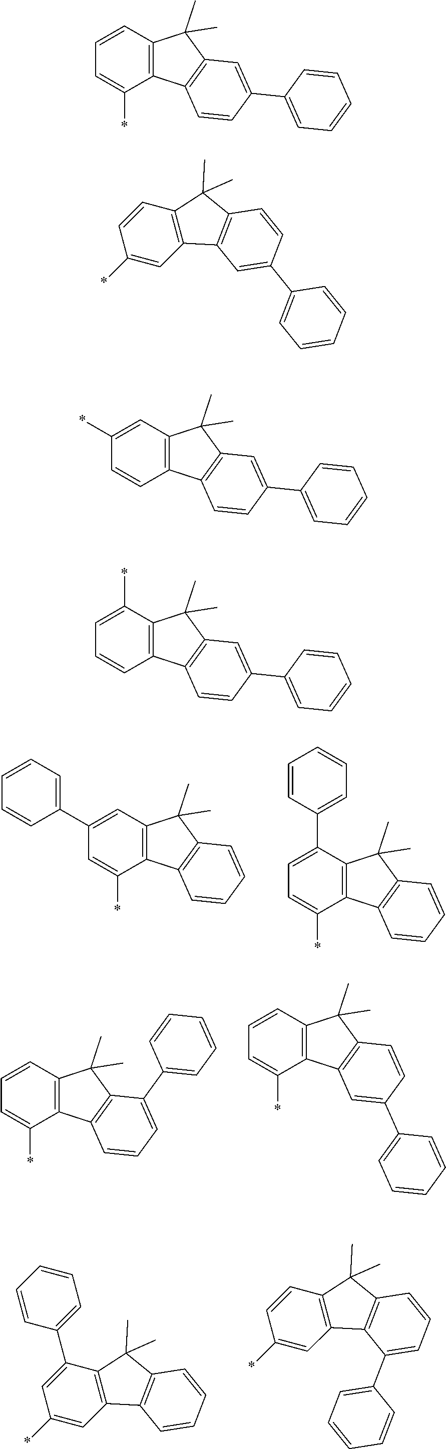

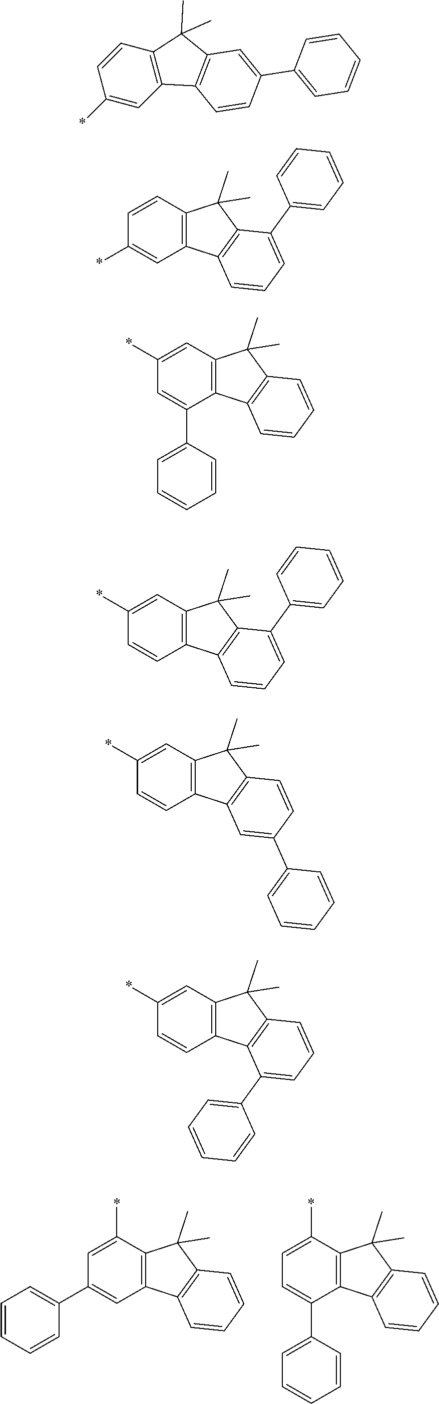

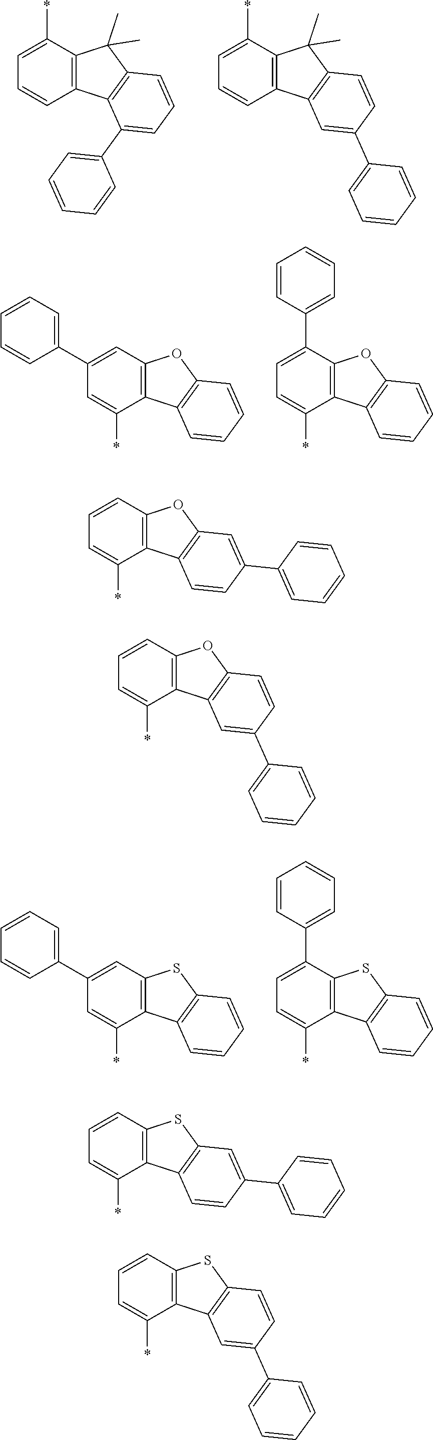

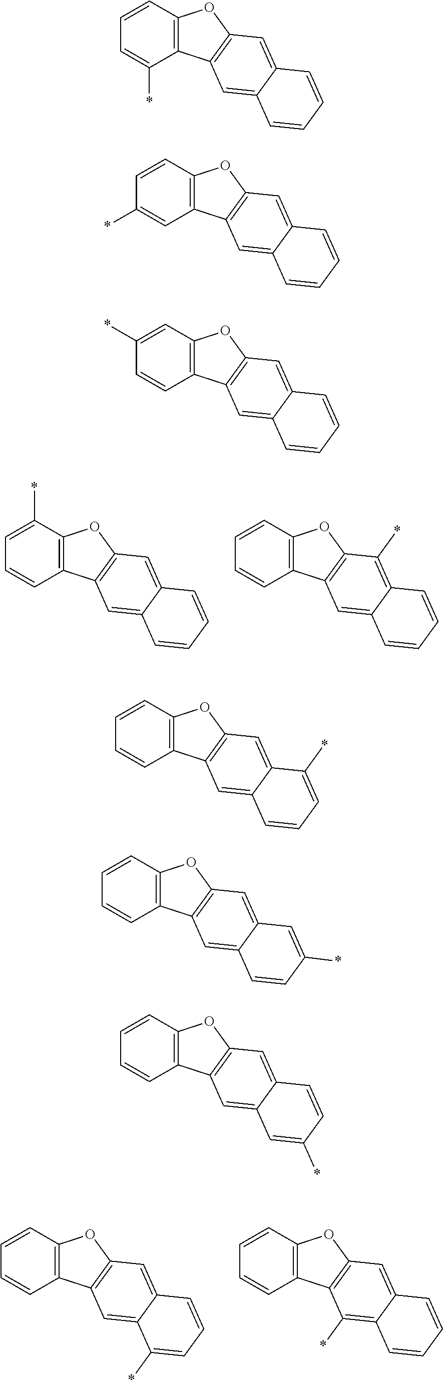

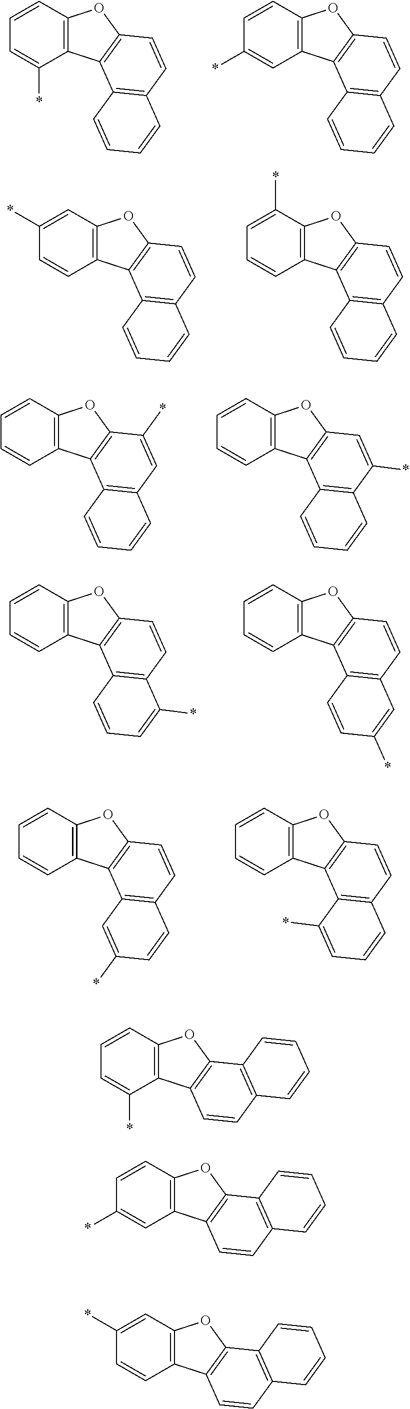

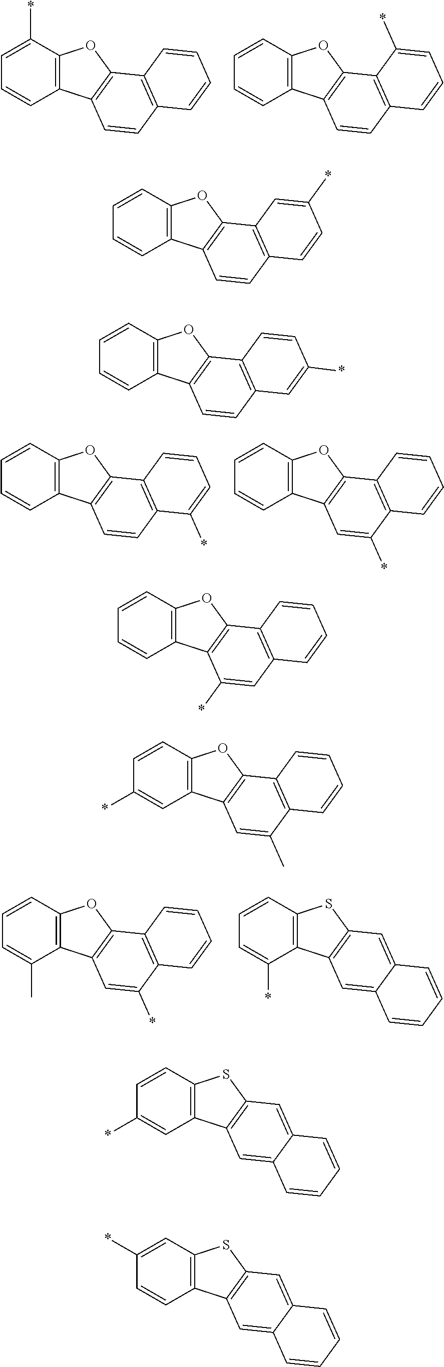

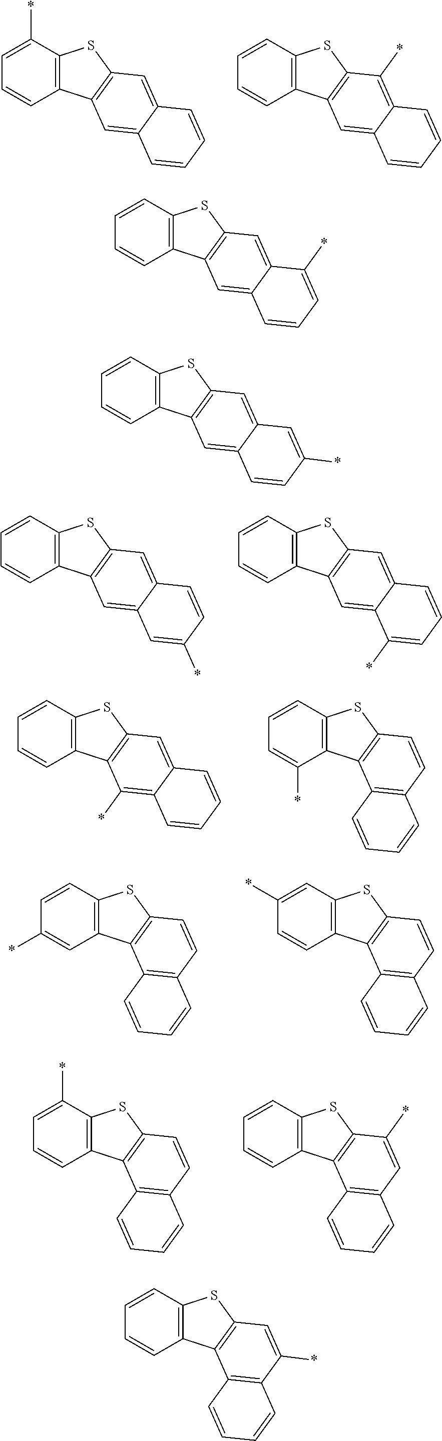

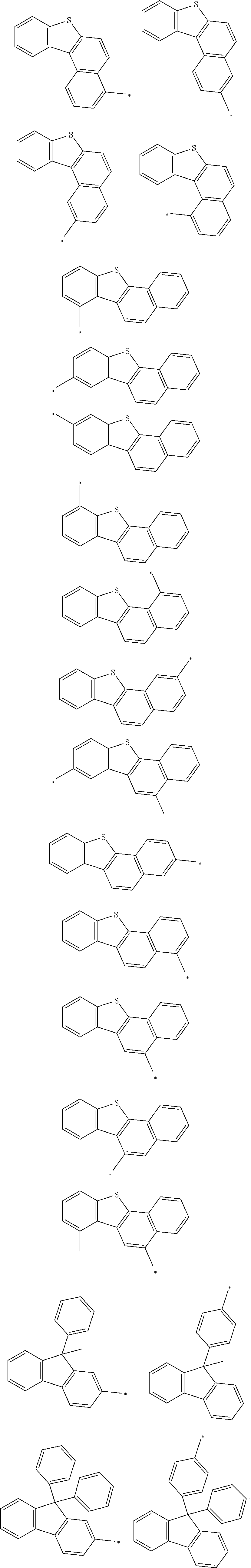

6. The organic compound according to claim 1, wherein Ar¹ is centrosymmetric with Ar³.

7. The organic compound according to claim 1, wherein Ar² is centrosymmetric with Ar⁴.

8. The organic compound according to claim 1, wherein the substituted methyl, the substituted ethyl, and the substituted phenyl satisfy the following conditions:
at least one hydrogen atom in the groups is replaced by a deuterium atom.

9. The organic compound according to claim 1, wherein R¹, R², R³, R⁴ are each independently selected from an unsubstituted methyl, an unsubstituted ethyl, an unsubstituted phenyl,

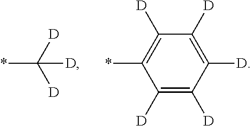

10. The organic compound according to claim 1, wherein the organic compound is selected from compounds as follows:

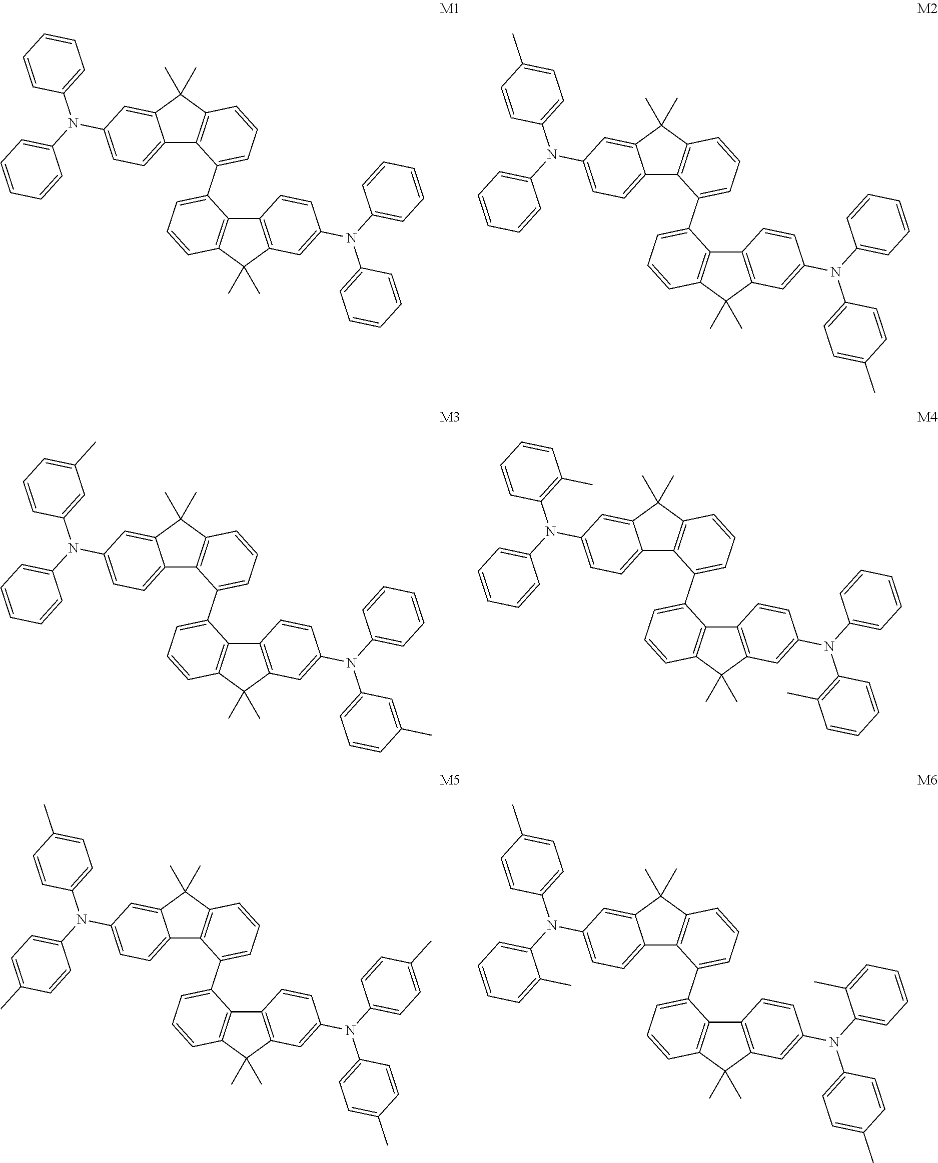

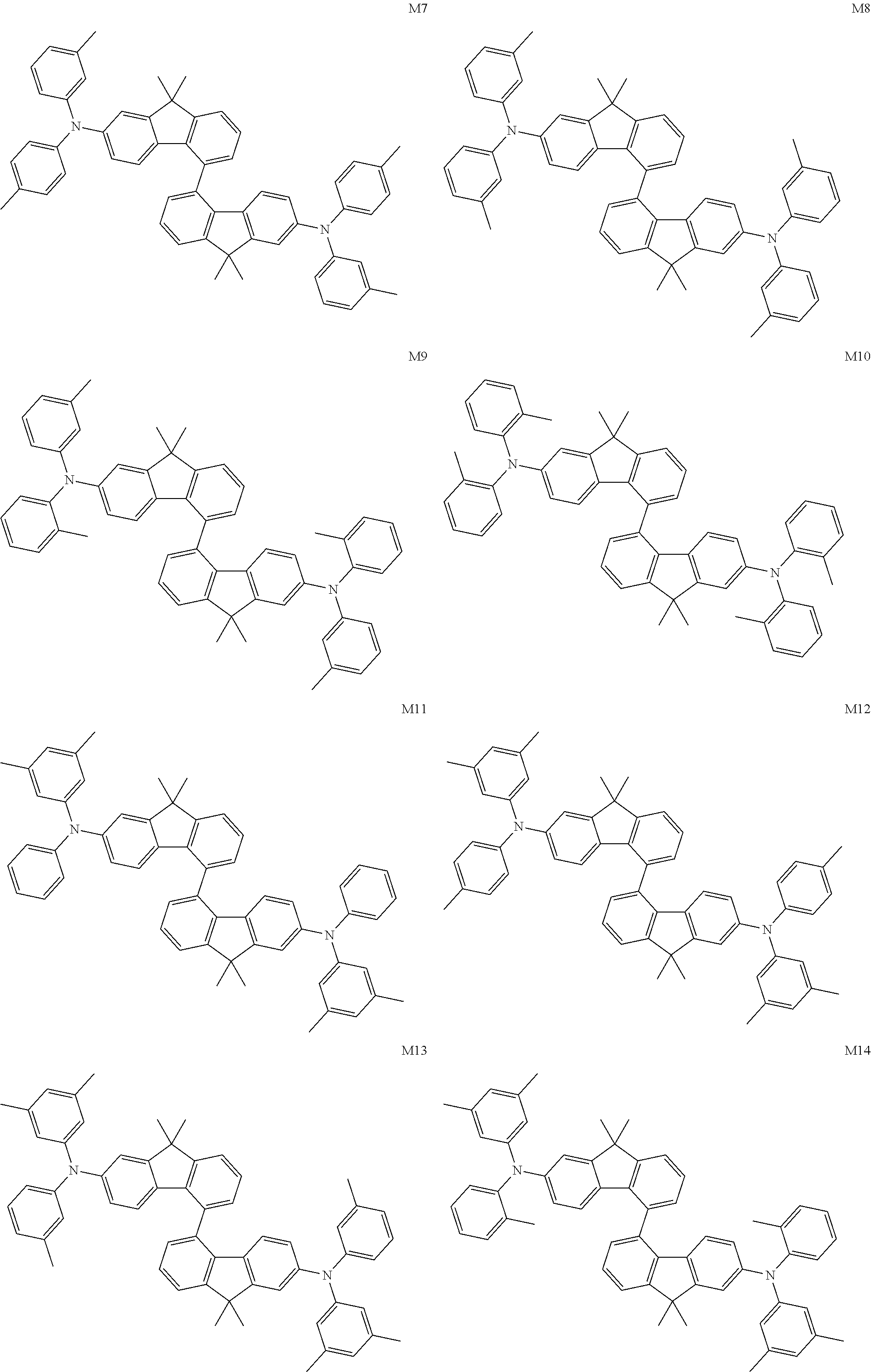

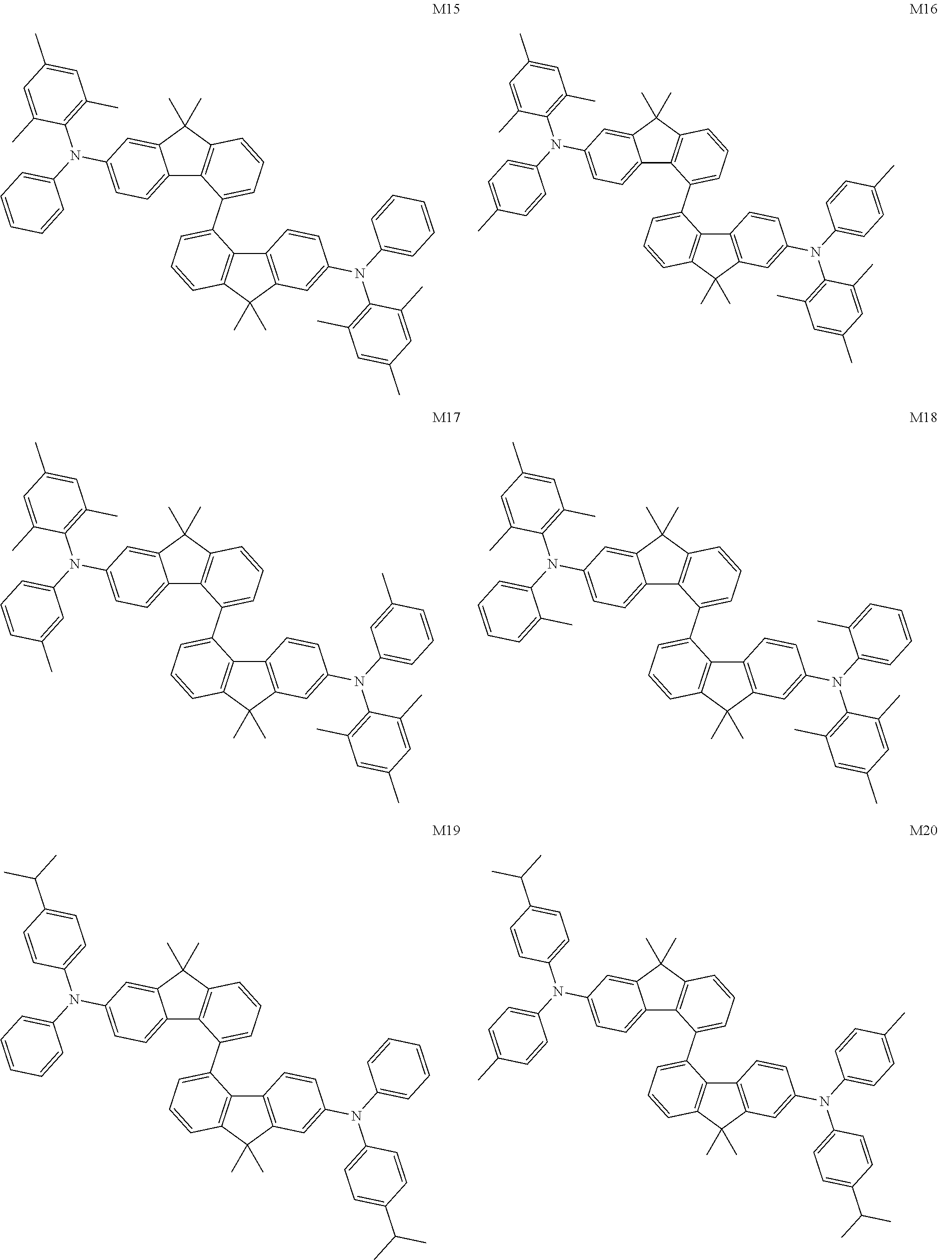

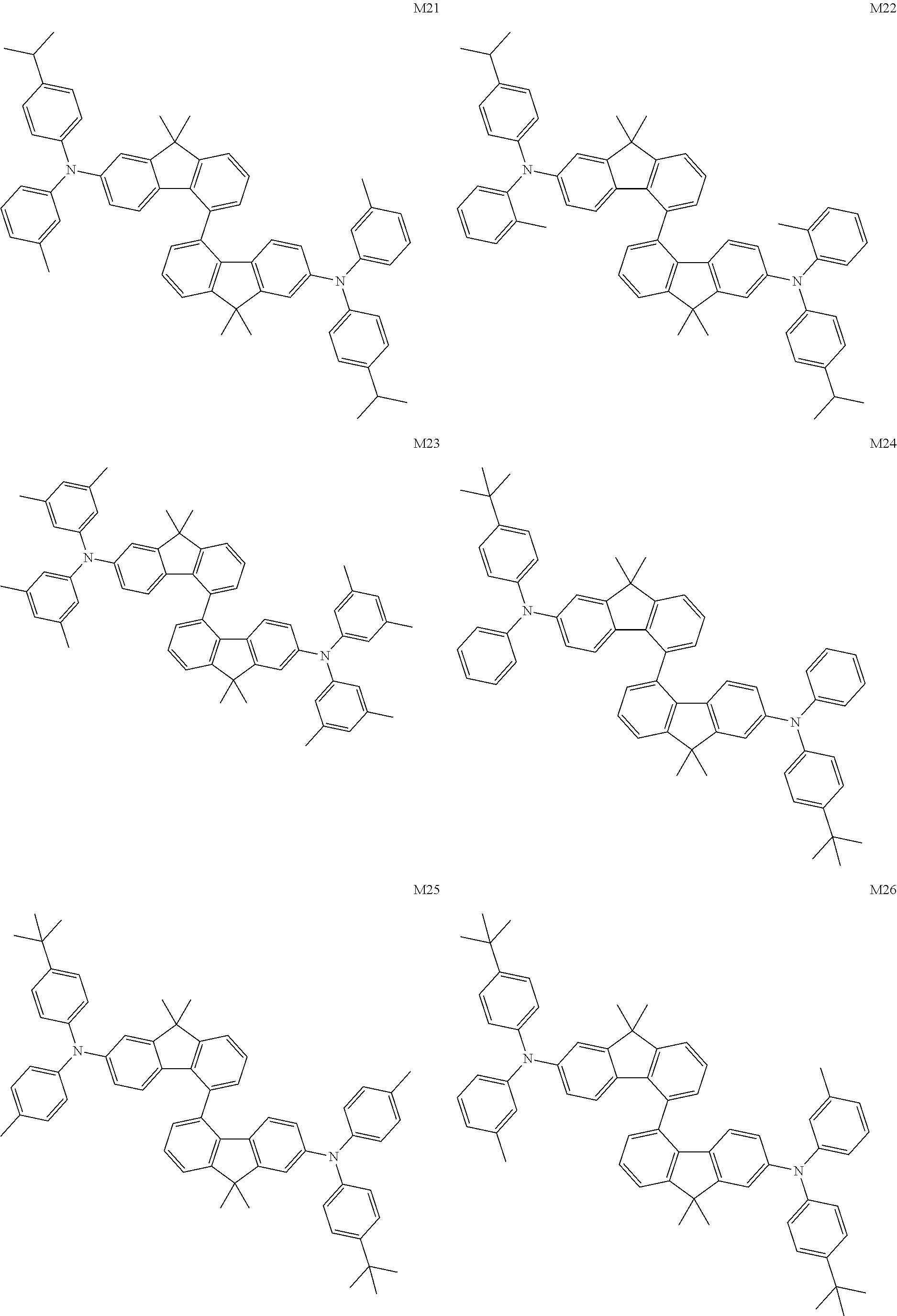

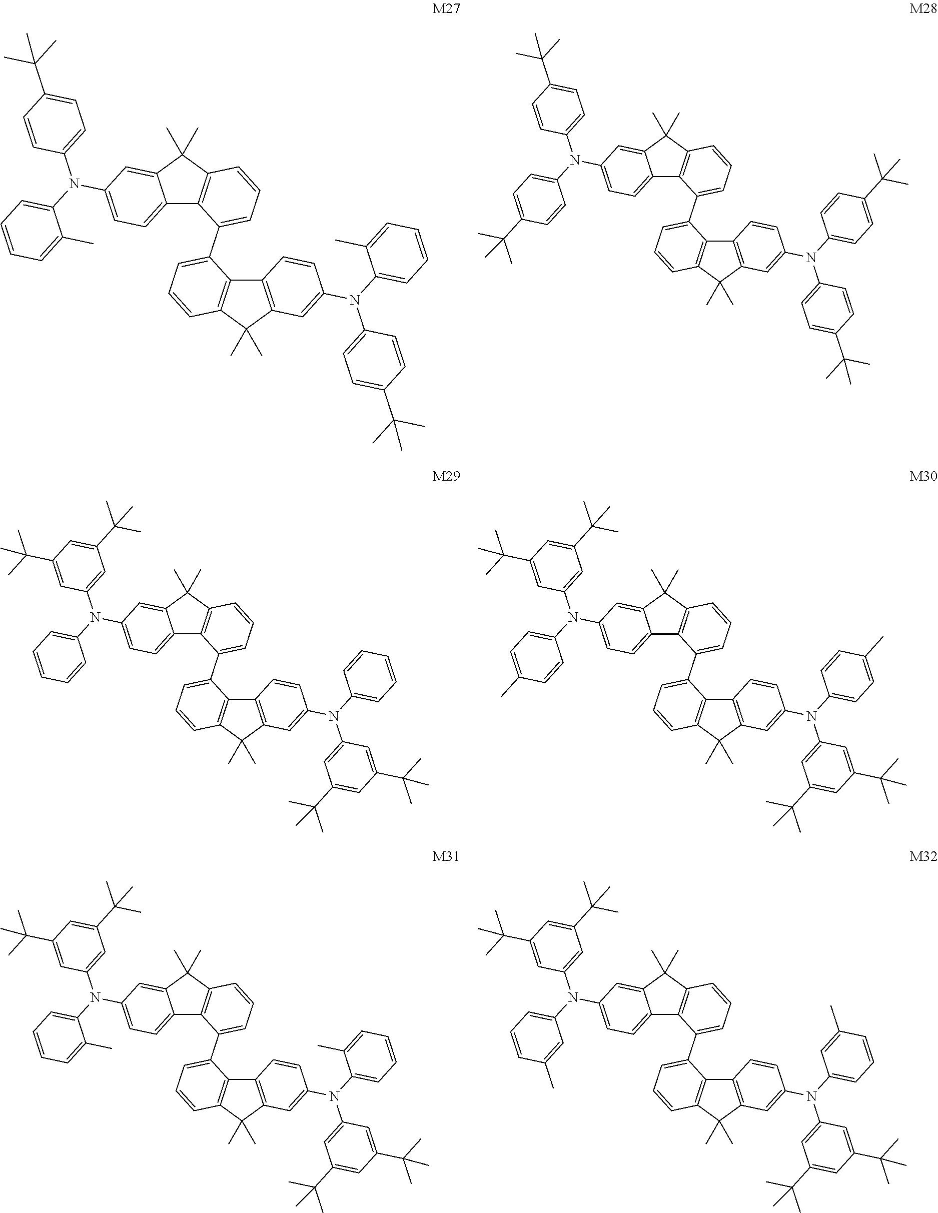

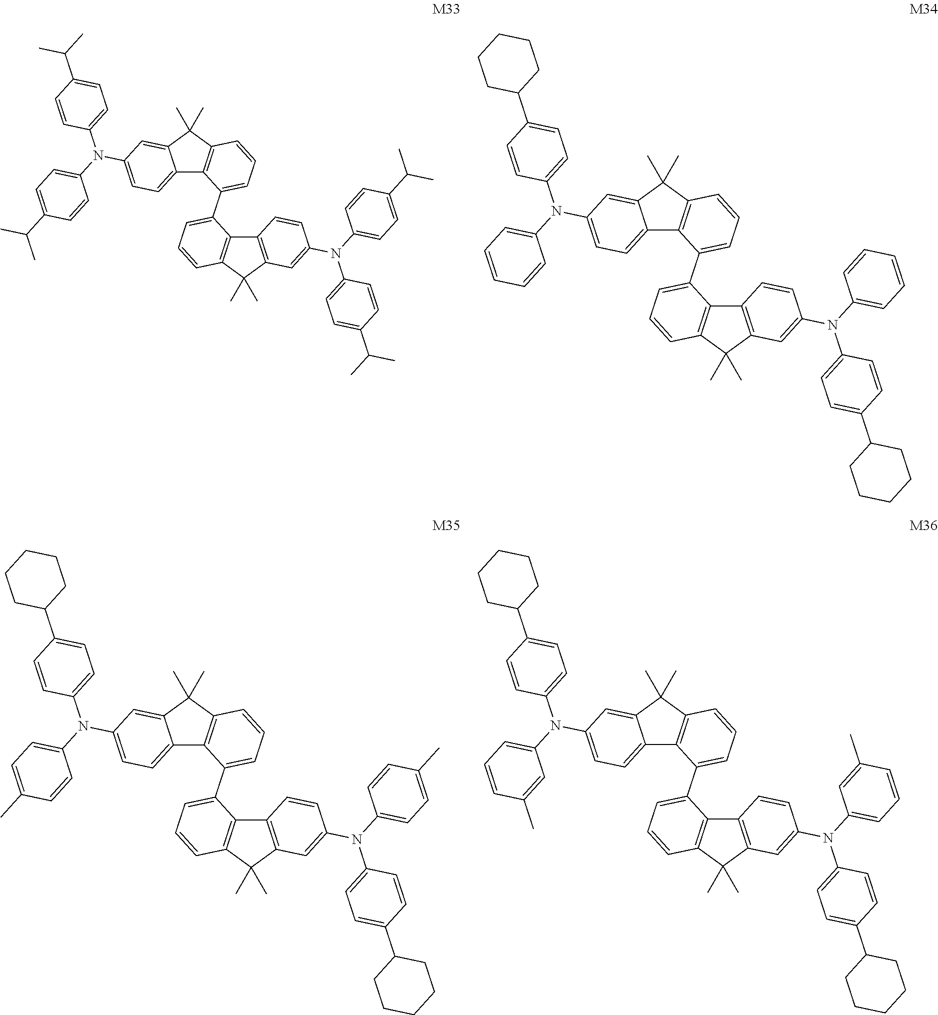

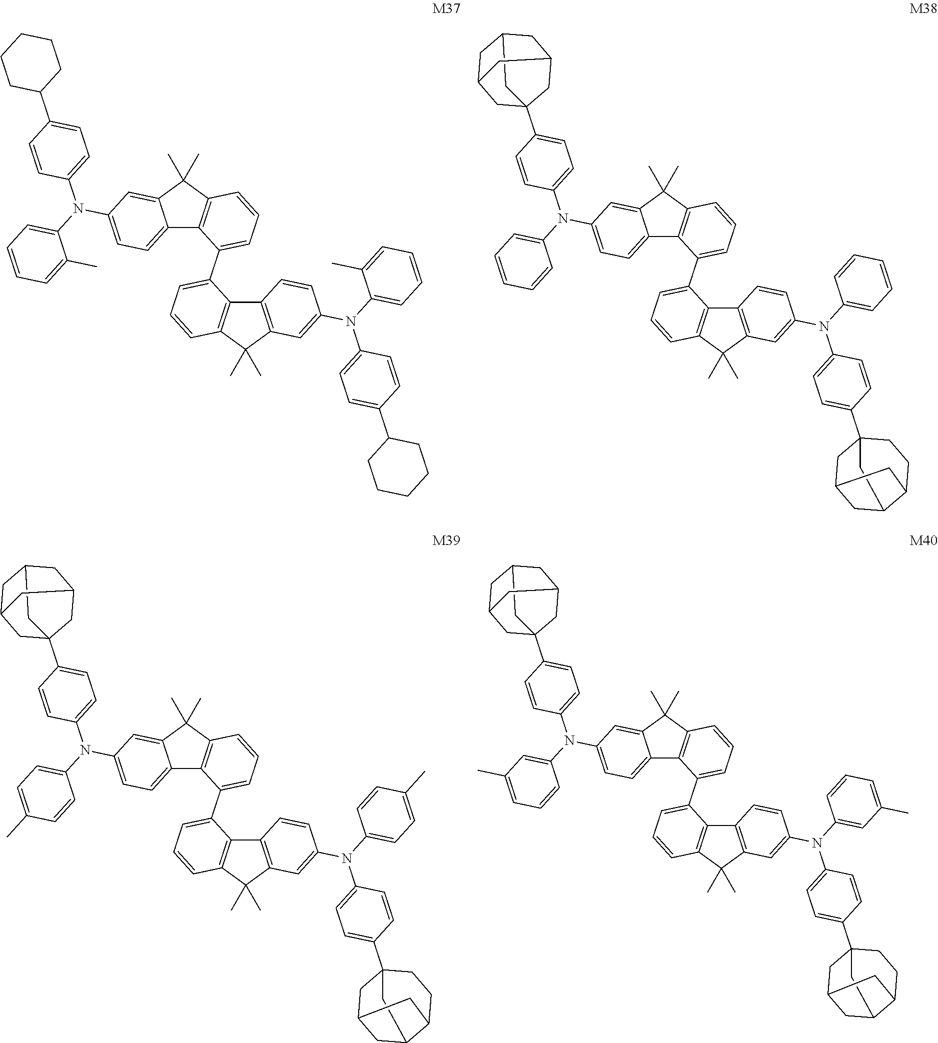

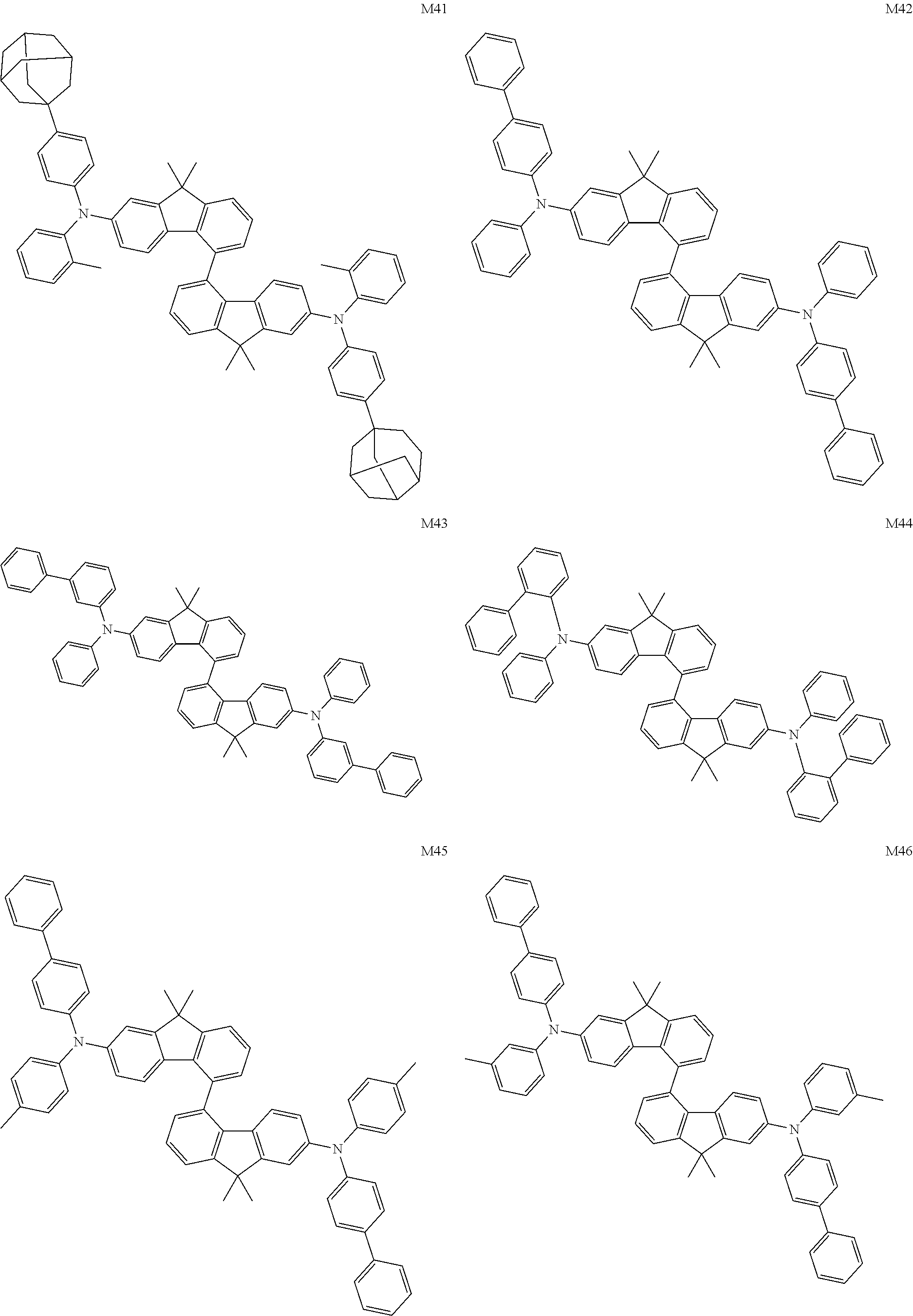

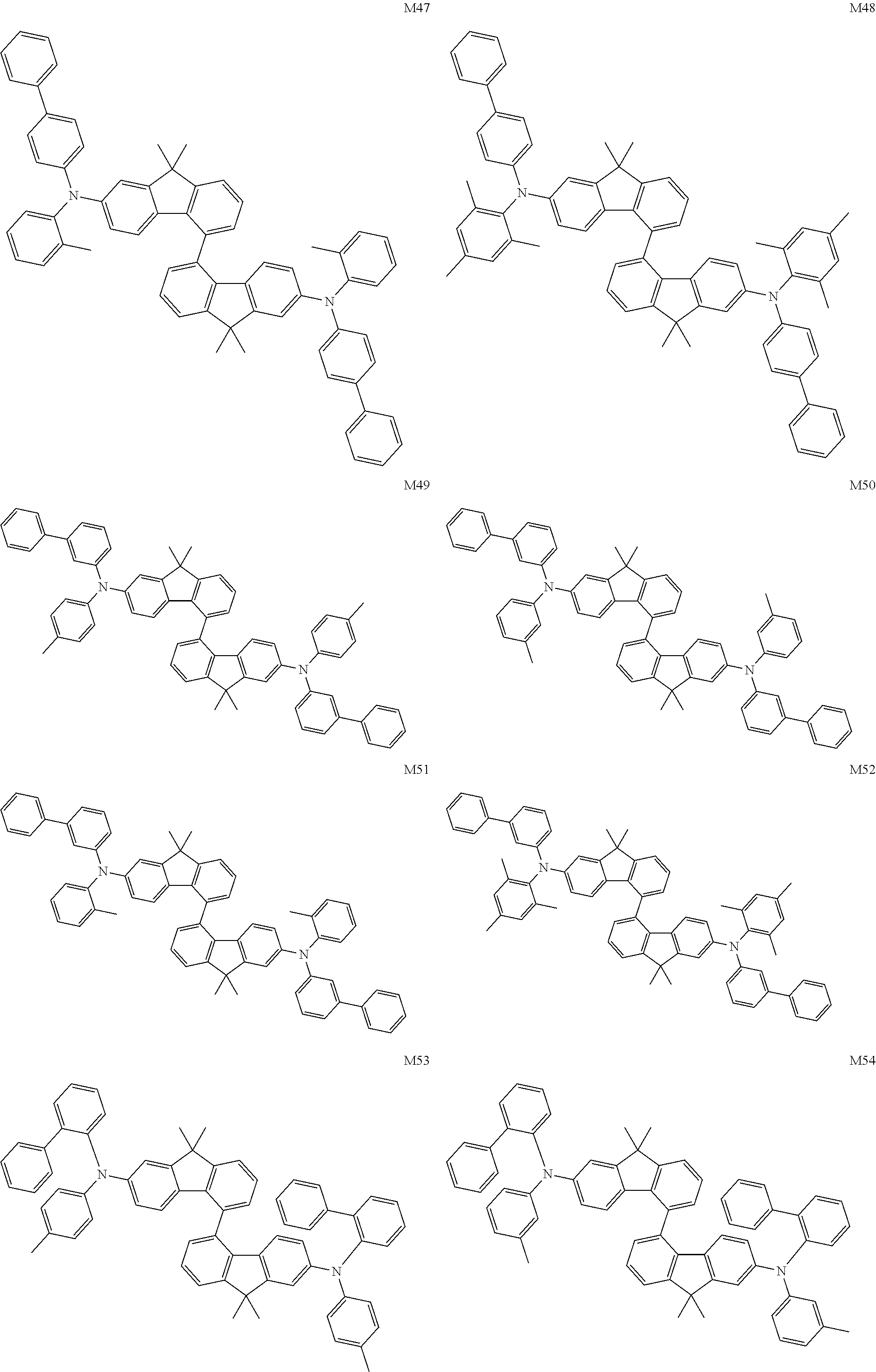

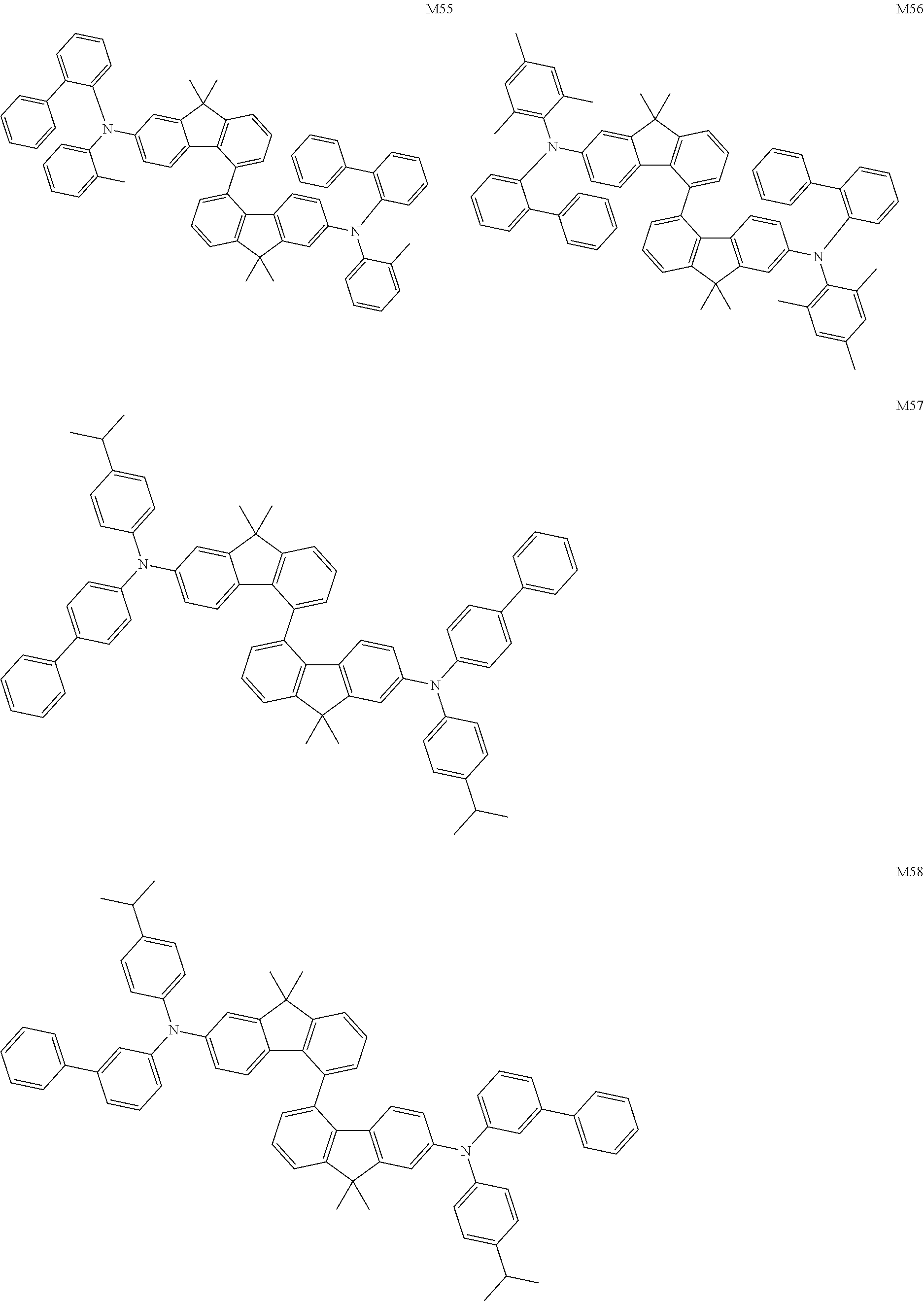

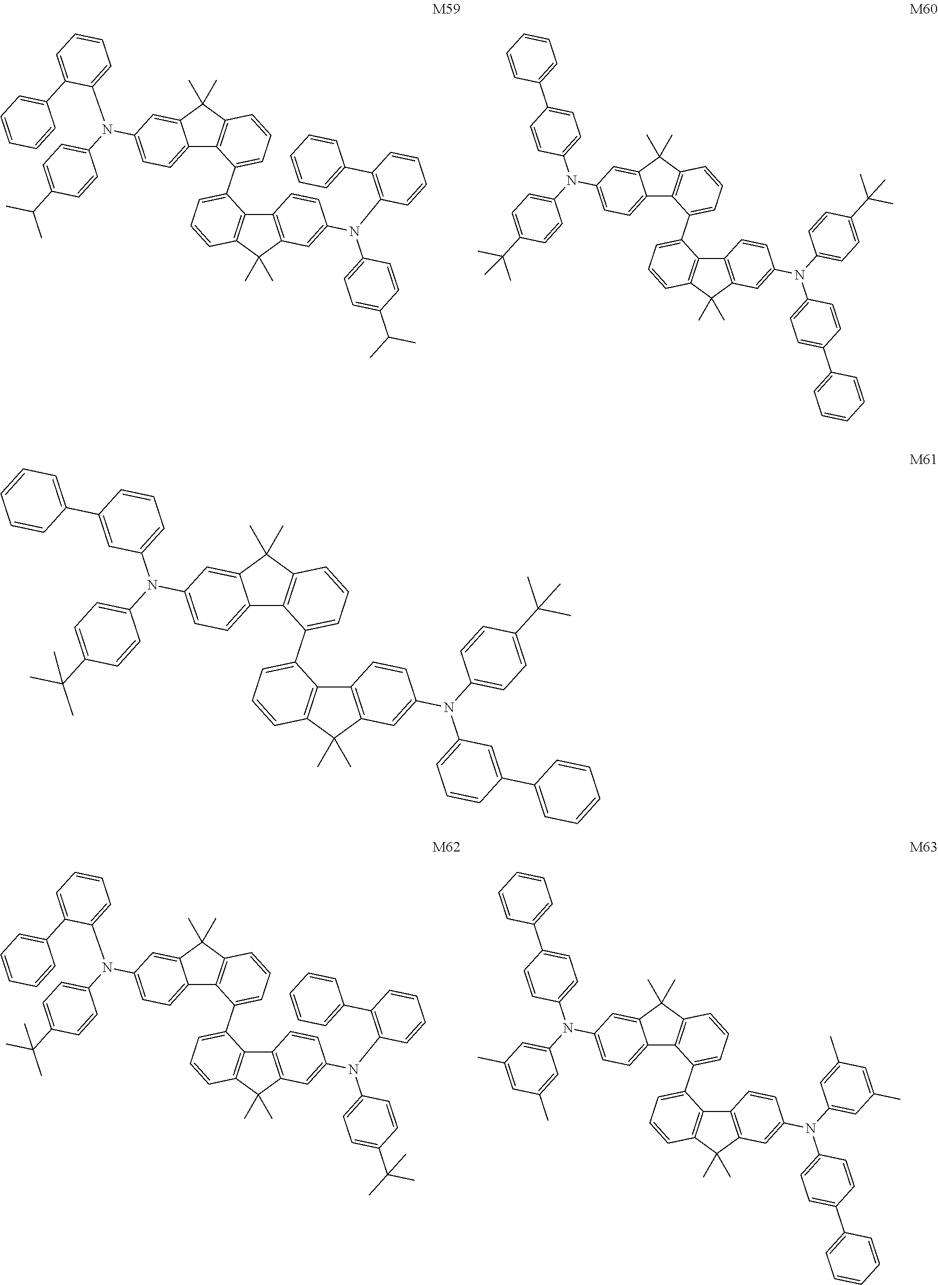

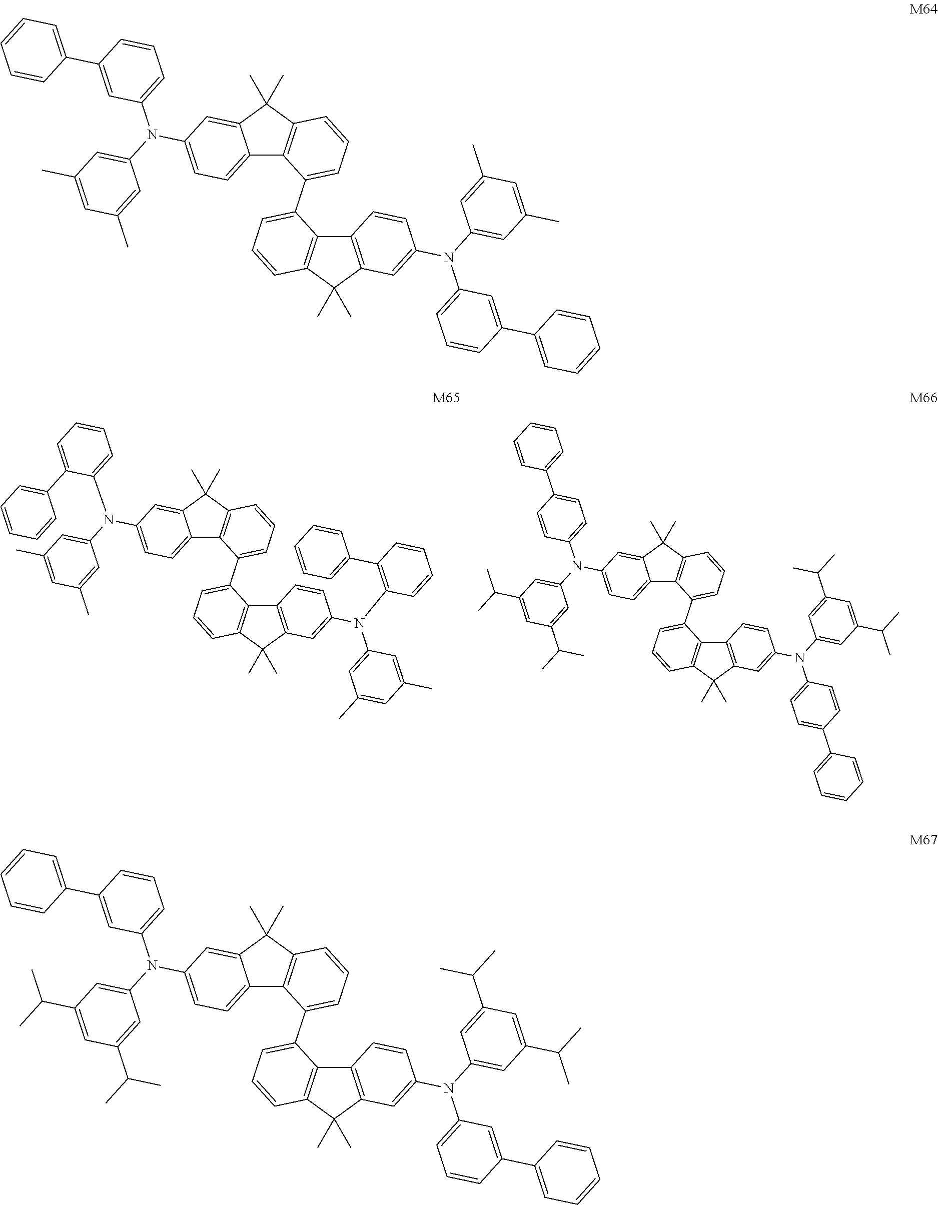

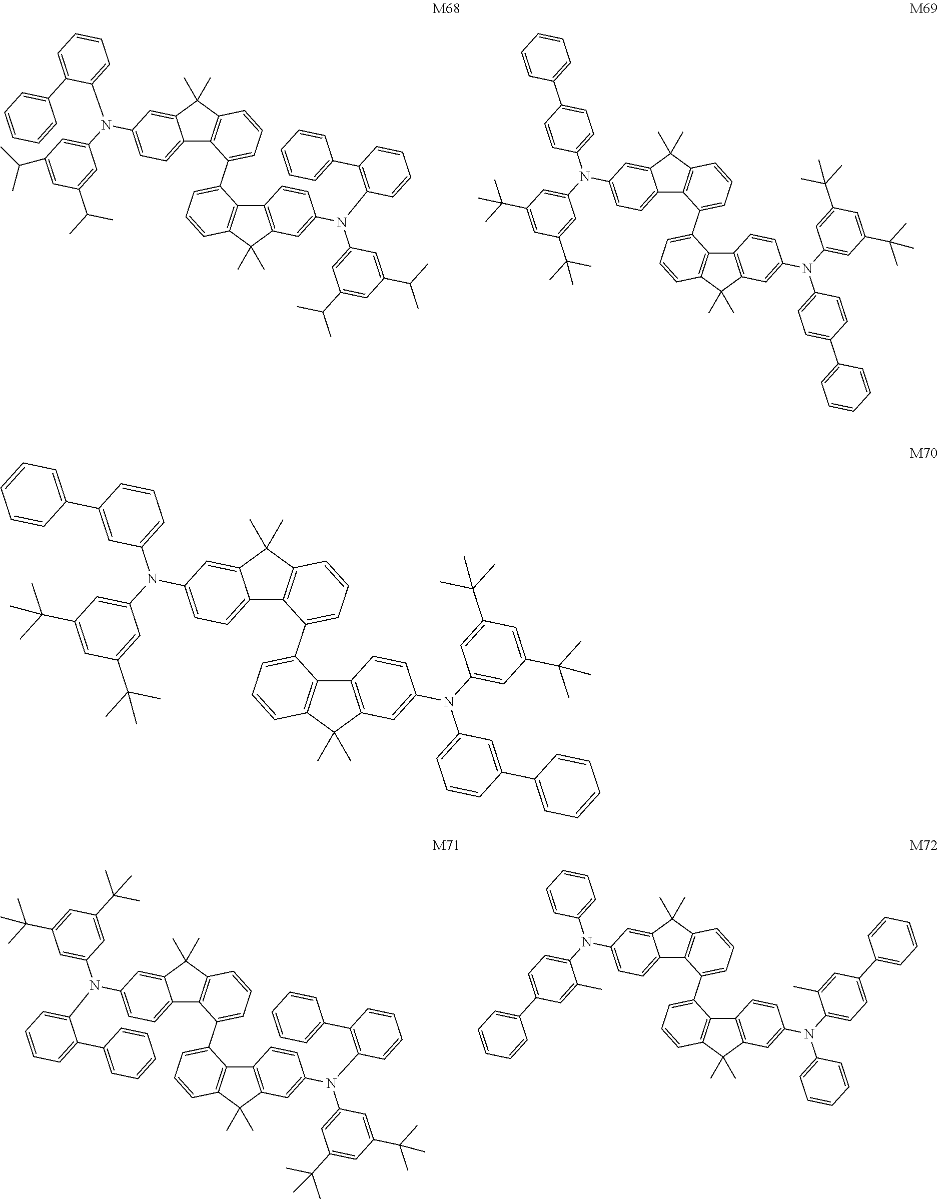

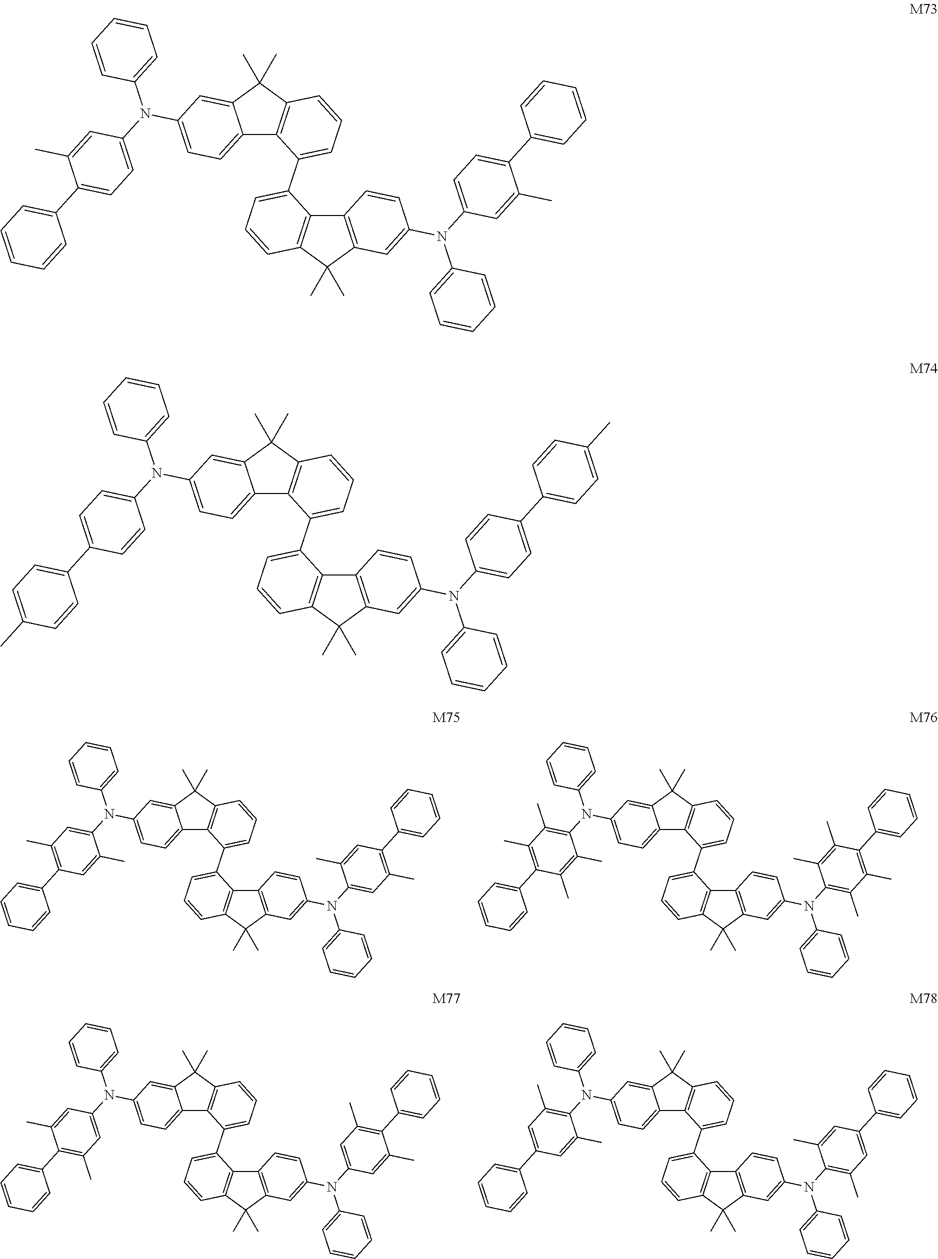

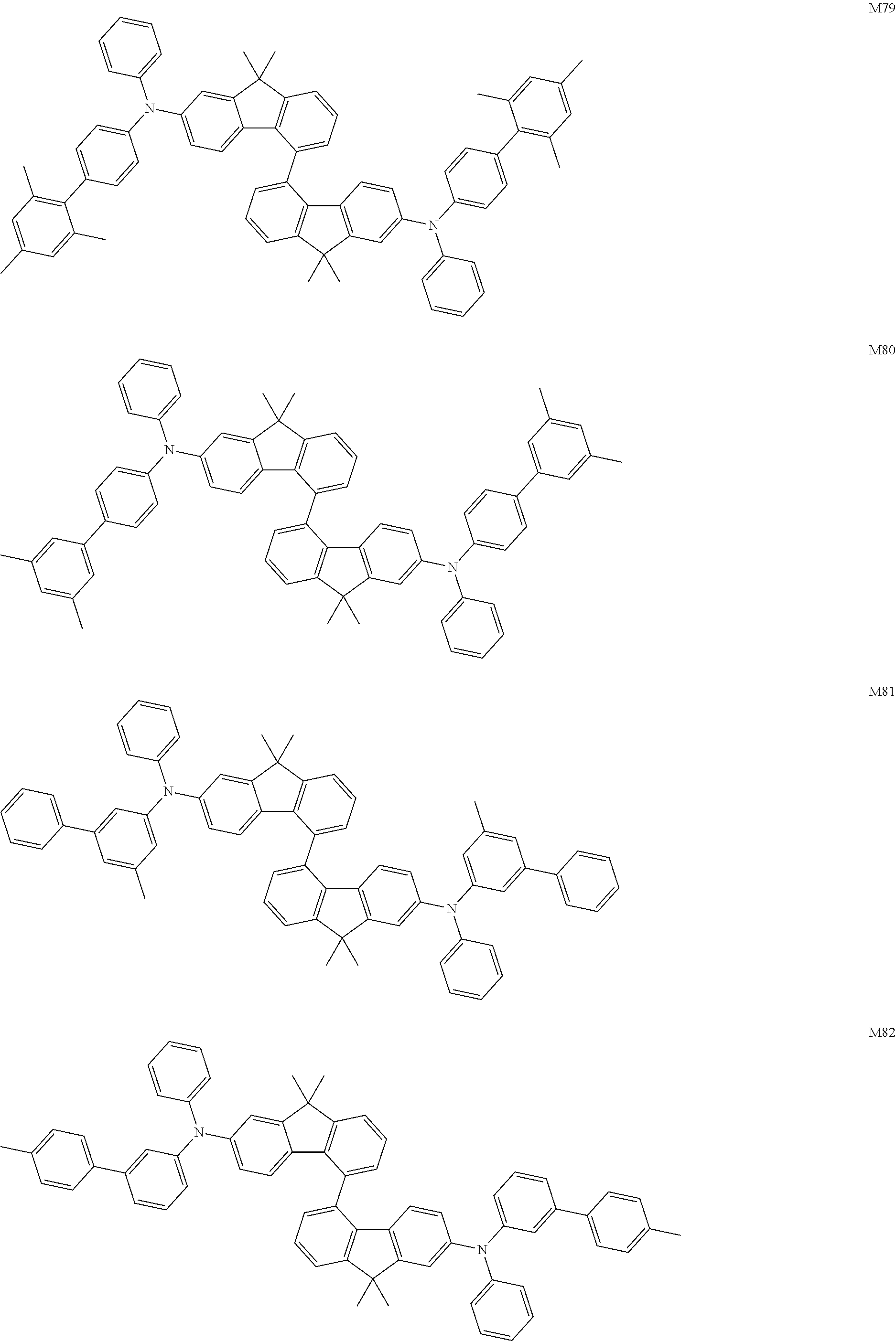

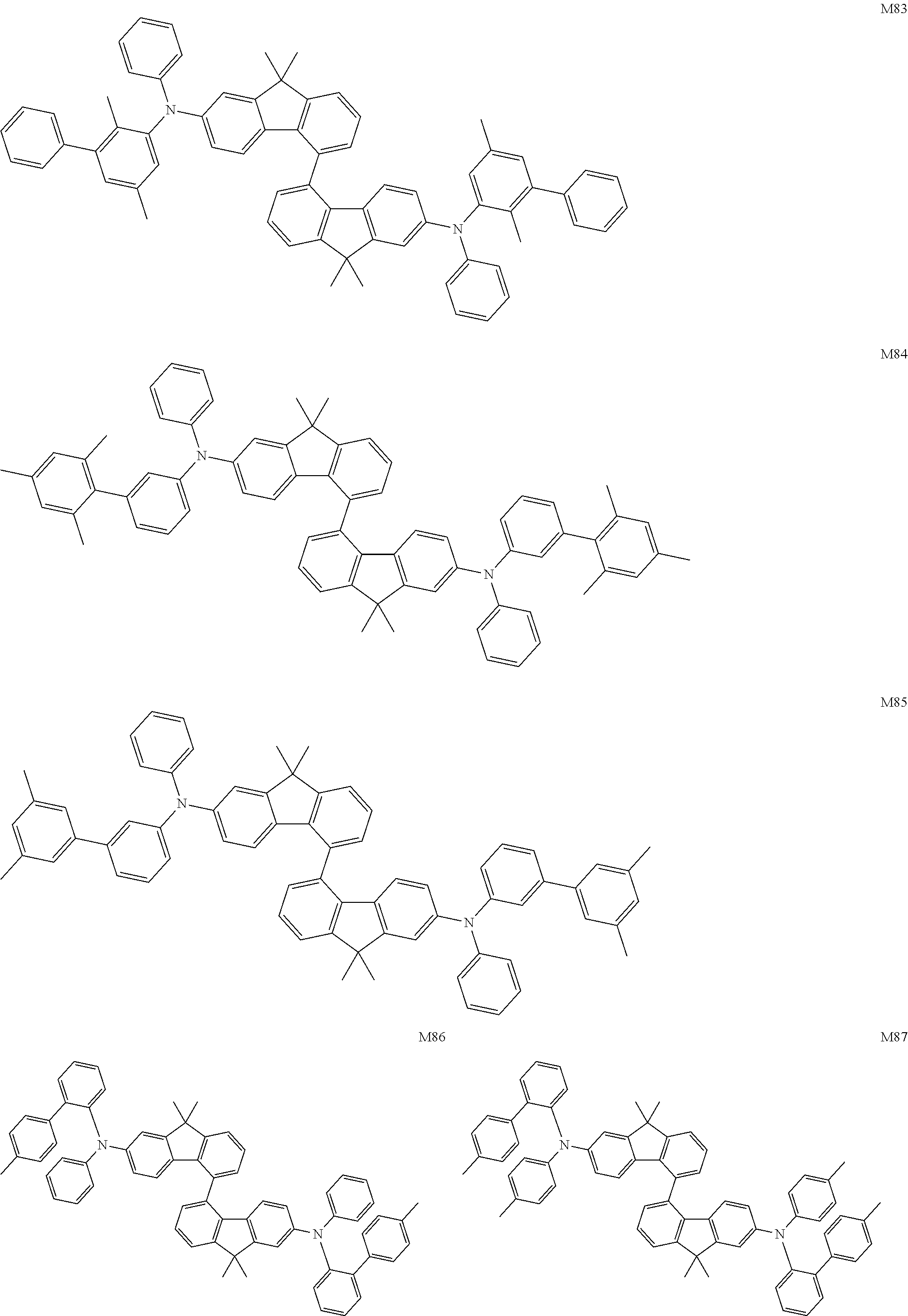

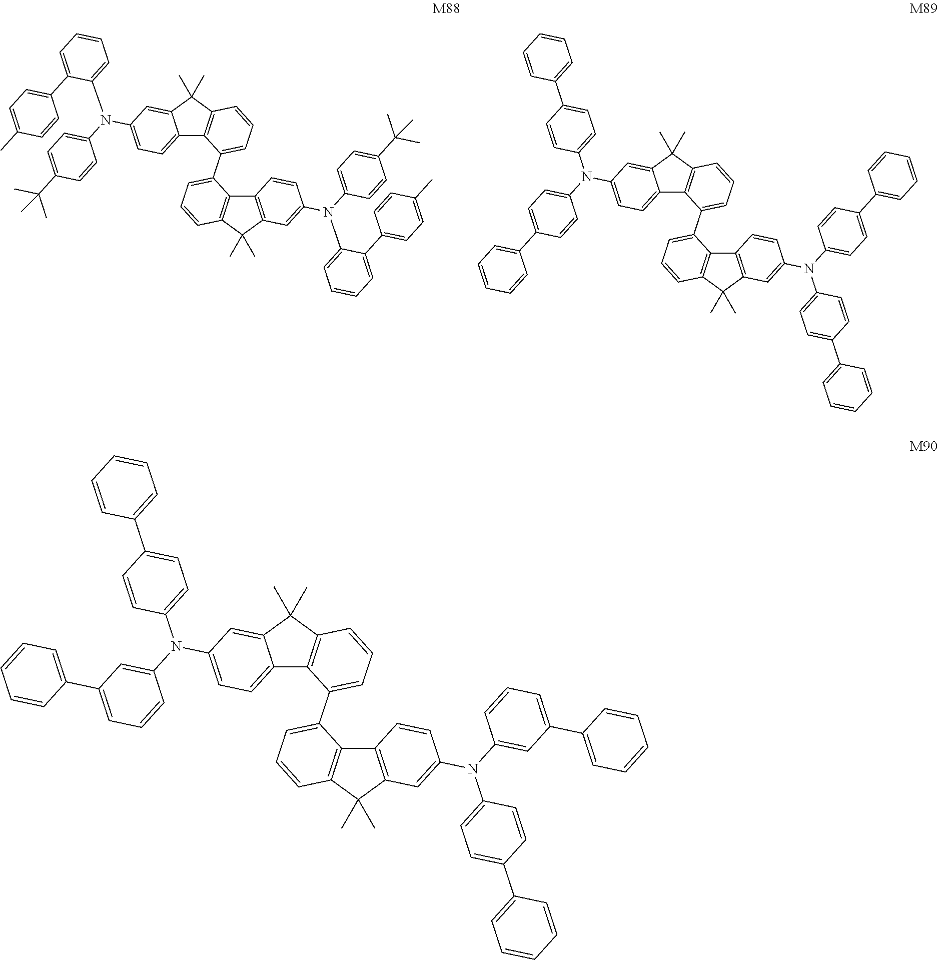

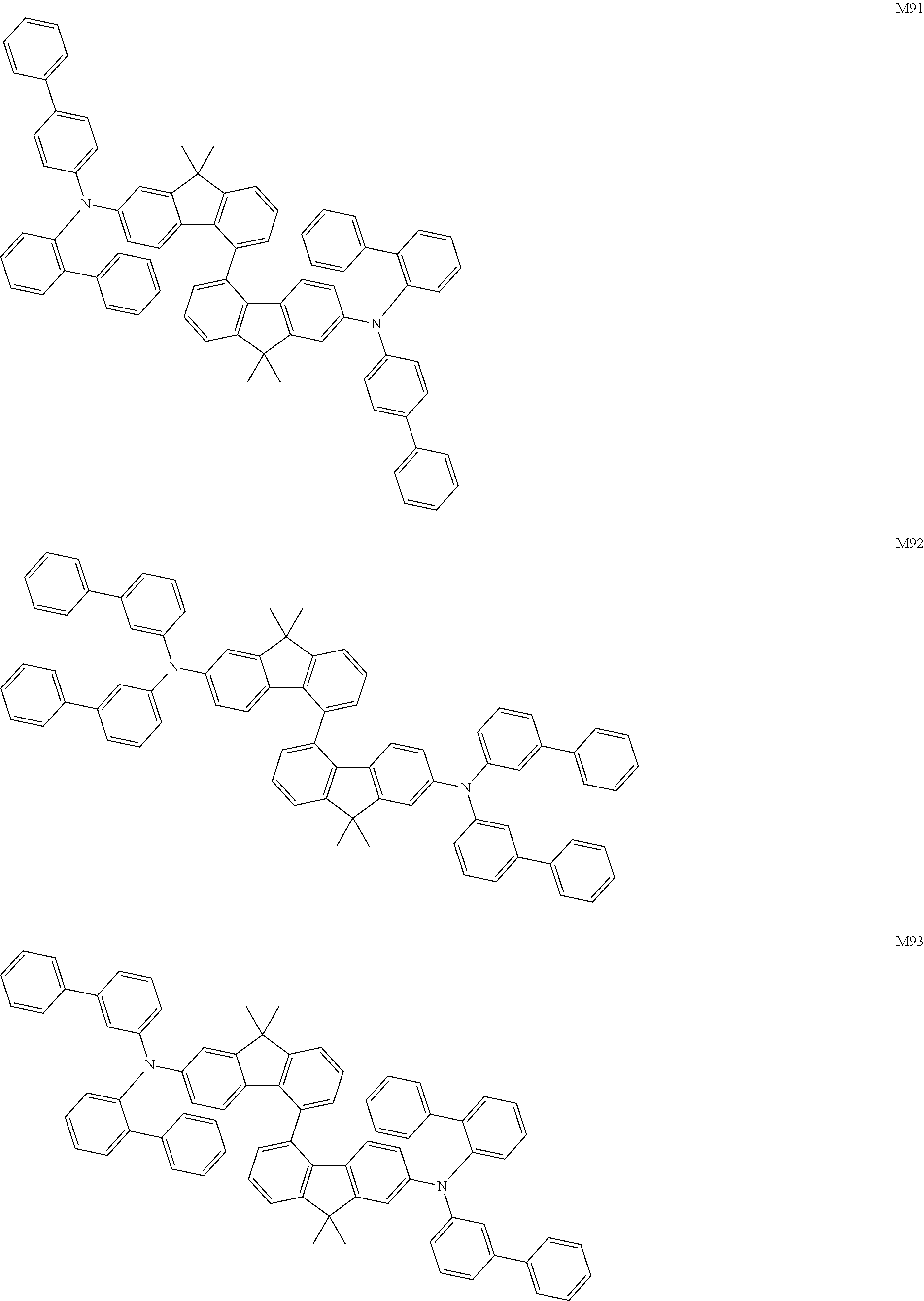 [SMILES: CC1(C)c2cc(N(c3ccc(-c4ccccc4)cc3)c3ccccc3-c3ccccc3)ccc2-c2c(-c3cccc4c3-c3ccc(N(c5ccc(-c6ccccc6)cc5)c5ccccc5-c5ccccc5)cc3C4(C)C)cccc21 ; CC1(C)c2cc(N(c3cccc(-c4ccccc4)c3)c3cccc(-c4ccccc4)c3)ccc2-c2c(-c3cccc4c3-c3ccc(N(c5cccc(-c6ccccc6)c5)c5cccc(-c6ccccc6)c5)cc3C4(C)C)cccc21 ; CC1(C)c2cc(N(c3cccc(-c4ccccc4)c3)c3ccccc3-c3ccccc3)ccc2-c2c(-c3cccc4c3-c3ccc(N(c5cccc(-c6ccccc6)c5)c5ccccc5-c5ccccc5)cc3C4(C)C)cccc21]

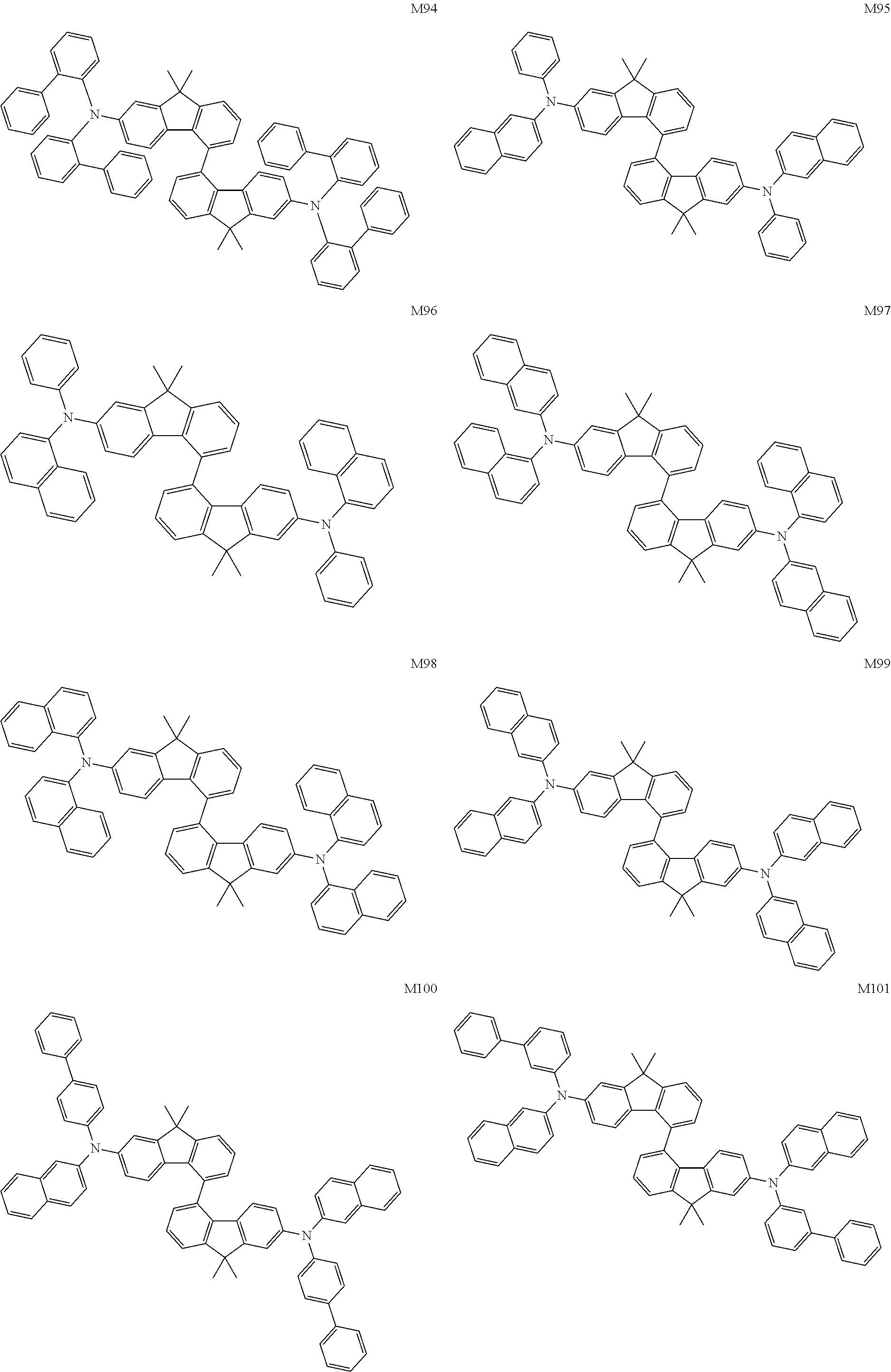

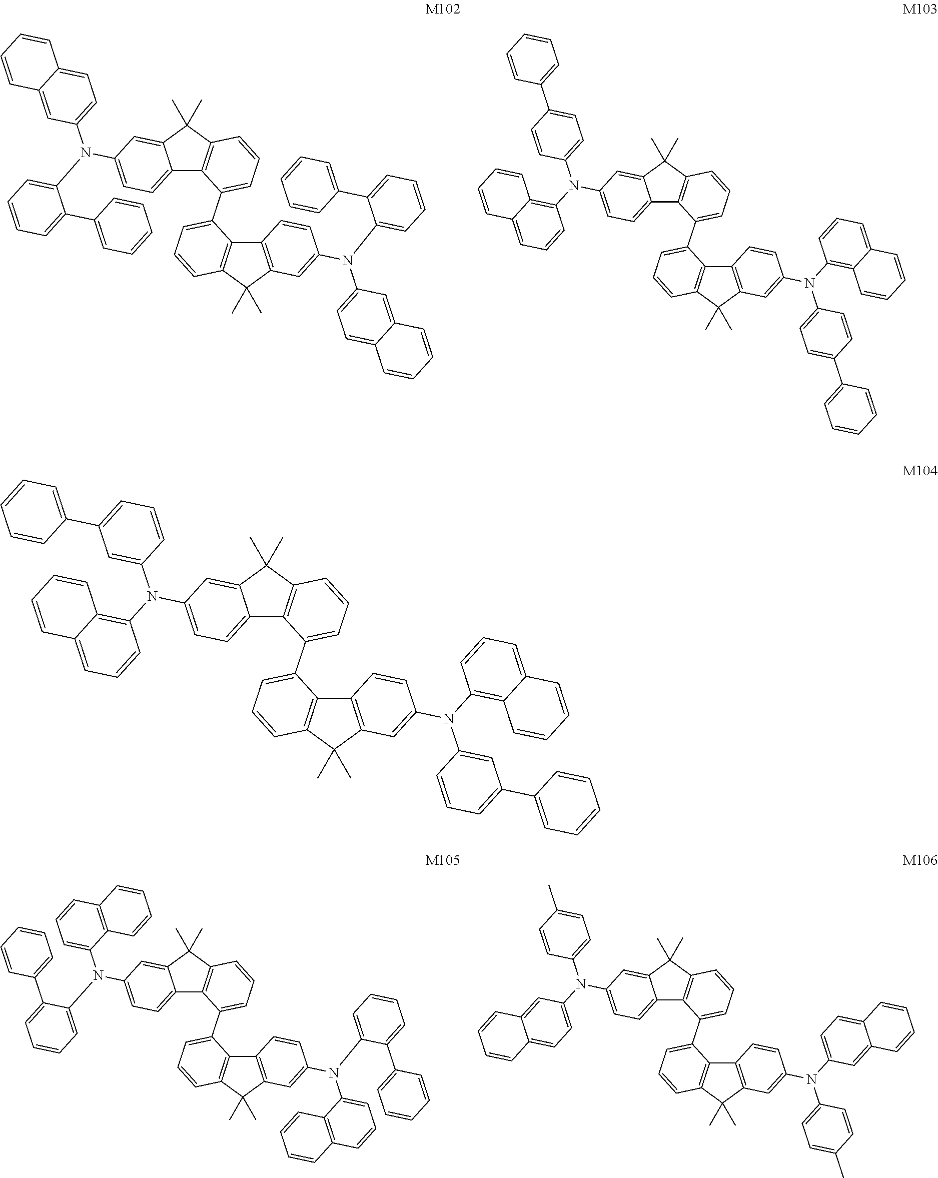

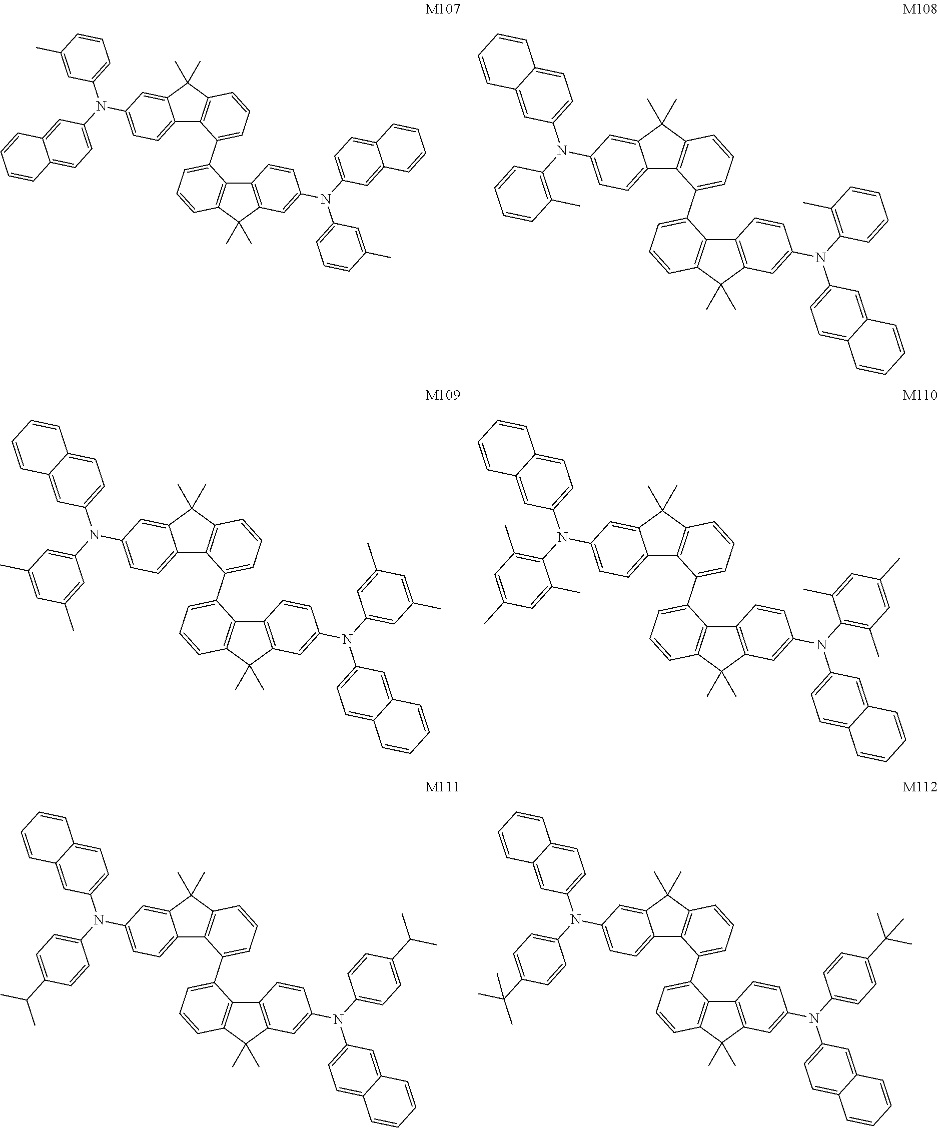

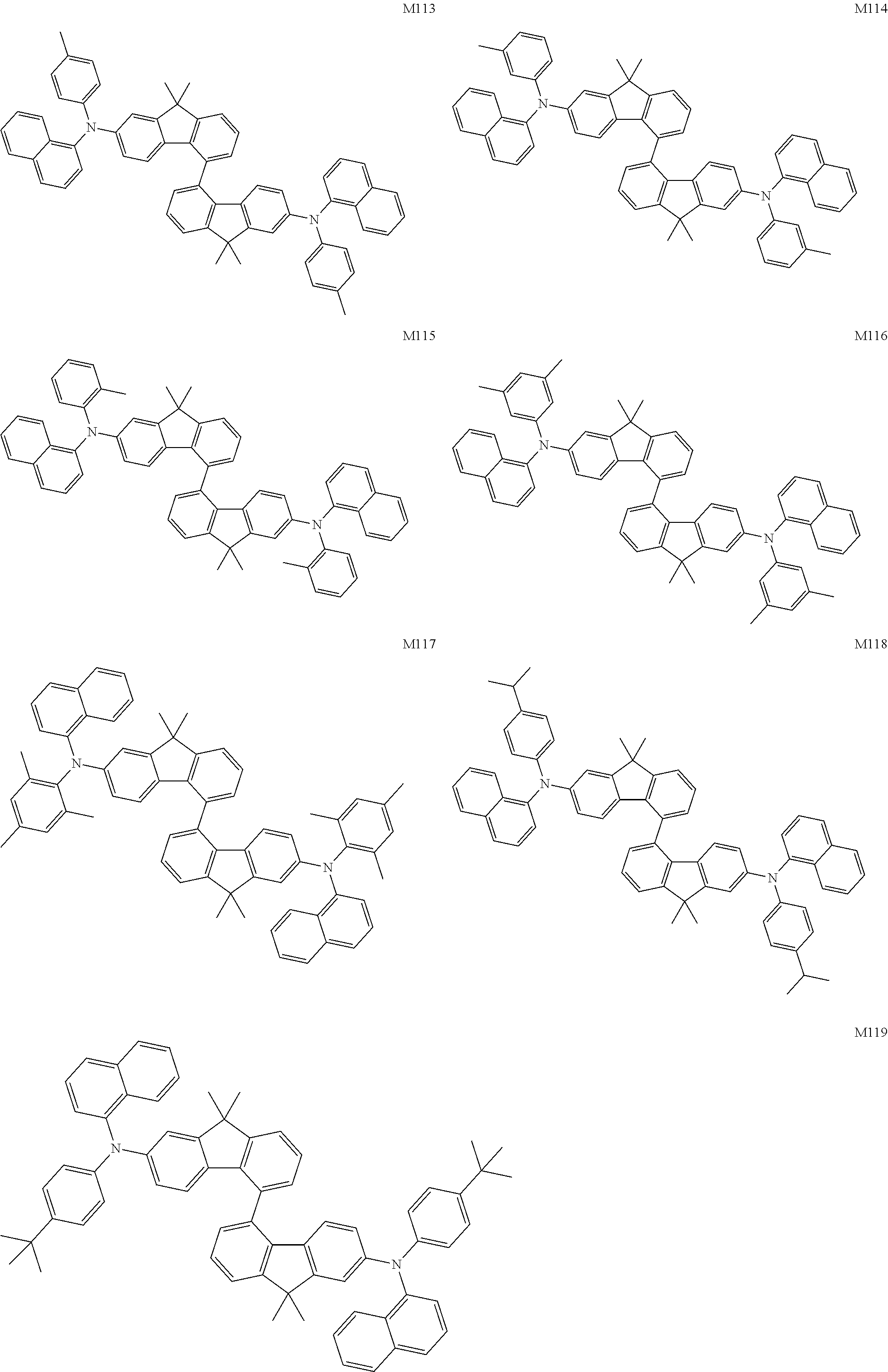

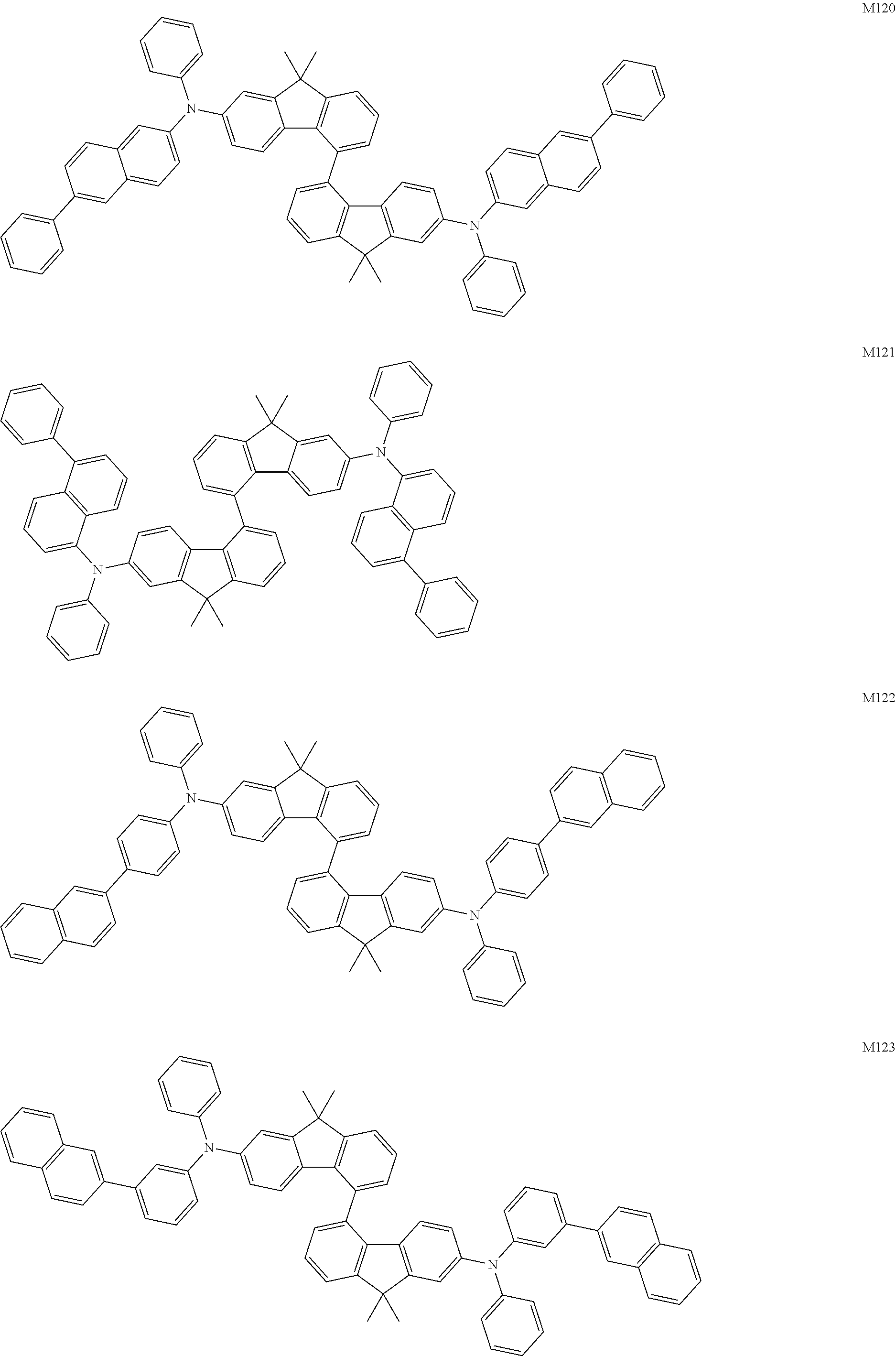

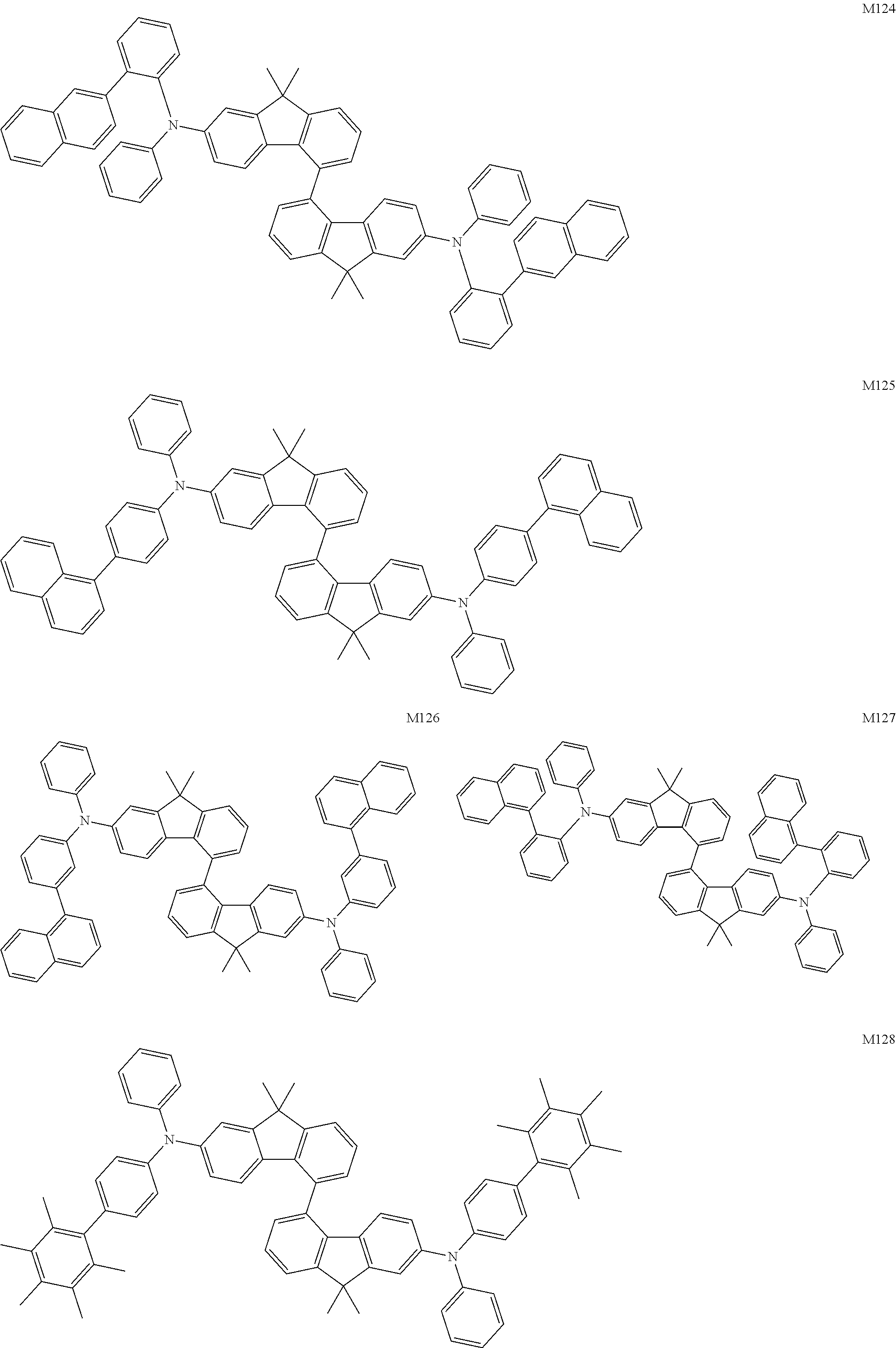

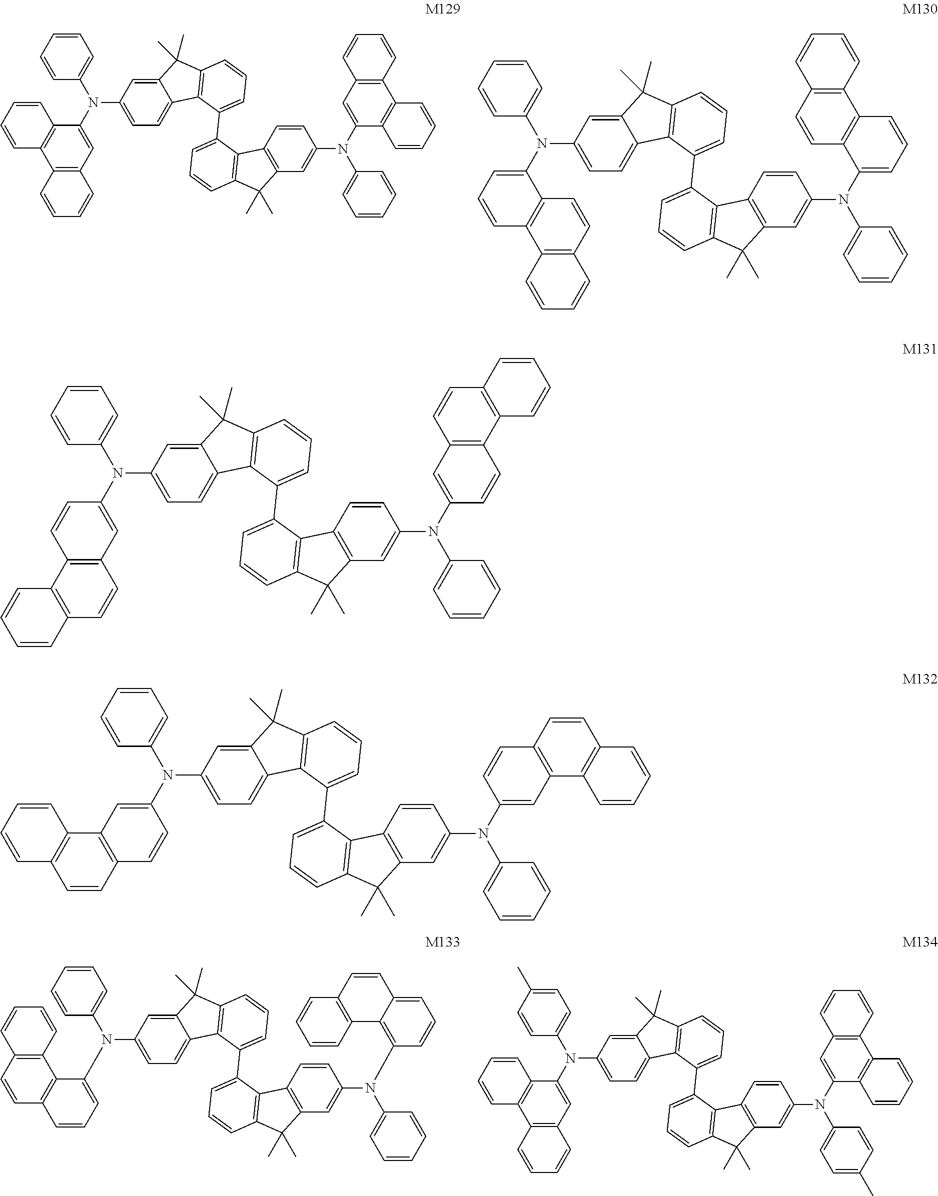

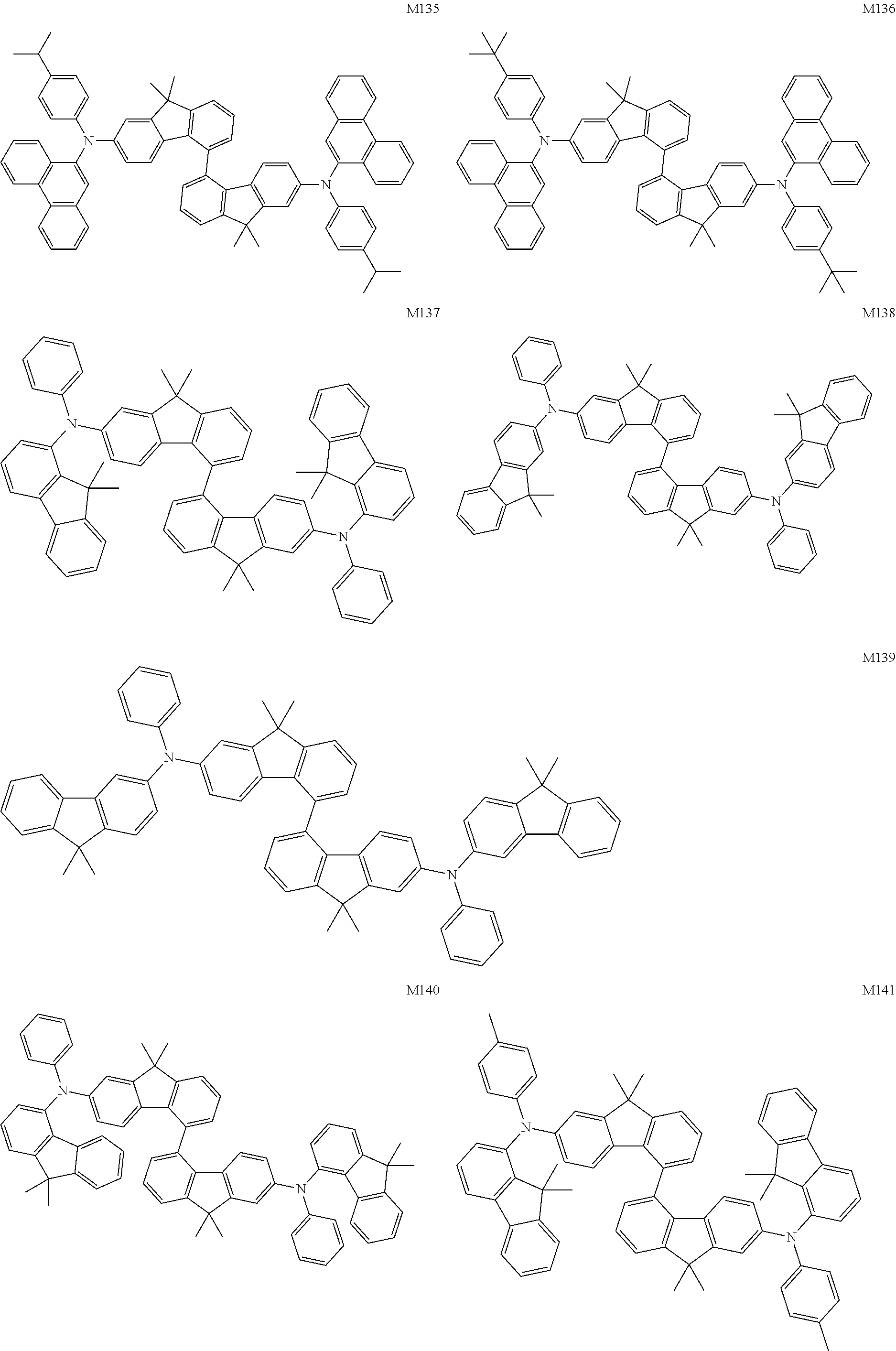

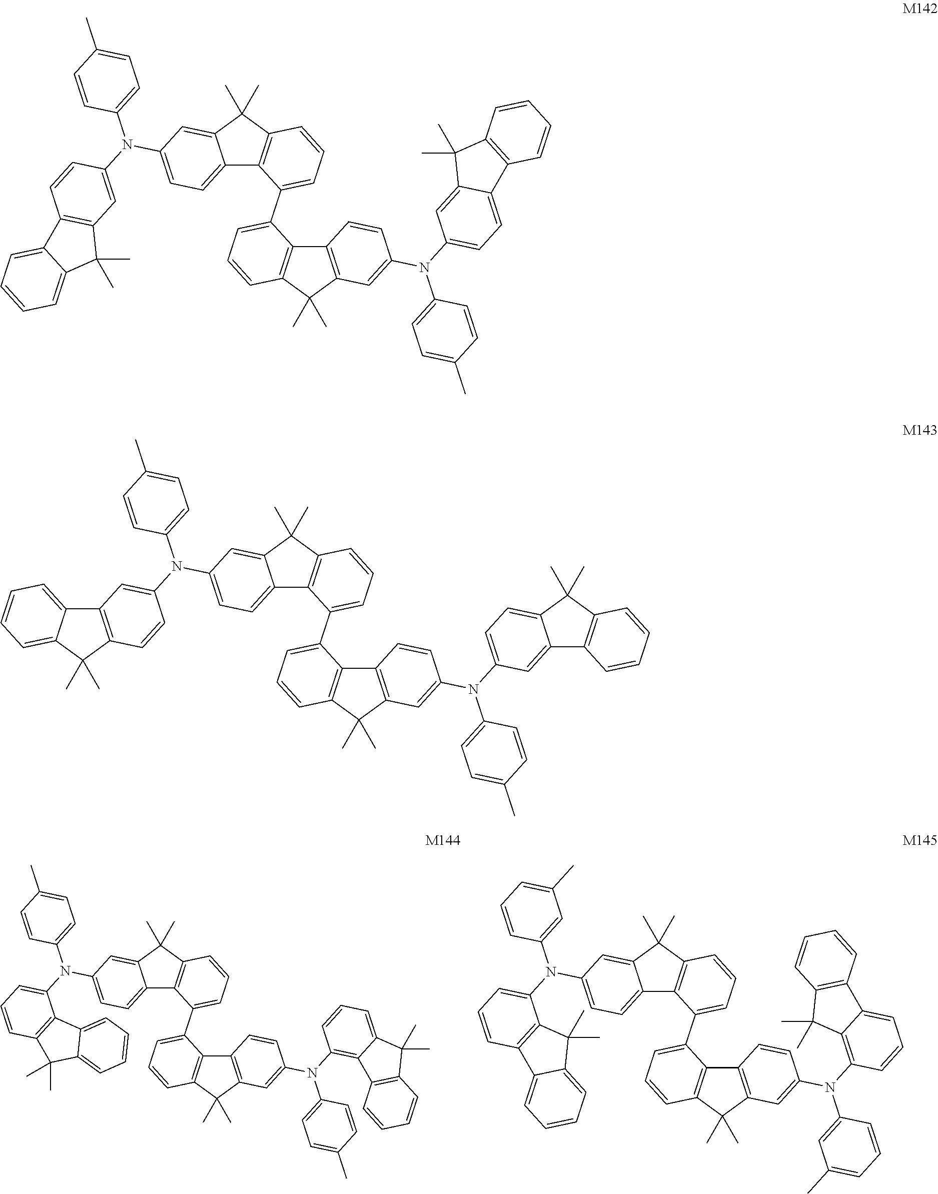

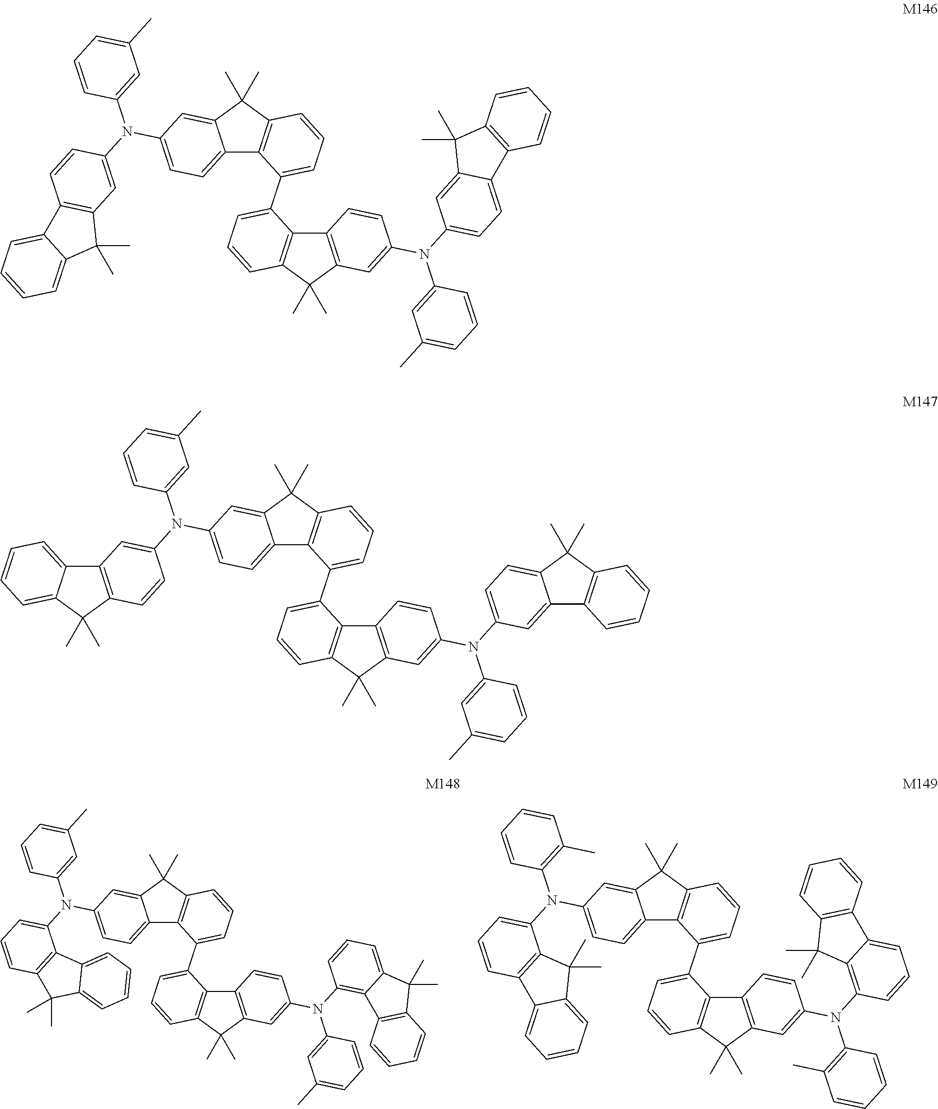

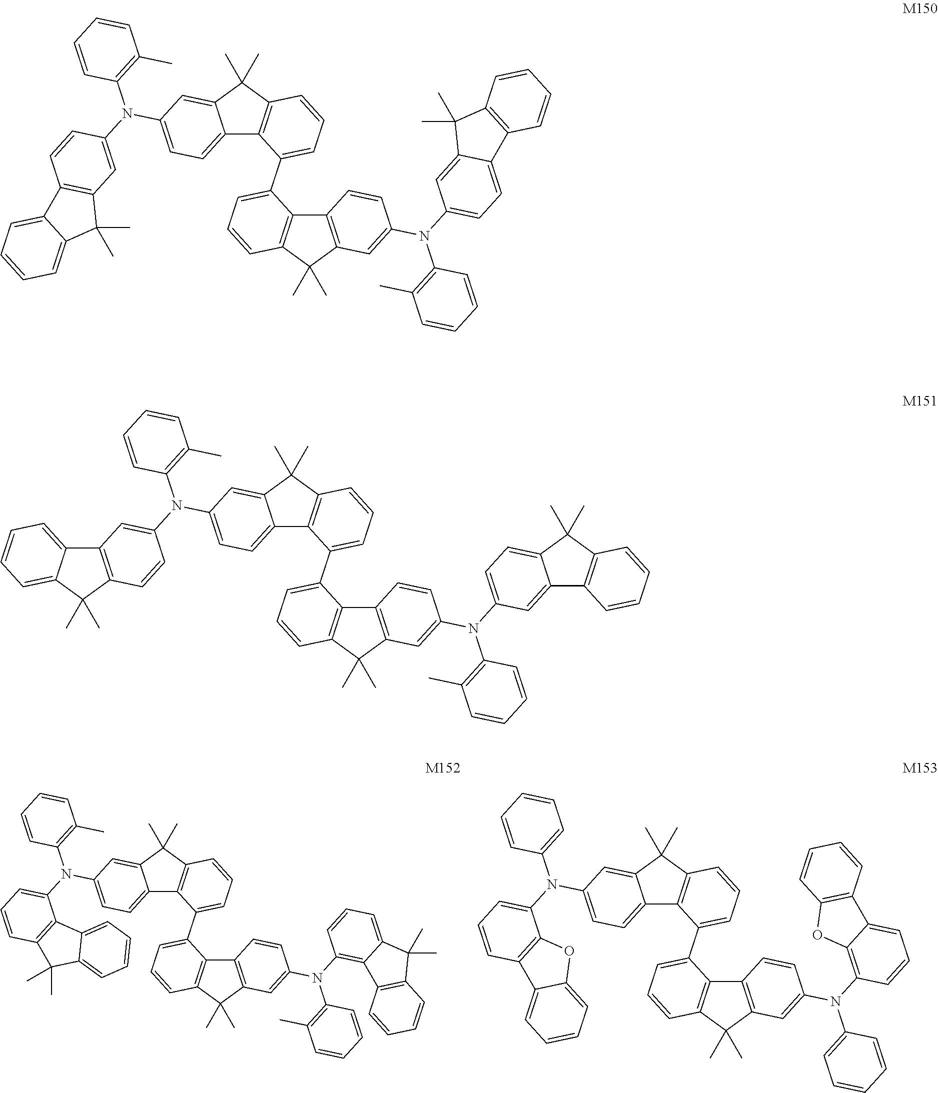

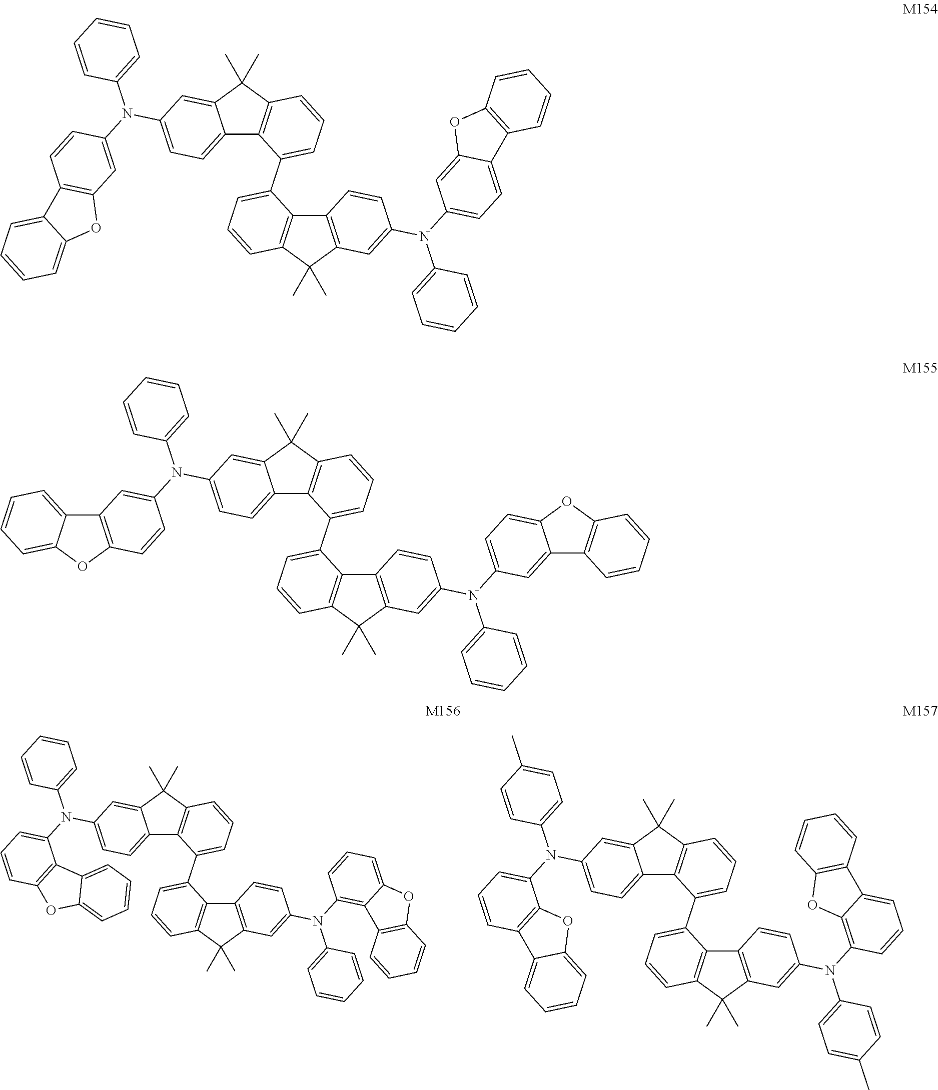 [SMILES: CC1(C)c2cc(N(c3ccccc3)c3ccc4c(c3)oc3ccccc34)ccc2-c2c(-c3cccc4c3-c3ccc(N(c5ccccc5)c5ccc6c(c5)oc5ccccc56)cc3C4(C)C)cccc21 ; CC1(C)c2cc(N(c3ccccc3)c3ccc4oc5ccccc5c4c3)ccc2-c2c(-c3cccc4c3-c3ccc(N(c5ccccc5)c5ccc6oc7ccccc7c6c5)cc3C4(C)C)cccc21 ; CC1(C)c2cc(N(c3ccccc3)c3cccc4oc5ccccc5c34)ccc2-c2c(-c3cccc4c3-c3ccc(N(c5ccccc5)c5cccc6oc7ccccc7c56)cc3C4(C)C)cccc21 ; Cc1ccc(N(c2ccc3c(c2)C(C)(C)c2cccc(-c4cccc5c4-c4ccc(N(c6ccc(C)cc6)c6cccc7c6oc6ccccc67)cc4C5(C)C)c2-3)c2cccc3c2oc2ccccc23)cc1]

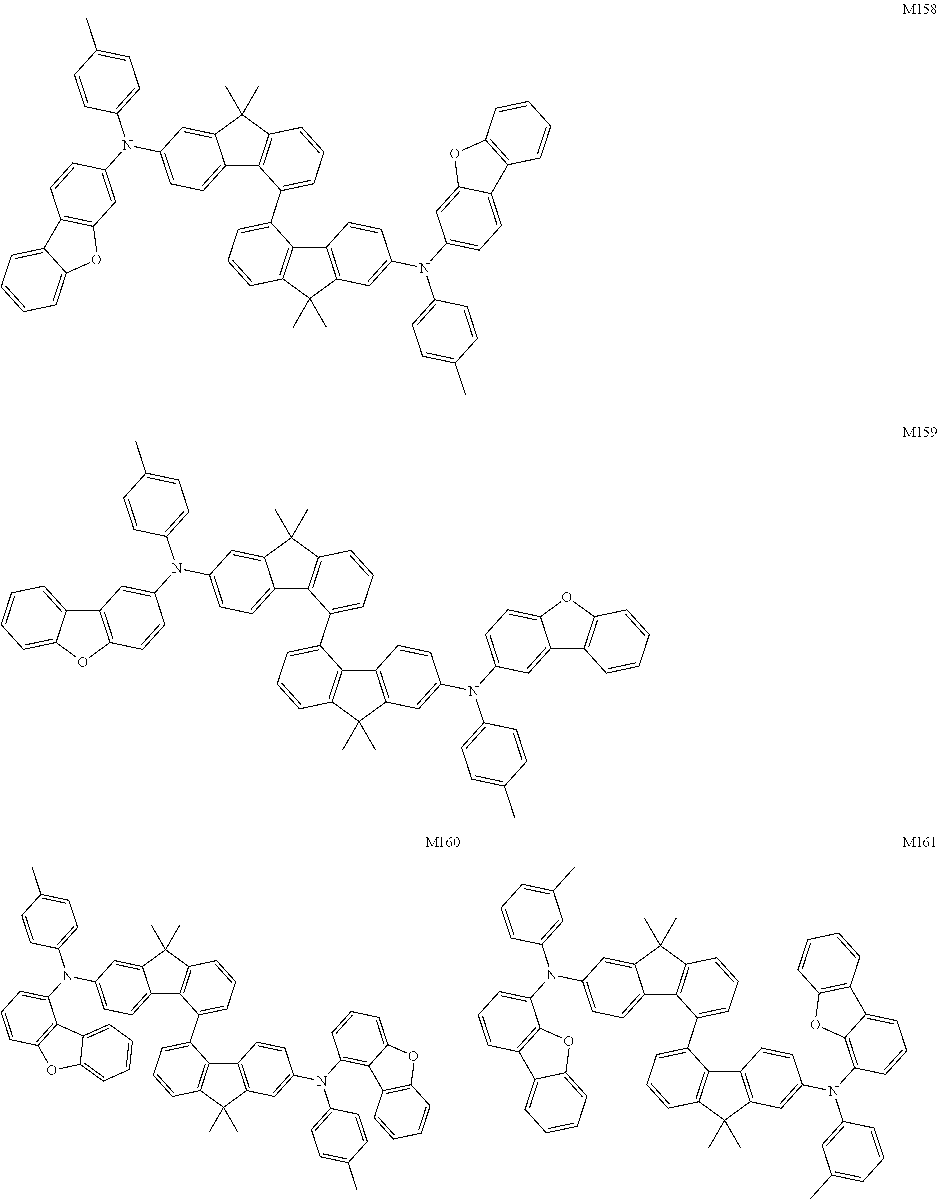

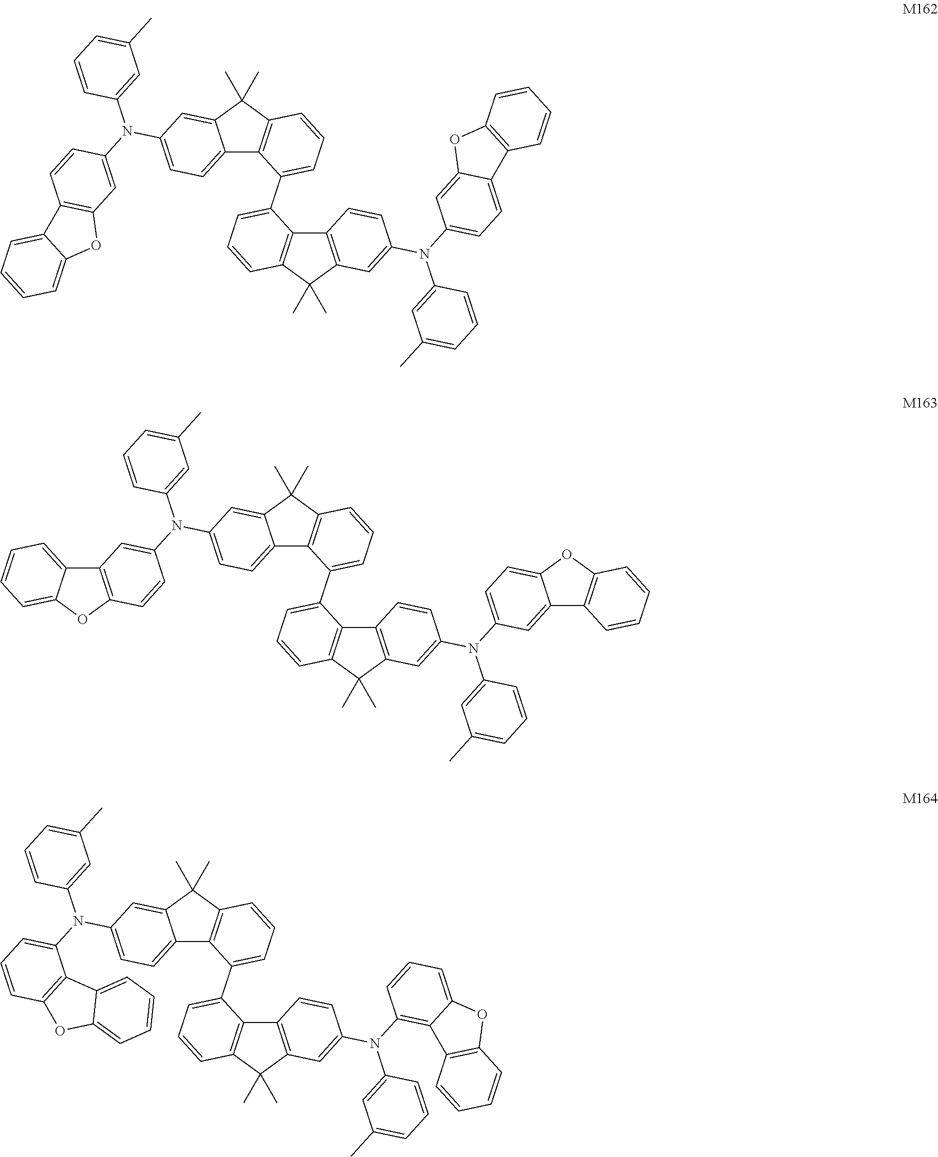 [SMILES: Cc1cccc(N(c2ccc3c(c2)C(C)(C)c2cccc(-c4cccc5c4-c4ccc(N(c6cccc(C)c6)c6ccc7c(c6)oc6ccccc67)cc4C5(C)C)c2-3)c2ccc3c(c2)oc2ccccc23)c1 ; Cc1cccc(N(c2ccc3c(c2)C(C)(C)c2cccc(-c4cccc5c4-c4ccc(N(c6cccc(C)c6)c6ccc7oc8ccccc8c7c6)cc4C5(C)C)c2-3)c2ccc3oc4ccccc4c3c2)c1 ; Cc1cccc(N(c2ccc3c(c2)C(C)(C)c2cccc(-c4cccc5c4-c4ccc(N(c6cccc(C)c6)c6cccc7oc8ccccc8c67)cc4C5(C)C)c2-3)c2cccc3oc4ccccc4c23)c1]

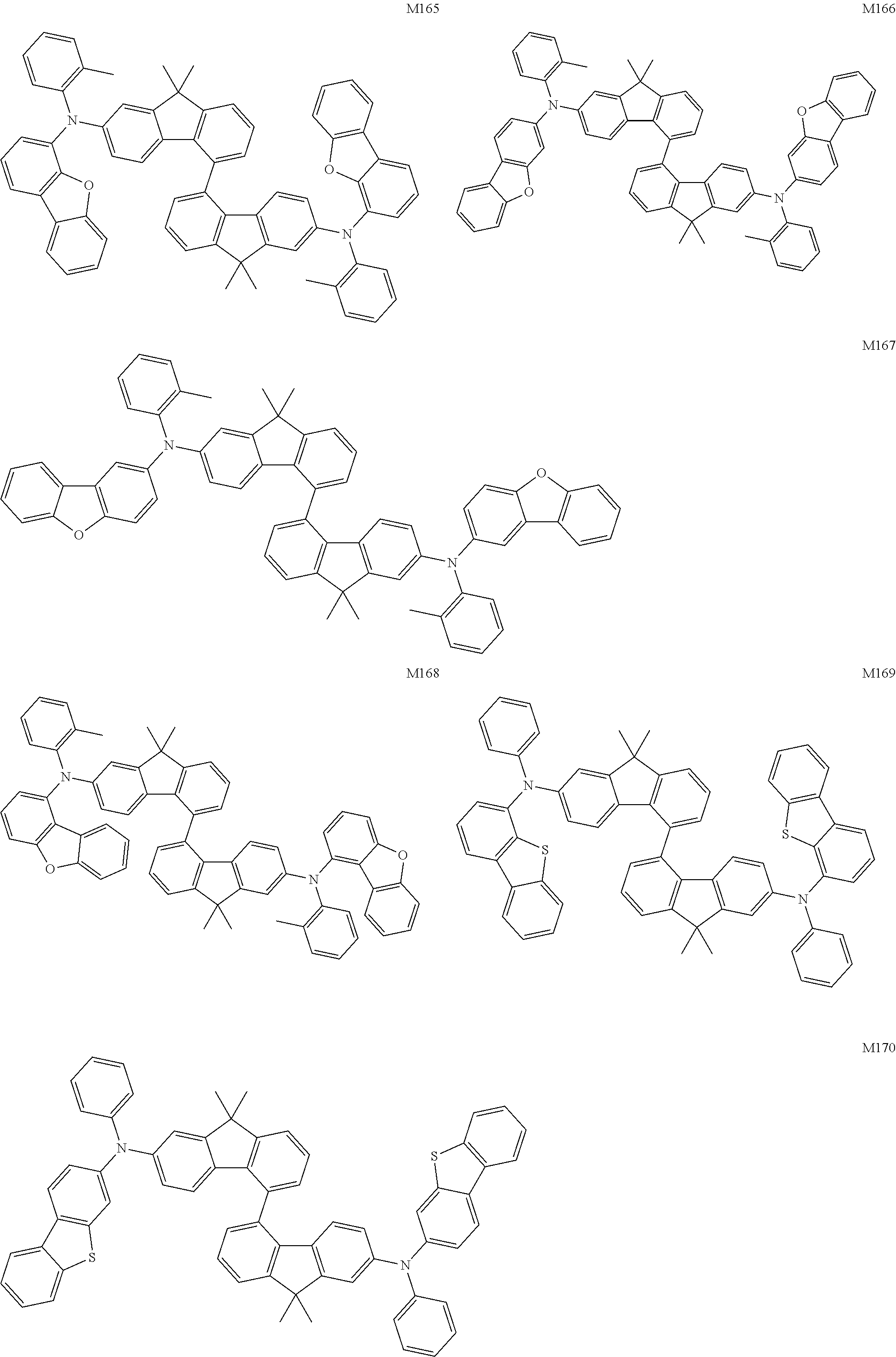

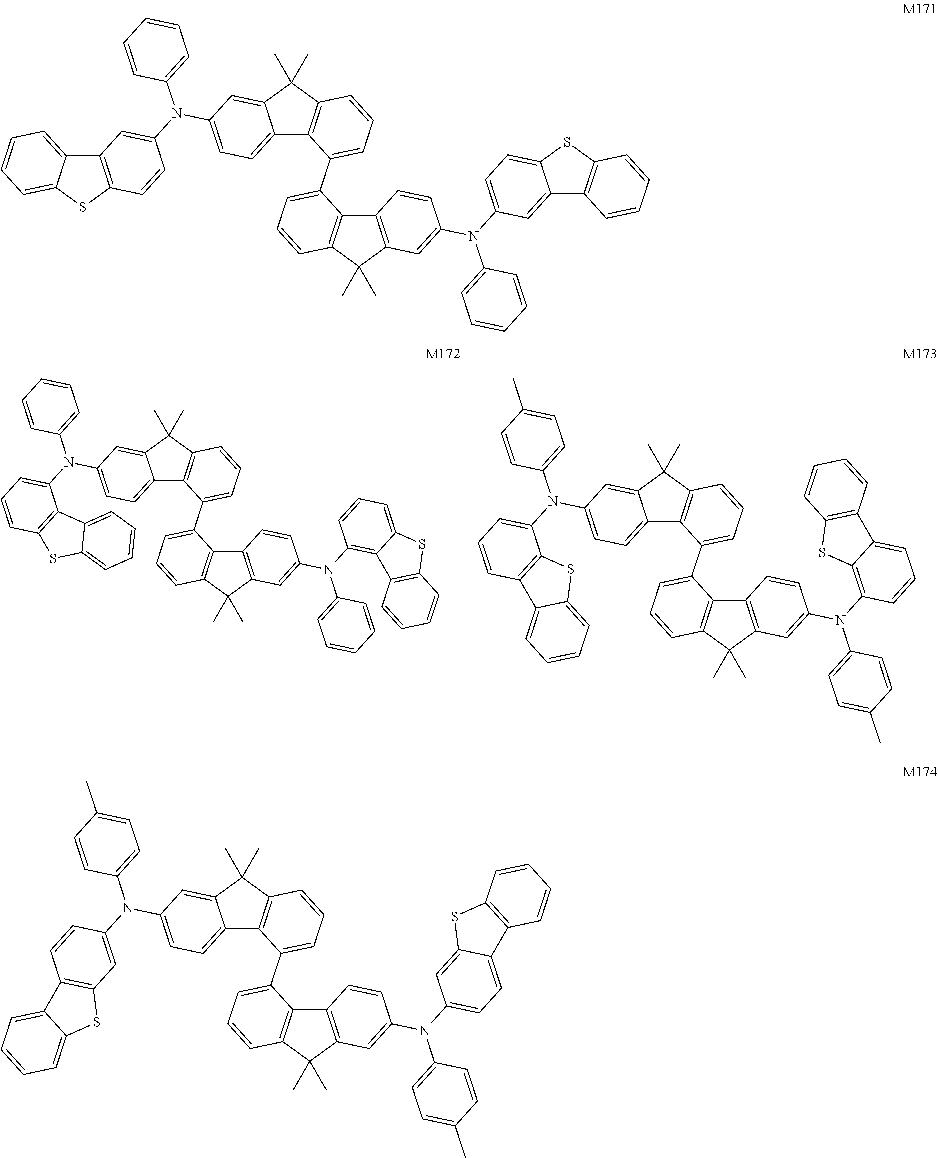 [SMILES: CC1(C)c2cc(N(c3ccccc3)c3ccc4sc5ccccc5c4c3)ccc2-c2c(-c3cccc4c3-c3ccc(N(c5ccccc5)c5ccc6sc7ccccc7c6c5)cc3C4(C)C)cccc21 ; CC1(C)c2cc(N(c3ccccc3)c3cccc4sc5ccccc5c34)ccc2-c2c(-c3cccc4c3-c3ccc(N(c5ccccc5)c5cccc6sc7ccccc7c56)cc3C4(C)C)cccc21 ; Cc1ccc(N(c2ccc3c(c2)C(C)(C)c2cccc(-c4cccc5c4-c4ccc(N(c6ccc(C)cc6)c6cccc7c6sc6ccccc67)cc4C5(C)C)c2-3)c2cccc3c2sc2ccccc23)cc1 ; Cc1ccc(N(c2ccc3c(c2)C(C)(C)c2cccc(-c4cccc5c4-c4ccc(N(c6ccc(C)cc6)c6ccc7c(c6)sc6ccccc67)cc4C5(C)C)c2-3)c2ccc3c(c2)sc2ccccc23)cc1]

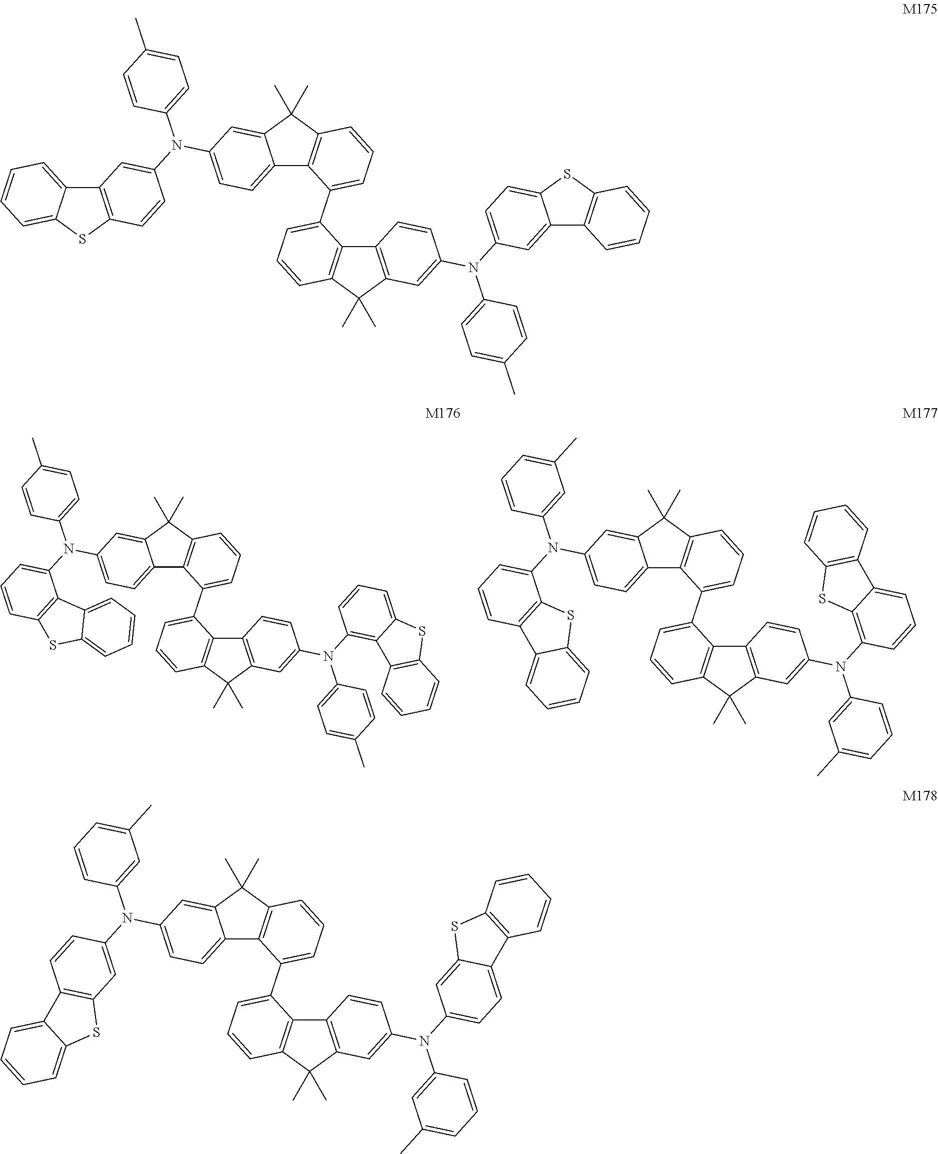

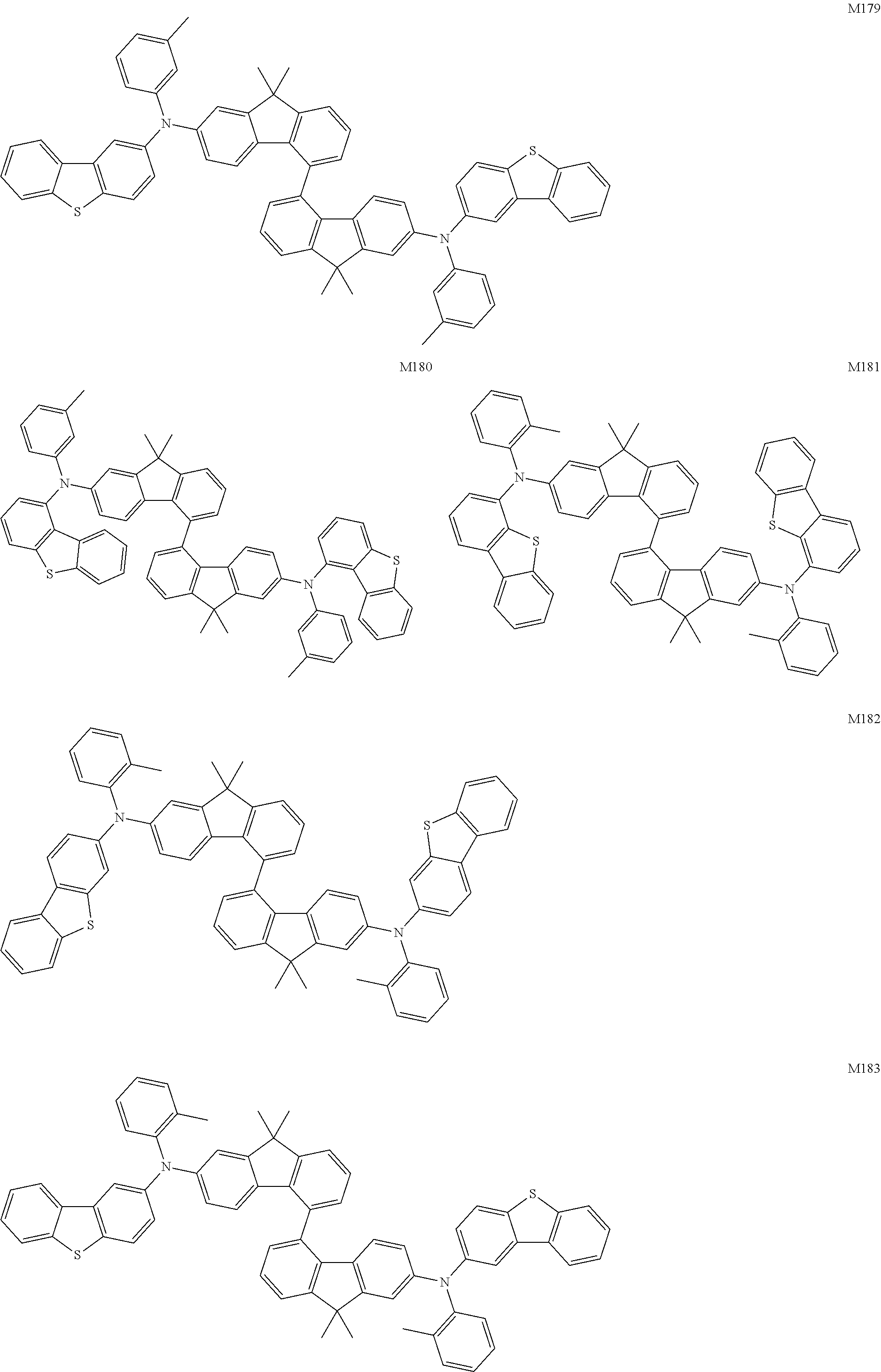

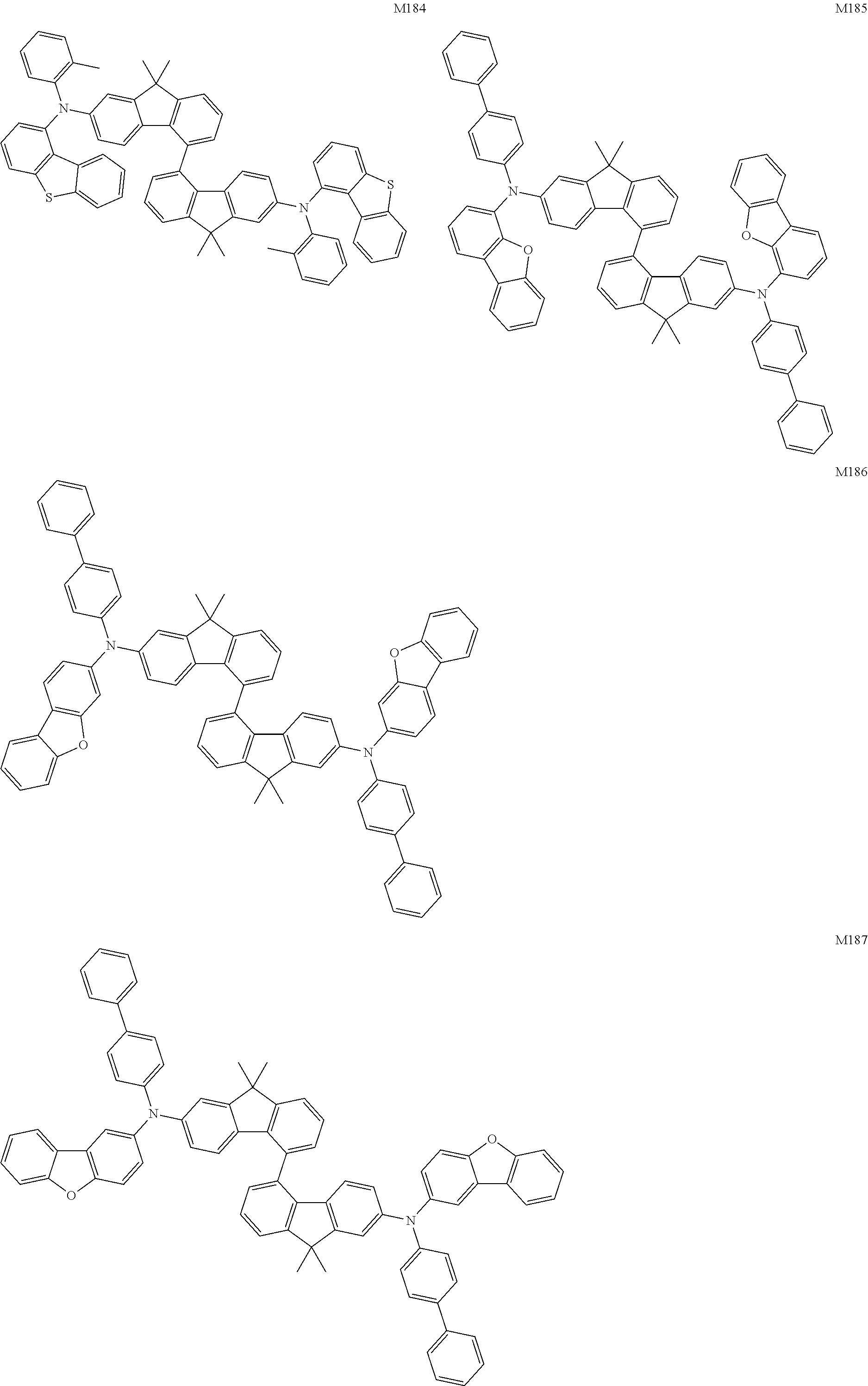

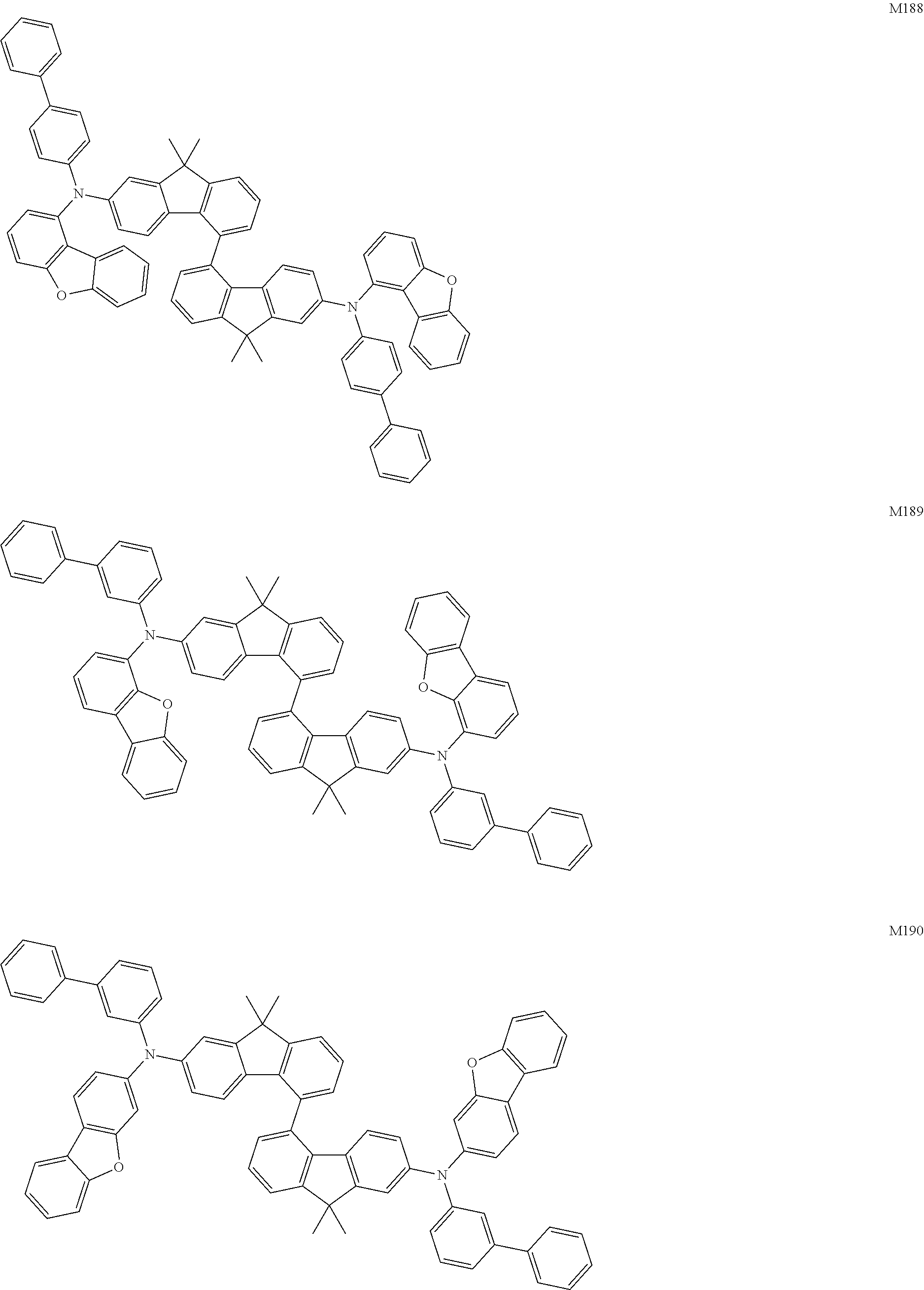 [SMILES: CC1(C)c2cc(N(c3ccc(-c4ccccc4)cc3)c3cccc4oc5ccccc5c34)ccc2-c2c(-c3cccc4c3-c3ccc(N(c5ccc(-c6ccccc6)cc5)c5cccc6oc7ccccc7c56)cc3C4(C)C)cccc21 ; CC1(C)c2cc(N(c3cccc(-c4ccccc4)c3)c3cccc4c3oc3ccccc34)ccc2-c2c(-c3cccc4c3-c3ccc(N(c5cccc(-c6ccccc6)c5)c5cccc6c5oc5ccccc56)cc3C4(C)C)cccc21 ; CC1(C)c2cc(N(c3cccc(-c4ccccc4)c3)c3ccc4c(c3)oc3ccccc34)ccc2-c2c(-c3cccc4c3-c3ccc(N(c5cccc(-c6ccccc6)c5)c5ccc6c(c5)oc5ccccc56)cc3C4(C)C)cccc21]

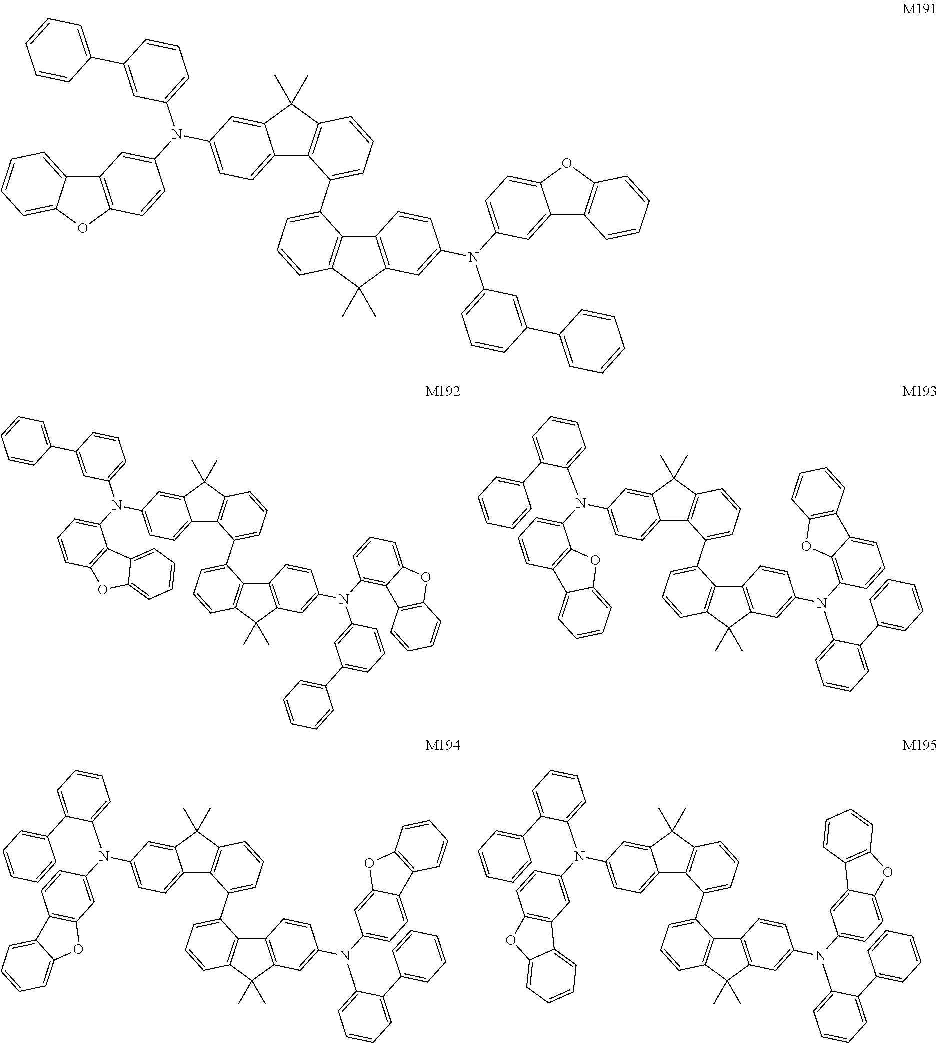

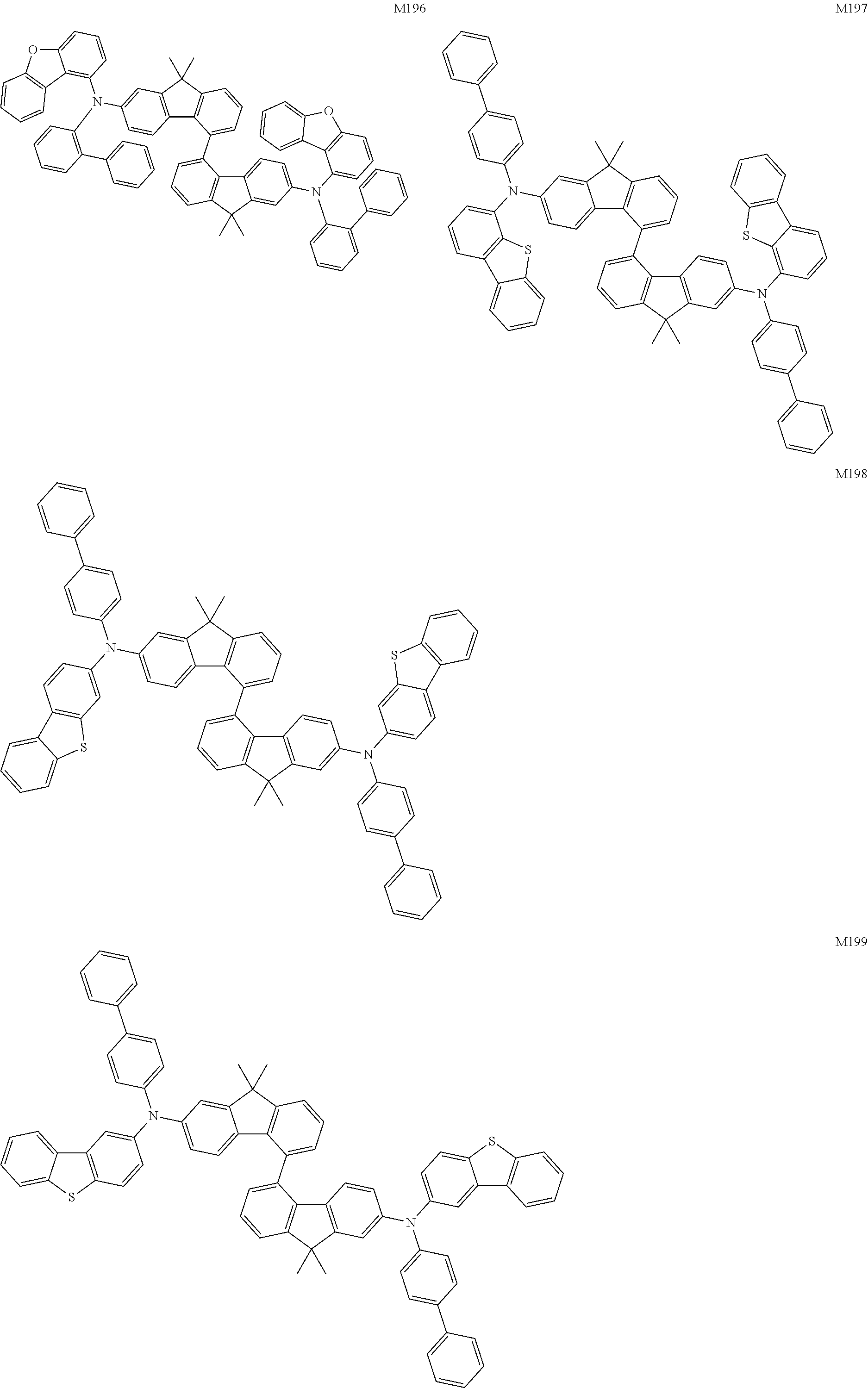

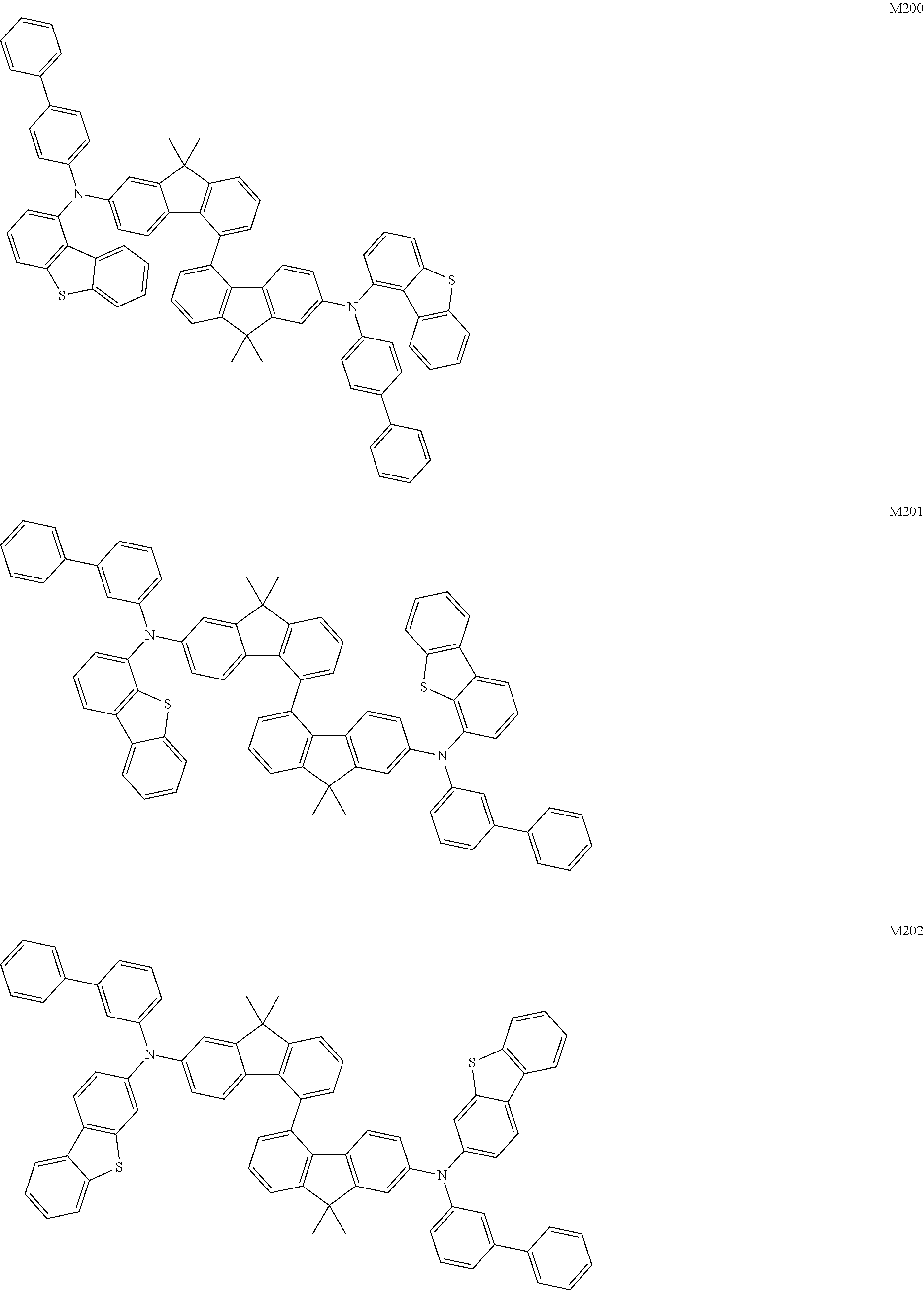 [SMILES: CC1(C)c2cc(N(c3ccc(-c4ccccc4)cc3)c3cccc4sc5ccccc5c34)ccc2-c2c(-c3cccc4c3-c3ccc(N(c5ccc(-c6ccccc6)cc5)c5cccc6sc7ccccc7c56)cc3C4(C)C)cccc21 ; CC1(C)c2cc(N(c3cccc(-c4ccccc4)c3)c3cccc4c3sc3ccccc34)ccc2-c2c(-c3cccc4c3-c3ccc(N(c5cccc(-c6ccccc6)c5)c5cccc6c5sc5ccccc56)cc3C4(C)C)cccc21 ; CC1(C)c2cc(N(c3cccc(-c4ccccc4)c3)c3ccc4c(c3)sc3ccccc34)ccc2-c2c(-c3cccc4c3-c3ccc(N(c5cccc(-c6ccccc6)c5)c5ccc6c(c5)sc5ccccc56)cc3C4(C)C)cccc21]

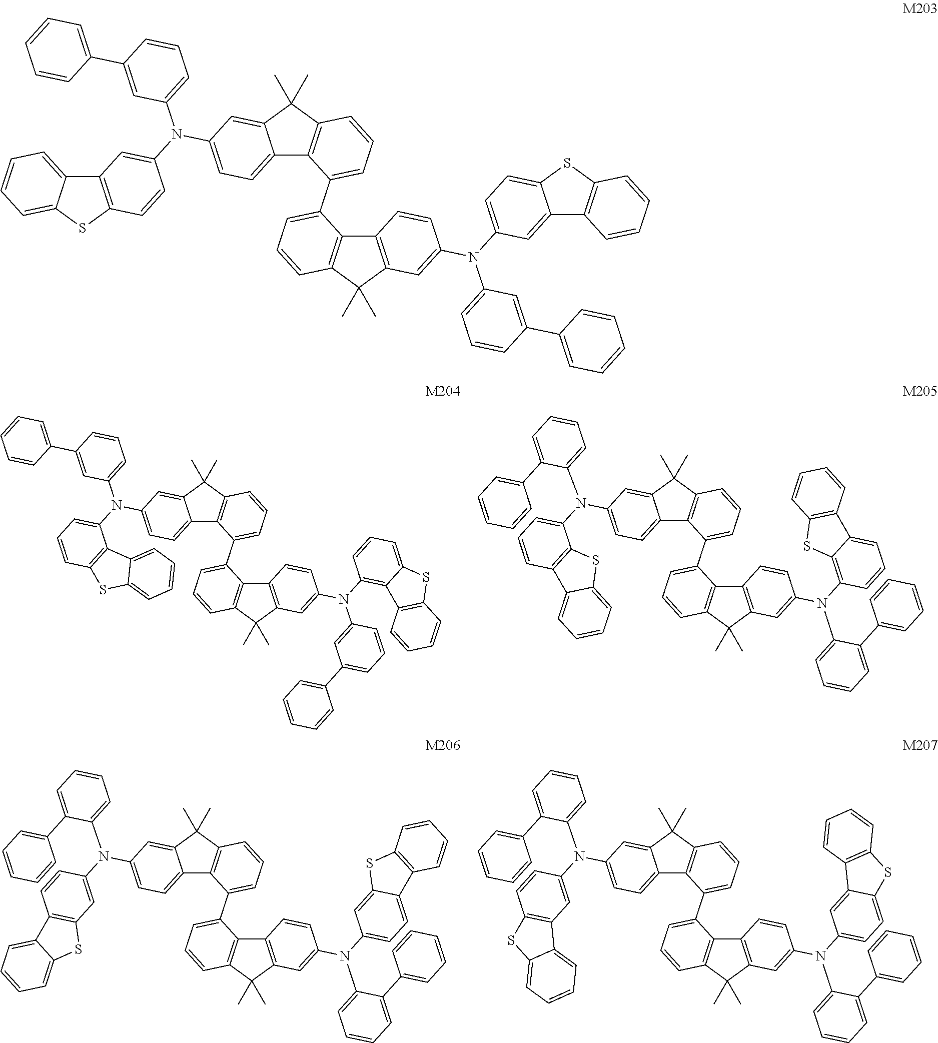

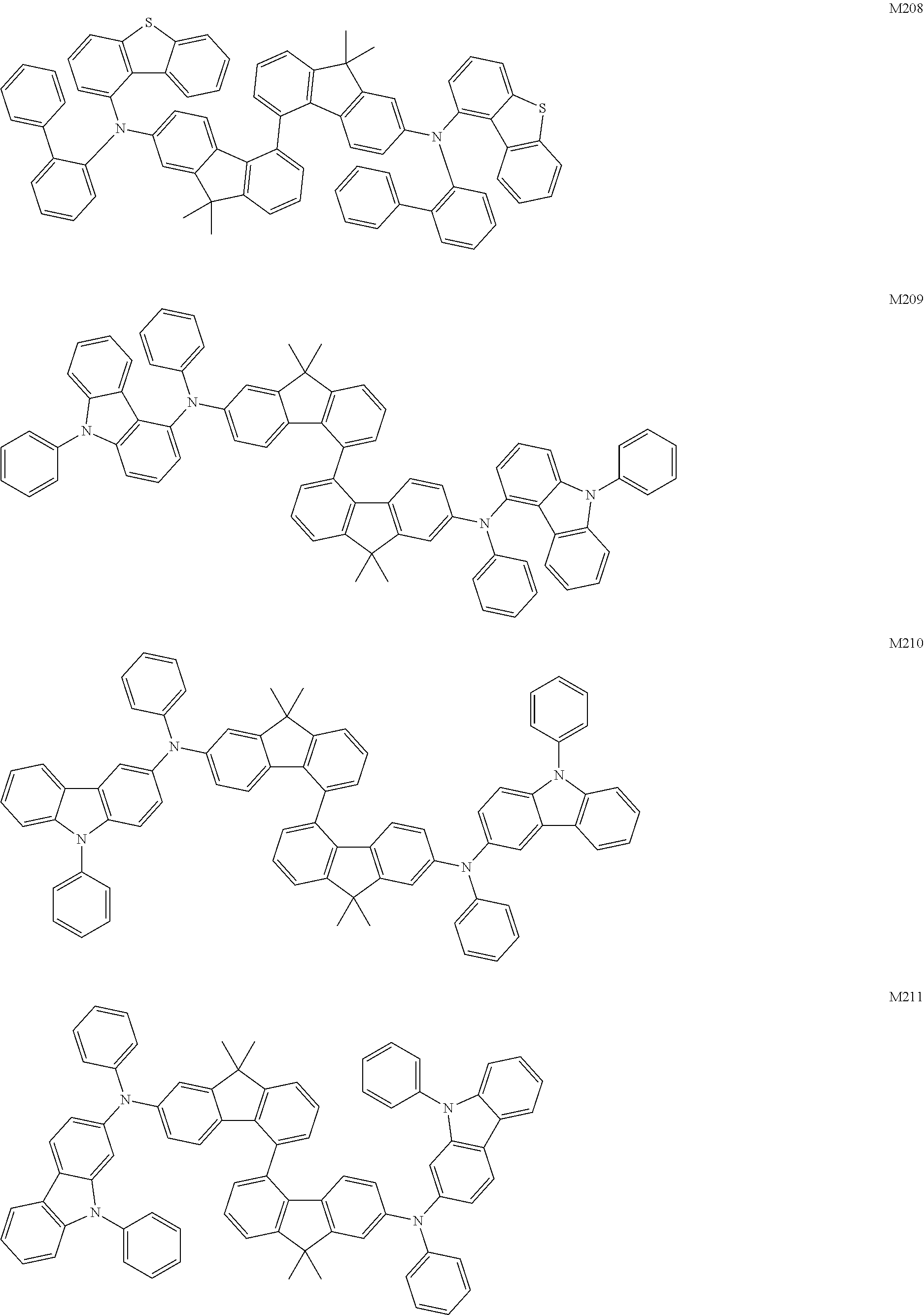

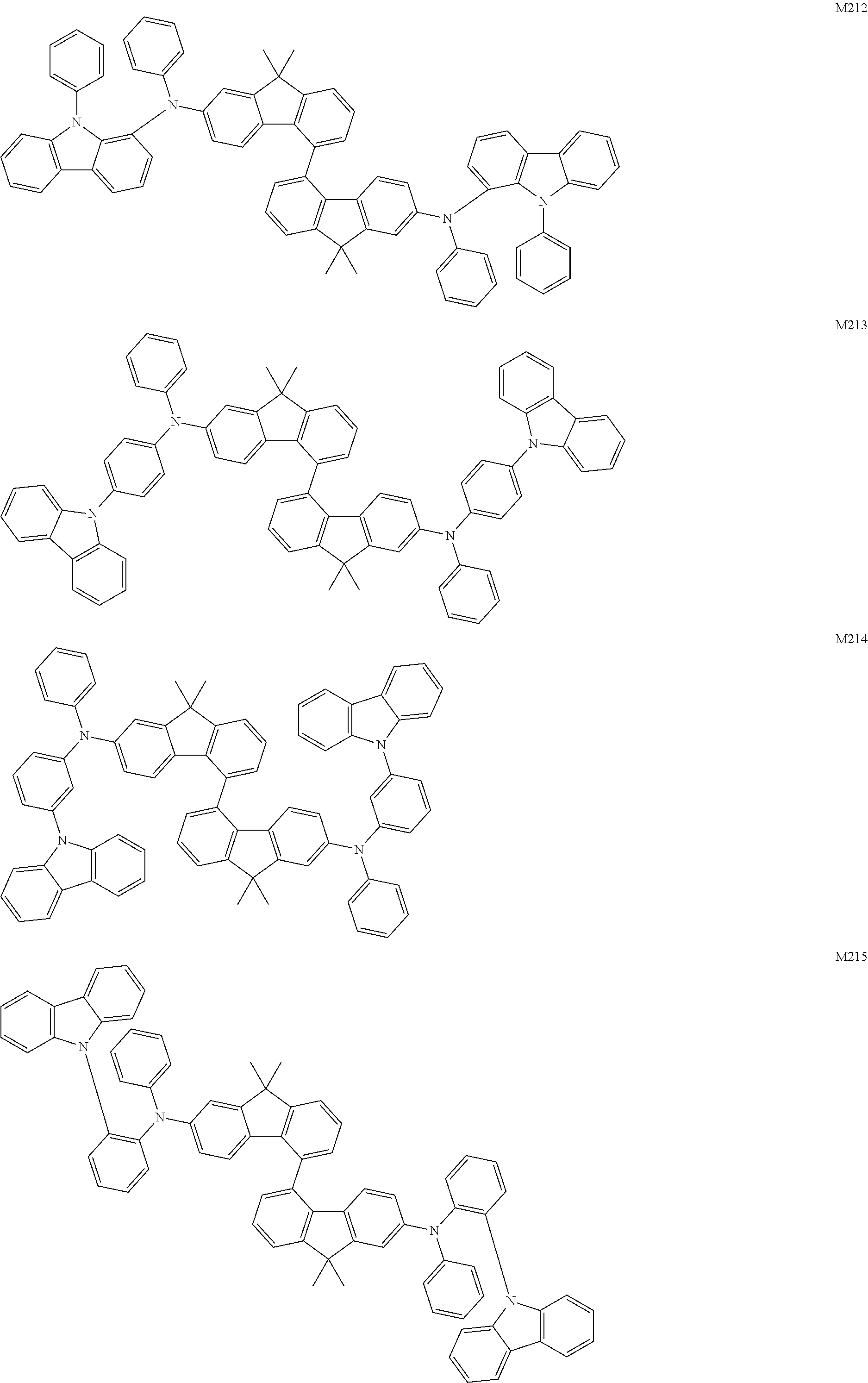

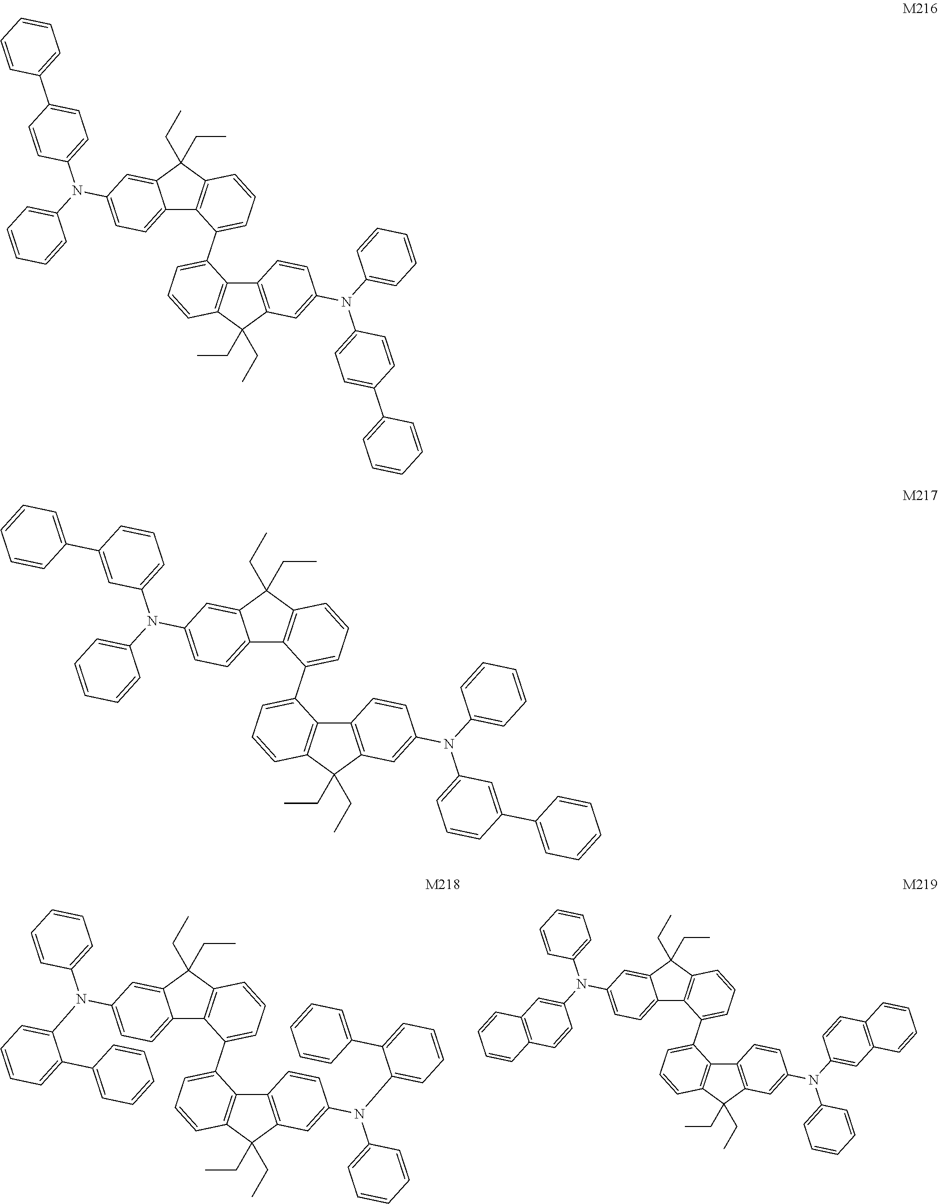

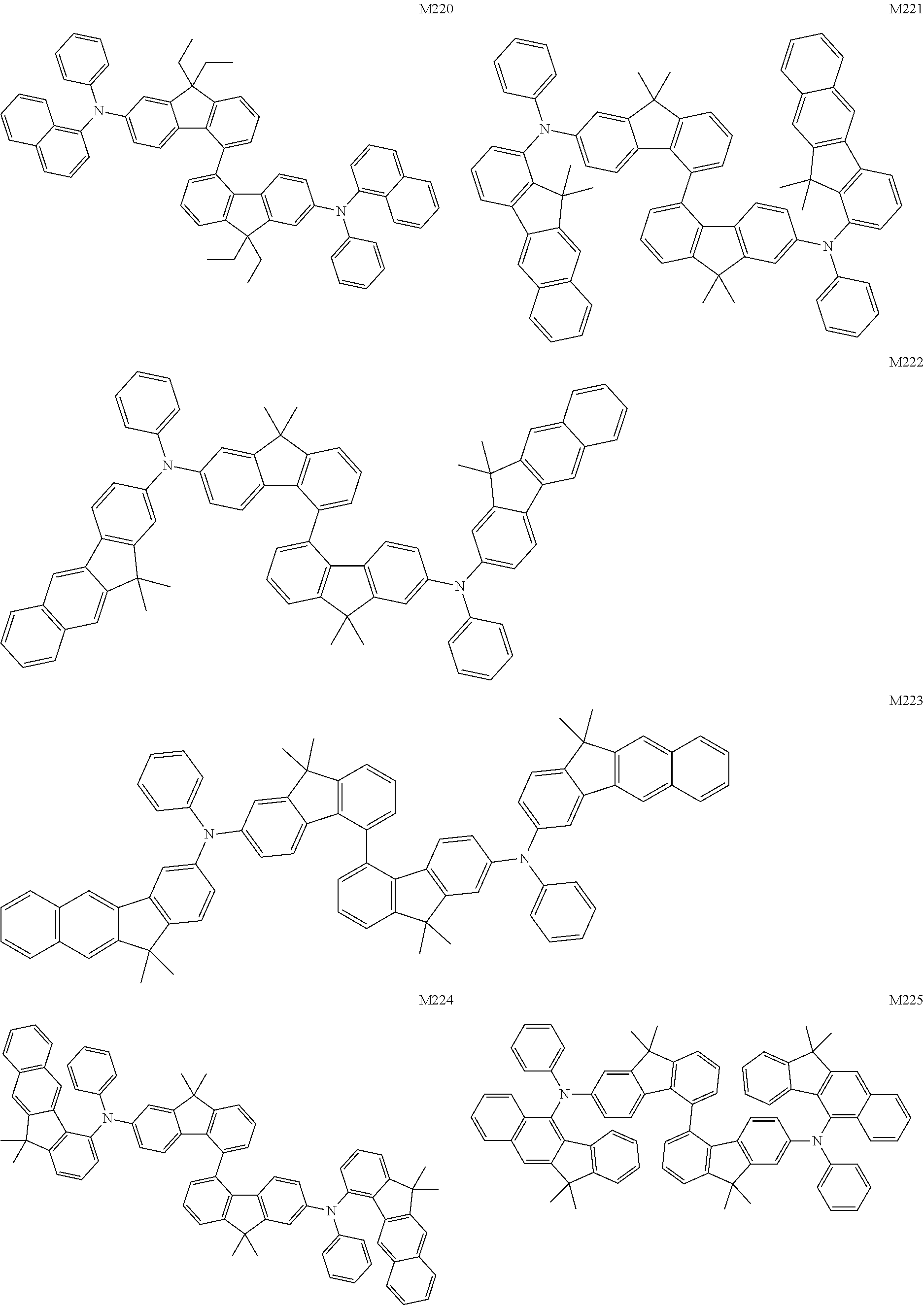

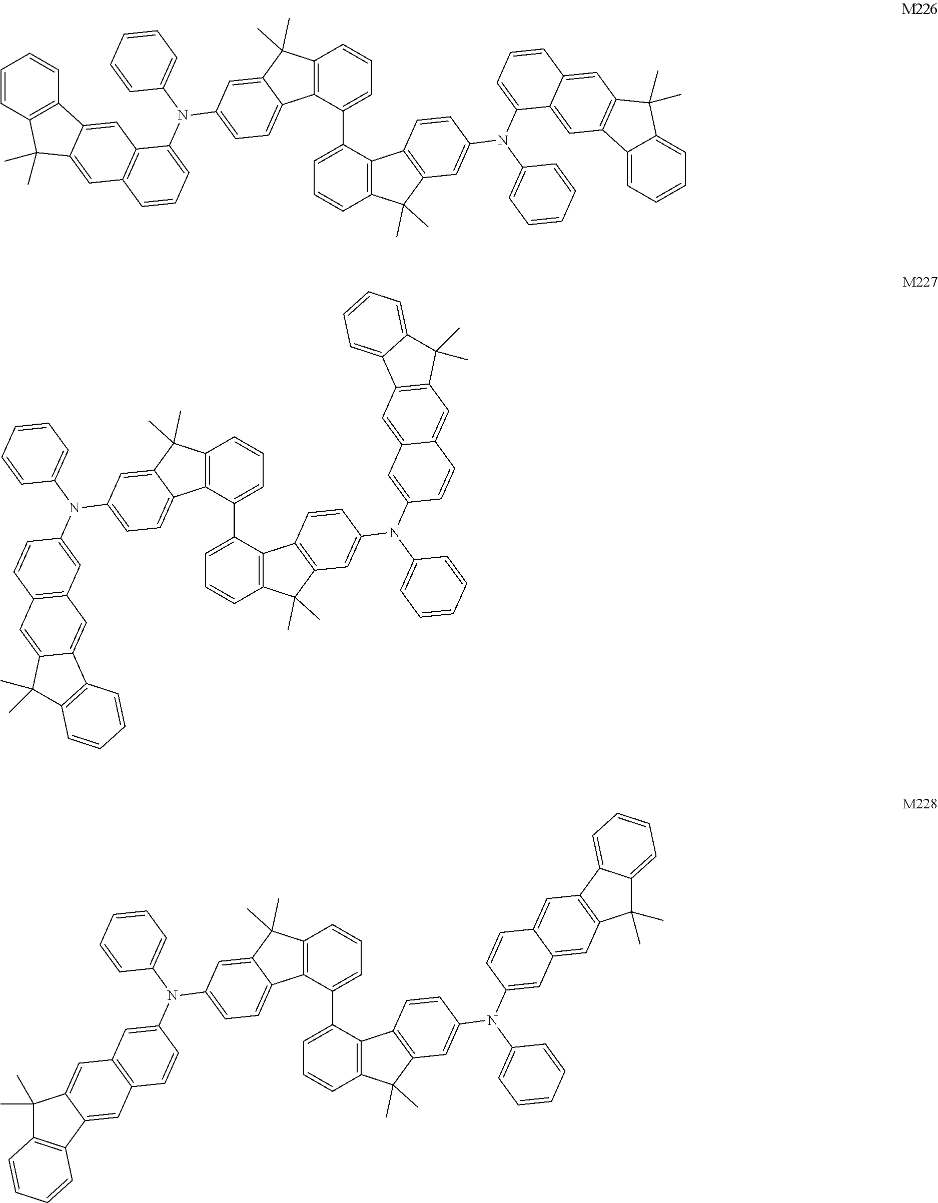

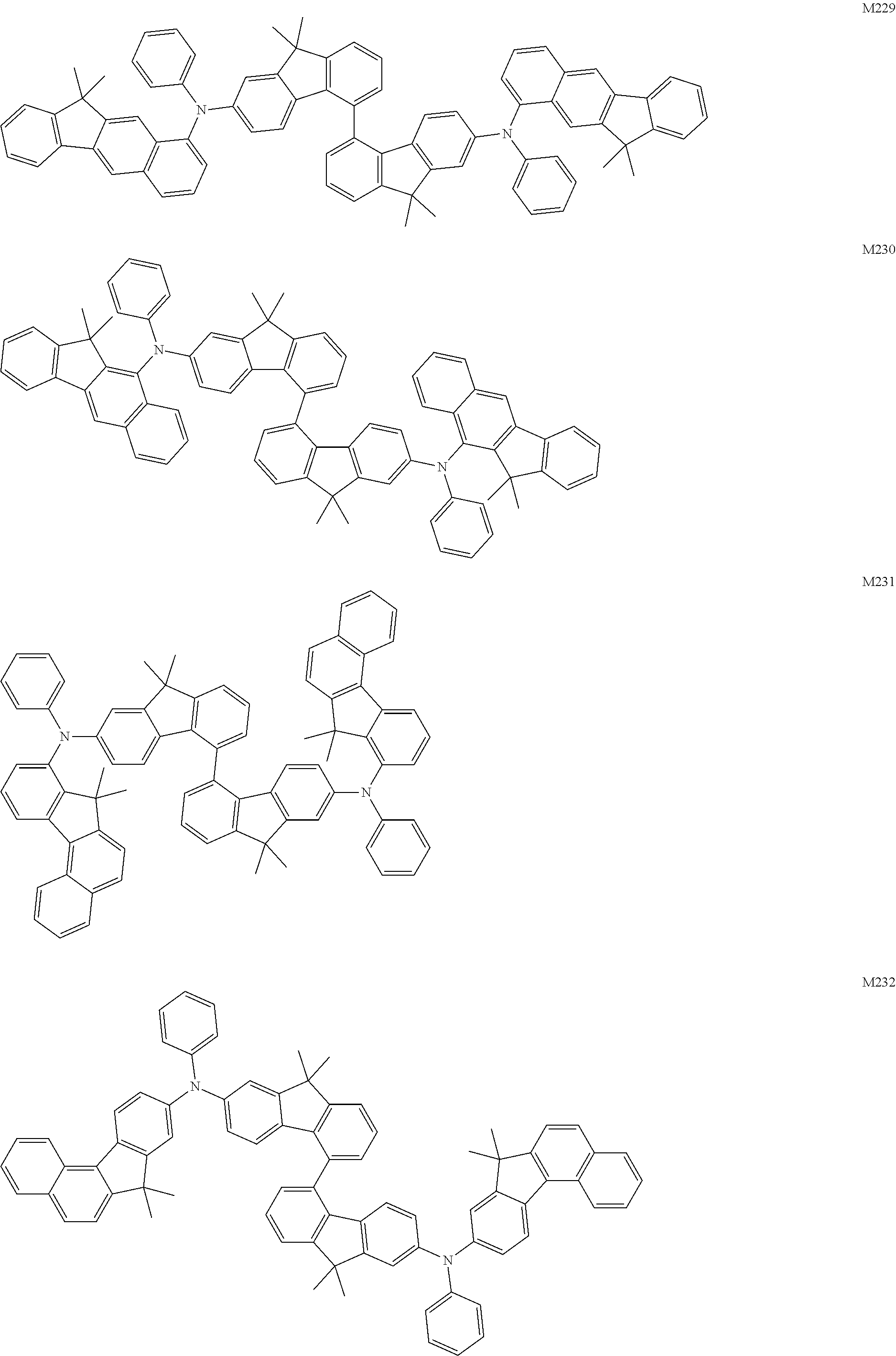

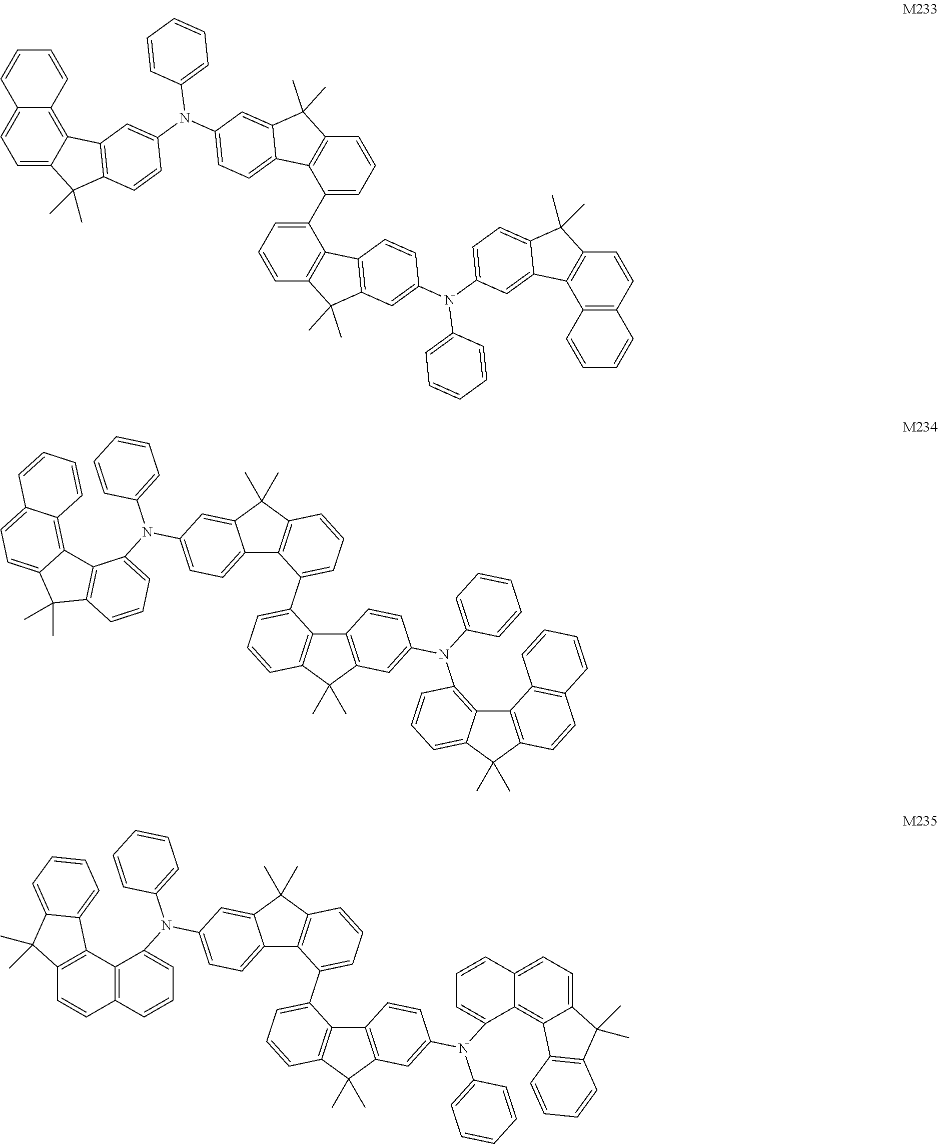

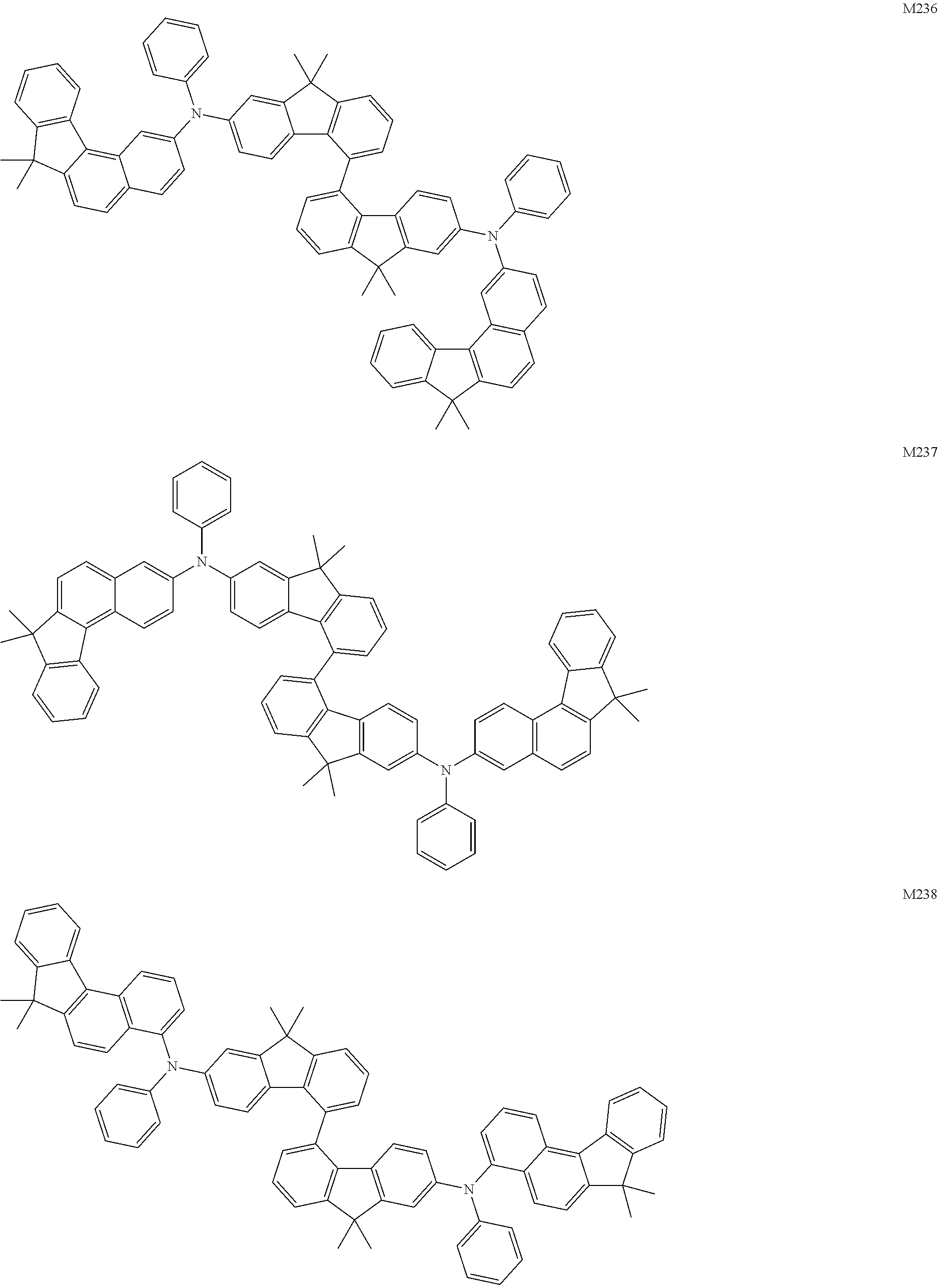

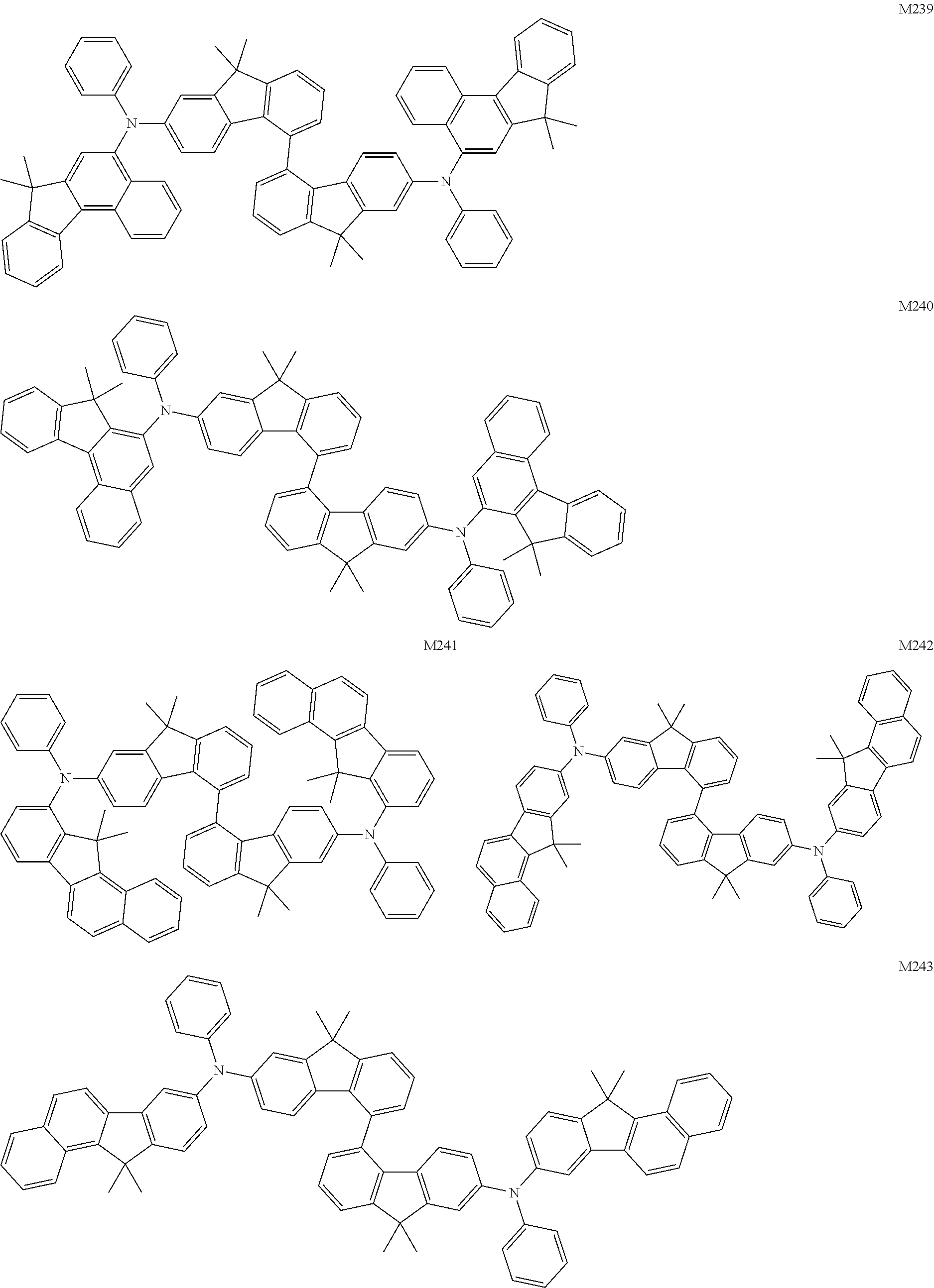

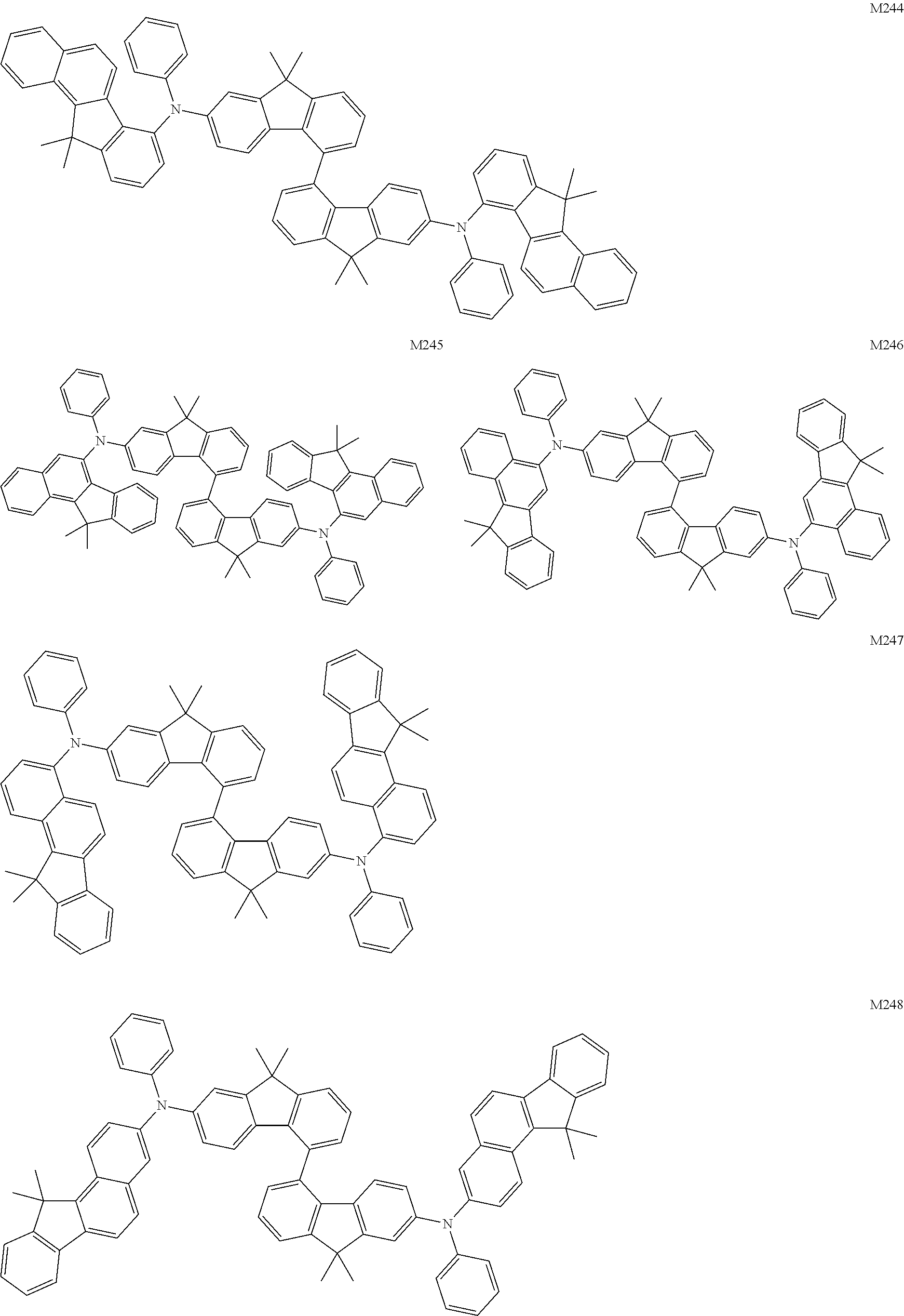

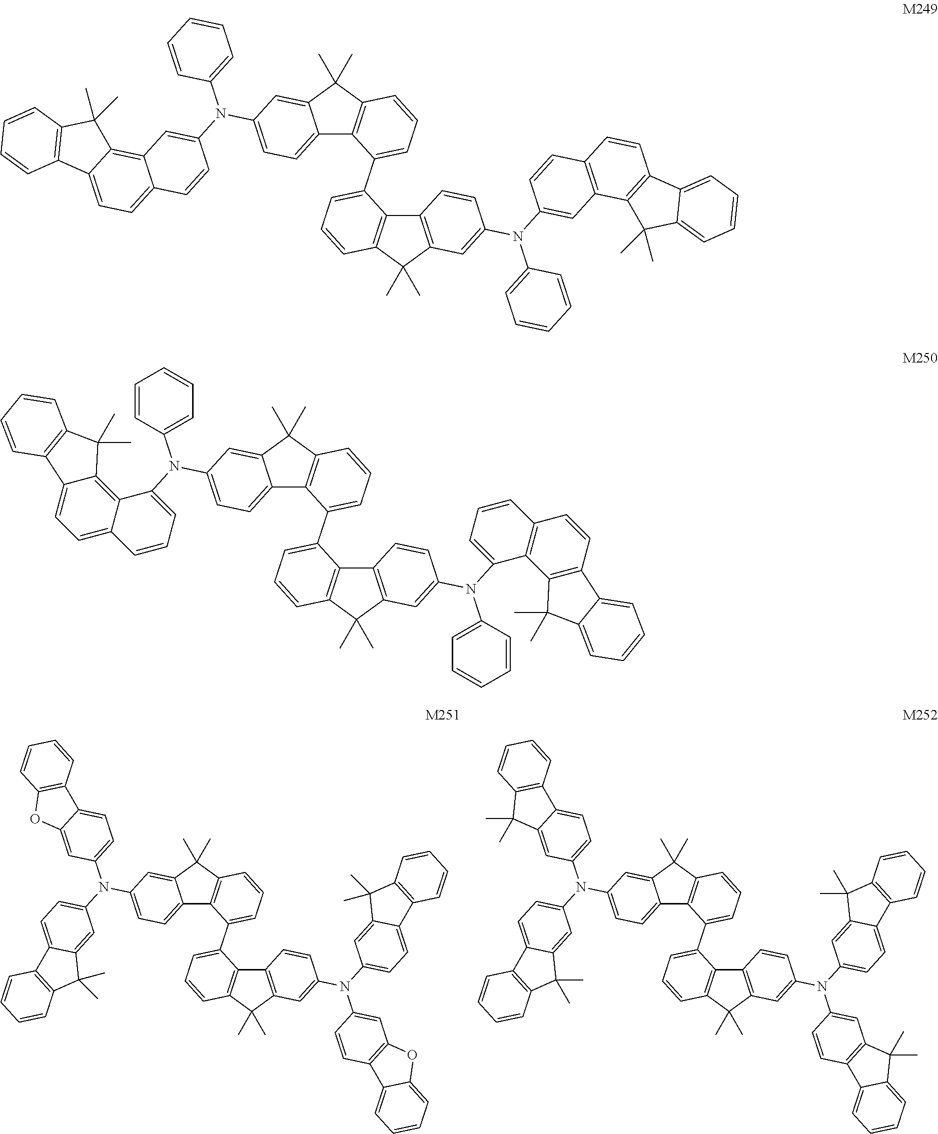

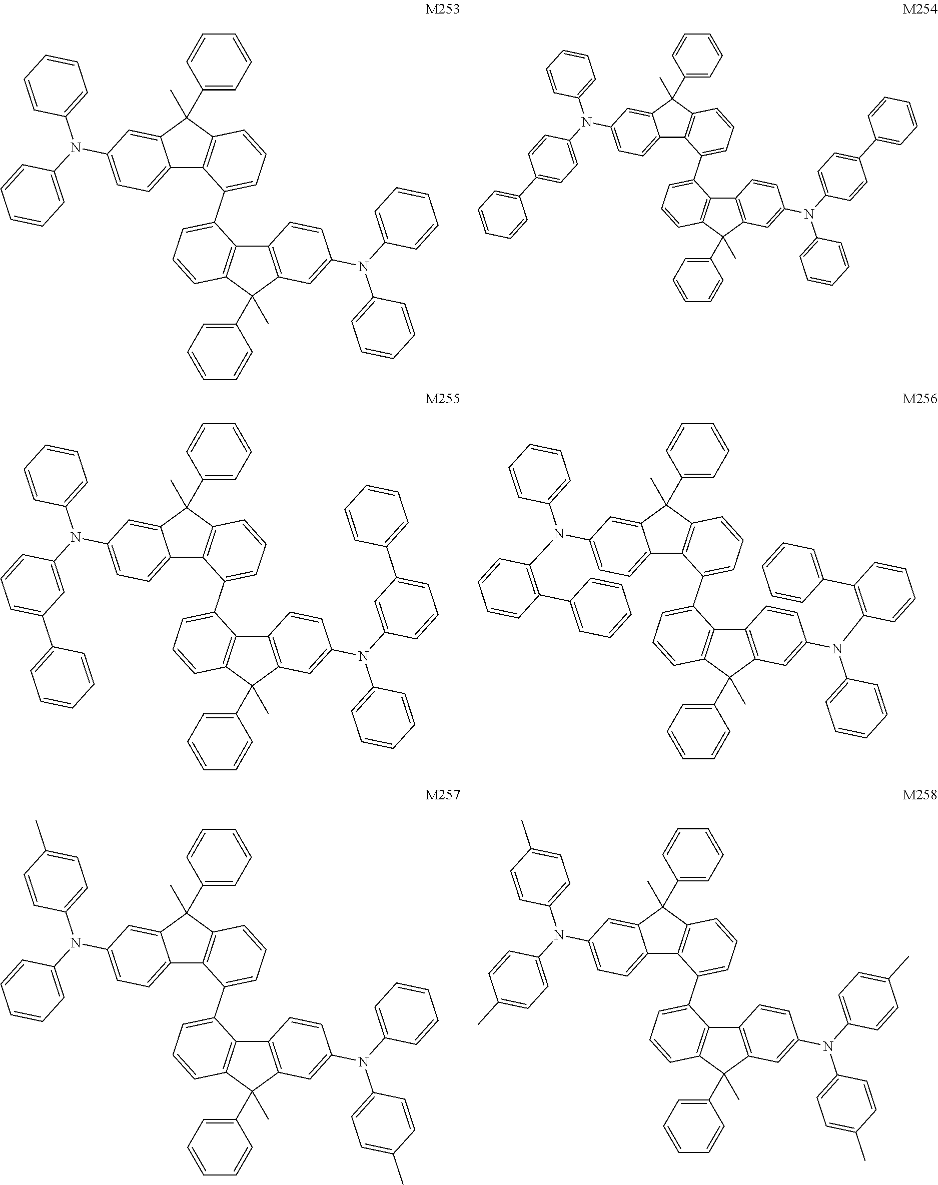

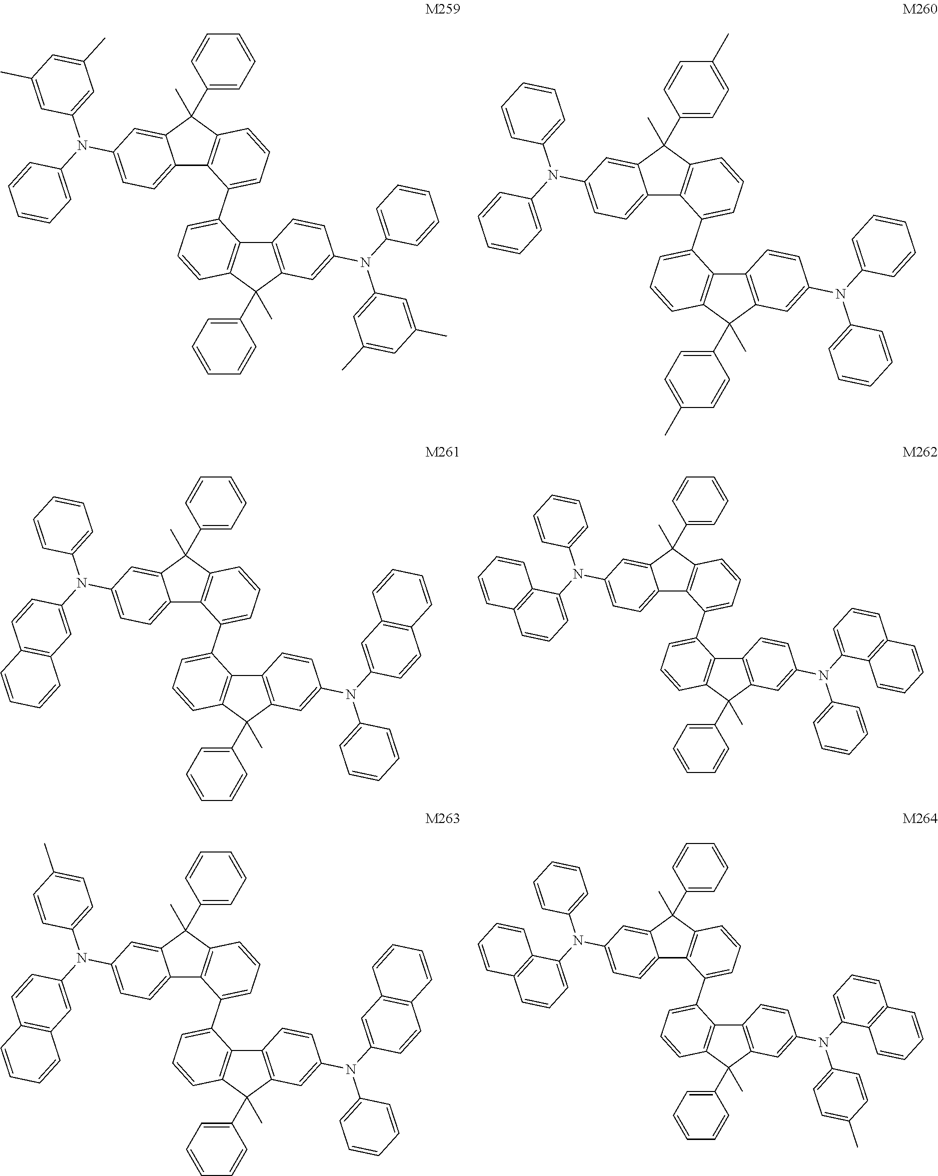

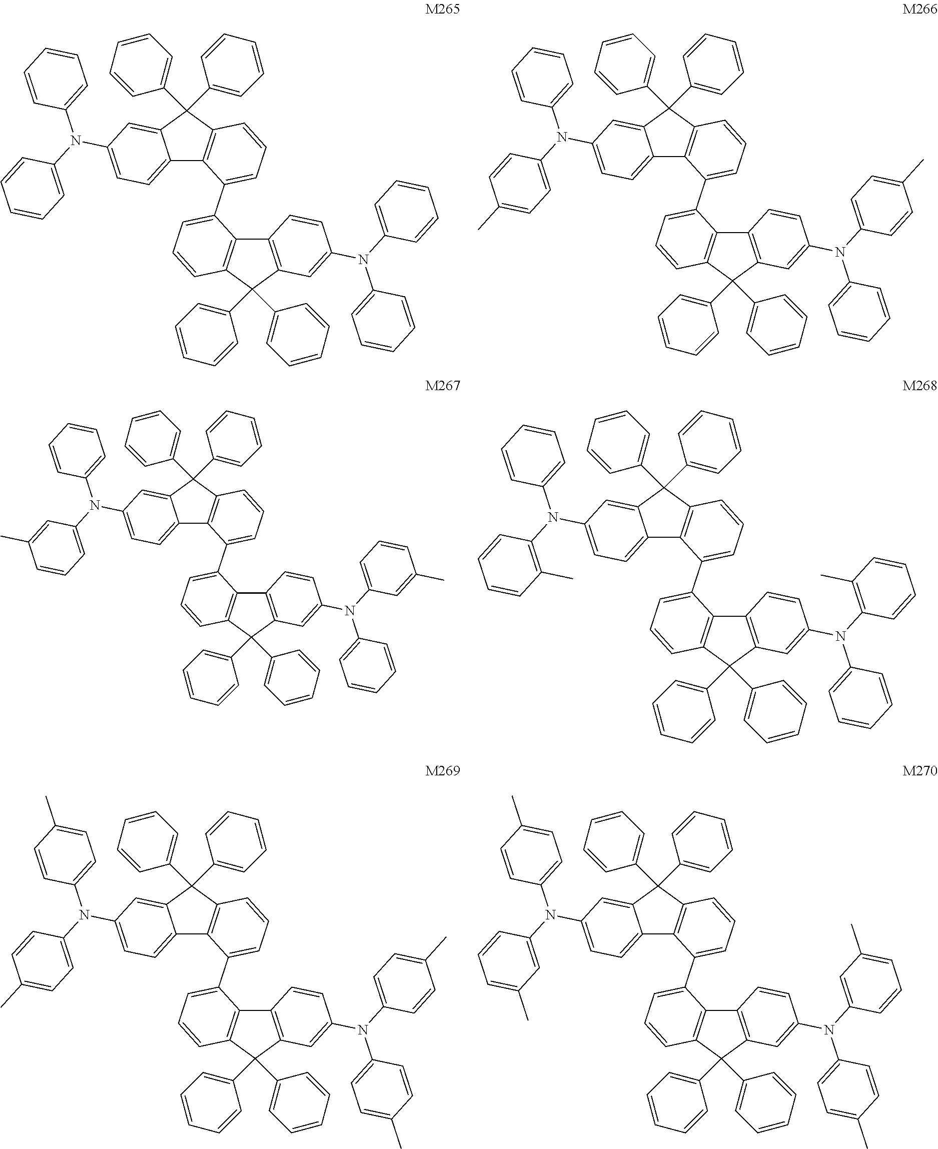

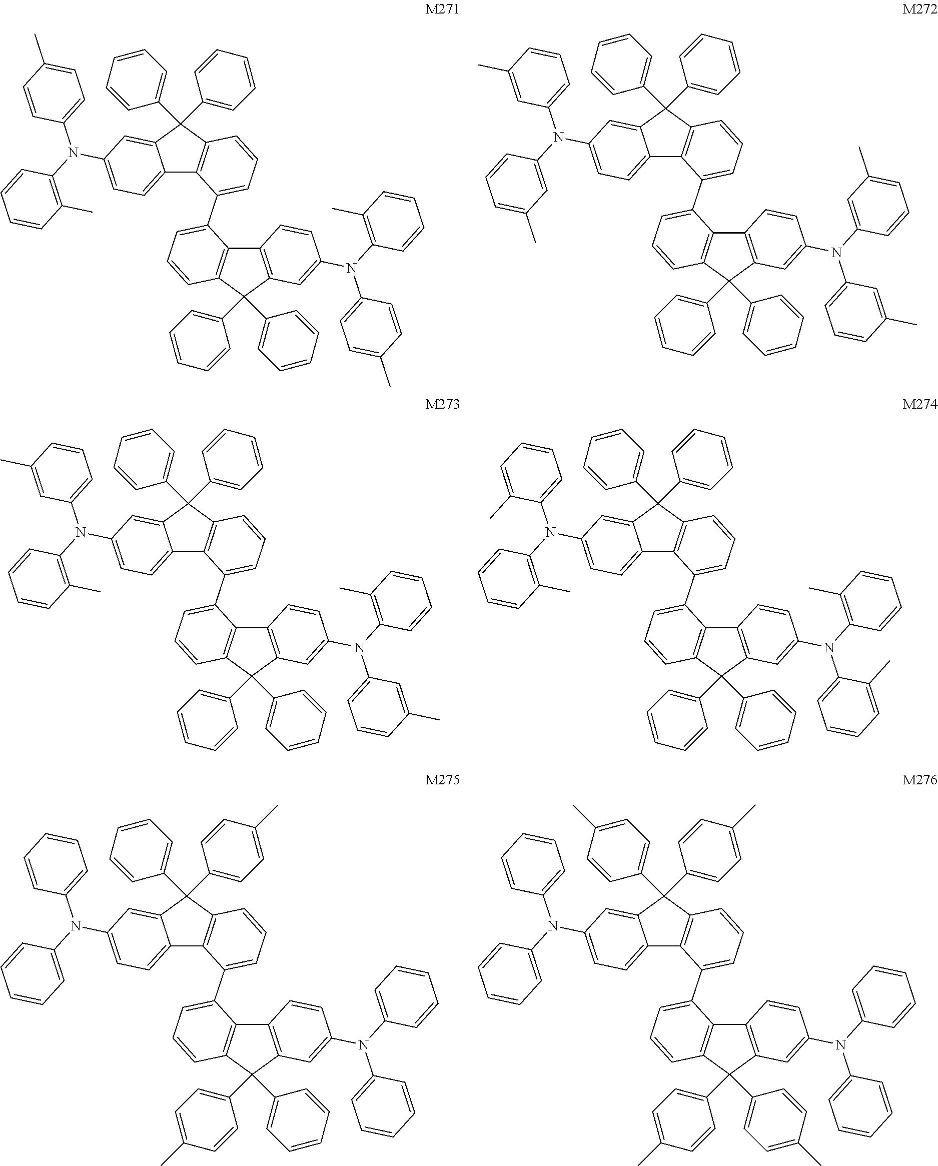

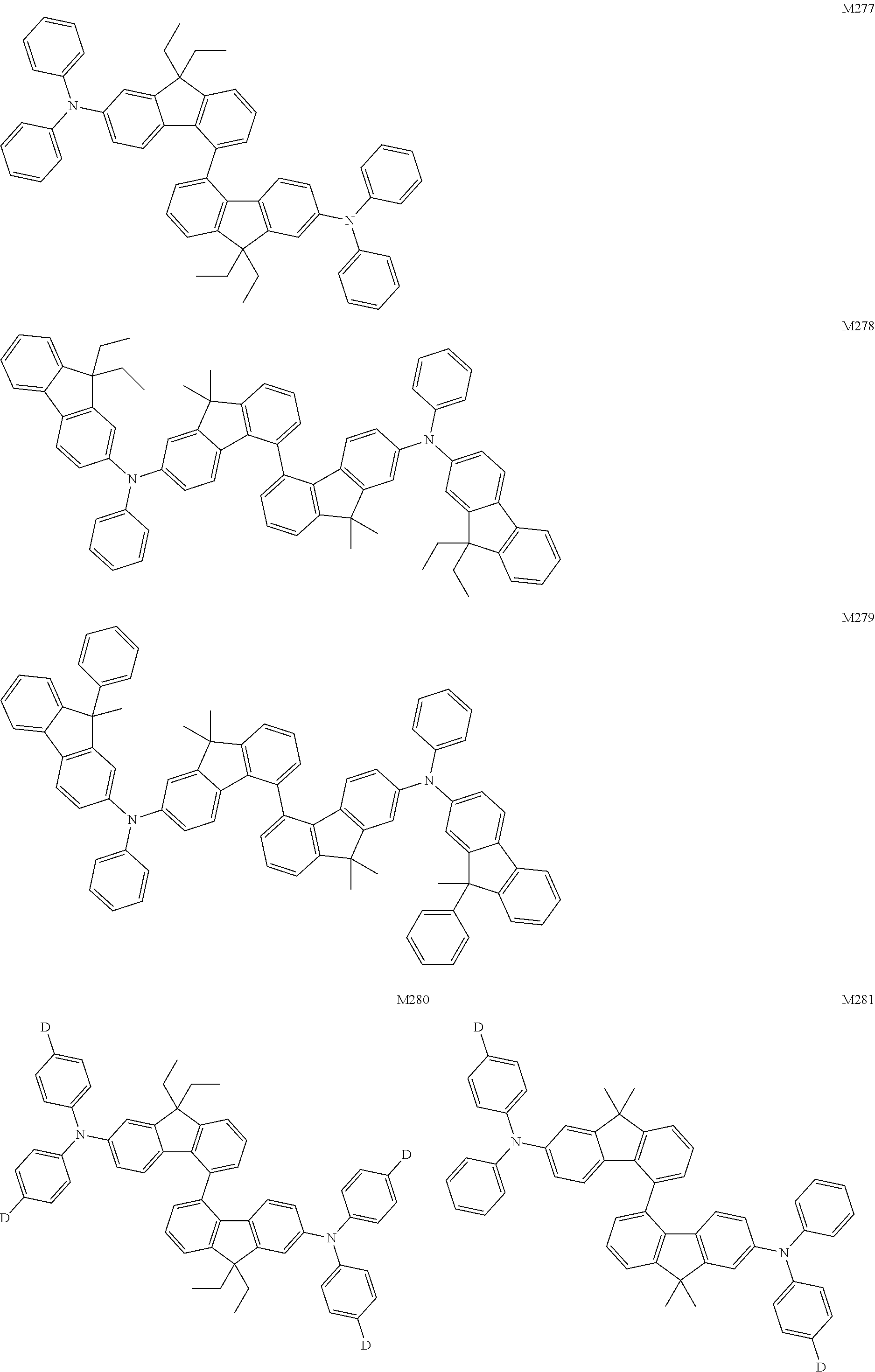

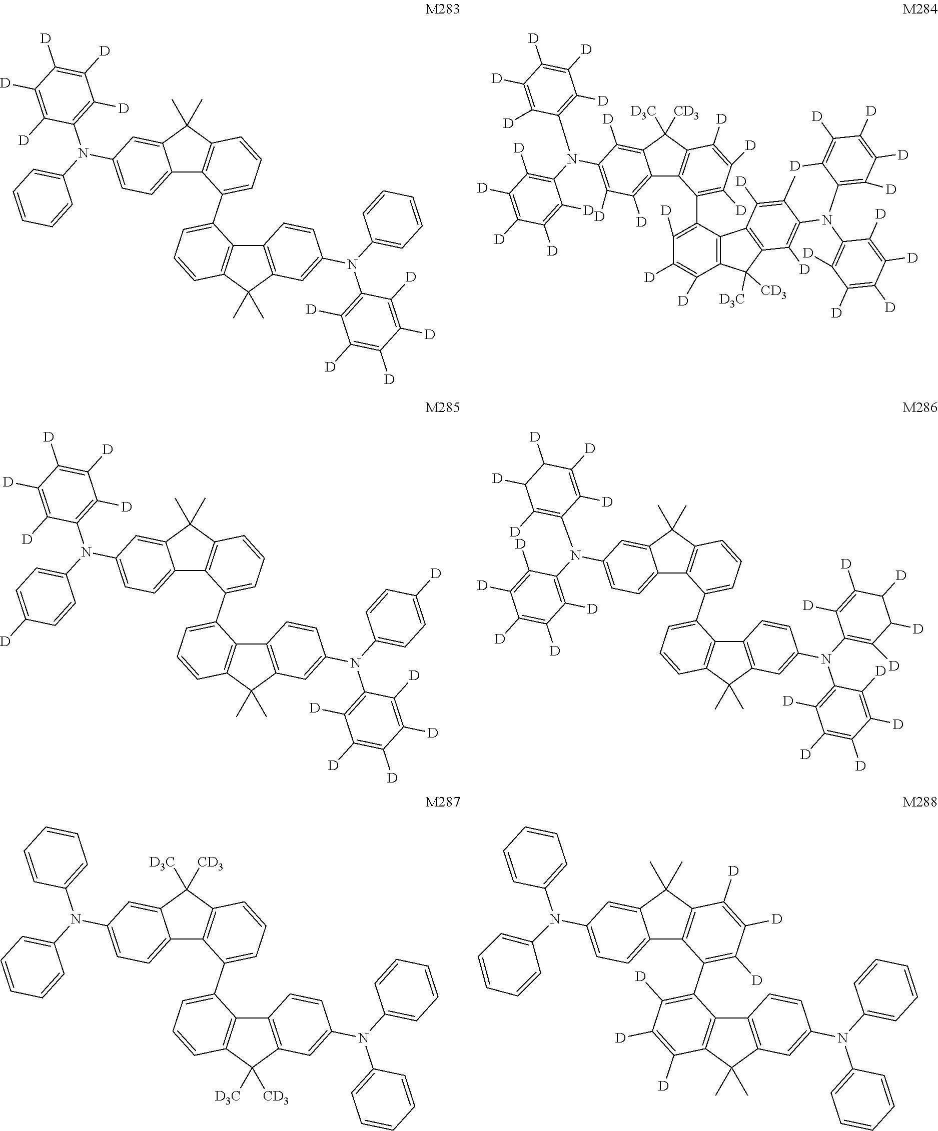

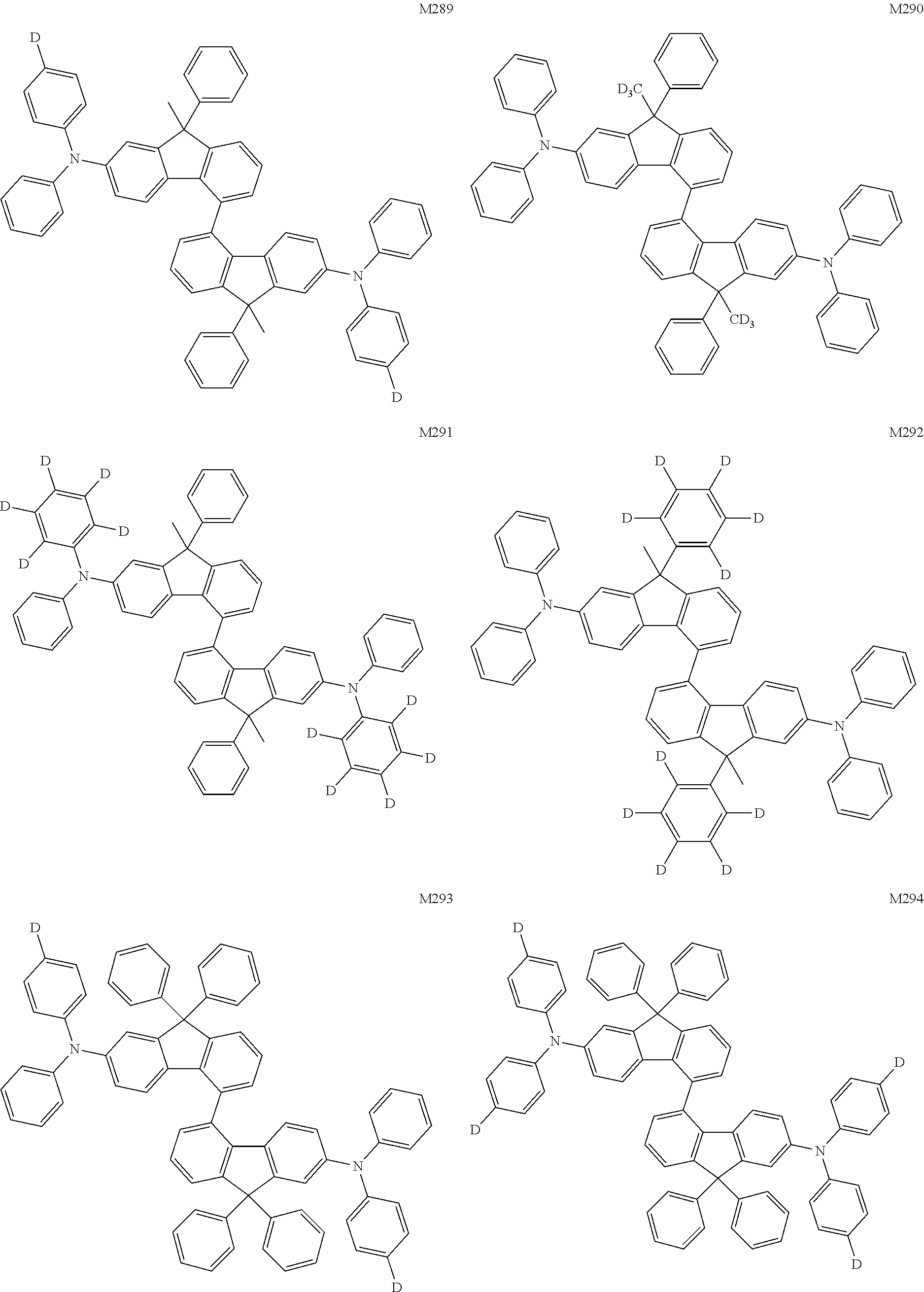

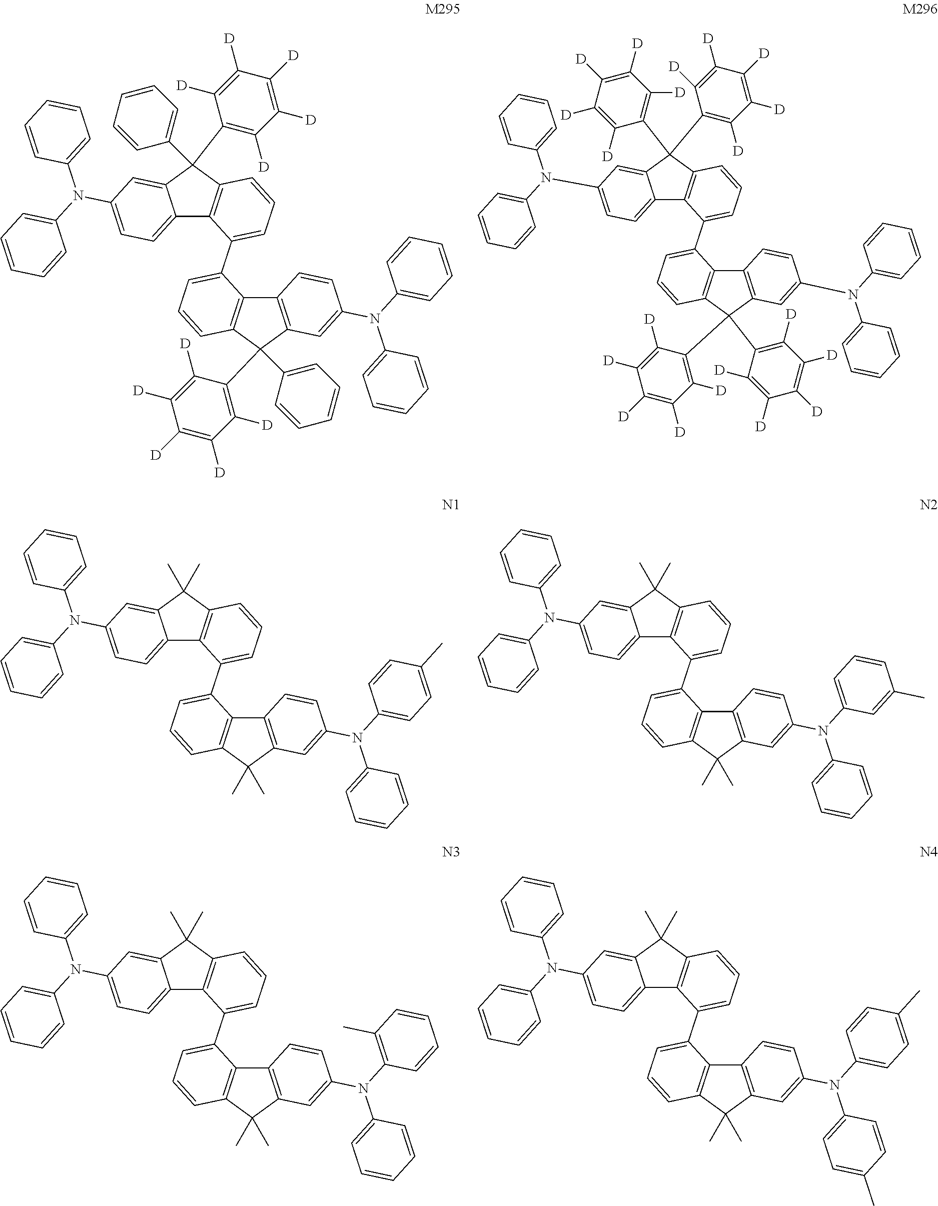

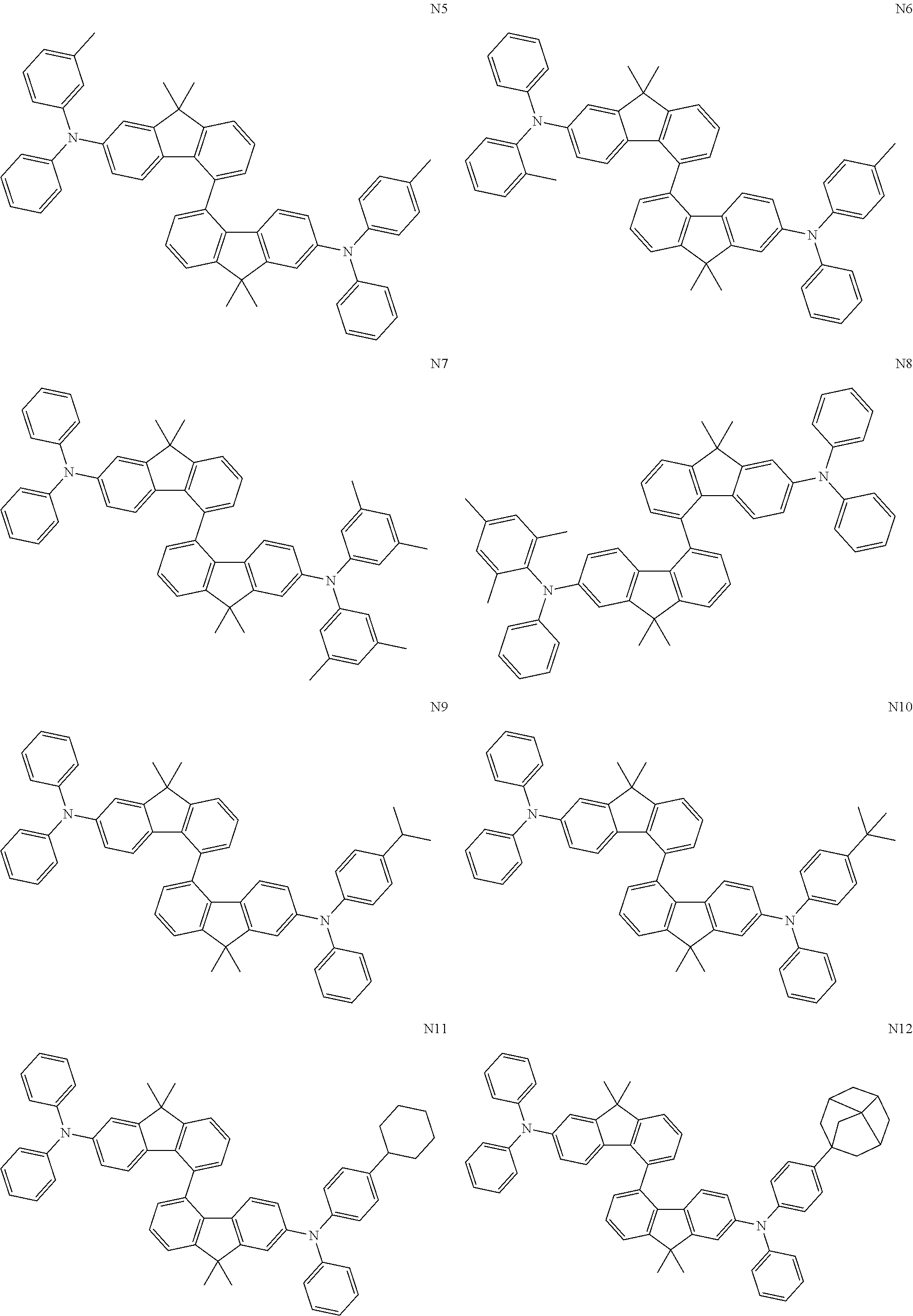

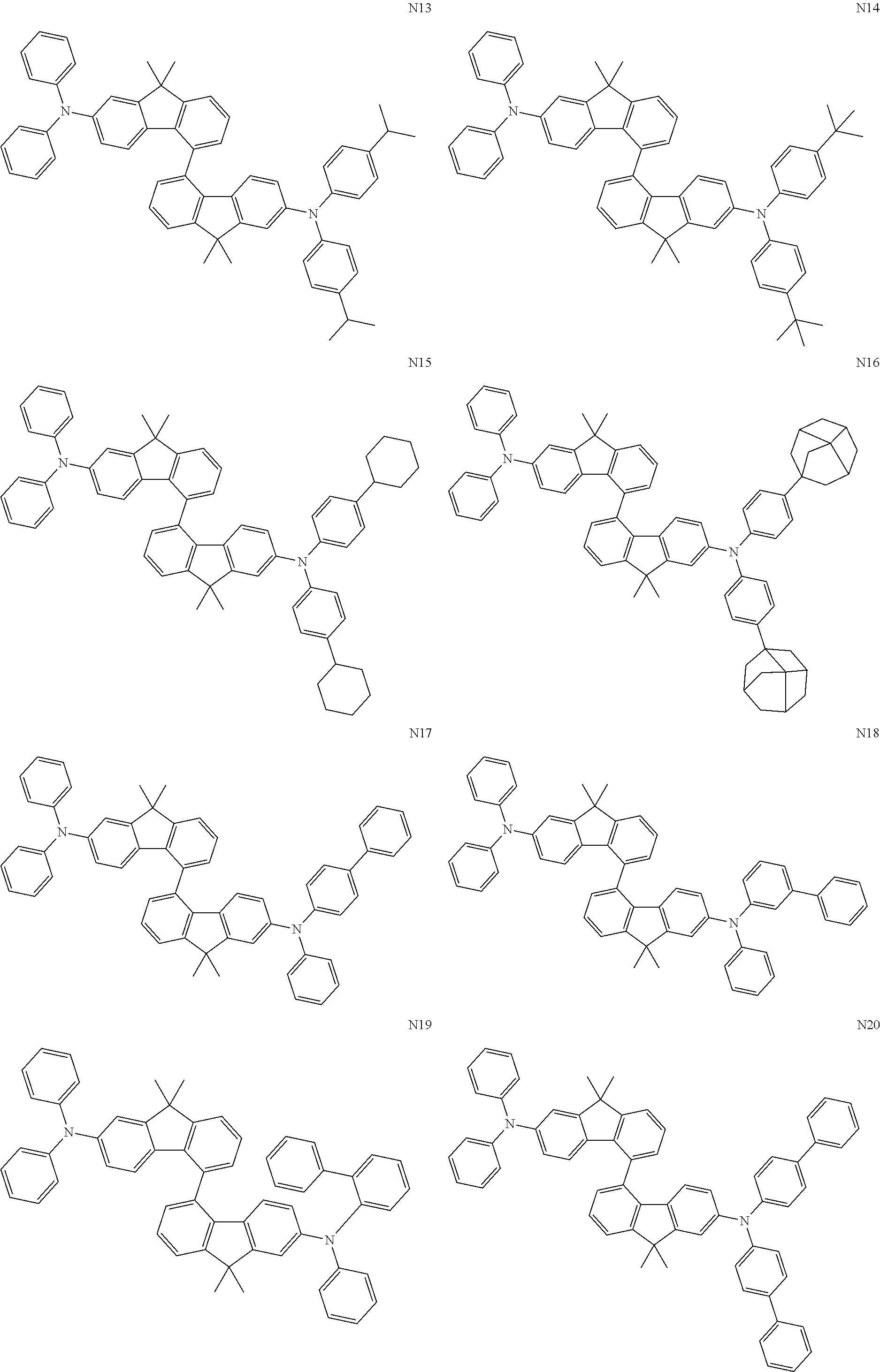

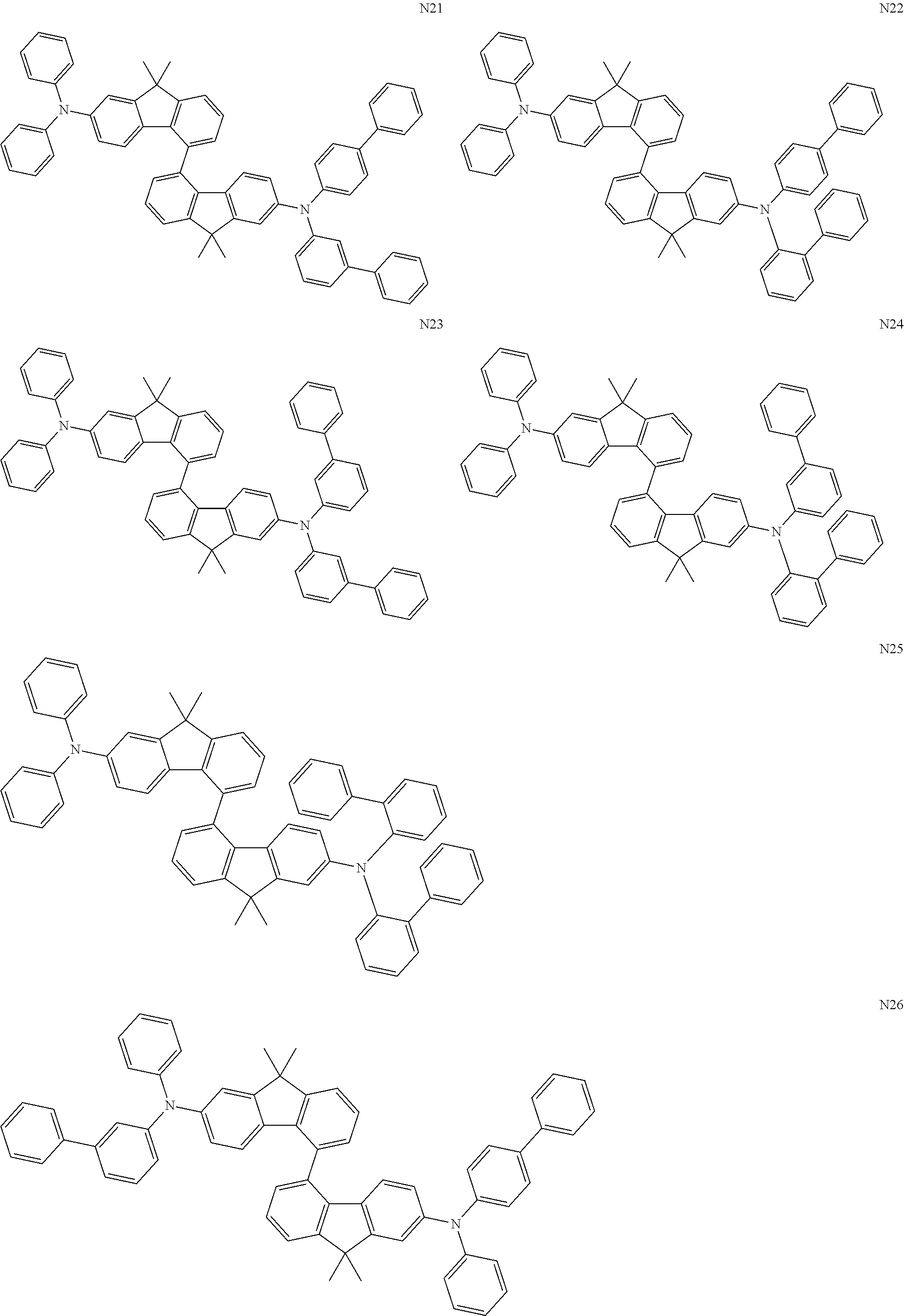

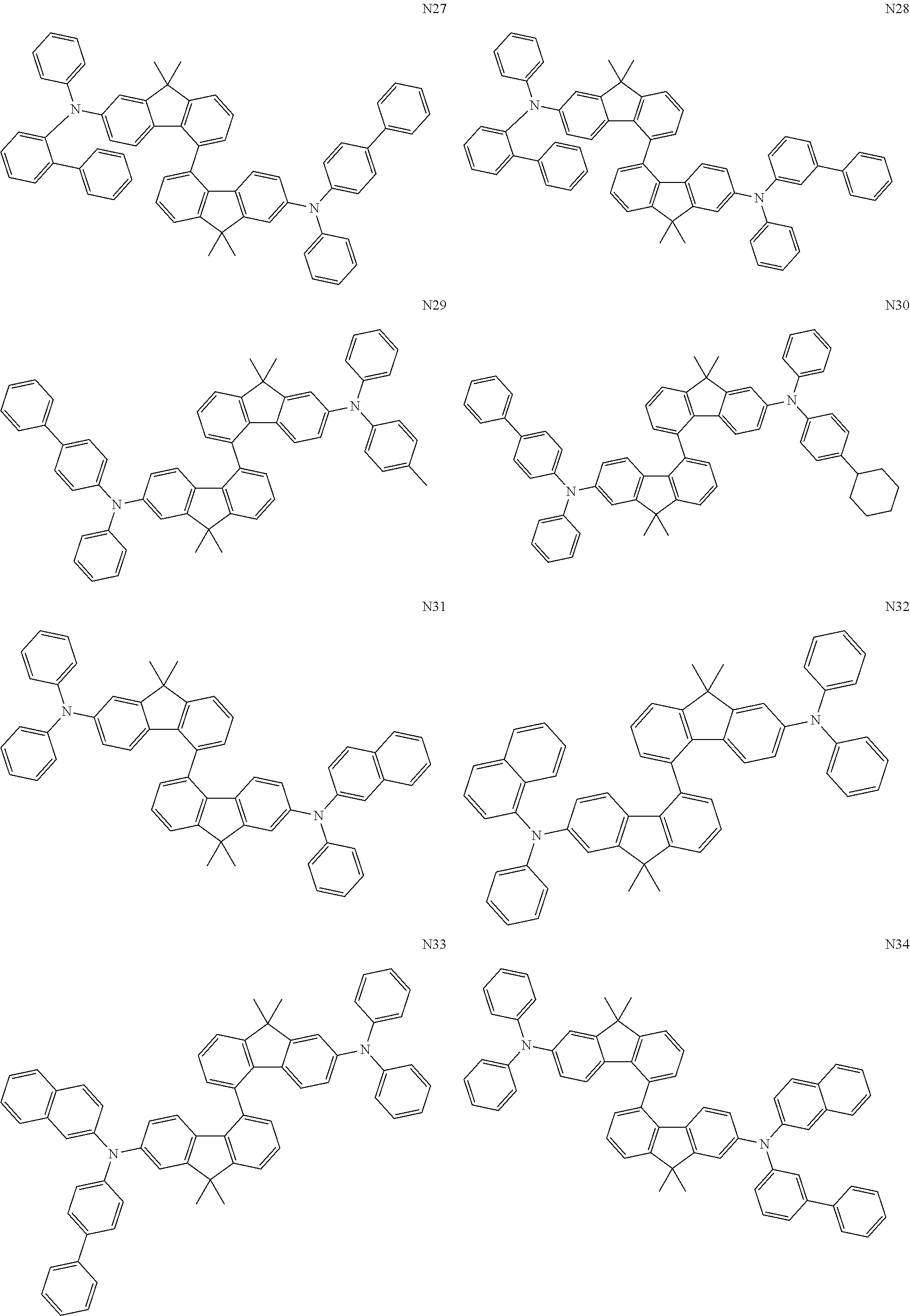

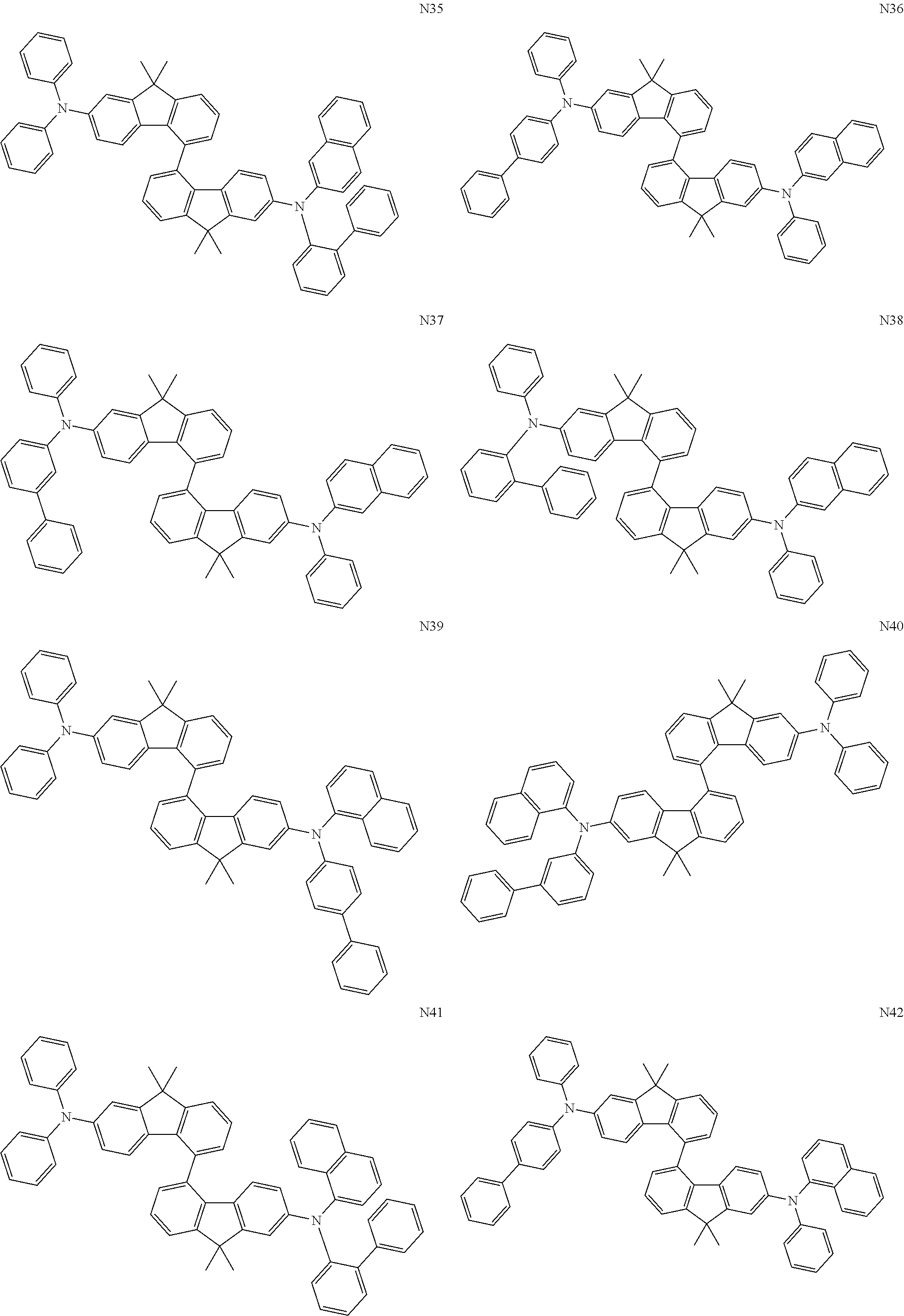

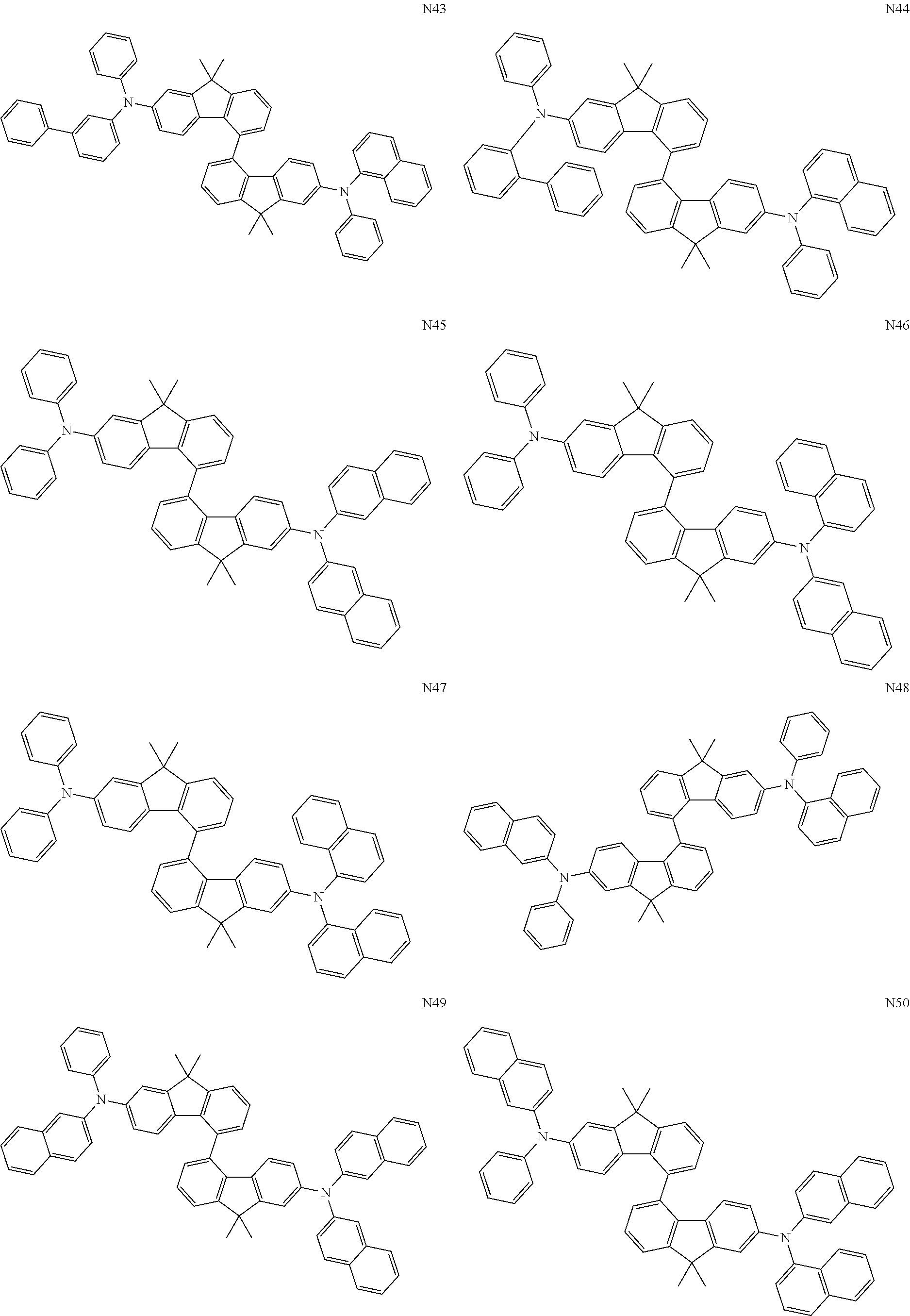

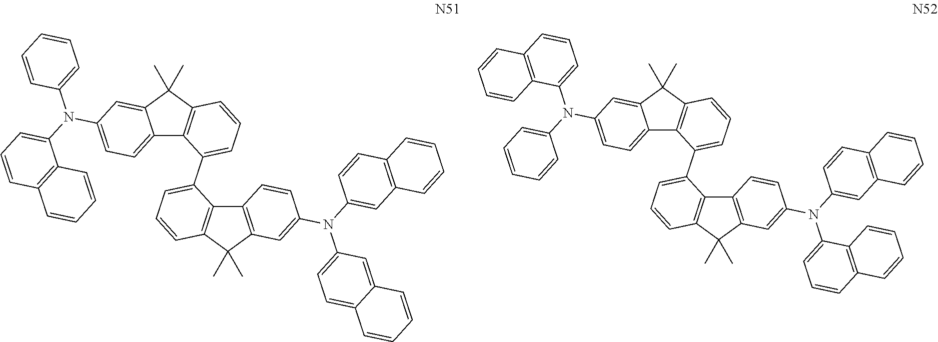 [SMILES: CC1(C)c2cc(N(c3ccc4ccccc4c3)c3ccc4ccccc4c3)ccc2-c2c(-c3cccc4c3-c3ccc(N(c5ccccc5)c5cccc6ccccc56)cc3C4(C)C)cccc21 ; CC1(C)c2cc(N(c3ccccc3)c3cccc4ccccc34)ccc2-c2c(-c3cccc4c3-c3ccc(N(c5ccc6ccccc6c5)c5cccc6ccccc56)cc3C4(C)C)cccc21]

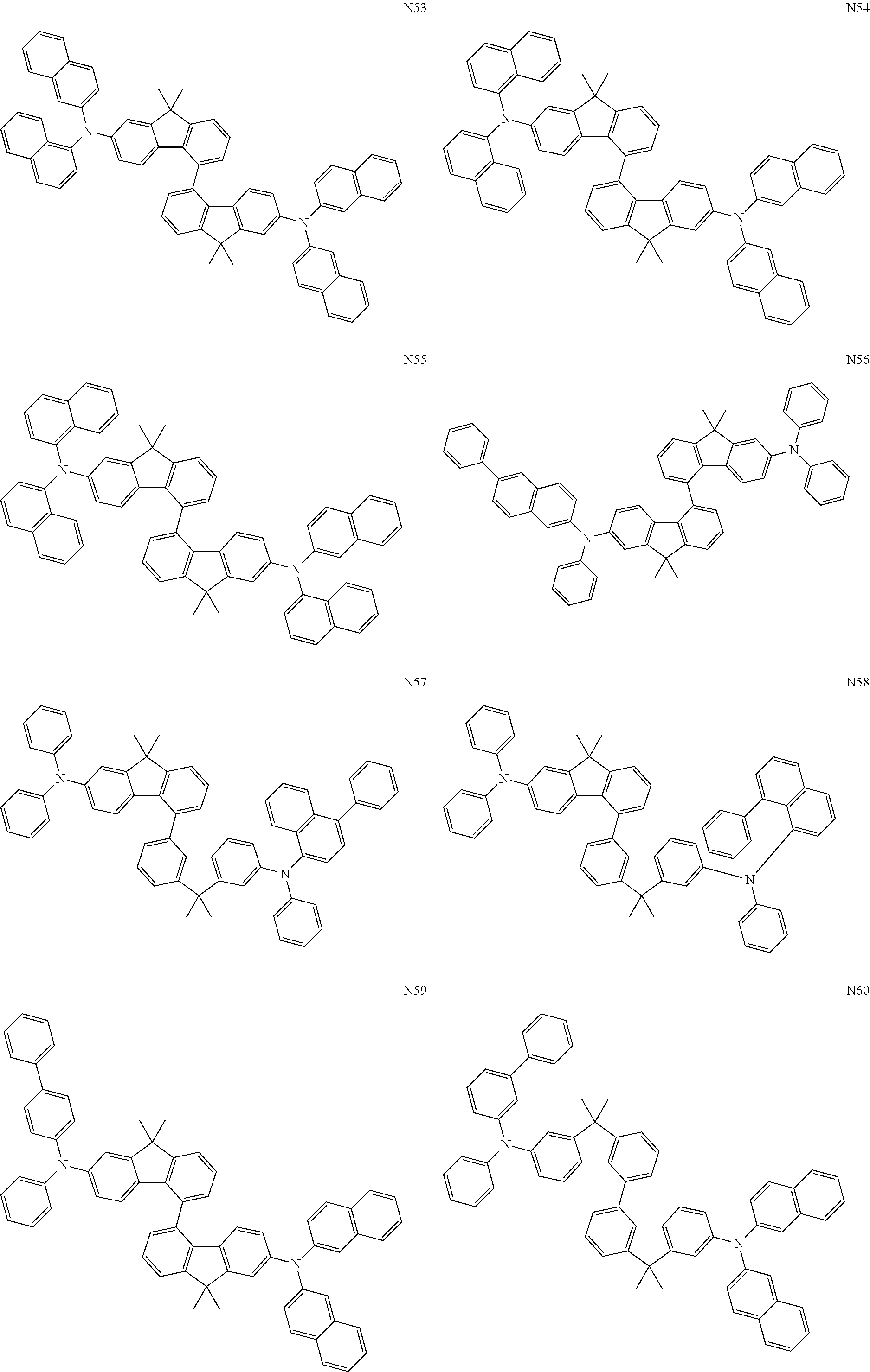

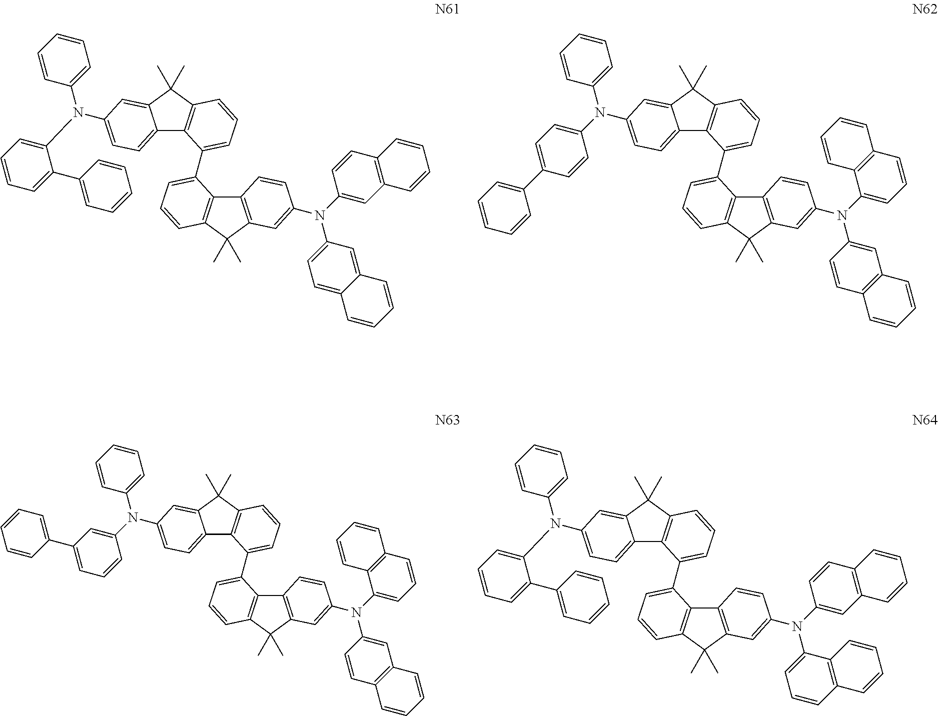 [SMILES: CC1(C)c2cc(N(c3ccc4ccccc4c3)c3ccc4ccccc4c3)ccc2-c2c(-c3cccc4c3-c3ccc(N(c5ccccc5)c5ccccc5-c5ccccc5)cc3C4(C)C)cccc21 ; CC1(C)c2cc(N(c3ccccc3)c3ccc(-c4ccccc4)cc3)ccc2-c2c(-c3cccc4c3-c3ccc(N(c5ccc6ccccc6c5)c5cccc6ccccc56)cc3C4(C)C)cccc21 ; CC1(C)c2cc(N(c3ccccc3)c3cccc(-c4ccccc4)c3)ccc2-c2c(-c3cccc4c3-c3ccc(N(c5ccc6ccccc6c5)c5cccc6ccccc56)cc3C4(C)C)cccc21 ; CC1(C)c2cc(N(c3ccccc3)c3ccccc3-c3ccccc3)ccc2-c2c(-c3cccc4c3-c3ccc(N(c5ccc6ccccc6c5)c5cccc6ccccc56)cc3C4(C)C)cccc21]

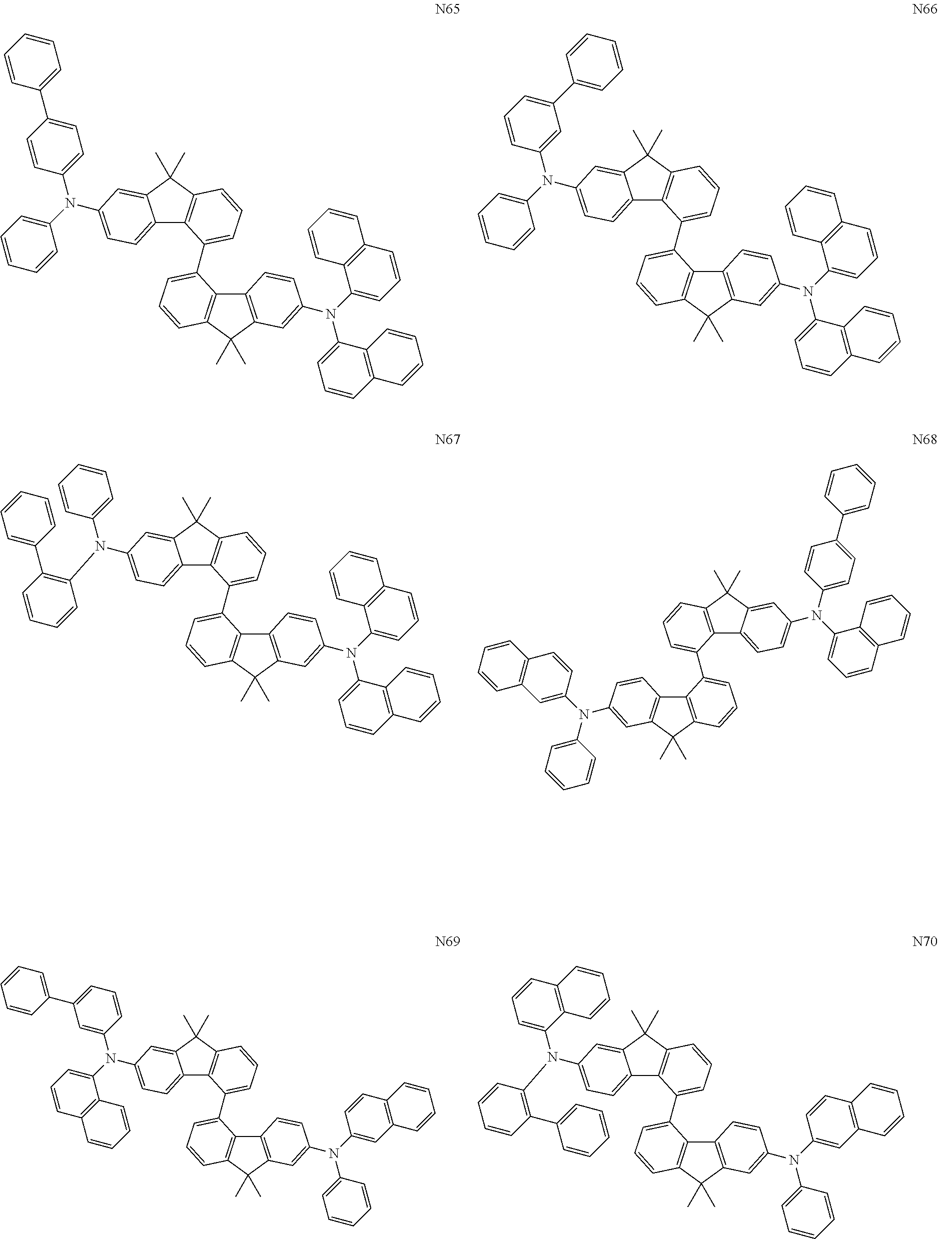

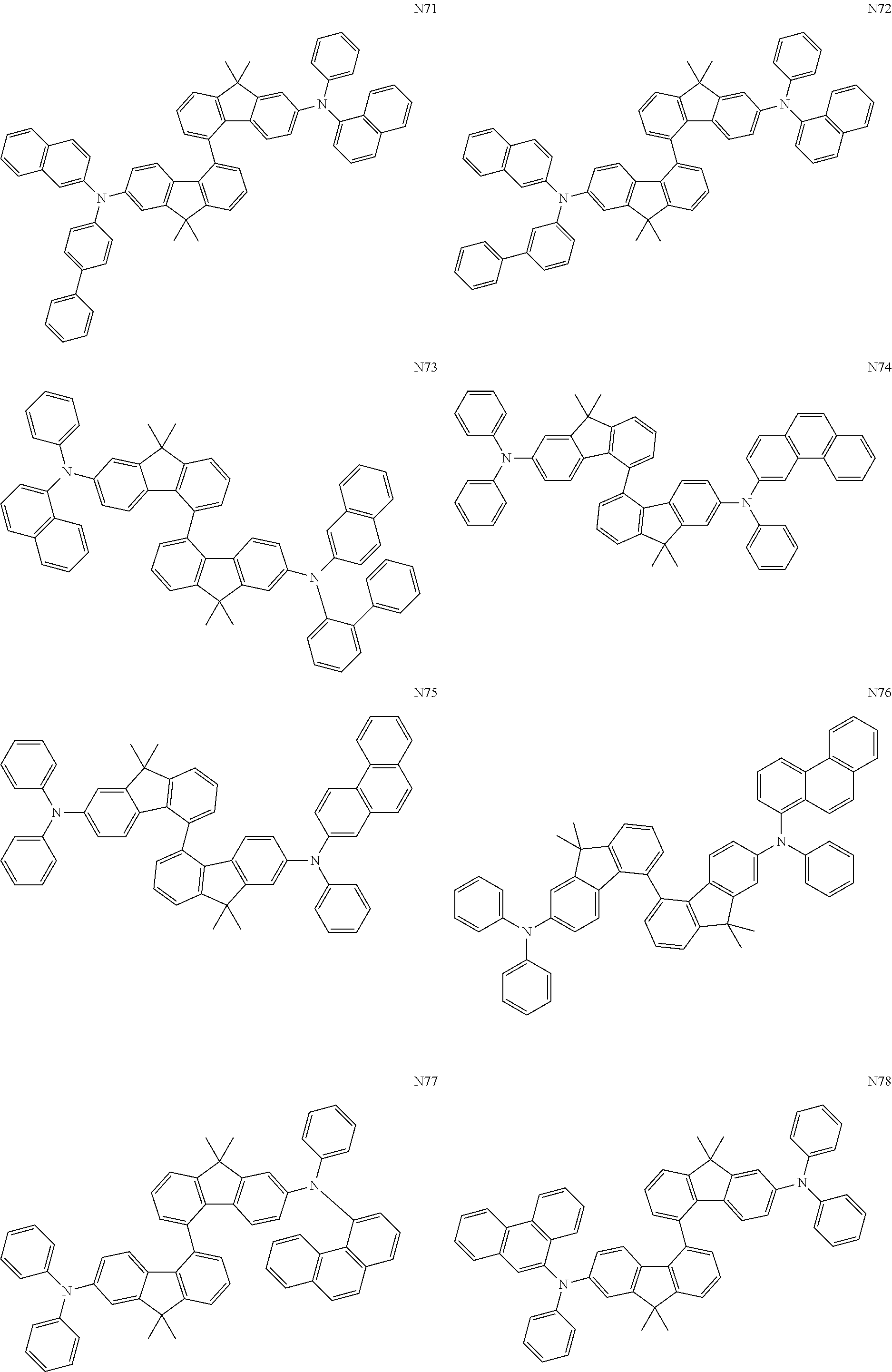

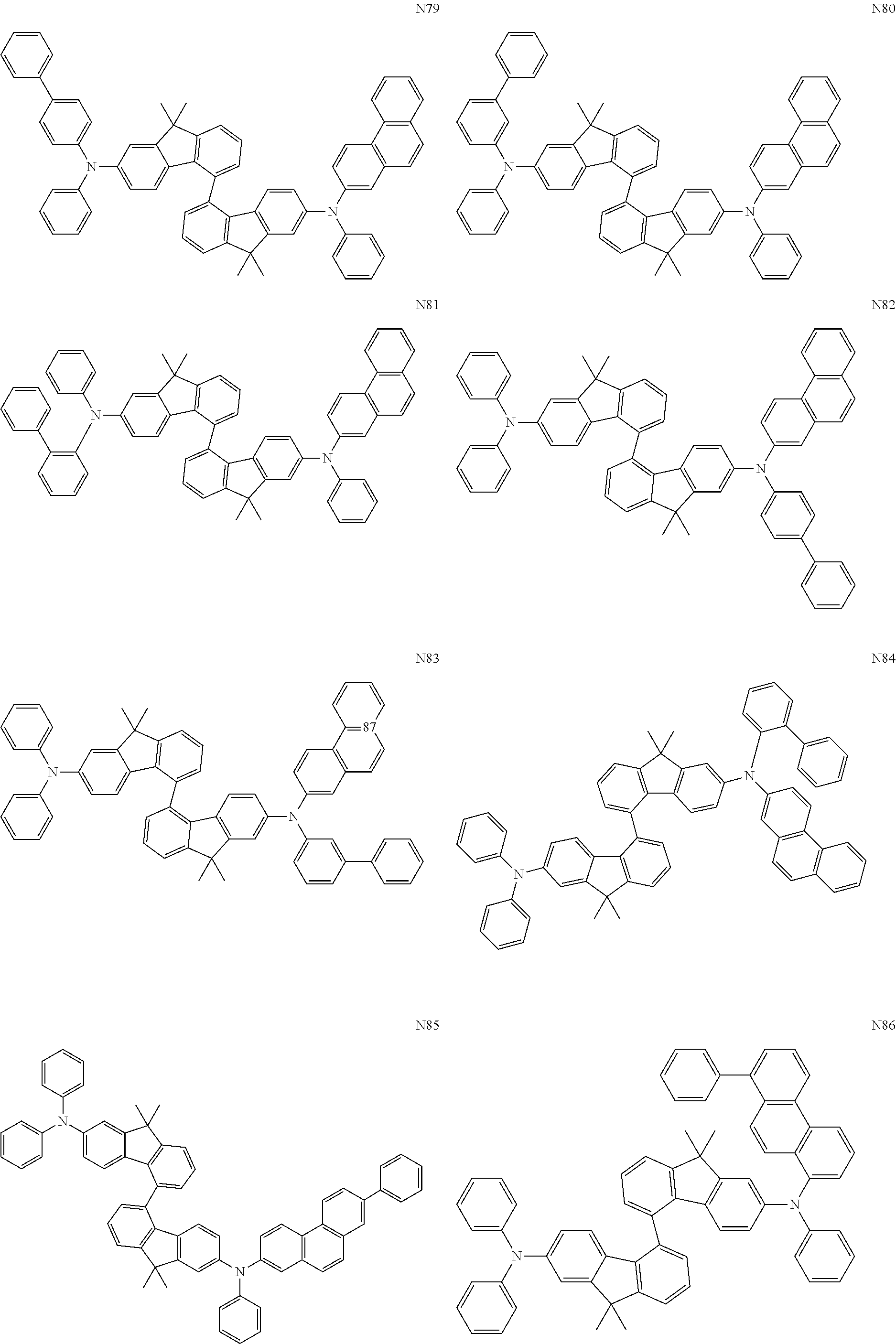

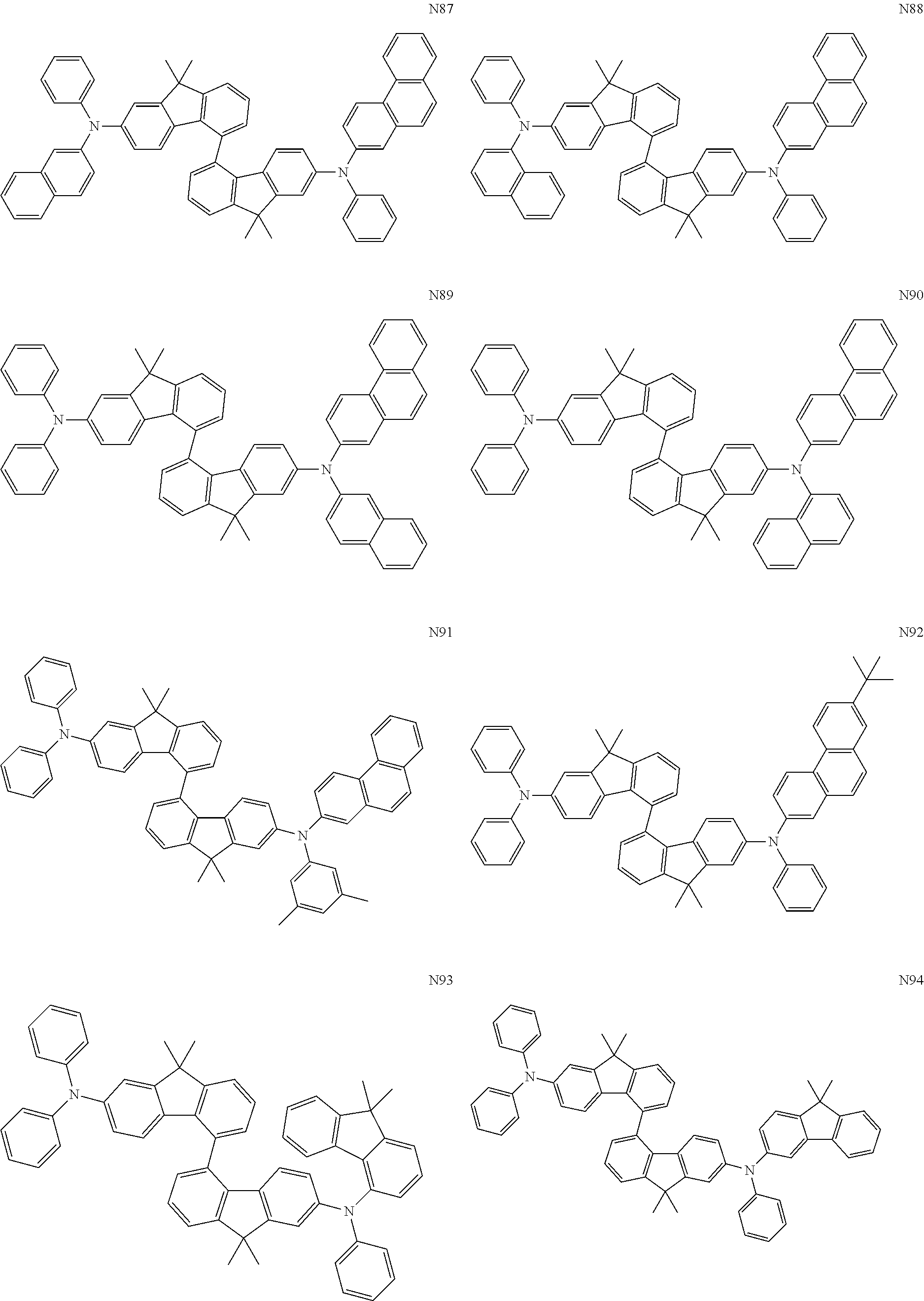

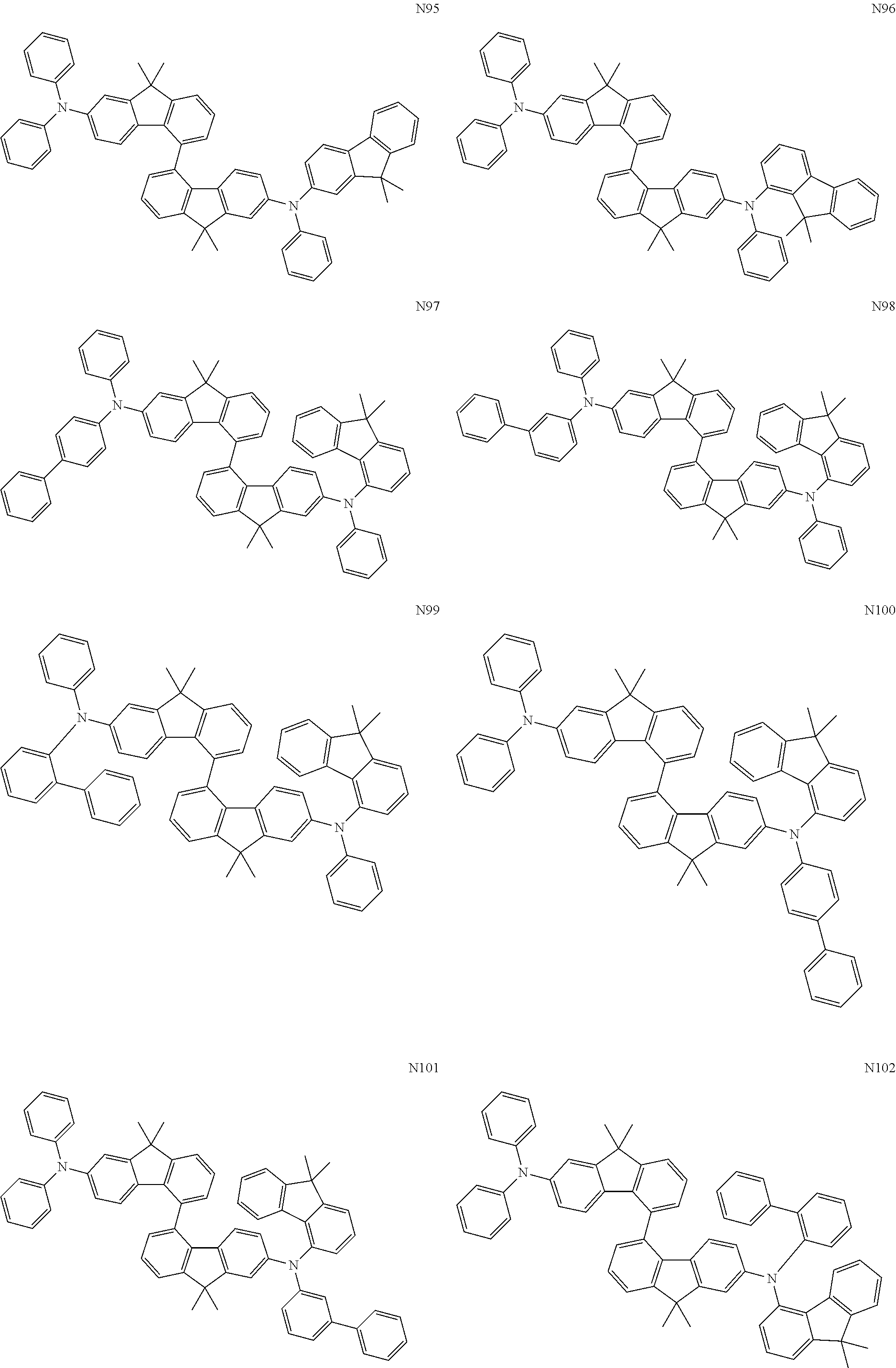

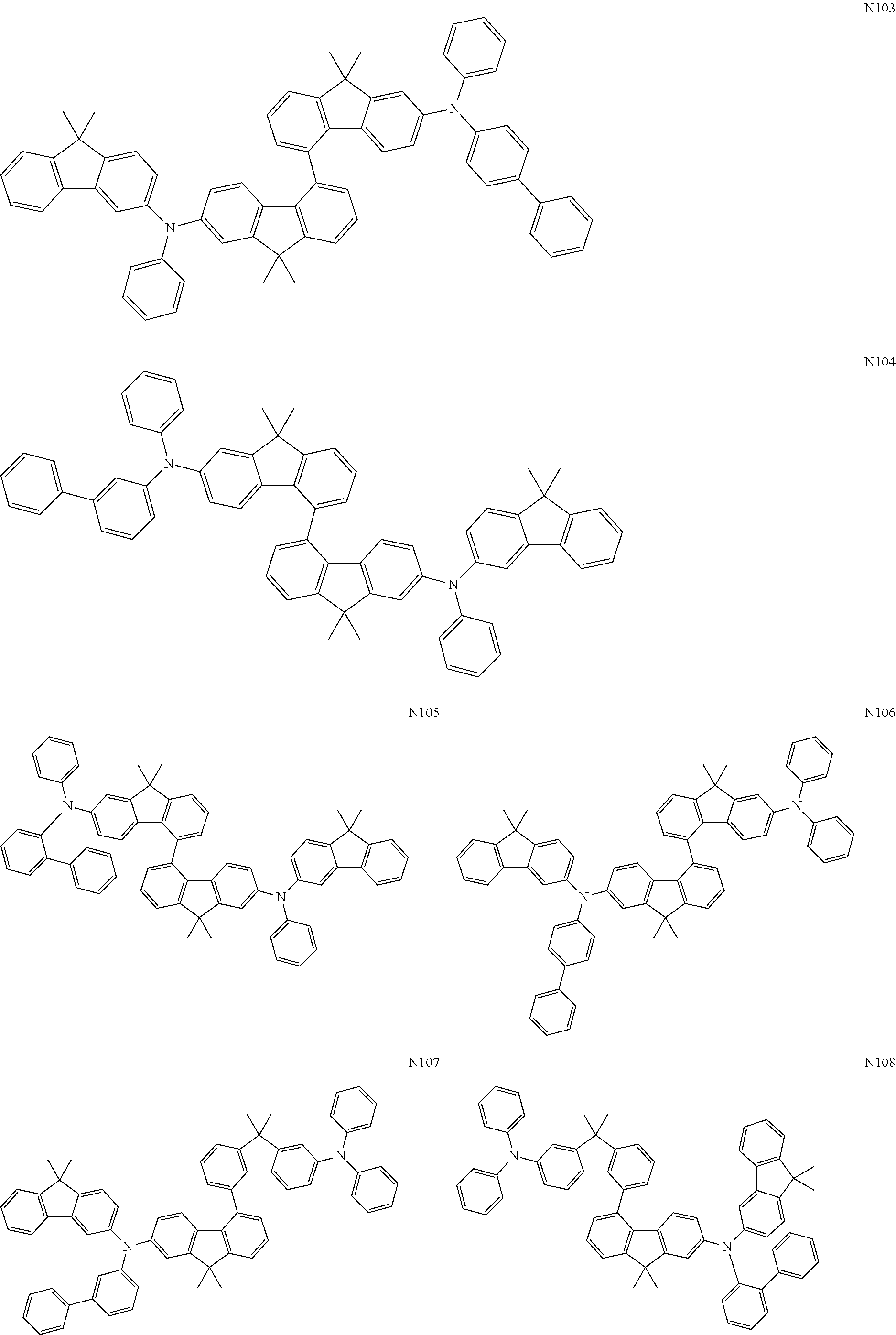

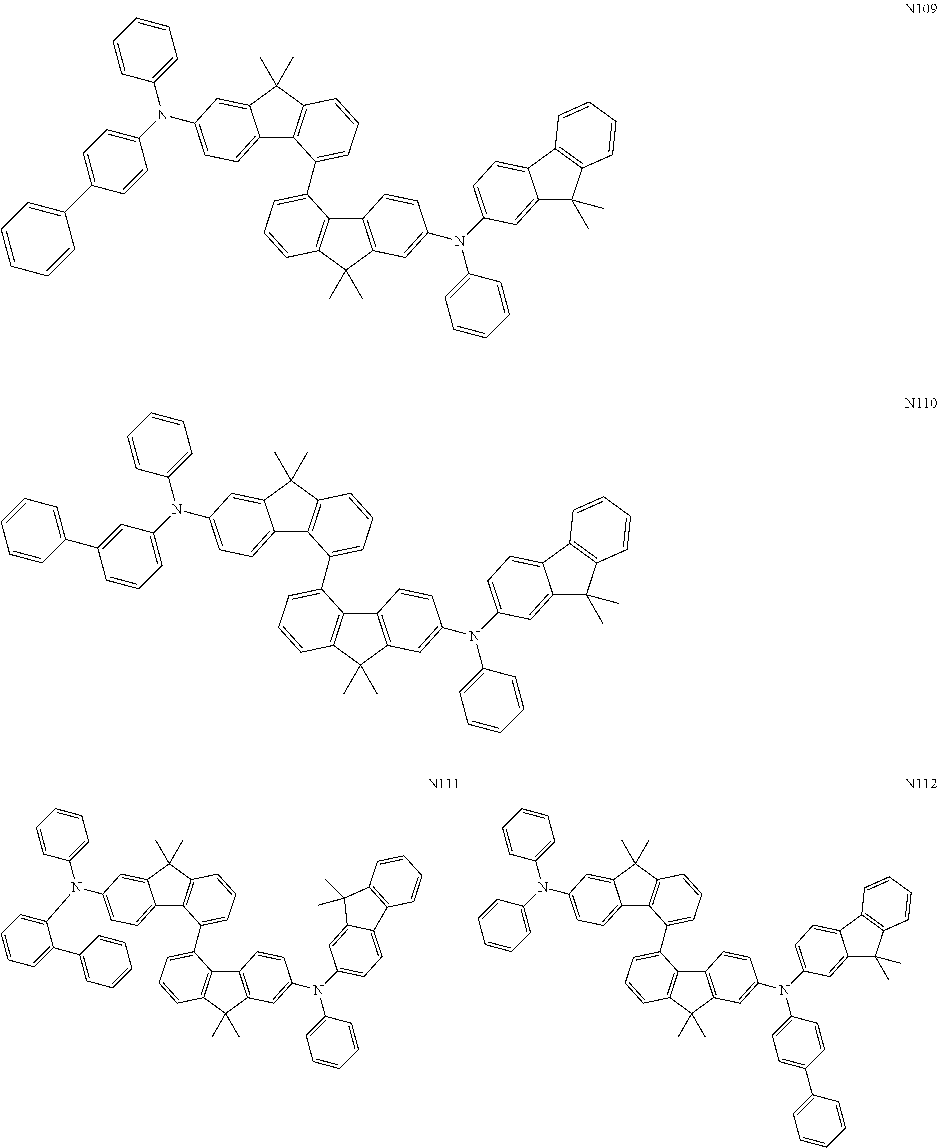

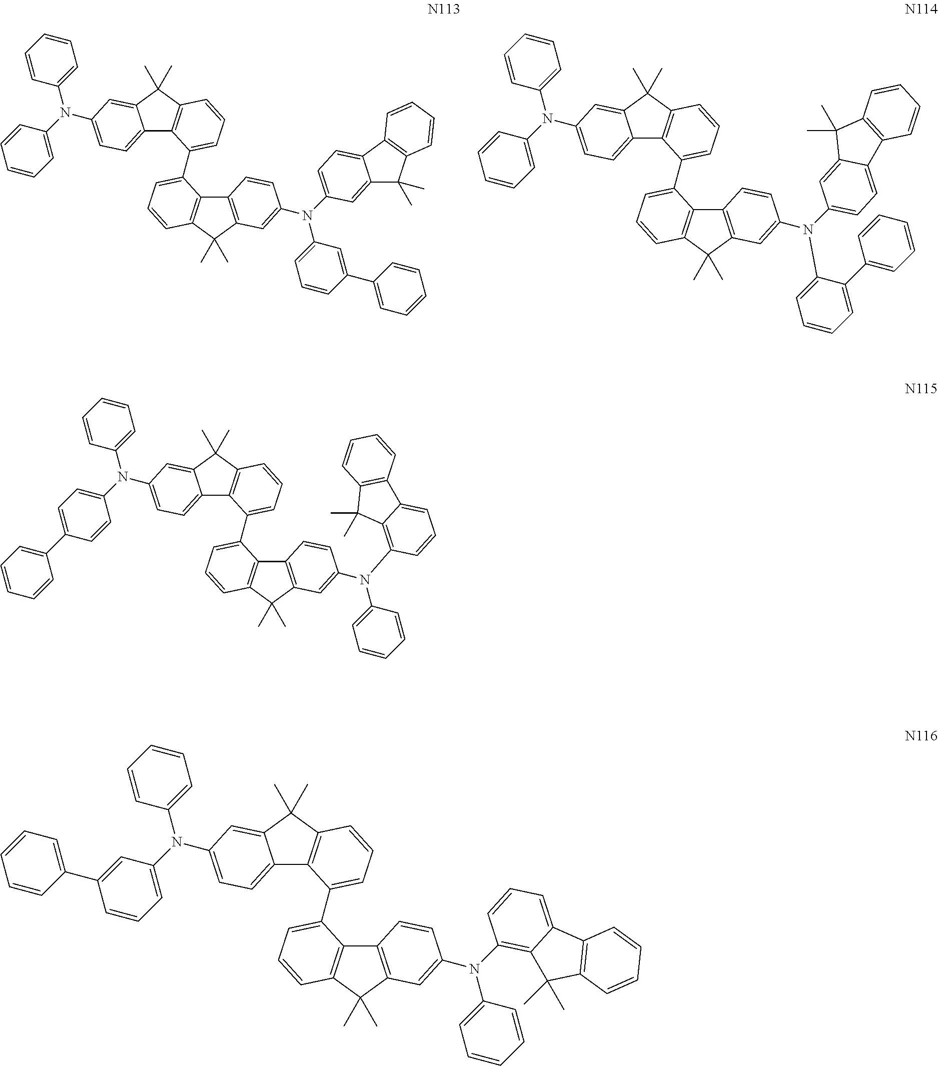 [SMILES: CC1(C)c2ccccc2-c2ccc(N(c3cccc(-c4ccccc4)c3)c3ccc4c(c3)C(C)(C)c3cccc(-c5cccc6c5-c5ccc(N(c7ccccc7)c7ccccc7)cc5C6(C)C)c3-4)cc21 ; CC1(C)c2ccccc2-c2ccc(N(c3ccc4c(c3)C(C)(C)c3cccc(-c5cccc6c5-c5ccc(N(c7ccccc7)c7ccccc7)cc5C6(C)C)c3-4)c3ccccc3-c3ccccc3)cc21 ; CC1(C)c2cc(N(c3ccccc3)c3ccc(-c4ccccc4)cc3)ccc2-c2c(-c3cccc4c3-c3ccc(N(c5ccccc5)c5cccc6c5C(C)(C)c5ccccc5-6)cc3C4(C)C)cccc21 ; CC1(C)c2cc(N(c3ccccc3)c3cccc(-c4ccccc4)c3)ccc2-c2c(-c3cccc4c3-c3ccc(N(c5ccccc5)c5cccc6c5C(C)(C)c5ccccc5-6)cc3C4(C)C)cccc21]

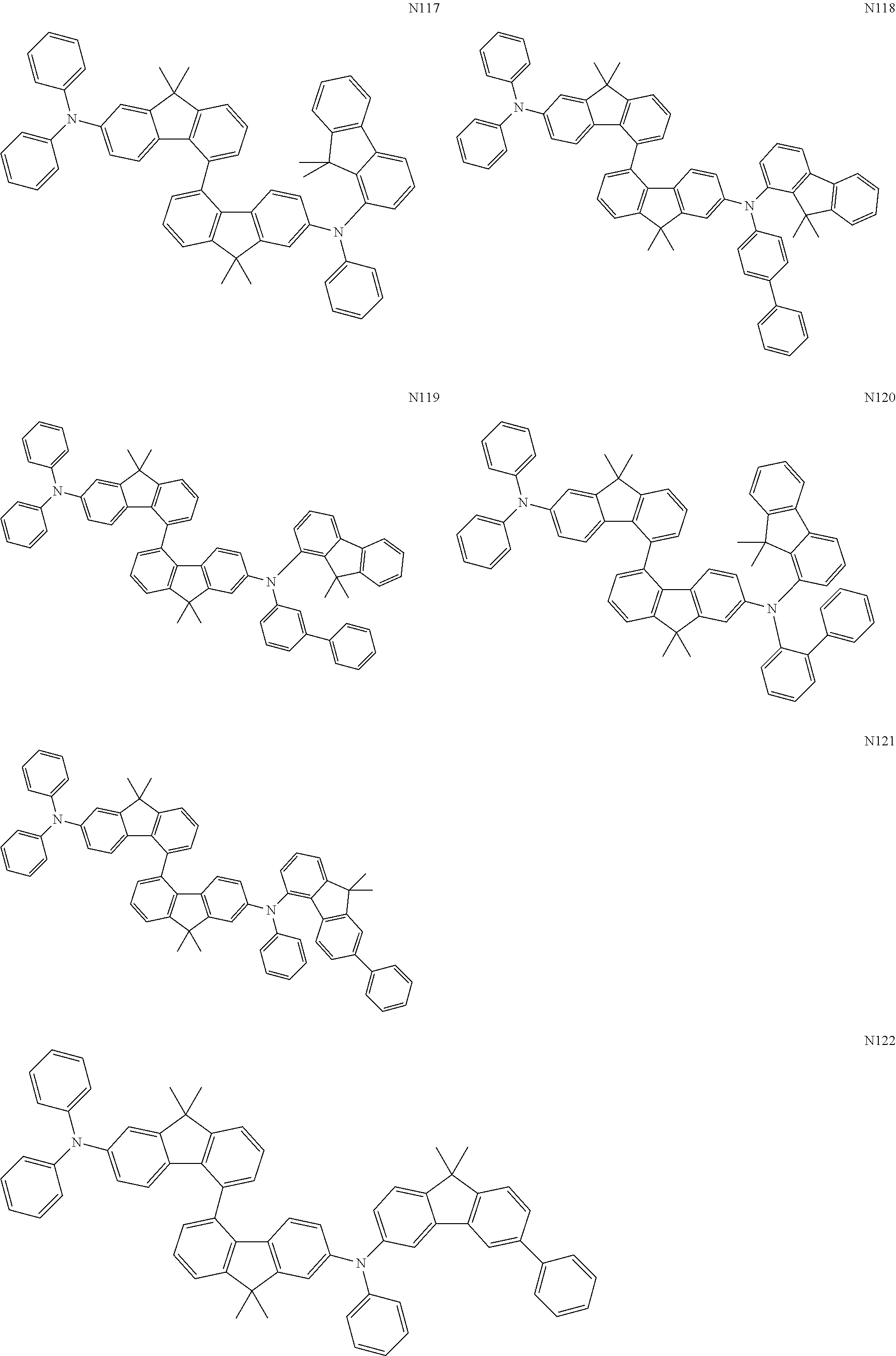

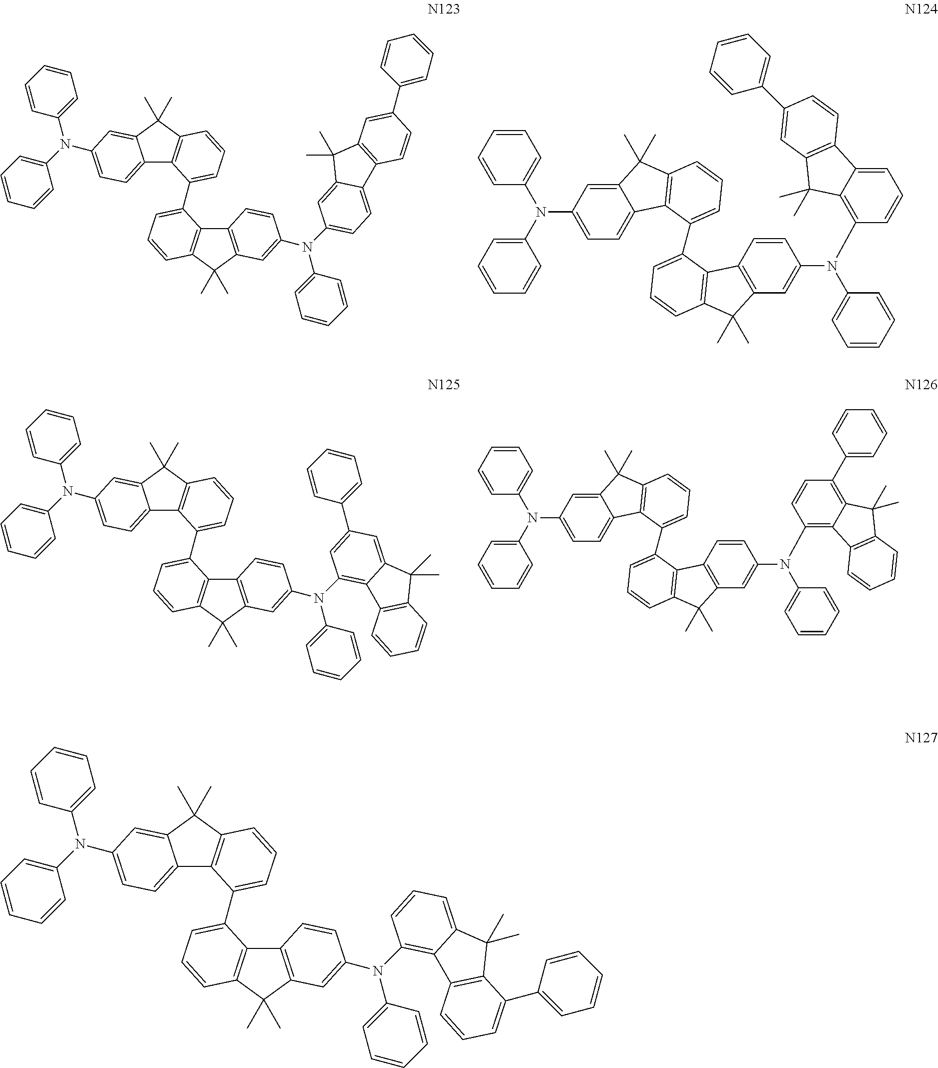

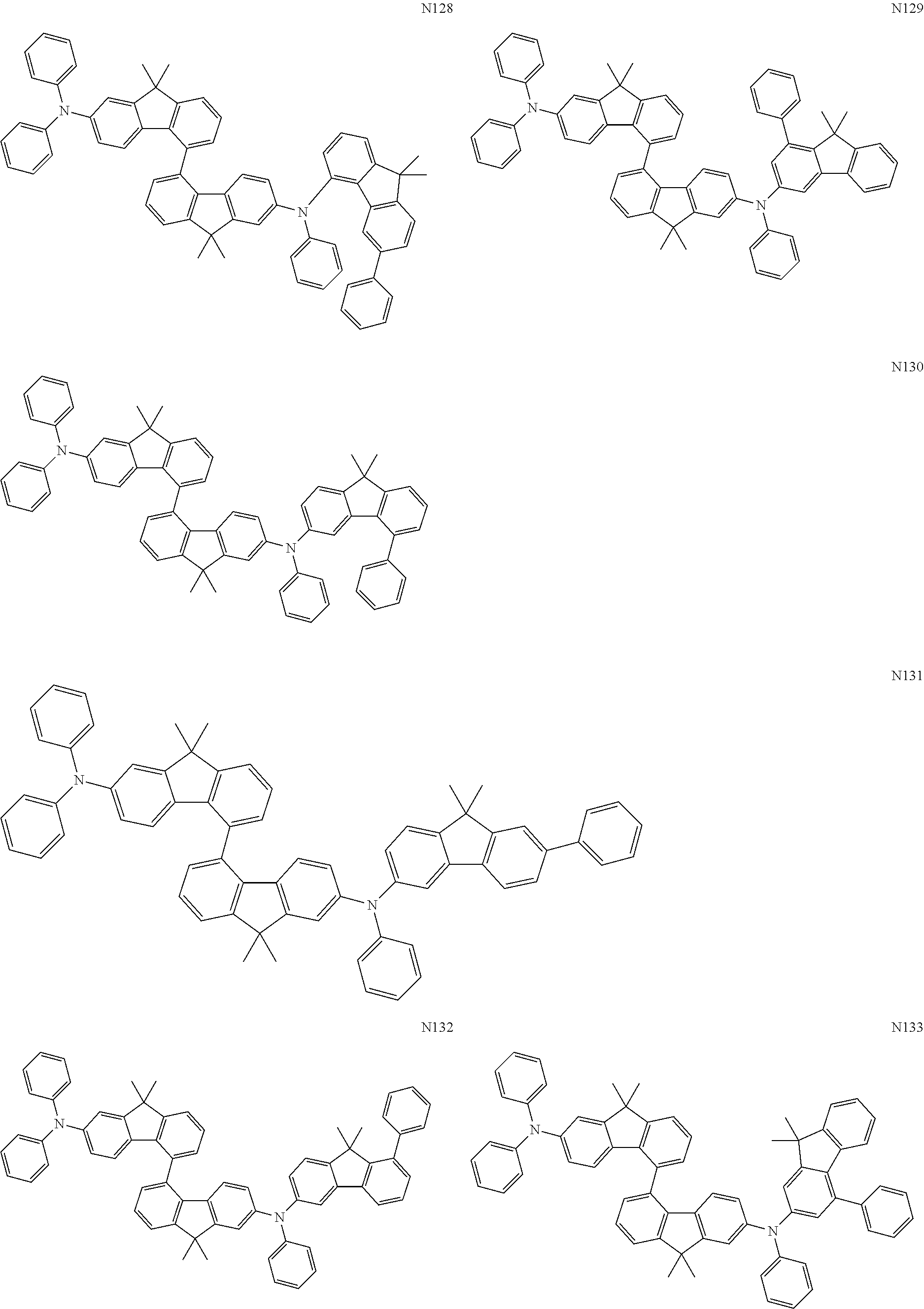

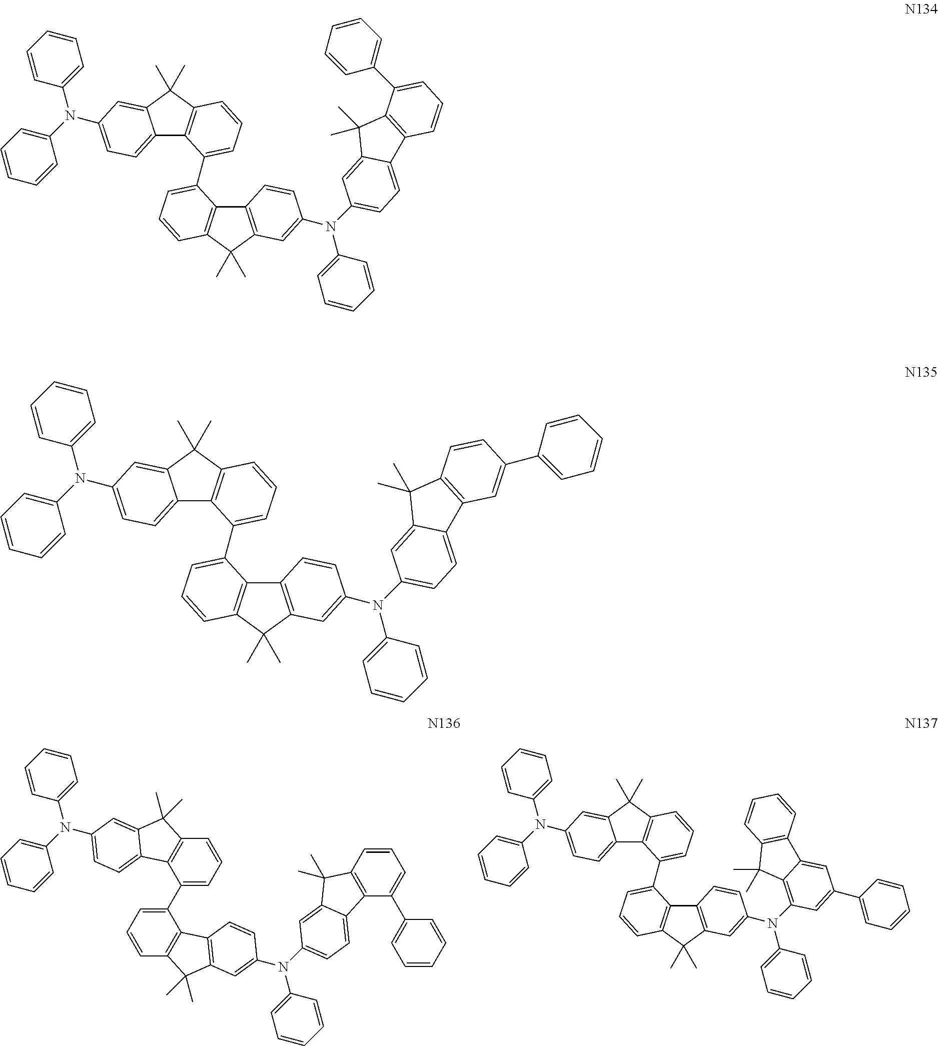

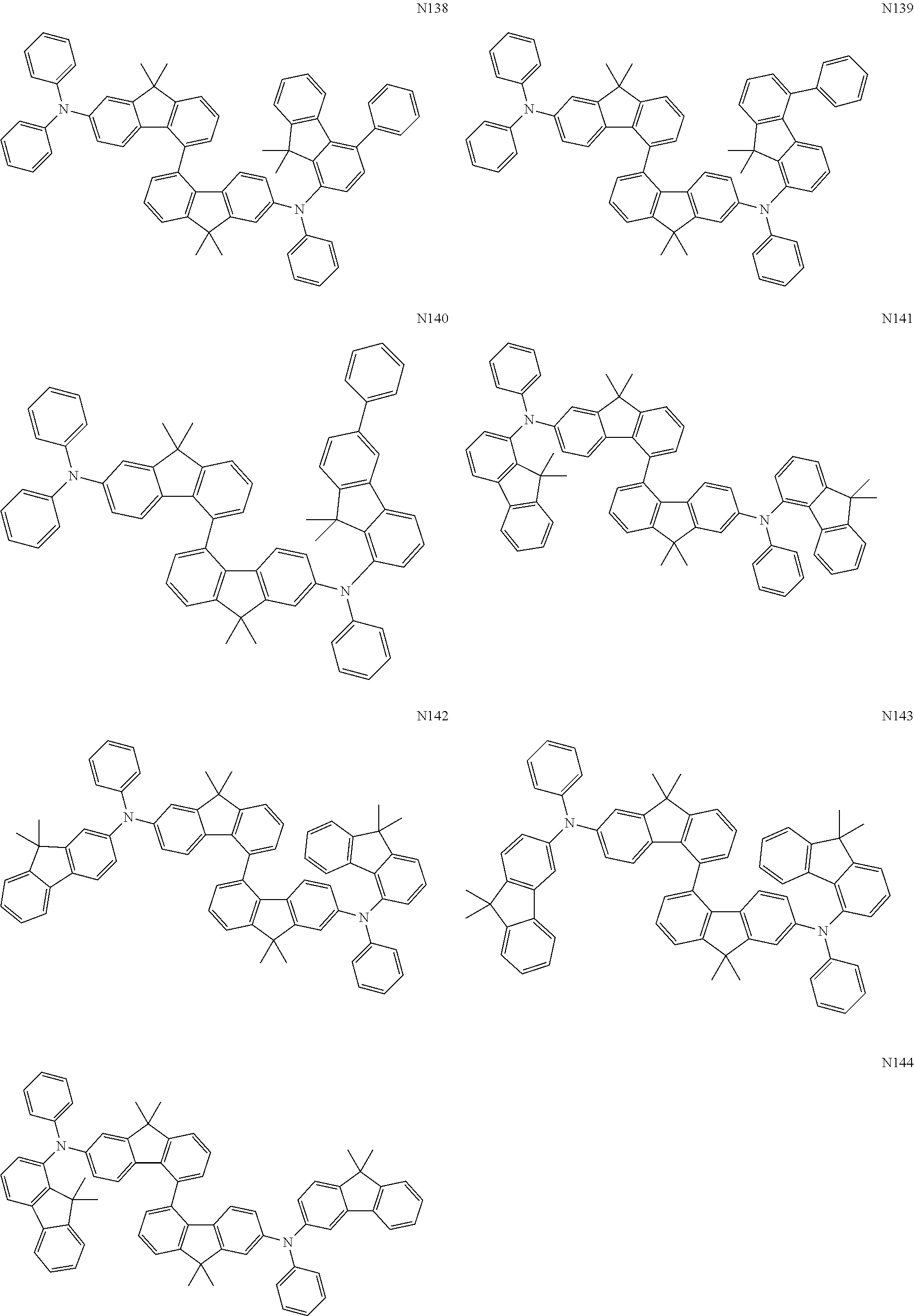

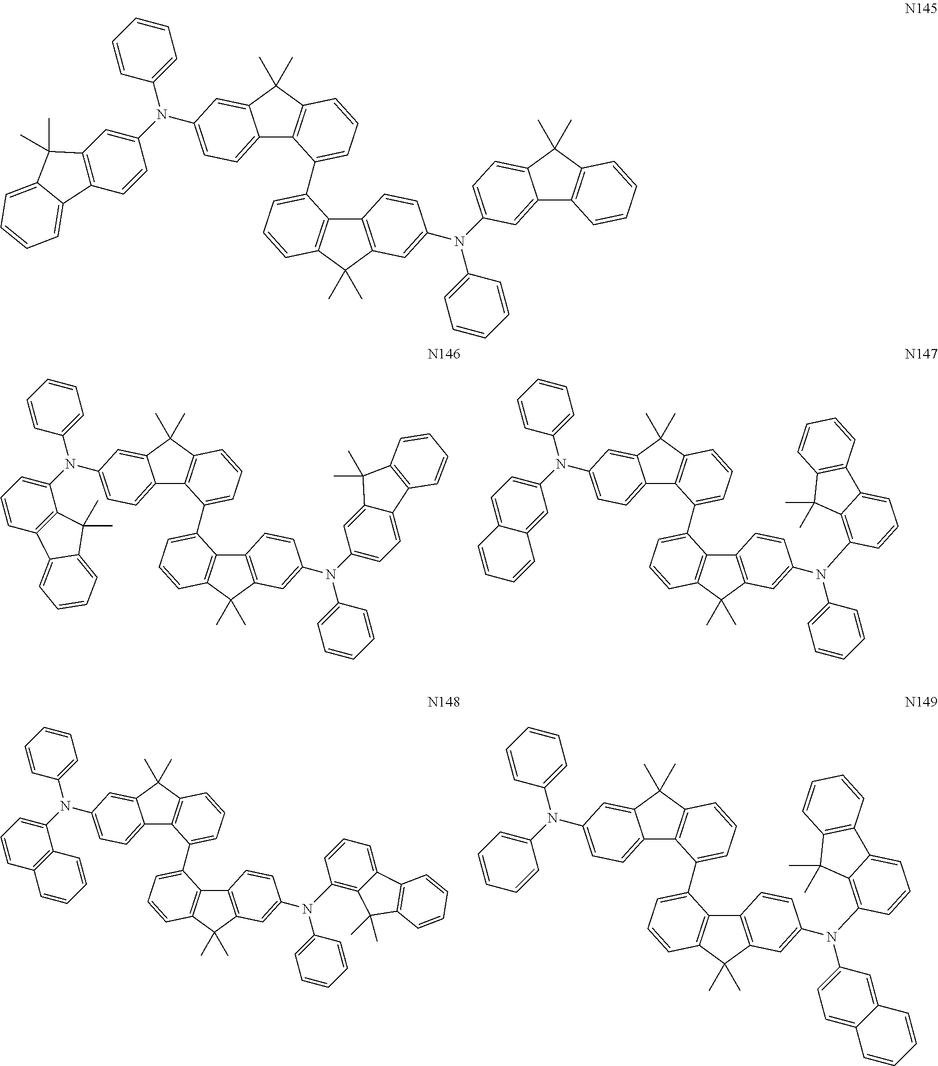

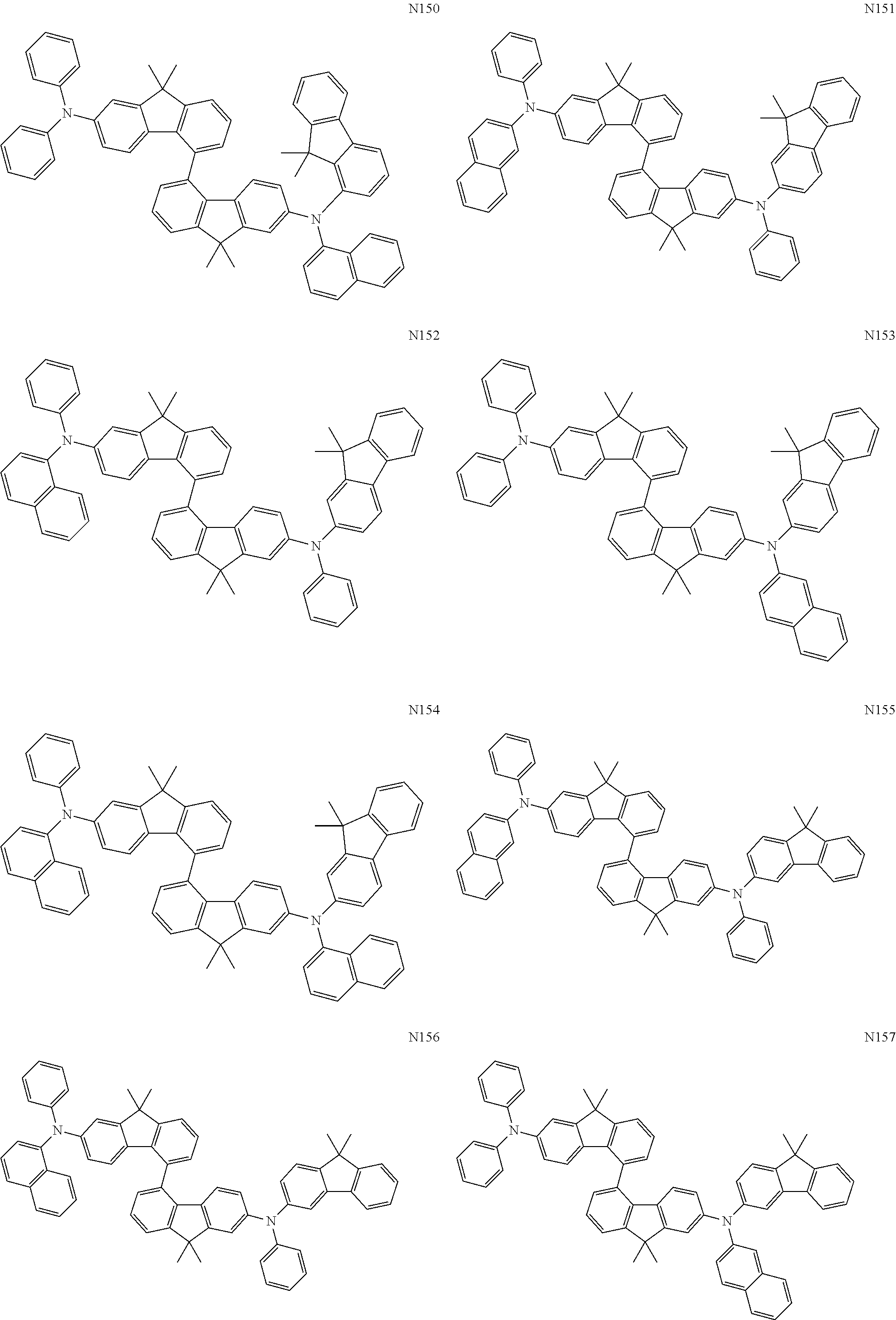

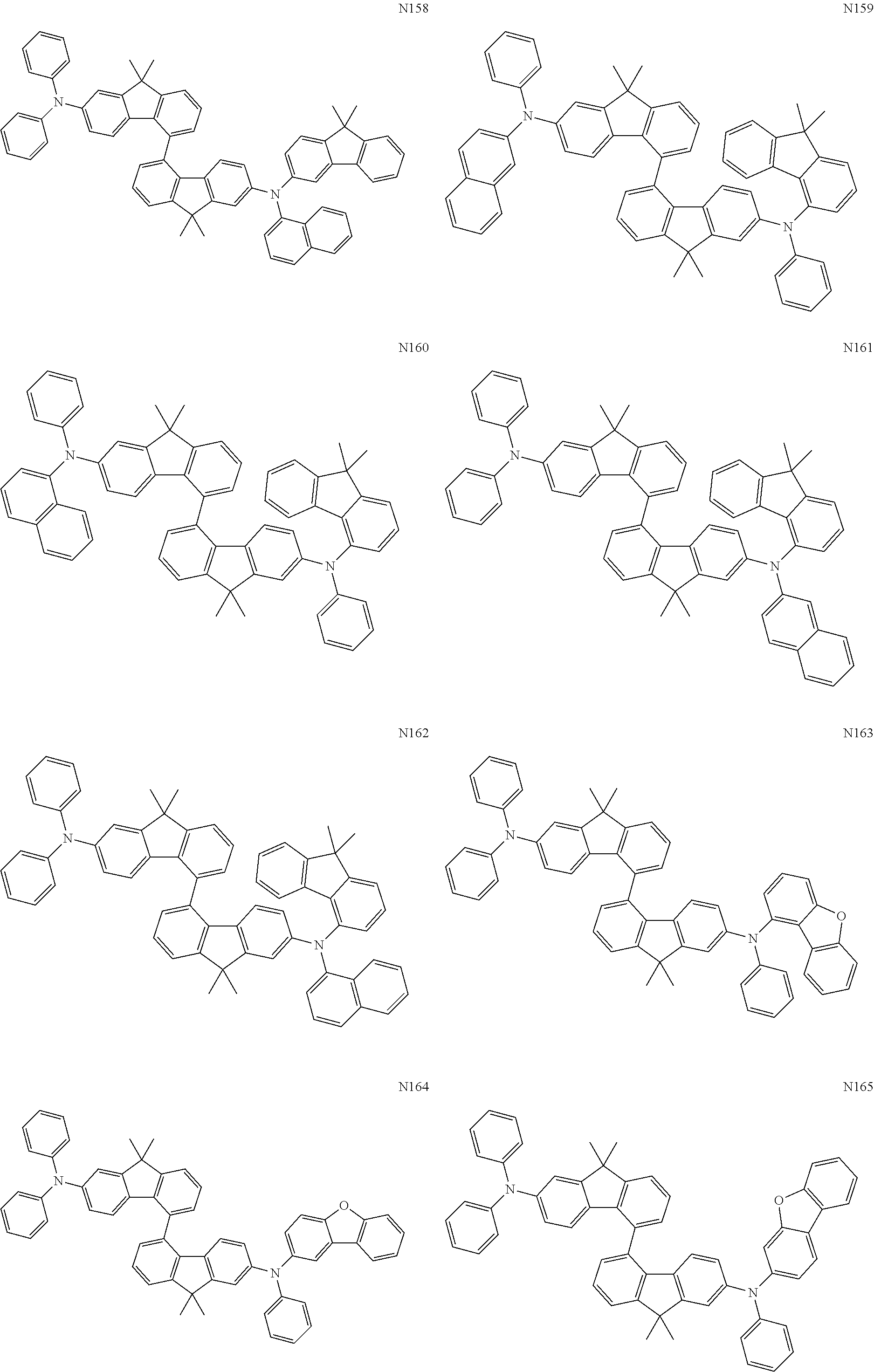

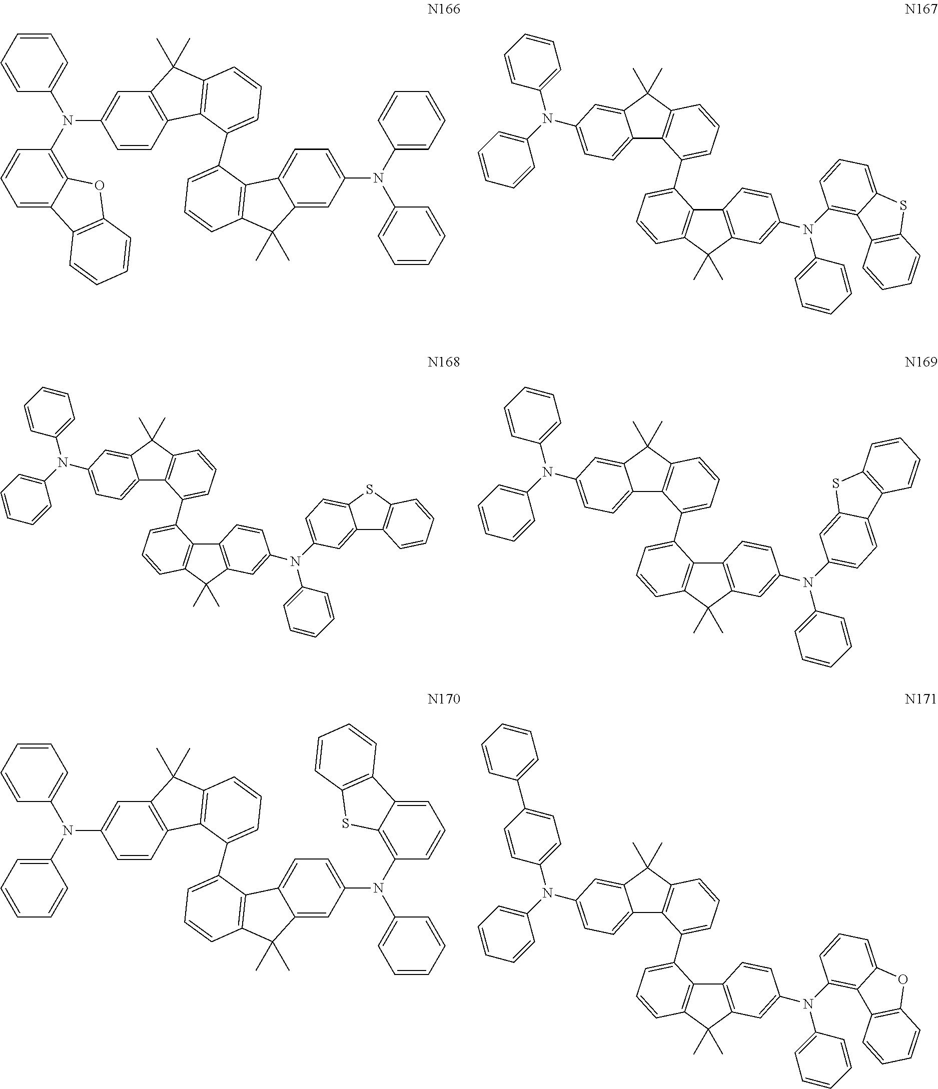

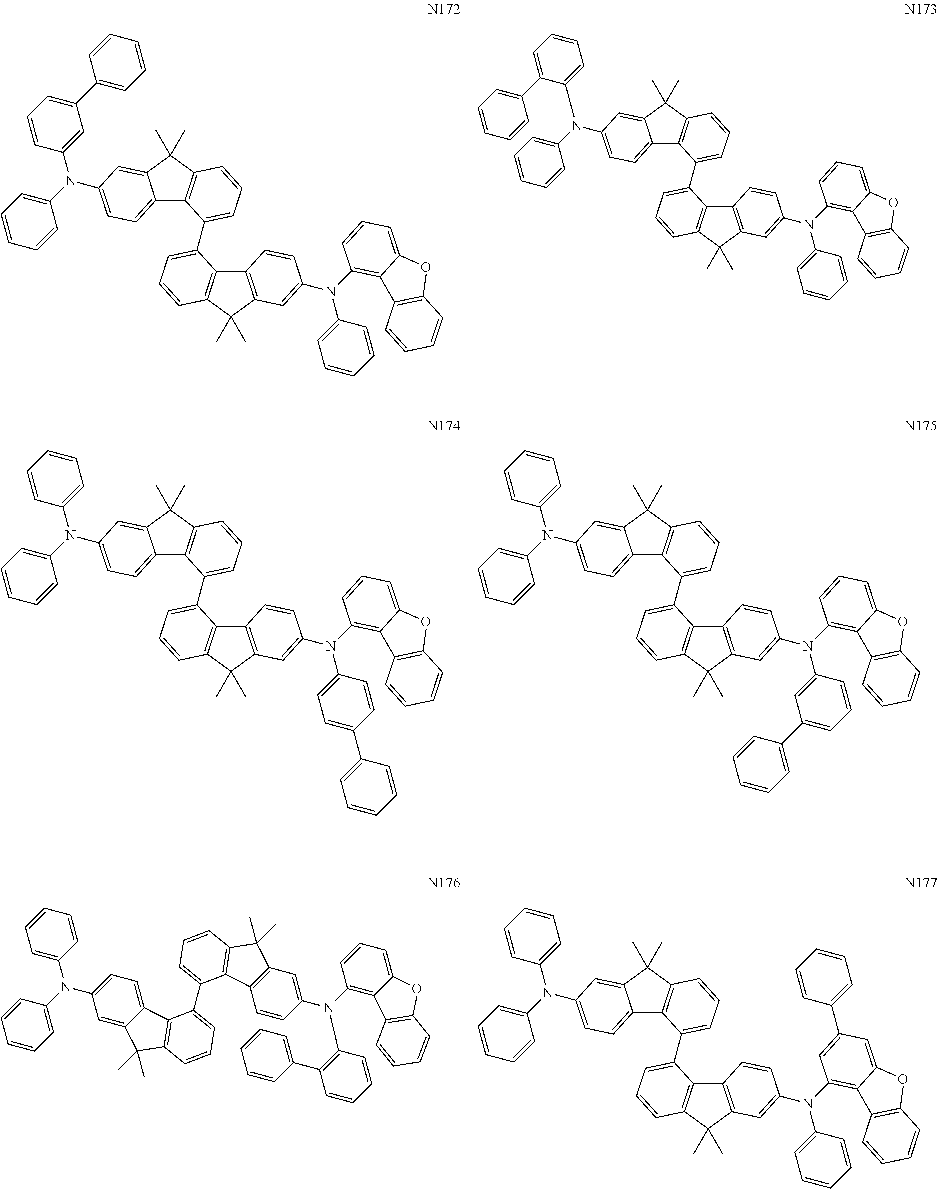

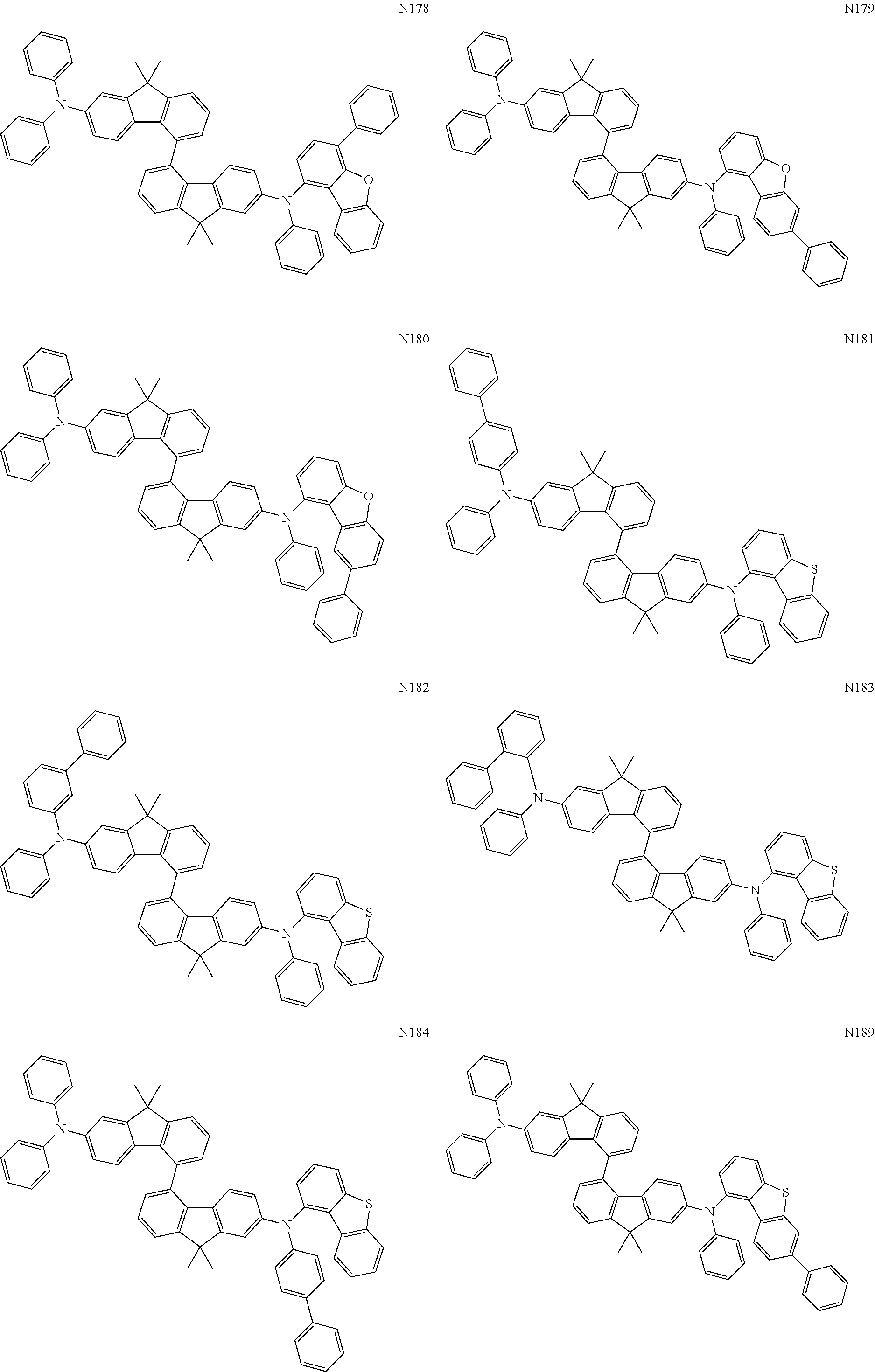

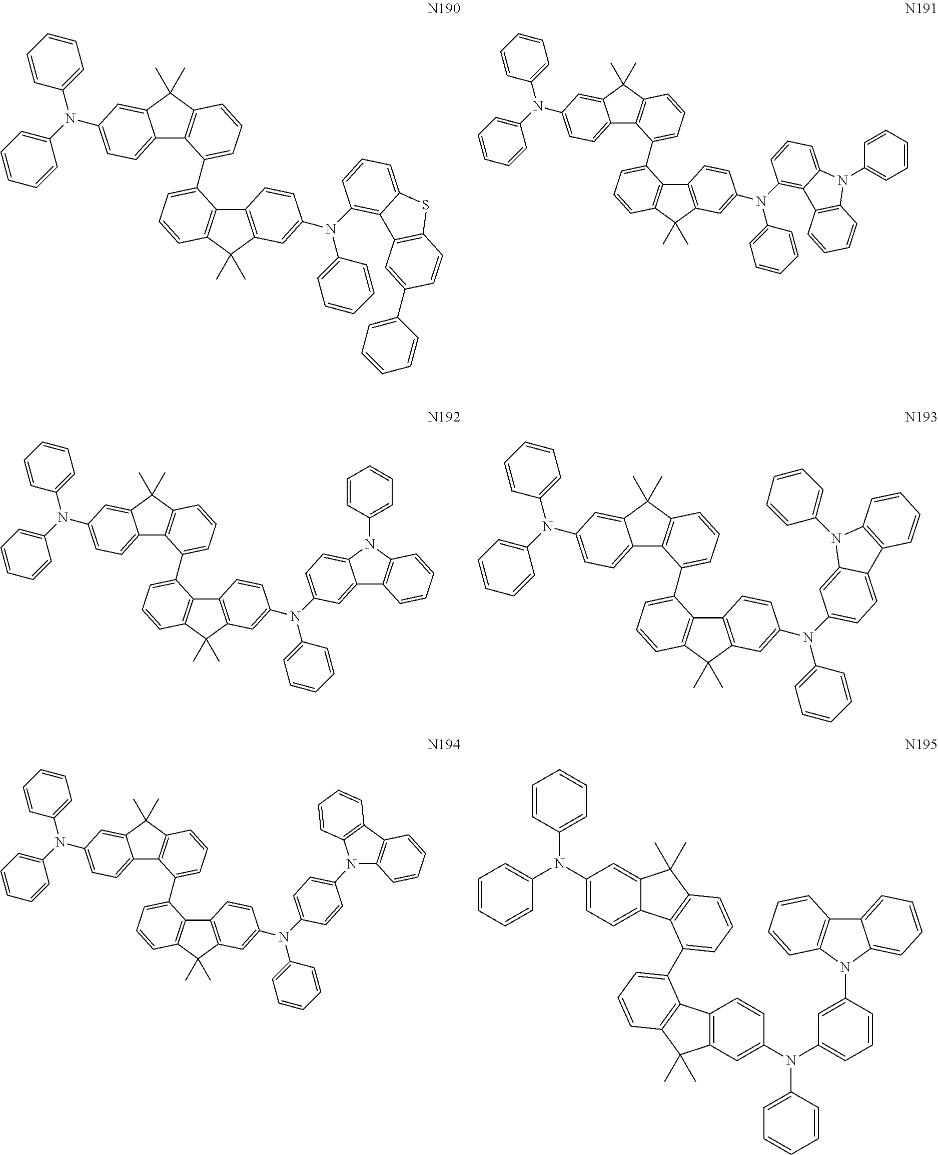

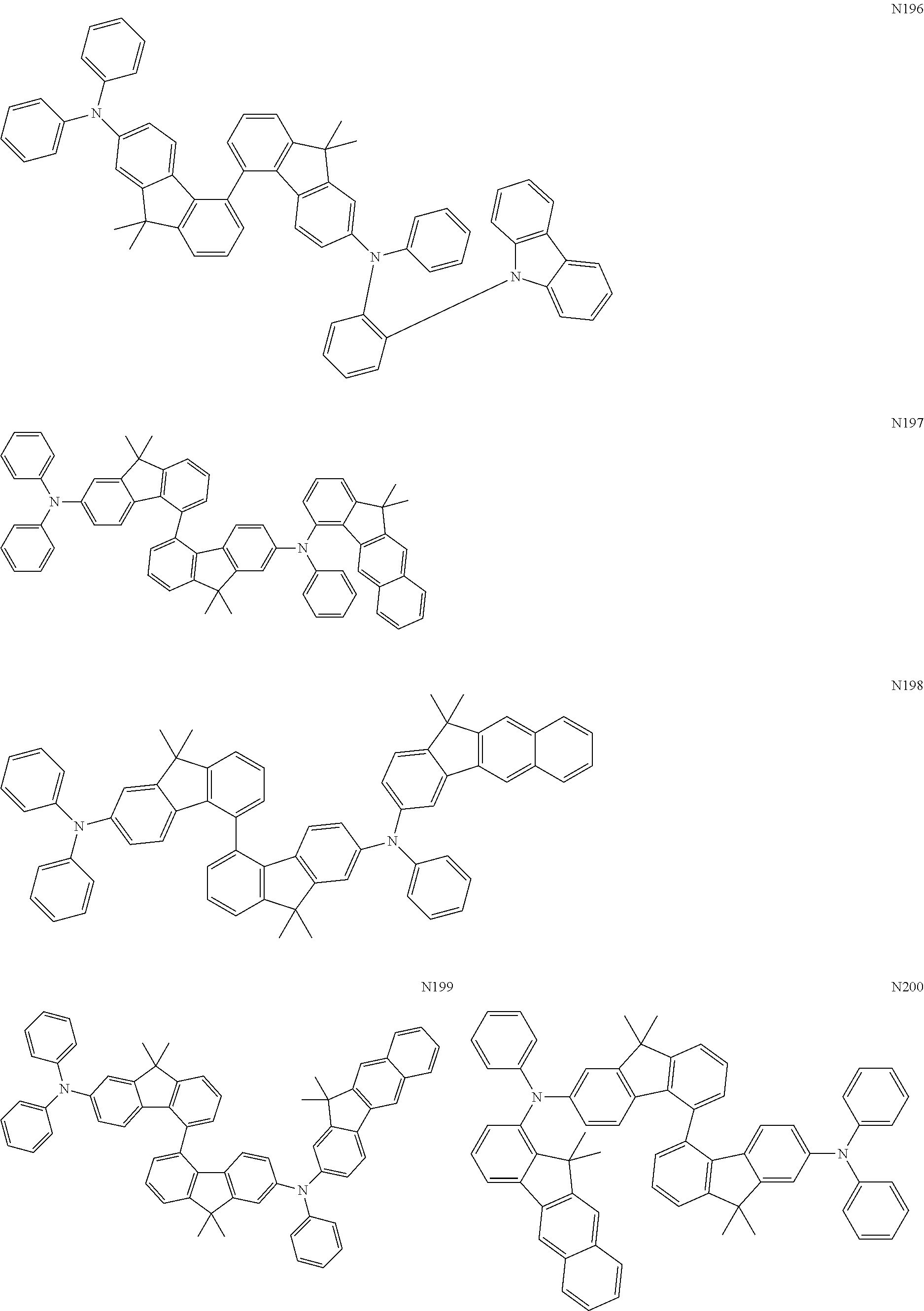

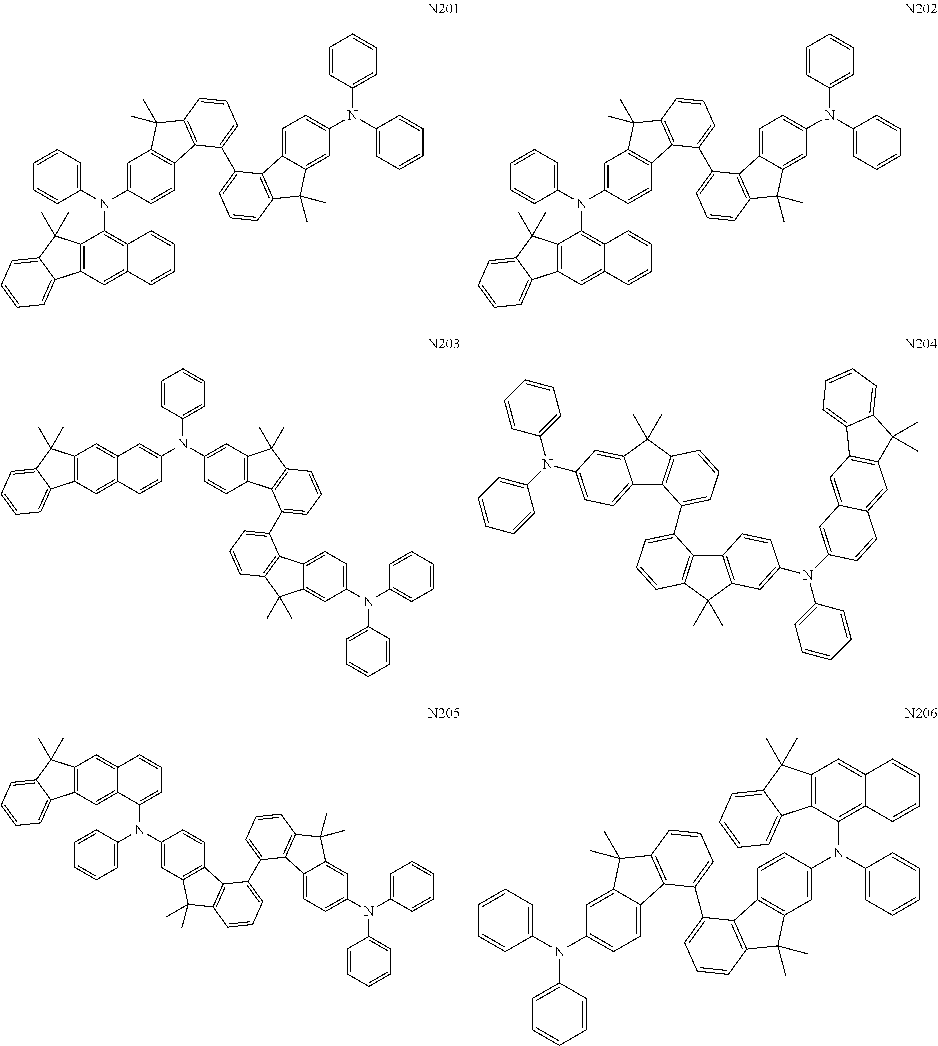

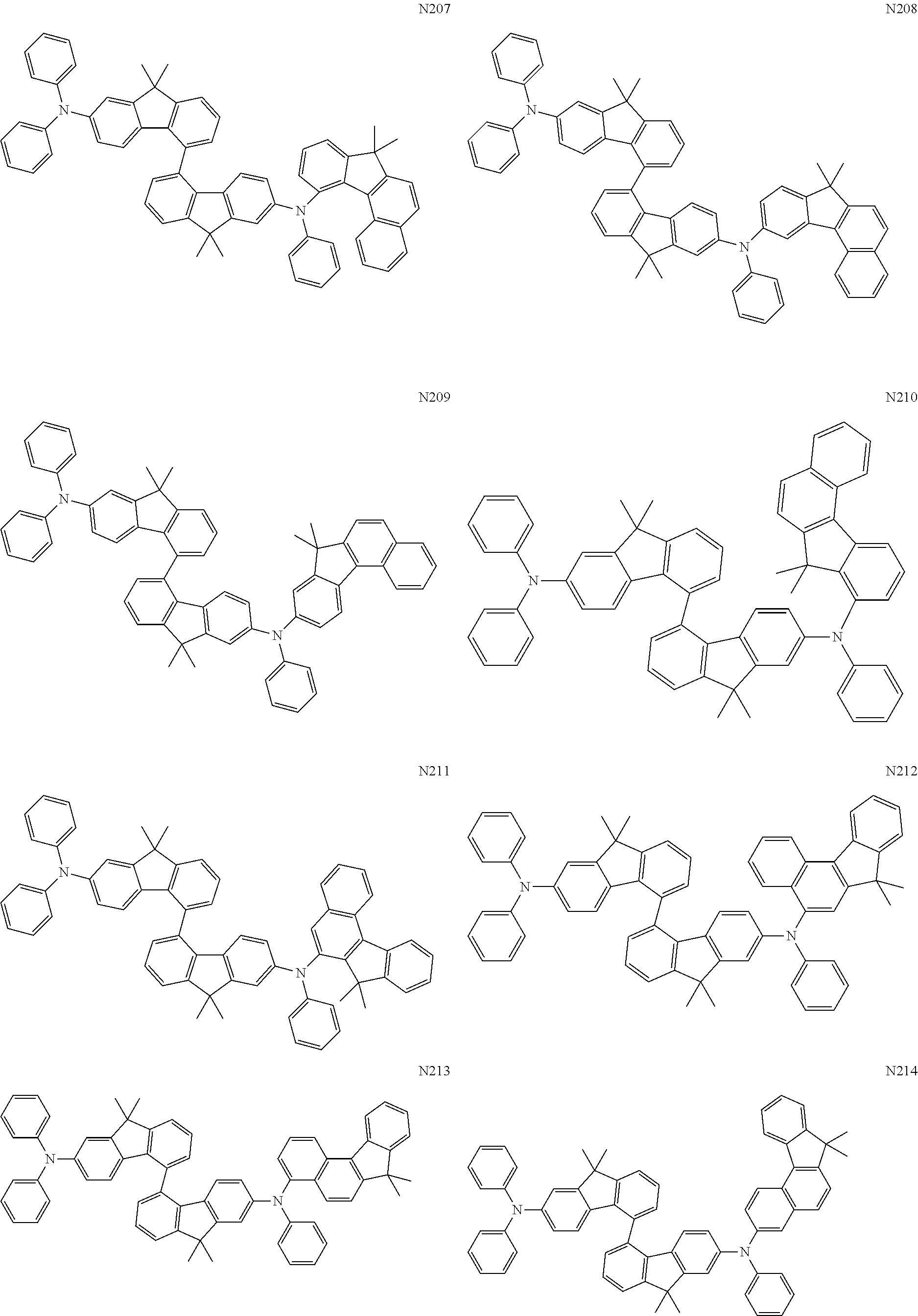

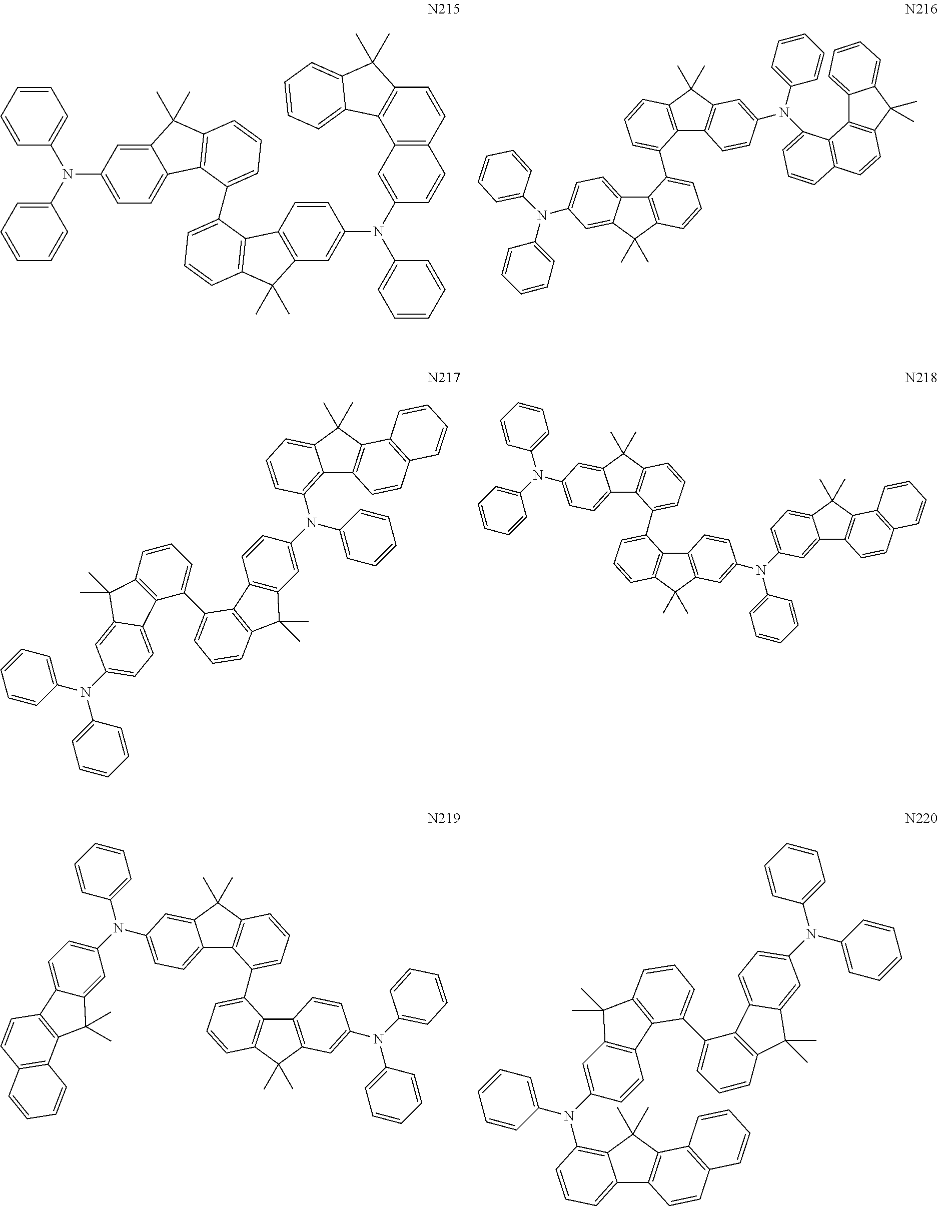

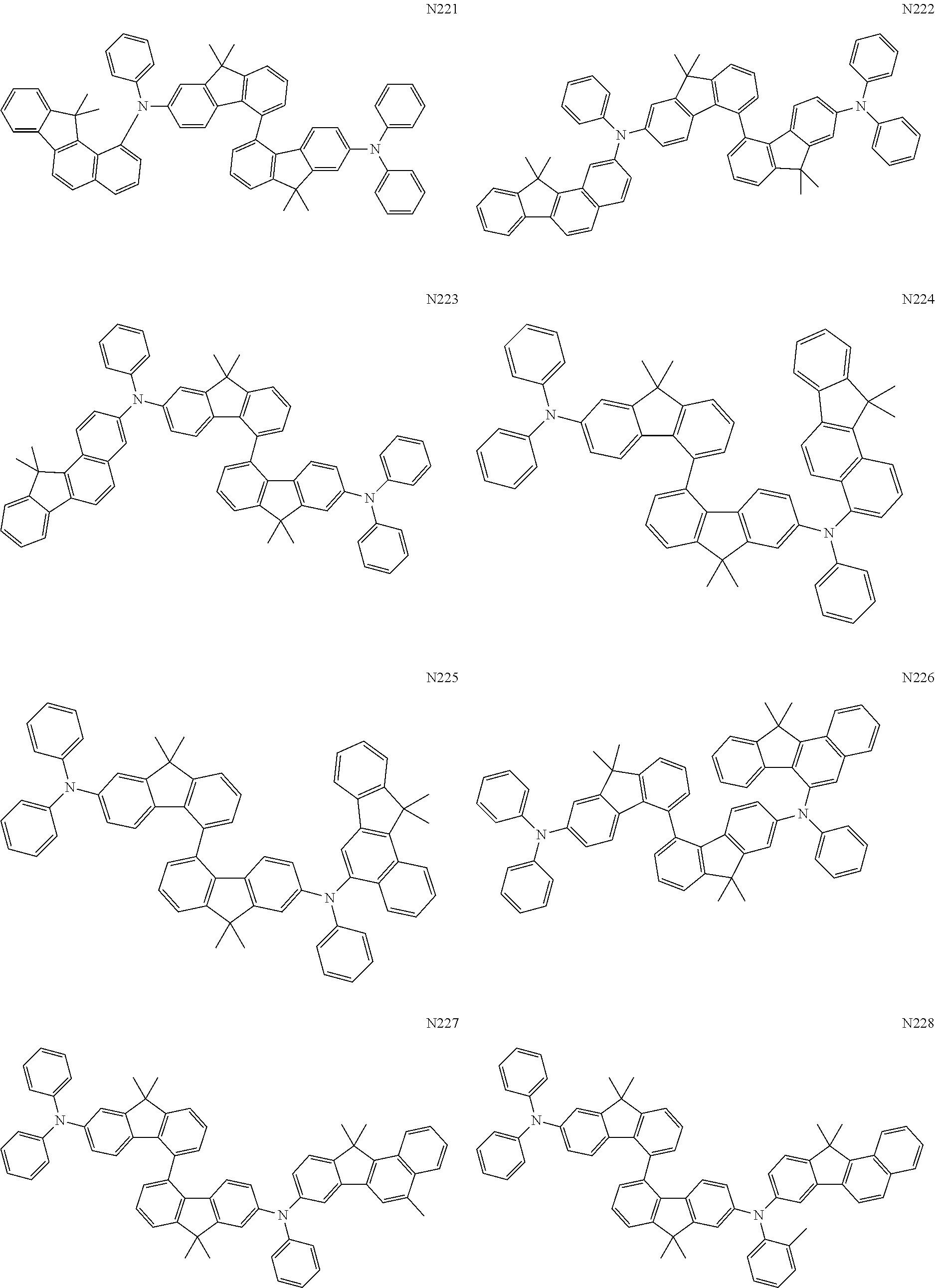

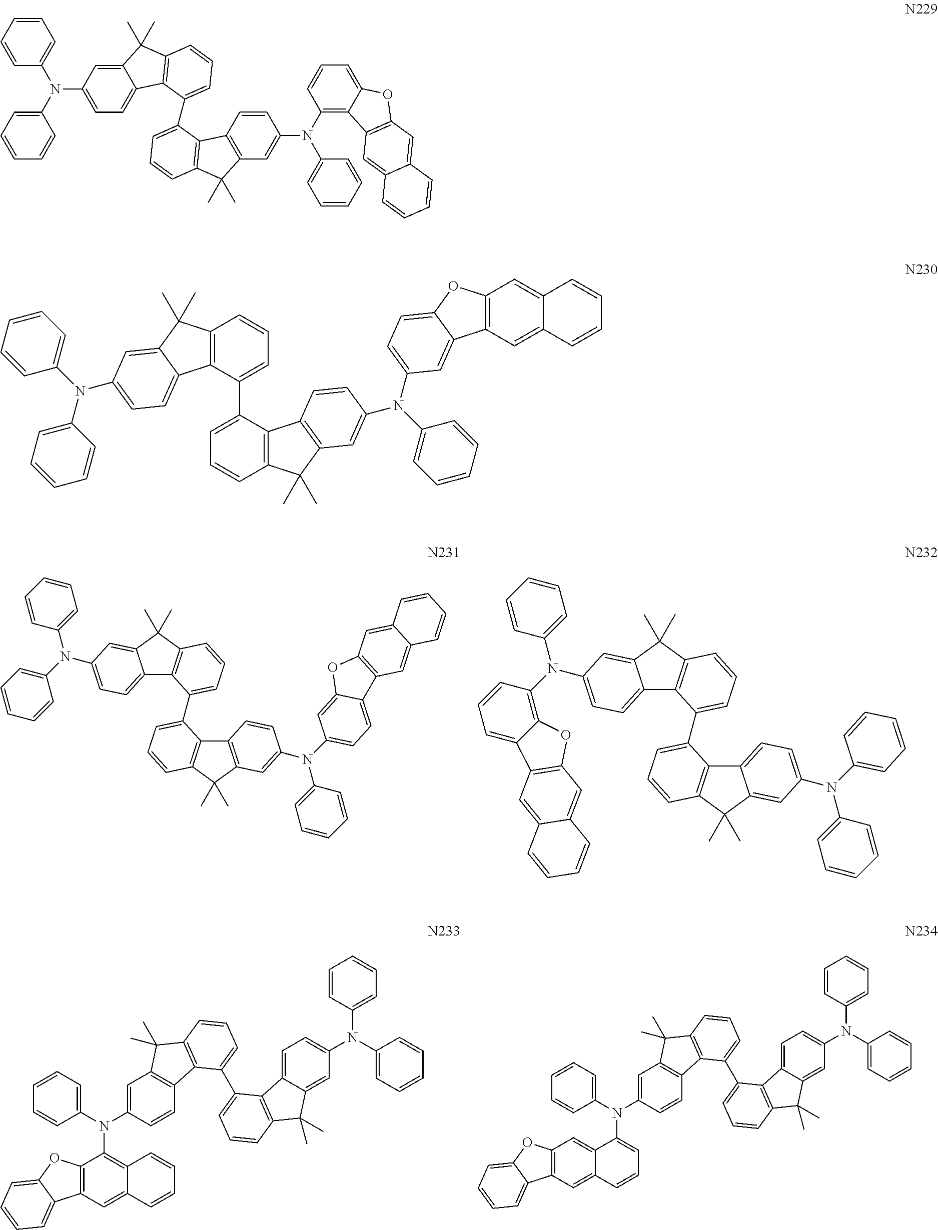

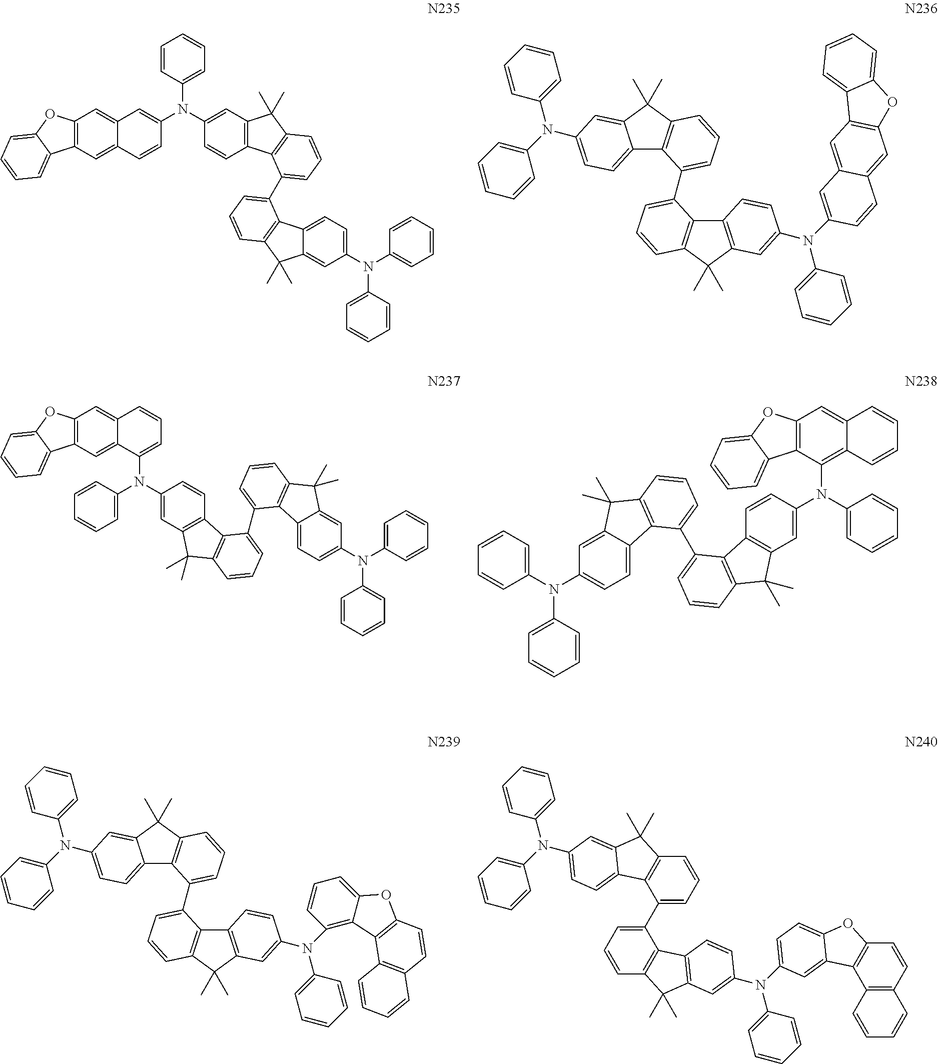

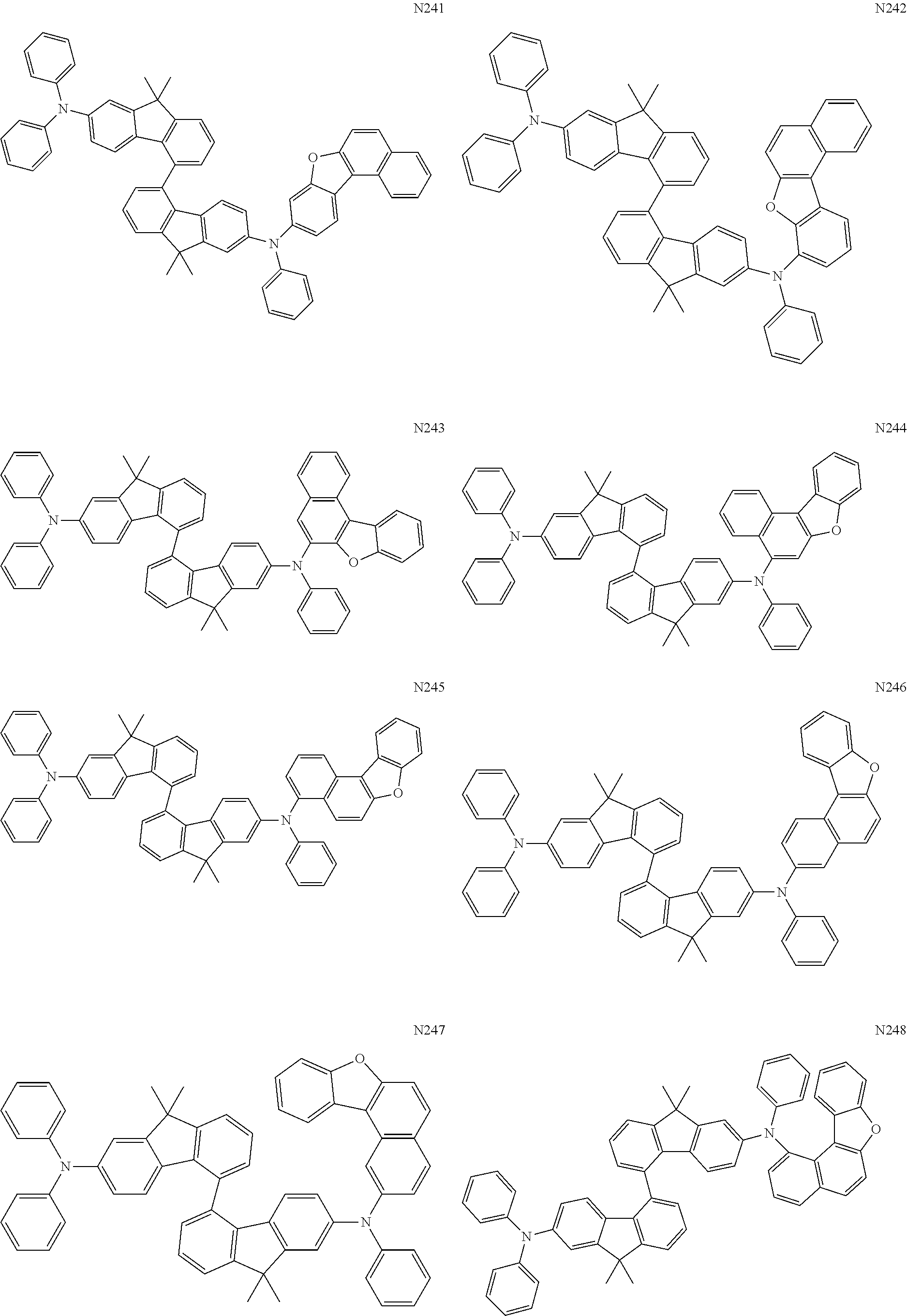

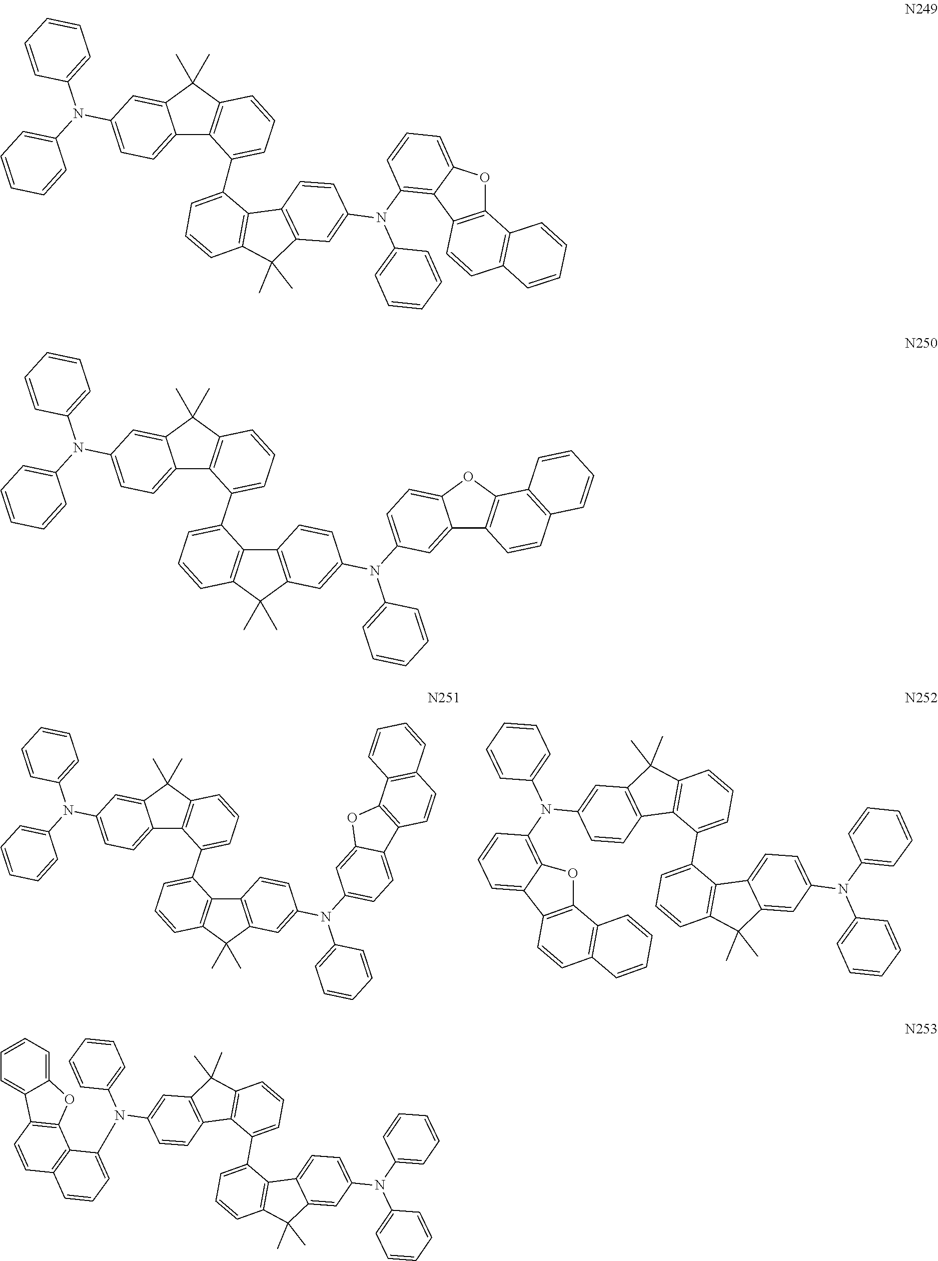

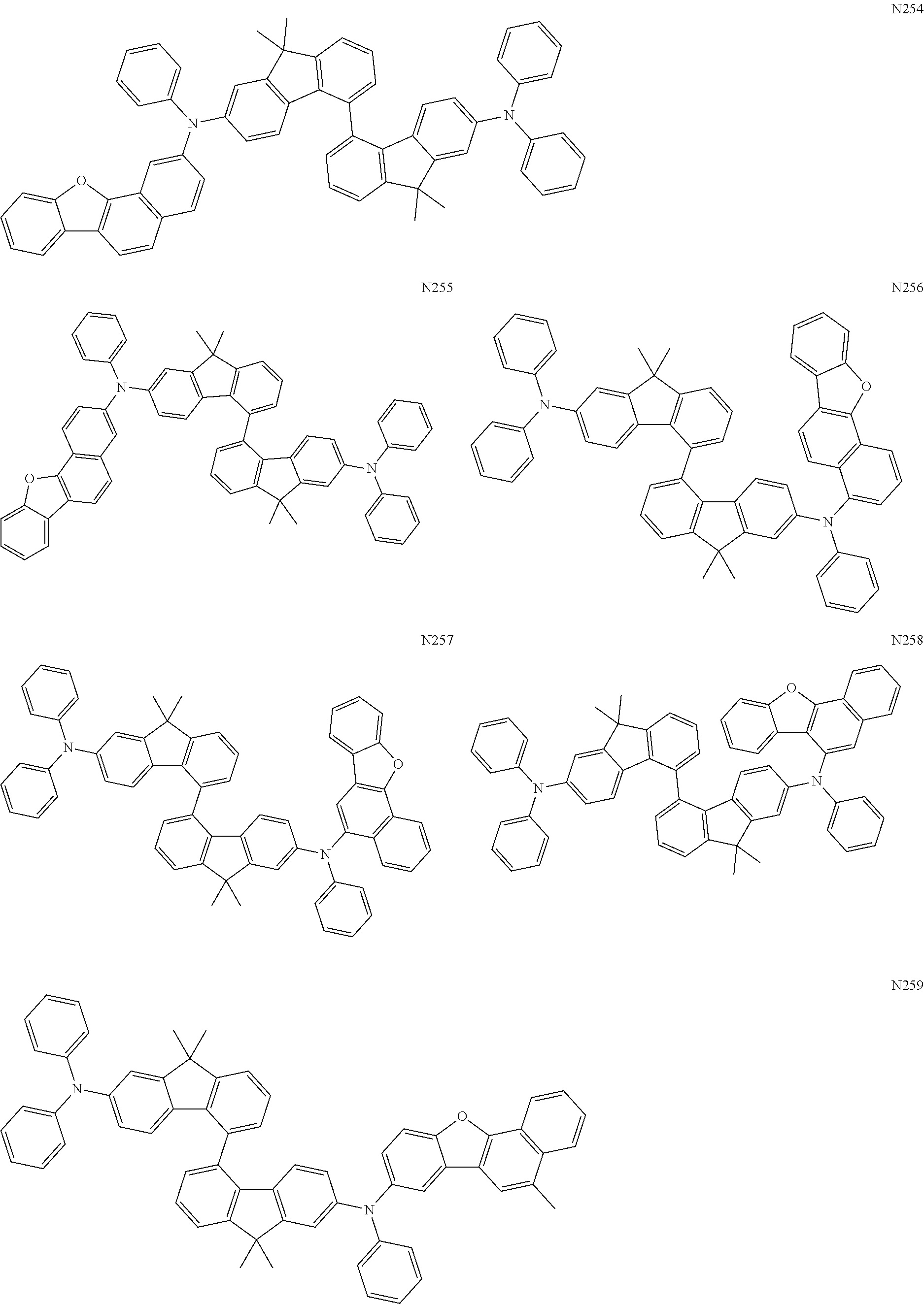

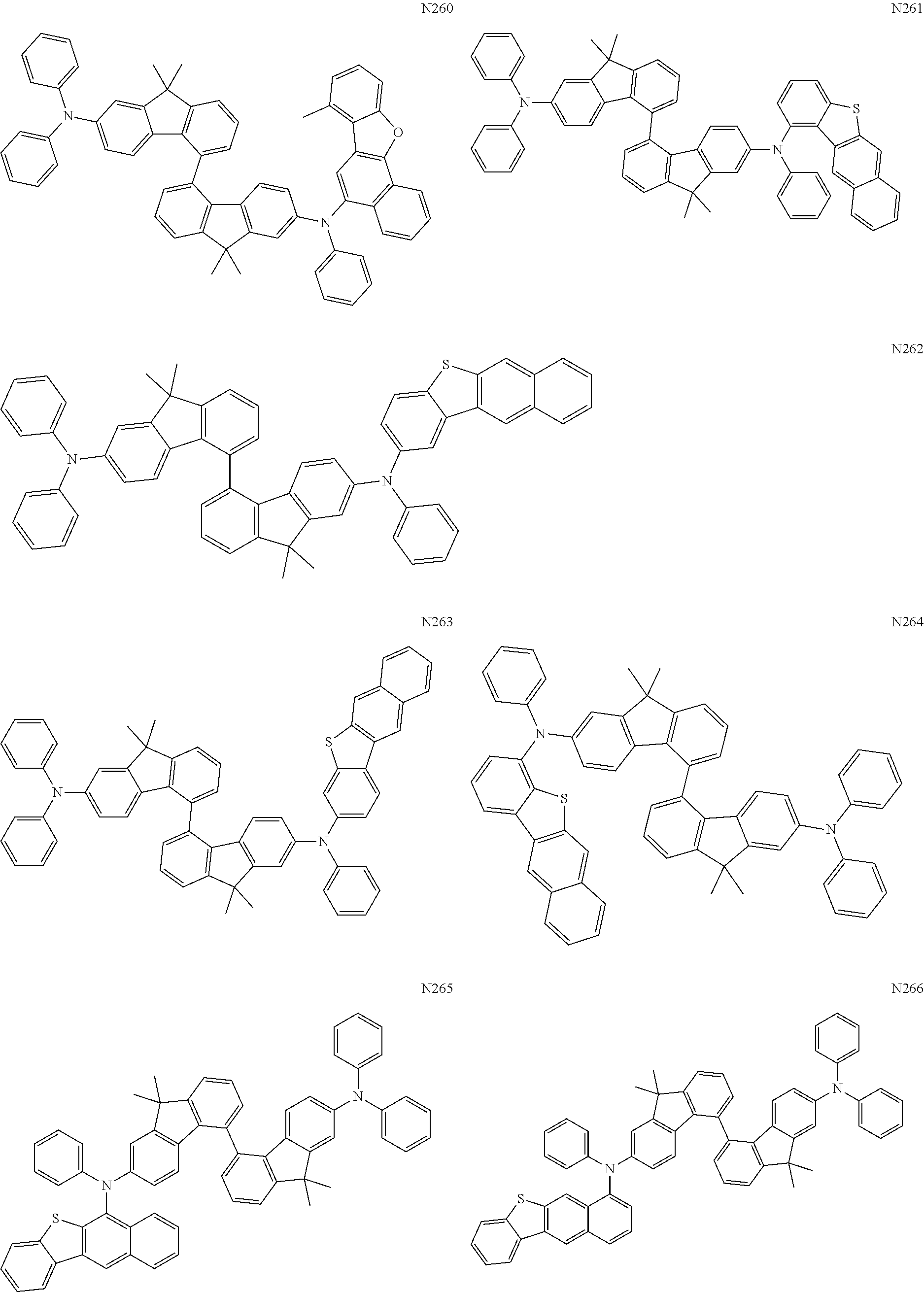

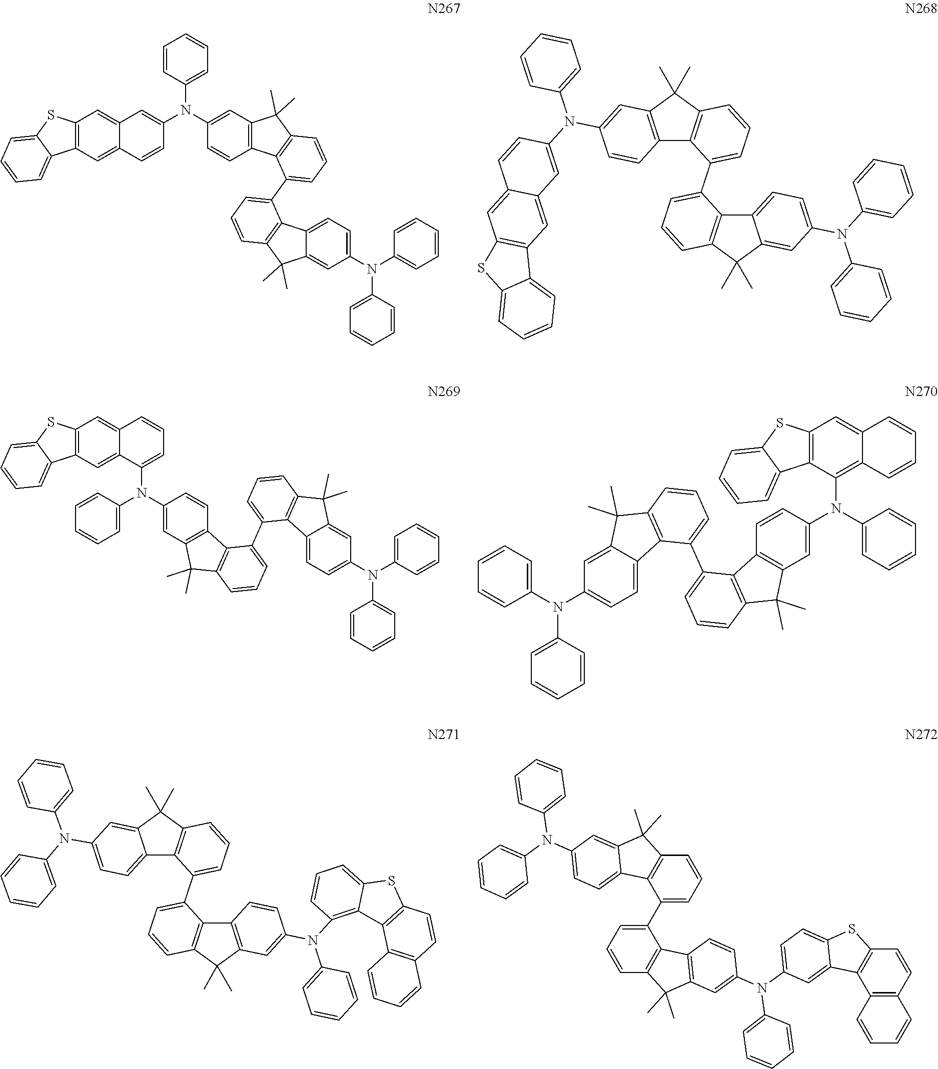

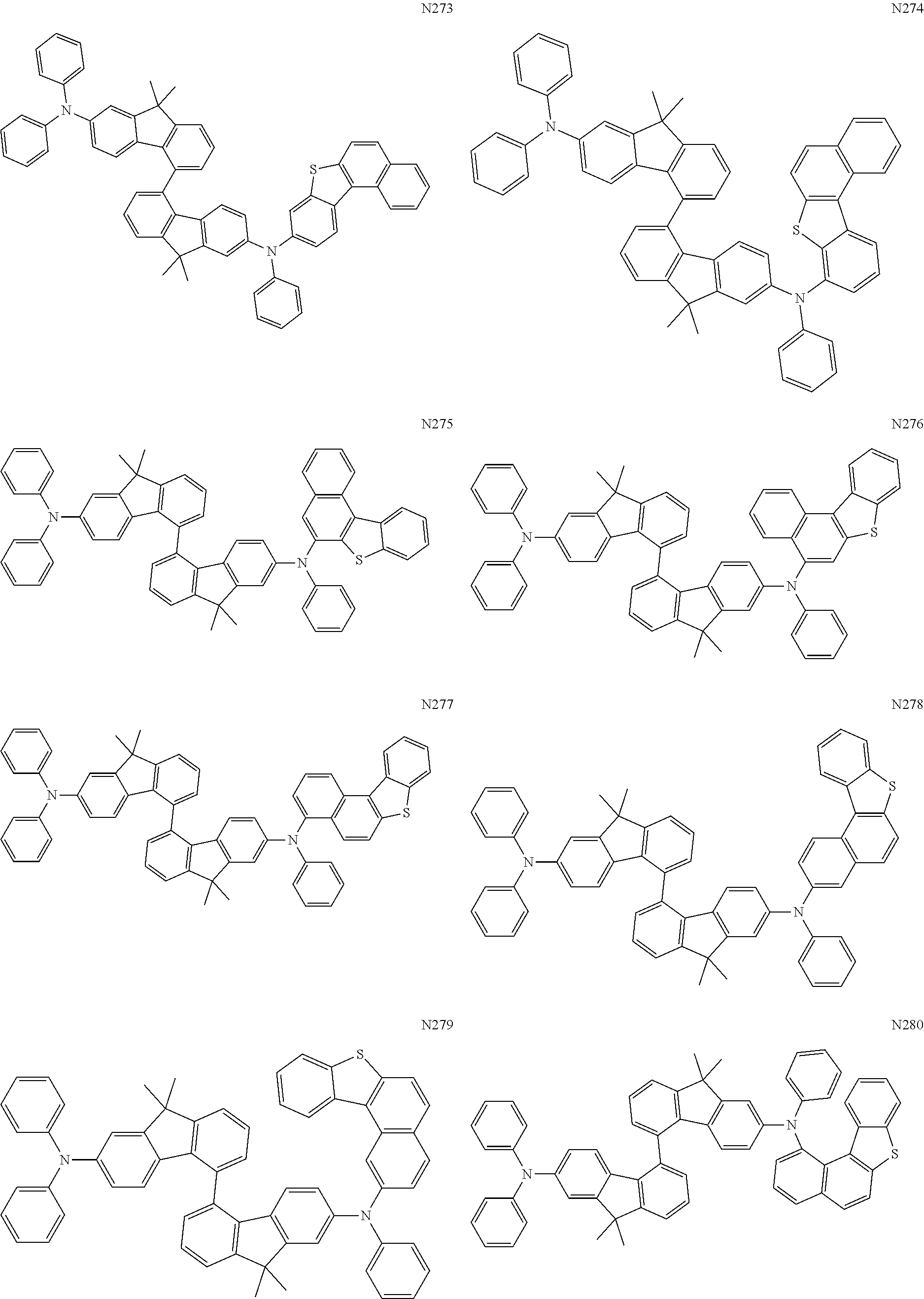

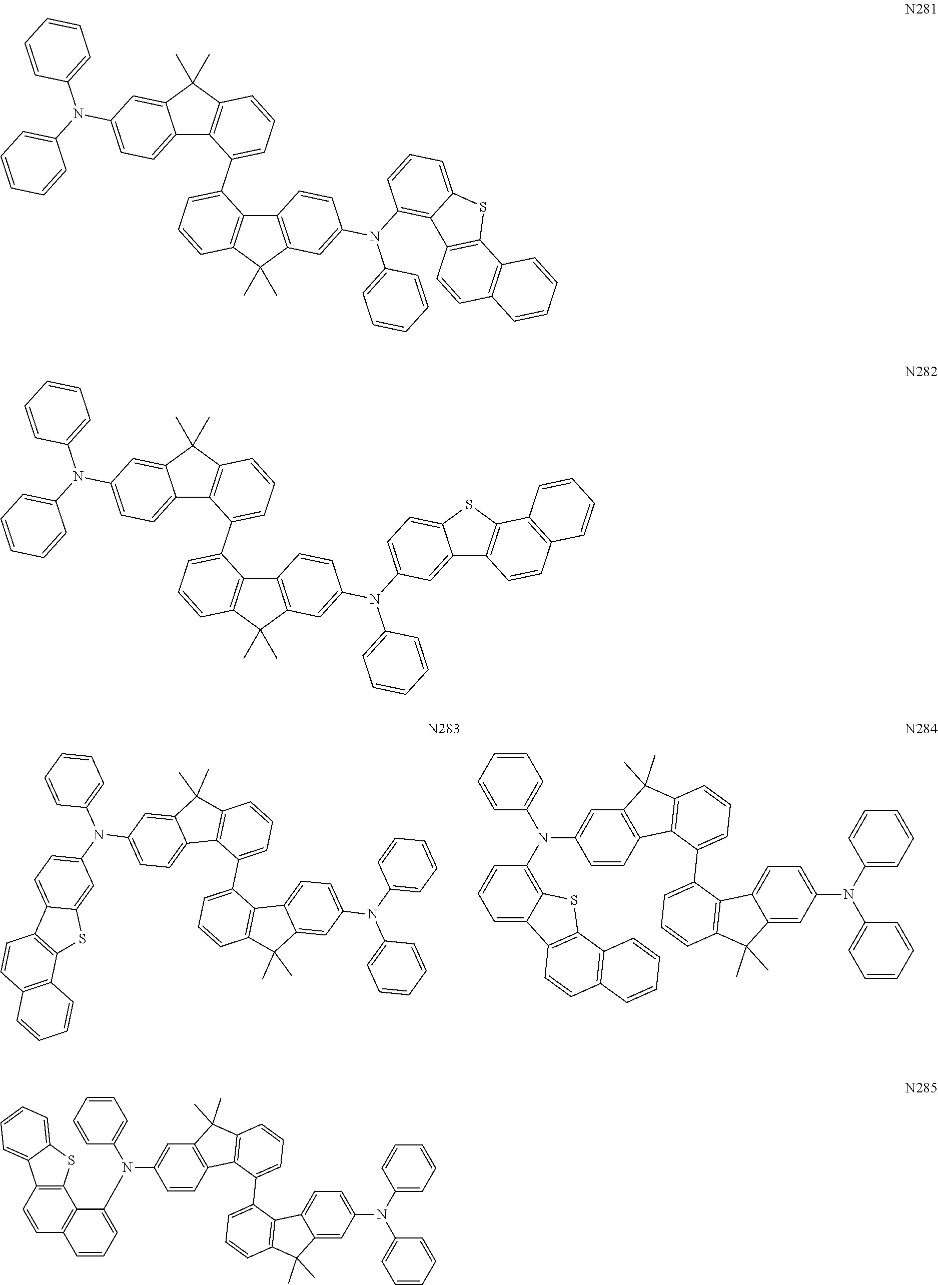

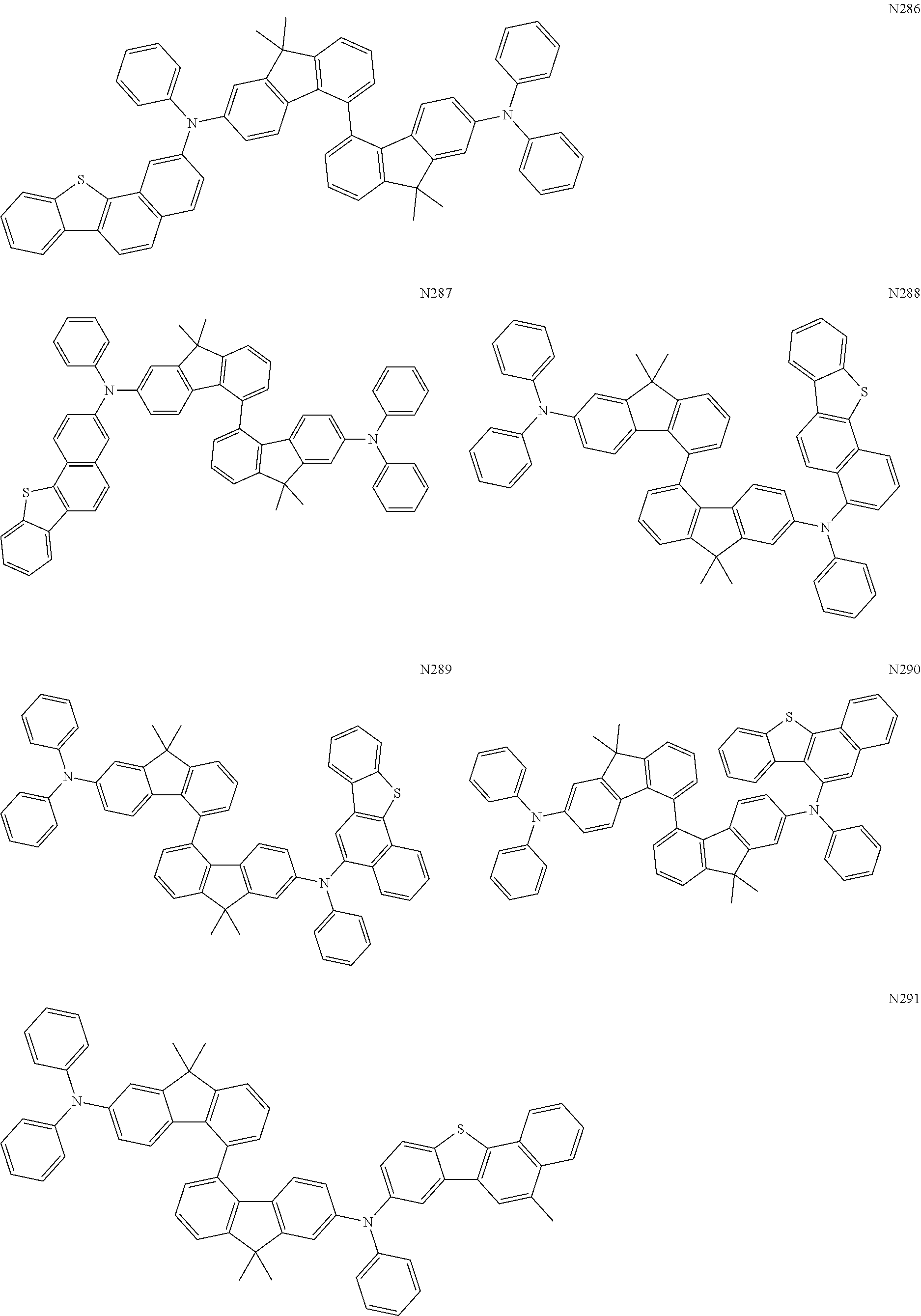

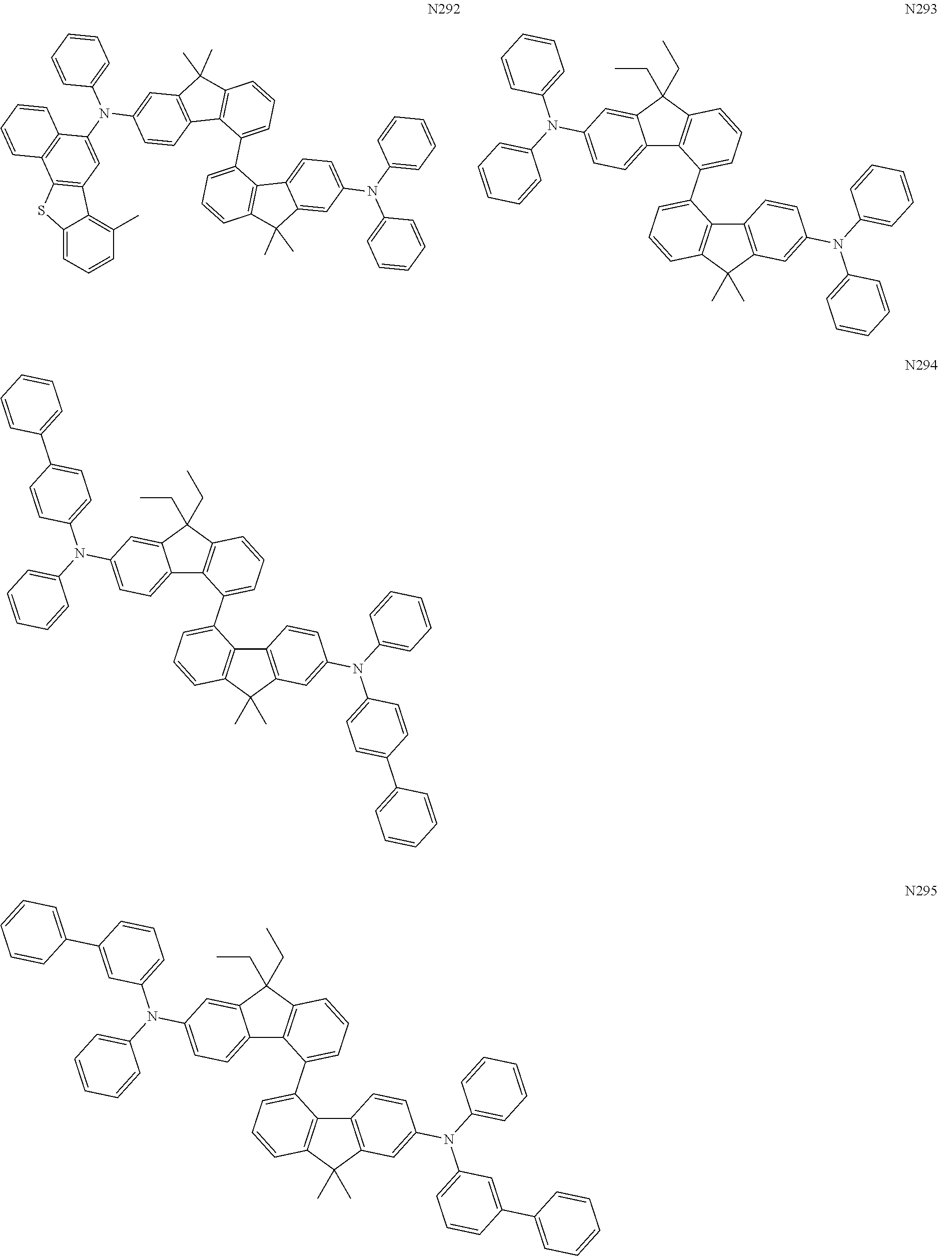 [SMILES: Cc1cccc2sc3c4ccccc4c(N(c4ccccc4)c4ccc5c(c4)C(C)(C)c4cccc(-c6cccc7c6-c6ccc(N(c8ccccc8)c8ccccc8)cc6C7(C)C)c4-5)cc3c12 ; CCC1(CC)c2cc(N(c3ccccc3)c3ccccc3)ccc2-c2c(-c3cccc4c3-c3ccc(N(c5ccccc5)c5ccccc5)cc3C4(C)C)cccc21 ; CCC1(CC)c2cc(N(c3ccccc3)c3ccc(-c4ccccc4)cc3)ccc2-c2c(-c3cccc4c3-c3ccc(N(c5ccccc5)c5ccc(-c6ccccc6)cc5)cc3C4(C)C)cccc21 ; CCC1(CC)c2cc(N(c3ccccc3)c3cccc(-c4ccccc4)c3)ccc2-c2c(-c3cccc4c3-c3ccc(N(c5ccccc5)c5cccc(-c6ccccc6)c5)cc3C4(C)C)cccc21]

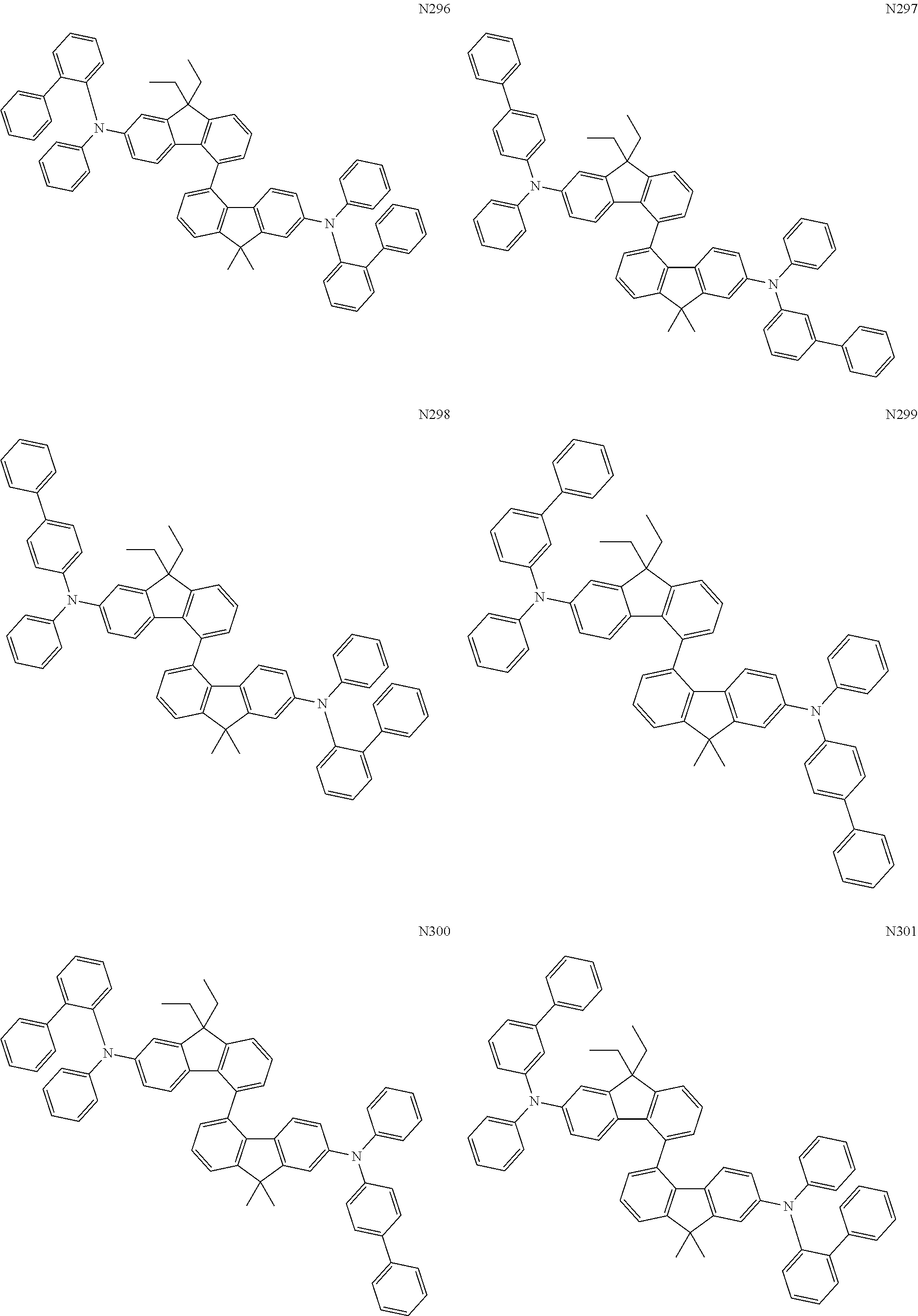

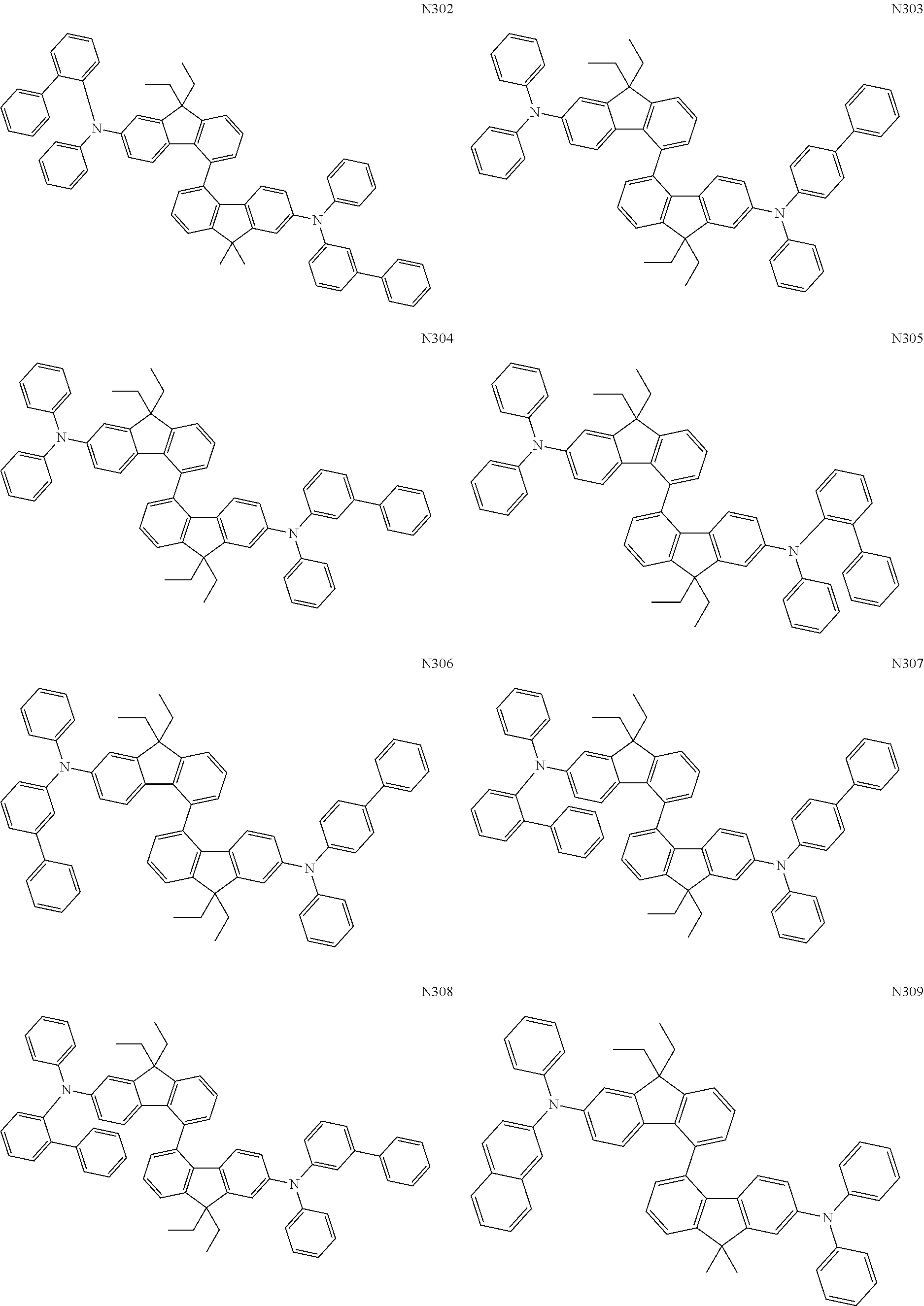

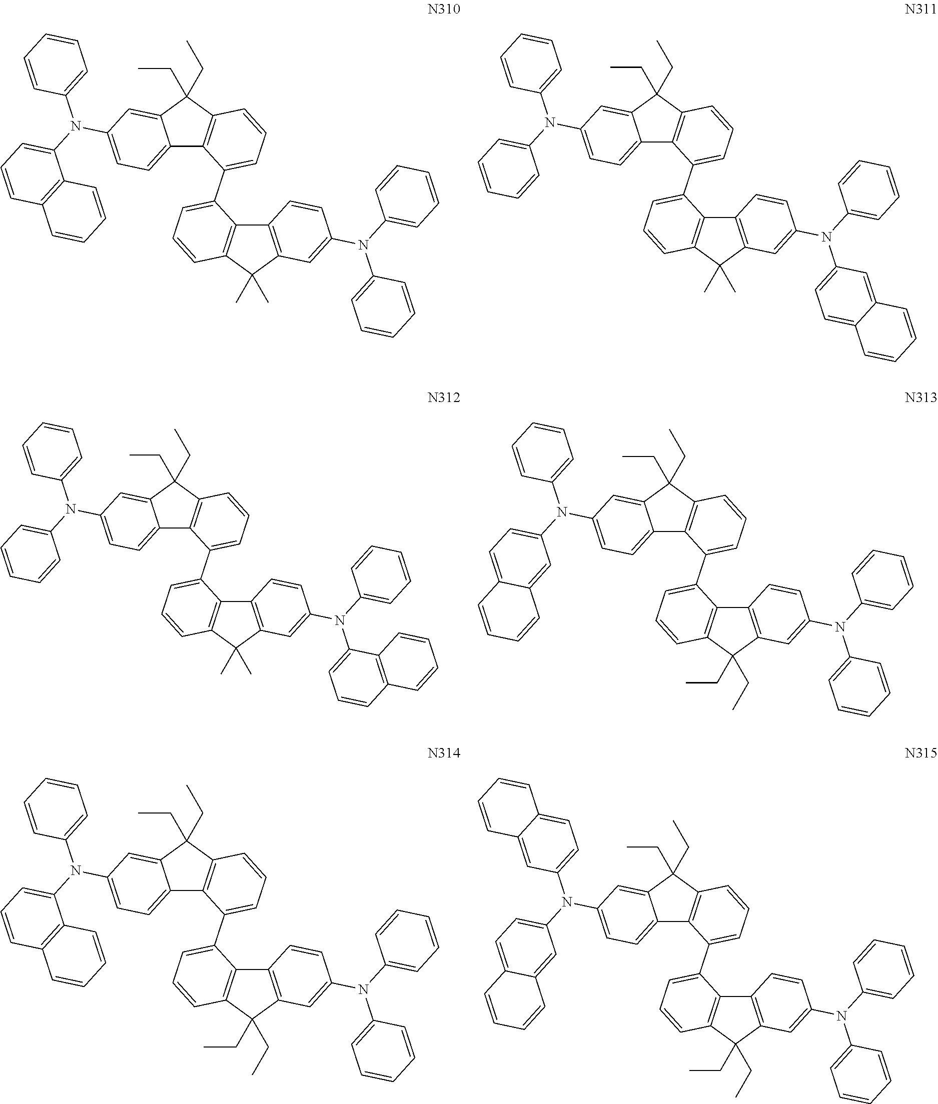

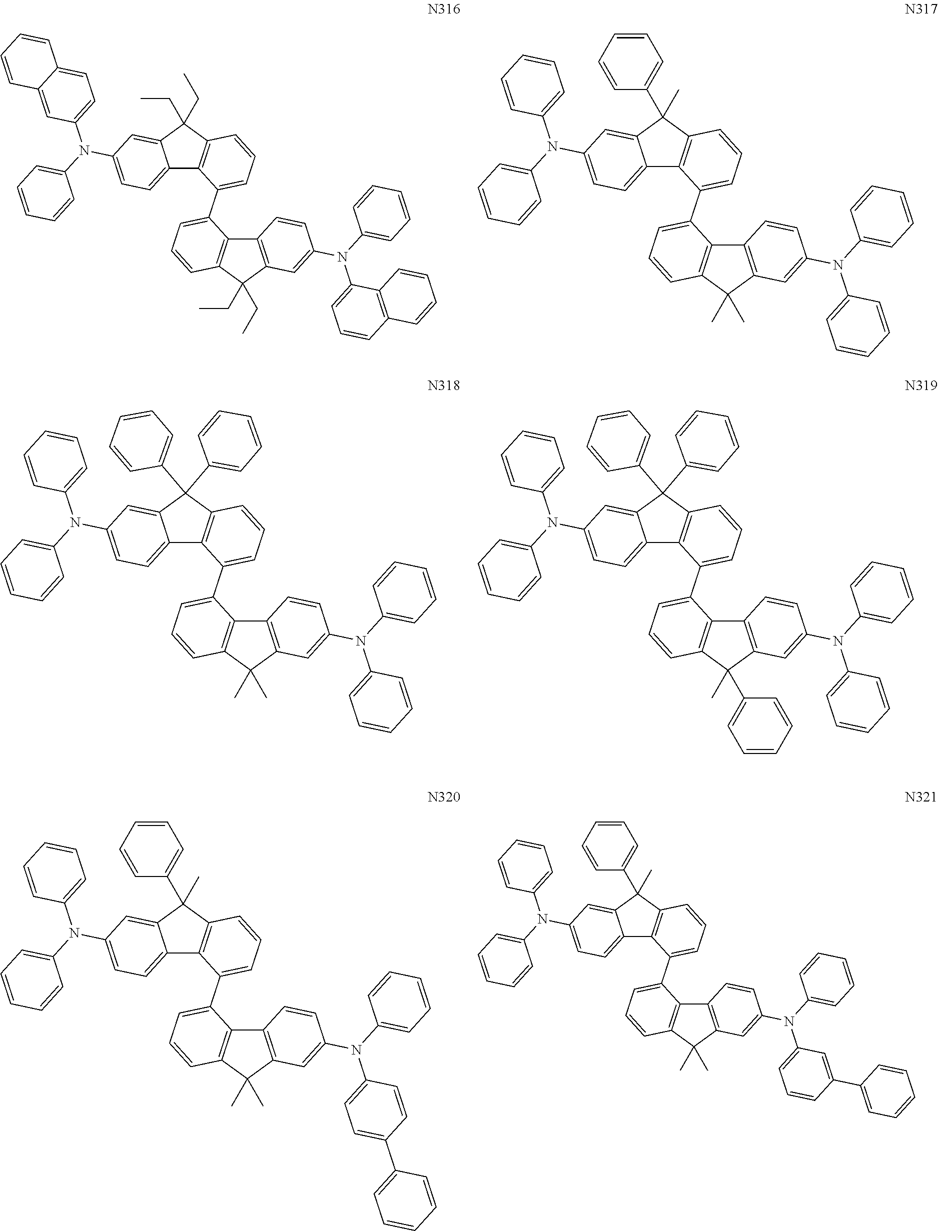

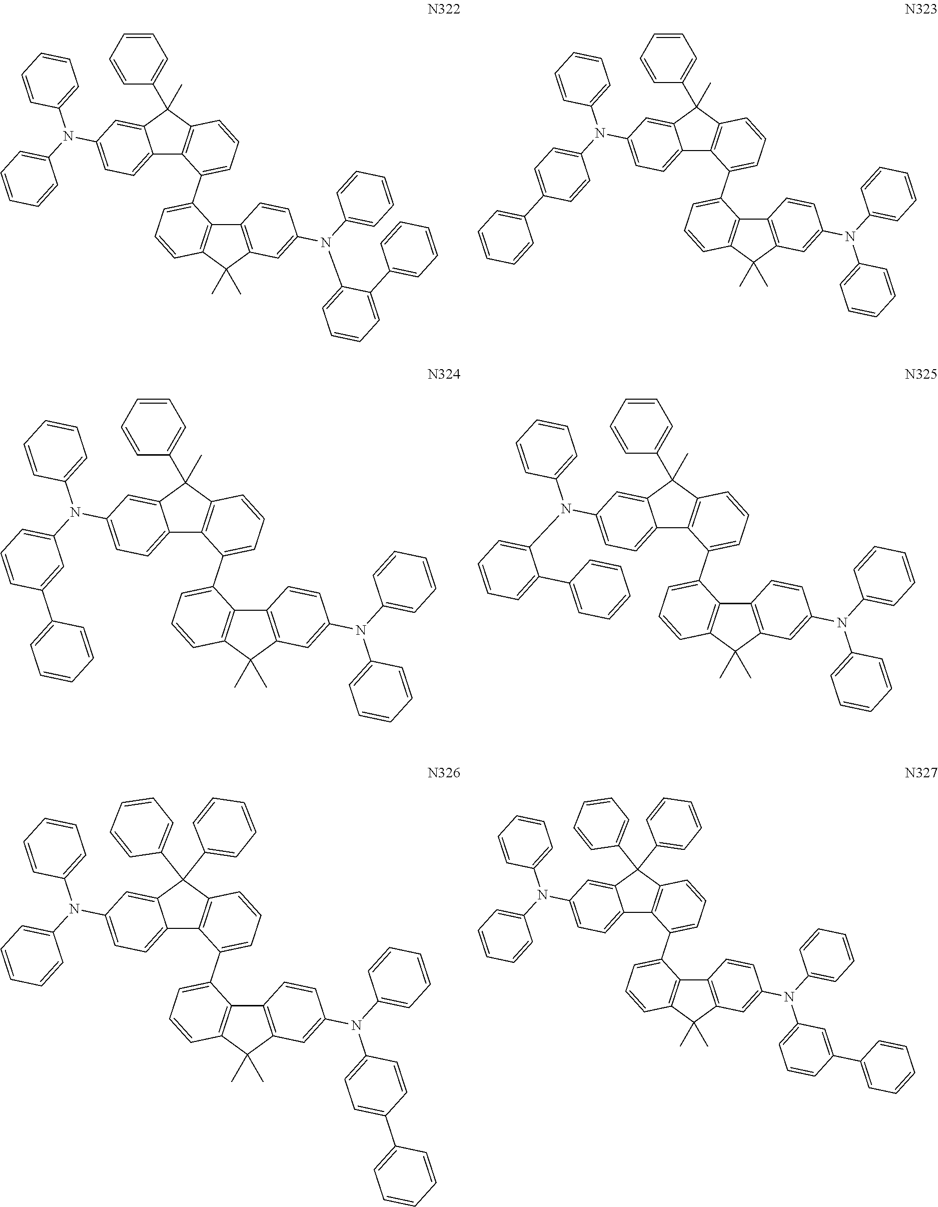

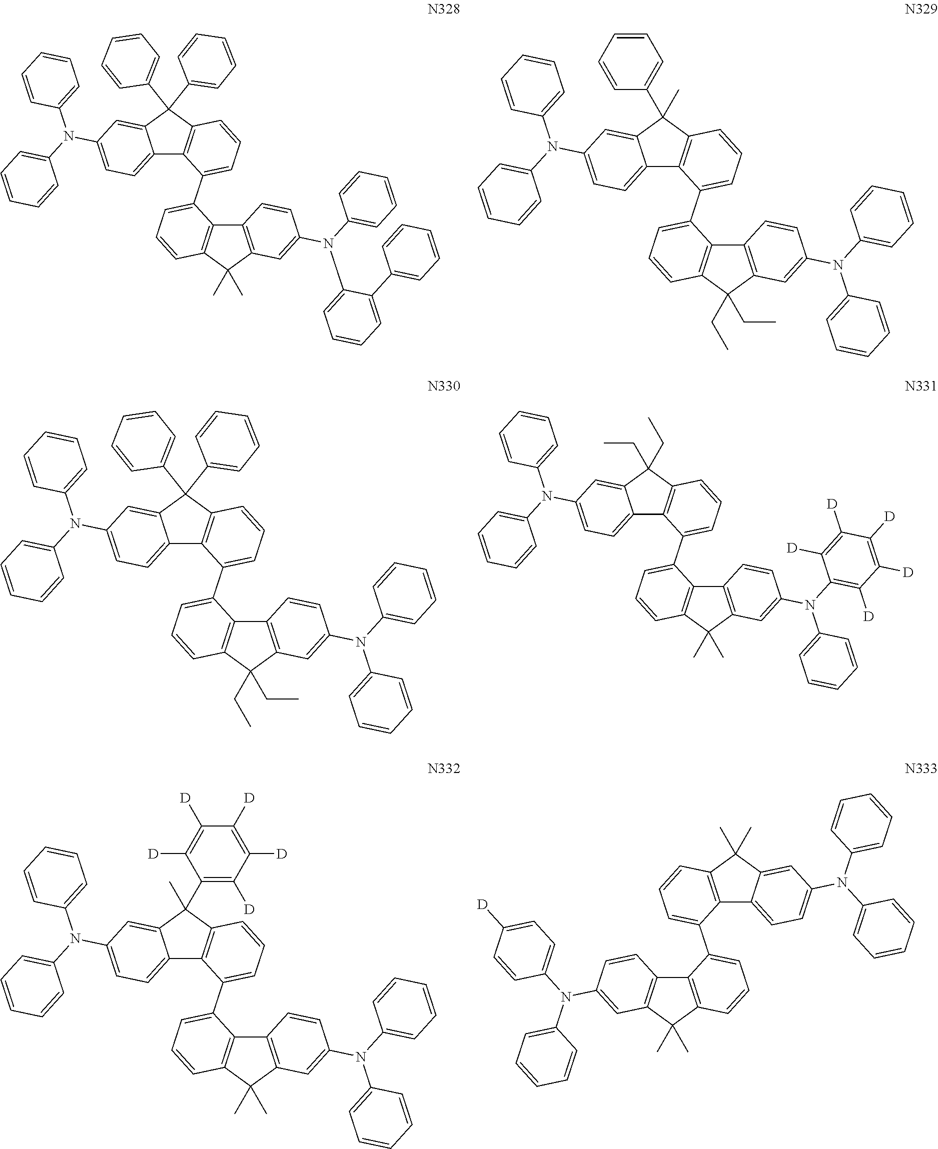

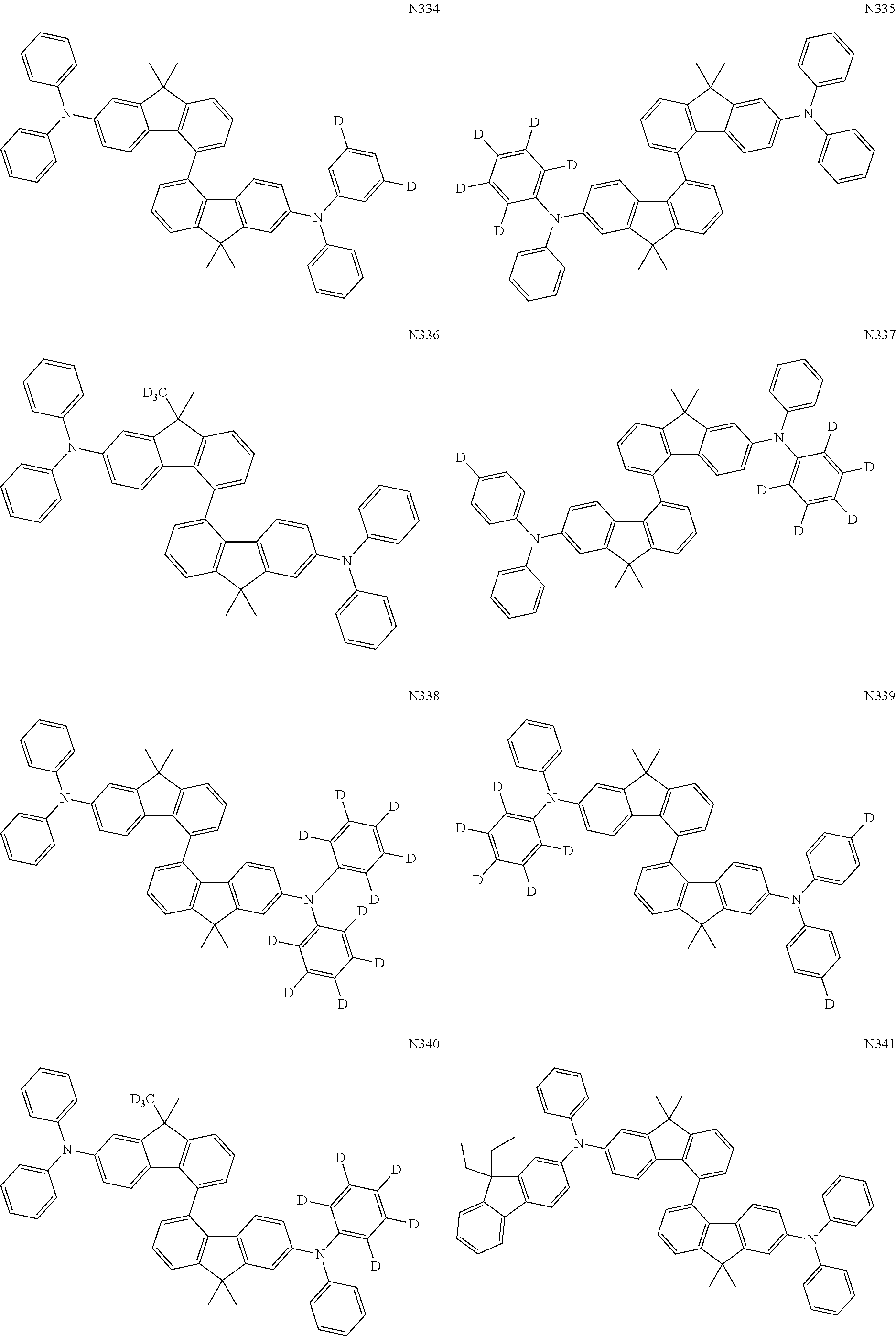

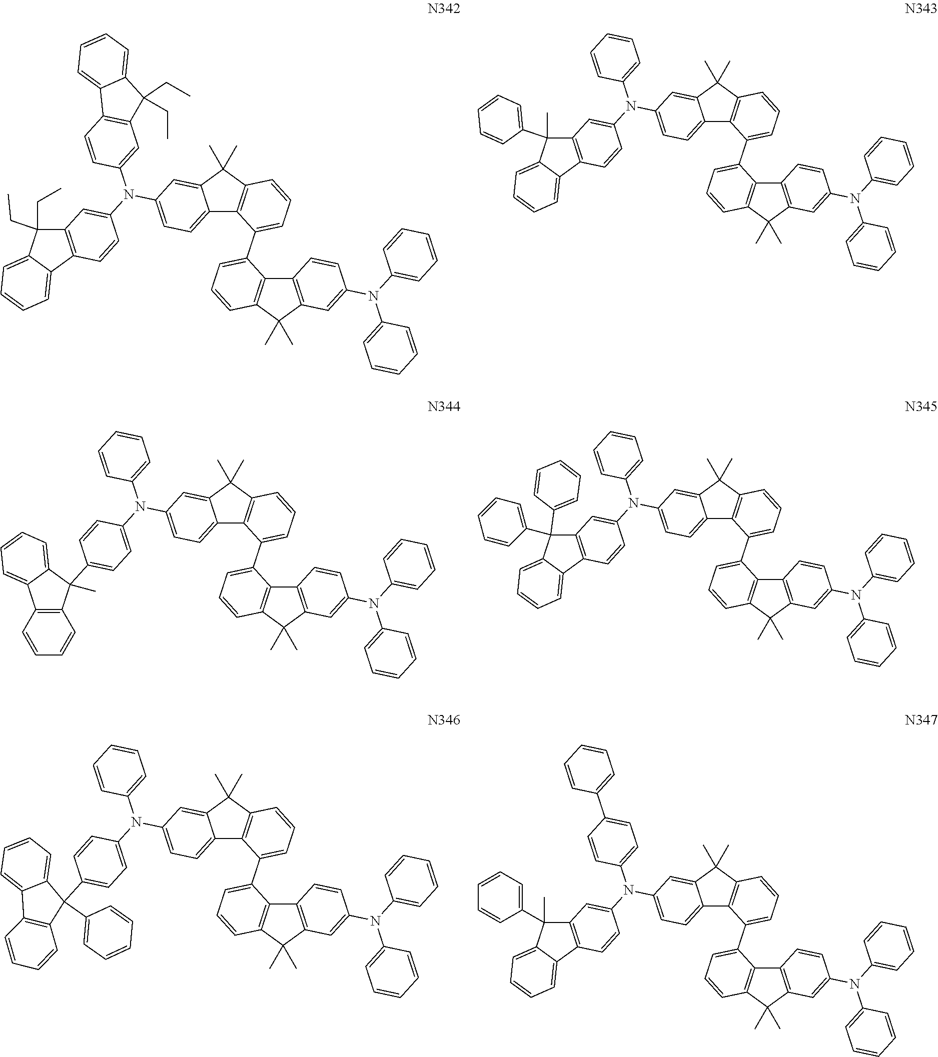

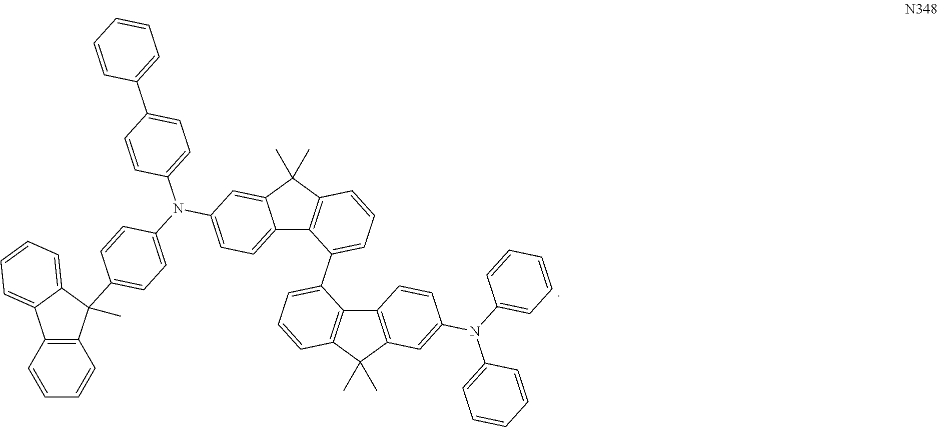 [SMILES: CC1(C)c2cc(N(c3ccccc3)c3ccccc3)ccc2-c2c(-c3cccc4c3-c3ccc(N(c5ccc(-c6ccccc6)cc5)c5ccc(C6(C)c7ccccc7-c7ccccc76)cc5)cc3C4(C)C)cccc21]

11. A light-emitting element comprising:
a pair of electrodes comprising a first electrode and a second electrode;
an organic functional layer disposed between the first electrode and the second electrode;
wherein a material of the organic functional layer comprises an organic compound having a structure as shown in formula (1): 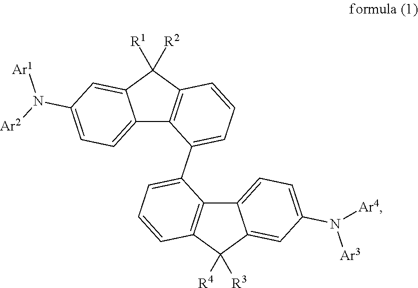
wherein Ar¹, Ar², Ar³ and Ar⁴ are each independently selected from a substituted or unsubstituted aromatic group having 6 to 20 ring atoms and a substituted or unsubstituted heteroaromatic group having 5 to 30 ring atoms; and
R¹, R², R³ and R⁴ are each independently selected from a substituted or unsubstituted methyl, a substituted or unsubstituted ethyl, a substituted or unsubstituted phenyl.

12. The light-emitting element according to claim 11, wherein the organic functional layer comprises a light-emitting layer, a hole transport layer disposed between the light-emitting layer and the first electrode, a light-emitting auxiliary layer disposed between the hole transport layer and the light-emitting layer, a hole injection layer between the hole transport layer and the first electrode, an electron transport layer disposed between the light-emitting layer and the second electrode, and an electron injection layer disposed between the electron transport layer and the second electrode;
wherein the hole transport layer comprises the organic compound.

13. The light-emitting element according to claim 11, wherein Ar¹, Ar², Ar³, Ar⁴ are each independently selected from groups as follows: 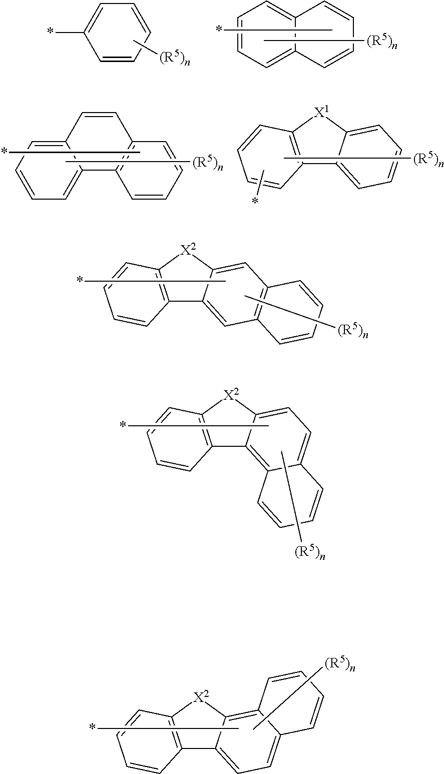
wherein X¹ and X² are each independently selected from O, S, N-Ph, CR⁶R⁷;
R⁵, R⁶ and R⁷ are each independently selected from hydrogen, deuterium, a substituted or unsubstituted linear alkyl group having 1 to 20 carbon atoms, a substituted or unsubstituted branched alkyl group or cyclic alkyl group having 3 to 20 carbon atoms, a substituted or unsubstituted aromatic group having 6 to 10 ring atoms;
N is selected from any integer from 0 to 9; and
* indicates a linkage site.

14. The light-emitting element according to claim 13, wherein X¹ is selected from O, S, N-Ph, CR⁶R⁷, and X² is selected from O, S, CR⁶R⁷;
each occurrence of R⁵ is independently selected from hydrogen, deuterium, a substituted or unsubstituted methyl, a substituted or unsubstituted branched alkyl or cyclic alkyl having 3 to 10 carbon atoms, a substituted or unsubstituted phenyl, a substituted or unsubstituted naphthyl; and
R⁶ and R⁷ are each independently selected from a substituted or unsubstituted methyl, a substituted or unsubstituted ethyl, a substituted or unsubstituted phenyl.

15. The light-emitting element according to claim 14, wherein each occurrence of R⁵ is independently selected from hydrogen, deuterium, an unsubstituted methyl, an unsubstituted isopropyl, an unsubstituted tert-butyl, an unsubstituted cyclohexyl, an unsubstituted adamantyl, an unsubstituted phenyl, an unsubstituted naphthyl; and
R⁶ and R⁷ are each independently selected from an unsubstituted methyl, an unsubstituted ethyl, an unsubstituted phenyl.

16. A display panel comprising a light-emitting element, the light-emitting element comprises:
a pair of electrodes comprising a first electrode and a second electrode;
an organic functional layer disposed between the first electrode and the second electrode;
wherein a material of the organic functional layer comprises an organic compound having a structure as shown in formula (1): 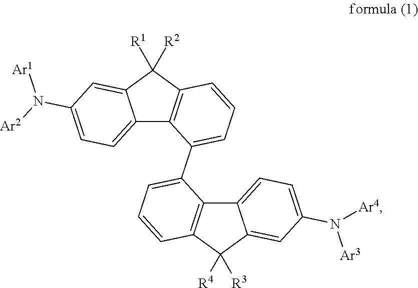
wherein Ar¹, Ar², Ar³ and Ar⁴ are each independently selected from a substituted or unsubstituted aromatic group having 6 to 20 ring atoms and a substituted or unsubstituted heteroaromatic group having 5 to 30 ring atoms; and
R¹, R², R³ and R⁴ are each independently selected from a substituted or unsubstituted methyl, a substituted or unsubstituted ethyl, a substituted or unsubstituted phenyl.

17. The display panel according to claim 16, wherein the organic functional layer comprises a light-emitting layer, a hole transport layer disposed between the light-emitting layer and the first electrode, a light-emitting auxiliary layer disposed between the hole transport layer and the light-emitting layer, a hole injection layer between the hole transport layer and the first electrode, an electron transport layer disposed between the light-emitting layer and the second electrode, and an electron injection layer disposed between the electron transport layer and the second electrode;
wherein the hole transport layer comprises the organic compound.

18. The display panel according to claim 16, wherein Ar¹, Ar², Ar³, Ar⁴ are each independently selected from groups as follows: 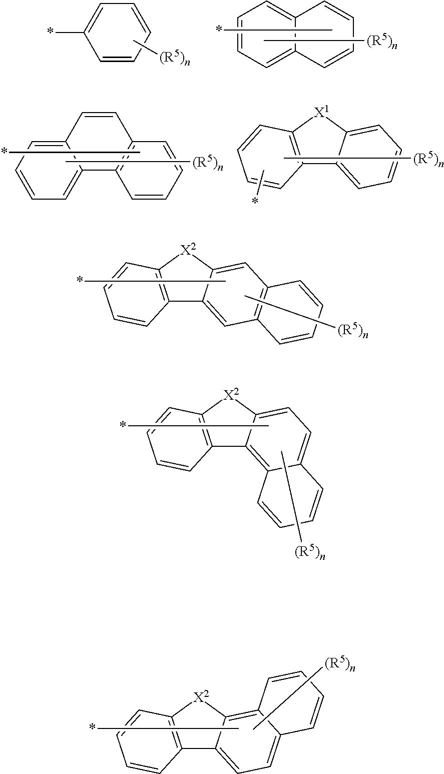
wherein X¹ and X² are each independently selected from O, S, N-Ph, CR⁶R⁷;
R⁵, R⁶ and R⁷ are each independently selected from hydrogen, deuterium, a substituted or unsubstituted linear alkyl group having 1 to 20 carbon atoms, a substituted or unsubstituted branched alkyl group or cyclic alkyl group having 3 to 20 carbon atoms, a substituted or unsubstituted aromatic group having 6 to 10 ring atoms;
N is selected from any integer from 0 to 9; and
* indicates a linkage site.

19. The display panel according to claim 18, wherein X¹ is selected from O, S, N-Ph, CR⁶R⁷, and X² is selected from O, S, CR⁶R⁷;
each occurrence of R⁵ is independently selected from hydrogen, deuterium, a substituted or unsubstituted methyl, a substituted or unsubstituted branched alkyl or cyclic alkyl having 3 to 10 carbon atoms, a substituted or unsubstituted phenyl, a substituted or unsubstituted naphthyl; and
R⁶ and R⁷ are each independently selected from a substituted or unsubstituted methyl, a substituted or unsubstituted ethyl, a substituted or unsubstituted phenyl.

20. The display panel of claim 19, wherein each occurrence of R⁵ is independently selected from hydrogen, deuterium, an unsubstituted methyl, an unsubstituted isopropyl, an unsubstituted tert-butyl, an unsubstituted cyclohexyl, an unsubstituted adamantyl, an unsubstituted phenyl, an unsubstituted naphthyl; and
R⁶ and R⁷ are each independently selected from an unsubstituted methyl, an unsubstituted ethyl, an unsubstituted phenyl.
